(19) United States
(12) Patent Application Publication
(10) Pub. No.: US 2025/0136586 A1
(43) Pub. Date: May 1, 2025

(54) Substituted 7-(Pyrimidin-4-yl)Quinolin-4(1H)-One Compounds as Cyclin Dependent Kinase Inhibitors

(71) Applicant: BeiGene, Ltd., Camana Bay (KY)

(72) Inventors: Jing LI, Beijing (CN); Wenqing Xu, Beijing (CN); Zhiwei Wang, Beijing (CN)

(73) Assignee: BeiGene, Ltd., Camana Bay (KY)

(21) Appl. No.: 18/926,544

(22) Filed: Oct. 25, 2024

Related U.S. Application Data
(63) Continuation of application No. PCT/CN23/91482, filed on Apr. 28, 2023.

(30) Foreign Application Priority Data
Apr. 29, 2022 (WO) PCT/CN22/90343

(51) Int. Cl. C07D 405/14; A61K 31/506; A61K 31/5377; A61K 31/541; A61K 31/55; A61K 31/553; C07D 471/10; C07D 491/048; C07D 491/08; C07D 491/107

(52) U.S. Cl. CPC C07D 405/14; A61K 31/506; A61K 31/5377; A61K 31/541; A61K 31/55; A61K 31/553; C07D 471/10; C07D 491/048; C07D 491/08; C07D 491/107

(57) ABSTRACT

This disclosure provides compounds containing 7-(pyrimidin-4-yl)quinolin-4(1H)-one structure, the use thereof for selectively inhibiting the activity of CDK4, and pharmaceutical compositions comprising the compounds as treatment of various diseases including cancer.

### CROSS-REFERENCE TO RELATED APPLICATIONS

This application is a continuation of International Application No. PCT/CN2023/091482, filed Apr. 28, 2023, which claims priority to International Application No. PCT/CN2022/090343, filed Apr. 29, 2022. The disclosures of each of the aforementioned applications are incorporated herein by reference in their entireties.

### FIELD OF THE INVENTION

This disclosure provides compounds containing 7-(pyrimidin-4-yl)quinolin-4(1H)-one structure, the use thereof for selectively inhibiting the activity of cyclin-dependent kinase 4 (CDK4), and pharmaceutical compositions comprising the compounds as the treatment of various diseases including cancer.

### BACKGROUND OF THE INVENTION

Human kinase is a large group of enzymes that add phosphate groups (PO₄³⁻) to other molecules in human body [1. FASEB J. 1995 May; 9(8):576-96. 2. Enzyme Res. 2011; 2011: 794089.]. There are more than 500 kinase-encoding genes that exist in the human genome and their substrates including proteins, lipids, and nucleic acids [3. Cell Signal. 2004 September; 16(9):983-9. 4. Cell. 2017 Aug. 10; 170(4):605-635.]. Kinase mis-regulation is identified in many diseases including cancer, autoimmunity, neurological disorders, diabetes and cardiovascular disease. For example, the mutated kinases can become constitutively active and thus cause diverse cellular anomalies, leading to cancer initiation or growth. Using small molecular inhibitors to inhibit kinase activity is proved to be a successful method to treat cancer and other disease [5. Expert Rev Anticancer Ther. 2018 December; 18(12):1249-1270.]. Up to now, there are more than 70 kinase inhibitors have been approved by FDA, EMA or CDE as drugs [6. Nat Rev Drug Discov. 2018 May; 17(5):353-377.].

Protein kinase family takes a majority fraction of the kinase superfamily. For protein targets, protein kinases can phosphorylate the amino acids including serine, threonine, tyrosine and histidine. [7. Science. 2002 Dec. 6; 298(5600):1912-34.] Protein kinases play a major role in cellular activation processes, through reversible phosphorylation and dephosphorylation of proteins, by the antagonistic action of kinases and phosphatases, is an important component of cell signaling because the phosphorylated and unphosphorylated states of the target protein can have different levels of activity. [8. Biochimie. 2014 December; 107 Pt B: 167-87. 9. Clin Transl Oncol. 2006 March; 8(3):153-60.] Different protein kinases including EGFR, BTK, ALK, JAK, PI3K and CDK are proved to be good targets for cancer drug development.

Excessively activated cell cycle is a common feature of human cancer [10. Nat Rev Cancer. 2009 March; 9(3):153-66.]. While cyclins are among the most important core cell cycle regulators. There are four basic cyclin types found in humans including G1 cyclins, G1/S cyclins, S cyclins and M cyclins. To drive the cell cycle forward, a cyclin must activate or inactivate many target proteins inside of the cell. And these cyclins drive the events of the cell cycle majorly by partnering with a family of enzymes called the cyclin-dependent kinases (CDKs). CDK itself is inactive, but binding with a cyclin can activate it, making the CDK/cyclin complex a functional holoenzyme and allowing it to modify target proteins [11. Orphanet J Rare Dis. 2020 Aug. 6; 15(1):203. 12. J Mol Biol. 1999 Apr. 16; 287(5):821-8.]. There are 26 serine/threonine protein kinases that form a CDK and CDK-like branch of the CMGC subfamily of the human kinome; of these, 21 are classified as CDKs. Among all the currently identified CDKs, CDK1, CDK2, CDK4 and CDK6 are considered as the direct modulator of cell cycle majorly by phosphorylating and inactivating retinoblastoma protein and releasing E2F transcription factors, and E2F downstream pathway is critical in regulating the initiation of DNA replication. And CDK4/6 is essential for G1 early initiation and G1/S transition. [13. Cell Death Differ. 1998 February; 5(2):132-40. 14. Oncogene. 2016 Sep. 15; 35(37):4829-35.]

CDK4/6 related pathway is commonly disregulated in many different cancer types such as breast cancer, lung cancer and pancreatic cancer. And there are 4 approved CDK4/6 inhibitors including palbociclib, ribociclib, abemaciclib and trilaciclib which have been approved by FDA or CDE to be used as either single agent or combo with endocrine therapy to treat HR+, Her2− breast cancer. This approach shows good efficacy in the clinic while hematopoietic toxicity like neutropenia and leukopenia are also observed, which may limit the clinical application of CDK4/6 dual inhibitors. And emerging data indicates that inhibition of CDK6/Cyclin D3 may cause the clinical observed hematologic toxicity [15. Cell. 2004 Aug. 20; 118(4):493-504. 16. Haematologica. 2021 Oct. 1:106(10):2624-2632.] while CDK4/Cyclin D1 is the oncogenic driver in different cancers [17. Nat Commun. 2019 Dec. 20; 10(1):5817. 18. 18. Cancer Cell. 2006 January; 9(1):23-32.]. Development of a selective CDK4 inhibitor might show clinical benefits including improved efficacy, mitigated hematologic toxicity and expanded usage in many cancers including but not limited to breast cancer, lung cancer, pancreatic cancer, prostate cancer, bone cancer, liver cancer and endometrial cancer.

Thus there remains a great need to develop a selective CDK4 inhibitor. Here, the inventors of the instant invention found the selective CDK4 inhibitor compounds which potentially lead to better efficacy, improved toxicity profile and the potential to overcome resistance mechanisms, and the like.

### SUMMARY OF THE INVENTION

One objective of the present invention is to provide compounds and derivatives which function to act as CDK4 inhibitors, and methods of preparation and uses thereof.

Aspect 1. A compound of formula (I):

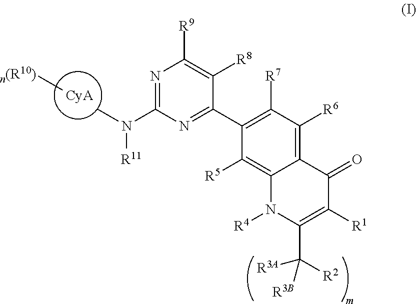

or a N-oxide thereof, or a pharmaceutically acceptable salt thereof, or a stereoisomer thereof, or a tautomer thereof, or a deuterated analog thereof, or a prodrug thereof,
wherein:
ring CyA is a 3- to 8-membered ring, said ring comprising 0, 1 or 2 heteroatom(s) independently selected from nitrogen, oxygen, or optionally oxidized sulfur as ring member(s); said ring is optionally substituted with at least one substituent R¹⁰;
n is 0, 1, 2, 3, 4 or 5;
m is 0 or 1; provided that when m=0, the

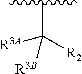

moiety as a whole is replaced with H,
R¹ is H, halogen, —C₁₋₈alkyl, —C₂₋₈alkenyl, —C₂₋₈alkynyl, cycloalkyl, haloalkyl, heterocyclyl, aryl, heteroaryl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1d} and R^{1f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2d};
R^{2a}, R^{2b} and R^{2c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(two R^{2d}) and/or (two R^{2f}) together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent selected from the group consisting of halogen, hydroxy, oxo, —C₁₋₈alkyl, -haloC₁₋₈alkyl, —C(O)C₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2g}, R^{2h} and R²ⁱ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, —C(O)C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteraryl or haloheteroaryl;
R^{3A} and R^{3B} are each independently hydrogen, halogen, —C₁₋₈alkyl, —C₂₋₈alkenyl, —C₂₋₈alkynyl, cycloalkyl, heterocyclyl, aryl, heteroaryl or —CN; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3c}; or
R^{3A} and R^{3B} together with the atom to which they are attached, form an oxo group (—C(═O)—) or a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3d}, R^{3e} and R^{3f} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁴ is hydrogen, —C₁₋₈alkyl, —C₂₋₈alkenyl, —C₂₋₈alkynyl, cycloalkyl or heterocyclyl; wherein each of said —C₁₋₈alkyl, —C₂₋₈alkenyl, —C₂₋₈alkynyl, cycloalkyl or heterocyclyl is optionally substituted with at least one substituent R⁴;
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵, R⁶, R⁷, R⁸ and R⁹ are each independently selected from H, halogen. —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5f};
R^{5d} and R^{5f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹⁰ is selected from H, halogen. —C₁₋₈alkyl, —C₂₋₈alkenyl, —C₂₋₈alkynyl, cycloalkyl, heterocyclyl, aryl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10d};
R^{10a}, R^{10b} and R^{10c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent
R^{10d} and R^{10f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹¹ is selected from H, —C₁₋₈alkyl, —C₂₋₈alkenyl, —C₂₋₈alkynyl; wherein each of said —C₁₋₈alkyl, —C₂₋₈alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

Aspect 2. The compound of Aspect 1, wherein the compound is selected from formula (IIa), (IIb), (IIc), (IId) or (IIe):

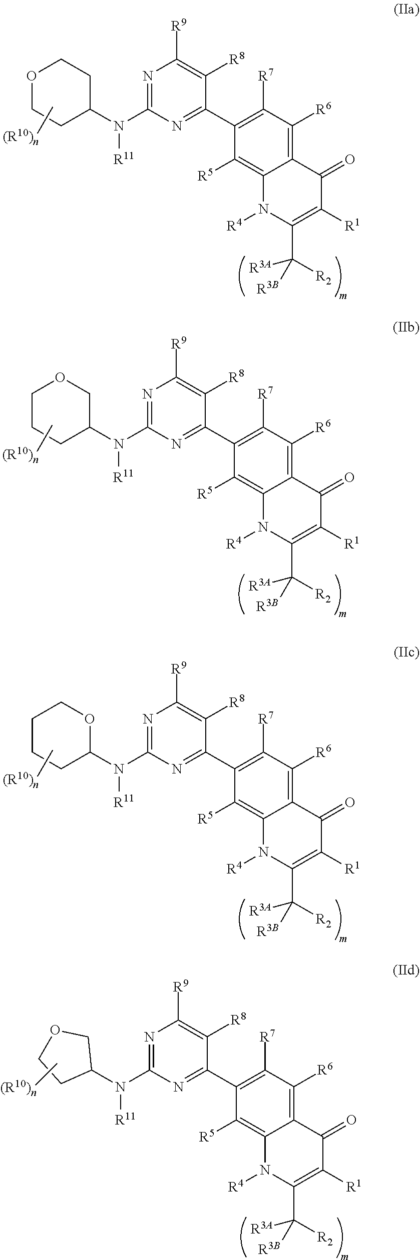

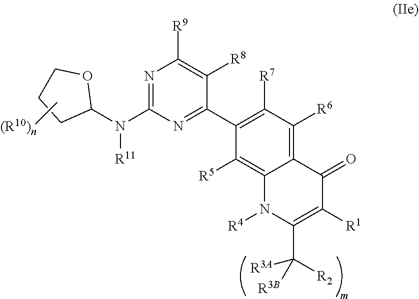

wherein, R¹, R², R^{3A}, R^{3B}, R⁴, R⁵, R⁶, R⁷, R⁸, R⁹, R¹⁰, R¹¹, m and n are each defined as Aspect 1;
preferably, the compound is selected from formula (IIf), (IIg), (IIh) or (IIi):

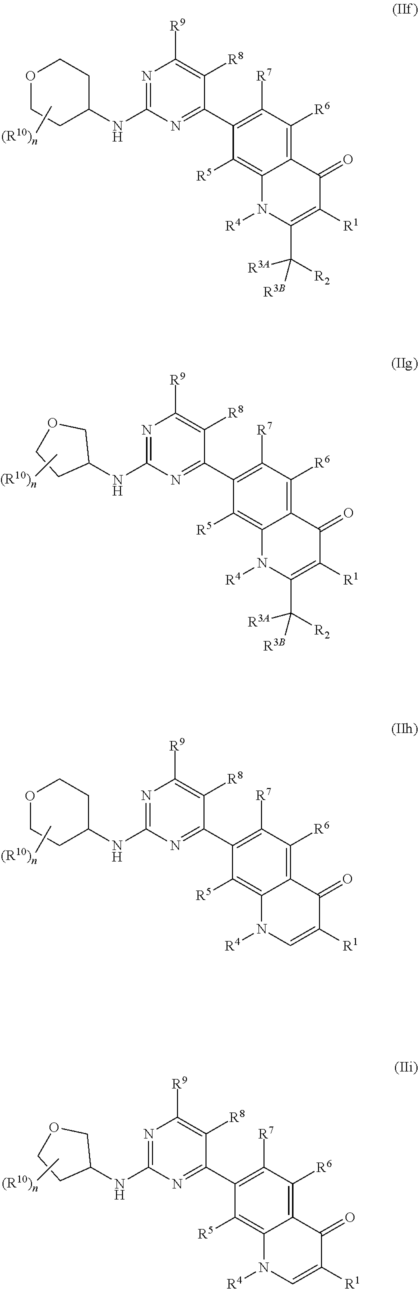

wherein, R¹, R², R^{3A}, R^{3B}, R⁴, R⁵, R⁶, R⁷, R⁸, R⁹, R¹⁰ and n are each defined as Aspect 1;
more preferably, the compound is selected from formula (IIj), (IIk), (IIl) or (IIm):

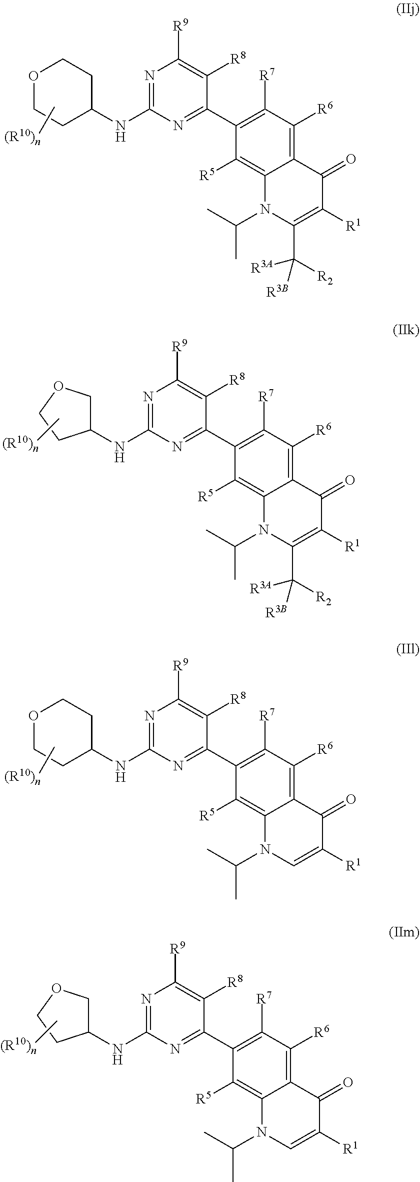

wherein, R¹, R², R^{3A}, R^{3B}, R⁵, R⁶, R⁷, R⁸, R⁹, R¹⁰ and n are each defined as Aspect 1;
even more preferably, the compound is selected from formula (IIn), (IIo), (IIp) or (IIq):

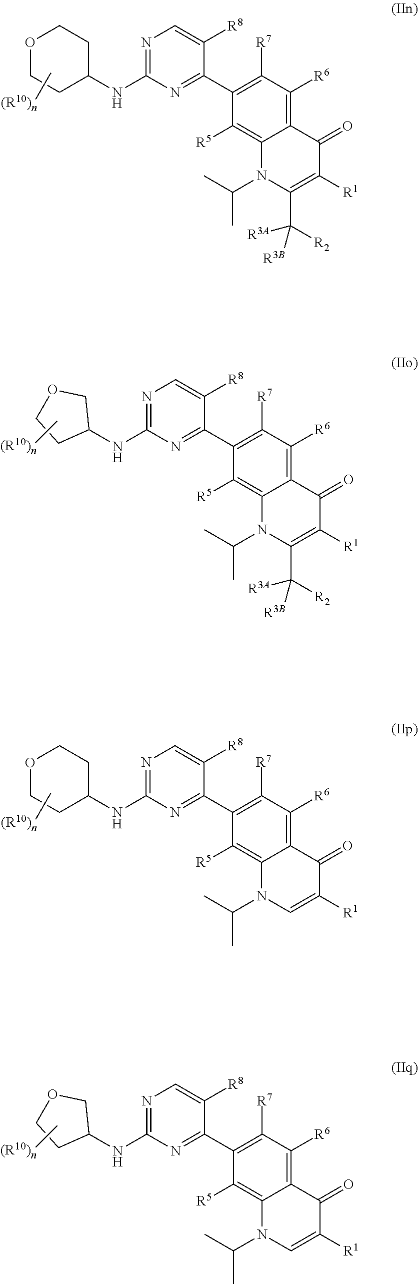

wherein, R¹, R², R^{3A}, R^{3B}, R⁵, R⁶, R⁷, R⁸, R¹⁰, m and n are each defined as Aspect 1.

Aspect 3. The compound of anyone of the preceding Aspects, wherein ring CyA is a 3-, 4-, 5-, 6-, 7- or 8-membered ring, said ring comprising 0, 1 or 2 heteroatom(s) independently selected from nitrogen, oxygen, or optionally oxidized sulfur as ring member(s); said ring is optionally substituted with 0, 1, 2, 3, 4 or 5 R¹⁰; said ring is a saturated or unsaturated ring;
preferably CyA is a 3-, 4-, 5-, 6-, 7- or 8-membered saturated ring, said ring comprising 0, 1 or 2 heteroatom(s) independently selected from nitrogen, oxygen, or optionally oxidized sulfur as ring member(s); said ring is optionally substituted with 0, 1, 2 or 3 R¹⁰;
more preferably CyA is a 5-, 6- or 7-membered saturated ring, said ring comprising 1 or 2 heteroatom(s) independently selected from nitrogen or oxygen as ring member(s); said ring is optionally substituted with 0, 1, 2 or 3 R¹⁰;
even more preferably, CyA is a ring selected from tetrahydrofuranyl or tetrahydropyranyl; said ring is optionally substituted with 0, 1, 2 or 3 R¹⁰.

Aspect 4. The compound of anyone of the preceding Aspects, wherein ring CyA is

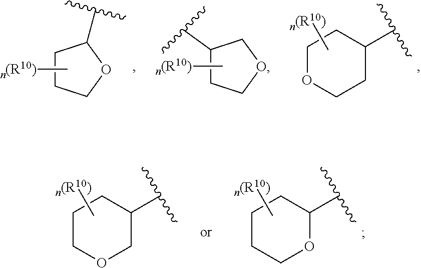

preferably, CyA is

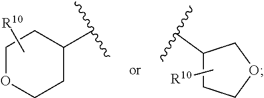

more preferably, CyA is

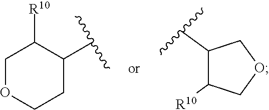

even more preferably, CyA is

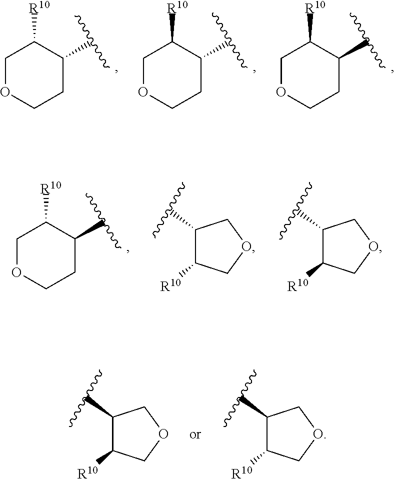

Aspect 5. The compound of anyone of the preceding Aspects, wherein R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10d};
R^{10a}, R^{10b} and R^{10c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10f};
R^{10d} and R^{10f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OH or —NH₂;
more preferably, R¹⁰ is —OH.

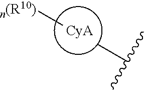

Aspect 6. The compound of anyone of the preceding Aspects, wherein the

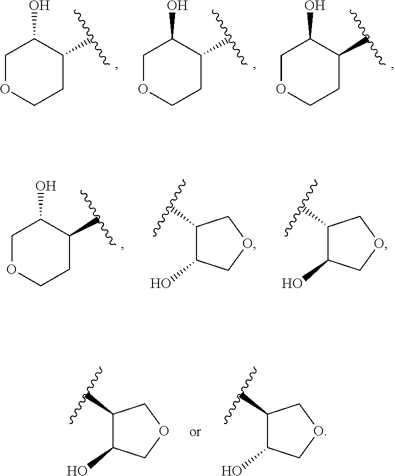

Aspect 7. The compound of anyone of the preceding Aspects, wherein R¹ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl or heterocyclyl is optionally substituted with at least one substituent R^{1d}.
R^{1a}, R^{1b} and R^{1c} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{1f};
R^{1d} and R^{1f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹ is H, —F, —Cl, —Br, —I, methyl, ethyl, propyl, butyl, pentyl, hexyl, heptyl, octyl, —C₂₋₈alkynyl, cyclopropyl, cyclobutyl, cyclopentyl, cyclohexyl, cycloheptyl, cyclooctyl, —OR^{1a}, —COR^{1a};
   wherein each of said methyl, ethyl, propyl, butyl, pentyl, hexyl, heptyl, octyl, —C₂₋₈alkynyl, cyclopropyl, cyclobutyl, cyclopentyl, cyclohexyl, cycloheptyl or cyclooctyl is optionally substituted with at least one substituent selected from hydrogen, —F, —Cl, —Br, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{1a} is selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl;
more preferably, R¹ is H, —F, —Cl, —Br, —I, C₁₋₈alkoxy-C₁₋₈alkyl-, methoxy, ethoxy, propoxy, butoxy, pentoxy, hexoxy, hepthoxy, octoxy, methyl, ethyl, propyl, butyl, pentyl, hexyl, heptyl, octyl, cyclopropyl, cyclobutyl, cyclopentyl, cyclohexyl, cycloheptyl, cyclooctyl, haloalkyl, heterocyclyl or —C(O)C₁₋₈alkyl;
even more preferably, R¹ is H, methyl, ethyl, n-propyl, iso-propyl, n-butyl, sec-butyl, iso-butyl, tert-butyl, —C(O)CH₃, —C(CH₃)₂OH, —OMe, —F, —Cl, cyclopropyl or cyclobutyl.

Aspect 8. The compound of anyone of the preceding Aspects, wherein R² is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2d};
R^{2a}, R^{2b} and R^{2c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) and/or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C(O)—CH₃, —C(O)—CH₂CH₃, —C(O)—CH₂CH₂CH₃, —C(O)—CH₂(CH₃)₂, —C(O)—CH₂CH₂CH₂CH₃, —C(O)—CH(CH₃)CH₂CH₃, —C(O)—CH₂CH(CH₃)CH₃, —C(O)—C(CH₃)₃, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C(O)—CH₃, —C(O)—CH₂CH₃, —C(O)—CH₂CH₂CH₃, —C(O)—CH₂(CH₃)₂, —C(O)—CH₂CH₂CH₂CH₃, —C(O)—CH(CH₃)CH₂CH₃, —C(O)—CH₂CH(CH₃)CH₃, —C(O)—C(CH₃)₃, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 9. The compound of anyone of the preceding Aspects, wherein R² is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2a}, —COR^{2a}, —CO₂R^{2a}, —CONR^{2a}R^{2b}, —NR^{2a}R^{2b} or —NR^{2a}COR^{2b}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d}.
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2g}, —SO₂R^{2g}, —COR^{2g}, —CO₂R^{2g}, —CONR^{2g}R^{2h}, —NO₂, —NR^{2g}R^{2h} or —NR^{2g}COR^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) and/or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C(O)—CH₃, —C(O)—CH₂CH₃, —C(O)—CH₂CH₂CH₃, —C(O)—CH₂(CH₃)₂, —C(O)—CH₂CH₂CH₂CH₃, —C(O)—CH(CH₃)CH₂CH₃, —C(O)—CH₂CH(CH₃)CH₃, —C(O)—C(CH₃)₃, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C(O)—CH₃, —C(O)—CH₂CH₃, —C(O)—CH₂CH₂CH₃, —C(O)—CH₂(CH₃)₂, —C(O)—CH₂CH₂CH₂CH₃, —C(O)—CH(CH₃)CH₂CH₃, —C(O)—CH₂CH(CH₃)CH₃, —C(O)—C(CH₃)₃, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 10. The compound of anyone of the preceding Aspects, wherein R² is hydrogen, methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, —OR^{2a}, —NR^{2a}R^{2b} or —NR^{2a}COR^{2b}; wherein each of said methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl (n-propyl or iso-propyl), butyl (n-butyl, sec-butyl, iso-butyl or tert-butyl), pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, —CF₃, —CF₂H, —CFH₂, —CH₂CF₃, —CF₂CH₃, —CH₂OH, —CH(CH₃)OH, —C(CH₃)₂OH, —CH₂CH₂OH, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OH, methoxy, ethoxy, propoxy, butoxy, pentoxy, hexoxy, hepthoxy, octoxy, —SO₂Me, —SO₂Et, —SO₂C₃H₇, —COMe, —COEt, —COC₃H₇, —NH₂, —NHCH₃, —N(CH₃)₂, —NHC₂H₅, —NHC₃H₇, —NHC₄H₉, —CONH₂, —CONHCH₃, —CON(CH₃)₂, —CONHC₂H₅, —CONHC₃H₇, or —CONHC₄H₉.

Aspect 11. The compound of anyone of the preceding Aspects, wherein R² is —H, -Me, —OMe, —OH, —NH₂, —NHCH₃, —N(CH₃)₂, NHCH(CH₃)₂, —NHC(CH₃)₃, —NHCOCH₃,

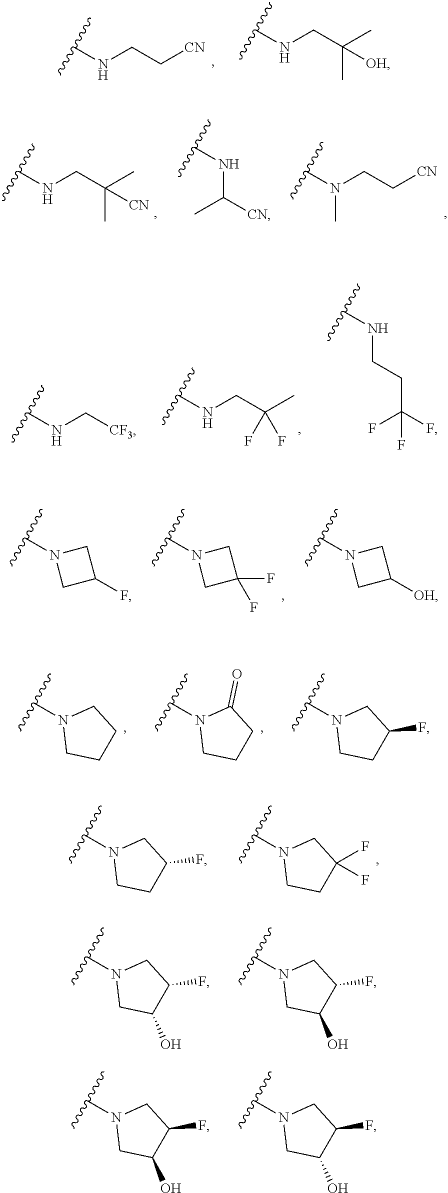

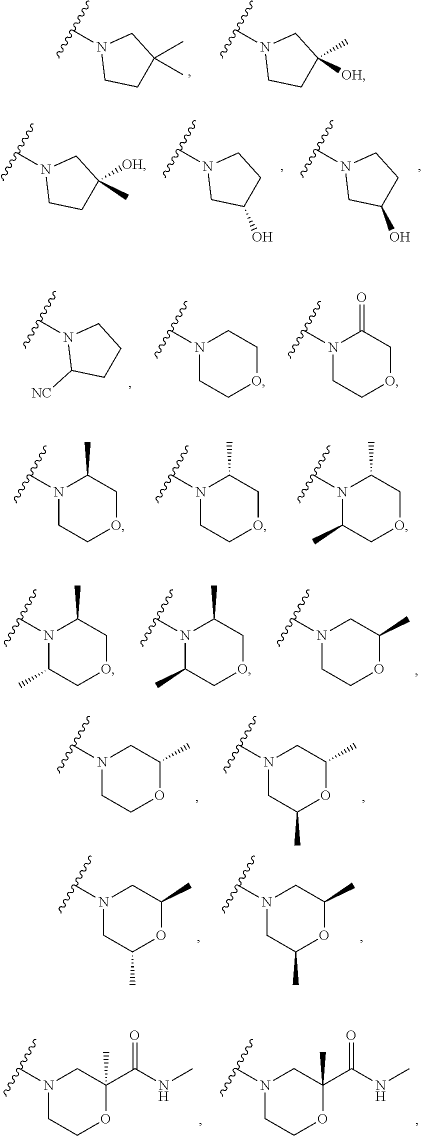

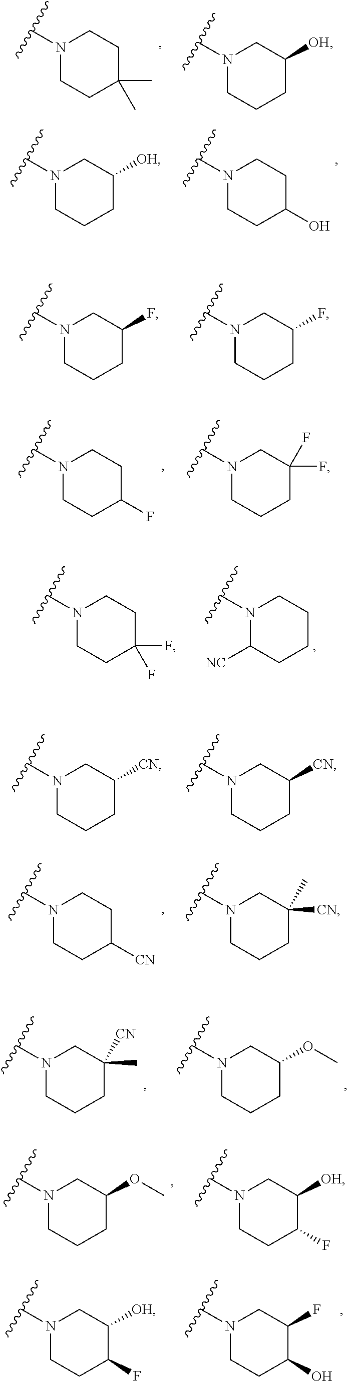

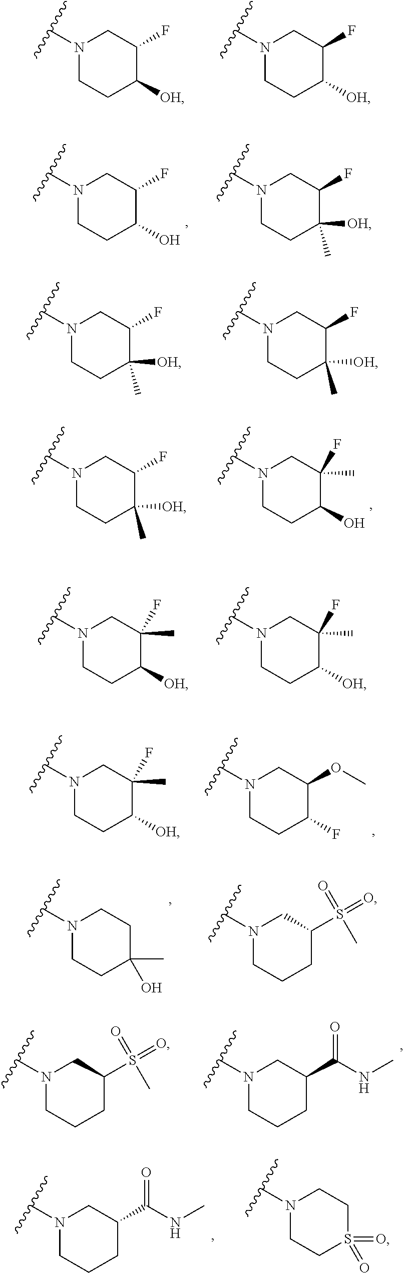

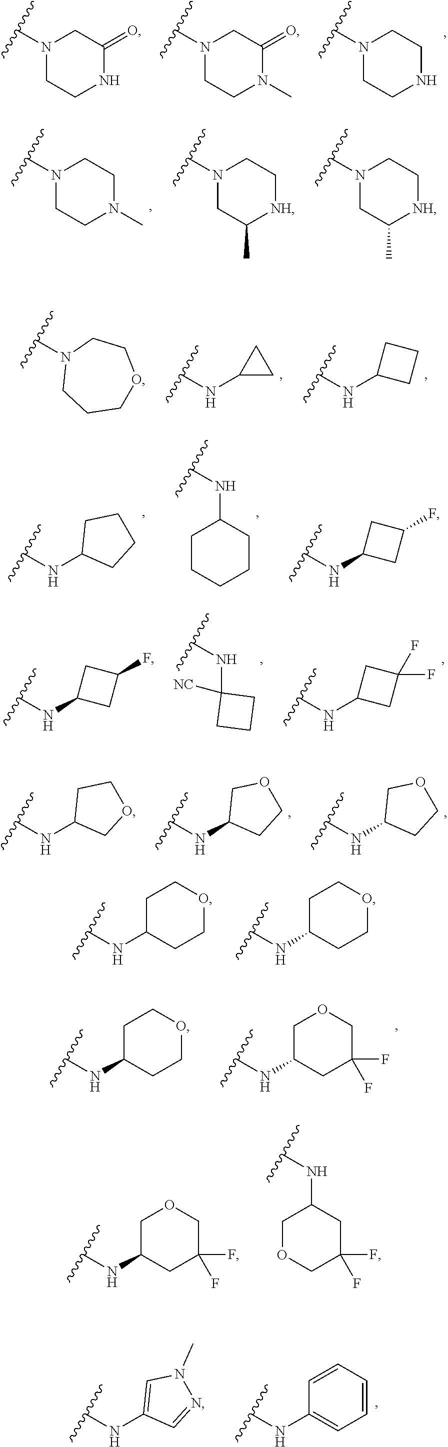

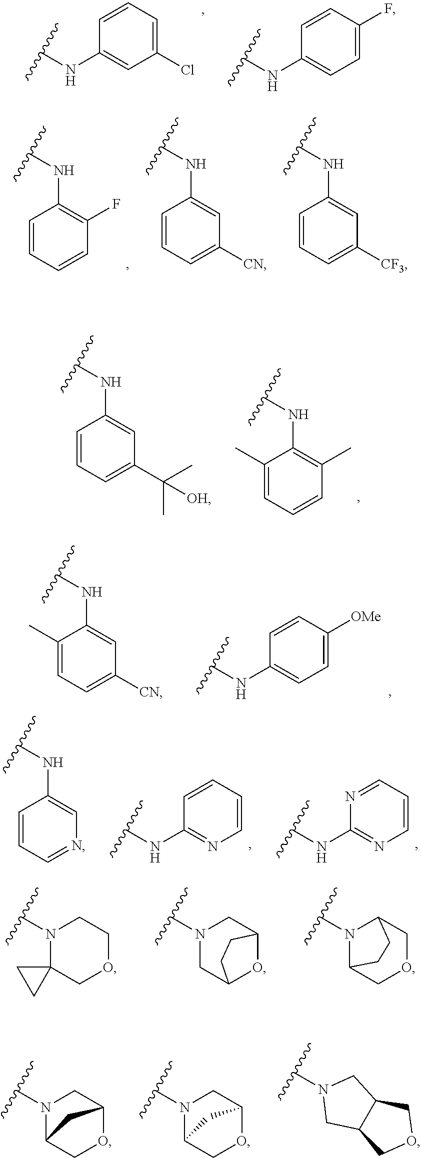

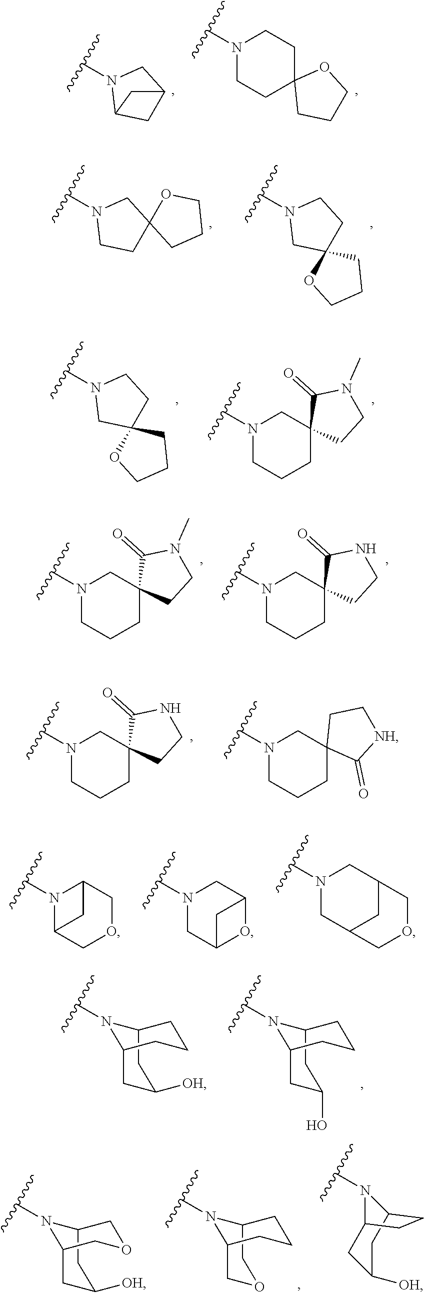

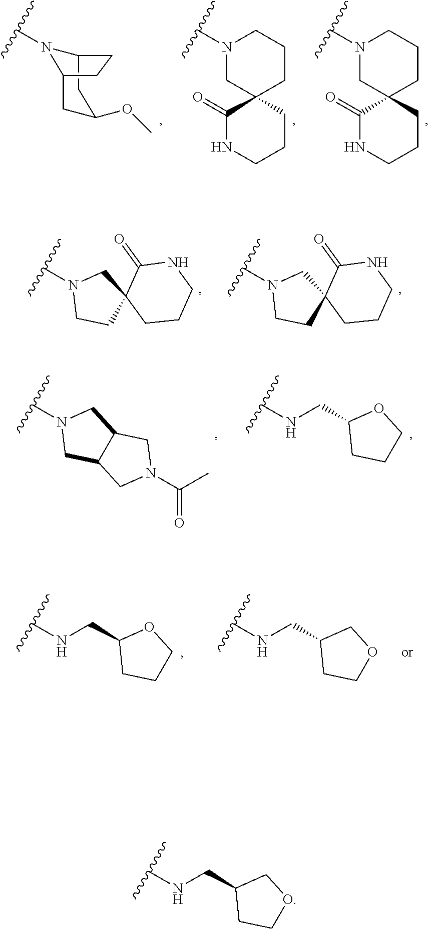

Aspect 12. The compound of anyone of the preceding Aspects, wherein R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3c}; or
R^{3A} and R^{3B} together with the atom to which they are attached, form an oxo group (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d}, R^{3e} and R^{3f} are each independently selected from hydrogen, methyl, ethyl, propyl, butyl, pentyl, hexyl, heptyl, octyl. —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
preferably, R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; or
R^{3A} and R^{3B} together with the atom to which they are attached, form an oxo group (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s); preferably, R^{3A} and R^{3B} together with the atom to which they are attached, form an oxo group (—C(═O)—);
more preferably, R^{3A} and R^{3B} are each independently hydrogen, methyl, ethyl, propyl, butyl or pentyl; or
R^{3A} and R^{3B} together with the atom to which they are attached, form an oxo group (—C(═O)—) or a 3-, 4-, 5-, 6-, 7- or 8-membered ring, said ring comprising 0, 1 or 2 heteroatom(s) independently selected from nitrogen or oxygen.

Aspect 13. The compound of anyone of the preceding Aspects, wherein the

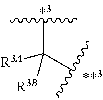

moiety is

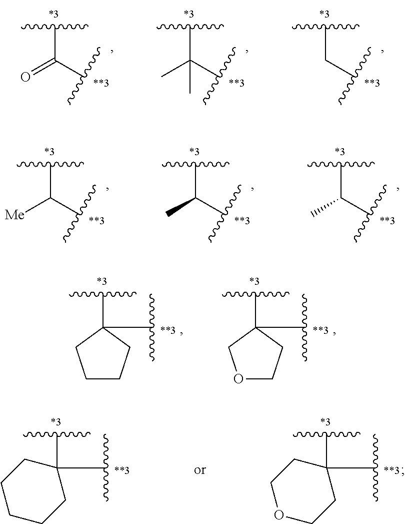

wherein *³ refers to the position attached to

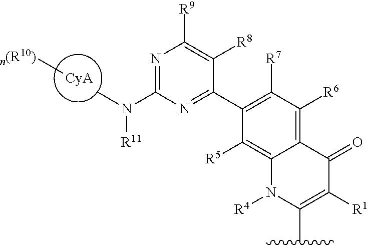

moiety, and **³ refers to the position attached to the

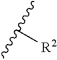

moiety;
in another embodiment, the

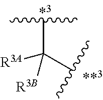

moiety is

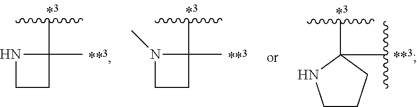

wherein *³ refers to the position attached to

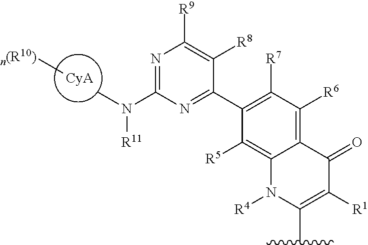

moiety, and **³ refers to the position attached to the

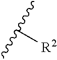

moiety.

Aspect 14. The compound of anyone of the preceding Aspects, wherein the

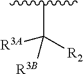

moiety is -Me, -Et,

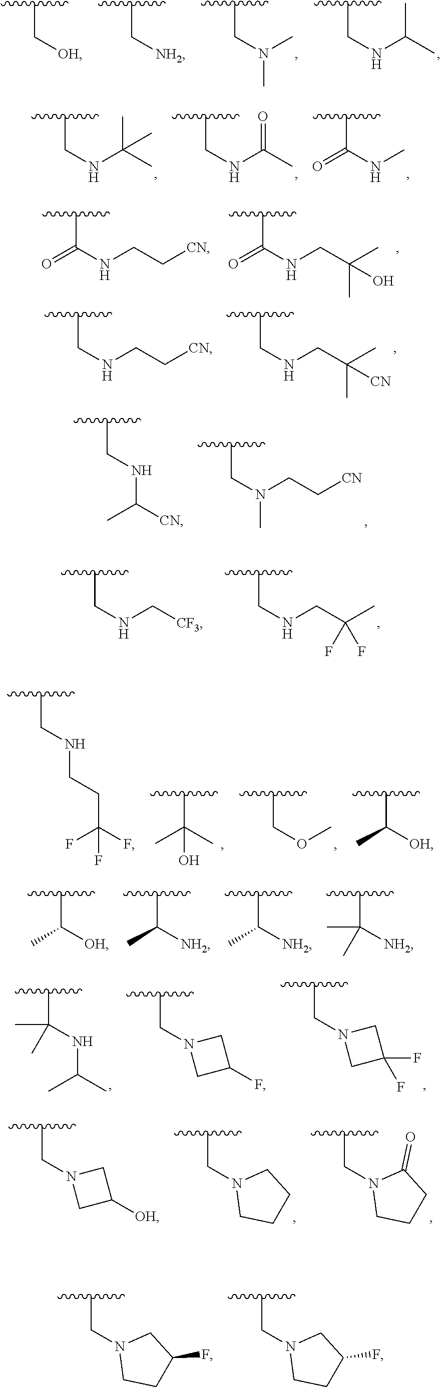

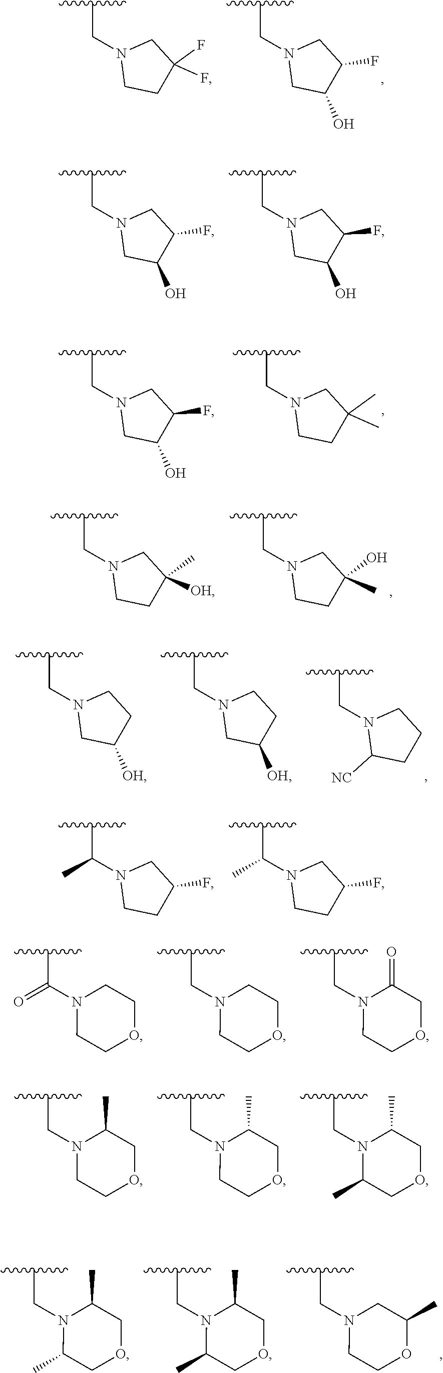

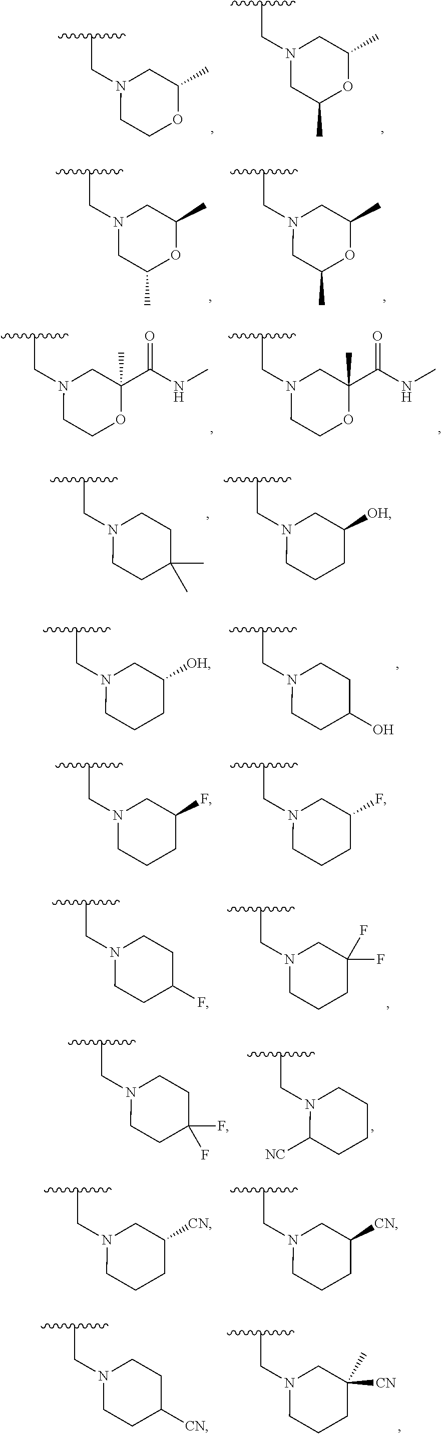

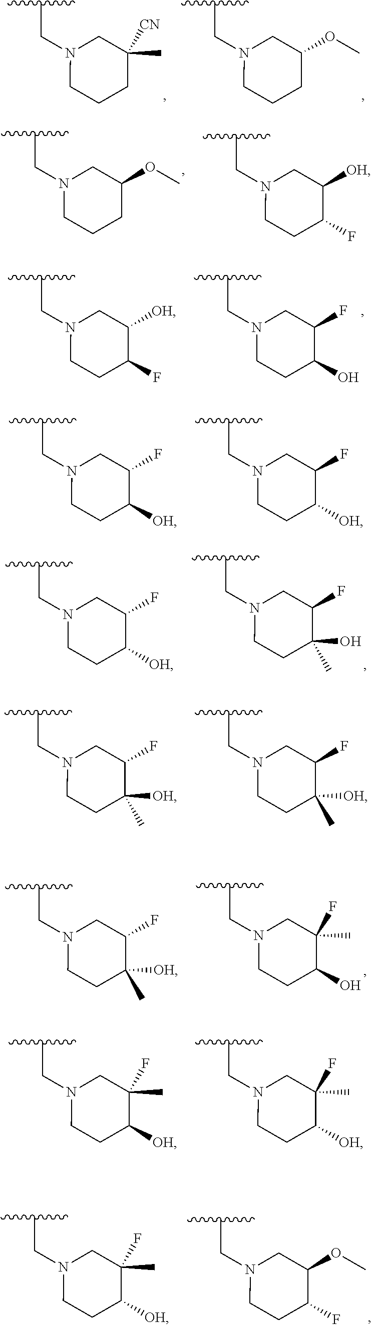

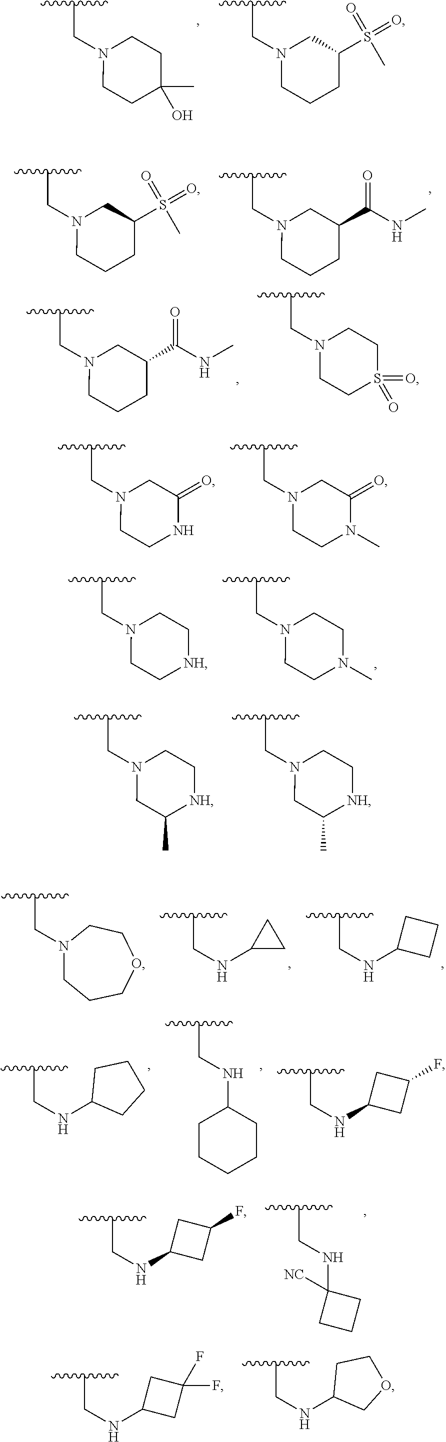

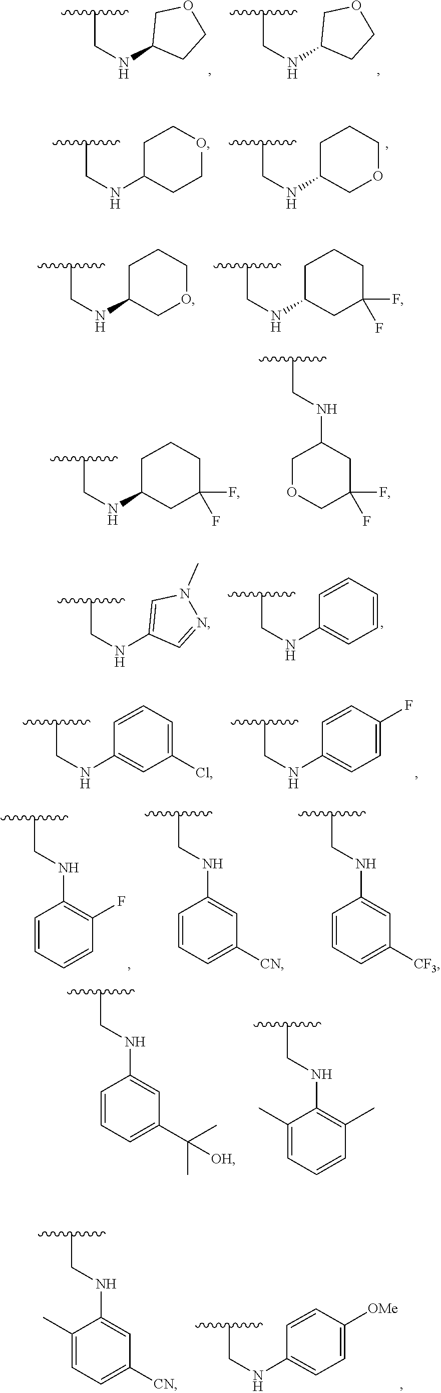

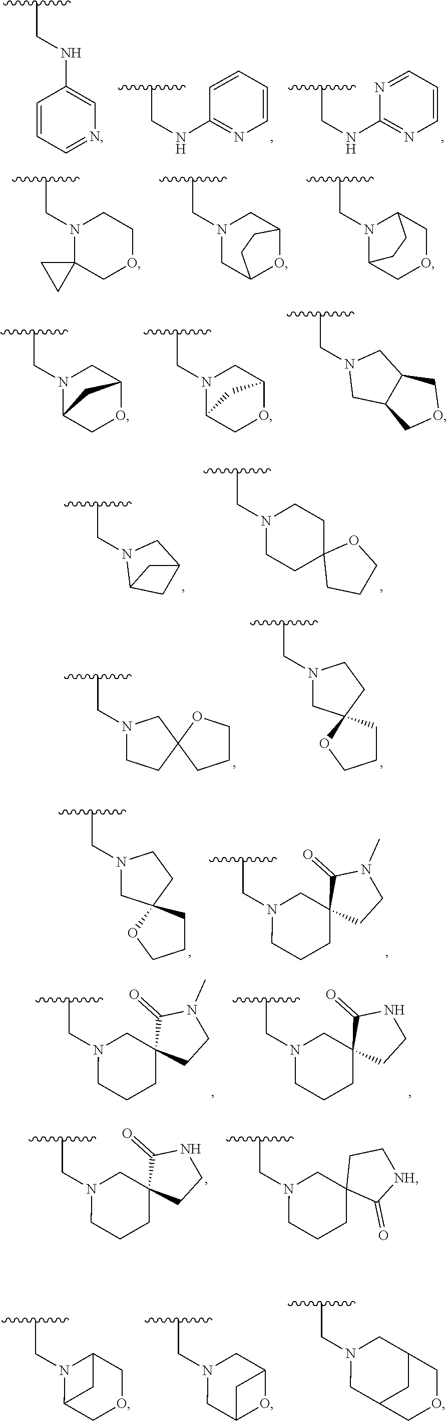

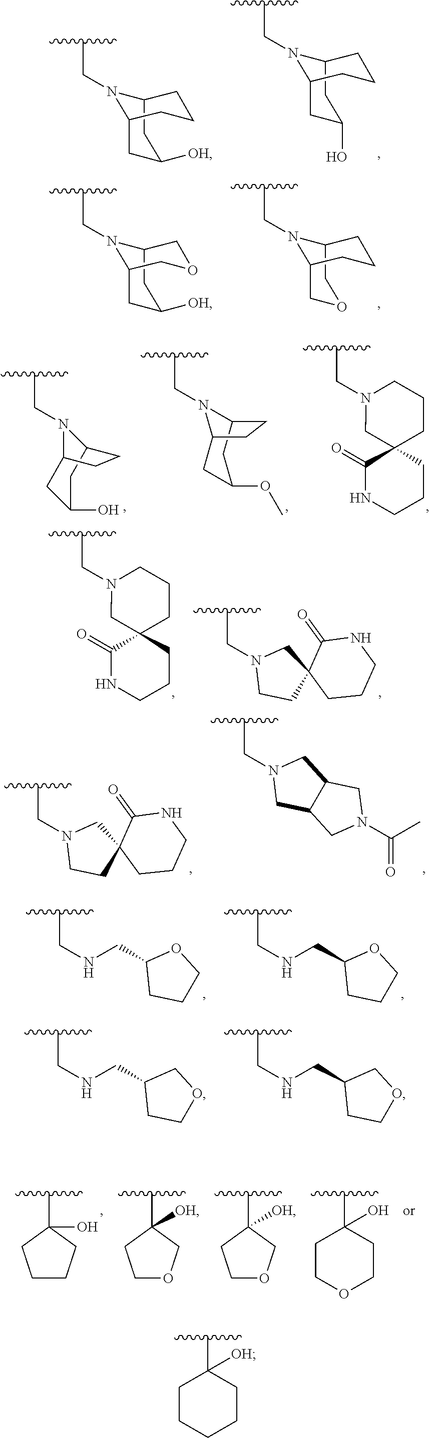

in another embodiment, the

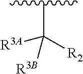

moiety is

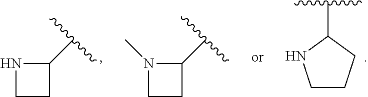

Aspect 15. The compound of anyone of the preceding Aspects, wherein R⁴ is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl is optionally substituted with at least one substituent R^{4a};
R^{4a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁴ is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl;
more preferably, R⁴ is methyl, ethyl, n-propyl, iso-propyl, n-butyl, sec-butyl, iso-butyl, tert-butyl, cyclopropyl, cyclobutyl, cyclopentyl or cyclohexyl.

Aspect 16. The compound of anyone of the preceding Aspects, wherein R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5f};
R^{5d} and R^{5f} are each independently selected from hydrogen, —F, —Cl, —Br, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl;
more preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl.

Aspect 17. The compound of anyone of the preceding Aspects, wherein R⁵, R⁶ and R⁷ are each independently selected from H, —F, —Cl, —Br, —I, methyl, ethyl, propyl, butyl, pentyl, hexyl, heptyl or octyl; and/or
R⁸ is selected from —F, —Cl, —Br or —I; and/or
R⁹ is selected from H;
preferably, R⁵, R⁶ and R⁷ are each independently selected from H, —F, —Cl, methyl, ethyl, propyl or butyl; and/or
R⁸ is selected from —F, —Cl; and/or
R⁹ is selected from H.

Aspect 18. The compound of anyone of the preceding Aspects, wherein R¹¹ is selected from H, methyl, ethyl, propyl, butyl, pentyl, hexyl, heptyl, octyl, —C₂₋₈alkenyl or —C₂₋₈alkynyl; wherein each of said methyl, ethyl, propyl, butyl, pentyl, hexyl, heptyl, octyl, —C₂₋₈alkenyl or —C₂₋₈alkynyl is optionally substituted with at least one substituent R^{11a},
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R¹¹ is selected from H, methyl, ethyl, propyl, butyl, pentyl, hexyl, heptyl, octyl, —C₂₋₈alkenyl or —C₂₋₈alkynyl;
more preferably, R¹¹ is selected from H, methyl, ethyl, propyl or butyl;
even more preferably, R¹¹ is H.

Aspect 19. The compound of anyone of the preceding Aspects, wherein the compound is selected from

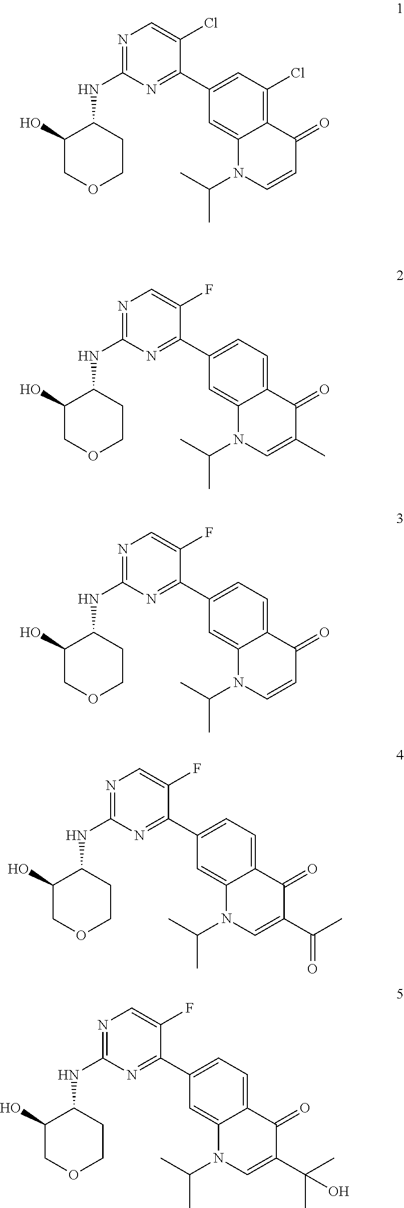

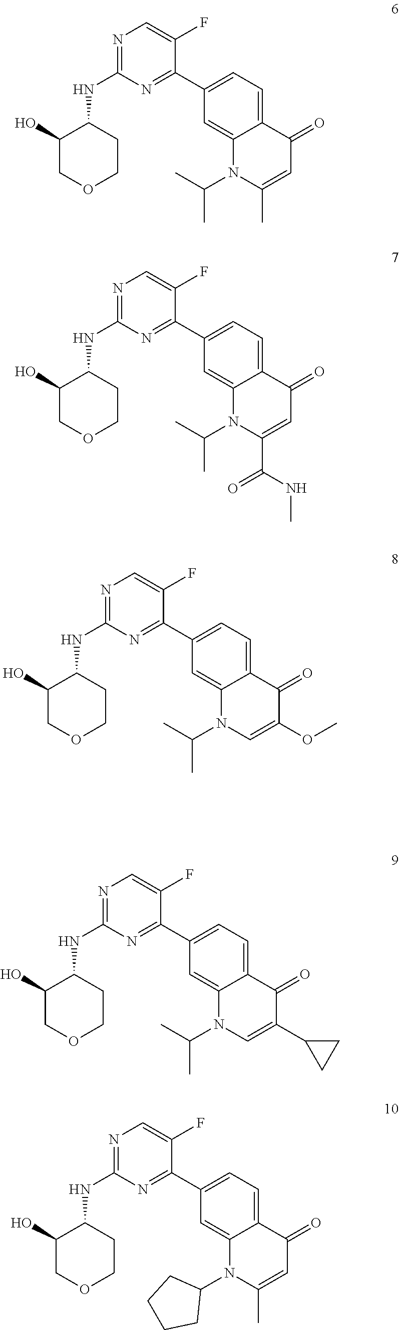 [SMILES: Cc1cc(=O)c2ccc(-c3nc(N[C@@H]4CCOC[C@H]4O)ncc3F)cc2n1C(C)C ; CNC(=O)c1cc(=O)c2ccc(-c3nc(N[C@@H]4CCOC[C@H]4O)ncc3F)cc2n1C(C)C ; COc1cn(C(C)C)c2cc(-c3nc(N[C@@H]4CCOC[C@H]4O)ncc3F)ccc2c1=O ; CC(C)n1cc(C2CC2)c(=O)c2ccc(-c3nc(N[C@@H]4CCOC[C@H]4O)ncc3F)cc21 ; Cc1cc(=O)c2ccc(-c3nc(N[C@@H]4CCOC[C@H]4O)ncc3F)cc2n1C1CCCC1]

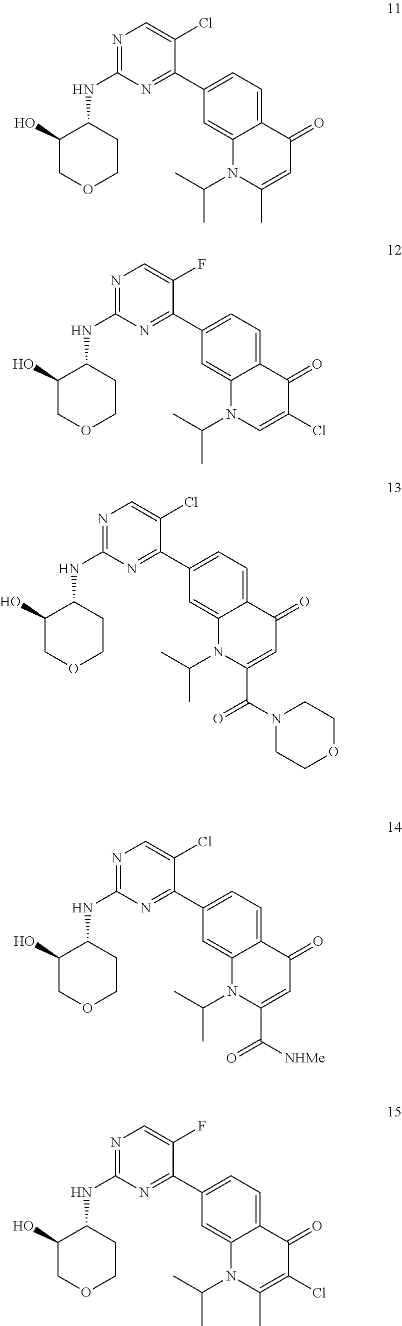

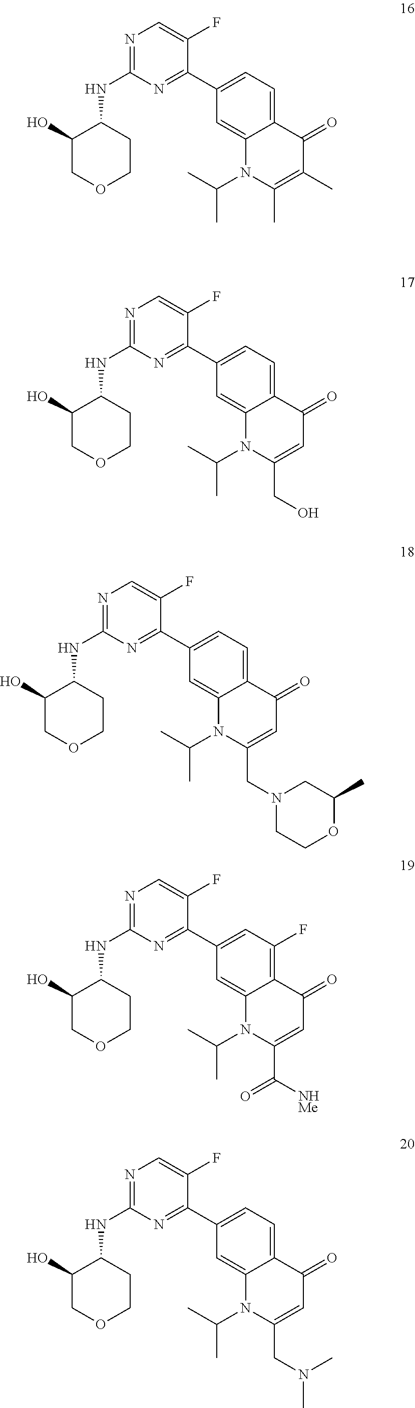 [SMILES: Cc1c(C)n(C(C)C)c2cc(-c3nc(N[C@@H]4CCOC[C@H]4O)ncc3F)ccc2c1=O ; CC(C)n1c(CO)cc(=O)c2ccc(-c3nc(N[C@@H]4CCOC[C@H]4O)ncc3F)cc21 ; CC(C)n1c(CN2CCO[C@H](C)C2)cc(=O)c2ccc(-c3nc(N[C@@H]4CCOC[C@H]4O)ncc3F)cc21 ; CNC(=O)c1cc(=O)c2c(F)cc(-c3nc(N[C@@H]4CCOC[C@H]4O)ncc3F)cc2n1C(C)C ; CC(C)n1c(CN(C)C)cc(=O)c2ccc(-c3nc(N[C@@H]4CCOC[C@H]4O)ncc3F)cc21]

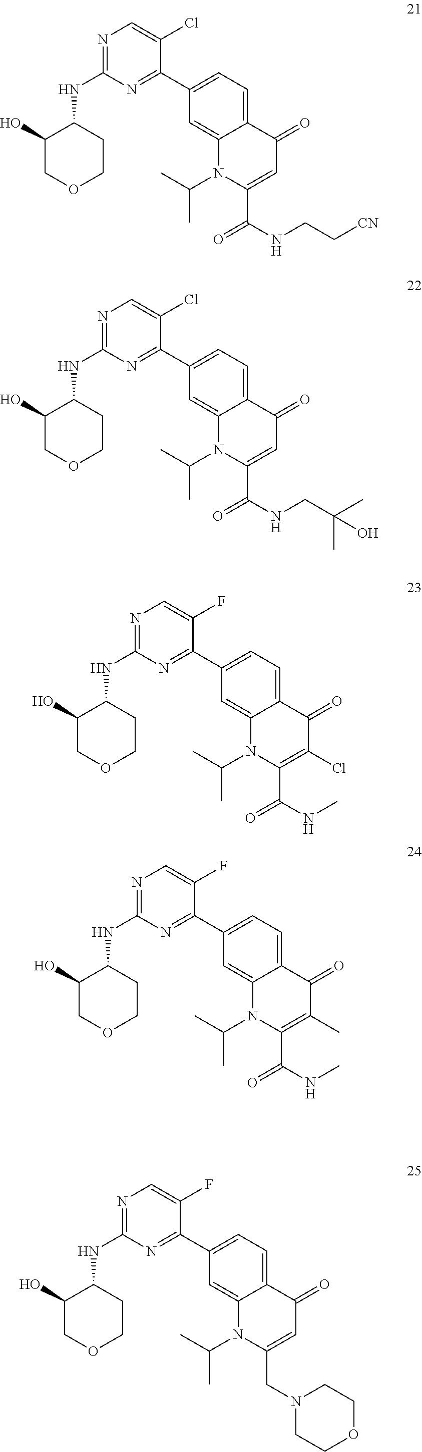 [SMILES: CC(C)n1c(C(=O)NCCC#N)cc(=O)c2ccc(-c3nc(N[C@@H]4CCOC[C@H]4O)ncc3Cl)cc21 ; CC(C)n1c(C(=O)NCC(C)(C)O)cc(=O)c2ccc(-c3nc(N[C@@H]4CCOC[C@H]4O)ncc3Cl)cc21 ; CNC(=O)c1c(Cl)c(=O)c2ccc(-c3nc(N[C@@H]4CCOC[C@H]4O)ncc3F)cc2n1C(C)C ; CNC(=O)c1c(C)c(=O)c2ccc(-c3nc(N[C@@H]4CCOC[C@H]4O)ncc3F)cc2n1C(C)C ; CC(C)n1c(CN2CCOCC2)cc(=O)c2ccc(-c3nc(N[C@@H]4CCOC[C@H]4O)ncc3F)cc21]

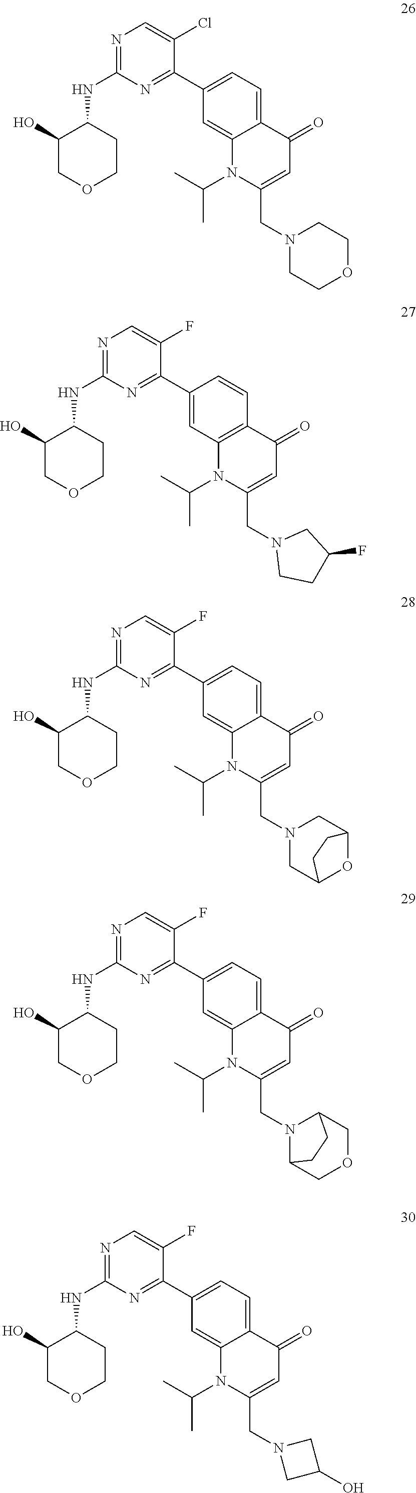 [SMILES: CC(C)n1c(CN2CCOCC2)cc(=O)c2ccc(-c3nc(N[C@@H]4CCOC[C@H]4O)ncc3Cl)cc21 ; CC(C)n1c(CN2CC[C@H](F)C2)cc(=O)c2ccc(-c3nc(N[C@@H]4CCOC[C@H]4O)ncc3F)cc21 ; CC(C)n1c(CN2CC3CCC(C2)O3)cc(=O)c2ccc(-c3nc(N[C@@H]4CCOC[C@H]4O)ncc3F)cc21 ; CC(C)n1c(CN2C3CCC2COC3)cc(=O)c2ccc(-c3nc(N[C@@H]4CCOC[C@H]4O)ncc3F)cc21 ; CC(C)n1c(CN2CC(O)C2)cc(=O)c2ccc(-c3nc(N[C@@H]4CCOC[C@H]4O)ncc3F)cc21]

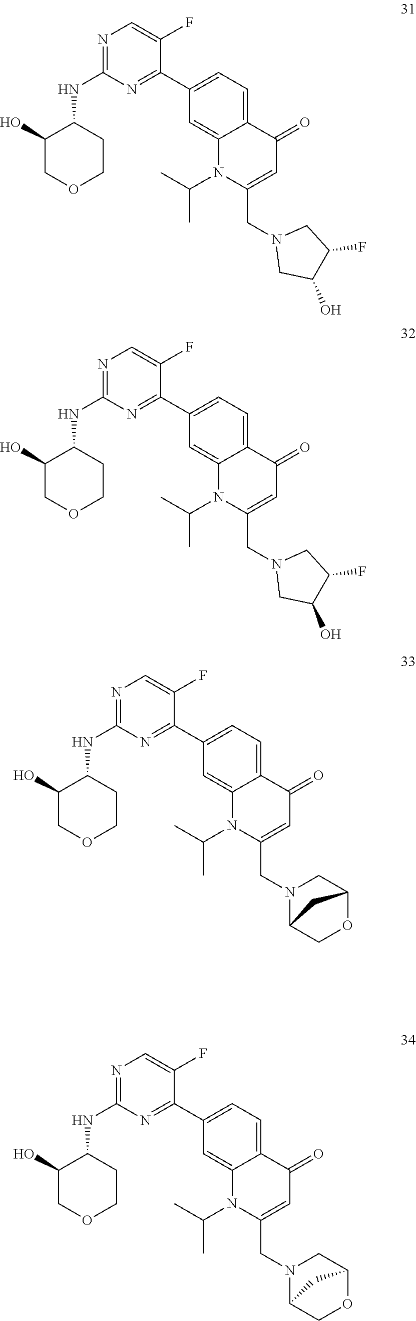 [SMILES: CC(C)n1c(CN2C[C@@H](O)[C@@H](F)C2)cc(=O)c2ccc(-c3nc(N[C@@H]4CCOC[C@H]4O)ncc3F)cc21 ; CC(C)n1c(CN2C[C@H](O)[C@@H](F)C2)cc(=O)c2ccc(-c3nc(N[C@@H]4CCOC[C@H]4O)ncc3F)cc21 ; CC(C)n1c(CN2C[C@H]3C[C@@H]2CO3)cc(=O)c2ccc(-c3nc(N[C@@H]4CCOC[C@H]4O)ncc3F)cc21 ; CC(C)n1c(CN2C[C@@H]3C[C@H]2CO3)cc(=O)c2ccc(-c3nc(N[C@@H]4CCOC[C@H]4O)ncc3F)cc21]

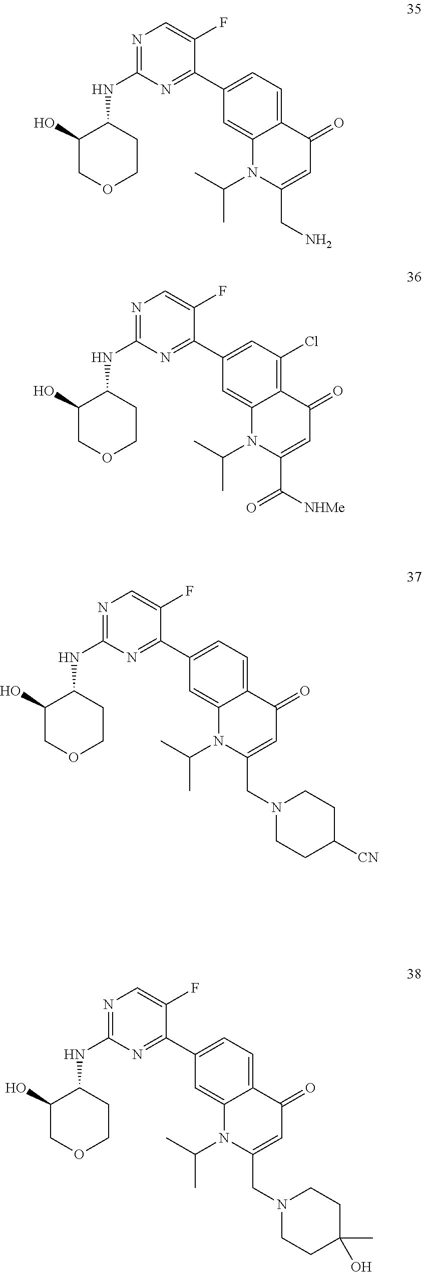 [SMILES: CC(C)n1c(CN)cc(=O)c2ccc(-c3nc(N[C@@H]4CCOC[C@H]4O)ncc3F)cc21 ; CNC(=O)c1cc(=O)c2c(Cl)cc(-c3nc(N[C@@H]4CCOC[C@H]4O)ncc3F)cc2n1C(C)C ; CC(C)n1c(CN2CCC(C#N)CC2)cc(=O)c2ccc(-c3nc(N[C@@H]4CCOC[C@H]4O)ncc3F)cc21 ; CC(C)n1c(CN2CCC(C)(O)CC2)cc(=O)c2ccc(-c3nc(N[C@@H]4CCOC[C@H]4O)ncc3F)cc21]

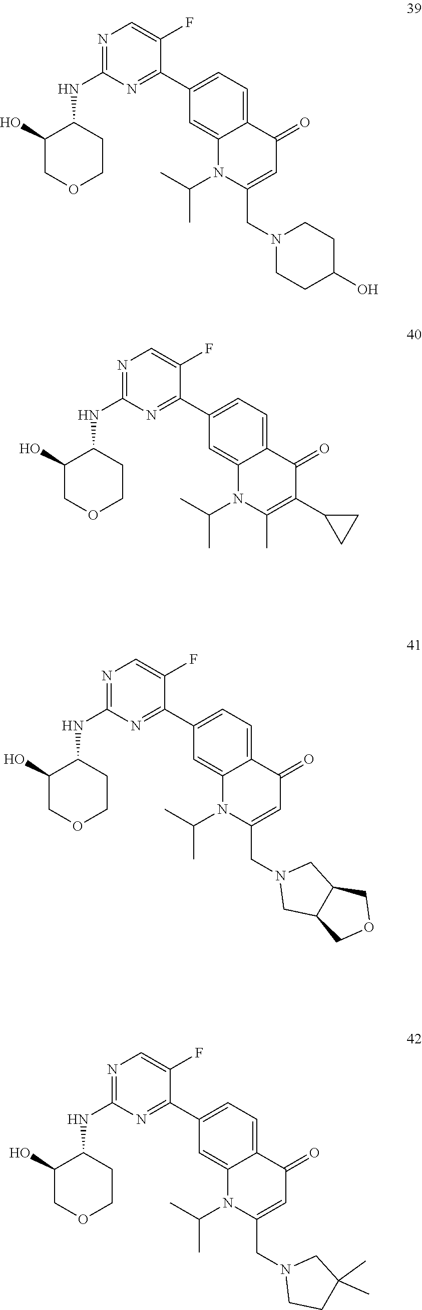 [SMILES: CC(C)n1c(CN2CCC(O)CC2)cc(=O)c2ccc(-c3nc(N[C@@H]4CCOC[C@H]4O)ncc3F)cc21 ; Cc1c(C2CC2)c(=O)c2ccc(-c3nc(N[C@@H]4CCOC[C@H]4O)ncc3F)cc2n1C(C)C ; CC(C)n1c(CN2C[C@H]3COC[C@H]3C2)cc(=O)c2ccc(-c3nc(N[C@@H]4CCOC[C@H]4O)ncc3F)cc21 ; CC(C)n1c(CN2CCC(C)(C)C2)cc(=O)c2ccc(-c3nc(N[C@@H]4CCOC[C@H]4O)ncc3F)cc21]

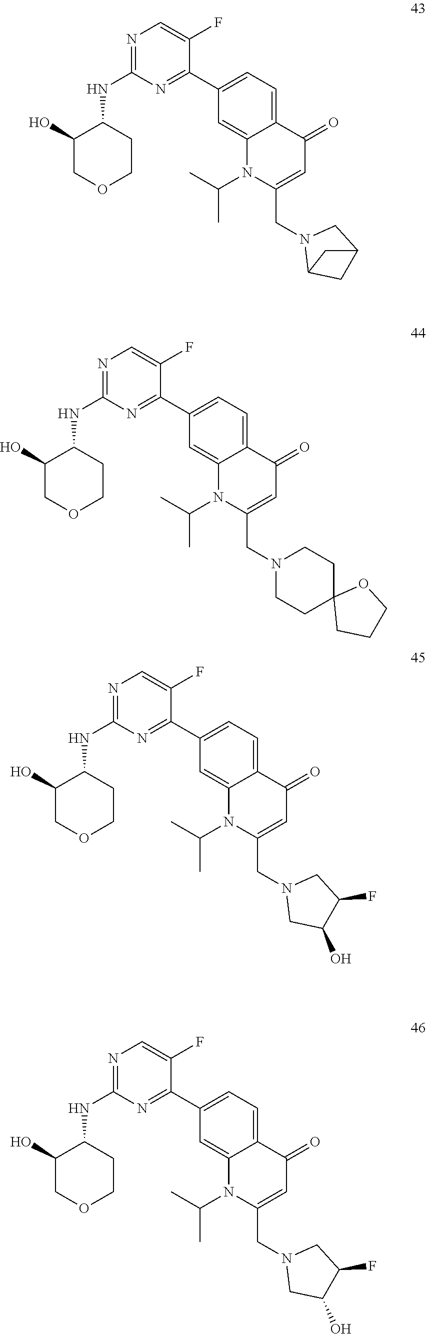 [SMILES: CC(C)n1c(CN2CC3CC2C3)cc(=O)c2ccc(-c3nc(N[C@@H]4CCOC[C@H]4O)ncc3F)cc21 ; CC(C)n1c(CN2CCC3(CCCO3)CC2)cc(=O)c2ccc(-c3nc(N[C@@H]4CCOC[C@H]4O)ncc3F)cc21 ; CC(C)n1c(CN2C[C@@H](F)[C@@H](O)C2)cc(=O)c2ccc(-c3nc(N[C@@H]4CCOC[C@H]4O)ncc3F)cc21 ; CC(C)n1c(CN2C[C@@H](O)[C@H](F)C2)cc(=O)c2ccc(-c3nc(N[C@@H]4CCOC[C@H]4O)ncc3F)cc21]

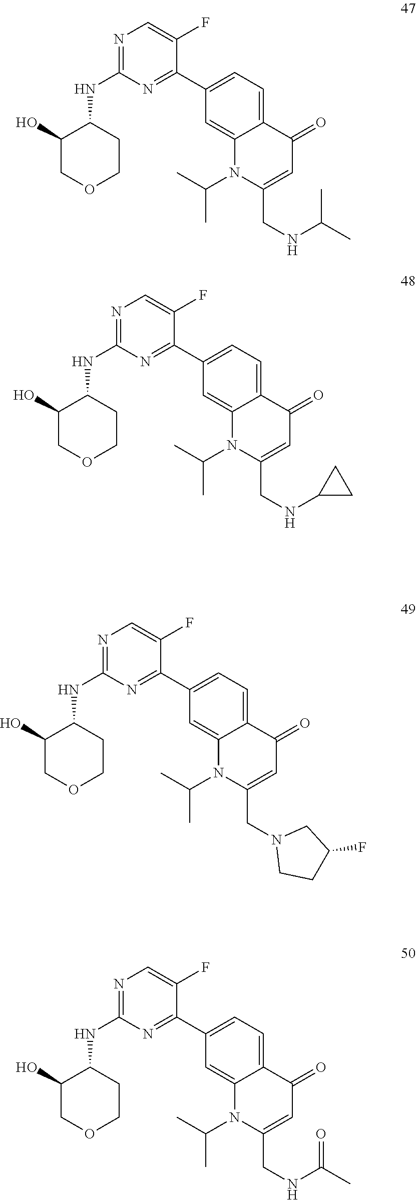 [SMILES: CC(C)NCc1cc(=O)c2ccc(-c3nc(N[C@@H]4CCOC[C@H]4O)ncc3F)cc2n1C(C)C ; CC(C)n1c(CNC2CC2)cc(=O)c2ccc(-c3nc(N[C@@H]4CCOC[C@H]4O)ncc3F)cc21 ; CC(C)n1c(CN2CC[C@@H](F)C2)cc(=O)c2ccc(-c3nc(N[C@@H]4CCOC[C@H]4O)ncc3F)cc21 ; CC(=O)NCc1cc(=O)c2ccc(-c3nc(N[C@@H]4CCOC[C@H]4O)ncc3F)cc2n1C(C)C]

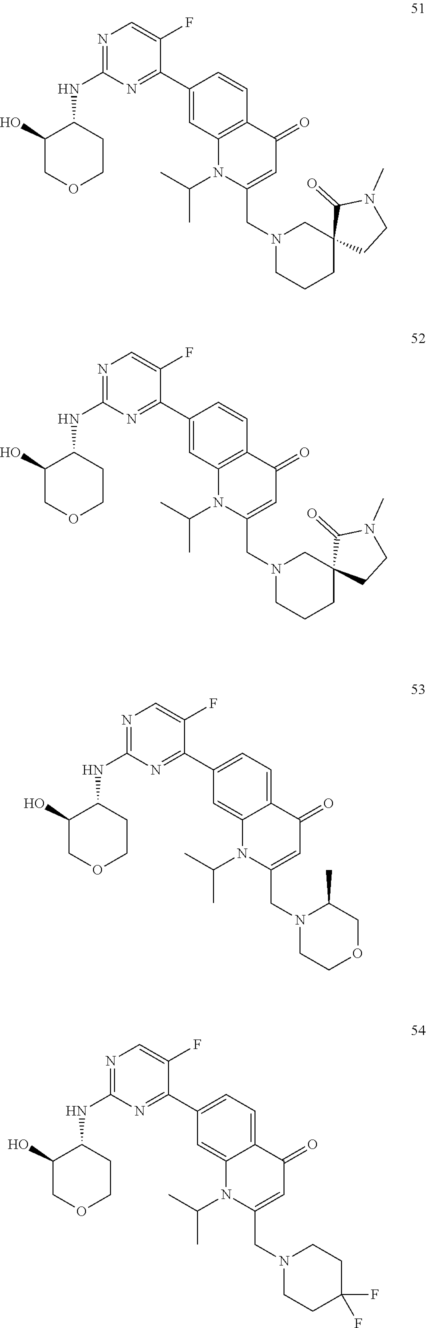 [SMILES: CC(C)n1c(CN2CCC[C@@]3(CCN(C)C3=O)C2)cc(=O)c2ccc(-c3nc(N[C@@H]4CCOC[C@H]4O)ncc3F)cc21 ; CC(C)n1c(CN2CCC[C@]3(CCN(C)C3=O)C2)cc(=O)c2ccc(-c3nc(N[C@@H]4CCOC[C@H]4O)ncc3F)cc21 ; CC(C)n1c(CN2CCOC[C@@H]2C)cc(=O)c2ccc(-c3nc(N[C@@H]4CCOC[C@H]4O)ncc3F)cc21 ; CC(C)n1c(CN2CCC(F)(F)CC2)cc(=O)c2ccc(-c3nc(N[C@@H]4CCOC[C@H]4O)ncc3F)cc21]

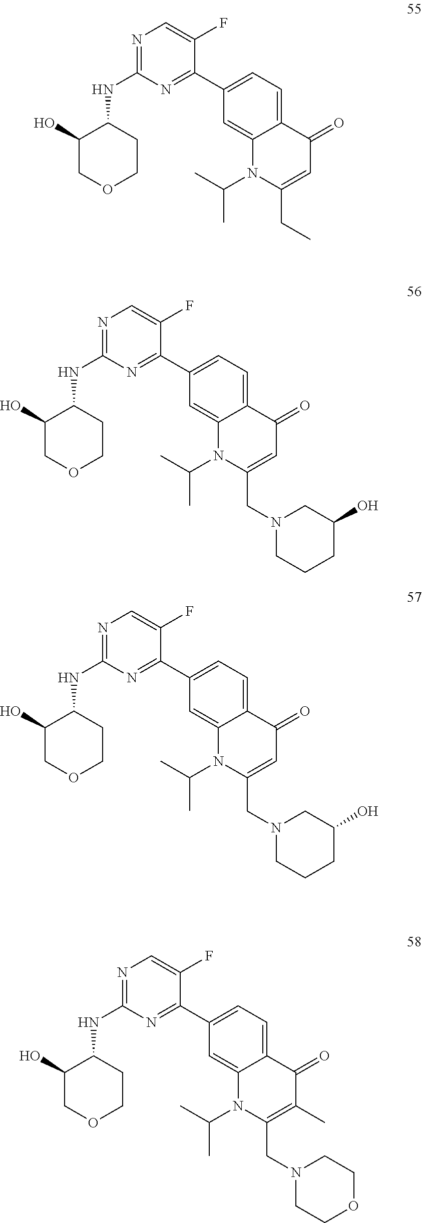 [SMILES: CCc1cc(=O)c2ccc(-c3nc(N[C@@H]4CCOC[C@H]4O)ncc3F)cc2n1C(C)C ; CC(C)n1c(CN2CCC[C@H](O)C2)cc(=O)c2ccc(-c3nc(N[C@@H]4CCOC[C@H]4O)ncc3F)cc21 ; CC(C)n1c(CN2CCC[C@@H](O)C2)cc(=O)c2ccc(-c3nc(N[C@@H]4CCOC[C@H]4O)ncc3F)cc21 ; Cc1c(CN2CCOCC2)n(C(C)C)c2cc(-c3nc(N[C@@H]4CCOC[C@H]4O)ncc3F)ccc2c1=O]

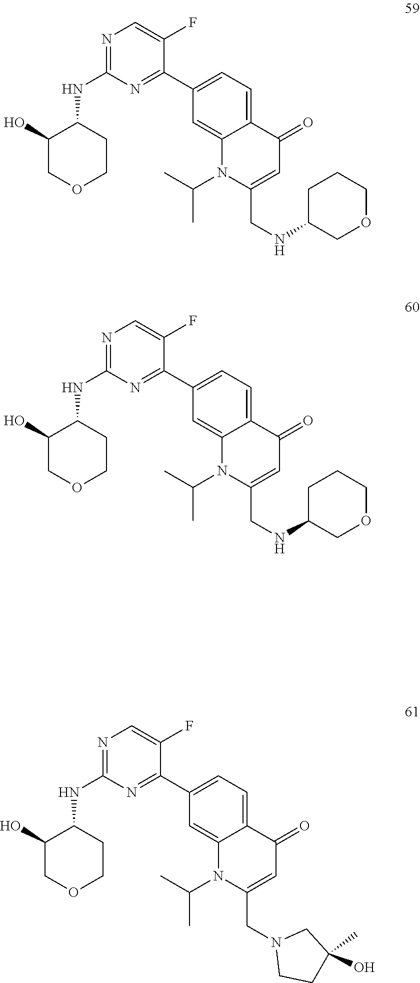 [SMILES: CC(C)n1c(CN[C@@H]2CCCOC2)cc(=O)c2ccc(-c3nc(N[C@@H]4CCOC[C@H]4O)ncc3F)cc21 ; CC(C)n1c(CN[C@H]2CCCOC2)cc(=O)c2ccc(-c3nc(N[C@@H]4CCOC[C@H]4O)ncc3F)cc21 ; CC(C)n1c(CN2CC[C@](C)(O)C2)cc(=O)c2ccc(-c3nc(N[C@@H]4CCOC[C@H]4O)ncc3F)cc21]

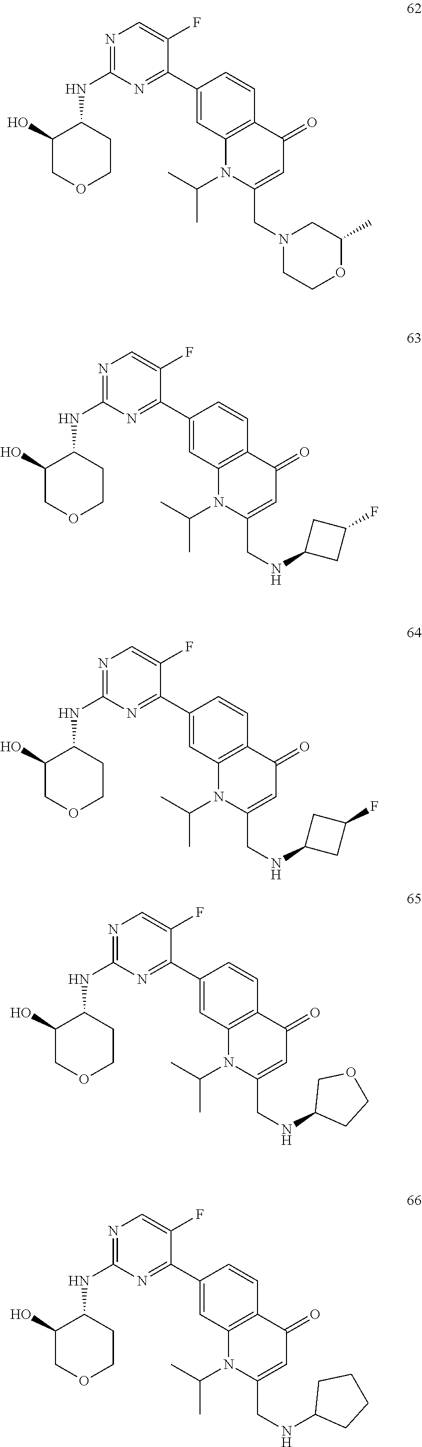 [SMILES: CC(C)n1c(CN2CCO[C@@H](C)C2)cc(=O)c2ccc(-c3nc(N[C@@H]4CCOC[C@H]4O)ncc3F)cc21 ; CC(C)n1c(CN[C@H]2C[C@H](F)C2)cc(=O)c2ccc(-c3nc(N[C@@H]4CCOC[C@H]4O)ncc3F)cc21 ; CC(C)n1c(CN[C@H]2C[C@@H](F)C2)cc(=O)c2ccc(-c3nc(N[C@@H]4CCOC[C@H]4O)ncc3F)cc21 ; CC(C)n1c(CN[C@@H]2CCOC2)cc(=O)c2ccc(-c3nc(N[C@@H]4CCOC[C@H]4O)ncc3F)cc21 ; CC(C)n1c(CNC2CCCC2)cc(=O)c2ccc(-c3nc(N[C@@H]4CCOC[C@H]4O)ncc3F)cc21]

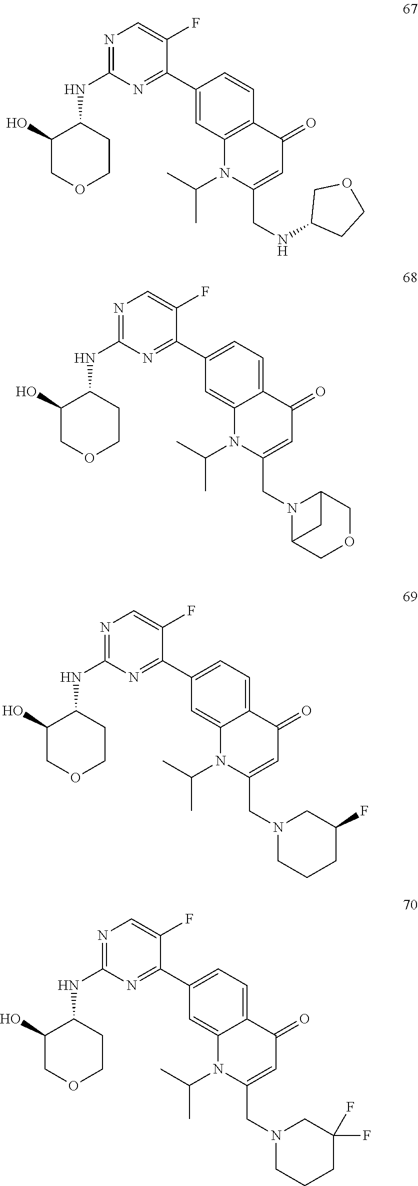 [SMILES: CC(C)n1c(CN[C@H]2CCOC2)cc(=O)c2ccc(-c3nc(N[C@@H]4CCOC[C@H]4O)ncc3F)cc21 ; CC(C)n1c(CN2C3COCC2C3)cc(=O)c2ccc(-c3nc(N[C@@H]4CCOC[C@H]4O)ncc3F)cc21 ; CC(C)n1c(CN2CCC[C@H](F)C2)cc(=O)c2ccc(-c3nc(N[C@@H]4CCOC[C@H]4O)ncc3F)cc21 ; CC(C)n1c(CN2CCCC(F)(F)C2)cc(=O)c2ccc(-c3nc(N[C@@H]4CCOC[C@H]4O)ncc3F)cc21]

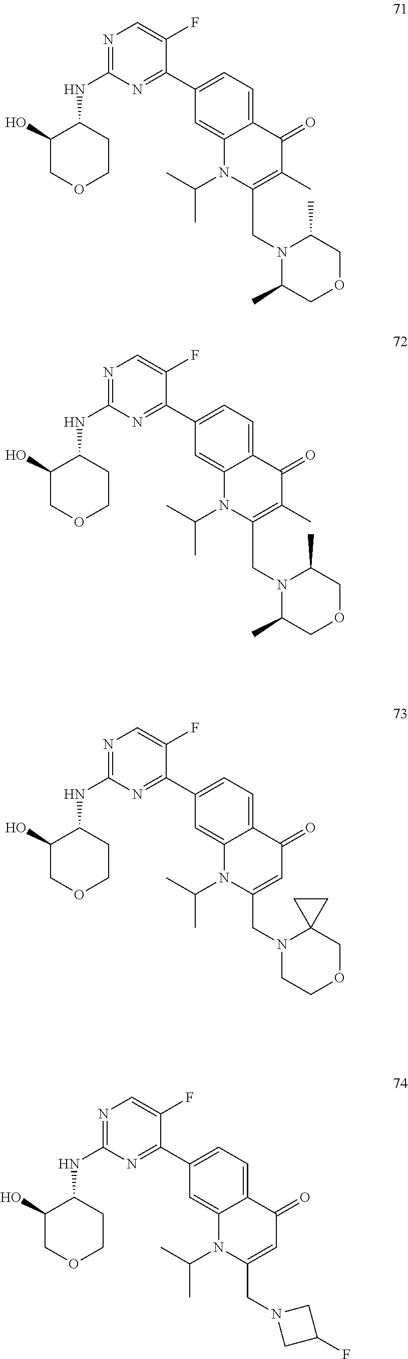 [SMILES: Cc1c(CN2[C@H](C)COC[C@H]2C)n(C(C)C)c2cc(-c3nc(N[C@@H]4CCOC[C@H]4O)ncc3F)ccc2c1=O ; Cc1c(CN2[C@H](C)COC[C@@H]2C)n(C(C)C)c2cc(-c3nc(N[C@@H]4CCOC[C@H]4O)ncc3F)ccc2c1=O ; CC(C)n1c(CN2CCOCC23CC3)cc(=O)c2ccc(-c3nc(N[C@@H]4CCOC[C@H]4O)ncc3F)cc21 ; CC(C)n1c(CN2CC(F)C2)cc(=O)c2ccc(-c3nc(N[C@@H]4CCOC[C@H]4O)ncc3F)cc21]

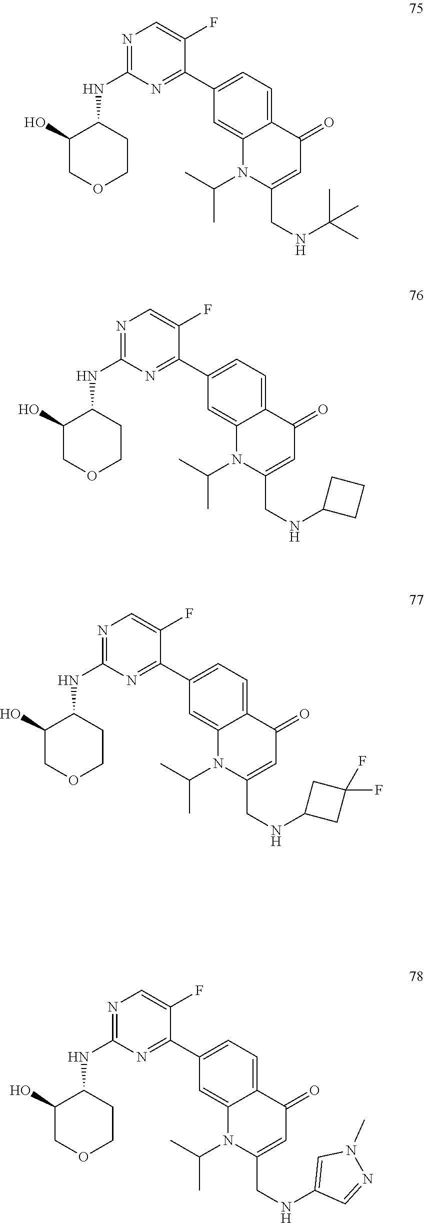 [SMILES: CC(C)n1c(CNC(C)(C)C)cc(=O)c2ccc(-c3nc(N[C@@H]4CCOC[C@H]4O)ncc3F)cc21 ; CC(C)n1c(CNC2CCC2)cc(=O)c2ccc(-c3nc(N[C@@H]4CCOC[C@H]4O)ncc3F)cc21 ; CC(C)n1c(CNC2CC(F)(F)C2)cc(=O)c2ccc(-c3nc(N[C@@H]4CCOC[C@H]4O)ncc3F)cc21 ; CC(C)n1c(CNc2cnn(C)c2)cc(=O)c2ccc(-c3nc(N[C@@H]4CCOC[C@H]4O)ncc3F)cc21]

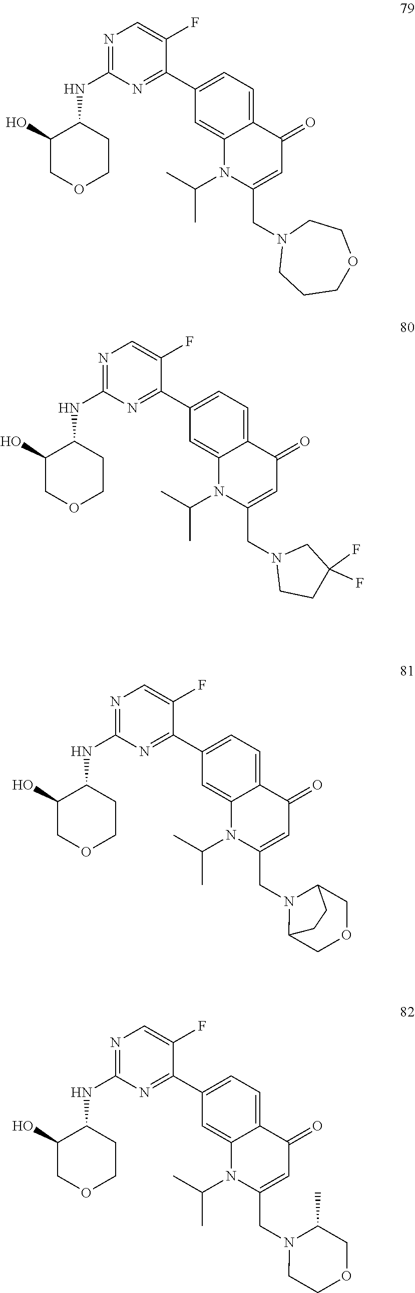 [SMILES: CC(C)n1c(CN2CCCOCC2)cc(=O)c2ccc(-c3nc(N[C@@H]4CCOC[C@H]4O)ncc3F)cc21 ; CC(C)n1c(CN2CCC(F)(F)C2)cc(=O)c2ccc(-c3nc(N[C@@H]4CCOC[C@H]4O)ncc3F)cc21 ; CC(C)n1c(CN2C3CCC2COC3)cc(=O)c2ccc(-c3nc(N[C@@H]4CCOC[C@H]4O)ncc3F)cc21 ; CC(C)n1c(CN2CCOC[C@H]2C)cc(=O)c2ccc(-c3nc(N[C@@H]4CCOC[C@H]4O)ncc3F)cc21]

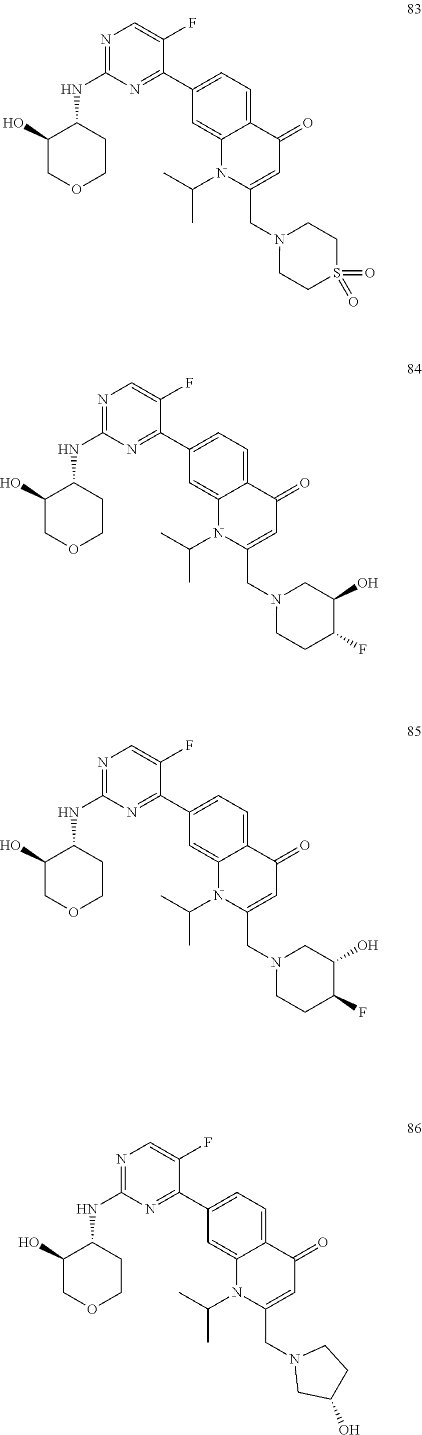 [SMILES: CC(C)n1c(CN2CCS(=O)(=O)CC2)cc(=O)c2ccc(-c3nc(N[C@@H]4CCOC[C@H]4O)ncc3F)cc21 ; CC(C)n1c(CN2CC[C@@H](F)[C@H](O)C2)cc(=O)c2ccc(-c3nc(N[C@@H]4CCOC[C@H]4O)ncc3F)cc21 ; CC(C)n1c(CN2CC[C@H](F)[C@@H](O)C2)cc(=O)c2ccc(-c3nc(N[C@@H]4CCOC[C@H]4O)ncc3F)cc21 ; CC(C)n1c(CN2CC[C@H](O)C2)cc(=O)c2ccc(-c3nc(N[C@@H]4CCOC[C@H]4O)ncc3F)cc21]

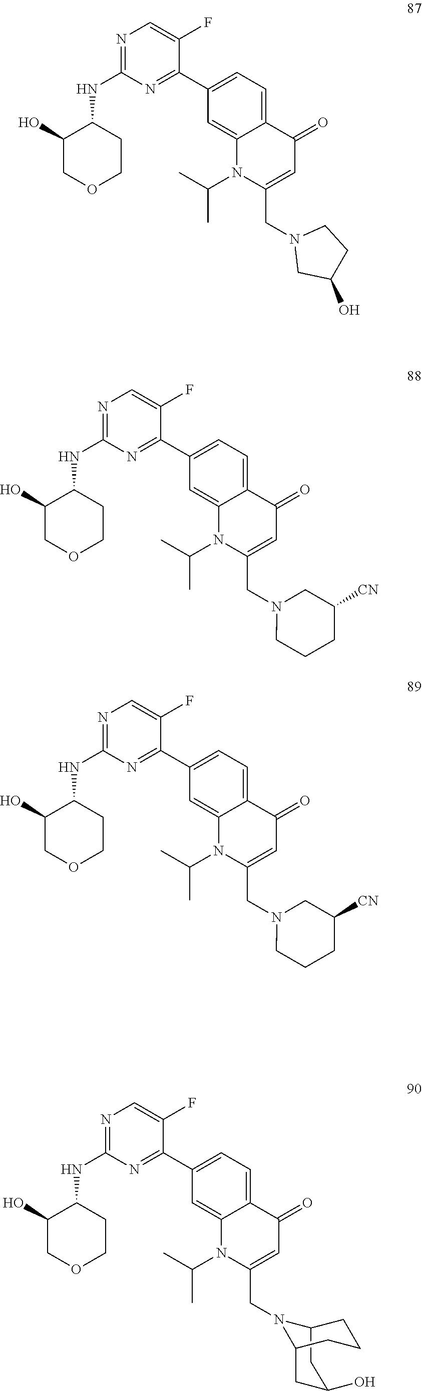 [SMILES: CC(C)n1c(CN2CC[C@@H](O)C2)cc(=O)c2ccc(-c3nc(N[C@@H]4CCOC[C@H]4O)ncc3F)cc21 ; CC(C)n1c(CN2CCC[C@@H](C#N)C2)cc(=O)c2ccc(-c3nc(N[C@@H]4CCOC[C@H]4O)ncc3F)cc21 ; CC(C)n1c(CN2CCC[C@H](C#N)C2)cc(=O)c2ccc(-c3nc(N[C@@H]4CCOC[C@H]4O)ncc3F)cc21 ; CC(C)n1c(CN2C3CCCC2CC(O)C3)cc(=O)c2ccc(-c3nc(N[C@@H]4CCOC[C@H]4O)ncc3F)cc21]

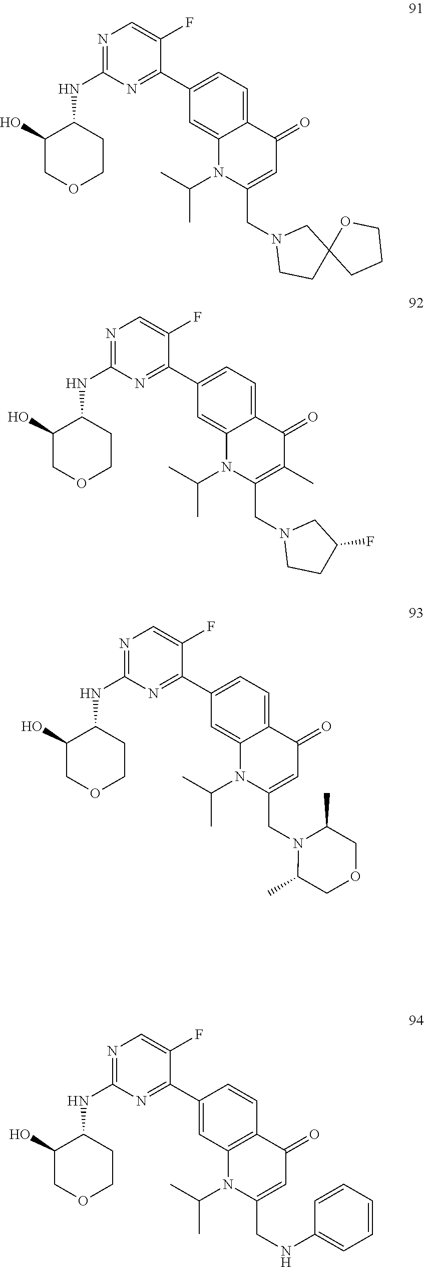 [SMILES: CC(C)n1c(CN2CCC3(CCCO3)C2)cc(=O)c2ccc(-c3nc(N[C@@H]4CCOC[C@H]4O)ncc3F)cc21 ; Cc1c(CN2CC[C@@H](F)C2)n(C(C)C)c2cc(-c3nc(N[C@@H]4CCOC[C@H]4O)ncc3F)ccc2c1=O ; CC(C)n1c(CN2[C@@H](C)COC[C@@H]2C)cc(=O)c2ccc(-c3nc(N[C@@H]4CCOC[C@H]4O)ncc3F)cc21 ; CC(C)n1c(CNc2ccccc2)cc(=O)c2ccc(-c3nc(N[C@@H]4CCOC[C@H]4O)ncc3F)cc21]

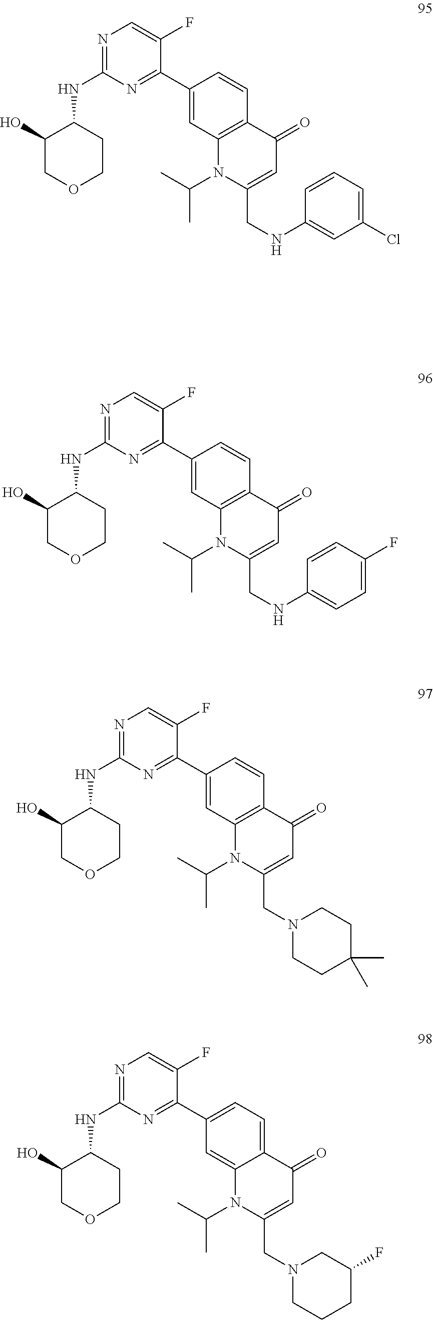 [SMILES: CC(C)n1c(CNc2cccc(Cl)c2)cc(=O)c2ccc(-c3nc(N[C@@H]4CCOC[C@H]4O)ncc3F)cc21 ; CC(C)n1c(CNc2ccc(F)cc2)cc(=O)c2ccc(-c3nc(N[C@@H]4CCOC[C@H]4O)ncc3F)cc21 ; CC(C)n1c(CN2CCC(C)(C)CC2)cc(=O)c2ccc(-c3nc(N[C@@H]4CCOC[C@H]4O)ncc3F)cc21 ; CC(C)n1c(CN2CCC[C@@H](F)C2)cc(=O)c2ccc(-c3nc(N[C@@H]4CCOC[C@H]4O)ncc3F)cc21]

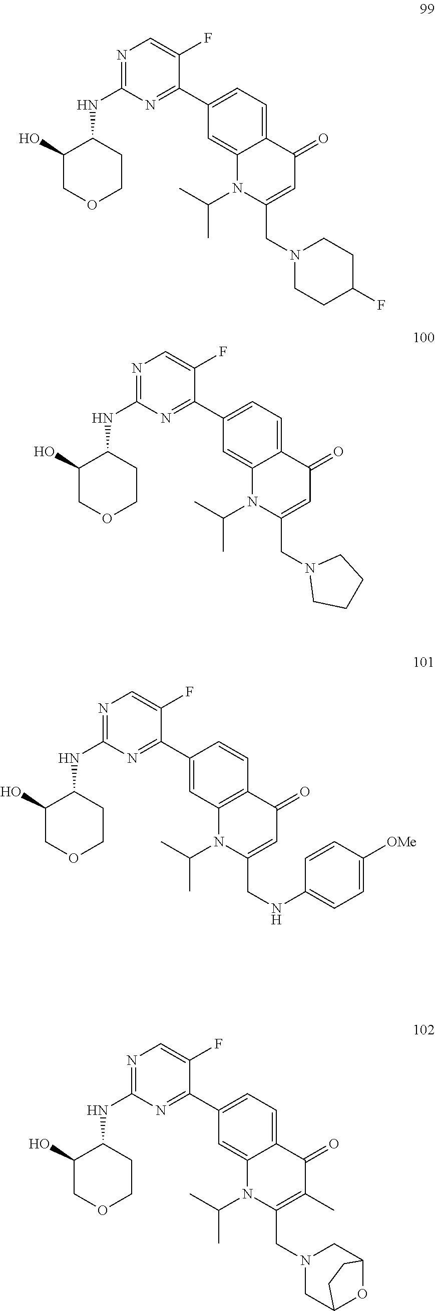 [SMILES: CC(C)n1c(CN2CCC(F)CC2)cc(=O)c2ccc(-c3nc(N[C@@H]4CCOC[C@H]4O)ncc3F)cc21 ; CC(C)n1c(CN2CCCC2)cc(=O)c2ccc(-c3nc(N[C@@H]4CCOC[C@H]4O)ncc3F)cc21 ; COc1ccc(NCc2cc(=O)c3ccc(-c4nc(N[C@@H]5CCOC[C@H]5O)ncc4F)cc3n2C(C)C)cc1 ; Cc1c(CN2CC3CCC(C2)O3)n(C(C)C)c2cc(-c3nc(N[C@@H]4CCOC[C@H]4O)ncc3F)ccc2c1=O]

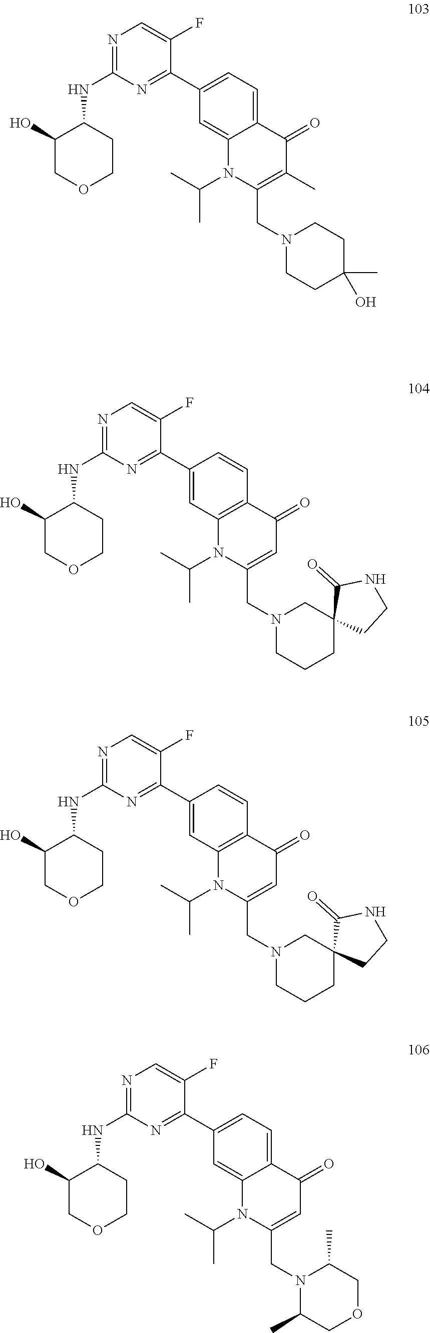 [SMILES: Cc1c(CN2CCC(C)(O)CC2)n(C(C)C)c2cc(-c3nc(N[C@@H]4CCOC[C@H]4O)ncc3F)ccc2c1=O ; CC(C)n1c(CN2CCC[C@@]3(CCNC3=O)C2)cc(=O)c2ccc(-c3nc(N[C@@H]4CCOC[C@H]4O)ncc3F)cc21 ; CC(C)n1c(CN2CCC[C@]3(CCNC3=O)C2)cc(=O)c2ccc(-c3nc(N[C@@H]4CCOC[C@H]4O)ncc3F)cc21 ; CC(C)n1c(CN2[C@H](C)COC[C@H]2C)cc(=O)c2ccc(-c3nc(N[C@@H]4CCOC[C@H]4O)ncc3F)cc21]

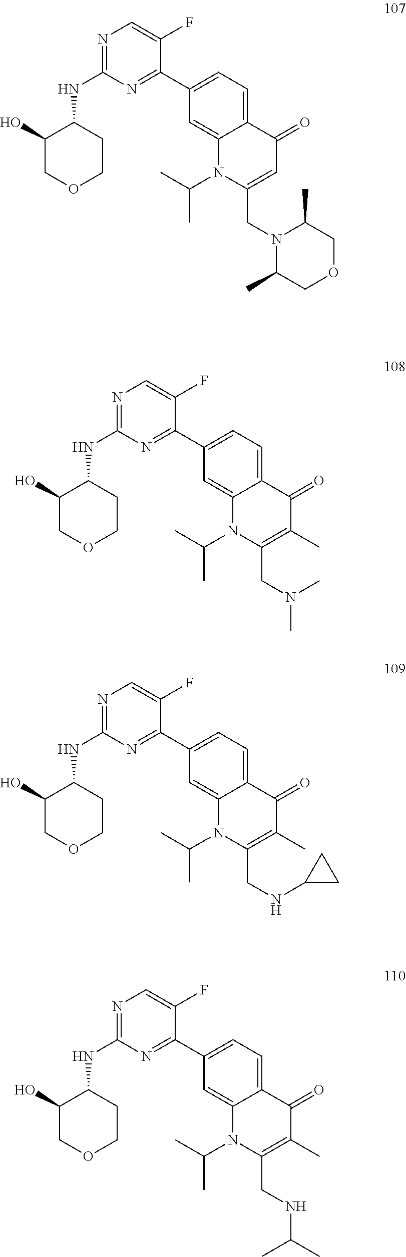 [SMILES: CC(C)n1c(CN2[C@H](C)COC[C@@H]2C)cc(=O)c2ccc(-c3nc(N[C@@H]4CCOC[C@H]4O)ncc3F)cc21 ; Cc1c(CN(C)C)n(C(C)C)c2cc(-c3nc(N[C@@H]4CCOC[C@H]4O)ncc3F)ccc2c1=O ; Cc1c(CNC2CC2)n(C(C)C)c2cc(-c3nc(N[C@@H]4CCOC[C@H]4O)ncc3F)ccc2c1=O ; Cc1c(CNC(C)C)n(C(C)C)c2cc(-c3nc(N[C@@H]4CCOC[C@H]4O)ncc3F)ccc2c1=O]

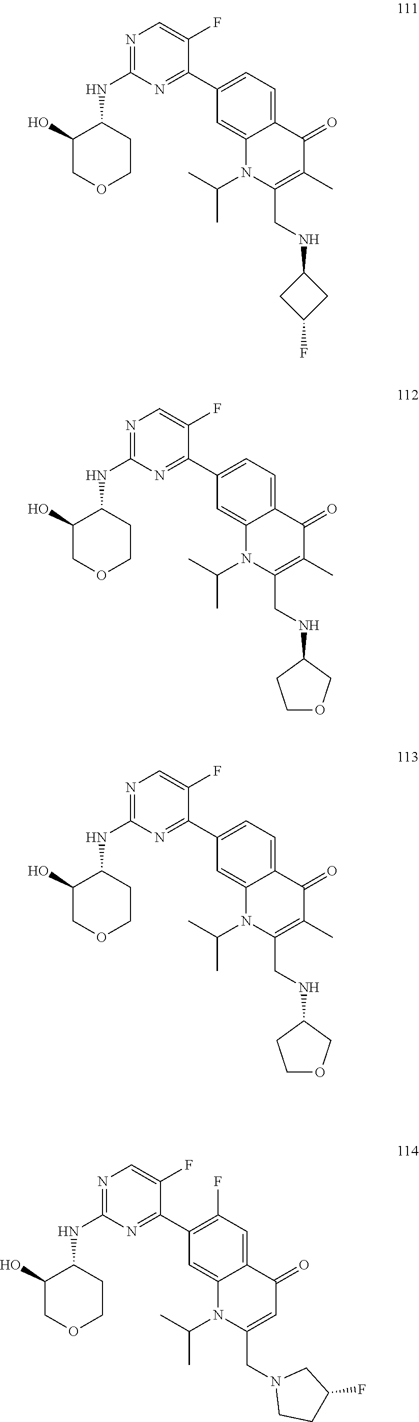 [SMILES: Cc1c(CN[C@H]2C[C@H](F)C2)n(C(C)C)c2cc(-c3nc(N[C@@H]4CCOC[C@H]4O)ncc3F)ccc2c1=O ; Cc1c(CN[C@@H]2CCOC2)n(C(C)C)c2cc(-c3nc(N[C@@H]4CCOC[C@H]4O)ncc3F)ccc2c1=O ; Cc1c(CN[C@H]2CCOC2)n(C(C)C)c2cc(-c3nc(N[C@@H]4CCOC[C@H]4O)ncc3F)ccc2c1=O ; CC(C)n1c(CN2CC[C@@H](F)C2)cc(=O)c2cc(F)c(-c3nc(N[C@@H]4CCOC[C@H]4O)ncc3F)cc21]

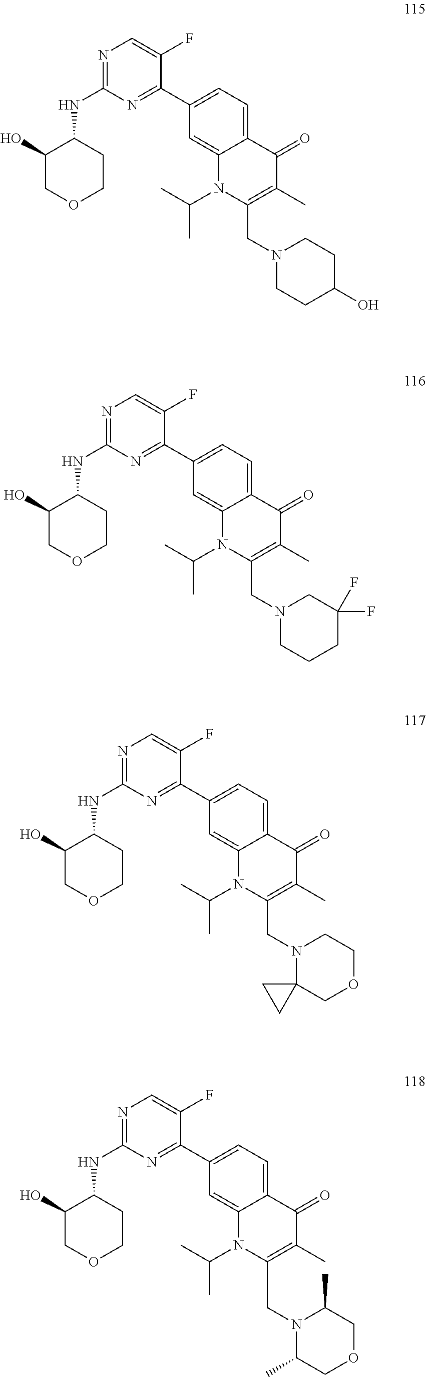 [SMILES: Cc1c(CN2CCC(O)CC2)n(C(C)C)c2cc(-c3nc(N[C@@H]4CCOC[C@H]4O)ncc3F)ccc2c1=O ; Cc1c(CN2CCCC(F)(F)C2)n(C(C)C)c2cc(-c3nc(N[C@@H]4CCOC[C@H]4O)ncc3F)ccc2c1=O ; Cc1c(CN2CCOCC23CC3)n(C(C)C)c2cc(-c3nc(N[C@@H]4CCOC[C@H]4O)ncc3F)ccc2c1=O ; Cc1c(CN2[C@@H](C)COC[C@@H]2C)n(C(C)C)c2cc(-c3nc(N[C@@H]4CCOC[C@H]4O)ncc3F)ccc2c1=O]

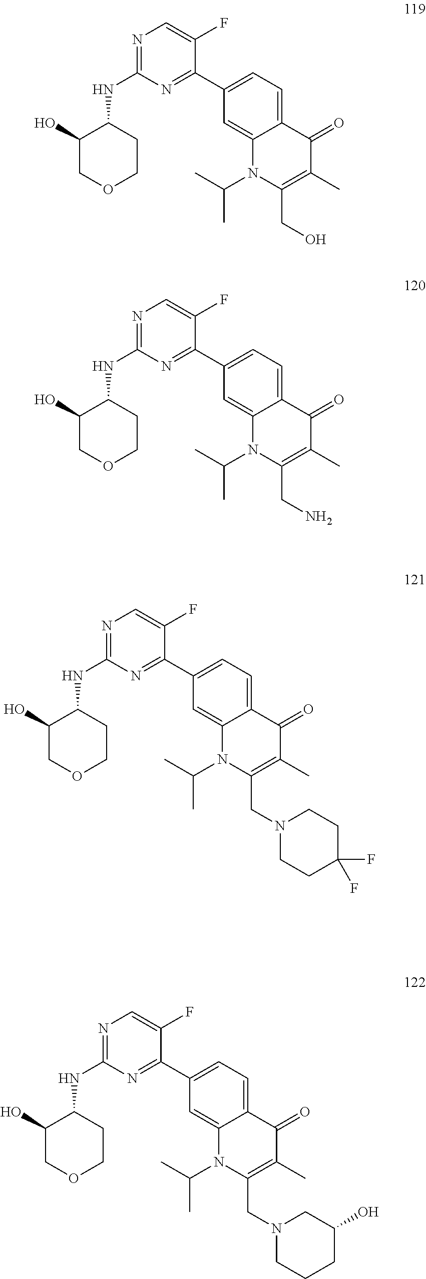 [SMILES: Cc1c(CO)n(C(C)C)c2cc(-c3nc(N[C@@H]4CCOC[C@H]4O)ncc3F)ccc2c1=O ; Cc1c(CN)n(C(C)C)c2cc(-c3nc(N[C@@H]4CCOC[C@H]4O)ncc3F)ccc2c1=O ; Cc1c(CN2CCC(F)(F)CC2)n(C(C)C)c2cc(-c3nc(N[C@@H]4CCOC[C@H]4O)ncc3F)ccc2c1=O ; Cc1c(CN2CCC[C@@H](O)C2)n(C(C)C)c2cc(-c3nc(N[C@@H]4CCOC[C@H]4O)ncc3F)ccc2c1=O]

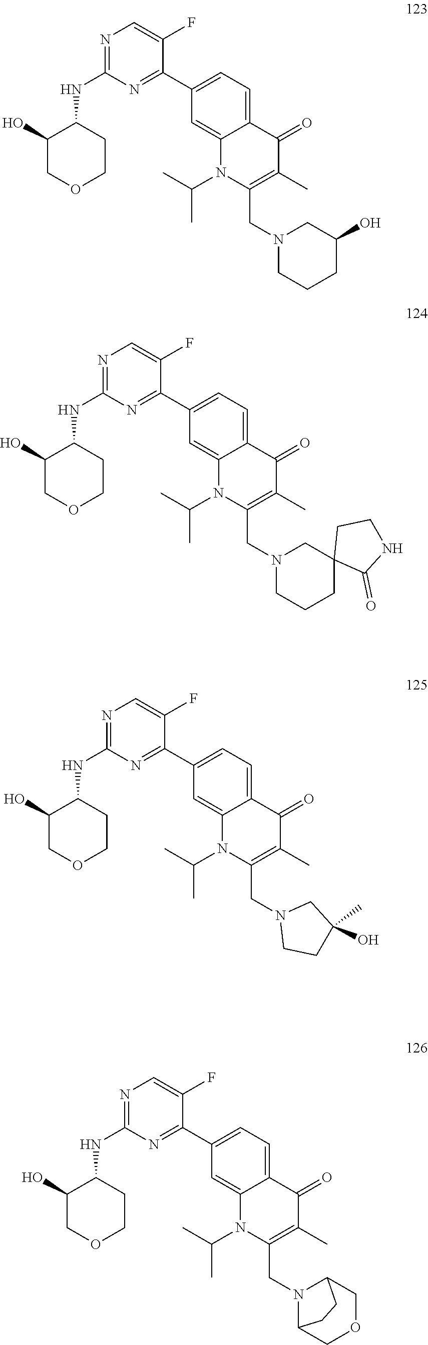 [SMILES: Cc1c(CN2CCC[C@H](O)C2)n(C(C)C)c2cc(-c3nc(N[C@@H]4CCOC[C@H]4O)ncc3F)ccc2c1=O ; Cc1c(CN2CCCC3(CCNC3=O)C2)n(C(C)C)c2cc(-c3nc(N[C@@H]4CCOC[C@H]4O)ncc3F)ccc2c1=O ; Cc1c(CN2CC[C@](C)(O)C2)n(C(C)C)c2cc(-c3nc(N[C@@H]4CCOC[C@H]4O)ncc3F)ccc2c1=O ; Cc1c(CN2C3CCC2COC3)n(C(C)C)c2cc(-c3nc(N[C@@H]4CCOC[C@H]4O)ncc3F)ccc2c1=O]

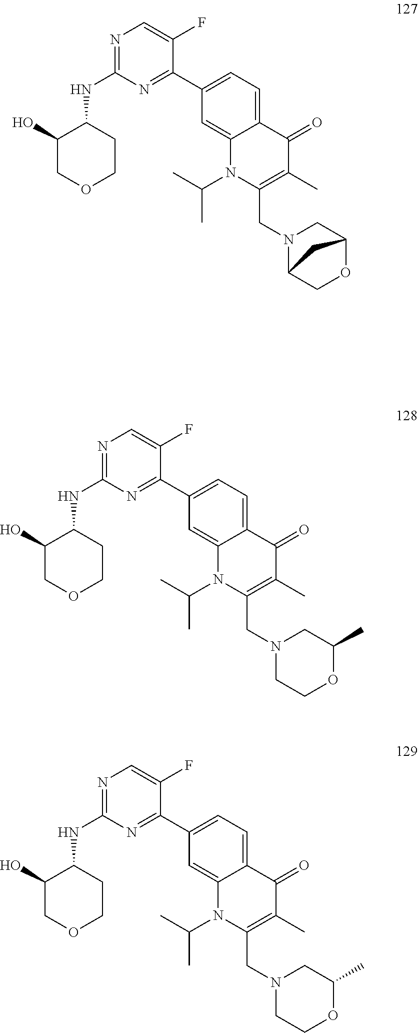 [SMILES: Cc1c(CN2C[C@H]3C[C@@H]2CO3)n(C(C)C)c2cc(-c3nc(N[C@@H]4CCOC[C@H]4O)ncc3F)ccc2c1=O ; Cc1c(CN2CCO[C@H](C)C2)n(C(C)C)c2cc(-c3nc(N[C@@H]4CCOC[C@H]4O)ncc3F)ccc2c1=O ; Cc1c(CN2CCO[C@@H](C)C2)n(C(C)C)c2cc(-c3nc(N[C@@H]4CCOC[C@H]4O)ncc3F)ccc2c1=O]

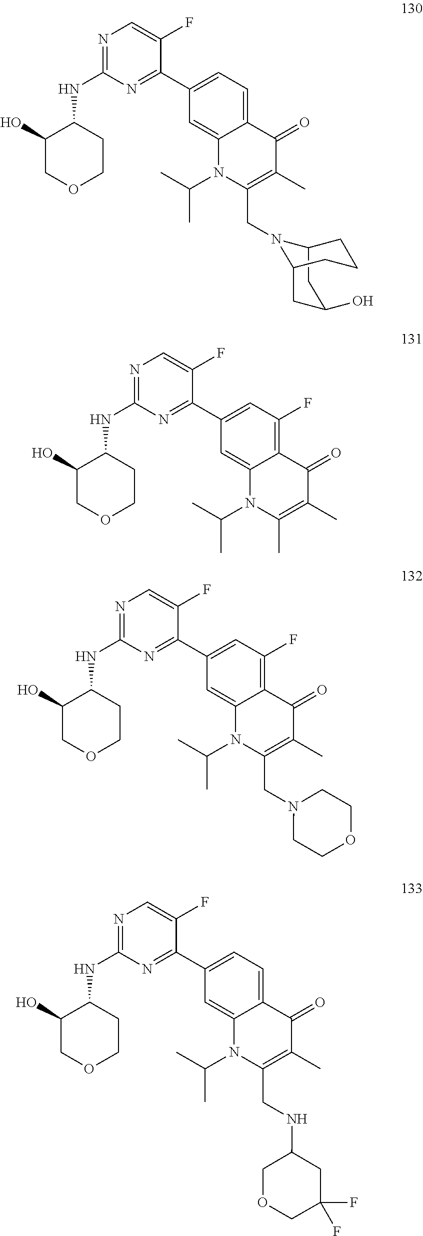

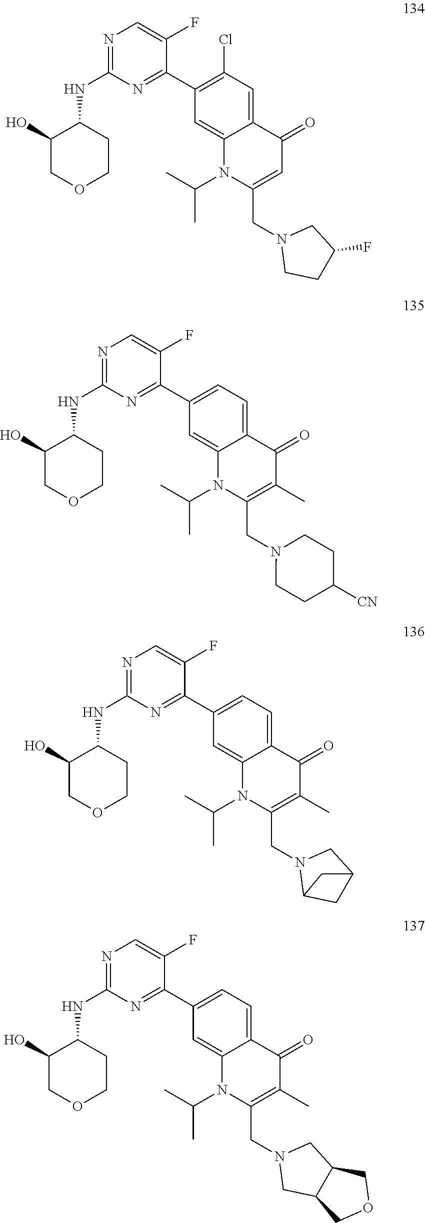 [SMILES: CC(C)n1c(CN2CC[C@@H](F)C2)cc(=O)c2cc(Cl)c(-c3nc(N[C@@H]4CCOC[C@H]4O)ncc3F)cc21 ; Cc1c(CN2CCC(C#N)CC2)n(C(C)C)c2cc(-c3nc(N[C@@H]4CCOC[C@H]4O)ncc3F)ccc2c1=O ; Cc1c(CN2CC3CC2C3)n(C(C)C)c2cc(-c3nc(N[C@@H]4CCOC[C@H]4O)ncc3F)ccc2c1=O ; Cc1c(CN2C[C@H]3COC[C@H]3C2)n(C(C)C)c2cc(-c3nc(N[C@@H]4CCOC[C@H]4O)ncc3F)ccc2c1=O]

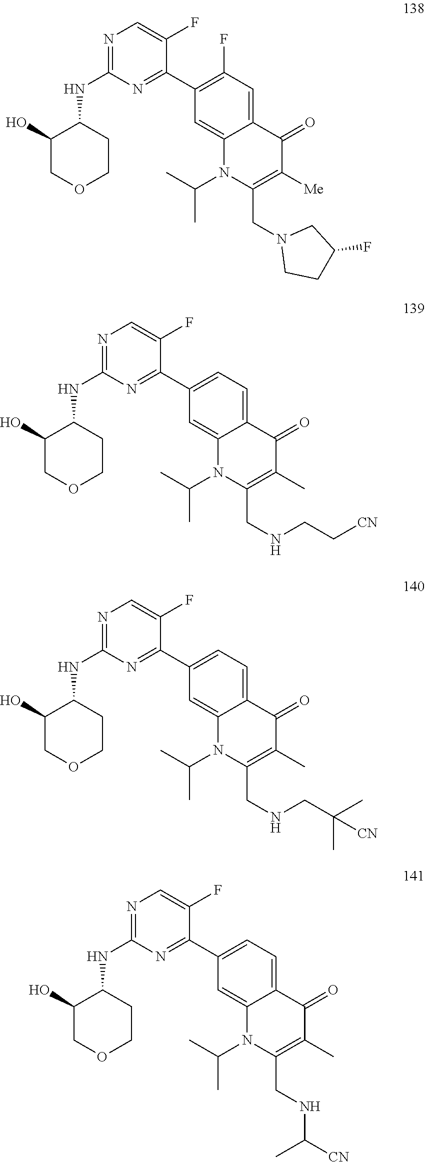 [SMILES: Cc1c(CN2CC[C@@H](F)C2)n(C(C)C)c2cc(-c3nc(N[C@@H]4CCOC[C@H]4O)ncc3F)c(F)cc2c1=O ; Cc1c(CNCCC#N)n(C(C)C)c2cc(-c3nc(N[C@@H]4CCOC[C@H]4O)ncc3F)ccc2c1=O ; Cc1c(CNCC(C)(C)C#N)n(C(C)C)c2cc(-c3nc(N[C@@H]4CCOC[C@H]4O)ncc3F)ccc2c1=O ; Cc1c(CNC(C)C#N)n(C(C)C)c2cc(-c3nc(N[C@@H]4CCOC[C@H]4O)ncc3F)ccc2c1=O]

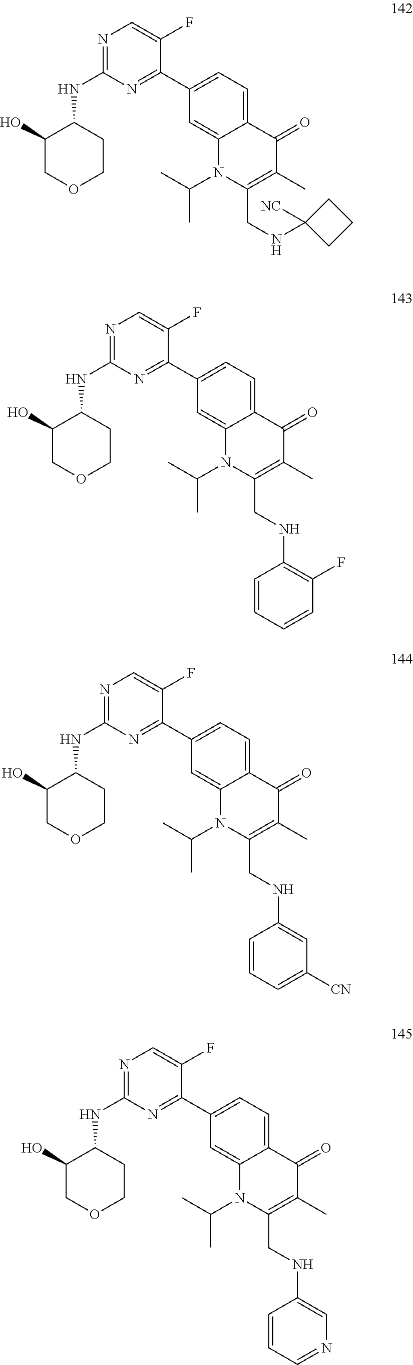 [SMILES: Cc1c(CNC2(C#N)CCC2)n(C(C)C)c2cc(-c3nc(N[C@@H]4CCOC[C@H]4O)ncc3F)ccc2c1=O ; Cc1c(CNc2ccccc2F)n(C(C)C)c2cc(-c3nc(N[C@@H]4CCOC[C@H]4O)ncc3F)ccc2c1=O ; Cc1c(CNc2cccc(C#N)c2)n(C(C)C)c2cc(-c3nc(N[C@@H]4CCOC[C@H]4O)ncc3F)ccc2c1=O ; Cc1c(CNc2cccnc2)n(C(C)C)c2cc(-c3nc(N[C@@H]4CCOC[C@H]4O)ncc3F)ccc2c1=O]

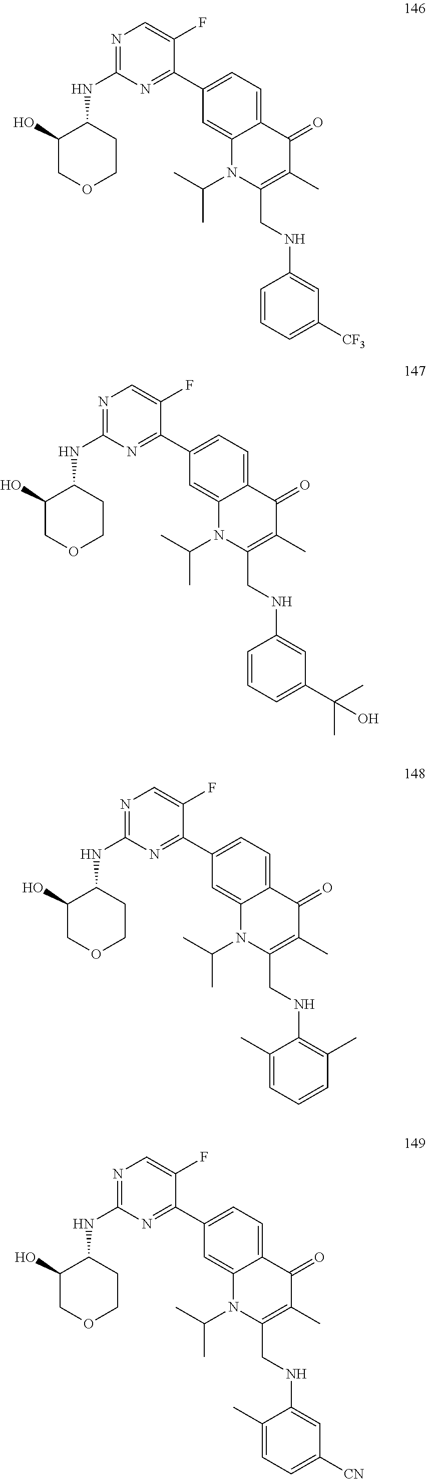 [SMILES: Cc1c(CNc2cccc(C(F)(F)F)c2)n(C(C)C)c2cc(-c3nc(N[C@@H]4CCOC[C@H]4O)ncc3F)ccc2c1=O ; Cc1c(CNc2cccc(C(C)(C)O)c2)n(C(C)C)c2cc(-c3nc(N[C@@H]4CCOC[C@H]4O)ncc3F)ccc2c1=O ; Cc1cccc(C)c1NCc1c(C)c(=O)c2ccc(-c3nc(N[C@@H]4CCOC[C@H]4O)ncc3F)cc2n1C(C)C ; Cc1ccc(C#N)cc1NCc1c(C)c(=O)c2ccc(-c3nc(N[C@@H]4CCOC[C@H]4O)ncc3F)cc2n1C(C)C]

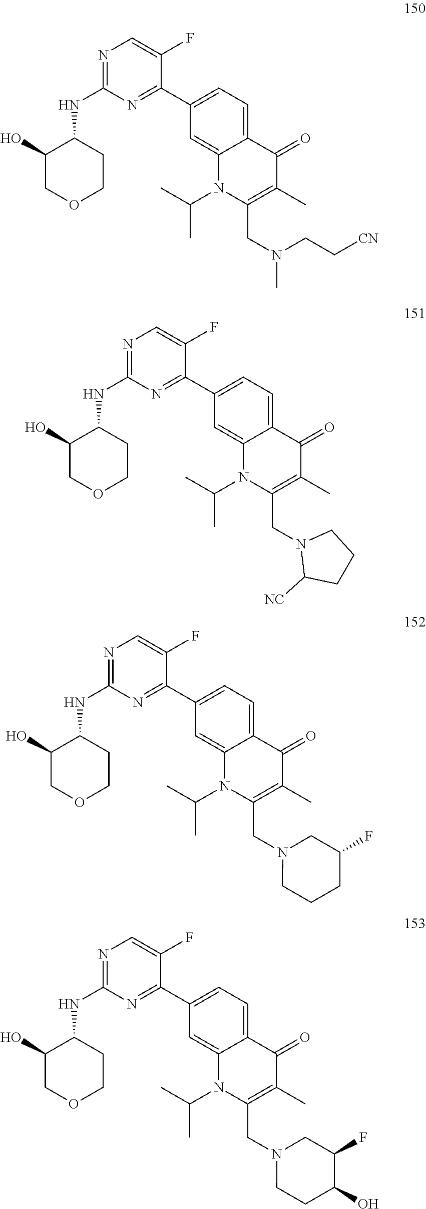 [SMILES: Cc1c(CN(C)CCC#N)n(C(C)C)c2cc(-c3nc(N[C@@H]4CCOC[C@H]4O)ncc3F)ccc2c1=O ; Cc1c(CN2CCCC2C#N)n(C(C)C)c2cc(-c3nc(N[C@@H]4CCOC[C@H]4O)ncc3F)ccc2c1=O ; Cc1c(CN2CCC[C@@H](F)C2)n(C(C)C)c2cc(-c3nc(N[C@@H]4CCOC[C@H]4O)ncc3F)ccc2c1=O ; Cc1c(CN2CC[C@H](O)[C@H](F)C2)n(C(C)C)c2cc(-c3nc(N[C@@H]4CCOC[C@H]4O)ncc3F)ccc2c1=O]

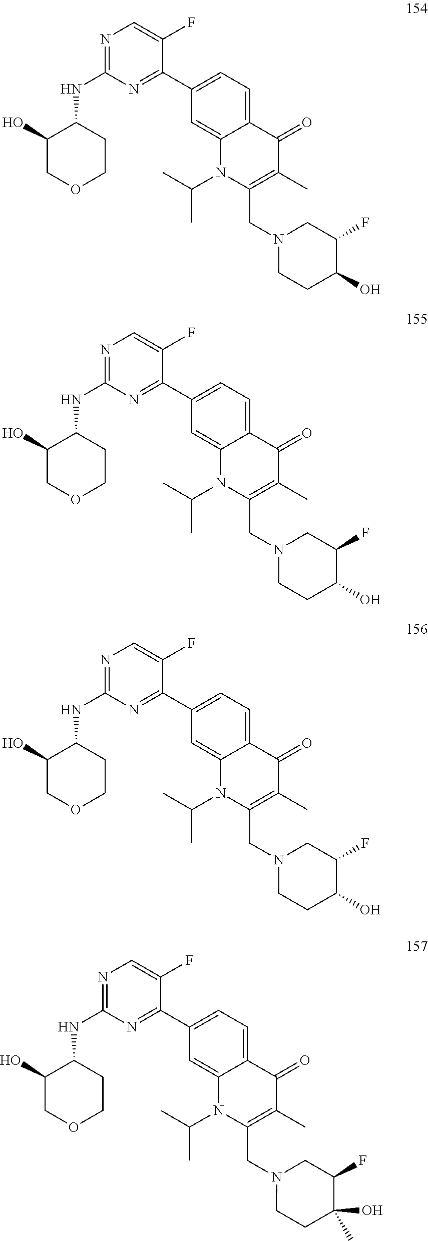 [SMILES: Cc1c(CN2CC[C@H](O)[C@@H](F)C2)n(C(C)C)c2cc(-c3nc(N[C@@H]4CCOC[C@H]4O)ncc3F)ccc2c1=O ; Cc1c(CN2CC[C@@H](O)[C@H](F)C2)n(C(C)C)c2cc(-c3nc(N[C@@H]4CCOC[C@H]4O)ncc3F)ccc2c1=O ; Cc1c(CN2CC[C@@H](O)[C@@H](F)C2)n(C(C)C)c2cc(-c3nc(N[C@@H]4CCOC[C@H]4O)ncc3F)ccc2c1=O ; Cc1c(CN2CC[C@](C)(O)[C@H](F)C2)n(C(C)C)c2cc(-c3nc(N[C@@H]4CCOC[C@H]4O)ncc3F)ccc2c1=O]

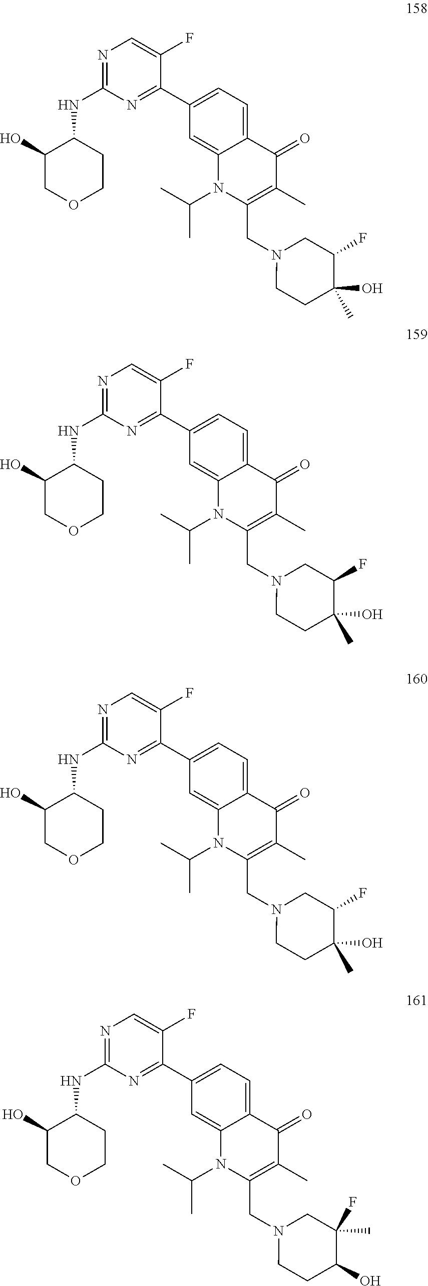 [SMILES: Cc1c(CN2CC[C@](C)(O)[C@@H](F)C2)n(C(C)C)c2cc(-c3nc(N[C@@H]4CCOC[C@H]4O)ncc3F)ccc2c1=O ; Cc1c(CN2CC[C@@](C)(O)[C@H](F)C2)n(C(C)C)c2cc(-c3nc(N[C@@H]4CCOC[C@H]4O)ncc3F)ccc2c1=O ; Cc1c(CN2CC[C@@](C)(O)[C@@H](F)C2)n(C(C)C)c2cc(-c3nc(N[C@@H]4CCOC[C@H]4O)ncc3F)ccc2c1=O ; Cc1c(CN2CC[C@H](O)[C@](C)(F)C2)n(C(C)C)c2cc(-c3nc(N[C@@H]4CCOC[C@H]4O)ncc3F)ccc2c1=O]

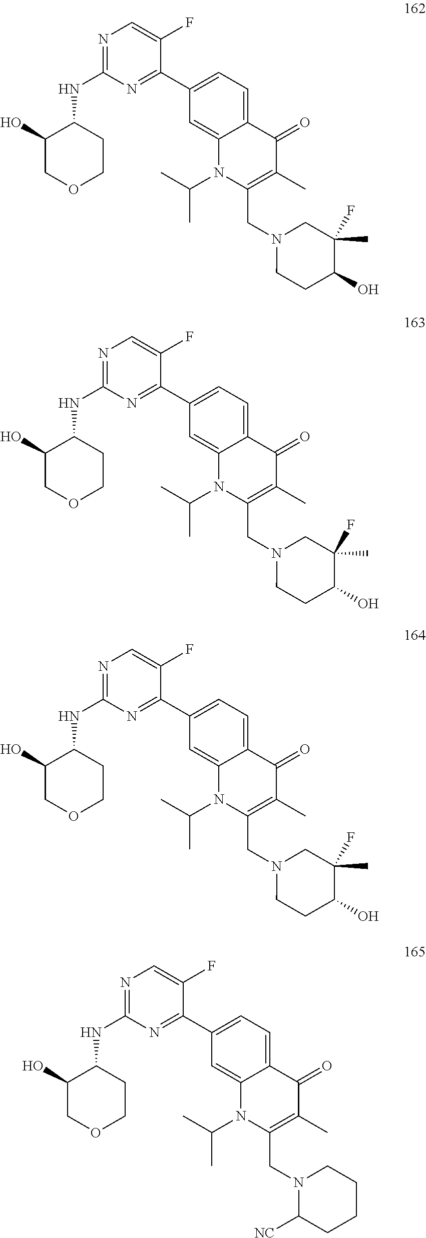 [SMILES: Cc1c(CN2CC[C@H](O)[C@@](C)(F)C2)n(C(C)C)c2cc(-c3nc(N[C@@H]4CCOC[C@H]4O)ncc3F)ccc2c1=O ; Cc1c(CN2CC[C@@H](O)[C@](C)(F)C2)n(C(C)C)c2cc(-c3nc(N[C@@H]4CCOC[C@H]4O)ncc3F)ccc2c1=O ; Cc1c(CN2CC[C@@H](O)[C@@](C)(F)C2)n(C(C)C)c2cc(-c3nc(N[C@@H]4CCOC[C@H]4O)ncc3F)ccc2c1=O ; Cc1c(CN2CCCCC2C#N)n(C(C)C)c2cc(-c3nc(N[C@@H]4CCOC[C@H]4O)ncc3F)ccc2c1=O]

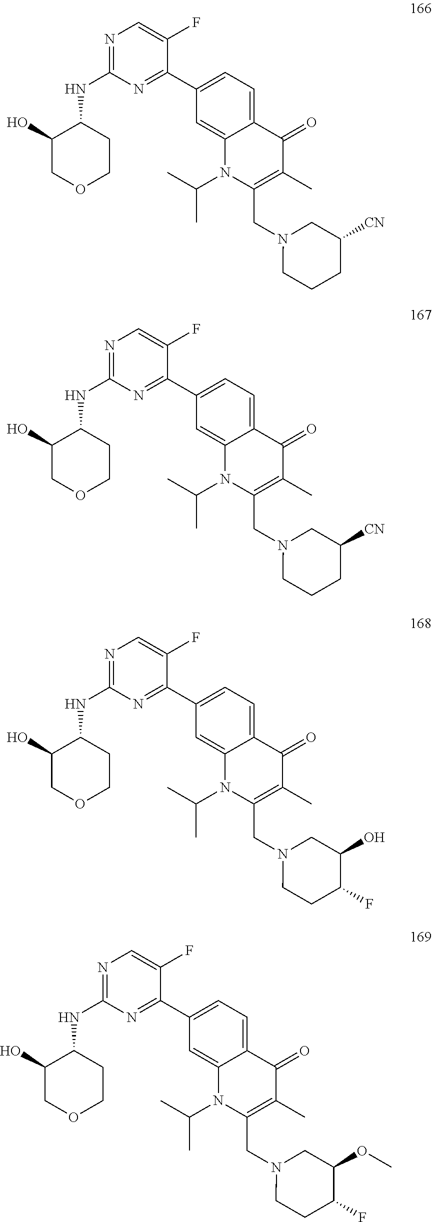 [SMILES: Cc1c(CN2CCC[C@@H](C#N)C2)n(C(C)C)c2cc(-c3nc(N[C@@H]4CCOC[C@H]4O)ncc3F)ccc2c1=O ; Cc1c(CN2CCC[C@H](C#N)C2)n(C(C)C)c2cc(-c3nc(N[C@@H]4CCOC[C@H]4O)ncc3F)ccc2c1=O ; Cc1c(CN2CC[C@@H](F)[C@H](O)C2)n(C(C)C)c2cc(-c3nc(N[C@@H]4CCOC[C@H]4O)ncc3F)ccc2c1=O ; CO[C@@H]1CN(Cc2c(C)c(=O)c3ccc(-c4nc(N[C@@H]5CCOC[C@H]5O)ncc4F)cc3n2C(C)C)CC[C@H]1F]

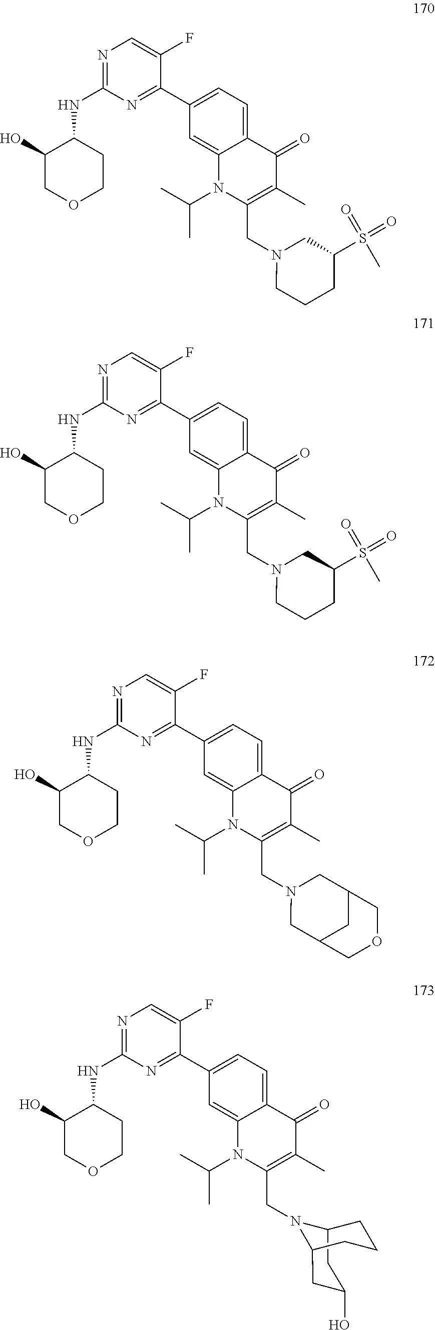 [SMILES: Cc1c(CN2CCC[C@@H](S(C)(=O)=O)C2)n(C(C)C)c2cc(-c3nc(N[C@@H]4CCOC[C@H]4O)ncc3F)ccc2c1=O ; Cc1c(CN2CCC[C@H](S(C)(=O)=O)C2)n(C(C)C)c2cc(-c3nc(N[C@@H]4CCOC[C@H]4O)ncc3F)ccc2c1=O ; Cc1c(CN2CC3COCC(C3)C2)n(C(C)C)c2cc(-c3nc(N[C@@H]4CCOC[C@H]4O)ncc3F)ccc2c1=O ; Cc1c(CN2C3CCCC2CC(O)C3)n(C(C)C)c2cc(-c3nc(N[C@@H]4CCOC[C@H]4O)ncc3F)ccc2c1=O]

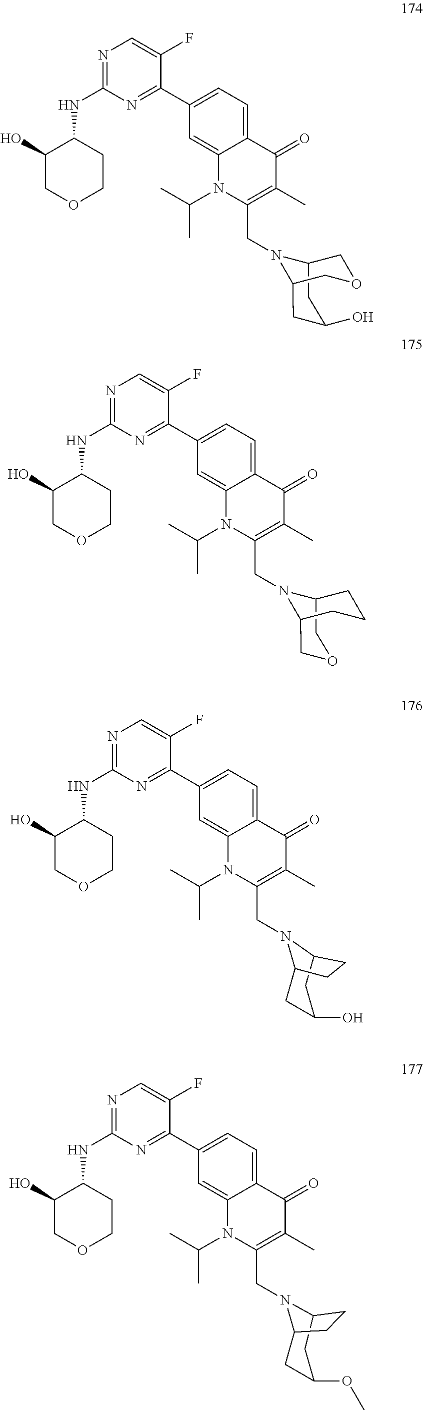 [SMILES: Cc1c(CN2C3COCC2CC(O)C3)n(C(C)C)c2cc(-c3nc(N[C@@H]4CCOC[C@H]4O)ncc3F)ccc2c1=O ; Cc1c(CN2C3CCCC2COC3)n(C(C)C)c2cc(-c3nc(N[C@@H]4CCOC[C@H]4O)ncc3F)ccc2c1=O ; Cc1c(CN2C3CCC2CC(O)C3)n(C(C)C)c2cc(-c3nc(N[C@@H]4CCOC[C@H]4O)ncc3F)ccc2c1=O ; COC1CC2CCC(C1)N2Cc1c(C)c(=O)c2ccc(-c3nc(N[C@@H]4CCOC[C@H]4O)ncc3F)cc2n1C(C)C]

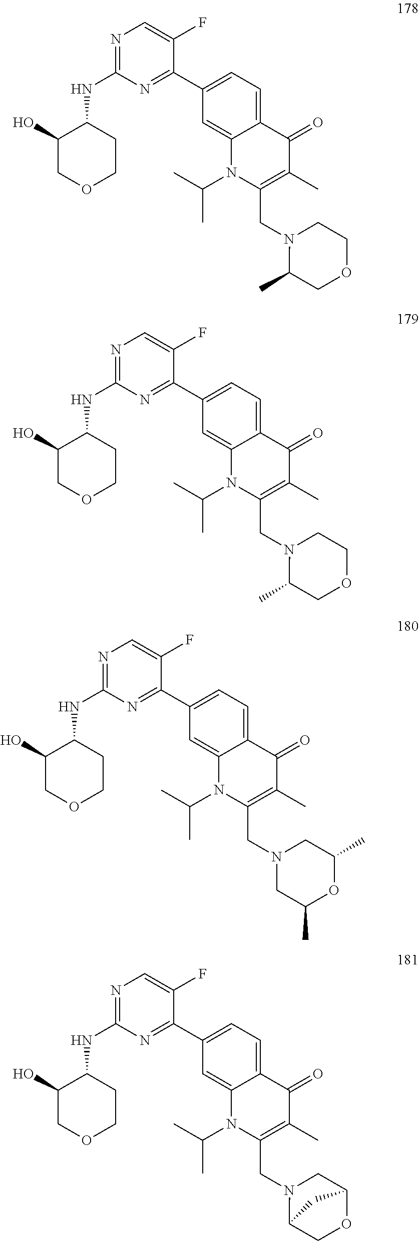 [SMILES: Cc1c(CN2CCOC[C@H]2C)n(C(C)C)c2cc(-c3nc(N[C@@H]4CCOC[C@H]4O)ncc3F)ccc2c1=O ; Cc1c(CN2CCOC[C@@H]2C)n(C(C)C)c2cc(-c3nc(N[C@@H]4CCOC[C@H]4O)ncc3F)ccc2c1=O ; Cc1c(CN2C[C@H](C)O[C@@H](C)C2)n(C(C)C)c2cc(-c3nc(N[C@@H]4CCOC[C@H]4O)ncc3F)ccc2c1=O ; Cc1c(CN2C[C@@H]3C[C@H]2CO3)n(C(C)C)c2cc(-c3nc(N[C@@H]4CCOC[C@H]4O)ncc3F)ccc2c1=O]

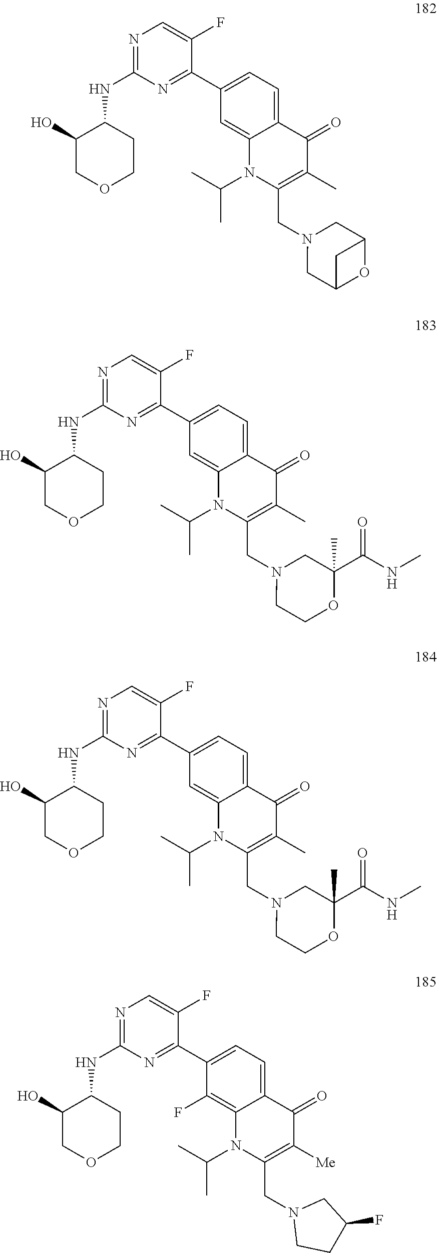 [SMILES: Cc1c(CN2CC3CC(C2)O3)n(C(C)C)c2cc(-c3nc(N[C@@H]4CCOC[C@H]4O)ncc3F)ccc2c1=O ; CNC(=O)[C@]1(C)CN(Cc2c(C)c(=O)c3ccc(-c4nc(N[C@@H]5CCOC[C@H]5O)ncc4F)cc3n2C(C)C)CCO1 ; CNC(=O)[C@@]1(C)CN(Cc2c(C)c(=O)c3ccc(-c4nc(N[C@@H]5CCOC[C@H]5O)ncc4F)cc3n2C(C)C)CCO1 ; Cc1c(CN2CC[C@H](F)C2)n(C(C)C)c2c(F)c(-c3nc(N[C@@H]4CCOC[C@H]4O)ncc3F)ccc2c1=O]

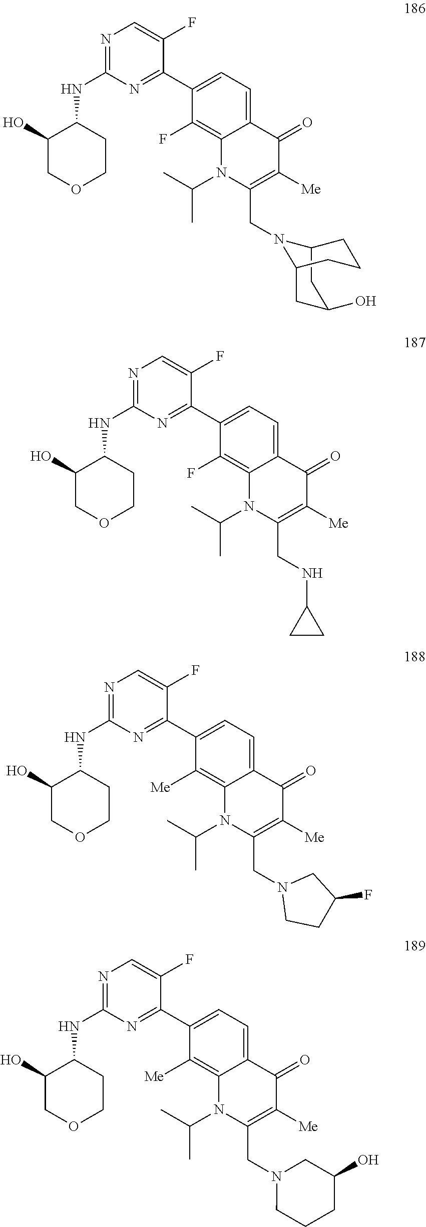 [SMILES: Cc1c(CN2C3CCCC2CC(O)C3)n(C(C)C)c2c(F)c(-c3nc(N[C@@H]4CCOC[C@H]4O)ncc3F)ccc2c1=O ; Cc1c(CNC2CC2)n(C(C)C)c2c(F)c(-c3nc(N[C@@H]4CCOC[C@H]4O)ncc3F)ccc2c1=O ; Cc1c(CN2CC[C@H](F)C2)n(C(C)C)c2c(C)c(-c3nc(N[C@@H]4CCOC[C@H]4O)ncc3F)ccc2c1=O ; Cc1c(CN2CCC[C@H](O)C2)n(C(C)C)c2c(C)c(-c3nc(N[C@@H]4CCOC[C@H]4O)ncc3F)ccc2c1=O]

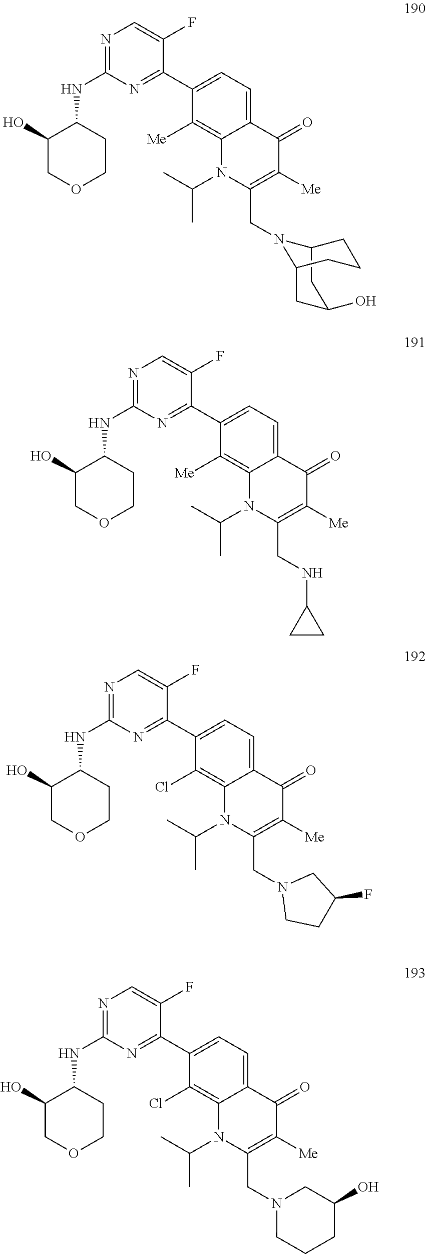 [SMILES: Cc1c(CN2C3CCCC2CC(O)C3)n(C(C)C)c2c(C)c(-c3nc(N[C@@H]4CCOC[C@H]4O)ncc3F)ccc2c1=O ; Cc1c(CNC2CC2)n(C(C)C)c2c(C)c(-c3nc(N[C@@H]4CCOC[C@H]4O)ncc3F)ccc2c1=O ; Cc1c(CN2CC[C@H](F)C2)n(C(C)C)c2c(Cl)c(-c3nc(N[C@@H]4CCOC[C@H]4O)ncc3F)ccc2c1=O ; Cc1c(CN2CCC[C@H](O)C2)n(C(C)C)c2c(Cl)c(-c3nc(N[C@@H]4CCOC[C@H]4O)ncc3F)ccc2c1=O]

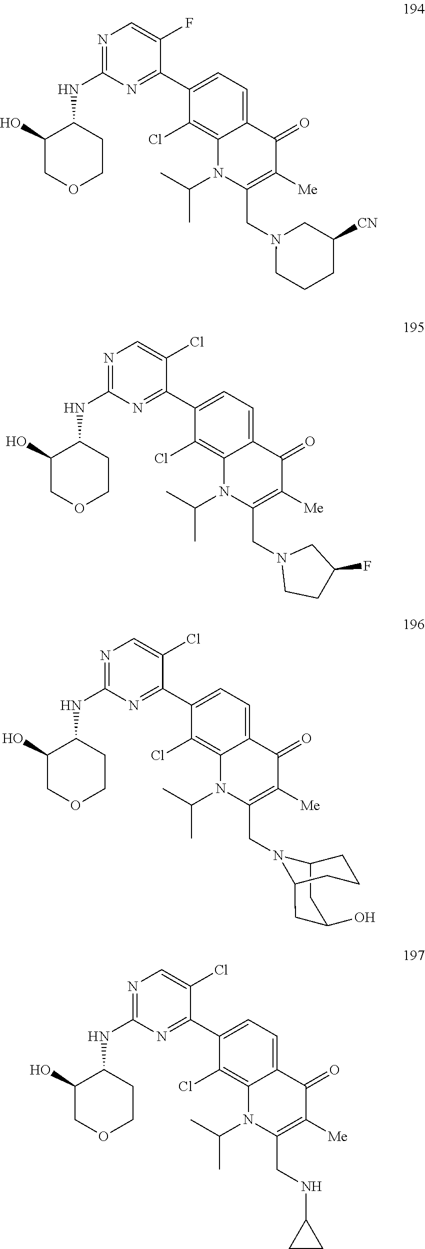 [SMILES: Cc1c(CN2CCC[C@H](C#N)C2)n(C(C)C)c2c(Cl)c(-c3nc(N[C@@H]4CCOC[C@H]4O)ncc3F)ccc2c1=O ; Cc1c(CN2CC[C@H](F)C2)n(C(C)C)c2c(Cl)c(-c3nc(N[C@@H]4CCOC[C@H]4O)ncc3Cl)ccc2c1=O ; Cc1c(CN2C3CCCC2CC(O)C3)n(C(C)C)c2c(Cl)c(-c3nc(N[C@@H]4CCOC[C@H]4O)ncc3Cl)ccc2c1=O ; Cc1c(CNC2CC2)n(C(C)C)c2c(Cl)c(-c3nc(N[C@@H]4CCOC[C@H]4O)ncc3Cl)ccc2c1=O]

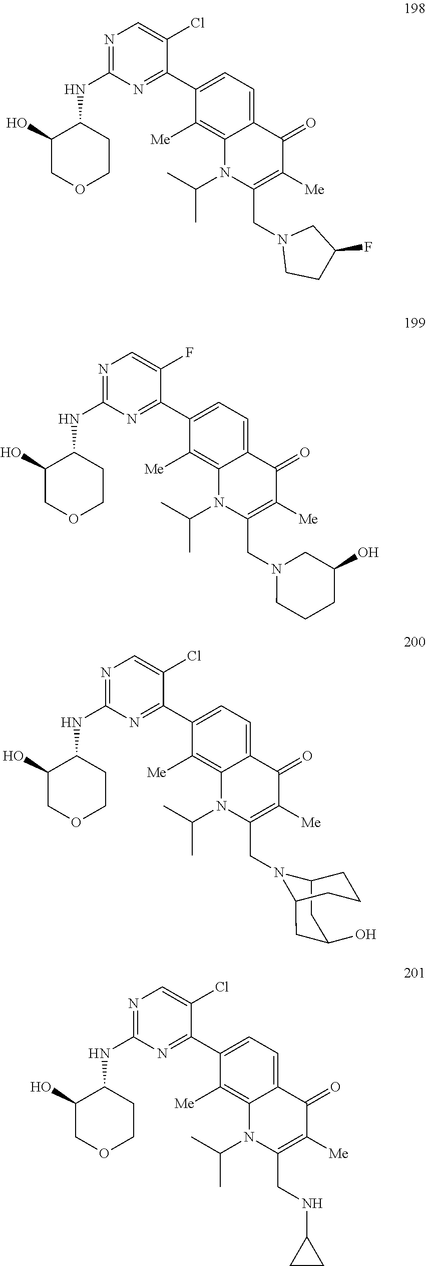 [SMILES: Cc1c(CN2CC[C@H](F)C2)n(C(C)C)c2c(C)c(-c3nc(N[C@@H]4CCOC[C@H]4O)ncc3Cl)ccc2c1=O ; Cc1c(CN2CCC[C@H](O)C2)n(C(C)C)c2c(C)c(-c3nc(N[C@@H]4CCOC[C@H]4O)ncc3F)ccc2c1=O ; Cc1c(CN2C3CCCC2CC(O)C3)n(C(C)C)c2c(C)c(-c3nc(N[C@@H]4CCOC[C@H]4O)ncc3Cl)ccc2c1=O ; Cc1c(CNC2CC2)n(C(C)C)c2c(C)c(-c3nc(N[C@@H]4CCOC[C@H]4O)ncc3Cl)ccc2c1=O]

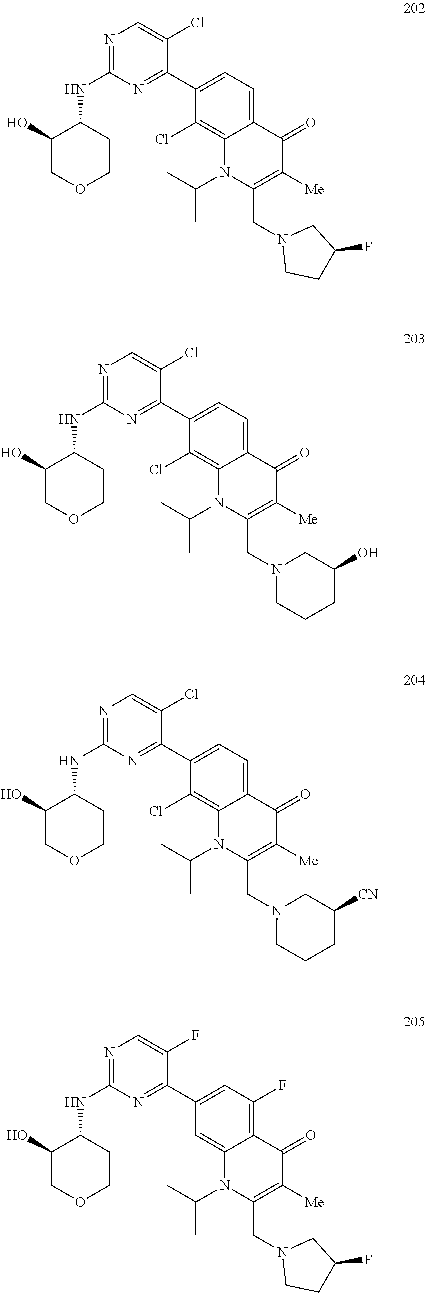 [SMILES: Cc1c(CN2CC[C@H](F)C2)n(C(C)C)c2c(Cl)c(-c3nc(N[C@@H]4CCOC[C@H]4O)ncc3Cl)ccc2c1=O ; Cc1c(CN2CCC[C@H](O)C2)n(C(C)C)c2c(Cl)c(-c3nc(N[C@@H]4CCOC[C@H]4O)ncc3Cl)ccc2c1=O ; Cc1c(CN2CCC[C@H](C#N)C2)n(C(C)C)c2c(Cl)c(-c3nc(N[C@@H]4CCOC[C@H]4O)ncc3Cl)ccc2c1=O ; Cc1c(CN2CC[C@H](F)C2)n(C(C)C)c2cc(-c3nc(N[C@@H]4CCOC[C@H]4O)ncc3F)cc(F)c2c1=O]

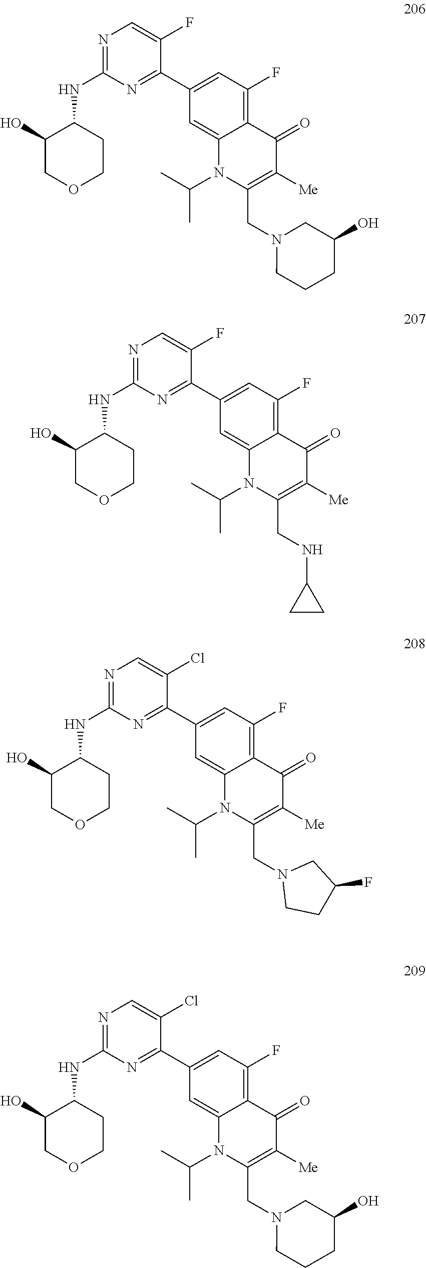 [SMILES: Cc1c(CN2CCC[C@H](O)C2)n(C(C)C)c2cc(-c3nc(N[C@@H]4CCOC[C@H]4O)ncc3F)cc(F)c2c1=O ; Cc1c(CNC2CC2)n(C(C)C)c2cc(-c3nc(N[C@@H]4CCOC[C@H]4O)ncc3F)cc(F)c2c1=O ; Cc1c(CN2CC[C@H](F)C2)n(C(C)C)c2cc(-c3nc(N[C@@H]4CCOC[C@H]4O)ncc3Cl)cc(F)c2c1=O ; Cc1c(CN2CCC[C@H](O)C2)n(C(C)C)c2cc(-c3nc(N[C@@H]4CCOC[C@H]4O)ncc3Cl)cc(F)c2c1=O]

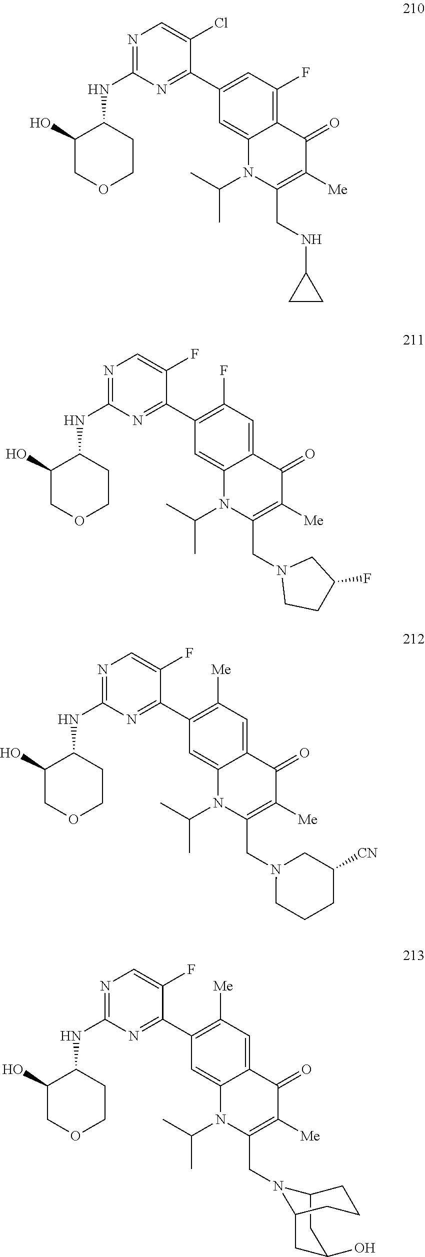 [SMILES: Cc1c(CNC2CC2)n(C(C)C)c2cc(-c3nc(N[C@@H]4CCOC[C@H]4O)ncc3Cl)cc(F)c2c1=O ; Cc1c(CN2CC[C@@H](F)C2)n(C(C)C)c2cc(-c3nc(N[C@@H]4CCOC[C@H]4O)ncc3F)c(F)cc2c1=O ; Cc1cc2c(=O)c(C)c(CN3CCC[C@@H](C#N)C3)n(C(C)C)c2cc1-c1nc(N[C@@H]2CCOC[C@H]2O)ncc1F ; Cc1cc2c(=O)c(C)c(CN3C4CCCC3CC(O)C4)n(C(C)C)c2cc1-c1nc(N[C@@H]2CCOC[C@H]2O)ncc1F]

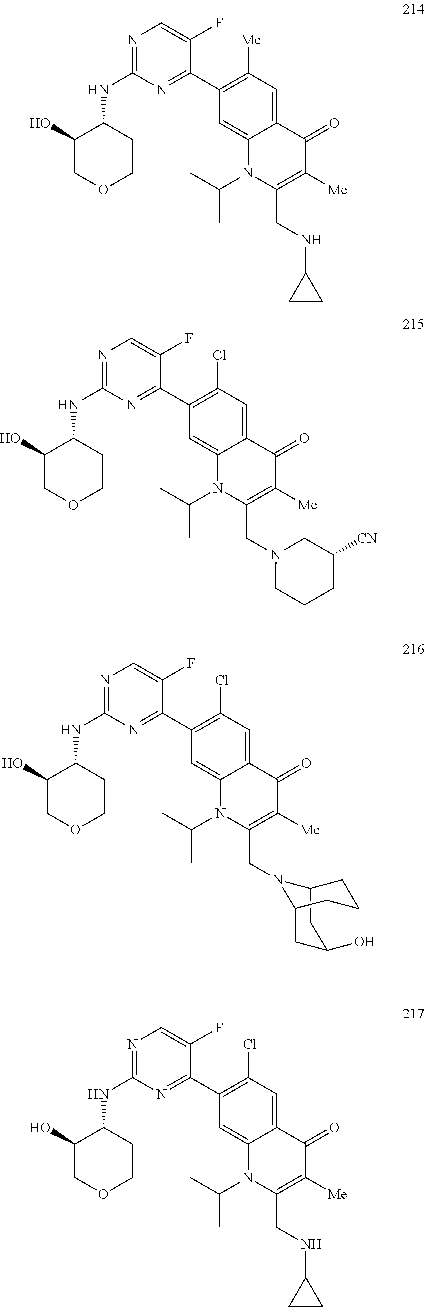 [SMILES: Cc1cc2c(=O)c(C)c(CNC3CC3)n(C(C)C)c2cc1-c1nc(N[C@@H]2CCOC[C@H]2O)ncc1F ; Cc1c(CN2CCC[C@@H](C#N)C2)n(C(C)C)c2cc(-c3nc(N[C@@H]4CCOC[C@H]4O)ncc3F)c(Cl)cc2c1=O ; Cc1c(CN2C3CCCC2CC(O)C3)n(C(C)C)c2cc(-c3nc(N[C@@H]4CCOC[C@H]4O)ncc3F)c(Cl)cc2c1=O ; Cc1c(CNC2CC2)n(C(C)C)c2cc(-c3nc(N[C@@H]4CCOC[C@H]4O)ncc3F)c(Cl)cc2c1=O]

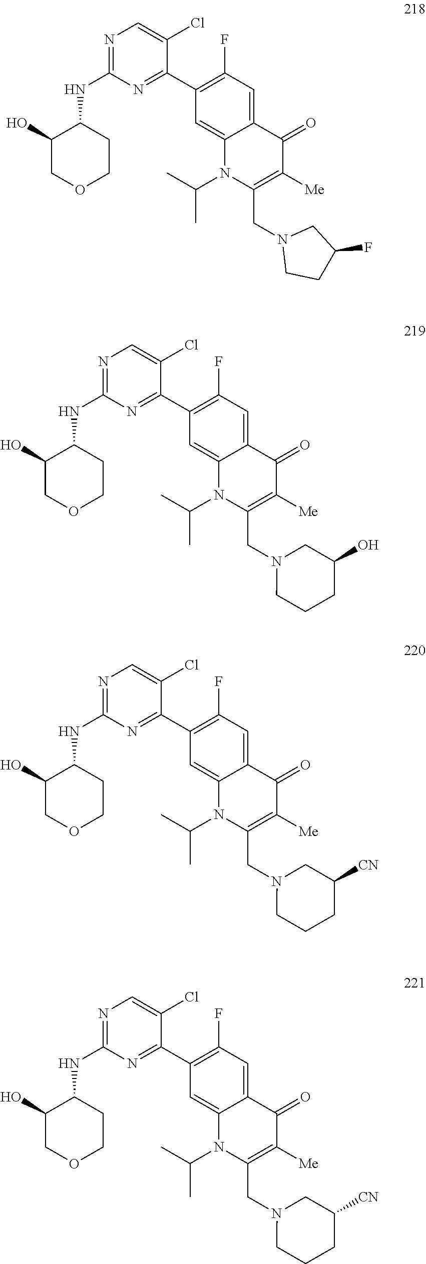 [SMILES: Cc1c(CN2CC[C@H](F)C2)n(C(C)C)c2cc(-c3nc(N[C@@H]4CCOC[C@H]4O)ncc3Cl)c(F)cc2c1=O ; Cc1c(CN2CCC[C@H](O)C2)n(C(C)C)c2cc(-c3nc(N[C@@H]4CCOC[C@H]4O)ncc3Cl)c(F)cc2c1=O ; Cc1c(CN2CCC[C@H](C#N)C2)n(C(C)C)c2cc(-c3nc(N[C@@H]4CCOC[C@H]4O)ncc3Cl)c(F)cc2c1=O ; Cc1c(CN2CCC[C@@H](C#N)C2)n(C(C)C)c2cc(-c3nc(N[C@@H]4CCOC[C@H]4O)ncc3Cl)c(F)cc2c1=O]

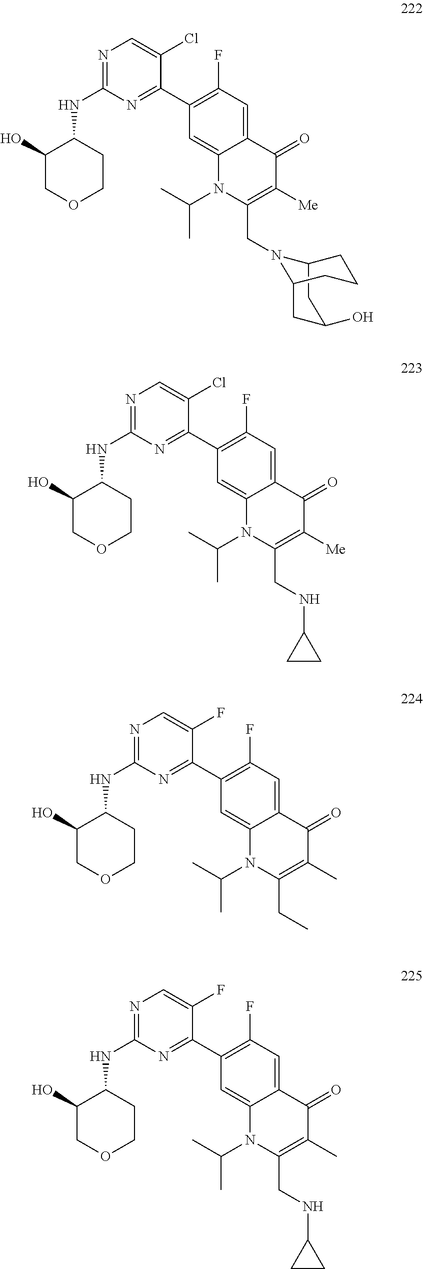 [SMILES: Cc1c(CN2C3CCCC2CC(O)C3)n(C(C)C)c2cc(-c3nc(N[C@@H]4CCOC[C@H]4O)ncc3Cl)c(F)cc2c1=O ; Cc1c(CNC2CC2)n(C(C)C)c2cc(-c3nc(N[C@@H]4CCOC[C@H]4O)ncc3Cl)c(F)cc2c1=O ; CCc1c(C)c(=O)c2cc(F)c(-c3nc(N[C@@H]4CCOC[C@H]4O)ncc3F)cc2n1C(C)C ; Cc1c(CNC2CC2)n(C(C)C)c2cc(-c3nc(N[C@@H]4CCOC[C@H]4O)ncc3F)c(F)cc2c1=O]

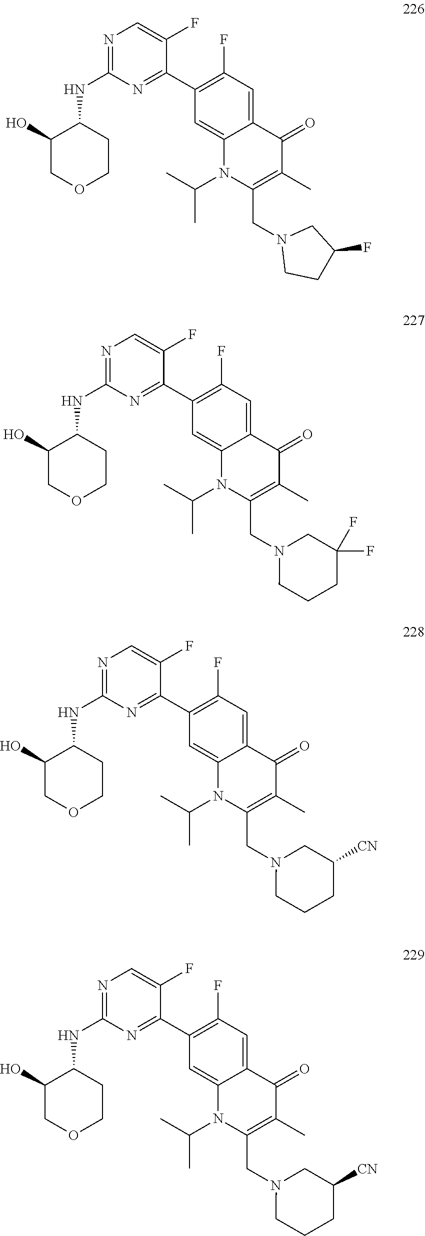 [SMILES: Cc1c(CN2CC[C@H](F)C2)n(C(C)C)c2cc(-c3nc(N[C@@H]4CCOC[C@H]4O)ncc3F)c(F)cc2c1=O ; Cc1c(CN2CCCC(F)(F)C2)n(C(C)C)c2cc(-c3nc(N[C@@H]4CCOC[C@H]4O)ncc3F)c(F)cc2c1=O ; Cc1c(CN2CCC[C@@H](C#N)C2)n(C(C)C)c2cc(-c3nc(N[C@@H]4CCOC[C@H]4O)ncc3F)c(F)cc2c1=O ; Cc1c(CN2CCC[C@H](C#N)C2)n(C(C)C)c2cc(-c3nc(N[C@@H]4CCOC[C@H]4O)ncc3F)c(F)cc2c1=O]

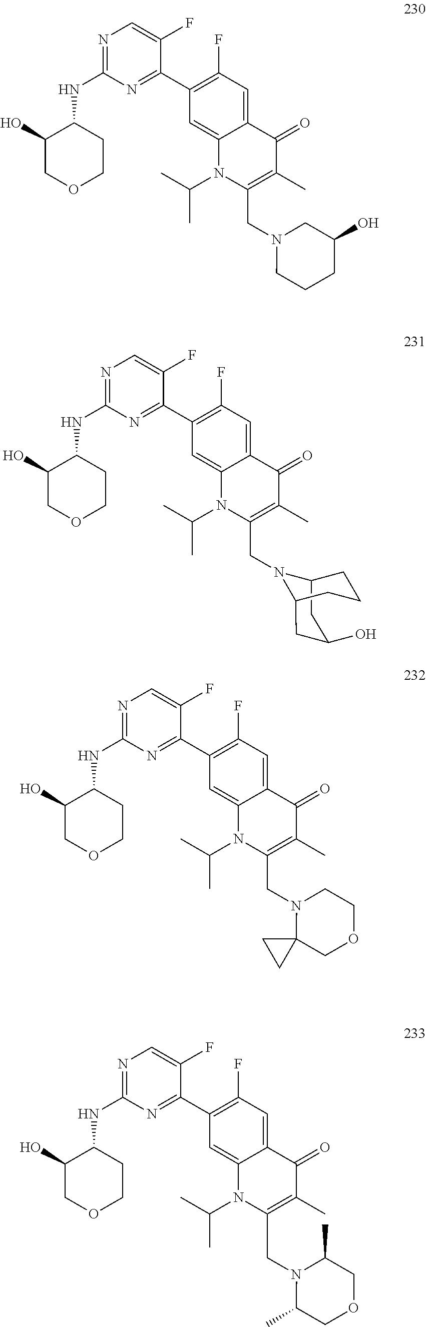 [SMILES: Cc1c(CN2CCC[C@H](O)C2)n(C(C)C)c2cc(-c3nc(N[C@@H]4CCOC[C@H]4O)ncc3F)c(F)cc2c1=O ; Cc1c(CN2C3CCCC2CC(O)C3)n(C(C)C)c2cc(-c3nc(N[C@@H]4CCOC[C@H]4O)ncc3F)c(F)cc2c1=O ; Cc1c(CN2CCOCC23CC3)n(C(C)C)c2cc(-c3nc(N[C@@H]4CCOC[C@H]4O)ncc3F)c(F)cc2c1=O ; Cc1c(CN2[C@@H](C)COC[C@@H]2C)n(C(C)C)c2cc(-c3nc(N[C@@H]4CCOC[C@H]4O)ncc3F)c(F)cc2c1=O]

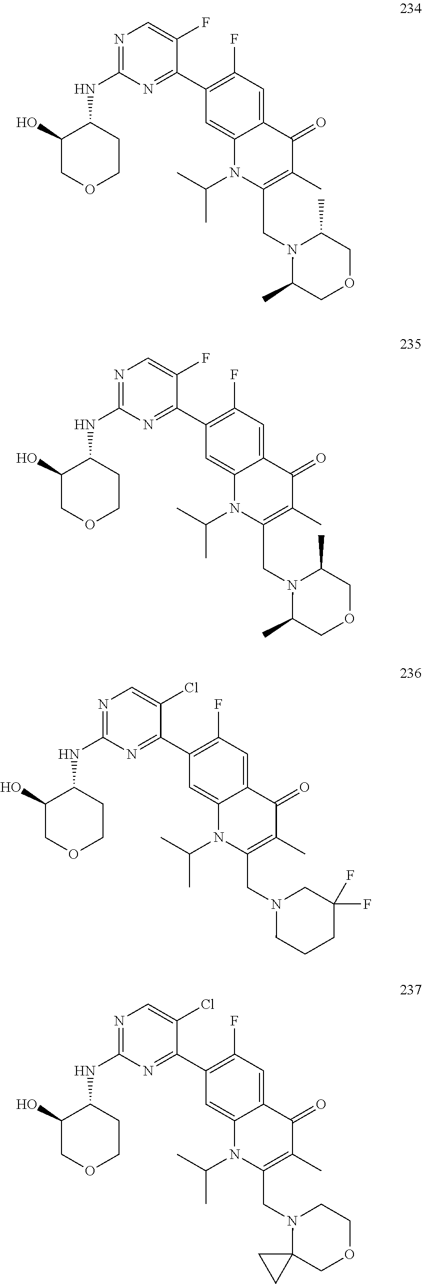 [SMILES: Cc1c(CN2[C@H](C)COC[C@H]2C)n(C(C)C)c2cc(-c3nc(N[C@@H]4CCOC[C@H]4O)ncc3F)c(F)cc2c1=O ; Cc1c(CN2[C@H](C)COC[C@@H]2C)n(C(C)C)c2cc(-c3nc(N[C@@H]4CCOC[C@H]4O)ncc3F)c(F)cc2c1=O ; Cc1c(CN2CCCC(F)(F)C2)n(C(C)C)c2cc(-c3nc(N[C@@H]4CCOC[C@H]4O)ncc3Cl)c(F)cc2c1=O ; Cc1c(CN2CCOCC23CC3)n(C(C)C)c2cc(-c3nc(N[C@@H]4CCOC[C@H]4O)ncc3Cl)c(F)cc2c1=O]

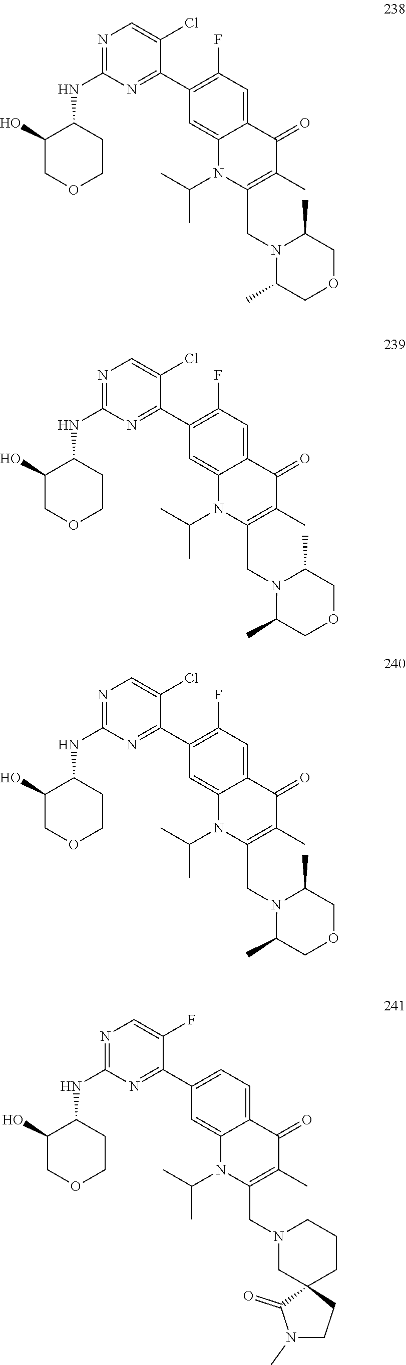 [SMILES: Cc1c(CN2[C@@H](C)COC[C@@H]2C)n(C(C)C)c2cc(-c3nc(N[C@@H]4CCOC[C@H]4O)ncc3Cl)c(F)cc2c1=O ; Cc1c(CN2[C@H](C)COC[C@H]2C)n(C(C)C)c2cc(-c3nc(N[C@@H]4CCOC[C@H]4O)ncc3Cl)c(F)cc2c1=O ; Cc1c(CN2[C@H](C)COC[C@@H]2C)n(C(C)C)c2cc(-c3nc(N[C@@H]4CCOC[C@H]4O)ncc3Cl)c(F)cc2c1=O ; Cc1c(CN2CCC[C@@]3(CCN(C)C3=O)C2)n(C(C)C)c2cc(-c3nc(N[C@@H]4CCOC[C@H]4O)ncc3F)ccc2c1=O]

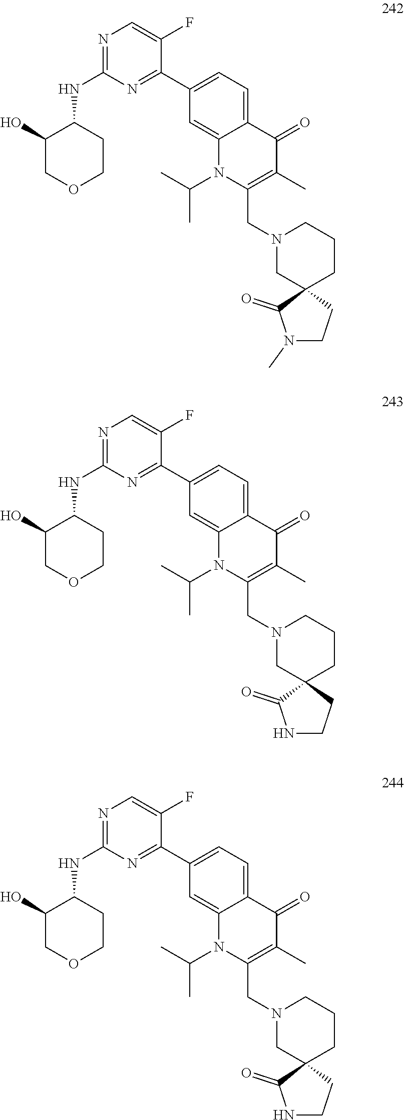 [SMILES: Cc1c(CN2CCC[C@]3(CCN(C)C3=O)C2)n(C(C)C)c2cc(-c3nc(N[C@@H]4CCOC[C@H]4O)ncc3F)ccc2c1=O ; Cc1c(CN2CCC[C@@]3(CCNC3=O)C2)n(C(C)C)c2cc(-c3nc(N[C@@H]4CCOC[C@H]4O)ncc3F)ccc2c1=O ; Cc1c(CN2CCC[C@]3(CCNC3=O)C2)n(C(C)C)c2cc(-c3nc(N[C@@H]4CCOC[C@H]4O)ncc3F)ccc2c1=O]

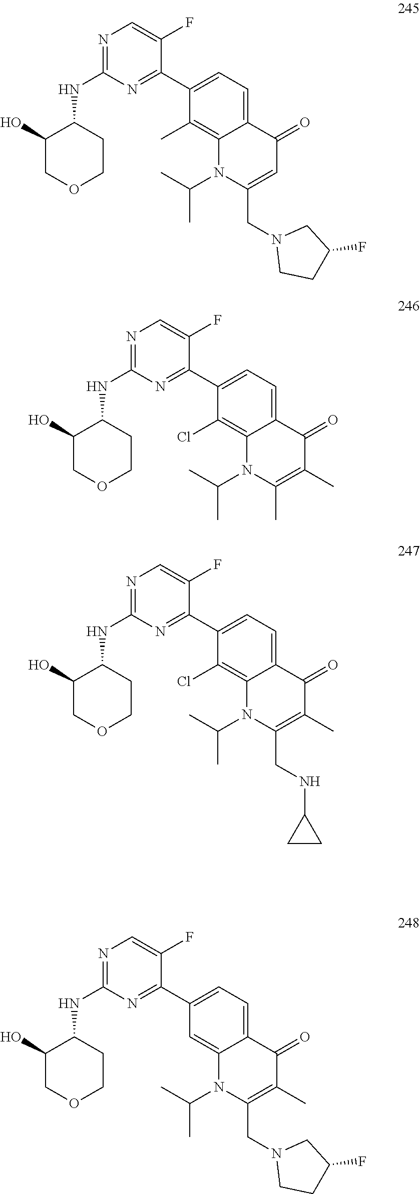

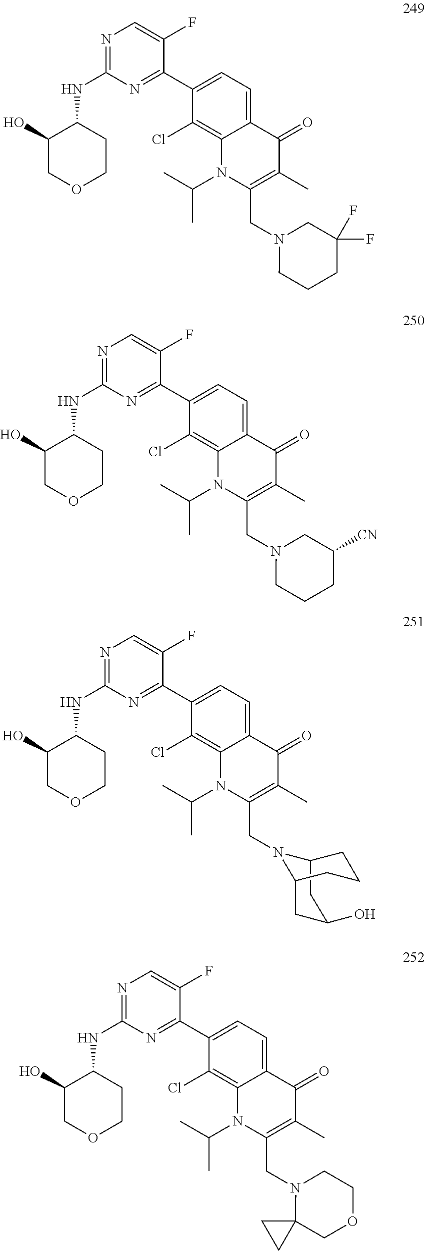 [SMILES: Cc1c(CN2CCCC(F)(F)C2)n(C(C)C)c2c(Cl)c(-c3nc(N[C@@H]4CCOC[C@H]4O)ncc3F)ccc2c1=O ; Cc1c(CN2CCC[C@@H](C#N)C2)n(C(C)C)c2c(Cl)c(-c3nc(N[C@@H]4CCOC[C@H]4O)ncc3F)ccc2c1=O ; Cc1c(CN2C3CCCC2CC(O)C3)n(C(C)C)c2c(Cl)c(-c3nc(N[C@@H]4CCOC[C@H]4O)ncc3F)ccc2c1=O ; Cc1c(CN2CCOCC23CC3)n(C(C)C)c2c(Cl)c(-c3nc(N[C@@H]4CCOC[C@H]4O)ncc3F)ccc2c1=O]

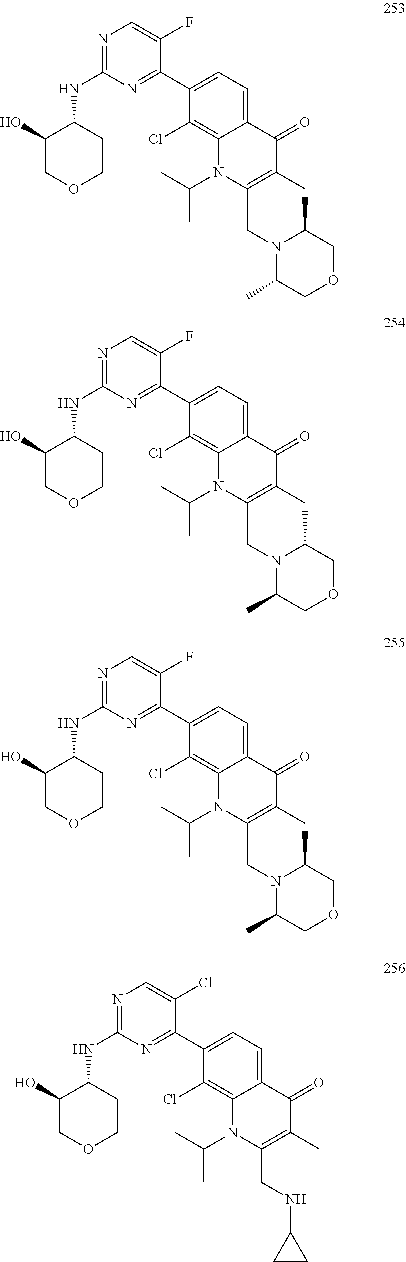 [SMILES: Cc1c(CN2[C@@H](C)COC[C@@H]2C)n(C(C)C)c2c(Cl)c(-c3nc(N[C@@H]4CCOC[C@H]4O)ncc3F)ccc2c1=O ; Cc1c(CN2[C@H](C)COC[C@H]2C)n(C(C)C)c2c(Cl)c(-c3nc(N[C@@H]4CCOC[C@H]4O)ncc3F)ccc2c1=O ; Cc1c(CN2[C@H](C)COC[C@@H]2C)n(C(C)C)c2c(Cl)c(-c3nc(N[C@@H]4CCOC[C@H]4O)ncc3F)ccc2c1=O ; Cc1c(CNC2CC2)n(C(C)C)c2c(Cl)c(-c3nc(N[C@@H]4CCOC[C@H]4O)ncc3Cl)ccc2c1=O]

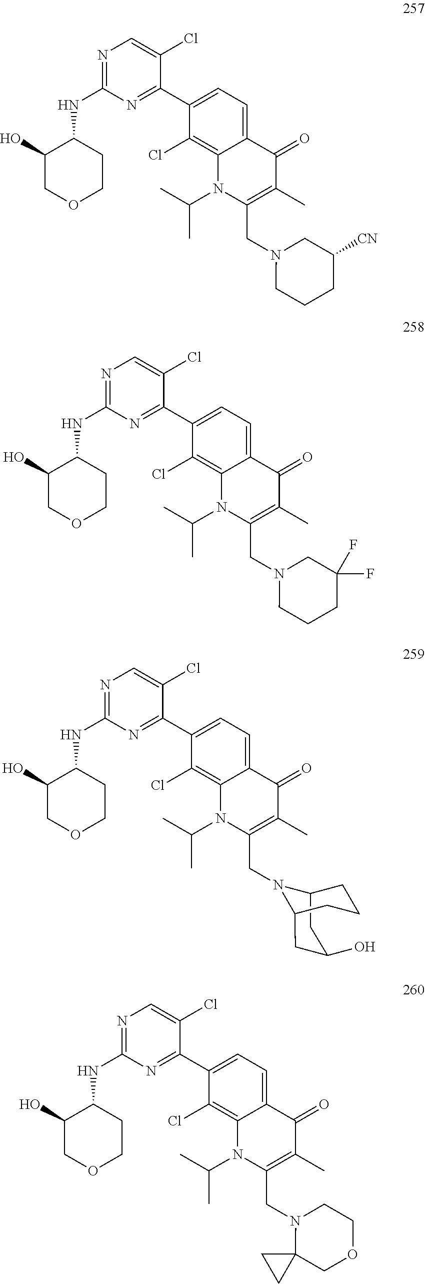 [SMILES: Cc1c(CN2CCC[C@@H](C#N)C2)n(C(C)C)c2c(Cl)c(-c3nc(N[C@@H]4CCOC[C@H]4O)ncc3Cl)ccc2c1=O ; Cc1c(CN2CCCC(F)(F)C2)n(C(C)C)c2c(Cl)c(-c3nc(N[C@@H]4CCOC[C@H]4O)ncc3Cl)ccc2c1=O ; Cc1c(CN2C3CCCC2CC(O)C3)n(C(C)C)c2c(Cl)c(-c3nc(N[C@@H]4CCOC[C@H]4O)ncc3Cl)ccc2c1=O ; Cc1c(CN2CCOCC23CC3)n(C(C)C)c2c(Cl)c(-c3nc(N[C@@H]4CCOC[C@H]4O)ncc3Cl)ccc2c1=O]

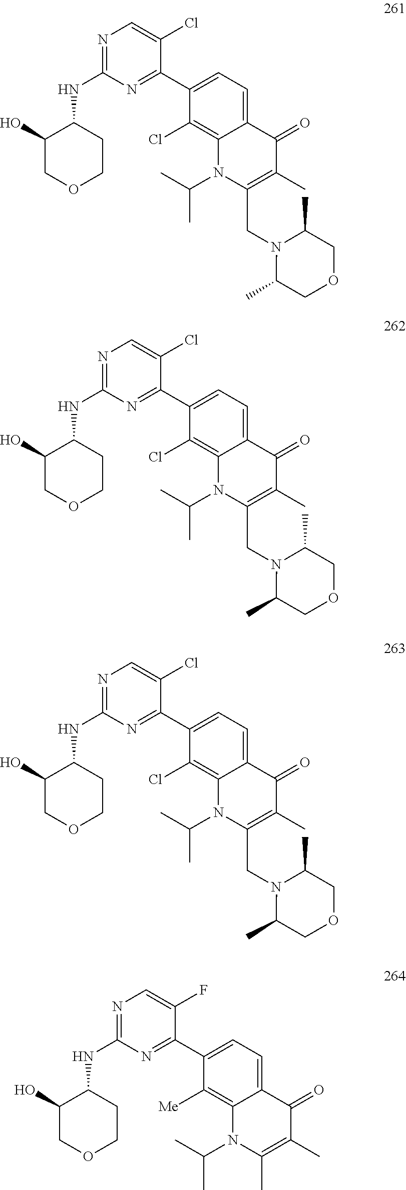 [SMILES: Cc1c(CN2[C@@H](C)COC[C@@H]2C)n(C(C)C)c2c(Cl)c(-c3nc(N[C@@H]4CCOC[C@H]4O)ncc3Cl)ccc2c1=O ; Cc1c(CN2[C@H](C)COC[C@H]2C)n(C(C)C)c2c(Cl)c(-c3nc(N[C@@H]4CCOC[C@H]4O)ncc3Cl)ccc2c1=O ; Cc1c(CN2[C@H](C)COC[C@@H]2C)n(C(C)C)c2c(Cl)c(-c3nc(N[C@@H]4CCOC[C@H]4O)ncc3Cl)ccc2c1=O ; Cc1c(C)n(C(C)C)c2c(C)c(-c3nc(N[C@@H]4CCOC[C@H]4O)ncc3F)ccc2c1=O]

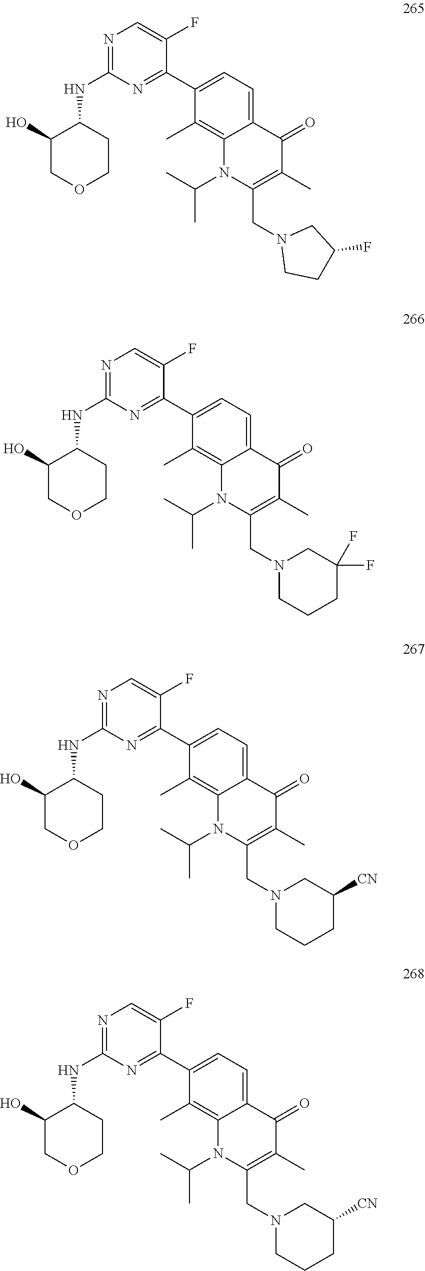 [SMILES: Cc1c(CN2CC[C@@H](F)C2)n(C(C)C)c2c(C)c(-c3nc(N[C@@H]4CCOC[C@H]4O)ncc3F)ccc2c1=O ; Cc1c(CN2CCCC(F)(F)C2)n(C(C)C)c2c(C)c(-c3nc(N[C@@H]4CCOC[C@H]4O)ncc3F)ccc2c1=O ; Cc1c(CN2CCC[C@H](C#N)C2)n(C(C)C)c2c(C)c(-c3nc(N[C@@H]4CCOC[C@H]4O)ncc3F)ccc2c1=O ; Cc1c(CN2CCC[C@@H](C#N)C2)n(C(C)C)c2c(C)c(-c3nc(N[C@@H]4CCOC[C@H]4O)ncc3F)ccc2c1=O]

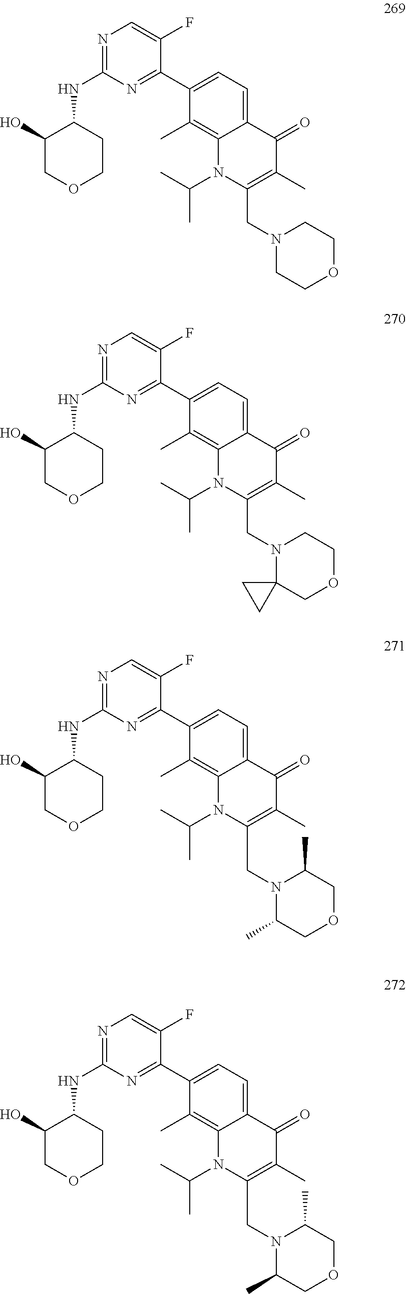 [SMILES: Cc1c(CN2CCOCC2)n(C(C)C)c2c(C)c(-c3nc(N[C@@H]4CCOC[C@H]4O)ncc3F)ccc2c1=O ; Cc1c(CN2CCOCC23CC3)n(C(C)C)c2c(C)c(-c3nc(N[C@@H]4CCOC[C@H]4O)ncc3F)ccc2c1=O ; Cc1c(CN2[C@@H](C)COC[C@@H]2C)n(C(C)C)c2c(C)c(-c3nc(N[C@@H]4CCOC[C@H]4O)ncc3F)ccc2c1=O ; Cc1c(CN2[C@H](C)COC[C@H]2C)n(C(C)C)c2c(C)c(-c3nc(N[C@@H]4CCOC[C@H]4O)ncc3F)ccc2c1=O]

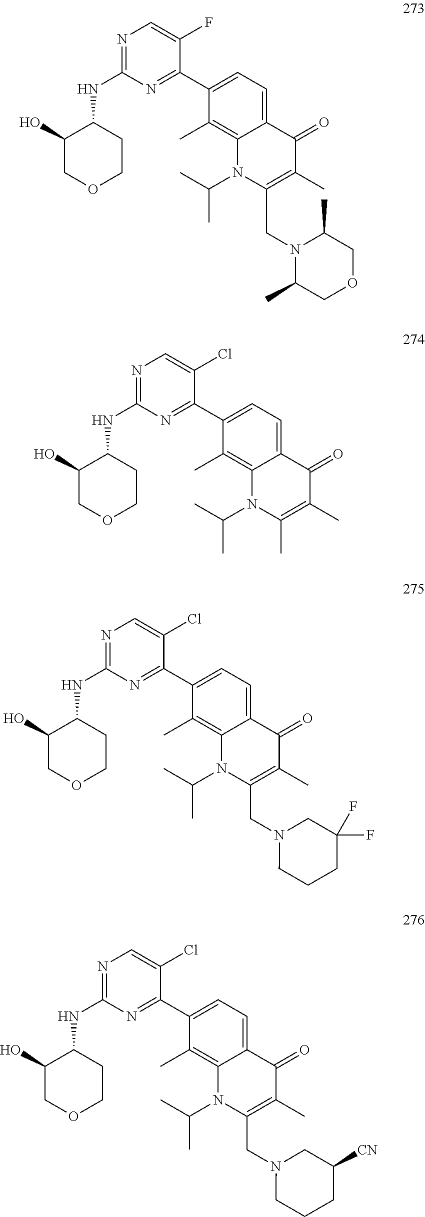 [SMILES: Cc1c(CN2[C@H](C)COC[C@@H]2C)n(C(C)C)c2c(C)c(-c3nc(N[C@@H]4CCOC[C@H]4O)ncc3F)ccc2c1=O ; Cc1c(C)n(C(C)C)c2c(C)c(-c3nc(N[C@@H]4CCOC[C@H]4O)ncc3Cl)ccc2c1=O ; Cc1c(CN2CCCC(F)(F)C2)n(C(C)C)c2c(C)c(-c3nc(N[C@@H]4CCOC[C@H]4O)ncc3Cl)ccc2c1=O ; Cc1c(CN2CCC[C@H](C#N)C2)n(C(C)C)c2c(C)c(-c3nc(N[C@@H]4CCOC[C@H]4O)ncc3Cl)ccc2c1=O]

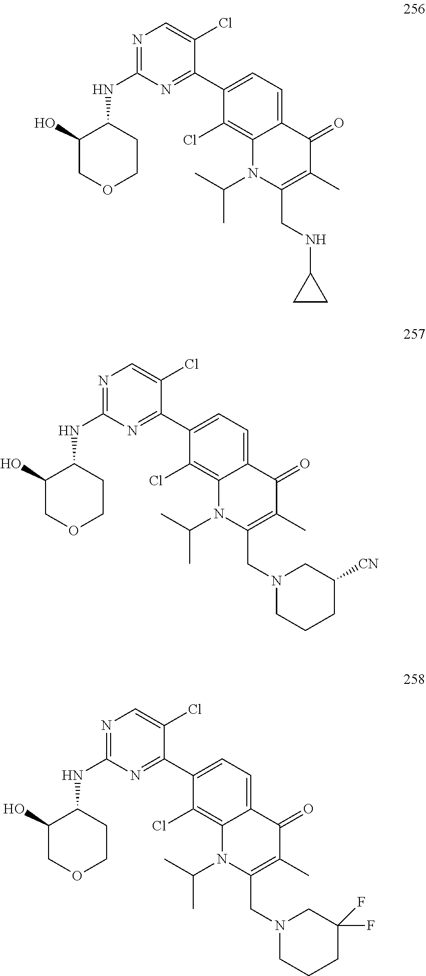 [SMILES: Cc1c(CNC2CC2)n(C(C)C)c2c(Cl)c(-c3nc(N[C@@H]4CCOC[C@H]4O)ncc3Cl)ccc2c1=O ; Cc1c(CN2CCC[C@@H](C#N)C2)n(C(C)C)c2c(Cl)c(-c3nc(N[C@@H]4CCOC[C@H]4O)ncc3Cl)ccc2c1=O ; Cc1c(CN2CCCC(F)(F)C2)n(C(C)C)c2c(Cl)c(-c3nc(N[C@@H]4CCOC[C@H]4O)ncc3Cl)ccc2c1=O]

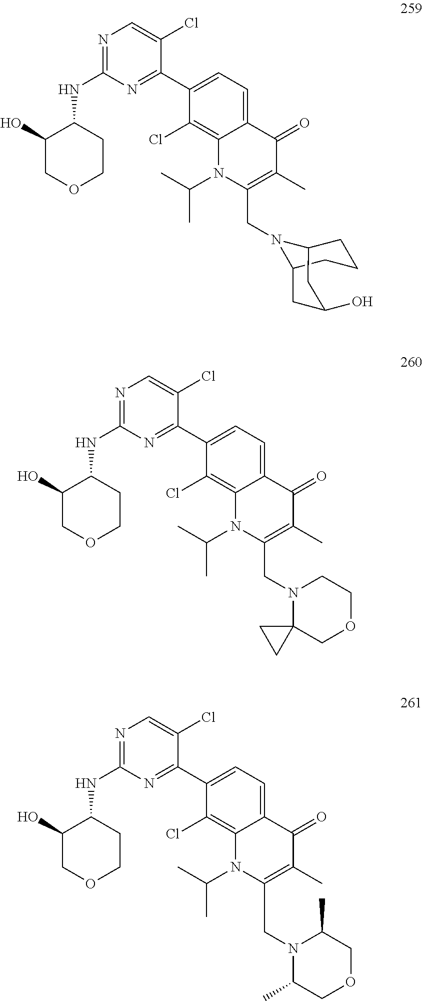 [SMILES: Cc1c(CN2C3CCCC2CC(O)C3)n(C(C)C)c2c(Cl)c(-c3nc(N[C@@H]4CCOC[C@H]4O)ncc3Cl)ccc2c1=O ; Cc1c(CN2CCOCC23CC3)n(C(C)C)c2c(Cl)c(-c3nc(N[C@@H]4CCOC[C@H]4O)ncc3Cl)ccc2c1=O ; Cc1c(CN2[C@@H](C)COC[C@@H]2C)n(C(C)C)c2c(Cl)c(-c3nc(N[C@@H]4CCOC[C@H]4O)ncc3Cl)ccc2c1=O]

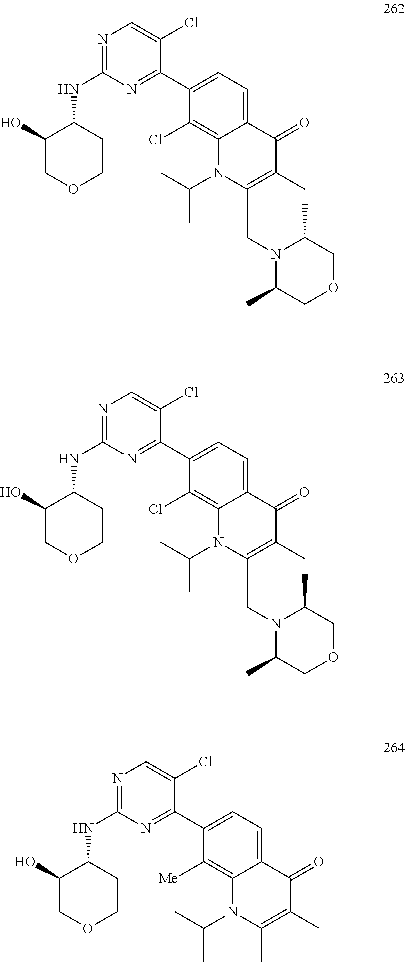 [SMILES: Cc1c(CN2[C@H](C)COC[C@H]2C)n(C(C)C)c2c(Cl)c(-c3nc(N[C@@H]4CCOC[C@H]4O)ncc3Cl)ccc2c1=O ; Cc1c(CN2[C@H](C)COC[C@@H]2C)n(C(C)C)c2c(Cl)c(-c3nc(N[C@@H]4CCOC[C@H]4O)ncc3Cl)ccc2c1=O ; Cc1c(C)n(C(C)C)c2c(C)c(-c3nc(N[C@@H]4CCOC[C@H]4O)ncc3Cl)ccc2c1=O]

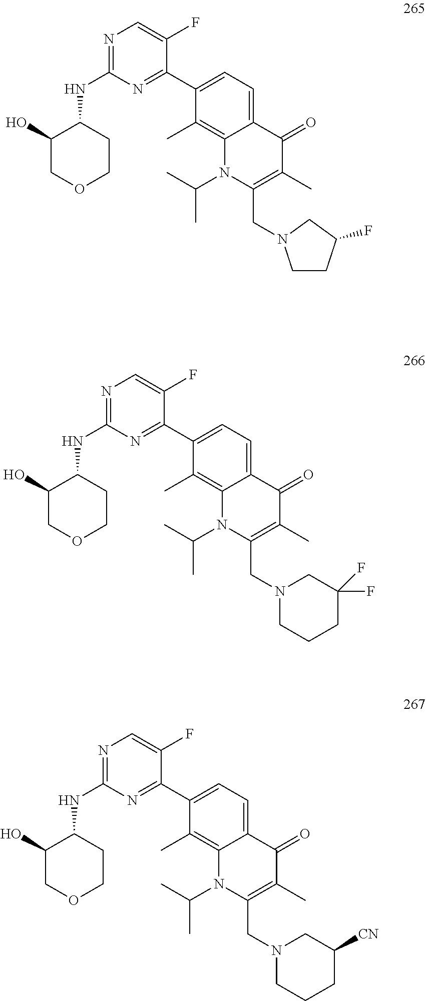 [SMILES: Cc1c(CN2CC[C@@H](F)C2)n(C(C)C)c2c(C)c(-c3nc(N[C@@H]4CCOC[C@H]4O)ncc3F)ccc2c1=O ; Cc1c(CN2CCCC(F)(F)C2)n(C(C)C)c2c(C)c(-c3nc(N[C@@H]4CCOC[C@H]4O)ncc3F)ccc2c1=O ; Cc1c(CN2CCC[C@H](C#N)C2)n(C(C)C)c2c(C)c(-c3nc(N[C@@H]4CCOC[C@H]4O)ncc3F)ccc2c1=O]

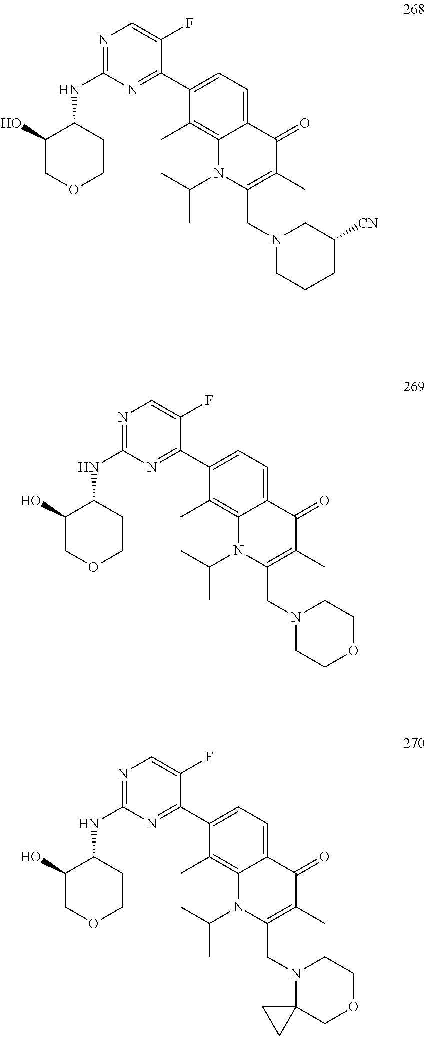 [SMILES: Cc1c(CN2CCC[C@@H](C#N)C2)n(C(C)C)c2c(C)c(-c3nc(N[C@@H]4CCOC[C@H]4O)ncc3F)ccc2c1=O ; Cc1c(CN2CCOCC2)n(C(C)C)c2c(C)c(-c3nc(N[C@@H]4CCOC[C@H]4O)ncc3F)ccc2c1=O ; Cc1c(CN2CCOCC23CC3)n(C(C)C)c2c(C)c(-c3nc(N[C@@H]4CCOC[C@H]4O)ncc3F)ccc2c1=O]

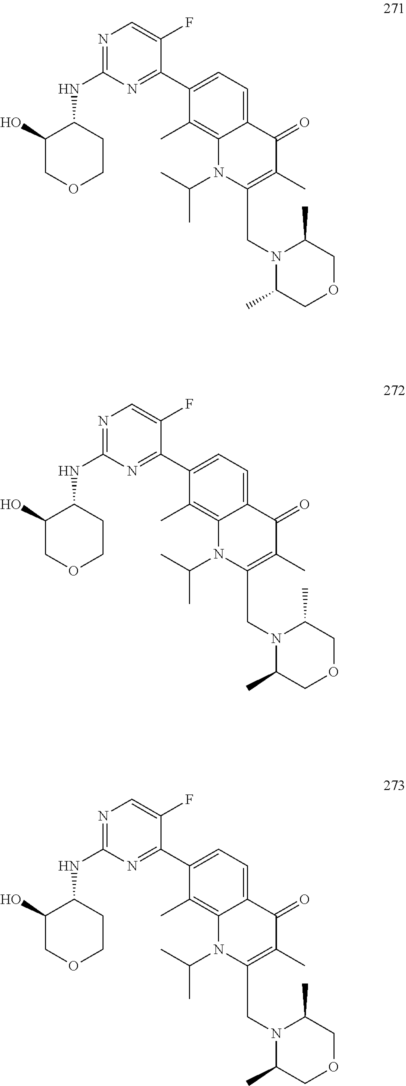 [SMILES: Cc1c(CN2[C@@H](C)COC[C@@H]2C)n(C(C)C)c2c(C)c(-c3nc(N[C@@H]4CCOC[C@H]4O)ncc3F)ccc2c1=O ; Cc1c(CN2[C@H](C)COC[C@H]2C)n(C(C)C)c2c(C)c(-c3nc(N[C@@H]4CCOC[C@H]4O)ncc3F)ccc2c1=O ; Cc1c(CN2[C@H](C)COC[C@@H]2C)n(C(C)C)c2c(C)c(-c3nc(N[C@@H]4CCOC[C@H]4O)ncc3F)ccc2c1=O]

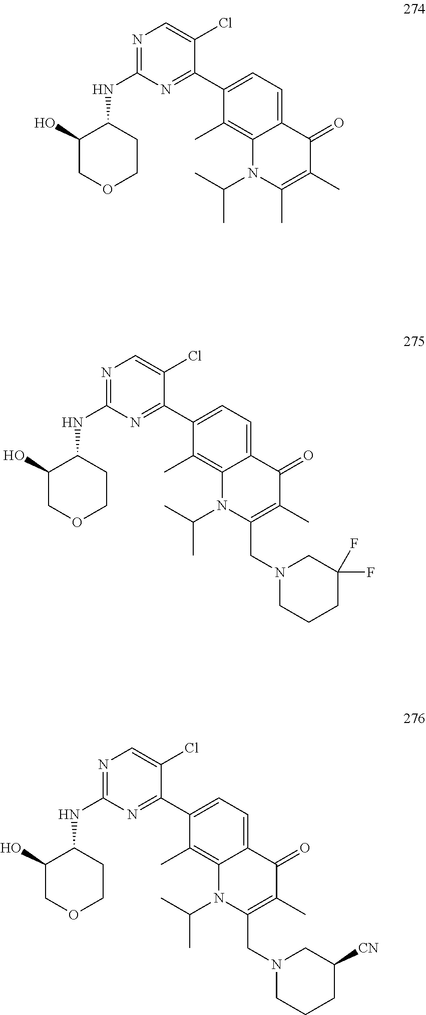 [SMILES: Cc1c(C)n(C(C)C)c2c(C)c(-c3nc(N[C@@H]4CCOC[C@H]4O)ncc3Cl)ccc2c1=O ; Cc1c(CN2CCCC(F)(F)C2)n(C(C)C)c2c(C)c(-c3nc(N[C@@H]4CCOC[C@H]4O)ncc3Cl)ccc2c1=O ; Cc1c(CN2CCC[C@H](C#N)C2)n(C(C)C)c2c(C)c(-c3nc(N[C@@H]4CCOC[C@H]4O)ncc3Cl)ccc2c1=O]

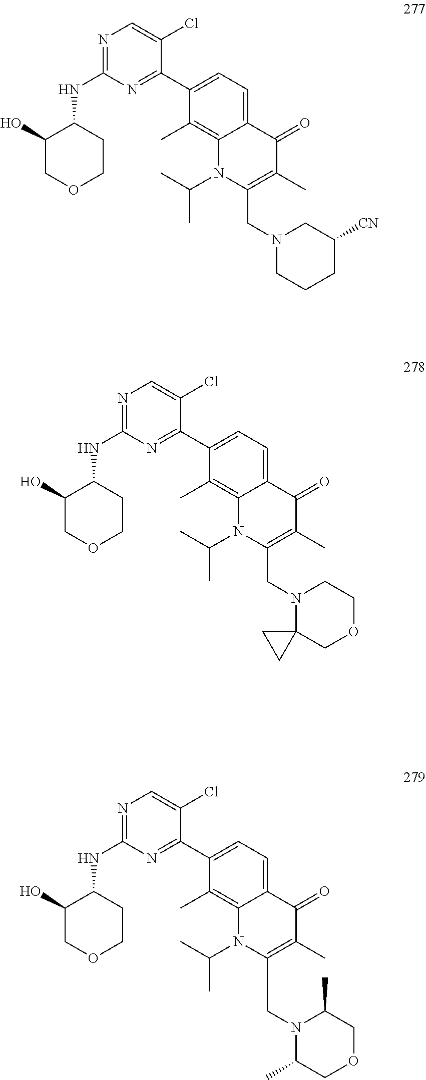 [SMILES: Cc1c(CN2CCC[C@@H](C#N)C2)n(C(C)C)c2c(C)c(-c3nc(N[C@@H]4CCOC[C@H]4O)ncc3Cl)ccc2c1=O ; Cc1c(CN2CCOCC23CC3)n(C(C)C)c2c(C)c(-c3nc(N[C@@H]4CCOC[C@H]4O)ncc3Cl)ccc2c1=O ; Cc1c(CN2[C@@H](C)COC[C@@H]2C)n(C(C)C)c2c(C)c(-c3nc(N[C@@H]4CCOC[C@H]4O)ncc3Cl)ccc2c1=O]

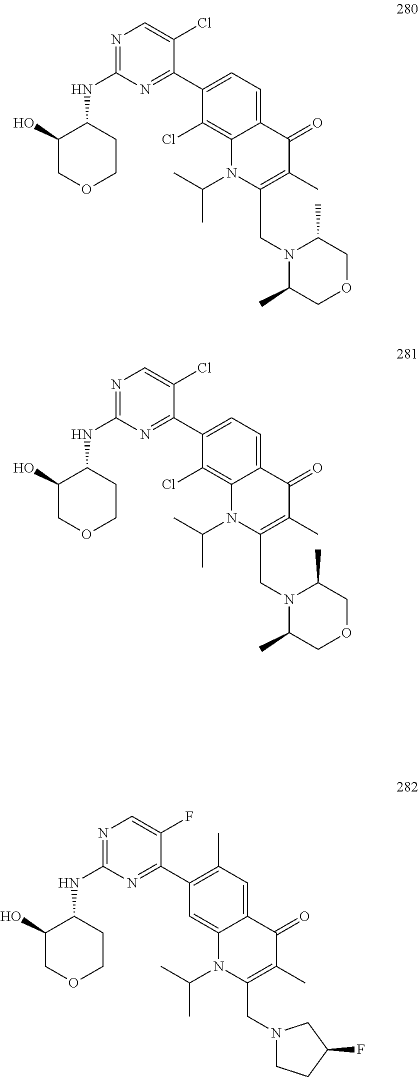 [SMILES: Cc1c(CN2[C@H](C)COC[C@H]2C)n(C(C)C)c2c(Cl)c(-c3nc(N[C@@H]4CCOC[C@H]4O)ncc3Cl)ccc2c1=O ; Cc1c(CN2[C@H](C)COC[C@@H]2C)n(C(C)C)c2c(Cl)c(-c3nc(N[C@@H]4CCOC[C@H]4O)ncc3Cl)ccc2c1=O ; Cc1cc2c(=O)c(C)c(CN3CC[C@H](F)C3)n(C(C)C)c2cc1-c1nc(N[C@@H]2CCOC[C@H]2O)ncc1F]

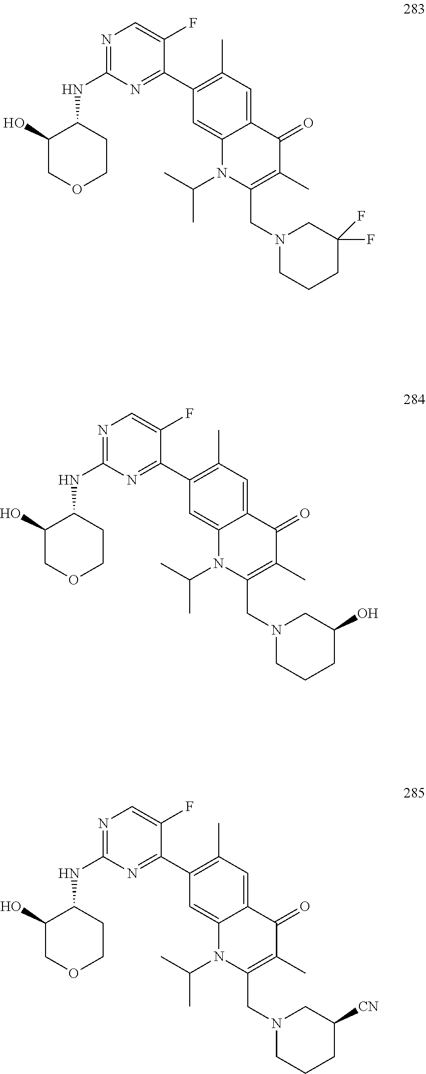 [SMILES: Cc1cc2c(=O)c(C)c(CN3CCCC(F)(F)C3)n(C(C)C)c2cc1-c1nc(N[C@@H]2CCOC[C@H]2O)ncc1F ; Cc1cc2c(=O)c(C)c(CN3CCC[C@H](O)C3)n(C(C)C)c2cc1-c1nc(N[C@@H]2CCOC[C@H]2O)ncc1F ; Cc1cc2c(=O)c(C)c(CN3CCC[C@H](C#N)C3)n(C(C)C)c2cc1-c1nc(N[C@@H]2CCOC[C@H]2O)ncc1F]

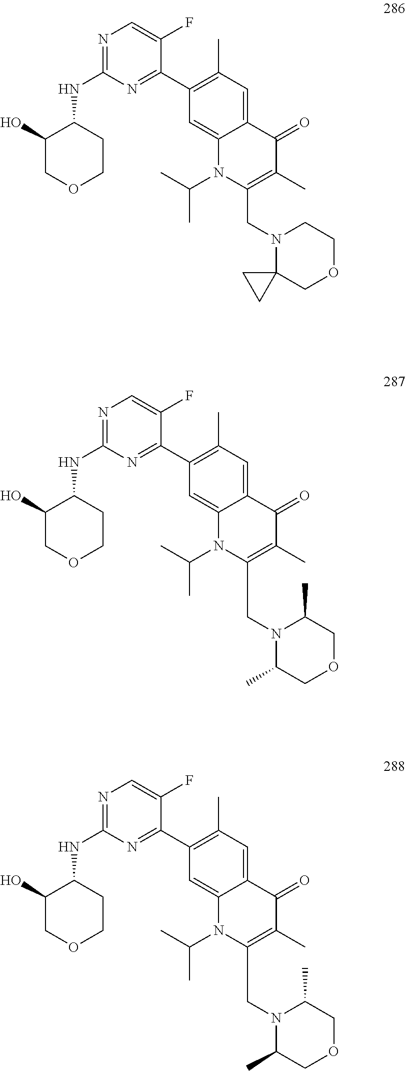 [SMILES: Cc1cc2c(=O)c(C)c(CN3CCOCC34CC4)n(C(C)C)c2cc1-c1nc(N[C@@H]2CCOC[C@H]2O)ncc1F ; Cc1cc2c(=O)c(C)c(CN3[C@@H](C)COC[C@@H]3C)n(C(C)C)c2cc1-c1nc(N[C@@H]2CCOC[C@H]2O)ncc1F ; Cc1cc2c(=O)c(C)c(CN3[C@H](C)COC[C@H]3C)n(C(C)C)c2cc1-c1nc(N[C@@H]2CCOC[C@H]2O)ncc1F]

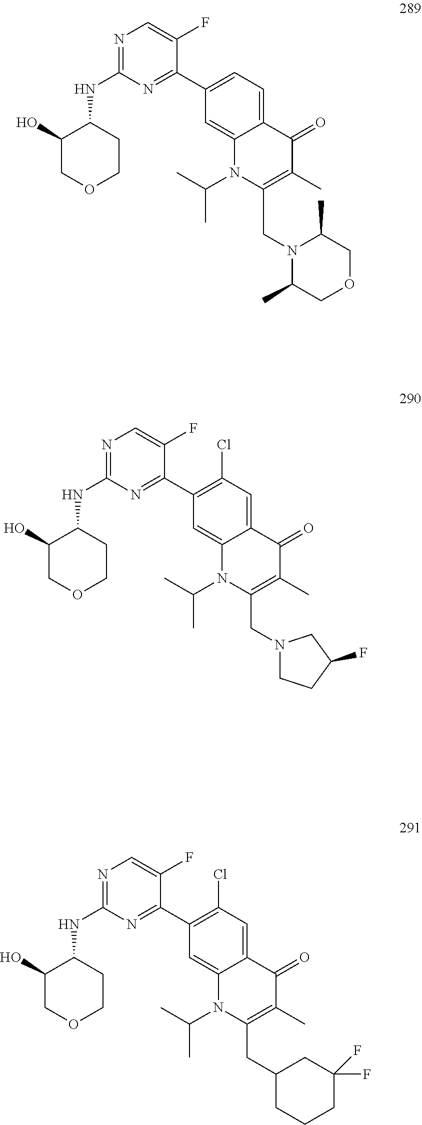 [SMILES: Cc1c(CN2[C@H](C)COC[C@@H]2C)n(C(C)C)c2cc(-c3nc(N[C@@H]4CCOC[C@H]4O)ncc3F)ccc2c1=O ; Cc1c(CN2CC[C@H](F)C2)n(C(C)C)c2cc(-c3nc(N[C@@H]4CCOC[C@H]4O)ncc3F)c(Cl)cc2c1=O ; Cc1c(CC2CCCC(F)(F)C2)n(C(C)C)c2cc(-c3nc(N[C@@H]4CCOC[C@H]4O)ncc3F)c(Cl)cc2c1=O]

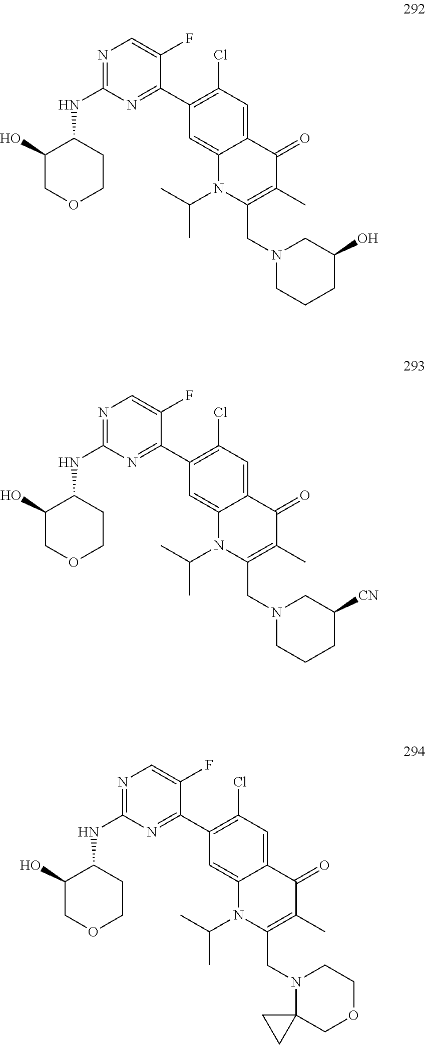 [SMILES: Cc1c(CN2CCC[C@H](O)C2)n(C(C)C)c2cc(-c3nc(N[C@@H]4CCOC[C@H]4O)ncc3F)c(Cl)cc2c1=O ; Cc1c(CN2CCC[C@H](C#N)C2)n(C(C)C)c2cc(-c3nc(N[C@@H]4CCOC[C@H]4O)ncc3F)c(Cl)cc2c1=O ; Cc1c(CN2CCOCC23CC3)n(C(C)C)c2cc(-c3nc(N[C@@H]4CCOC[C@H]4O)ncc3F)c(Cl)cc2c1=O]

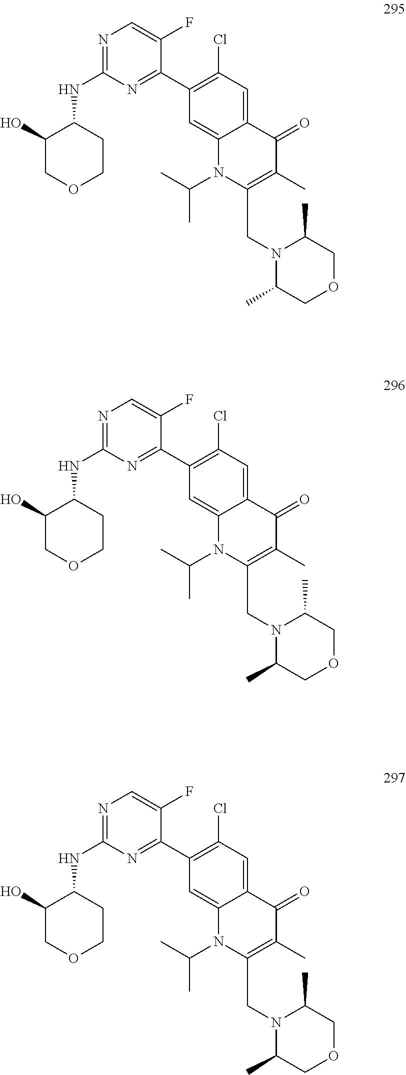 [SMILES: Cc1c(CN2[C@@H](C)COC[C@@H]2C)n(C(C)C)c2cc(-c3nc(N[C@@H]4CCOC[C@H]4O)ncc3F)c(Cl)cc2c1=O ; Cc1c(CN2[C@H](C)COC[C@H]2C)n(C(C)C)c2cc(-c3nc(N[C@@H]4CCOC[C@H]4O)ncc3F)c(Cl)cc2c1=O ; Cc1c(CN2[C@H](C)COC[C@@H]2C)n(C(C)C)c2cc(-c3nc(N[C@@H]4CCOC[C@H]4O)ncc3F)c(Cl)cc2c1=O]

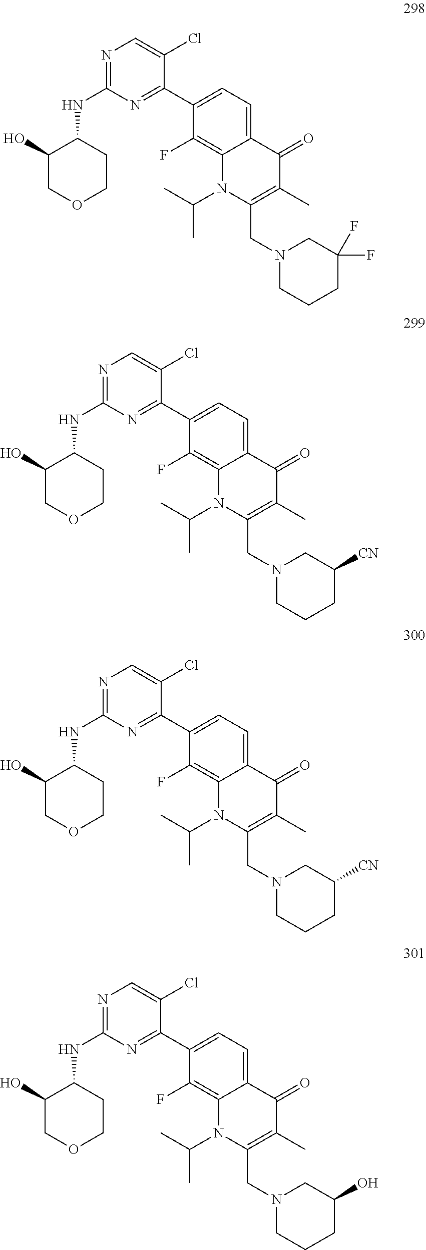 [SMILES: Cc1c(CN2CCCC(F)(F)C2)n(C(C)C)c2c(F)c(-c3nc(N[C@@H]4CCOC[C@H]4O)ncc3Cl)ccc2c1=O ; Cc1c(CN2CCC[C@H](C#N)C2)n(C(C)C)c2c(F)c(-c3nc(N[C@@H]4CCOC[C@H]4O)ncc3Cl)ccc2c1=O ; Cc1c(CN2CCC[C@@H](C#N)C2)n(C(C)C)c2c(F)c(-c3nc(N[C@@H]4CCOC[C@H]4O)ncc3Cl)ccc2c1=O ; Cc1c(CN2CCC[C@H](O)C2)n(C(C)C)c2c(F)c(-c3nc(N[C@@H]4CCOC[C@H]4O)ncc3Cl)ccc2c1=O]

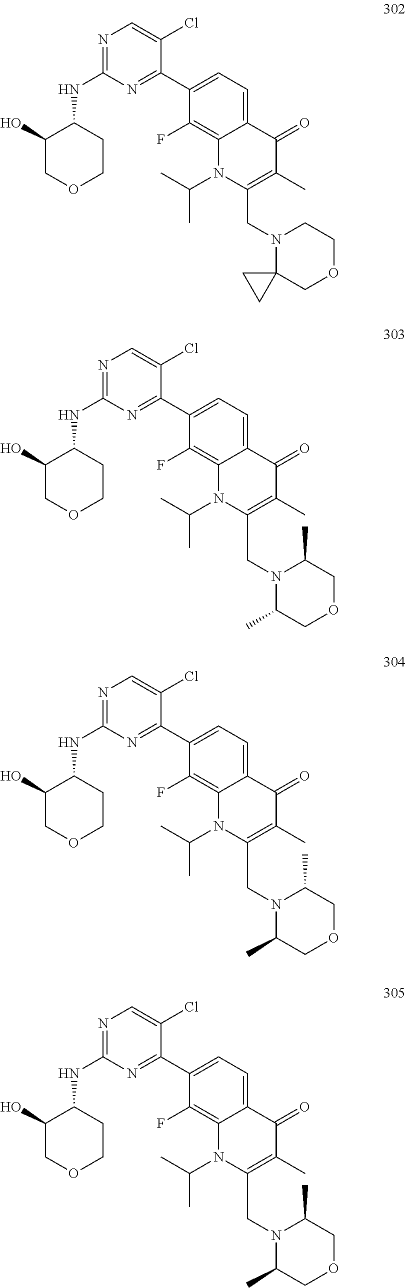 [SMILES: Cc1c(CN2CCOCC23CC3)n(C(C)C)c2c(F)c(-c3nc(N[C@@H]4CCOC[C@H]4O)ncc3Cl)ccc2c1=O ; Cc1c(CN2[C@@H](C)COC[C@@H]2C)n(C(C)C)c2c(F)c(-c3nc(N[C@@H]4CCOC[C@H]4O)ncc3Cl)ccc2c1=O ; Cc1c(CN2[C@H](C)COC[C@H]2C)n(C(C)C)c2c(F)c(-c3nc(N[C@@H]4CCOC[C@H]4O)ncc3Cl)ccc2c1=O ; Cc1c(CN2[C@H](C)COC[C@@H]2C)n(C(C)C)c2c(F)c(-c3nc(N[C@@H]4CCOC[C@H]4O)ncc3Cl)ccc2c1=O]

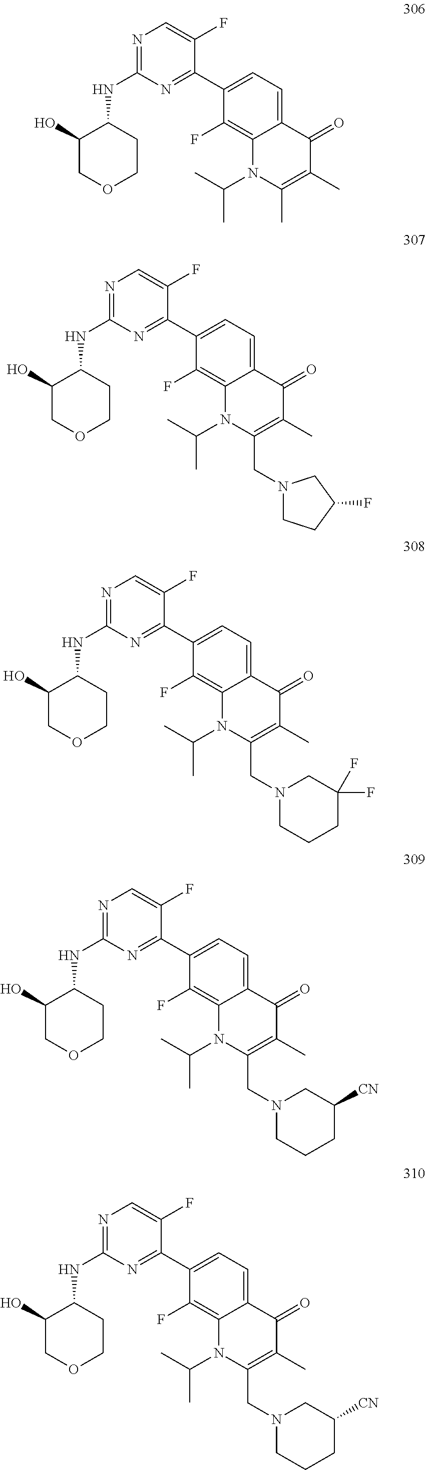 [SMILES: Cc1c(C)n(C(C)C)c2c(F)c(-c3nc(N[C@@H]4CCOC[C@H]4O)ncc3F)ccc2c1=O ; Cc1c(CN2CC[C@@H](F)C2)n(C(C)C)c2c(F)c(-c3nc(N[C@@H]4CCOC[C@H]4O)ncc3F)ccc2c1=O ; Cc1c(CN2CCCC(F)(F)C2)n(C(C)C)c2c(F)c(-c3nc(N[C@@H]4CCOC[C@H]4O)ncc3F)ccc2c1=O ; Cc1c(CN2CCC[C@H](C#N)C2)n(C(C)C)c2c(F)c(-c3nc(N[C@@H]4CCOC[C@H]4O)ncc3F)ccc2c1=O ; Cc1c(CN2CCC[C@@H](C#N)C2)n(C(C)C)c2c(F)c(-c3nc(N[C@@H]4CCOC[C@H]4O)ncc3F)ccc2c1=O]

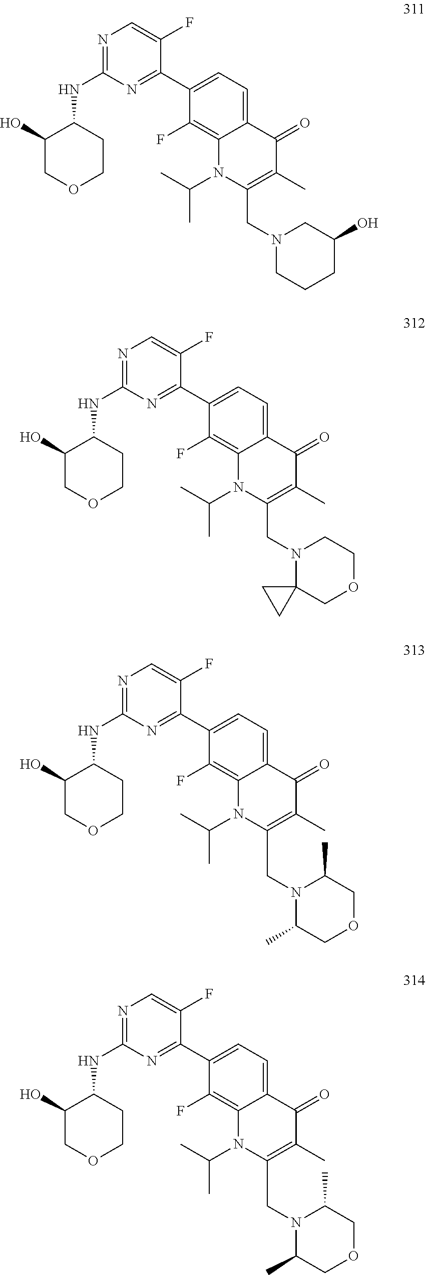 [SMILES: Cc1c(CN2CCC[C@H](O)C2)n(C(C)C)c2c(F)c(-c3nc(N[C@@H]4CCOC[C@H]4O)ncc3F)ccc2c1=O ; Cc1c(CN2CCOCC23CC3)n(C(C)C)c2c(F)c(-c3nc(N[C@@H]4CCOC[C@H]4O)ncc3F)ccc2c1=O ; Cc1c(CN2[C@@H](C)COC[C@@H]2C)n(C(C)C)c2c(F)c(-c3nc(N[C@@H]4CCOC[C@H]4O)ncc3F)ccc2c1=O ; Cc1c(CN2[C@H](C)COC[C@H]2C)n(C(C)C)c2c(F)c(-c3nc(N[C@@H]4CCOC[C@H]4O)ncc3F)ccc2c1=O]

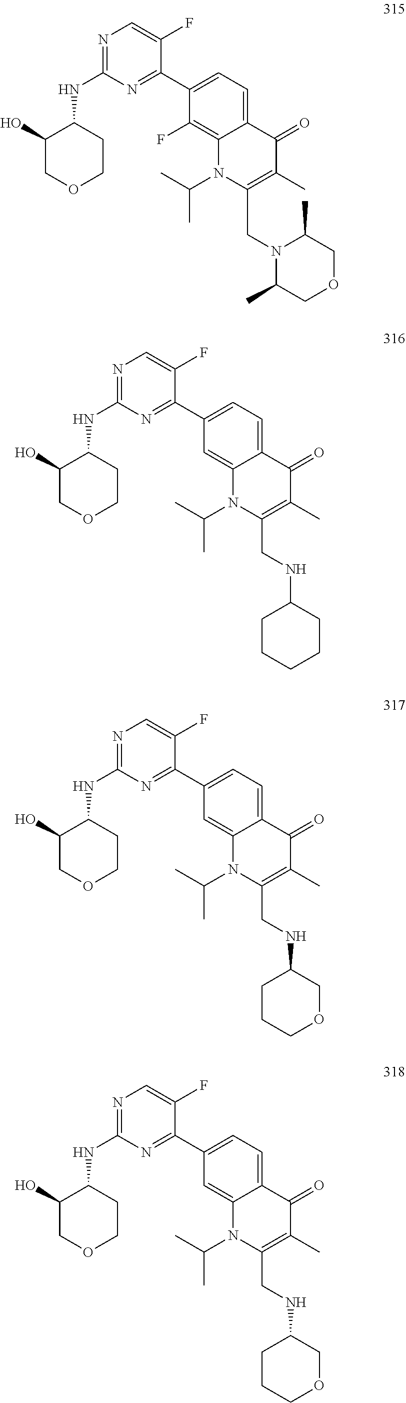 [SMILES: Cc1c(CN2[C@H](C)COC[C@@H]2C)n(C(C)C)c2c(F)c(-c3nc(N[C@@H]4CCOC[C@H]4O)ncc3F)ccc2c1=O ; Cc1c(CNC2CCCCC2)n(C(C)C)c2cc(-c3nc(N[C@@H]4CCOC[C@H]4O)ncc3F)ccc2c1=O ; Cc1c(CN[C@@H]2CCCOC2)n(C(C)C)c2cc(-c3nc(N[C@@H]4CCOC[C@H]4O)ncc3F)ccc2c1=O ; Cc1c(CN[C@H]2CCCOC2)n(C(C)C)c2cc(-c3nc(N[C@@H]4CCOC[C@H]4O)ncc3F)ccc2c1=O]

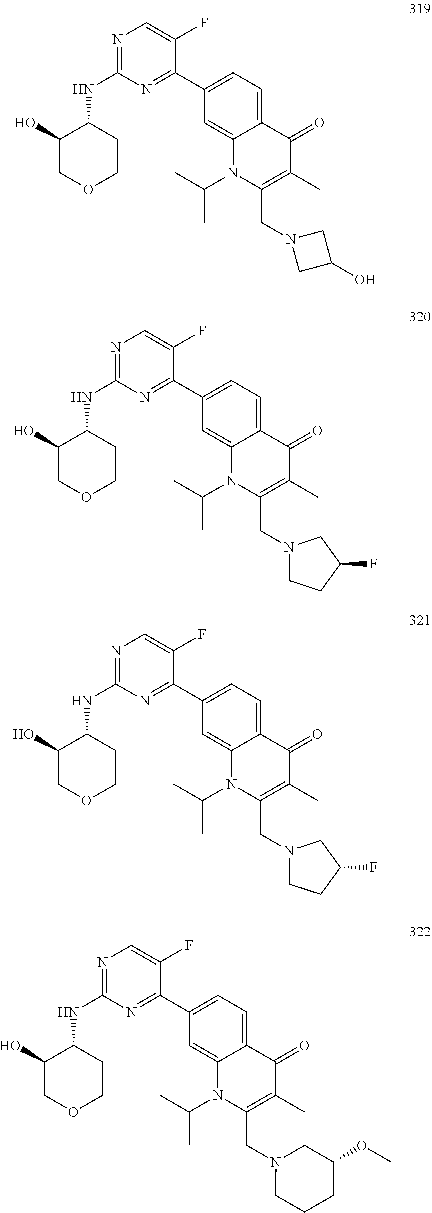 [SMILES: Cc1c(CN2CC(O)C2)n(C(C)C)c2cc(-c3nc(N[C@@H]4CCOC[C@H]4O)ncc3F)ccc2c1=O ; Cc1c(CN2CC[C@H](F)C2)n(C(C)C)c2cc(-c3nc(N[C@@H]4CCOC[C@H]4O)ncc3F)ccc2c1=O ; Cc1c(CN2CC[C@@H](F)C2)n(C(C)C)c2cc(-c3nc(N[C@@H]4CCOC[C@H]4O)ncc3F)ccc2c1=O ; CO[C@@H]1CCCN(Cc2c(C)c(=O)c3ccc(-c4nc(N[C@@H]5CCOC[C@H]5O)ncc4F)cc3n2C(C)C)C1]

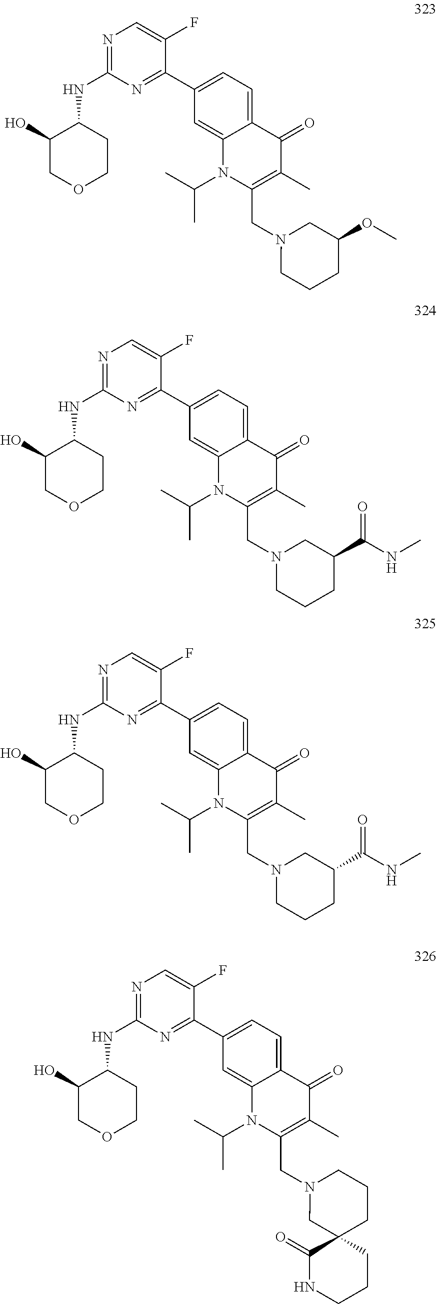 [SMILES: CO[C@H]1CCCN(Cc2c(C)c(=O)c3ccc(-c4nc(N[C@@H]5CCOC[C@H]5O)ncc4F)cc3n2C(C)C)C1 ; CNC(=O)[C@H]1CCCN(Cc2c(C)c(=O)c3ccc(-c4nc(N[C@@H]5CCOC[C@H]5O)ncc4F)cc3n2C(C)C)C1 ; CNC(=O)[C@@H]1CCCN(Cc2c(C)c(=O)c3ccc(-c4nc(N[C@@H]5CCOC[C@H]5O)ncc4F)cc3n2C(C)C)C1 ; Cc1c(CN2CCC[C@]3(CCCNC3=O)C2)n(C(C)C)c2cc(-c3nc(N[C@@H]4CCOC[C@H]4O)ncc3F)ccc2c1=O]

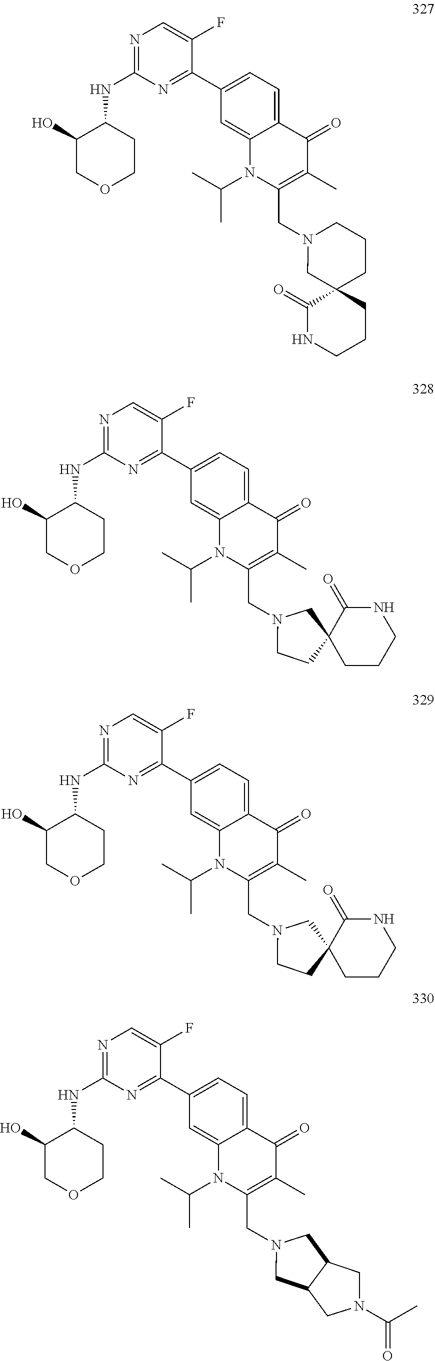

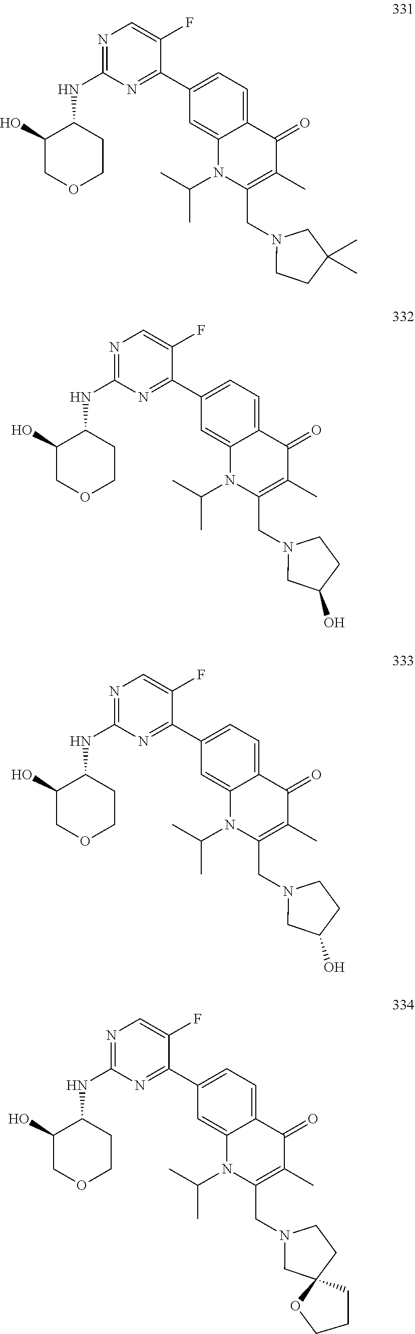 [SMILES: Cc1c(CN2CCC(C)(C)C2)n(C(C)C)c2cc(-c3nc(N[C@@H]4CCOC[C@H]4O)ncc3F)ccc2c1=O ; Cc1c(CN2CC[C@@H](O)C2)n(C(C)C)c2cc(-c3nc(N[C@@H]4CCOC[C@H]4O)ncc3F)ccc2c1=O ; Cc1c(CN2CC[C@H](O)C2)n(C(C)C)c2cc(-c3nc(N[C@@H]4CCOC[C@H]4O)ncc3F)ccc2c1=O ; Cc1c(CN2CC[C@]3(CCCO3)C2)n(C(C)C)c2cc(-c3nc(N[C@@H]4CCOC[C@H]4O)ncc3F)ccc2c1=O]

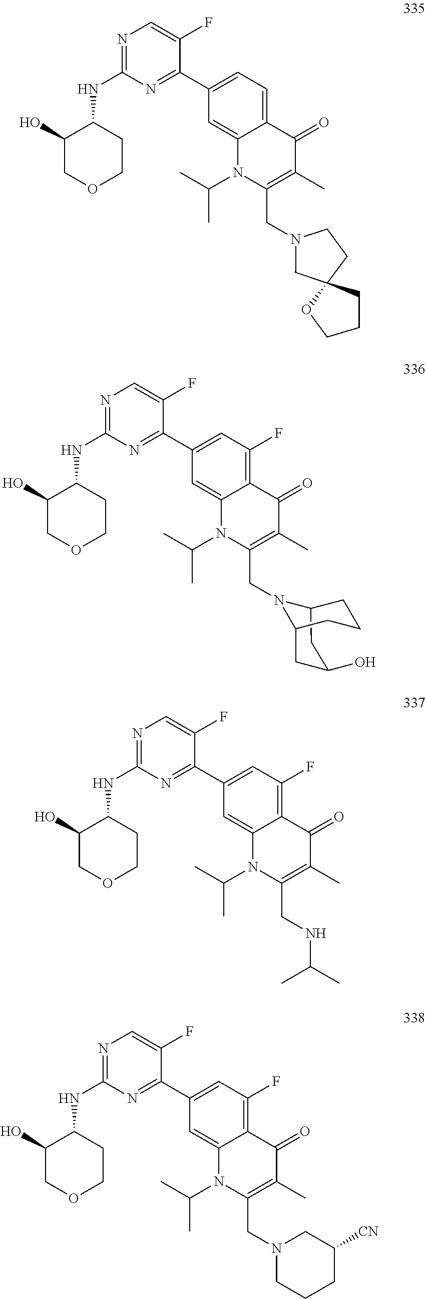 [SMILES: Cc1c(CN2CC[C@@]3(CCCO3)C2)n(C(C)C)c2cc(-c3nc(N[C@@H]4CCOC[C@H]4O)ncc3F)ccc2c1=O ; Cc1c(CN2C3CCCC2CC(O)C3)n(C(C)C)c2cc(-c3nc(N[C@@H]4CCOC[C@H]4O)ncc3F)cc(F)c2c1=O ; Cc1c(CNC(C)C)n(C(C)C)c2cc(-c3nc(N[C@@H]4CCOC[C@H]4O)ncc3F)cc(F)c2c1=O ; Cc1c(CN2CCC[C@@H](C#N)C2)n(C(C)C)c2cc(-c3nc(N[C@@H]4CCOC[C@H]4O)ncc3F)cc(F)c2c1=O]

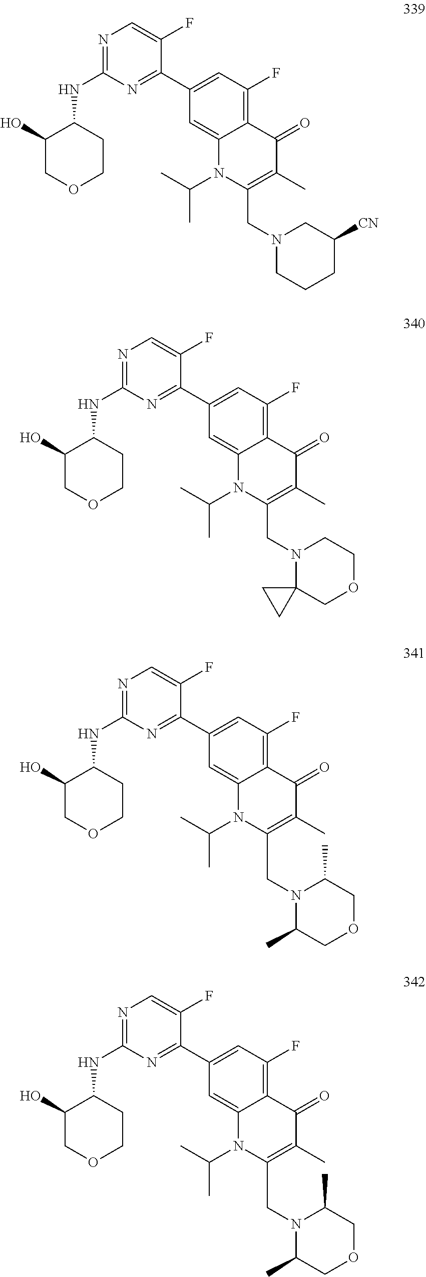 [SMILES: Cc1c(CN2CCC[C@H](C#N)C2)n(C(C)C)c2cc(-c3nc(N[C@@H]4CCOC[C@H]4O)ncc3F)cc(F)c2c1=O ; Cc1c(CN2CCOCC23CC3)n(C(C)C)c2cc(-c3nc(N[C@@H]4CCOC[C@H]4O)ncc3F)cc(F)c2c1=O ; Cc1c(CN2[C@H](C)COC[C@H]2C)n(C(C)C)c2cc(-c3nc(N[C@@H]4CCOC[C@H]4O)ncc3F)cc(F)c2c1=O ; Cc1c(CN2[C@H](C)COC[C@@H]2C)n(C(C)C)c2cc(-c3nc(N[C@@H]4CCOC[C@H]4O)ncc3F)cc(F)c2c1=O]

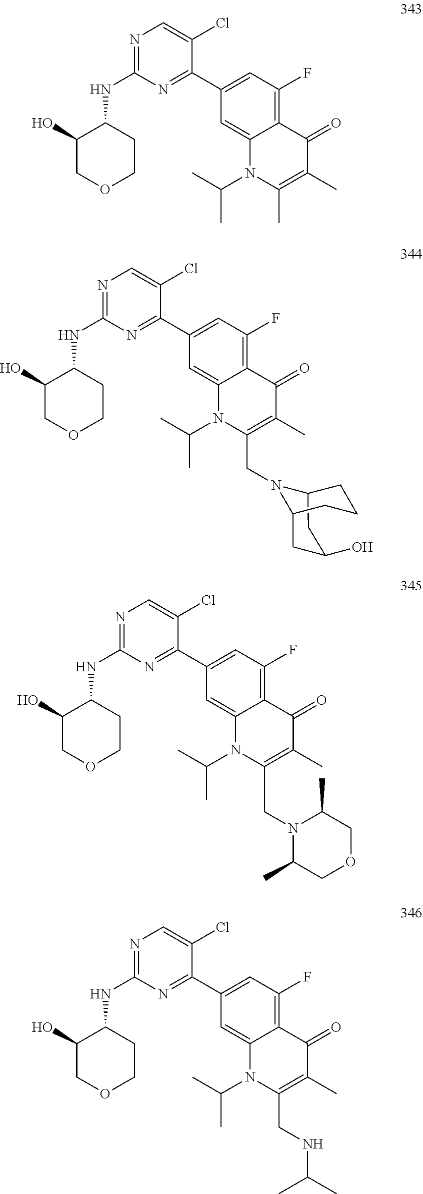 [SMILES: Cc1c(C)n(C(C)C)c2cc(-c3nc(N[C@@H]4CCOC[C@H]4O)ncc3Cl)cc(F)c2c1=O ; Cc1c(CN2C3CCCC2CC(O)C3)n(C(C)C)c2cc(-c3nc(N[C@@H]4CCOC[C@H]4O)ncc3Cl)cc(F)c2c1=O ; Cc1c(CN2[C@H](C)COC[C@@H]2C)n(C(C)C)c2cc(-c3nc(N[C@@H]4CCOC[C@H]4O)ncc3Cl)cc(F)c2c1=O ; Cc1c(CNC(C)C)n(C(C)C)c2cc(-c3nc(N[C@@H]4CCOC[C@H]4O)ncc3Cl)cc(F)c2c1=O]

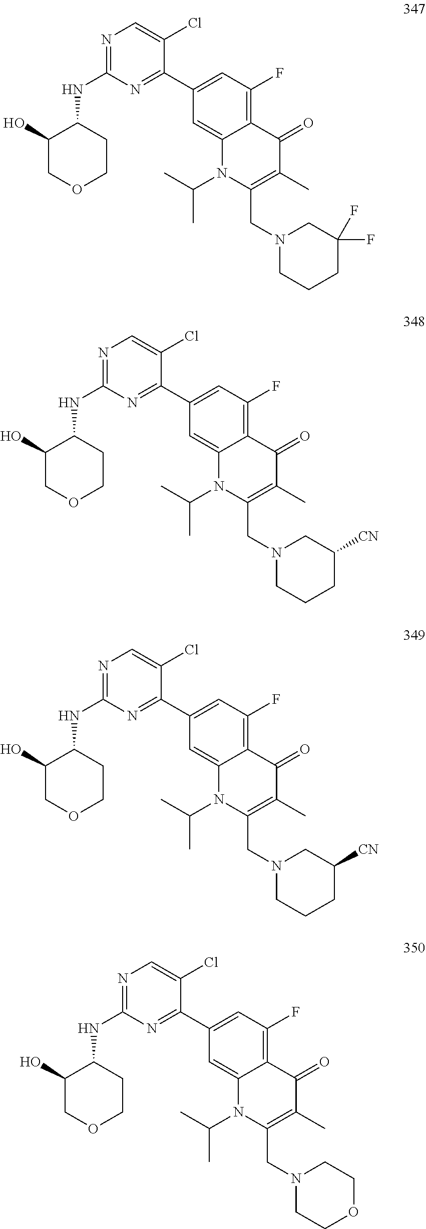 [SMILES: Cc1c(CN2CCCC(F)(F)C2)n(C(C)C)c2cc(-c3nc(N[C@@H]4CCOC[C@H]4O)ncc3Cl)cc(F)c2c1=O ; Cc1c(CN2CCC[C@@H](C#N)C2)n(C(C)C)c2cc(-c3nc(N[C@@H]4CCOC[C@H]4O)ncc3Cl)cc(F)c2c1=O ; Cc1c(CN2CCC[C@H](C#N)C2)n(C(C)C)c2cc(-c3nc(N[C@@H]4CCOC[C@H]4O)ncc3Cl)cc(F)c2c1=O ; Cc1c(CN2CCOCC2)n(C(C)C)c2cc(-c3nc(N[C@@H]4CCOC[C@H]4O)ncc3Cl)cc(F)c2c1=O]

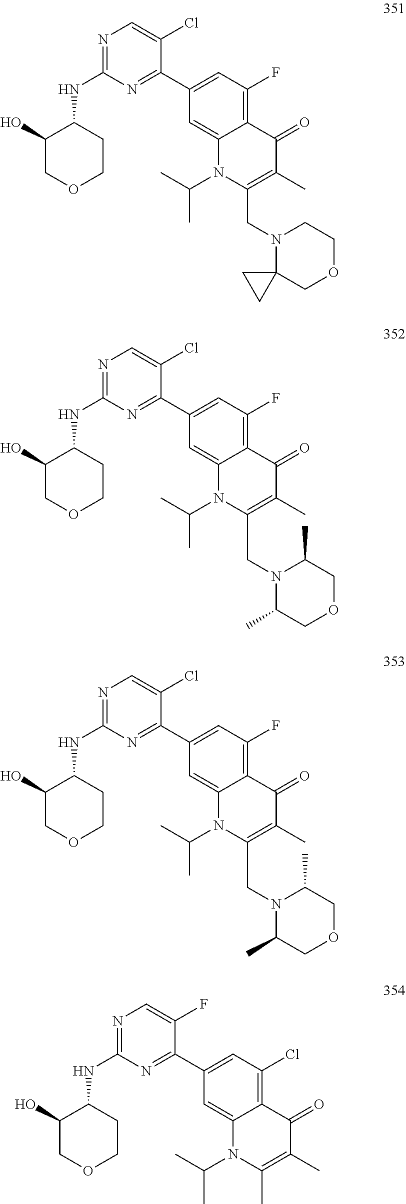 [SMILES: Cc1c(CN2CCOCC23CC3)n(C(C)C)c2cc(-c3nc(N[C@@H]4CCOC[C@H]4O)ncc3Cl)cc(F)c2c1=O ; Cc1c(CN2[C@@H](C)COC[C@@H]2C)n(C(C)C)c2cc(-c3nc(N[C@@H]4CCOC[C@H]4O)ncc3Cl)cc(F)c2c1=O ; Cc1c(CN2[C@H](C)COC[C@H]2C)n(C(C)C)c2cc(-c3nc(N[C@@H]4CCOC[C@H]4O)ncc3Cl)cc(F)c2c1=O ; Cc1c(C)n(C(C)C)c2cc(-c3nc(N[C@@H]4CCOC[C@H]4O)ncc3F)cc(Cl)c2c1=O]

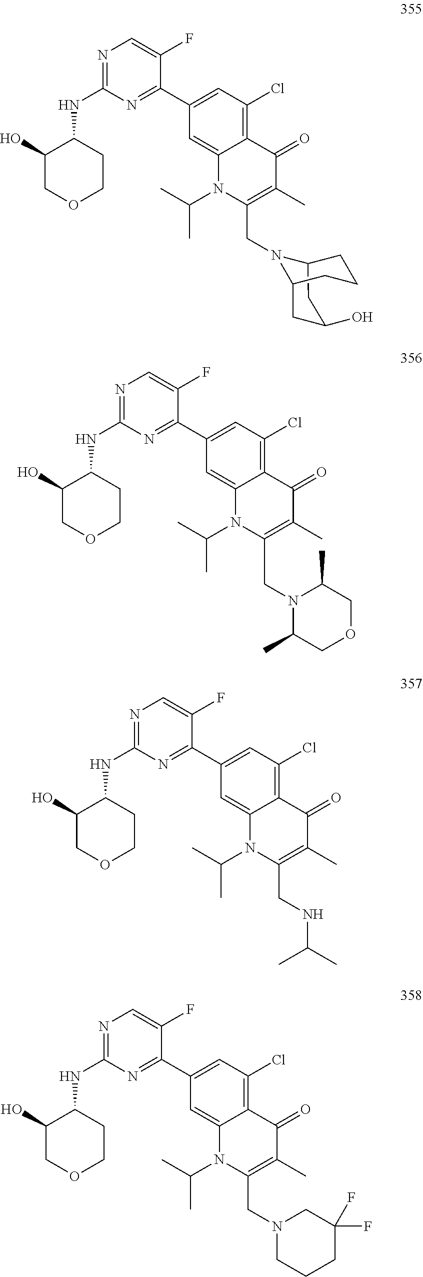 [SMILES: Cc1c(CN2C3CCCC2CC(O)C3)n(C(C)C)c2cc(-c3nc(N[C@@H]4CCOC[C@H]4O)ncc3F)cc(Cl)c2c1=O ; Cc1c(CN2[C@H](C)COC[C@@H]2C)n(C(C)C)c2cc(-c3nc(N[C@@H]4CCOC[C@H]4O)ncc3F)cc(Cl)c2c1=O ; Cc1c(CNC(C)C)n(C(C)C)c2cc(-c3nc(N[C@@H]4CCOC[C@H]4O)ncc3F)cc(Cl)c2c1=O ; Cc1c(CN2CCCC(F)(F)C2)n(C(C)C)c2cc(-c3nc(N[C@@H]4CCOC[C@H]4O)ncc3F)cc(Cl)c2c1=O]

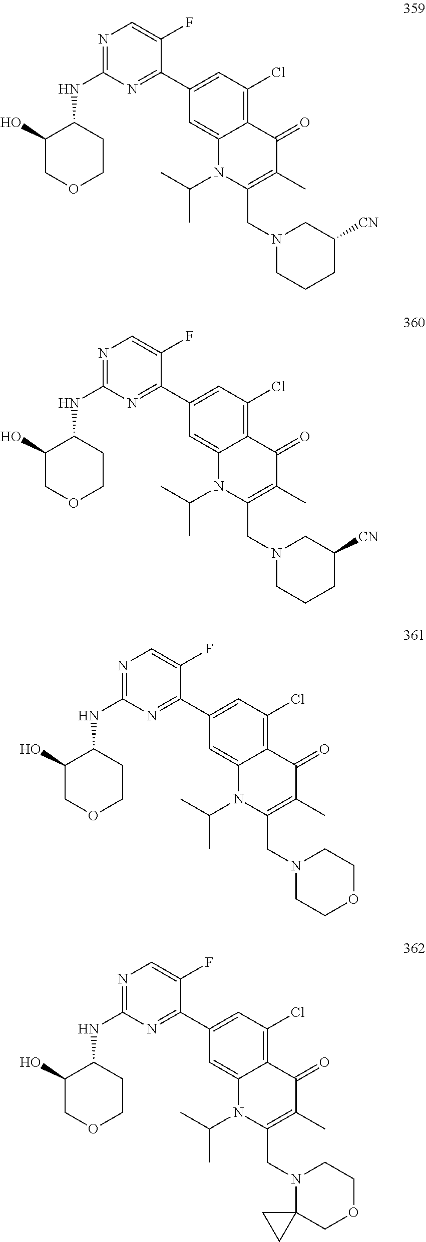 [SMILES: Cc1c(CN2CCC[C@@H](C#N)C2)n(C(C)C)c2cc(-c3nc(N[C@@H]4CCOC[C@H]4O)ncc3F)cc(Cl)c2c1=O ; Cc1c(CN2CCC[C@H](C#N)C2)n(C(C)C)c2cc(-c3nc(N[C@@H]4CCOC[C@H]4O)ncc3F)cc(Cl)c2c1=O ; Cc1c(CN2CCOCC2)n(C(C)C)c2cc(-c3nc(N[C@@H]4CCOC[C@H]4O)ncc3F)cc(Cl)c2c1=O ; Cc1c(CN2CCOCC23CC3)n(C(C)C)c2cc(-c3nc(N[C@@H]4CCOC[C@H]4O)ncc3F)cc(Cl)c2c1=O]

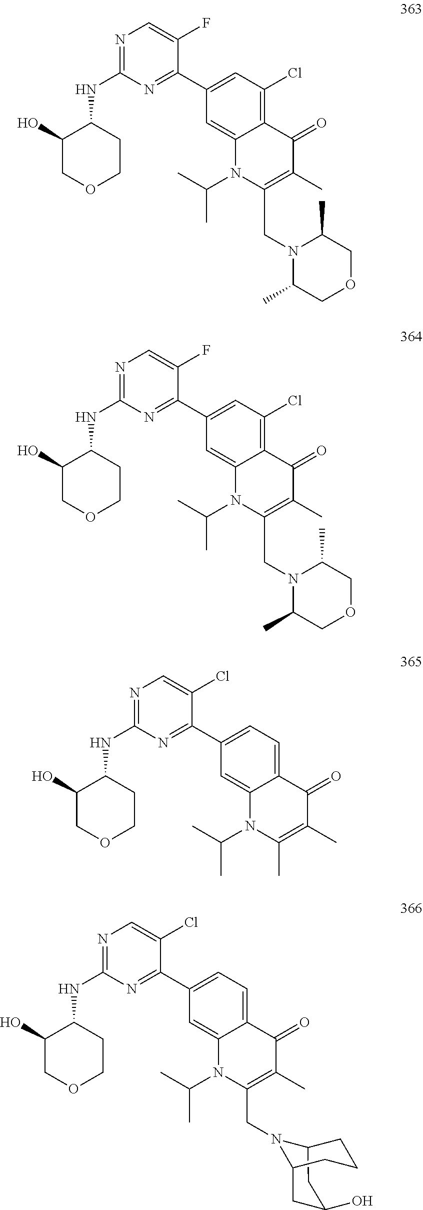 [SMILES: Cc1c(CN2[C@@H](C)COC[C@@H]2C)n(C(C)C)c2cc(-c3nc(N[C@@H]4CCOC[C@H]4O)ncc3F)cc(Cl)c2c1=O ; Cc1c(CN2[C@H](C)COC[C@H]2C)n(C(C)C)c2cc(-c3nc(N[C@@H]4CCOC[C@H]4O)ncc3F)cc(Cl)c2c1=O ; Cc1c(C)n(C(C)C)c2cc(-c3nc(N[C@@H]4CCOC[C@H]4O)ncc3Cl)ccc2c1=O ; Cc1c(CN2C3CCCC2CC(O)C3)n(C(C)C)c2cc(-c3nc(N[C@@H]4CCOC[C@H]4O)ncc3Cl)ccc2c1=O]

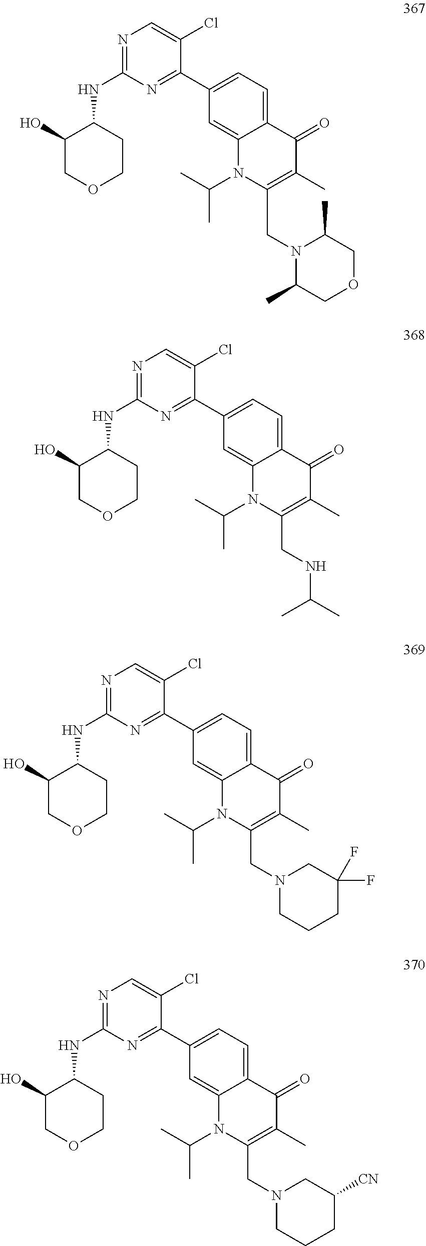 [SMILES: Cc1c(CN2[C@H](C)COC[C@@H]2C)n(C(C)C)c2cc(-c3nc(N[C@@H]4CCOC[C@H]4O)ncc3Cl)ccc2c1=O ; Cc1c(CNC(C)C)n(C(C)C)c2cc(-c3nc(N[C@@H]4CCOC[C@H]4O)ncc3Cl)ccc2c1=O ; Cc1c(CN2CCCC(F)(F)C2)n(C(C)C)c2cc(-c3nc(N[C@@H]4CCOC[C@H]4O)ncc3Cl)ccc2c1=O ; Cc1c(CN2CCC[C@@H](C#N)C2)n(C(C)C)c2cc(-c3nc(N[C@@H]4CCOC[C@H]4O)ncc3Cl)ccc2c1=O]

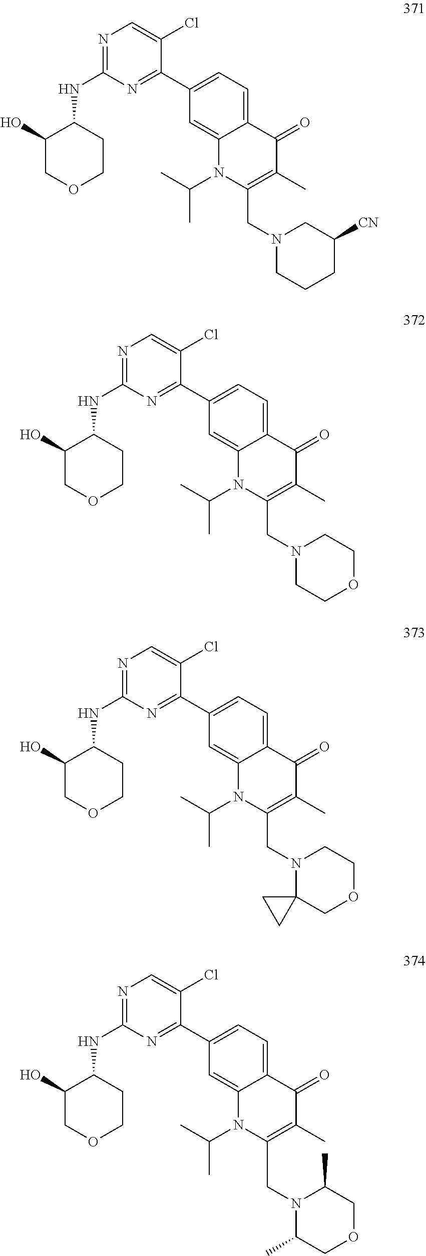 [SMILES: Cc1c(CN2CCC[C@H](C#N)C2)n(C(C)C)c2cc(-c3nc(N[C@@H]4CCOC[C@H]4O)ncc3Cl)ccc2c1=O ; Cc1c(CN2CCOCC2)n(C(C)C)c2cc(-c3nc(N[C@@H]4CCOC[C@H]4O)ncc3Cl)ccc2c1=O ; Cc1c(CN2CCOCC23CC3)n(C(C)C)c2cc(-c3nc(N[C@@H]4CCOC[C@H]4O)ncc3Cl)ccc2c1=O ; Cc1c(CN2[C@@H](C)COC[C@@H]2C)n(C(C)C)c2cc(-c3nc(N[C@@H]4CCOC[C@H]4O)ncc3Cl)ccc2c1=O]

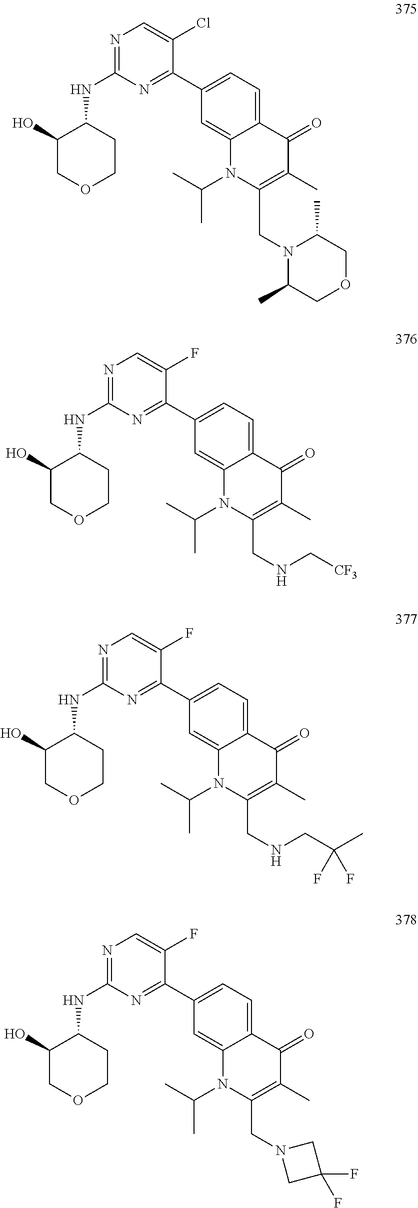 [SMILES: Cc1c(CN2[C@H](C)COC[C@H]2C)n(C(C)C)c2cc(-c3nc(N[C@@H]4CCOC[C@H]4O)ncc3Cl)ccc2c1=O ; Cc1c(CNCC(F)(F)F)n(C(C)C)c2cc(-c3nc(N[C@@H]4CCOC[C@H]4O)ncc3F)ccc2c1=O ; Cc1c(CNCC(C)(F)F)n(C(C)C)c2cc(-c3nc(N[C@@H]4CCOC[C@H]4O)ncc3F)ccc2c1=O ; Cc1c(CN2CC(F)(F)C2)n(C(C)C)c2cc(-c3nc(N[C@@H]4CCOC[C@H]4O)ncc3F)ccc2c1=O]

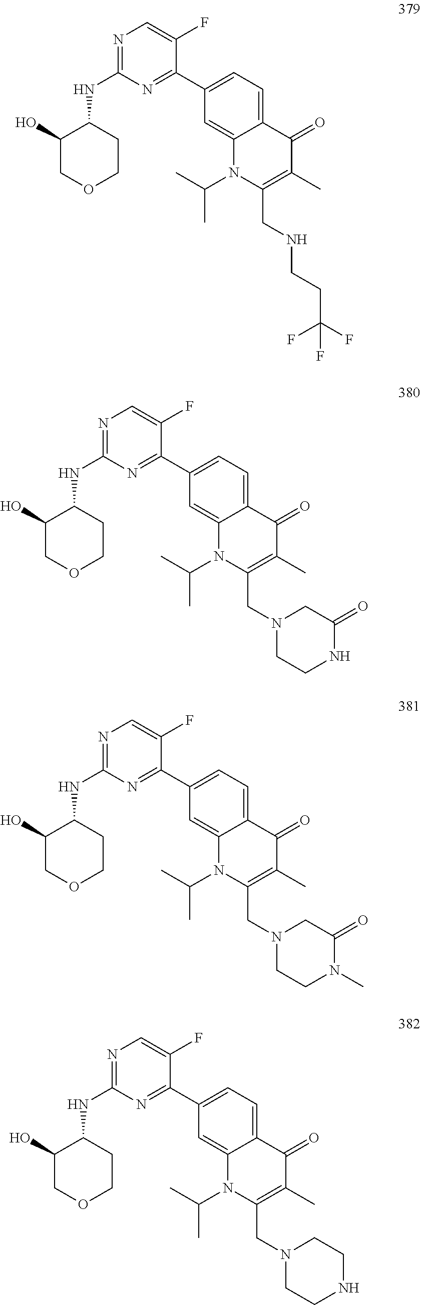 [SMILES: Cc1c(CNCCC(F)(F)F)n(C(C)C)c2cc(-c3nc(N[C@@H]4CCOC[C@H]4O)ncc3F)ccc2c1=O ; Cc1c(CN2CCNC(=O)C2)n(C(C)C)c2cc(-c3nc(N[C@@H]4CCOC[C@H]4O)ncc3F)ccc2c1=O ; Cc1c(CN2CCN(C)C(=O)C2)n(C(C)C)c2cc(-c3nc(N[C@@H]4CCOC[C@H]4O)ncc3F)ccc2c1=O ; Cc1c(CN2CCNCC2)n(C(C)C)c2cc(-c3nc(N[C@@H]4CCOC[C@H]4O)ncc3F)ccc2c1=O]

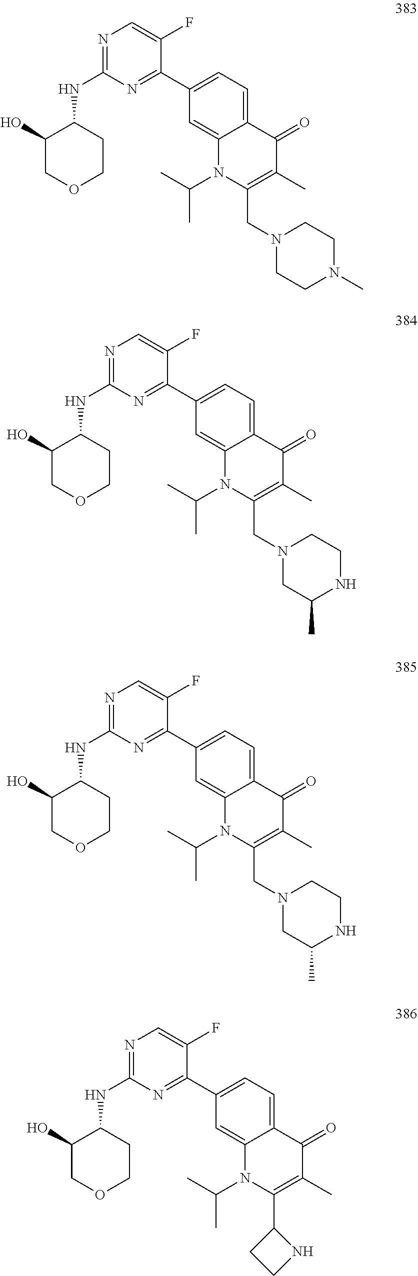 [SMILES: Cc1c(CN2CCN(C)CC2)n(C(C)C)c2cc(-c3nc(N[C@@H]4CCOC[C@H]4O)ncc3F)ccc2c1=O ; Cc1c(CN2CCN[C@@H](C)C2)n(C(C)C)c2cc(-c3nc(N[C@@H]4CCOC[C@H]4O)ncc3F)ccc2c1=O ; Cc1c(CN2CCN[C@H](C)C2)n(C(C)C)c2cc(-c3nc(N[C@@H]4CCOC[C@H]4O)ncc3F)ccc2c1=O ; Cc1c(C2CCN2)n(C(C)C)c2cc(-c3nc(N[C@@H]4CCOC[C@H]4O)ncc3F)ccc2c1=O]

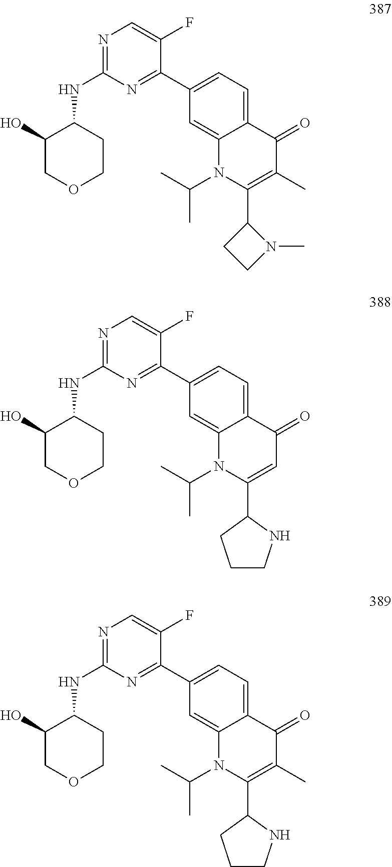 [SMILES: Cc1c(C2CCN2C)n(C(C)C)c2cc(-c3nc(N[C@@H]4CCOC[C@H]4O)ncc3F)ccc2c1=O ; CC(C)n1c(C2CCCN2)cc(=O)c2ccc(-c3nc(N[C@@H]4CCOC[C@H]4O)ncc3F)cc21 ; Cc1c(C2CCCN2)n(C(C)C)c2cc(-c3nc(N[C@@H]4CCOC[C@H]4O)ncc3F)ccc2c1=O]

Aspect 20. A pharmaceutical composition comprising a compound of any one of Aspects 1-19 or a pharmaceutically acceptable salt, stereoisomer, tautomer or prodrug thereof, together with a pharmaceutically acceptable excipient.

Aspect 21. A method of decreasing CDK4 activity by inhibition, which comprises administering to an individual the compound according to any one of Aspects 1-19, or a pharmaceutically acceptable salt thereof, including the compound of formula (I) or the specific compounds exemplified herein.

Aspect 22. The method of Aspect 21, wherein the disease is selected from cancer, preferred breast cancer, lung cancer, pancreatic cancer, prostate cancer, bone cancer, liver cancer and endometrial cancer.

Aspect 23. Use of a compound of any one of Aspects 1-19 or a pharmaceutically acceptable salt, stereoisomer, tautomer or prodrug thereof in the preparation of a medicament for treating a disease that can be affected by CDK4 modulation.

Aspect 24. The use of Aspect 23, wherein the disease is cancer, preferred breast cancer, lung cancer, pancreatic cancer, prostate cancer, bone cancer, liver cancer and endometrial cancer.

Aspect 25. A method of treating a disease or disorder in a patient comprising administering to the patient a therapeutically effective amount of the compound any one of Aspects 1-19, or a pharmaceutically acceptable salt thereof as a CKD4 kinase inhibitor, wherein the disease or disorder is associated with inhibition of CDK4.

Aspect 26. The method of Aspect 25, wherein the disease is selected from cancer, preferred breast cancer, lung cancer, pancreatic cancer, prostate cancer, bone cancer, liver cancer and endometrial cancer.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C═C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2,3-dihydro-1H-indenyl, 1H-indenyl, 1,2,3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein.

Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed Drug Stereochemistry: Analytical Methods and Pharmacology. New York: Marcel Dekker. Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### EXAMPLES

### General Synthesis

Compounds disclosed herein, including salts thereof, can be prepared using known organic synthesis techniques and can be synthesized according to any of numerous possible synthetic routes. The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the boiling temperature of solvent. A given reaction can be carried out in one solvent or mixture of solvents.

The selection of appropriate protecting group, can be readily determined by one skilled in the art.

Reactions can be monitored according to any suitable method known in the art, such as NMR. UV. HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ration used.

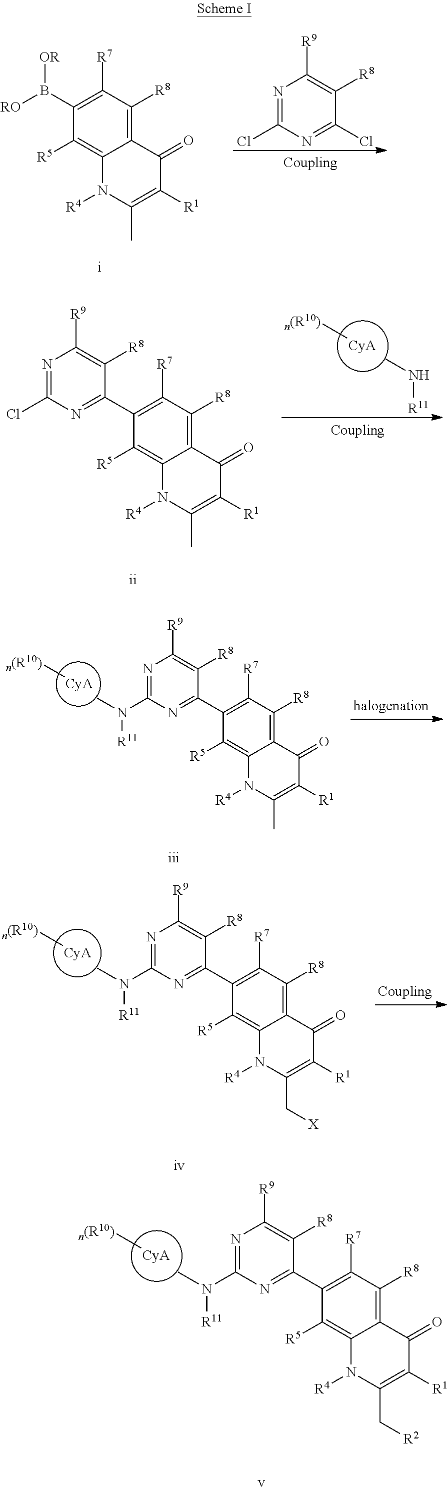

For example, compounds of Formulas (I), (II), (III), or (IV) can be formed as shown in Scheme I. The compound (i) can react with halogenated pyrimidine under palladium catalyzed reaction condition or base mediated coupling condition to give compound (ii) that can couple with amine to give compound (iii), halogenation of compound (iii) give compound (iv) which can be used for coupling to give compound (v).

### ABBREVIATIONS

NMR nuclear magnetic resonance
UV ultraviolet
HPLC high performance liquid chromatography
LC-MS liquid chromatograph mass spectrometer
TLC thin layer chromatography
PE petroleum ether
Et ethyl
Ac acetyl
DCM dichloromethane
Me methyl
DMSO dimethyl sulfoxide
Boc tert-butyloxycarbonyl
dppf 1,1′-bis(diphenylphosphino)ferrocene
BPD bis(pinacolato)diboron
Bu butyl
m-CPBA meta-chloroperbenzoic acid
dba dibenzylideneacetone
DMF N,N-dimethylformamide
THF tetrahydrofuran
dtbpf 1,1′-bis(di-tert-butylphosphino)ferrocene
NBS N-bromosuccinimide
NCS N-iodosuccinimide
DCE dichloroethane
Ts p-toluenesulfonyl
MTBE methyl tert-butyl ether
TR-FRET time-resolved fluorescence resonance energy transfer
tris-HCl tris(hydroxymethyl)aminomethane hydrochloride
BSA bovine serum albumin
TCEP tris(2-carboxyethyl)phosphine

### Example 1: 5-chloro-7-(5-chloro-2-(((3S,4R)-3-hydroxytetrahydro-2H-pyran-4-yl)amino)pyrimidin-4-yl)-1-isopropylquinolin-4(1H)-one

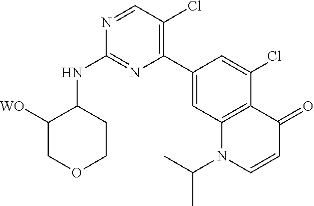

### Step 1: 4-bromo-2-chloro-6-fluorobenzoyl chloride

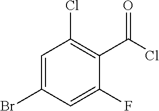 [SMILES: O=C(Cl)c1c(F)cc(Br)cc1Cl]

4-Bromo-2-chloro-6-fluorobenzoic acid (6 g, 23.7 mmol) were dissolved in dry dichloromethane (60 mL) before addition of dimethylformamide (0.5 mL). Oxalyl dichloride (3 mL, 35.5 mmol) was added dropwise at 0° C. and the reaction solution was stirred at room temperature for 1.5 h. Solvents were removed under reduced pressure. The crude title compound (6.44 g, 100%) was obtained without further purification.

### Step 2: ethyl (Z)-2-(4-bromo-2-chloro-6-fluorobenzoyl)-3-(isopropylamino)acrylate

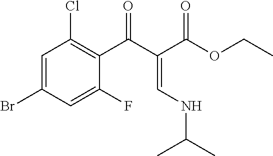 [SMILES: CCOC(=O)/C(=C\NC(C)C)C(=O)c1c(F)cc(Br)cc1Cl]

To a solution of 4-bromo-2-chloro-6-fluorobenzoyl chloride (6.44 g, 23.7 mmol) and ethyl (E)-3-(dimethylamino)acrylate (5.1 g, 35.5 mol) in toluene (100 mL) was added triethylamine (4.9 mL, 35.5 mmol) and the solution was refluxed for overnight. After the reaction solution was cooled to room temperature, triethylamine (8 g, 79.2 mmol) and propan-2-amine hydrochloride (4.54 g, 47.5 mmol) were added. The resulting mixture was heated to 50° C. and stirred at 50° C. for 2 h before cooled to room temperature. Water (20 mL) was added and the aqueous layer was extracted with ethyl acetate (80 mL×3). The combine organic layers were washed with brine, dried over sodium sulfate, decanted and concentrated. The residue was purified over silica gel by column chromatography to give the title compound (7 g, 100%). LC-MS (M+H)⁺=392.0.

### Step 3: ethyl 7-bromo-5-chloro-1-isopropyl-4-oxo-1,4-dihydroquinoline-3-carboxylate

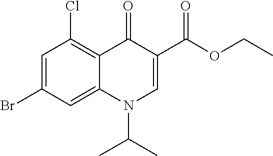 [SMILES: CCOC(=O)c1cn(C(C)C)c2cc(Br)cc(Cl)c2c1=O]

Ethyl (Z)-2-(4-bromo-2-chloro-6-fluorobenzoyl)-3-(isopropylamino)acrylate (7 g, 17.83 mmol) and potassium carbonate (3.7 g, 26.74 mmol) were dissolved in dimethylformamide (100 mL) and the reaction mixture was stirred at 60° C. for 14 h before cooled to room temperature. Solvent was removed under reduced pressure before addition of water (20 mL). The aqueous layer was extracted with ethyl acetate. The combine organic layers were washed with brine, dried over sodium sulfate, filtered and concentrated. The residue was purified over silica gel by column chromatography to give the title compound (2.66 g, 40%). LC-MS (M+H)⁺=372.0.

### Step 4: 7-bromo-5-chloro-1-isopropyl-4-oxo-1,4-dihydroquinoline-3-carboxylic acid

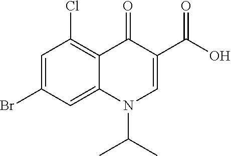 [SMILES: CC(C)n1cc(C(=O)O)c(=O)c2c(Cl)cc(Br)cc21]

To ethyl 7-bromo-5-chloro-1-isopropyl-4-oxo-1,4-dihydroquinoline-3-carboxylate (359 mg, 0.964 mmol) in ethanol (20 mL) was added 6N HCl (20 mL) and the resulting mixture was heated to reflux for 14 h before cooled to room temperature. The mixture was filtered and the crude product (290 mg, 87%) was used for next step without further purification. LC-MS (M+H)⁺=344.0.

### Step 5: 7-bromo-5-chloro-1-isopropylquinolin-4(1H)-one

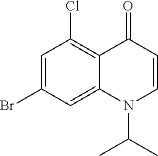 [SMILES: CC(C)n1ccc(=O)c2c(Cl)cc(Br)cc21]

A solution of 7-bromo-5-chloro-1-isopropyl-4-oxo-1,4-dihydroquinoline-3-carboxylic acid (290 mg, 0.84 mmol) was dissolved in diphenyl ether (5 mL) was stirred at 260° C. for 14 h before cooled to room temperature and concentrated. The residue was purified over silica gel by column chromatography to give the title compound (244 mg, 96%). LC-MS (M+H)⁺=300.0.

### Step 6: 5-chloro-1-isopropyl-7-(4,4,5,5-tetramethyl-1,3,2-dioxaborolan-2-yl)quinolin-4(1H)-one

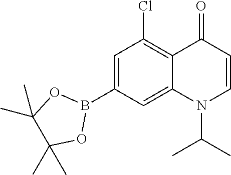 [SMILES: CC(C)n1ccc(=O)c2c(Cl)cc(B3OC(C)(C)C(C)(C)O3)cc21]

A mixture of 7-bromo-5-chloro-1-isopropylquinolin-4(1H)-one (244 mg, 0.812 mmol), 4,4,4,4′,5,5,5′,5′-octamethyl-2,2′-bi(1,3,2-dioxaborolane) (268 mg, 1.06 mmol), potassium acetate (119.5 mg, 1.22 mmol) and [1,1′-bis(diphenylphosphino)ferrocene]dichloropalladium(II) (59.4 mg, 0.0812 mmol) in 1,4-dioxane (10 mL) was stirred at 95° C. under nitrogen for 14 h before cooled to room temperature and concentrated. Water (20 mL) was added and the aqueous mixture was extracted with ethyl acetate (40 mL×3). The combined organic layers were washed with brine, dried over sodium sulfate, filtered and concentrated. The residue was purified over silica gel by column chromatography to give the title compound (282.2 mg, 100%). LC-MS (M+H)⁺=348.0.

### Step 7: 5-chloro-7-(2,5-dichloropyrimidin-4-yl)-1-isopropylquinolin-4(1H)-one

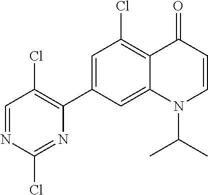 [SMILES: CC(C)n1ccc(=O)c2c(Cl)cc(-c3nc(Cl)ncc3Cl)cc21]

A mixture of 2,4,5-trichloropyrimidine (148.9 mg, 0.812 mmol), 5-chloro-1-isopropyl-7-(4,4,5,5-tetramethyl-1,3,2-dioxaborolan-2-yl)quinolin-4(1H)-one (282.2 mg, 0.812 mmol), sodium carbonate (258 mg, 2.436 mmol) and tetrakis(triphenylphosphine)palladium(0) (94 mg, 0.0812 mmol) in 1,4-dioxane (10 mL) and water (2 mL) was stirred at 90° C. under N₂ for 1.5 h before cooled to room temperature. Solvents were removed under reduced pressure. Water (20 mL) was added and the aqueous layer was extracted with ethyl acetate (50 mL×3). The combined organic layers were washed with brine, dried over sodium sulfate, filtered and concentrated. The residue was purified over silica gel by column chromatography to give the title compound (207 mg, 69%). LC-MS (M+H)⁺=368.0.

### Step 8: 5-chloro-7-(5-chloro-2-(((3S,4R)-3-hydroxytetrahydro-2H-pyran-4-yl)amino)pyrimidin-4-yl)-1-isopropylquinolin-4(1H)-one

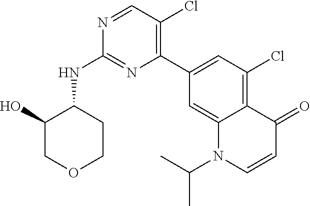 [SMILES: CC(C)n1ccc(=O)c2c(Cl)cc(-c3nc(N[C@@H]4CCOC[C@H]4O)ncc3Cl)cc21]

A mixture of 5-chloro-7-(2,5-dichloropyrimidin-4-yl)-1-isopropylquinolin-4(1H)-one (207 mg, 0.56 mmol), (3S,4R)-4-aminotetrahydro-2H-pyran-3-ol hydrochloride (129 mg, 0.84 mmol) and N,N-diisopropylethylamine (217.9 mg, 1.69 mmole) in acetonitrile (10 mL) was heated to reflux for 2 d before cooled to room temperature and concentrated. Water (5 mL) was added and the aqueous layer was extracted with ethyl acetate (30 mL×3). The combined organic layers were washed with brine, dried over sodium sulfate, filtered and concentrated. The residue was purified over silica gel by column chromatography to give the title compound (40 mg, 16%). ¹H-NMR (400 MHz, DMSO-d6) δ 8.48 (s, 1H), 8.14 (s, 1H), 8.10 (d, J=8.0 Hz, 1H), 7.63 (s, 2H), 6.15 (d, J=8.0 Hz, 1H), 5.05-4.95 (m, 1H), 4.93 (d, J=5.3 Hz, 1H), 3.91-3.74 (m, 3H), 3.50 (s, 1H), 3.31-3.25 (m, 1H), 3.04 (d, J=9.6 Hz, 1H), 1.95 (s, 1H), 1.46 (d, J=6.2 Hz, 7H). LC-MS (M+H)⁺=449.0.

### Example 2: 7-(5-fluoro-2-(((3S,4R)-3-hydroxytetrahydro-2H-pyran-4-yl)amino)pyrimidin-4-yl)-1-isopropyl-3-methylquinolin-4(1H)-one

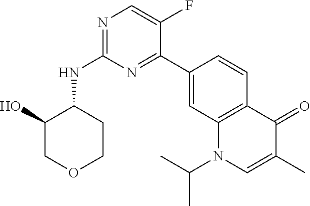 [SMILES: Cc1cn(C(C)C)c2cc(-c3nc(N[C@@H]4CCOC[C@H]4O)ncc3F)ccc2c1=O]

### Step 1: 7-bromo-1-isopropyl-3-methylquinolin-4(1H)-one

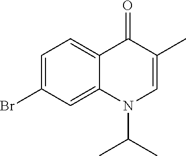 [SMILES: Cc1cn(C(C)C)c2cc(Br)ccc2c1=O]

To a solution of 7-bromo-3-methylquinolin-4(1H)-one (300 mg, 1.26 mmol) in dimethylformamide (5 mL) were added potassium carbonate (1.043 g, 7.56 mmol) and 2-iodopropane (1.071 g, 6.30 mmol). The resulting mixture was heated to 80° C. and stirred at 80° C. for 14 h before cooled to room temperature and concentrated. Water (5 mL) was added and the aqueous layer was extracted with ethyl acetate (30 mL×3). The combined organic layers were washed with brine, dried over sodium sulfate, filtered and concentrated. The residue was purified over silica gel by column chromatography to give the title compound (40 mg, 11%). LC-MS (M+H)⁺=280.0.

### Step 2: 1-isopropyl-3-methyl-7-(4,4,5,5-tetramethyl-1,3,2-dioxaborolan-2-yl)quinolin-4(1H)-one

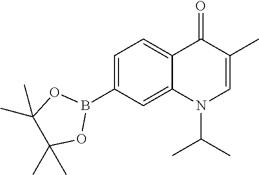

The title compound (42 mg, 89.9%) was prepared in a manner similar to that in Example 1 step 6 from 7-bromo-1-isopropyl-3-methylquinolin-4(1H)-one and bis(pinacolato)diboron. LC-MS (M+H)⁺=328.0.

### Step 3: 7-(2-chloro-5-fluoropyrimidin-4-yl)-1-isopropyl-3-methylquinolin-4(1H)-one

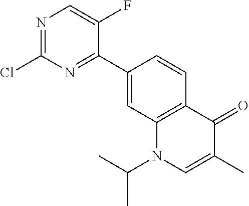 [SMILES: Cc1cn(C(C)C)c2cc(-c3nc(Cl)ncc3F)ccc2c1=O]

The title compound (26 mg, 61%) was prepared in a manner similar to that in Example 1 step 7 from 1-isopropyl-3-methyl-7-(4,4,5,5-tetramethyl-1,3,2-dioxaborolan-2-yl)quinolin-4(1H)-one and 2,4-dichloro-5-fluoropyrimidine. LC-MS (M+H)⁺=332.0.

### Step 4: 7-(5-fluoro-2-(((3S,4R)-3-hydroxytetrahydro-2H-pyran-4-yl)amino)pyrimidin-4-yl)-1-isopropyl-3-methylquinolin-4(1H)-one

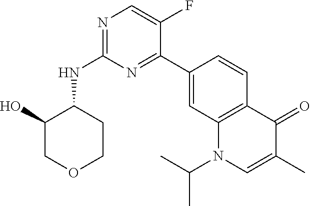 [SMILES: Cc1cn(C(C)C)c2cc(-c3nc(N[C@@H]4CCOC[C@H]4O)ncc3F)ccc2c1=O]

A mixture of 7-(2-chloro-5-fluoropyrimidin-4-yl)-1-isopropyl-3-methylquinolin-4(1H)-one (26 mg, 0.0784 mmol), (3S,4R)-4-aminotetrahydro-2H-pyran-3-ol hydrochloride (24 mg, 0.0157 mmol), tris(dibenzylideneacetone)dipalladium(0) (7.2 mg, 0.00784 mmol), 4,5-bis(diphenylphosphino)-9,9-dimethylxanthene (9.07 mg, 0.0157 mmol) and cesium carbonate (76.6 mg, 0.235 mmol) in 1,4-dioxane (5 mL) was stirred at 100° C. under nitrogen for 14 h before cooled to room temperature. Solvent was removed under reduced pressure. Water (5 mL) was added and the aqueous layer was extracted with ethyl acetate (20 mL×3). The combined organic layers were washed with brine, dried over sodium sulfate, filtered and concentrated. The residue was purified over silica gel by column chromatography to give the title compound (5 mg, 61%). ¹H-NMR (400 MHz, DMSO-d6) δ 8.50 (d, J=3.4 Hz, 1H), 8.40 (s, 1H), 8.35 (d, J=8.4 Hz, 1H), 8.16 (s, 1H), 7.86 (d, J=8.3 Hz, 1H), 7.29 (d. J=7.4 Hz, 1H), 5.11-4.99 (m, 1H), 4.94 (d, J=5.2 Hz, 1H), 3.89-3.75 (m, 3H), 3.54 (s, 1H), 3.36 (d, J=11.4 Hz, 1H), 3.06 (t, J=10.3 Hz, 1H), 2.05 (s, 3H), 2.08-1.90 (m, 1H), 1.52 (d, J=6.5 Hz, 6H), 1.58-1.45 (m, 1H). LC-MS (M+H)⁺=413.0.

### Example 3: 7-(5-fluoro-2-(((3S,4R)-3-hydroxytetrahydro-2H-pyran-4-yl)amino)pyrimidin-4-yl)-1-isopropylquinolin-4(1H)-one

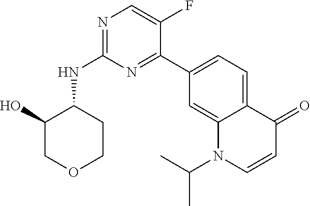 [SMILES: CC(C)n1ccc(=O)c2ccc(-c3nc(N[C@@H]4CCOC[C@H]4O)ncc3F)cc21]

### Step 1: ethyl (Z)-2-(4-bromo-2-fluorobenzoyl)-3-(dimethylamino)acrylate

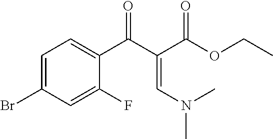 [SMILES: CCOC(=O)/C(=C\N(C)C)C(=O)c1ccc(Br)cc1F]

To a solution of ethyl (2E)-3-(dimethylamino)prop-2-enoate (1.2 mL, 8.36 mmol) and N,N-diisopropylethylamine (3.06 mL, 17.6 mmol) in toluene (10.0 mL) were added 4-bromo-2-fluorobenzoyl chloride (1.14 mL, 8.36 mmol). The resulting mixture was stirred for 3 h at 90° C. under nitrogen atmosphere. The solvent was removed under reduced pressure. The residue was purified by flash chromatography eluting with ethyl acetate in petroleum ether (0% to 50% gradient, v/v) to yield the title compound (2.5 g, 86%). LC-MS (M+H)⁺=343.9.

### Step 2: ethyl 7-bromo-1-isopropyl-4-oxo-1,4-dihydroquinoline-3-carboxylate

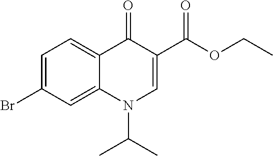 [SMILES: CCOC(=O)c1cn(C(C)C)c2cc(Br)ccc2c1=O]

To a solution of ethyl (Z)-2-(4-bromo-2-fluorobenzoyl)-3-(dimethylamino)acrylate (1.5 g, 4.36 mmol) in toluene (20 mL) was added isopropylamine (335 mg, 5.67 mmol). The resulting mixture was stirred for 2 h at 110° C. under nitrogen atmosphere. The resulting mixture was concentrated under vacuum. The residue was dissolved in dimethylformamide (20.0 mL) followed by addition of potassium carbonate (1.51 g, 10.9 mmol) at room temperature. The resulting mixture was stirred at 100° C. for 12 h under nitrogen atmosphere. The reaction was cooled to room temperature and quenched by addition of water (60 mL). The resulting mixture was extracted with dichloromethane (30 mL×3). The organic phases were combined, washed with brine and dried over sodium sulfate, filtered and concentrated. Theresidue was purified by flash chromatography eluting with ethyl acetate in petroleum ether (0% to 30% gradient) to yield the title compound (1.1 g, 74%). LC-MS (M+H)⁺=337.9.

### Step 3: 7-bromo-1-isopropyl-4-oxo-1,4-dihydroquinoline-3-carboxylic acid

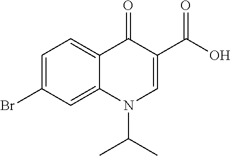 [SMILES: CC(C)n1cc(C(=O)O)c(=O)c2ccc(Br)cc21]

The title compound (930 mg, 92%) was prepared in a manner similar to Example 1 step 4 from ethyl 7-bromo-1-isopropyl-4-oxo-1,4-dihydroquinoline-3-carboxylate . LC-MS (M+H)⁺=310.0.

### Step 4: 7-bromo-1-isopropylquinolin-4(1H)-one

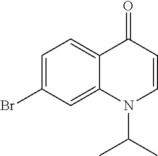

The title compound (550 mg, 72%) was prepared in a manner similar to Example 1 step 5 from 7-bromo-1-isopropyl-4-oxo-1,4-dihydroquinoline-3-carboxylic acid . LC-MS (M+H)⁺=265.9.

### Step 5: 1-isopropyl-7-(4,4,5,5-tetramethyl-1,3,2-dioxaborolan-2-yl)quinolin-4(1H)-one

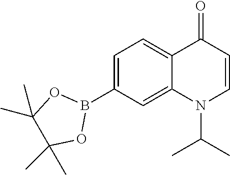 [SMILES: CC(C)n1ccc(=O)c2ccc(B3OC(C)(C)C(C)(C)O3)cc21]

The title compound (500 mg, 84%) was prepared in a manner similar to that in Example 1 step 6 from 7-bromo-1-isopropylquinolin-4(1H)-one and bis(pinacolato)diboron. LC-MS (M+H)+=314.1.

### Step 6: 7-(2-chloro-5-fluoropyrimidin-4-yl)-1-isopropylquinolin-4(1H)-one

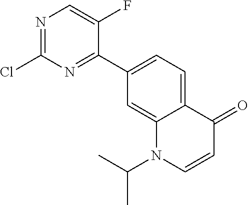 [SMILES: CC(C)n1ccc(=O)c2ccc(-c3nc(Cl)ncc3F)cc21]

The title compound (273 mg, 71%) was prepared in a manner similar to that in Example 1 step 7 from 1-isopropyl-7-(4,4,5,5-tetramethyl-1,3,2-dioxaborolan-2-yl)quinolin-4(1H)-one. LC-MS (M+H)⁺=318.0.

### Step 7: 7-(5-fluoro-2-(((3S,4R)-3-hydroxytetrahydro-2H-pyran-4-yl)amino)pyrimidin-4-yl)-1-isopropylquinolin-4(1H)-one

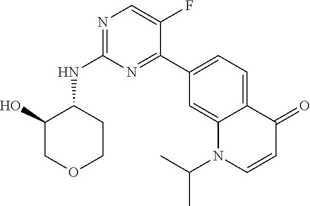 [SMILES: CC(C)n1ccc(=O)c2ccc(-c3nc(N[C@@H]4CCOC[C@H]4O)ncc3F)cc21]

The title compound (35 mg, 15%) was prepared in a manner similar to that in Example 1 step 8 from 7-(2-chloro-5-fluoropyrimidin-4-yl)-1-isopropylquinolin-4(1H)-one and (3S,4R)-4-aminooxan-3-ol hydrochloride. ¹H-NMR (300 MHz, DMSO-d6) δ 8.57-8.40 (m, 2H), 8.34 (d, J=8.4 Hz, 1H), 8.19 (d. J=7.9 Hz, 1H), 7.91 (d, J=8.4 Hz, 1H), 7.32 (d, J=7.7 Hz, 1H), 6.18 (d, J=7.9 Hz, 1H), 5.12-4.84 (m, 2H), 3.91-3.78 (m, 3H), 3.58-3.52 (m, 1H), 3.42-3.32 (m, 1H), 3.13-3.00 (m, 1H), 2.08-1.98 (m, 1H), 1.52 (d, J=6.6 Hz, 6H), 1.58-1.45 (m, 1H). LC-MS (M+H)⁺=399.1.

### Example 4: 3-acetyl-7-(5-fluoro-2-(((3S,4R)-3-hydroxytetrahydro-2H-pyran-4-yl)amino)pyrimidin-4-yl)-1-isopropylquinolin-4(1H)-one

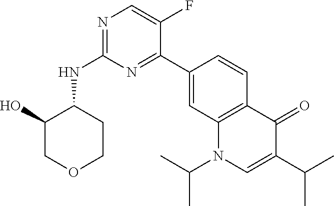 [SMILES: CC(C)c1cn(C(C)C)c2cc(-c3nc(N[C@@H]4CCOC[C@H]4O)ncc3F)ccc2c1=O]

### Step 1: 3-acetyl-7-bromo-1-isopropylquinolin-4(1H)-one

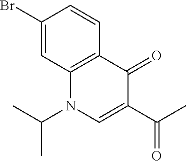 [SMILES: CC(=O)c1cn(C(C)C)c2cc(Br)ccc2c1=O]

To a solution of ethyl 7-bromo-1-isopropyl-4-oxo-1,4-dihydroquinoline-3-carboxylate (2.6 g, 7.7 mmol) in anhydrous tetrahydrofuran (100 mL) was added solution of 3 M methyl magnesium bromide solution in ethyl ether (3.1 mL, 9.3 mmol) dropwise at 0° C. The resulting solution was stirred at room temperature for 4 h before quenched by water (50 mL) and extracted with ethyl acetate (100 mL×3). The combined organic phases were dried over sodium sulfate, filtered and evaporated in vacuo. The residue was purified over silica gel by combi-flash, eluting with ethyl acetate in petroleum ether (33%, v/v) to give the title compound (0.47 g, 20%). LC-MS (M+H)⁺=308.1, 310.1.

### Step 2: 3-acetyl-1-isopropyl-7-(4,4,55-tetramethyl-1,3,2-dioxaborolan-2-yl)quinolin-4(1H)-one

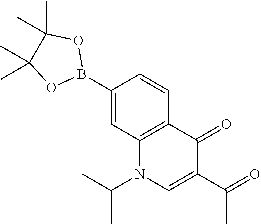 [SMILES: CC(=O)c1cn(C(C)C)c2cc(B3OC(C)(C)C(C)(C)O3)ccc2c1=O]

The title compound (110 mg, 30%) was prepared in a manner similar to Example 1 step 6 from 3-acetyl-7-bromo-1-isopropylquinolin-4(1H)-one and bis(pinacolato)diboron. LC-MS (M+H)⁺=356.0.

### Step 3: 3-acetyl-7-(2-chloro-5-fluoropyrimidin-4-yl)-1-isopropylquinolin-4(1H)-one

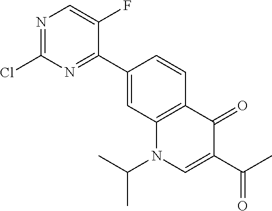 [SMILES: CC(=O)c1cn(C(C)C)c2cc(-c3nc(Cl)ncc3F)ccc2c1=O]

The title compound (70 mg, 65%) was prepared in a manner similar to Example 1 step 7 from 3-acetyl-1-isopropyl-7-(4,4,5,5-tetramethyl-1,3,2-dioxaborolan-2-yl)quinolin-4(1H)-one. LC-MS (M+H)⁺=360.1.

### Step 4: 3-acetyl-7-(5-fluoro-2-(((3S,4R)-3-hydroxytetrahydro-2H-pyran-4-yl)amino)pyrimidin-4-yl)-1-isopropylquinolin-4(1H)-one

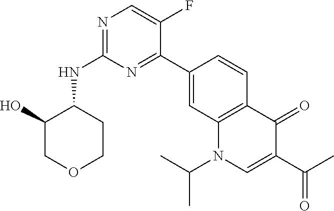 [SMILES: CC(=O)c1cn(C(C)C)c2cc(-c3nc(N[C@@H]4CCOC[C@H]4O)ncc3F)ccc2c1=O]

The title compound (5 mg, 6%) was prepared in a manner similar to Example 2 step 4 from 3-acetyl-7-(2-chloro-5-fluoropyrimidin-4-yl)-1-isopropylquinolin-4(1H)-one and (3S,4R)-4-aminotetrahydro-2H-pyran-3-ol hydrochloride. ¹H-NMR (400 MHz, DMSO-d6) δ 8.68 (s, 1H), 8.54 (d, J=3.3 Hz, 1H), 8.49 (d, J=8.4 Hz, 2H), 8.04 (d, J=8.2 Hz, 1H), 7.35 (d, J=7.7 Hz, 1H), 5.23-5.05 (m, 1H), 4.95 (d, J=5.3 Hz, 1H), 3.90-3.73 (m, 3H), 3.54 (s, 1H), 3.37 (s, 1H), 3.06 (t, J=10.4 Hz, 1H), 2.65 (s, 3H), 2.01 (s, 1H), 1.58 (d, J=6.5 Hz, 6H), 1.61-1.41 (m, 1H). LC-MS (M+H)⁺=441.1.

### Example 5: 7-(5-fluoro-2-(((3S,4R)-3-hydroxytetrahydro-2H-pyran-4-yl)amino)pyrimidin-4-yl)-3-(2-hydroxypropan-2-yl)-1-isopropylquinolin-4(1H)-one

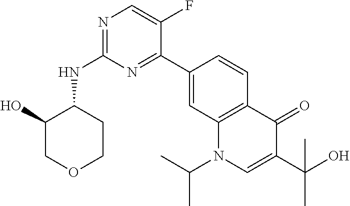 [SMILES: CC(C)n1cc(C(C)(C)O)c(=O)c2ccc(-c3nc(N[C@@H]4CCOC[C@H]4O)ncc3F)cc21]

### Step 1: 7-bromo-3-(2-hydroxypropan-2-yl)-1-isopropylquinolin-4(1H)-one

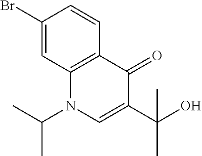 [SMILES: CC(C)n1cc(C(C)(C)O)c(=O)c2ccc(Br)cc21]

To a solution of ethyl 7-bromo-1-isopropyl-4-oxo-1,4-dihydroquinoline-3-carboxylate (2.6 g, 7.7 mmol) in anhydrous tetrahydrofuran (100 mL) was added solution of 3 M methyl magnesium bromide solution in ethyl ether (3.1 mL, 9.3 mmol) dropwise at 0° C. Then the resulting solution was stirred at room temperature for 4 h. The reaction was quenched by water (50 mL) and aqueous layer was extracted with ethyl acetate (100 mL×3). The combined organic phases were dried over sodium sulfate, filtered and evaporated in vacuo. The residue was purified over silica gel by combi-flash, eluting with ethyl acetate in petroleum ether (33%, v/v) to give the title compound (0.65 g, 28%). LC-MS (M+H)⁺=324.1, 326.1.

### Step 2: 3-(2-hydroxypropan-2-yl)-1-isopropyl-7-(4,4,5,5-tetramethyl-1,3,2-dioxaborolan-2-yl)quinolin-4(1H)-one

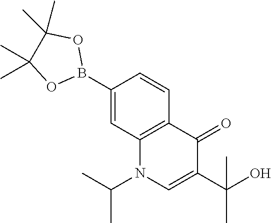 [SMILES: CC(C)n1cc(C(C)(C)O)c(=O)c2ccc(B3OC(C)(C)C(C)(C)O3)cc21]

The title compound (110 mg, 34%) was prepared in a manner similar to Example 1 step 6 from 7-bromo-3-(2-hydroxypropan-2-yl)-1-isopropylquinolin-4(1H)-one and bis(pinacolato)diboron. LC-MS (M+H)⁺=372.1.

### Step 3: 7-(2-chloro-5-fluoropyrimidin-4-yl)-3-(2-hydroxypropan-2-yl)-1-isopropylquinolin-4(1H)-one

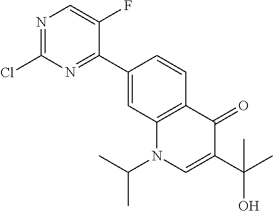 [SMILES: CC(C)n1cc(C(C)(C)O)c(=O)c2ccc(-c3nc(Cl)ncc3F)cc21]

The title compound (110 mg, 34%) was prepared in a manner similar to Example 1 step 7 from 3-(2-hydroxypropan-2-yl)-1-isopropyl-7-(4,4,5,5-tetramethyl-1,3,2-dioxaborolan-2-yl)quinolin-4(1H)-one and 2,4-dichloro-5-fluoropyrimidine. LC-MS (M+H)⁺=376.1.

### Step 4: 7-(5-fluoro-2-(((3S,4R)-3-hydroxytetrahydro-2H-pyran-4-yl)amino)pyrimidin-4-yl)-3-(2-hydroxypropan-2-yl)-1-isopropylquinolin-4(1H)-one

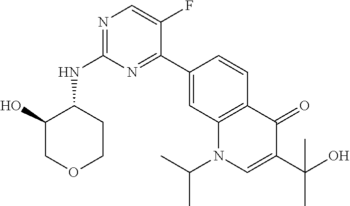 [SMILES: CC(C)n1cc(C(C)(C)O)c(=O)c2ccc(-c3nc(N[C@@H]4CCOC[C@H]4O)ncc3F)cc21]

The title compound (7 mg, 7%) was prepared in a manner similar to Example 2 step 4 from 7-(2-chloro-5-fluoropyrimidin-4-yl)-3-(2-hydroxypropan-2-yl)-1-isopropylquinolin-4(1H)-one and (3S,4R)-4-aminotetrahydro-2H-pyran-3-ol hydrochloride. ¹H-NMR (400 MHz, DMSO-d6) δ 8.51 (d, J=3.3 Hz, 1H), 8.42 (s, 1H), 8.38 (d, J=8.5 Hz, 1H), 8.23 (s, 1H), 7.89 (d, J=8.0 Hz, 1H), 7.33 (d, J=7.7 Hz, 1H), 5.44 (s, 1H), 5.14-5.02 (m, 1H), 4.95 (d, J=5.2 Hz, 1H), 3.90-3.71 (m, 3H), 3.53 (s, 1H), 3.40-3.25 (m, 1H), 3.06 (t, J=10.4 Hz, 1H), 2.01 (s, 1H), 1.63-1.43 (m, 13H). LC-MS (M+H)⁺=457.1.

### Example 6: 7-(5-fluoro-2-(((3S,4R)-3-hydroxytetrahydro-2H-pyran-4-yl)amino)pyrimidin-4-yl)-1-isopropyl-2-methylquinolin-4(1H)-one

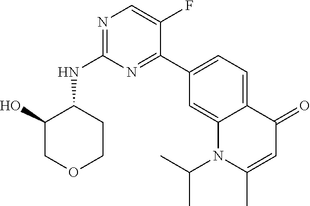 [SMILES: Cc1cc(=O)c2ccc(-c3nc(N[C@@H]4CCOC[C@H]4O)ncc3F)cc2n1C(C)C]

### Step 1: (E)-1-(4-bromo-2-fluorophenyl)-3-(dimethylamino)but-2-en-1-one

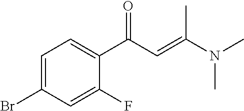 [SMILES: C/C(=C\C(=O)c1ccc(Br)cc1F)N(C)C]

A mixture of 1-(4-bromo-2-fluorophenyl)ethanone (7 g, 32.25 mmol) in (1,1-dimethoxyethyl)dimethylamine (14.00 g, 105 mmol) was stirred for 1 h at 120° C. under nitrogen atmosphere before cooled to room temperature. The solvent was removed under reduced pressure. The residue was purified by flash chromatography eluting with ethyl acetate in petroleum ether (0% to 10% gradient, v/v) to yield the title compound (7.2 g, 78%). LC-MS (M+H)⁺=286.0.

### Step 2: (E)-1-(4-bromo-2-fluorophenyl)-3-(isopropylamino)but-2-en-1-one

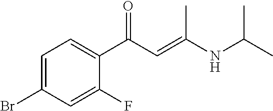 [SMILES: C/C(=C\C(=O)c1ccc(Br)cc1F)NC(C)C]

A mixture of (E)-1-(4-bromo-2-fluorophenyl)-3-(dimethylamino)but-2-en-1-one (7.2 g, 25.16 mmol) and isopropylamine (1.93 g, 32.71 mmol) in toluene (60 mL) was stirred for 14 h at 110° C. under nitrogen atmosphere before cooled to room temperature. The solvent was removed under reduced pressure. The residue was purified by flash chromatography eluting with ethyl acetate in petroleum ether (0% to 25% gradient, v/v) to yield the title compound (5.6 g, 74%). LC-MS (M+H)⁺=300.0.

### Step 3: 7-bromo-1-isopropyl-2-methylquinolin-4(1H)-one

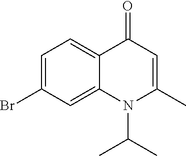 [SMILES: Cc1cc(=O)c2ccc(Br)cc2n1C(C)C]

A mixture of (E)-1-(4-bromo-2-fluorophenyl)-3-(isopropylamino)but-2-en-1-one (5.6 g, 18.66 mmol) and cesium carbonate (12.16 g, 37.31 mmol) in dimethylformamide (60 mL) was stirred for 14 h at 100° C. under nitrogen atmosphere before cooled to room temperature. The reaction was then quenched by the addition of water (50 mL). The resulting solution was extracted with ethyl acetate (40 mL×3). The organic phases were combined, washed with brine, dried over sodium sulfate, filtered and concentrated under reduced pressure. The residue was purified by flash chromatography eluting with ethyl acetate in petroleum ether (0% to 20% gradient, v/v) to yield the title compound (4 g, 76%). LC-MS (M+H)⁺=279.9.

### Step 4: 1-isoproyl-2-methyl-7-(4,4,5,5-tetramethyl-1,3,2-dioxaborolan-2-yl)quinolin-4(1H)-one

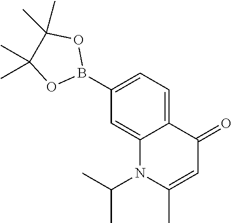 [SMILES: Cc1cc(=O)c2ccc(B3OC(C)(C)C(C)(C)O3)cc2n1C(C)C]

The title compound (200 mg, 34%) was prepared in a manner similar to that in Example 1 step 6 from 7-bromo-1-isopropyl-2-methylquinolin-4(1H)-one. LC-MS (M+H)⁺=328.2.

### Step 5: 7-(2-chloro-5-fluoropyrimidin-4-yl)-1-isopropyl-2-methylquinolin-4(1H)-one

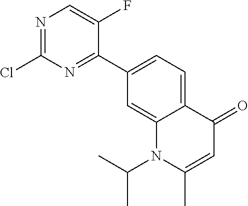 [SMILES: Cc1cc(=O)c2ccc(-c3nc(Cl)ncc3F)cc2n1C(C)C]

The title compound (50 mg, 25%) was prepared in a manner similar to that in Example 1 step 7 from 1-isopropyl-2-methyl-7-(4,4,5,5-tetramethyl-1,3,2-dioxaborolan-2-yl)quinolin-4(1H)-one and 2,4-dichloro-5-fluoropyrimidine. LC-MS (M+H)⁺=332.0.

### Step 6: 7-(5-fluoro-2-(((3S,4R)-3-hydroxytetrahydro-2H-pyran-4-yl)amino)pyrimidin-4-yl)-1-isopropyl-2-methylquinolin-4(1H)-one

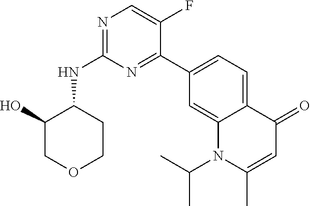 [SMILES: Cc1cc(=O)c2ccc(-c3nc(N[C@@H]4CCOC[C@H]4O)ncc3F)cc2n1C(C)C]

The title compound (12 mg, 10%) was prepared in a manner similar to Example 2 step 4 from 7-(2-chloro-5-fluoropyrimidin-4-yl)-1-isopropyl-2-methylquinolin-4(1H)-one and (3S,4R)-4-aminooxan-3-ol hydrochloride. ¹H-NMR (300 MHz, DMSO-d6) δ 8.65 (s, 1H), 8.51 (d, J=3.8 Hz, 1H), 8.29 (d, J=8.4 Hz, 1H), 7.92 (d, J=8.4 Hz, 1H), 7.30 (d, J=7.9 Hz, 1H), 6.12 (s, 1H), 5.17-5.04 (m, 1H), 4.97 (d, J=5.3 Hz, 1H), 3.98-3.77 (m, 3H), 3.62-3.46 (m, 1H), 3.40-3.27 (m, 1H), 3.11-2.98 (m, 1H), 2.56 (s, 3H), 2.11-1.97 (m, 1H), 1.76-1.59 (m, 6H), 1.57-1.48 (m, 1H). LC-MS (M+H)⁺=413.2.

### Example 7: 7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

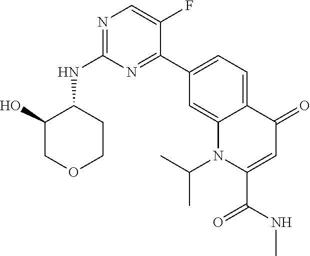 [SMILES: CNC(=O)c1cc(=O)c2ccc(-c3nc(N[C@@H]4CCOC[C@H]4O)ncc3F)cc2n1C(C)C]

### Step 1: 1-(4-bromo-2-(isopropylamino)phenyl)ethan-1-one

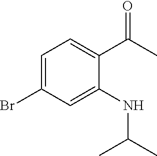

To a solution of 1-(4-bromo-2-fluorophenyl)ethanone (6 g, 27.645 mmol) and isopropylamine (2.45 g, 41.47 mmol) in dimethylacetamide (20 mL) were added potassium carbonate (5.73 g, 41.47 mmol). The resulting mixture was stirred at 130° C. for 3 h under nitrogen atmosphere. The reaction was then quenched by addition of water (200 mL). The resulting solution was extracted with ethyl acetate (100 mL×3). The combined organic phases were washed with brine, dried over sodium sulfate, filtered and concentrated under reduced pressure. The residue was purified by flash chromatography eluting with dichloromethane in petroleum ether (0% to 50% gradient, v/v) to yield the title compound (5.6 g, 79%). LC-MS (M+H)⁺=256.0.

### Step 2: ethyl 2-((2-acetyl-5-bromophenyl)(isopropyl)amino)-2-oxoacetate

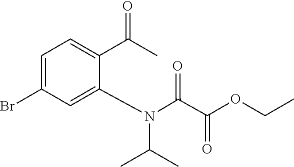 [SMILES: CCOC(=O)C(=O)N(c1cc(Br)ccc1C(C)=O)C(C)C]

To a solution of 1-(4-bromo-2-(isopropylamino)phenyl)ethan-1-one (5.6 g, 22.1 mmol) in tetrahydrofuran (100 mL) were added ethyl chloroglyoxylate (4.82 g, 35.3 mmol) and triethylamine (6.14 mL, 44.16 mmol) dropwise at 0° C. under nitrogen atmosphere. The resulting mixture was stirred at 25° C. for 1 h and 40° C. for another 1 h under nitrogen atmosphere before cooled to room temperature. The mixture was concentrated under reduced pressure and the residue was purified by flash chromatography eluting with ethyl acetate in petroleum ether (0% to 50% gradient, v/v) to yield the title compound (6.86 g, 87%). LC-MS (M+H)⁺=355.9.

### Step 3: ethyl 7-bromo-1-isopropyl-4-oxo-1,4-dihydroquinoline-2-carboxylate

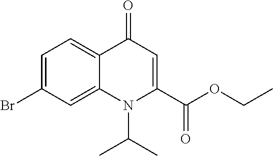 [SMILES: CCOC(=O)c1cc(=O)c2ccc(Br)cc2n1C(C)C]

To a solution of ethyl 2-((2-acetyl-5-bromophenyl)(isopropyl)amino)-2-oxoacetate (6.86 g, 19.3 mmol) in ethanol (100.0 mL) was added potassium carbonate (7.99 g, 57.8 mmol). The resulting mixture was stirred at 55° C. for 3 h under nitrogen atmosphere before cooled to room temperature. The resulting mixture was filtered and the filter cake was washed with ethyl acetate (100 mL×3). The filtrate was concentrated under reduced pressure and the residue was purified by flash chromatography eluting with ethyl acetate in petroleum ether (0% to 100% gradient, v/v) to yield the title compound (3.8 g, 58%). LC-MS (M+H)⁺=337.9.

### Step 4: 7-bromo-1-isopropyl-4-oxo-1,4-dihydroquinoline-2-carboxylic acid

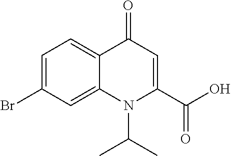 [SMILES: CC(C)n1c(C(=O)O)cc(=O)c2ccc(Br)cc21]

The title compound (110 mg, 41%) was prepared in a manner similar to Example 1 step 4 from ethyl 7-bromo-1-isopropyl-4-oxo-1,4-dihydroquinoline-2-carboxylate. LC-MS (M+H)⁺=310.0.

### Step 5: 7-bromo-1-isopropyl-N-methyl-4-oxo-1,4-dihydroquinoline-2-carboxamide

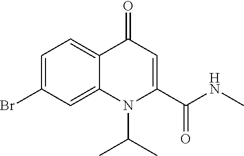 [SMILES: CNC(=O)c1cc(=O)c2ccc(Br)cc2n1C(C)C]

To a mixture of 7-bromo-1-isopropyl-4-oxo-1,4-dihydroquinoline-2-carboxylic acid (110 mg, 0.36 mmol), N,N-diisopropylethylamine (138 mg, 1.06 mmol) and O-(7-azabenzotriazol-1-yl)-N,N,N′,N′-tetramethyluronium hexafluorophosphate (203 mg, 0.53 mmol) in dimethylformamide (2 mL) was added methylamine (0.43 mL, 0.430 mmol, 1M in tetrahydrofuran) dropwise at room temperature. The resulting mixture was stirred at room temperature for 14 h under nitrogen atmosphere before quenched by addition of water (10 mL). The resulting solution was extracted with ethyl acetate (15 mL×3). The organic phases were combined, washed with brine and dried over sodium sulfate, filtered and concentrated under reduced pressure. The residue was purified by flash chromatography eluting with dichloromethane in petroleum ether (0% to 50% gradient, v/v) to yield the title compound (85 mg, 74%). LC-MS (M+H)⁺=322.9.

### Step 6: (1-isopropyl-2-(methylcarbamoyl)-4-oxo-1,4-dihydroquinolin-7-yl)boronic acid

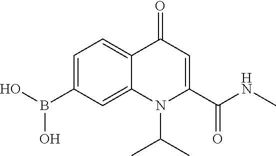 [SMILES: CNC(=O)c1cc(=O)c2ccc(B(O)O)cc2n1C(C)C]

The title compound (45 mg, 48%) was prepared in a manner similar to that in Example 1 step 6 from 7-bromo-1-isopropyl-N-methyl-4-oxo-1,4-dihydroquinoline-2-carboxamide. LC-MS (M+H)⁺=289.2.

### Step 7: 7-(2-chloro-5-fluoropyrimidin-4-yl)-1-isopropyl-N-methyl-4-oxo-1,4-dihydroquinoline-2-carboxamide

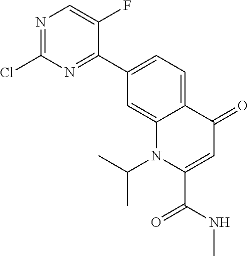 [SMILES: CNC(=O)c1cc(=O)c2ccc(-c3nc(Cl)ncc3F)cc2n1C(C)C]

The title compound (30 mg, 46%) was prepared in a manner similar to that in Example 1 step 7 from (1-isopropyl-2-(methylcarbamoyl)-4-oxo-1,4-dihydroquinolin-7-yl)boronic acid and 2,4-dichloro-5-fluoropyrimidine. LC-MS (M+H)⁺=375.1.

### Step 8: 7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

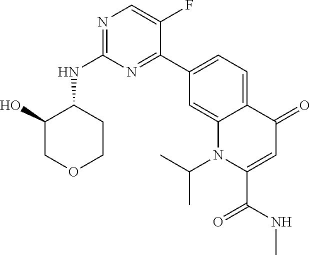 [SMILES: CNC(=O)c1cc(=O)c2ccc(-c3nc(N[C@@H]4CCOC[C@H]4O)ncc3F)cc2n1C(C)C]

The title compound (I mg, 4%) was prepared in a manner similar to that in Example 2 step 4 from 7-(2-chloro-5-fluoropyrimidin-4-yl)-1-isopropyl-N-methyl-4-oxo-1,4-dihydroquinoline-2-carboxamide and (3S,4R)-4-aminooxan-3-ol hydrochloride. ¹H-NMR (300 MHz, DMSO-d6) δ 9.03 (s, 1H), 8.53 (d, J=3.8 Hz, 1H), 8.32 (d, J=8.5 Hz, 1H), 7.98 (d, J=8.4 Hz, 1H), 7.33 (d, J=7.9 Hz, 1H), 6.08 (s, 1H), 4.97 (d, J=5.1 Hz, 11H), 4.79-4.64 (m, 1H), 3.90-3.79 (m, 3H), 3.61-3.45 (m, 1H), 3.29 (s, 1H), 3.10-2.97 (m, 1H), 2.82 (d, J=4.6 Hz, 3H), 2.06-1.96 (m, 1H), 1.76-1.67 (m, 6H), 1.56-1.44 (m, 1H). LC-MS (M+H)⁺=456.2.

### Example 8: 7-(5-fluoro-2-(((3S,4R)-3-hydroxytetrahydro-2H-pyran-4-yl)amino)pyrimidin-4-yl)-1-isopropyl-3-methoxyquinolin-4(11H)-one

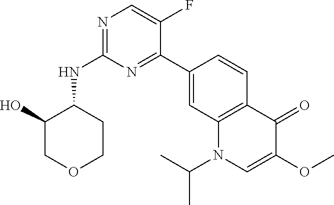 [SMILES: COc1cn(C(C)C)c2cc(-c3nc(N[C@@H]4CCOC[C@H]4O)ncc3F)ccc2c1=O]

### Step 1: 7-bromo-1-isopropyl-4-oxo-1,4-dihydroquinolin-3-yl acetate

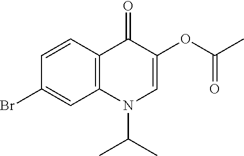 [SMILES: CC(=O)Oc1cn(C(C)C)c2cc(Br)ccc2c1=O]

To a solution of 3-acetyl-7-bromo-1-isopropylquinolin-4(1H)-one (440 mg, 1.42 mmol) in anhydrous dichloromethane (30 mL) was added 3-chloroperoxybenzoic acid (246 mg, 1.42 mmol) slowly at 0° C. The resulting solution was stirred at room temperature for 16 h. The reaction was quenched by water (20 mL) and aqueous layer was extracted with dichloromethane (30 mL×3). The combined organic phases were dried over sodium sulfate, filtered and evaporated in vacuo. The residue was purified over silica gel by combi-flash, eluting with ethyl acetate in petroleum ether (33%, v/v) to give the title compound (350 mg, 76%). LC-MS (M+H)⁺=324.1, 326.1.

### Step 2: 7-bromo-3-hydroxy-1-isopropylquinolin-4(1H)-one

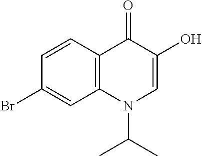 [SMILES: CC(C)n1cc(O)c(=O)c2ccc(Br)cc21]

A mixture of 7-bromo-1-isopropyl-4-oxo-1,4-dihydroquinolin-3-yl acetate (350 mg, 1.08 mmol) and potassium carbonate (298 mg, 2.16 mmol) in ethanol (20 mL) was stirred at 60° C. for 1 h before cooled to room temperature. The reaction mixture was filtered and the filtrate was concentrated under reduced pressure. The residue was purified over silica gel by combi-flash, eluting with ethyl acetate in petroleum ether (50%, v/v) to give the title compound (240 mg, 79%). LC-MS (M+H)⁺=282.1, 284.1.

### Step 3: 7-bromo-1-isopropyl-3-methoxyquinolin-4(1H)-one

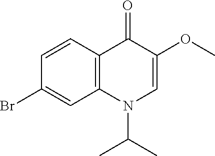 [SMILES: COc1cn(C(C)C)c2cc(Br)ccc2c1=O]

A mixture of 7-bromo-3-hydroxy-1-isopropylquinolin-4(1H)-one (120 mg, 0.43 mmol), iodomethane (121 mg, 0.85 mmol) and potassium carbonate (117 mg, 0.85 mmol) in dimethylformamide (10 mL) was stirred at room temperature for 1 h. Water (20 mL) was added and the aqueous layer was extracted with ethyl acetate (30 mL×3). The combined organic layers were dried over sodium sulfate, filtered and concentrated under reduced pressure. The residue was purified over silica gel by combi-flash, eluting with ethyl acetate in petroleum ether (50%, v/v) to give the title compound (110 mg, 87%). LC-MS (M+H)⁺=296.1, 298.1.

### Step 4: 1-isopropyl-3-methoxy-7-(4,4,5,5-tetramethyl-1,3,2-dioxaborolan-2-yl)quinolin-4(1H)-one

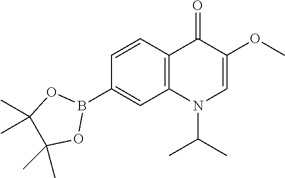

The title compound (91 mg, 71%) was prepared in a manner similar to Example 1 step 6 from 7-bromo-1-isopropyl-3-methoxyquinolin-4(1H)-one and bis(pinacolato)diboron. LC-MS (M+H)⁺=344.1.

### Step 5: 7-(2-chloro-5-fluoropyrimidin-4-yl)-1-isopropyl-3-methoxyquinolin-4(1H)-one

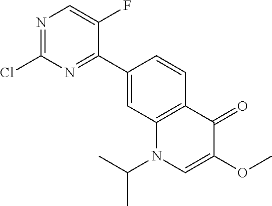 [SMILES: COc1cn(C(C)C)c2cc(-c3nc(Cl)ncc3F)ccc2c1=O]

The title compound (60 mg, 50%) was prepared in a manner similar to Example 1 step 7 from 1-isopropyl-3-methoxy-7-(4,4,5,5-tetramethyl-1,3,2-dioxaborolan-2-yl)quinolin-4(1H)-one and 2,4-dichloro-5-fluoropyrimidine. LC-MS (M+H)⁺=348.1.

### Step 5: 7-(5-fluoro-2-(((3S,4R)-3-hydroxytetrahydro-2H-pyran-4-yl)amino)pyrimidin-4-yl)-1-isopropyl-3-methoxyquinolin-4(1H)-one

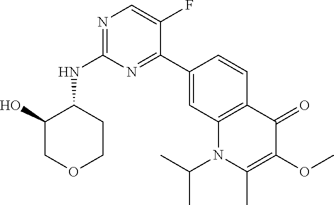 [SMILES: COc1c(C)n(C(C)C)c2cc(-c3nc(N[C@@H]4CCOC[C@H]4O)ncc3F)ccc2c1=O]

The title compound (12 mg, 16%) was prepared in a manner similar to Example 2 step 4 from 7-(2-chloro-5-fluoropyrimidin-4-yl)-1-isopropyl-3-methoxyquinolin-4(1H)-one and (3S,4R)-4-aminotetrahydro-2H-pyran-3-ol hydrochloride. ¹H-NMR (400 MHz, DMSO-d6) δ 8.51 (d, J=3.2 Hz, 11H), 8.43 (s, 1H), 8.39 (d, J=8.5 Hz, 1H), 8.00 (s, 1H), 7.84 (d, J=8.4 Hz, 1H), 7.31 (d, J=7.5 Hz, 1H), 5.19-5.03 (m, 1H), 4.95 (d, J=5.1 Hz, 1H), 3.83 (s, 3H), 3.92-3.72 (m, 3H), 3.54 (s, 1H), 3.37-3.35 (m, 1H), 3.06 (t, J=10.4 Hz, 1H), 2.00 (brs, 1H), 1.53 (t, J=10.8 Hz, 7H). LC-MS (M+H)⁺=429.1.

### Example 9: 3-cyclopropyl-7-(5-fluoro-2-(((3S,4R)-3-hydroxytetrahydro-2H-pyran-4-yl)amino)pyrimidin-4-yl)-1-isopropylquinolin-4(1H)-one

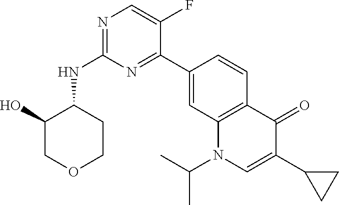 [SMILES: CC(C)n1cc(C2CC2)c(=O)c2ccc(-c3nc(N[C@@H]4CCOC[C@H]4O)ncc3F)cc21]

### Step 1: 3-bromo-7-(5-fluoro-2-(((3S,4R)-3-hydroxytetrahydro-2H-pyran-4-vi)amino)pyrimidin-4-yl)-1-isopropylquinolin-4(1H)-one

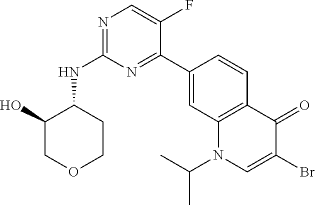 [SMILES: CC(C)n1cc(Br)c(=O)c2ccc(-c3nc(N[C@@H]4CCOC[C@H]4O)ncc3F)cc21]

To a mixture of 7-(5-fluoro-2-(((3S,4R)-3-hydroxytetrahydro-2H-pyran-4-yl)amino)pyrimidin-4-yl)-1-isopropylquinolin-4(1H)-one (100 mg, 0.25 mmol) in acetic acid (1 mL) was added N-bromosuccinimide (45 mg, 0.25 mmol). The mixture was stirred at room temperature for 14 h before diluted with saturated aqueous sodium bicarbonate solution (10 mL). The aqueous layer was extracted with ethyl acetate (3×10 mL). The combined organic layers were washed with brine (20 mL). dried over sodium sulfate, filtered and concentrated under vacuum. The residue was purified by preparative TLC eluting with methanol in dichloromethane (5%, v/v) to give the title compound (100 mg, 83%). LC-MS (M+H)⁺=477.3, 479.1.

### Step 2: 3-cyclopropyl-7-(5-fluoro-2-(((3S,4R)-3-hydroxytetrahydro-2H-pyran-4-yl)amino)pyrimidin-4-yl)-1-isopropylquinolin-4(1H)-one

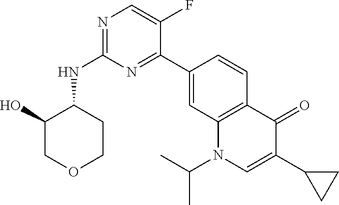 [SMILES: CC(C)n1cc(C2CC2)c(=O)c2ccc(-c3nc(N[C@@H]4CCOC[C@H]4O)ncc3F)cc21]

To a mixture of 3-bromo-7-(5-fluoro-2-(((3S,4R)-3-hydroxytetrahydro-2H-pyran-4-yl)amino)pyrimidin-4-yl)-1-isopropylquinolin-4(1H)-one (8 mg, 0.02 mmol) and cyclopropylboronic acid (10 mg, 0.11 mmol) in toluene (5 mL) and water (1 mL) was added potassium phosphate tribasic (30 mg, 0.14 mmol) and dichlorobis(tricyclohexylphosphine)palladium(II) (10 mg, 0.01 mmol). The mixture was stirred at 100° C. for 2 h. The mixture was cooled to room temperature and diluted with water (10 ml). The aqueous layer was extracted with ethyl acetate (3×10 mL). The combined organic layers were washed with brine (20 mL), dried over sodium sulfate, filtered and concentrated under vacuum. The residue was purified by preparative TLC eluting with methanol in dichloromethane (5%, v/v) to give the title compound (4 mg, 45%). ¹H-NMR (400 MHz, DMSO-d6) δ 8.50 (d, J=3.2 Hz, 1H), 8.46-8.31 (m, 2H), 7.87 (d, J=8.4 Hz, 1H), 7.74 (s, 1H), 7.32 (d. J=7.5 Hz, 1H), 5.08-4.98 (m, 1H), 4.96 (d, J=4.9 Hz, 1H), 3.88-3.75 (m, 3H), 3.59-3.48 (m, 1H), 3.39-3.29 (m, 1H), 3.10-3.03 (m, 1H), 2.08-1.91 (m, 2H), 1.57-1.45 (m, 7H), 0.84-0.71 (m, 4H). LC-MS (M+H)⁺=439.5.

### Example 10: 1-cyclopentyl-7-(5-fluoro-2-(((3S,4R)-3-hydroxytetrahydro-2H-pyran-4-yl)amino)pyrimidin-4-yl)-2-methylquinolin-4(1H)-one

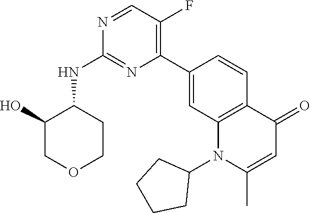 [SMILES: Cc1cc(=O)c2ccc(-c3nc(N[C@@H]4CCOC[C@H]4O)ncc3F)cc2n1C1CCCC1]

### Step 1: (E)-1-(4-bromo-2-fluorophenyl)-3-(cyclopentylamino)but-2-en-1-one

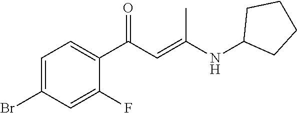 [SMILES: C/C(=C\C(=O)c1ccc(Br)cc1F)NC1CCCC1]

To a solution of (E)-1-(4-bromo-2-fluorophenyl)-3-(dimethylamino)but-2-en-1-one (1.5 g, 5.3 mmol) in toluene (20 mL) was added cyclopentanamine (536 mg, 6.3 mmol). The reaction mixture was stirred at 120° C. for 4 h. The reaction mixture was cooled to room temperature, and concentrated under reduced pressure. The crude (1.5 g, 88%) was used for the next step without further purification. LC-MS (M+H)⁺=326.1

### Step 2: 7-bromo-1-cyclopentyl-2-methylquinolin-4(1H)-one

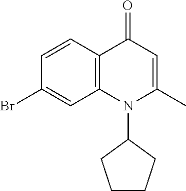 [SMILES: Cc1cc(=O)c2ccc(Br)cc2n1C1CCCC1]

To a solution of (E)-1-(4-bromo-2-fluorophenyl)-3-(cyclopentylamino)but-2-en-1-one (1.5 g, 4.6 mmol) in dimethylformamide (15 mL) was added cesium carbonate (3.0 g, 9.2 mmol). The mixture was stirred at 100° C. for overnight. The reaction mixture was cooled to room temperature and diluted with ethyl acetate (30 mL), washed with brine (30 mL), dried with over sodium sulfate, filtered and concentrated under reduced pressure. The residue was purified over silica gel by combi-flash, eluting with ethyl acetate in petroleum ether (50%, v/v) to give the title compound (1.1 g, 76%). LC-MS (M+H) *=306.1.

### Step 3: 1-cyclopentyl-2-methyl-7-(4,4,5,5-tetramethyl-1,3,2-dioxaborolan-2-yl)quinolin-4(1H)-one

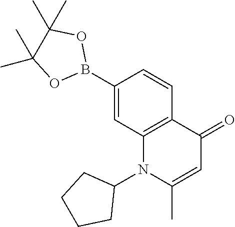 [SMILES: Cc1cc(=O)c2ccc(B3OC(C)(C)C(C)(C)O3)cc2n1C1CCCC1]

The title compound (358 mg, 100%) was prepared in a manner similar to Example 1 step 6 from 7-bromo-1-cyclopentyl-2-methylquinolin-4(1H)-one and and bis(pinacolato)diboron. LC-MS (M+H)⁺=354.0.

### Step 4: 7-(2-chloro-5-fluoropyrimidin-4-yl)-1-cyclopentyl-2-methylquinolin-4(1H)-one

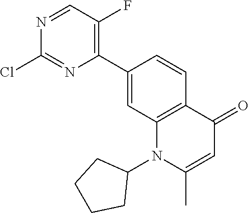 [SMILES: Cc1cc(=O)c2ccc(-c3nc(Cl)ncc3F)cc2n1C1CCCC1]

The title compound (362.3 mg, 100%) was prepared in a manner similar to Example 1 step 7 from 2,4-dichloro-5-fluoropyrimidine and 1-cyclopentyl-2-methyl-7-(4,4,5,5-tetramethyl-1,3,2-dioxaborolan-2-yl)quinolin-4(1H)-one. LC-MS (M+H)⁺=358.0.

### Step 5: 1: 1-cyclopentyl-7-(5-fluoro-2-(((3S,4R)-3-hydroxytetrahydro-2H-pyran-4-yl)amino)pyrimidin-4-yl)-2-methylquinolin-4(1H)-one

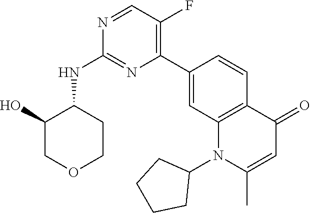 [SMILES: Cc1cc(=O)c2ccc(-c3nc(N[C@@H]4CCOC[C@H]4O)ncc3F)cc2n1C1CCCC1]

The title compound (65 mg, 22%) was prepared in a manner similar to Example 2 step 4 from 7-(2-chloro-5-fluoropyrimidin-4-yl)-1-cyclopentyl-2-methylquinolin-4(1H)-one and (3S,4R)-4-aminotetrahydro-2H-pyran-3-ol hydrochloride. ¹H-NMR (400 MHz, DMSO-d6) δ 8.51 (d, J=3.1 Hz, 1H), 8.31 (d, J=8.4 Hz, 1H), 8.19 (s, 1H), 7.86 (d, J=8.3 Hz, 1H), 7.25 (d, J=8.1 Hz, 1H), 6.14 (s, 1H), 5.25-5.12 (m, 1H), 4.95 (d, J=5.0 Hz, 1H), 3.83 (d, J=5.1 Hz, 3H), 3.59-3.45 (m, 1H), 3.35 (s, 1H), 3.04 (t, J=10.4 Hz, 1H), 2.58 (s, 3H), 2.25 (s, 2H), 2.10 (d, J=17.5 Hz, 4H), 1.97 (d, J=10.7 Hz, 1H), 1.80 (s, 2H), 1.50 (d, J=9.3 Hz, 1H). LC-MS (M+H)⁺=439.0.

### Example 11: 7-(5-chloro-2-(((3S,4R)-3-hydroxytetrahydro-2H-pyran-4-yl)amino)pyrimidin-4-yl)-1-isopropyl-2-methylquinolin-4(1H)-one

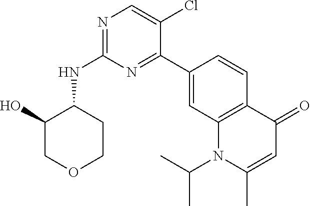 [SMILES: Cc1cc(=O)c2ccc(-c3nc(N[C@@H]4CCOC[C@H]4O)ncc3Cl)cc2n1C(C)C]

### Step 1: 7-(2,5-dichloropyrimidin-4-yl)-1-isopropyl-2-methylquinolin-4(1H)-one

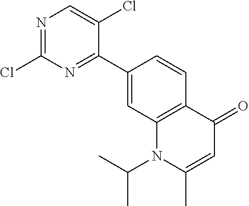 [SMILES: Cc1cc(=O)c2ccc(-c3nc(Cl)ncc3Cl)cc2n1C(C)C]

The title compound (313 mg, 99%) was prepared in a manner similar to Example 1 step 7 from 2,4,5-trichloropyrimidine and 1-isopropyl-2-methyl-7-(4,4,5,5-tetramethyl-1,3,2-dioxaborolan-2-yl)quinolin-4(1H)-one. LC-MS (M+H)⁺=348.

### Step 2: 7-(5-chloro-2-(((3S,4R)-3-hydroxytetrahydro-2H-pyran-4-yl)amino)pyrimidin-4-yl)-1-isopropyl-2-methylquinolin-4(1H)-one

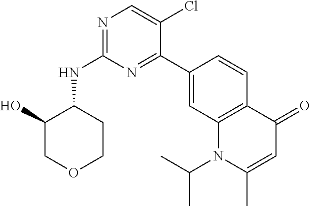 [SMILES: Cc1cc(=O)c2ccc(-c3nc(N[C@@H]4CCOC[C@H]4O)ncc3Cl)cc2n1C(C)C]

The title compound (19 mg, 29%) was prepared in a manner similar to Example 1 step 8 from 7-(2,5-dichloropyrimidin-4-yl)-1-isopropyl-2-methylquinolin-4(1H)-one and (3S,4R)-4-aminotetrahydro-2H-pyran-3-ol. ¹H-NMR (400 MHz, DMSO-d6) δ: 8.46 (s, 1H), 8.41-8.08 (m, 2H), 7.78-7.55 (m, 2H), 6.11 (s, 1H), 5.15-5.01 (m, 1H), 4.96 (d, J=3.8 Hz, 1H), 3.94-3.74 (m, 3H), 3.60-3.44 (m, 1H), 3.30-3.23 (m, 1H), 3.10-2.92 (m, 1H), 2.55 (s, 3H), 2.02-1.90 (d, J=9.9 Hz, 1H), 1.67 (d, J=6.8 Hz, 6H), 1.56-1.48 (m, 1H). LC-MS (M+H)⁺=429.

### Example 12: 3-chloro-7-(5-fluoro-2-(((3S,4R)-3-hydroxytetrahydro-2H-pyran-4-yl)amino)pyrimidin-4-yl)-1-isopropylquinolin-4(1H)-one

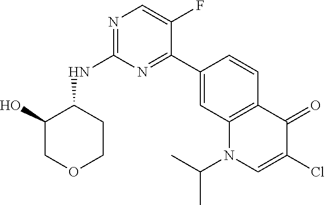 [SMILES: CC(C)n1cc(Cl)c(=O)c2ccc(-c3nc(N[C@@H]4CCOC[C@H]4O)ncc3F)cc21]

The title compound (30 mg, 69%) was prepared in a manner similar to Example 9 step 1 from 7-(5-fluoro-2-(((3S,4R)-3-hydroxytetrahydro-2H-pyran-4-yl)amino)pyrimidin-4-yl)-1-isopropylquinolin-4(1H)-one and N-chlorosuccinimide. ¹H-NMR (400 MHz, DMSO-d6) δ 8.59 (s, 1H), 8.52 (d, J=3.2 Hz, 1H), 8.48 (brs, 1H), 8.40 (d, J=8.5 Hz, 1H), 7.97 (d, J=8.4 Hz, 1H), 7.34 (d. J=7.7 Hz, 1H), 5.17-5.06 (m, 1H), 4.95 (d, J=5.3 Hz, 1H), 3.89-3.76 (m, 3H), 3.59-3.48 (m, 1H), 3.39-3.30 (m, 1H), 3.10-3.01 (m, 1H), 2.07-1.96 (m, 1H), 1.60-1.44 (m, 7H). LC-MS (M+H)⁺=433.5.

### Example 13: 7-(5-chloro-2-(((3S,4R)-3-hydroxytetrahydro-2H-pyran-4-yl)amino)pyrimidin-4-yl)-1-isopropyl-2-(morpholine-4-carbonyl)quinolin-4(1H)-one

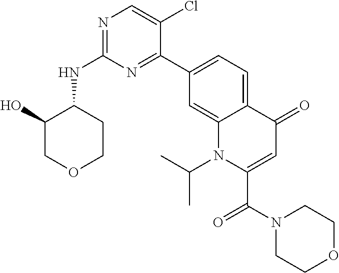 [SMILES: CC(C)n1c(C(=O)N2CCOCC2)cc(=O)c2ccc(-c3nc(N[C@@H]4CCOC[C@H]4O)ncc3Cl)cc21]

### Step 1: ethyl 1-isopropyl-4-oxo-7-(4,4,5,5-tetramethyl-1,3,2-dioxaborolan-2-yl)-1,4-dihydroquinoline-2-carboxylate

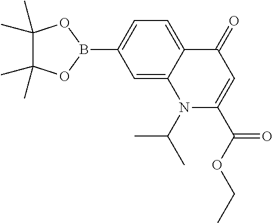 [SMILES: CCOC(=O)c1cc(=O)c2ccc(B3OC(C)(C)C(C)(C)O3)cc2n1C(C)C]

The title compound (4 g, 92%) was prepared in a manner similar to that in Example 1 step 6 from ethyl 7-bromo-1-isopropyl-4-oxo-1,4-dihydroquinoline-2-carboxylate and bis(pinacolato)diboron. LC-MS (M+H)⁺=386.1.

### Step 2: ethyl 7-(2,5-dichloropyrimidin-4-yl)-1-isopropyl-4-oxo-1,4-dihydroquinoline-2-carboxylate

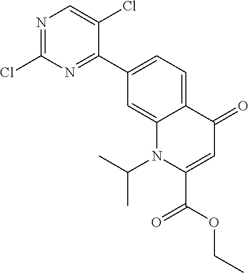 [SMILES: CCOC(=O)c1cc(=O)c2ccc(-c3nc(Cl)ncc3Cl)cc2n1C(C)C]

The title compound (1.5 g, 51%) was prepared in a manner similar to that in Example 1 step 7 from ethyl 1-isopropyl-4-oxo-7-(4,4,5,5-tetramethyl-1,3,2-dioxaborolan-2-yl)-1,4-dihydroquinoline-2-carboxylate and 2,4,5-trichloropyrimidine. LC-MS (M+H)⁺=406.0.

### Step 3: ethyl 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylate

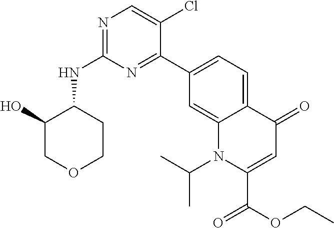 [SMILES: CCOC(=O)c1cc(=O)c2ccc(-c3nc(N[C@@H]4CCOC[C@H]4O)ncc3Cl)cc2n1C(C)C]

The title compound (900 mg, 50%) was prepared in a manner similar to that in Example 2 step 4 from ethyl 7-(2,5-dichloropyrimidin-4-yl)-1-isopropyl-4-oxo-1,4-dihydroquinoline-2-carboxylate and (3S,4R)-4-aminooxan-3-ol hydrochloride. LC-MS (M+H)⁺=487.1.

### Step 4: 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid

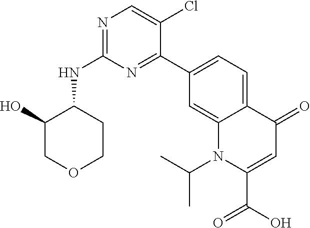 [SMILES: CC(C)n1c(C(=O)O)cc(=O)c2ccc(-c3nc(N[C@@H]4CCOC[C@H]4O)ncc3Cl)cc21]

The title compound (800 mg. 95%) was prepared in a manner similar to that in Example 1 step 4 from ethyl 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylate. LC-MS (M+H)⁺=459.1.

### Step 5: 7-(5-chloro-2-(((3S,4R)-3-hydroxytetrahydro-2H-pyran-4-yl)amino)pyrimidin-4-yl)-1-isopropyl-2-(morpholine-4-carbonyl)quinolin-4(11H)-one

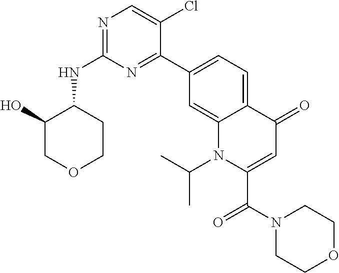 [SMILES: CC(C)n1c(C(=O)N2CCOCC2)cc(=O)c2ccc(-c3nc(N[C@@H]4CCOC[C@H]4O)ncc3Cl)cc21]

The title compound (30 mg, 51%) was prepared in a manner similar to that described in Example 7 step 5 from 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid and morpholine. ¹H-NMR (400 MHz, DMSO-d6) δ 8.47 (s, 1H), 8.37-8.17 (m, 2H), 7.92-7.64 (m, 1H), 7.58 (d, J=7.7 Hz, 1H), 6.11 (s, 1H), 4.96 (d, J=4.5 Hz, 1H), 4.69-4.57 (m, 1H), 3.95-3.56 (m, 8H), 3.48-3.40 (m, 4H), 3.31-3.18 (m, 1H), 3.07-2.94 (m, 1H), 2.01-1.90 (m, 1H), 1.80-1.62 (m, 6H), 1.58-1.45 (m, 1H). LC-MS (M+H)⁺=528.5.

### Example 14: 7-(5-chloro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

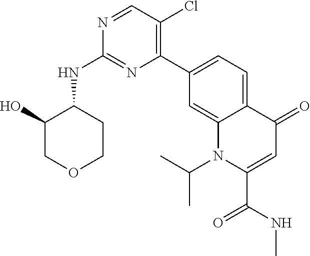 [SMILES: CNC(=O)c1cc(=O)c2ccc(-c3nc(N[C@@H]4CCOC[C@H]4O)ncc3Cl)cc2n1C(C)C]

### Step 1: 7-(2,5-dichloropyrimidin-4-yl)-1-isopropyl-N-methyl-4-oxo-1,4-dihydroquinoline-2-carboxamide

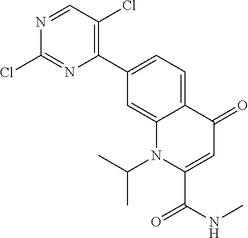 [SMILES: CNC(=O)c1cc(=O)c2ccc(-c3nc(Cl)ncc3Cl)cc2n1C(C)C]

The title compound (80 mg, 38%) was prepared in a manner similar to Example 1 step 7 from (1-isopropyl-2-(methylcarbamoyl)-4-oxo-1,4-dihydroquinolin-7-yl)boronic acid and 2,4,5-trichloropyrimidine. LC-MS (M+H)⁺=391.1.

### Step 2: 7-(5-chloro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

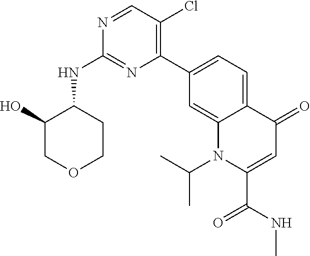 [SMILES: CNC(=O)c1cc(=O)c2ccc(-c3nc(N[C@@H]4CCOC[C@H]4O)ncc3Cl)cc2n1C(C)C]

The title compound (6 mg, 6%) was prepared in a manner similar to Example 1 step 8 from 7-(2,5-dichloropyrimidin-4-yl)-1-isopropyl-N-methyl-4-oxo-1,4-dihydroquinoline-2-carboxamide and (3S,4R)-4-aminotetrahydro-2H-pyran-3-ol hydrochloride. ¹H-NMR (400 MHz, DMSO-d6) δ 9.02 (s, 1H), 8.47 (s, 1H), 8.31-8.28 (m, 2H), 7.83 (brs, 1H), 7.57 (d, J=7.5 Hz, 1H), 6.08 (s, 1H), 4.95 (d, J=5.1 Hz, 1H), 4.82-4.62 (m, 1H), 3.82 (d, J=5.5 Hz, 3H), 3.51 (s, 1H), 3.32-3.23 (m, 1H), 3.01 (s, 1H), 2.81 (d, J=4.5 Hz, 3H), 1.94 (s, 1H), 1.67 (d, J=6.9 Hz, 6H), 1.52 (s, 1H). LC-MS (M+H)⁺=472.1.

### Example 15: 3-chloro-7-(5-fluoro-2-(((3S,4R)-3-hydroxytetrahydro-2H-pyran-4-yl)amino)pyrimidin-4-yl)-1-isopropyl-2-methylquinolin-4(1H)-one

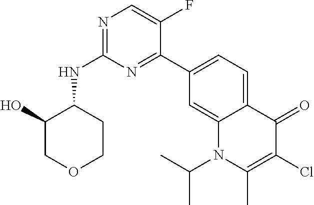 [SMILES: Cc1c(Cl)c(=O)c2ccc(-c3nc(N[C@@H]4CCOC[C@H]4O)ncc3F)cc2n1C(C)C]

The title compound (12 mg, 28%) was prepared in a manner similar to Example 9 step 1 from 7-(5-fluoro-2-(((3S,4R)-3-hydroxytetrahydro-2H-pyran-4-yl)amino)pyrimidin-4-yl)-1-isopropyl-2-methylquinolin-4(1H)-one and N-chlorosuccinimide. ¹H-NMR (300 MHz, DMSO-d6) δ 8.67 (s, 1H), 8.53 (d, J=3.8 Hz, 1H), 8.36 (d, J=8.5 Hz. 1H), 8.03-7.94 (m, 1H), 7.32 (d, J=7.9 Hz, 1H), 5.28-5.12 (m, 1H), 4.96 (d, J=5.2 Hz, 1H), 3.91-3.80 (m, 3H), 3.62-3.48 (m, 1H), 3.39-3.26 (m, 1H), 3.11-2.98 (m, 1H), 2.81 (s, 3H), 2.07-1.97 (m, 1H), 1.78-1.69 (m, 6H), 1.63-1.48 (m, 1H). LC-MS (M+H)⁺=447.2.

### Example 16: 7-(5-fluoro-2-(((3S,4R)-3-hydroxytetrahydro-2H-pyran-4-yl)amino)pyrimidin-4-yl)-1-isopropyl-2,3-dimethylquinolin-4(1H)-one

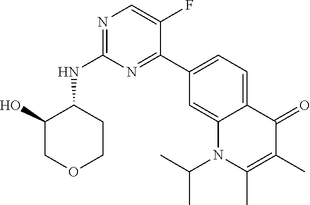 [SMILES: Cc1c(C)n(C(C)C)c2cc(-c3nc(N[C@@H]4CCOC[C@H]4O)ncc3F)ccc2c1=O]

### Step 1: 7-(5-fluoro-2-(((3S,4R)-3-hydroxytetrahydro-2H-pyran-4-yl)aminopyrimidin-4-yl)-3-iodo-1-isopropyl-2-methylquinolin-4(1H)-one

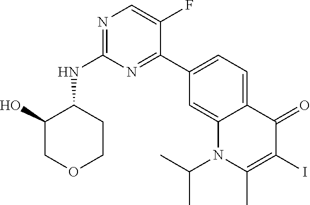 [SMILES: Cc1c(I)c(=O)c2ccc(-c3nc(N[C@@H]4CCOC[C@H]4O)ncc3F)cc2n1C(C)C]

A mixture of 7-(5-fluoro-2-(((3S,4R)-3-hydroxytetrahydro-2H-pyran-4-yl)amino)pyrimidin-4-yl)-1-isopropyl-2-methylquinolin-4(1H)-one (143 mg, 0.345 mmol), iodine (88 mg, 0.345 mmol) and ammonium cerium(IV) nitrate (17 mg, 0.031 mmol) in acetonitrile (4 mL) was stirred for 2 h at 70° C. under nitrogen atmosphere before cooled to room temperature. The solvent was concentrated under reduced pressure. The residue was purified by flash chromatography eluting with methanol in ethyl acetate (0% to 25% gradient) to yield the title compound (105 mg, 56%). LC-MS (M+H)⁺=539.0.

### Step 2: 7-(5-fluoro-2-(((3S,4R)-3-hydroxytetrahydro-2H-pyran-4-yl)amino pyrimidin-4-yl)-1-isopropyl-2,3-dimethylquinolin-4(1H)-one

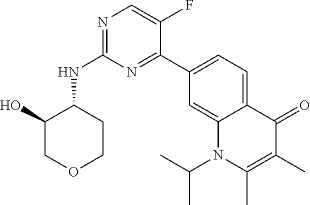 [SMILES: Cc1c(C)n(C(C)C)c2cc(-c3nc(N[C@@H]4CCOC[C@H]4O)ncc3F)ccc2c1=O]

To a mixture of 7-(5-fluoro-2-(((3S,4R)-3-hydroxytetrahydro-2H-pyran-4-yl)amino)pyrimidin-4-yl)-3-iodo-1-isopropyl-2-methylquinolin-4(1H)-one (86 mg, 0.159 mmol) and tetrakis(triphenylphosphine)palladium(0) (18 mg, 0.016 mmol) in dimethylformamide (4 mL) was added 1 M trimethylaluminium solution in tetrahydrofuran (3.18 mL, 3.18 mmol) dropwise at room temperature under nitrogen atmosphere. The resulting mixture was stirred at 80° C. for 3 h under nitrogen atmosphere before cooled to 0° C. and quenched by addition of water (5 mL). The resulting mixture was concentrated under reduced pressure. The residue was purified by flash chromatography eluting with methanol in ethyl acetate (0% to 25% gradient, v/v). The residue was purified by prep-HPLC under the following conditions: column, XBridge Shield RP18 OBD Column, 30×150 mm, 5 um, mobile phase, acetonitrile in water (with 10 mmol/L NH₄HCO₃ and 0.1% NH₃·H₂O), 20% to 50% gradient in 9 min; detector. UV 254 nm. The title compound (33 mg, 48%) obtained. ¹H-NMR (300 MHz, DMSO-d6) δ 8.58 (s, 1H), 8.51-8.45 (m, 1H), 8.34-8.25 (m, 1H), 7.91-7.82 (m, 1H), 7.31-7.22 (m, 1H), 5.15-5.09 (m, 1H), 4.99-4.91 (m, 1H), 3.87-3.81 (m, 3H), 3.55-3.49 (m, 1H), 3.35-3.29 (m, 1H), 3.09-2.96 (m, 1H), 2.49 (s, 3H), 2.08 (s, 3H), 2.02-1.96 (m, 1H), 1.71-1.65 (m, 6H), 1.56-1.46 (m, 1H). LC-MS (M+H)⁺=427.1.

### Example 17: 7-(5-fluoro-2-(((3S,4R)-3-hydroxytetrahydro-2H-pyran-4-yl)amino)pyrimidin-4-yl)-2-(hydroxymethyl)-1-isopropylquinolin-4(1H)-one

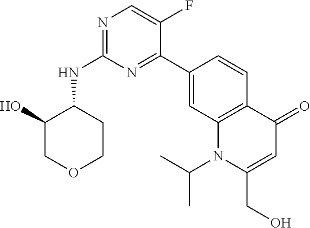 [SMILES: CC(C)n1c(CO)cc(=O)c2ccc(-c3nc(N[C@@H]4CCOC[C@H]4O)ncc3F)cc21]

### Step 1: 7-bromo-1-isopropyl-4-oxo-1,4-dihydroquinoline-2-carbaldehyde

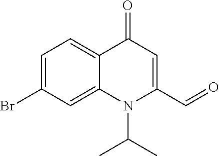 [SMILES: CC(C)n1c(C=O)cc(=O)c2ccc(Br)cc21]

A mixture of 7-bromo-1-isopropyl-2-methylquinolin-4(1H)-one (2.65 g, 9.46 mmol) and selenium dioxide (2.10 g, 18.9 mmol) in 1,4-dioxane (30 mL) was stirred for 16 h at 100° C. under nitrogen atmosphere before cooled to room temperature. The resulting mixture was then quenched by the addition of aqueous saturated sodium bicarbonate solution (30 mL). The organic layer was extracted with ethyl acetate (40 mL×3). The organic phases were combined, washed with brine, dried over sodium sulfate, filtered and concentrated. The residue was purified by flash chromatography eluting with ethyl acetate in petroleum ether (0% to 50% gradient, v/v) to yield the title compound (1.2 g, 43%). LC-MS (M+H+CH4OH)⁺=325.9.

### Step 2: 7-bromo-2-(hydroxymethyl)-1-isopropylquinolin-4(1H)-one

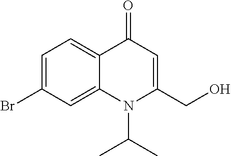 [SMILES: CC(C)n1c(CO)cc(=O)c2ccc(Br)cc21]

To a solution of 7-bromo-1-isopropyl-4-oxo-1,4-dihydroquinoline-2-carbaldehyde (900 mg, 3.06 mmol) in dichloromethane (10 mL) and methanol (20 mL) was added sodium borohydride (174 mg, 4.6 mmol) at 0° C. The mixture was stirred at 25° C. for 0.5 hours before concentration. Saturated ammonium chloride aqueous solution (20 mL) was added. The aqueous layer was extracted with dichloromethane (30 mL×3). The combined organic layers were dried over sodium sulfate, filtered and concentrated in vacuum to give the crude title compound (900 mg) without further purification before next step. LC-MS (M+H)⁺=296.

### Step 3: 2-(hydroxymethyl)-1-isopropyl-7-(4,4,5,5-tetramethyl-1,3,2-dioxaborolan-2-yl)quinolin-4(1H)-one

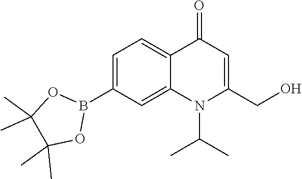 [SMILES: CC(C)n1c(CO)cc(=O)c2ccc(B3OC(C)(C)C(C)(C)O3)cc21]

The title compound (3.5 g, 100%) was prepared in a manner similar to Example 1 step 6 from 7-bromo-2-(hydroxymethyl)-1-isopropylquinolin-4(1H)-one and bis(pinacolato)diboron. LC-MS (M+H)⁺=344.1.

### Step 4: 7-(2-chloro-5-fluoropyrimidin-4-yl)-2-(hydroxymethyl)-1-isopropylquinolin-4(1H)-one

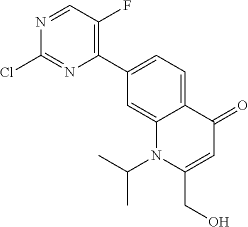 [SMILES: CC(C)n1c(CO)cc(=O)c2ccc(-c3nc(Cl)ncc3F)cc21]

The title compound (3 g, 86%) was prepared in a manner similar to Example 1 step 7 from 2-(hydroxymethyl)-1-isopropyl-7-(4,4,5,5-tetramethyl-1,3,2-dioxaborolan-2-yl)quinolin-4(1H)-one and 2,4-dichloro-5-fluoropyrimidine. LC-MS (M+H)⁺=348.1.

### Step 5: 7-(5-fluoro-2-(((3S,4R)-3-hydroxytetrahydro-2H-pyran-4-yl)amino)pyrimidin-4-yl)-2-(hydroxymethyl)-1-isopropylquinolin-4(1H)-one

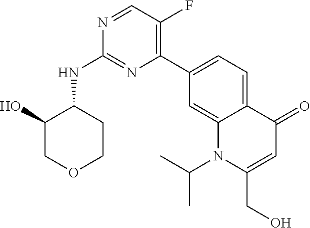 [SMILES: CC(C)n1c(CO)cc(=O)c2ccc(-c3nc(N[C@@H]4CCOC[C@H]4O)ncc3F)cc21]

A mixture of 7-(2-chloro-5-fluoropyrimidin-4-yl)-2-(hydroxymethyl)-1-isopropylquinolin-4(1H)-one (0.5 g, 1.44 mmol), (3S,4R)-4-aminotetrahydro-2H-pyran-3-ol (0.51 g, 4.32 mmol) and N,N-diisopropylethylamine (0.19 g, 1.44 mmol) in dimethylacetamide (15 mL) under nitrogen was stirred at 120° C. for overnight before cooled to room temperature and quenched with water (30 mL). The aqueous layer was extracted with ethyl acetate (50 mL×3). The combined organic phases were dried over sodium sulfate, filtered and concentrated. The residue was purified over silica gel by combi-flash, eluting with methanol in dichloromethane (5% gradient, v/v) to give the title compound (0.6 g, 97%) as off-white solid. ¹H-NMR (400 MHz, DMSO-d6) δ 8.70 (brs, 1H), 8.51 (s, 1H), 8.31 (d, J=8.6 Hz, 1H), 7.93 (d, J=8.8 Hz, 1H), 7.31 (d, J=8.5 Hz, 1H), 6.28 (s, 1H), 5.86 (brs, 1H), 5.15-5.05 (m, 1H), 5.05-4.90 (m, 1H), 4.62 (s, 2H), 3.95-3.80 (m, 3H), 3.54 (s, 1H), 3.45-3.25 (m, 1H), 3.03 (t, J=10.4 Hz, 1H), 2.07-1.97 (m, 1H), 1.76-1.68 (m, 6H), 1.57-1.45 (m, 1H). LC-MS (M+H)⁺=429.2.

### Example 18: 7-(5-fluoro-2-(((3S,4R)-3-hydroxytetrahydro-2H-pyran-4-yl)amino)pyrimidin-4-yl)-1-isopropyl-2-(((R)-2-methylmorpholino)methyl)quinolin-4(1H)-one

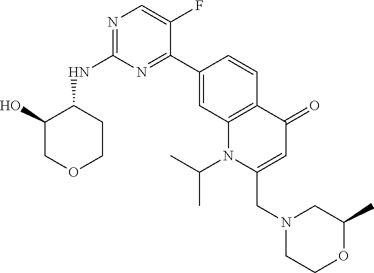 [SMILES: CC(C)n1c(CN2CCO[C@H](C)C2)cc(=O)c2ccc(-c3nc(N[C@@H]4CCOC[C@H]4O)ncc3F)cc21]

### Step 1: 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one

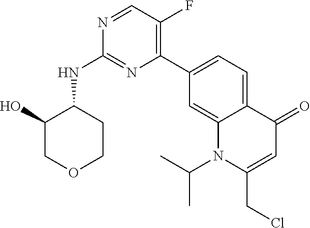 [SMILES: CC(C)n1c(CCl)cc(=O)c2ccc(-c3nc(N[C@@H]4CCOC[C@H]4O)ncc3F)cc21]

7-(5-fluoro-2-(((3S,4R)-3-hydroxytetrahydro-2H-pyran-4-yl)amino)pyrimidin-4-yl)-2-(hydroxymethyl)-1-isopropylquinolin-4(11H)-one (0.6 g, 1.4 mmol) in anhydrous dichloromethane (30 mL) was added thionyl chloride (0.34 g, 2.8 mmol) dropwise at 0° C. Then the resulting solution was stirred at 0° C. for 1 h. The reaction was quenched by addition of saturated sodium bicarbonate aqueous solution (20 mL) and the aqueous phase was extracted with dichloromethane (30 mL×3). The combined organic layers were dried over sodium sulfate, filtered and concentrated. The residue was purified over silica gel by combi-flash, eluting with methanol in dichloromethane (5% gradient, v/v) to give the title compound (0.47 g, 80%). LC-MS (M+H)⁺=447.1

### Step 2: 7-(5-fluoro-2-(((3S,4R)-3-hydroxytetrahydro-2H-pyran-4-yl)amino)pyrimidin-4-yl)-1-isopropyl-2-(((R)-2-methylmorpholino)methyl)quinolin-4(1H)-one

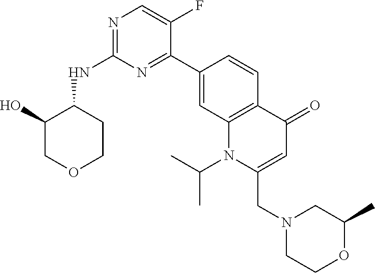 [SMILES: CC(C)n1c(CN2CCO[C@H](C)C2)cc(=O)c2ccc(-c3nc(N[C@@H]4CCOC[C@H]4O)ncc3F)cc21]

A mixture of 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one (25 mg, 0.056 mmol), (R)-2-methylmorpholine (11 mg, 0.112 mmol) and diisopropylethylamine (22 mg, 0.168 mmol) in dimethylformamide (30 mL) under nitrogen was stirred at 50° C. for overnight before cooled to room temperature and addition of water (10 mL). The aqueous layer was extracted with ethyl acetate (50 mL×3). The combined organic layers were dried over sodium sulfate, filtered and concentrated. The residue was purified by Prep-HPLC to give the title compound (14 mg, 49%) as white solid. ¹H-NMR (400 MHz, DMSO-d6) δ 8.75 (s, 1H), 8.52 (s, 1H), 8.31 (d, J=8.5 Hz, 1H), 7.94 (d, J=8.3 Hz, 1H), 7.32 (d, J=8.0 Hz, 1H), 6.18 (s, 11H), 5.45-5.29 (m, 1H), 4.98 (s, 1H), 3.85-3.83 (m, 3H), 3.78-3.76 (m, 1H), 3.69-3.58 (m, 2H), 3.51-3.45 (m, 3H), 3.33-3.28 (m, 1H), 3.04 (t, J=10.3 Hz, 1H), 2.75 (d, J=10.8 Hz, 1H), 2.69 (d, J=11.1 Hz, 1H), 2.16 (t, J=10.7 Hz, 1H), 2.01 (d, J=10.8 Hz, 1H), 1.86 (t, J=10.3 Hz, 1H), 1.74 (s, 6H), 1.53 (d, J=11.4 Hz, 1H), 1.04 (d, J=5.8 Hz, 3H). LC-MS (M+H)⁺=513.1.

### Example 19: 5-fluoro-7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

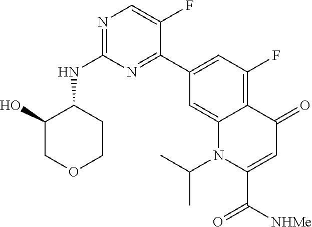 [SMILES: CNC(=O)c1cc(=O)c2c(F)cc(-c3nc(N[C@@H]4CCOC[C@H]4O)ncc3F)cc2n1C(C)C]

### Step 1: 1-(4-bromo-2-fluoro-6-(isopropylamino)phenyl)ethan-1-one

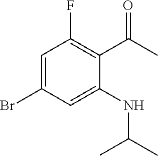

The title compound (700 mg, 100%) was prepared in a manner similar to Example 7 step 1 from 1-(4-bromo-2,6-difluorophenyl)ethan-1-one. LC-MS (M+H)⁺=274.1, 276.1.

### Step 2: ethyl 2-((2-acetyl-5-bromo-3-fluorophenyl)(isopropyl)amino)-2-oxoacetate

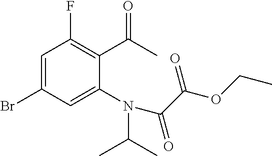 [SMILES: CCOC(=O)C(=O)N(c1cc(Br)cc(F)c1C(C)=O)C(C)C]

The title compound (360 mg, 38%) was prepared in a manner similar to Example 7 step 2 from 1-(4-bromo-2-fluoro-6-(isopropylamino)phenyl)ethan-1-one. LC-MS (M+H)+=374.1, 376.1.

### Step 3: methyl 7-bromo-5-fluoro-1-isopropyl-4-oxo-1,4-dihydroquinoline-2-carboxylate

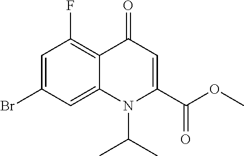 [SMILES: COC(=O)c1cc(=O)c2c(F)cc(Br)cc2n1C(C)C]

The title compound (200 mg, 51%) was prepared in a manner similar to Example 7 step 3 from ethyl 2-((2-acetyl-5-bromo-3-fluorophenyl)(isopropyl)amino)-2-oxoacetate. LC-MS (M+H)⁺=342.1, 344.1.

### Step 4: methyl 5-fluoro-1-isopropyl-4-oxo-7-(4,4,5,5-tetramethyl-1,3,2-dioxaborolan-2-yl)-1,4-dihydroquinoline-2-carboxylate

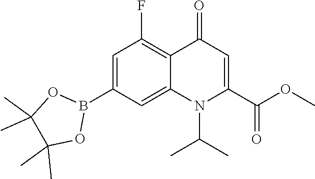

The title compound (226 mg, 100%) was prepared in a manner similar to Example 1 step 6 from methyl 7-bromo-5-fluoro-1-isopropyl-4-oxo-1,4-dihydroquinoline-2-carboxylate and bis(pinacolato)diboron. LC-MS (M+H)⁺=390.1.

### Step 5: methyl 7-(2-chloro-5-fluoropyrimidin-4-yl)-5-fluoro-1-isopropyl-4-oxo-1,4-dihydroquinoline-2-carboxylate

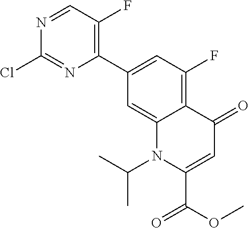 [SMILES: COC(=O)c1cc(=O)c2c(F)cc(-c3nc(Cl)ncc3F)cc2n1C(C)C]

The title compound (160 mg, 73%) was prepared in a manner similar to Example 1 step 7 from methyl 5-fluoro-1-isopropyl-4-oxo-7-(4,4,5,5-tetramethyl-1,3,2-dioxaborolan-2-yl)-1,4-dihydroquinoline-2-carboxylate and 2,4-dichloro-5-fluoropyrimidine. LC-MS (M+H)⁺=394.1.

### Step 6: methyl 5-fluoro-7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carboxylate

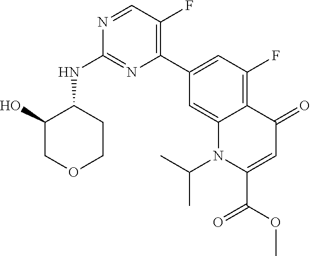 [SMILES: COC(=O)c1cc(=O)c2c(F)cc(-c3nc(N[C@@H]4CCOC[C@H]4O)ncc3F)cc2n1C(C)C]

The title compound (100 mg, 53%) was prepared in a manner similar to Example 2 step 4 from methyl 7-(2-chloro-5-fluoropyrimidin-4-yl)-5-fluoro-1-isopropyl-4-oxo-1,4-dihydroquinoline-2-carboxylate. LC-MS (M+H)⁺=475.1.

### Step 7: 5-fluoro-7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid

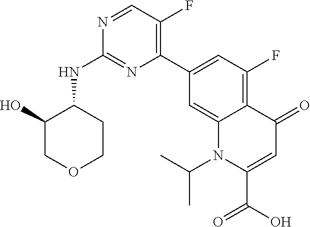 [SMILES: CC(C)n1c(C(=O)O)cc(=O)c2c(F)cc(-c3nc(N[C@@H]4CCOC[C@H]4O)ncc3F)cc21]

The title compound (80 mg, 87%) was prepared in a manner similar to Example 1 step 4 from methyl 5-fluoro-7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carboxylate. LC-MS (M+H)⁺=461.1.

### Step 8: 5-fluoro-7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

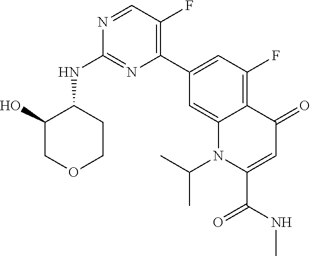 [SMILES: CNC(=O)c1cc(=O)c2c(F)cc(-c3nc(N[C@@H]4CCOC[C@H]4O)ncc3F)cc2n1C(C)C]

The title compound (10 mg, 12%) was prepared in a manner similar to Example 7 step 5 from 5-fluoro-7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid. ¹H-NMR (400 MHz, DMSO-d6) δ 9.02 (d, J=4.6 Hz, 1H), 8.55 (d. J=3.6 Hz, 1H), 8.42 (s, 1H), 7.63 (d, J=12.7 Hz, 1H), 7.38 (d, J=7.6 Hz, 1H), 6.03 (s, 1H), 4.96 (d, J=5.0 Hz, 1H), 4.74-4.62 (m, 1H), 3.96-3.76 (m, 3H), 3.52 (s, 1H), 3.35-3.20 (m, 1H), 3.06-3.01 (m, 1H), 2.80 (d, J=4.5 Hz, 3H), 1.97 (s, 1H), 1.76-1.61 (m, 6H), 1.53-1.50 (m, 1H). LC-MS (M+H)⁺=474.2.

### Example 20: 2-((dimethylamino)methyl)-7-(5-fluoro-2-(((3S,4R)-3-hydroxytetrahydro-2H-pyran-4-yl)amino)pyrimidin-4-yl)-1-isopropylquinolin-4(1H)-one

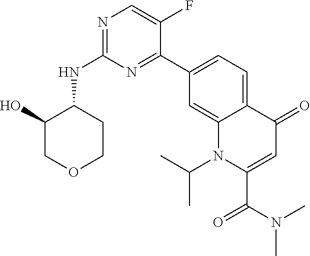 [SMILES: CC(C)n1c(C(=O)N(C)C)cc(=O)c2ccc(-c3nc(N[C@@H]4CCOC[C@H]4O)ncc3F)cc21]

The title compound (12 mg, 19%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and dimethylamine. ¹H-NMR (400 MHz, DMSO-d6) δ 8.72 (s, 1H), 8.51 (d, J=3.8 Hz, 1H), 8.31 (d, J=8.4 Hz, 1H), 7.94 (d, J=8.4 Hz, 1H), 7.31 (d, J=8.0 Hz, 1H), 6.17 (s, 1H), 5.48-5.33 (m, 1H), 4.97 (d, J=5.2 Hz, 1H), 3.90-3.79 (m, 3H), 3.57-3.47 (m, 3H), 3.38-3.28 (m, 1H), 3.10-2.97 (m, 1H), 2.25 (s, 6H), 2.06-1.96 (m, 1H)), 1.76-1.65 (m, 6H), 1.58-1.47 (m, 1H). LC-MS (M+H)⁺=456.2.

### Example 21: 7-(5-chloro-2-(((3S,4R)-3-hydroxytetrahydro-2H-pyran-4-yl)amino)pyrimidin-4-yl)-N-(2-cyanoethyl)-1-isopropyl-4-oxo-1,4-dihydroquinoline-2-carboxamide

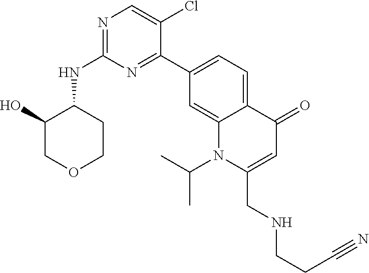 [SMILES: CC(C)n1c(CNCCC#N)cc(=O)c2ccc(-c3nc(N[C@@H]4CCOC[C@H]4O)ncc3Cl)cc21]

The title compound (17 mg, 17%) was prepared in a manner similar to that in Example 7 step 5 from 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid and 3-aminopropanenitrile. ¹H-NMR (400 MHz, DMSO-d6) δ 9.47 (t, J=5.8 Hz, 1H), 8.48 (s, 1H), 8.35-8.21 (m, 2H), 7.88-7.54 (m, 2H), 6.13 (s, 1H), 4.96 (d, J=5.3 Hz, 1H), 4.87-4.72 (m, 1H), 3.88-3.76 (m, 3H), 3.61-3.46 (m, 3H), 3.34-3.19 (m, 1H), 3.01 (s, 1H), 2.84 (t, J=6.3 Hz, 2H), 2.01-1.90 (m, 1H), 1.73-1.64 (m, 6H), 1.57-1.46 (m, 1H). LC-MS (M+H)⁺=511.1.

### Example 22: 7-(5-chloro-2-(((3S,4R)-3-hydroxytetrahydro-2H-pyran-4-yl)amino)pyrimidin-4-yl)-N-(2-hydroxy-2-methylpropyl)-1-isopropyl-4-oxo-1,4-dihydroquinoline-2-carboxamide

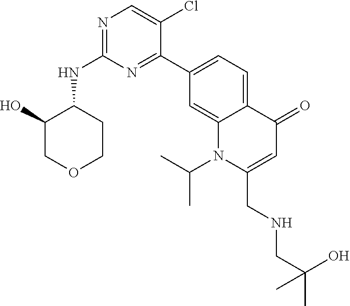 [SMILES: CC(C)n1c(CNCC(C)(C)O)cc(=O)c2ccc(-c3nc(N[C@@H]4CCOC[C@H]4O)ncc3Cl)cc21]

The title compound (7 mg, 7%) was prepared in a manner similar to that in Example 7 step 5 from 7-(5-chloro-2-(((3S,4R)-3-hydroxytetrahydro-2H-pyran-4-yl)amino)pyrimidin-4-yl)-1-isopropyl-4-oxo-1,4-dihydroquinoline-2-carboxylic acid and 1-amino-2-methylpropan-2-ol. ¹H-NMR (400 MHz, DMSO-d6) δ 9.02-8.95 (m, 1H), 8.47 (s, 1H), 8.40-8.13 (m, 2H), 7.93-7.40 (m, 2H), 6.10 (s, 1H), 4.99-4.93 (m, 1H), 4.85-4.73 (m, 1H), 4.60 (s, 1H), 3.86-3.76 (m, 3H), 3.54-3.49 (m, 1H), 3.28-3.23 (m, 3H), 3.04-2.99 (m, 1H), 1.96 (s, 1H), 1.72-1.66 (m, 6H), 1.56-1.48 (m, 1H), 1.16 (s, 6H). LC-MS (M+H)⁺=530.2.

### Example 23: 3-chloro-7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

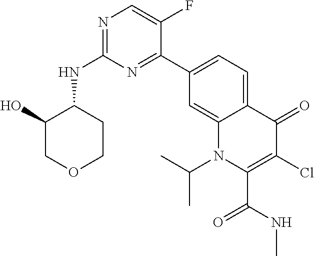 [SMILES: CNC(=O)c1c(Cl)c(=O)c2ccc(-c3nc(N[C@@H]4CCOC[C@H]4O)ncc3F)cc2n1C(C)C]

The title compound (22 mg, 55%) was prepared in a manner similar to that in Example 9 step 1 from 7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide and N-chlorosuccinimide. ¹H-NMR (300 MHz, DMSO-d6) δ 9.22-9.14 (m, 1H), 8.72 (s, 1H), 8.54 (d, J=3.8 Hz, 1H), 8.42 (d, J=8.5 Hz, 1H), 8.05 (d, J=8.5 Hz, 1H), 7.35 (d, J=7.9 Hz, 1H), 4.97 (d, J=5.2 Hz, 1H), 4.81-4.68 (m, 1H), 3.90-3.79 (m, 3H), 3.61-3.45 (m, 1H), 3.38-3.26 (m, 1H), 3.09-2.97 (m, 1H), 2.87 (d, J=4.6 Hz, 3H), 2.05-1.95 (m, 1H), 1.79-1.71 (m, 6H), 1.62-1.44 (m, 1H). LC-MS (M+H)⁺=490.1.

### Example 24: 7-(5-fluoro-2-(((3S,4R)-3-hydroxytetrahydro-2H-pyran-4-yl)amino)pyrimidin-4-yl)-1-isopropyl-N,3-dimethyl-4-oxo-1,4-dihydroquinoline-2-carboxamide

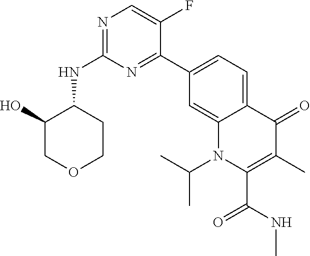 [SMILES: CNC(=O)c1c(C)c(=O)c2ccc(-c3nc(N[C@@H]4CCOC[C@H]4O)ncc3F)cc2n1C(C)C]

### Step 1: 1-(4-bromo-2-(isopropylamino)phenyl)propan-1-one

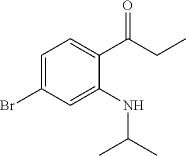

The title compound (934 mg, 79%) was prepared in a manner similar to that in Example 7 step 1 from 1-(4-bromo-2-fluorophenyl)propan-1-one and isopropylamine. LC-MS (M+H)⁺=270.0.

### Step 2: ethyl 2-((5-bromo-2-propionylphenyl)(isopropylamino)-2-oxoacetate

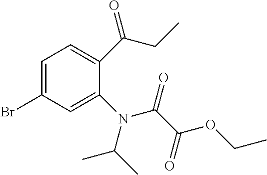 [SMILES: CCOC(=O)C(=O)N(c1cc(Br)ccc1C(=O)CC)C(C)C]

The title compound (976 mg, 76%) was prepared in a manner similar to that in Example 7 step 2 from 1-(4-bromo-2-(isopropylamino)phenyl)propan-1-one and ethyl 2-chloro-2-oxoacetate. LC-MS (M+H)⁺=370.0.

### Step 3: ethyl 7-bromo-1-isoproyl-3-methyl-4-oxo-1,4-dihydroquinolin-2-carboxylate

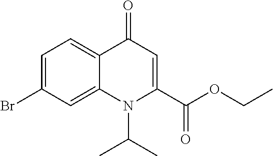 [SMILES: CCOC(=O)c1cc(=O)c2ccc(Br)cc2n1C(C)C]

The title compound (946 mg, 83%) was prepared in a manner similar to that in Example 7 step 3 from ethyl 2-((5-bromo-2-propionylphenyl)(isopropyl)amino)-2-oxoacetate. LC-MS (M+H)⁺=352.0.

### Step 4: ethyl 1-isopropyl-3-methyl-4-oxo-7-(4,4,5,5-tetramethyl-1,3,2-dioxaborolan-2-yl)-1,4-dihydroquinoline-2-carboxylate

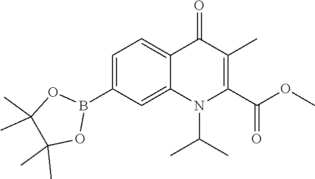

The title compound (646 mg, 63%) was prepared in a manner similar to that in Example 1 step 6 from ethyl 7-bromo-1-isopropyl-3-methyl-4-oxo-1,4-dihydroquinoline-2-carboxylate and bis(pinacolato)diboron. LC-MS (M+H)⁺=400.2.

### Step 5: ethyl 7-(2-chloro-5-fluoropyrimidin-4-yl)-1-isopropyl-3-methyl-4-oxo-1,4-dihydroquinoline-2-carboxylate

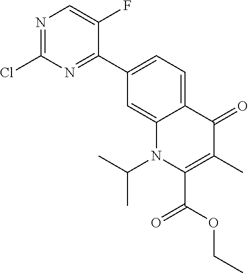 [SMILES: CCOC(=O)c1c(C)c(=O)c2ccc(-c3nc(Cl)ncc3F)cc2n1C(C)C]

The title compound (450 mg, 60%) was prepared in a manner similar to that in Example 1 step 7 from ethyl 1-isopropyl-3-methyl-4-oxo-7-(4,4,5,5-tetramethyl-1,3,2-dioxaborolan-2-yl)-1,4-dihydroquinoline-2-carboxylate and 2,4-dichloro-5-fluoropyrimidine. LC-MS (M+H)⁺=404.2.

### Step 6: ethyl 7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinoline-2-carboxylate

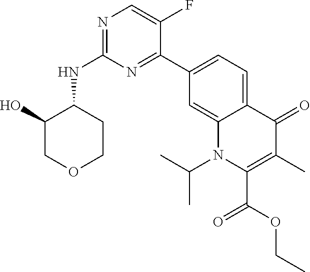 [SMILES: CCOC(=O)c1c(C)c(=O)c2ccc(-c3nc(N[C@@H]4CCOC[C@H]4O)ncc3F)cc2n1C(C)C]

The title compound (190 mg, 71%) was prepared in a manner similar to that in Example 2 step 4 from ethyl 7-(2-chloro-5-fluoropyrimidin-4-yl)-1-isopropyl-3-methyl-4-oxo-1,4-dihydroquinoline-2-carboxylate and (3S,4R)-4-aminooxan-3-ol hydrochloride. LC-MS (M+H)⁺=485.1.

### Step 7: 7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinoline-2-carboxylic acid

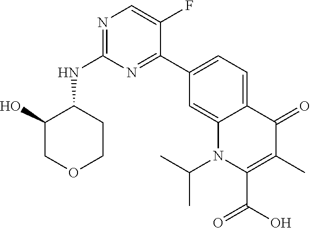 [SMILES: Cc1c(C(=O)O)n(C(C)C)c2cc(-c3nc(N[C@@H]4CCOC[C@H]4O)ncc3F)ccc2c1=O]

The title compound (95 mg, 50%) was prepared in a manner similar to that in Example 1 step 4 from ethyl 7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinoline-2-carboxylate. LC-MS (M+H)⁺=457.2.

### Step 8: 7-(5-fluoro-2-(((3S,4R)-3-hydroxytetrahydro-2H-pyran-4-yl)amino)pyrimidin-4-yl)-1-isopropyl-N,3-dimethyl-4-oxo-1,4-dihydroquinoline-2-carboxamide

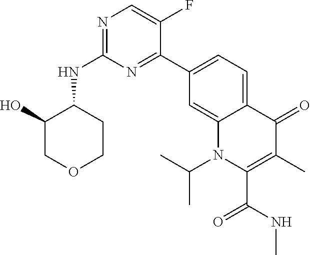 [SMILES: CNC(=O)c1c(C)c(=O)c2ccc(-c3nc(N[C@@H]4CCOC[C@H]4O)ncc3F)cc2n1C(C)C]

The title compound (17 mg, 18%) was prepared in a manner similar to that in Example 7 step 5 from 7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinoline-2-carboxylic acid and methylamine. ¹H-NMR (400 MHz, DMSO-d6) δ 9.06-8.98 (m, 1H), 8.67 (s, 1H), 8.52 (d, J=3.7 Hz, 1H), 8.37 (d, J=8.5 Hz, 1H), 7.96 (d, J=8.4 Hz, 1H), 7.31 (d, J=7.9 Hz, 1H), 5.06-4.96 (m, 1H), 4.74-4.62 (m, 1H), 3.89-3.79 (m, 3H), 3.57-3.50 (m, 1H), 3.35-3.28 (m, 1H), 3.09-2.99 (m, 1H), 2.86 (d, J=4.6 Hz, 3H), 2.05-1.97 (m, 1H), 1.94 (s, 3H), 1.82-1.68 (m, 6H), 1.60-1.45 (m, 1H). LC-MS (M+H)⁺=470.3.

### Example 25: 7-(5-fluoro-2-(((3S,4R)-3-hydroxytetrahydro-2H-pyran-4-yl)amino)pyrimidin-4-yl)-1-isopropyl-2-(morpholinomethyl)quinolin-4(1H)-one

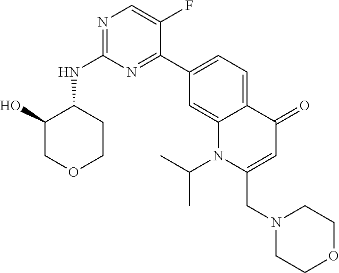 [SMILES: CC(C)n1c(CN2CCOCC2)cc(=O)c2ccc(-c3nc(N[C@@H]4CCOC[C@H]4O)ncc3F)cc21]

The title compound (12 mg, 20%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and morpholine. ¹H-NMR (400 MHz, DMSO-d6) δ: 8.74 (s, 1H), 8.51 (d, J=3.6 Hz, 1H), 8.30 (d. J=8.3 Hz, 1H), 7.94 (d, J=8.5 Hz, 1H), 7.31 (d, J=8.1 Hz, 1H), 6.18 (s, 1H), 5.41-5.34 (m, 1H), 4.97 (d, J=5.0 Hz, 1H), 3.94-3.77 (m, 3H), 3.65 (s, 2H), 3.62-3.46 (m, 5H), 3.36-3.31 (m, 1H), 3.04 (t, J=10.4 Hz, 1H), 2.46 (s, 4H), 2.01 (d, J=12.1 Hz, 1H), 1.74 (t, J=6.5 Hz, 6H), 1.56-1.48 (m, 1H). LC-MS (M+H)⁺=498.

### Example 26: 7-(5-chloro-2-(((3S,4R)-3-hydroxytetrahydro-2H-pyran-4-yl)amino)pyrimidin-4-yl)-1-isopropyl-2-(morpholinomethyl)quinolin-4(1H)-one

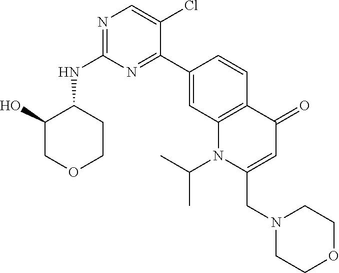 [SMILES: CC(C)n1c(CN2CCOCC2)cc(=O)c2ccc(-c3nc(N[C@@H]4CCOC[C@H]4O)ncc3Cl)cc21]

### Step 1: 7-bromo-2-(hydroxymethyl)-1-isopropylquinolin-4(1H)-one

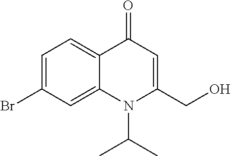 [SMILES: CC(C)n1c(CO)cc(=O)c2ccc(Br)cc21]

To a solution of 7-bromo-1-isopropyl-4-oxo-1,4-dihydroquinoline-2-carbaldehyde (900 mg, 3.06 mmol) in dichloromethane (10 mL) and methanol (20 mL) was added sodium borohydride (174 mg, 4.6 mmol) at 0° C. The mixture was stirred at 25° C. for 0.5 hours before concentration. Saturated ammonium chloride aqueous solution (20 mL) was added. The aqueous layer was extracted with dichloromethane (30 mL×3). The combined organic layers were dried over sodium sulfate, filtered and concentrated in vacuum to give the crude title compound (900 mg) without further purification before next step. LC-MS (M+H)⁺=296.

### Step 2: 7-bromo-2-(chloromethyl)-1-isopropylquinolin-4(1H)-one

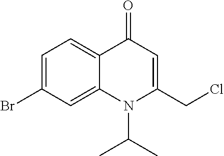 [SMILES: CC(C)n1c(CCl)cc(=O)c2ccc(Br)cc21]

To a solution of 7-bromo-2-(hydroxymethyl)-1-isopropylquinolin-4(1H)-one (900 mg, 3.06 mmol) in dichloromethane (20 mL) was added thionyl chloride (728 mg, 6.12 mmol) at 0° C. The mixture was stirred at 25° C. for 0.5 h under nitrogen atmosphere. The solvent was removed under reduced pressure to give the crude title compound (900 mg). LC-MS (M+H)⁺=314.

### Step 3: 7-bromo-1-isopropyl-2-(morpholinomethyl)quinolin-4(1H)-one

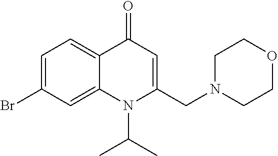 [SMILES: CC(C)n1c(CN2CCOCC2)cc(=O)c2ccc(Br)cc21]

To a mixture of morpholine (83 mg, 0.952 mmol), potassium carbonate (132 mg, 0.952 mmol) and potassium iodide (53 mg, 0.317 mmol) in acetonitrile (3 mL) was added 7-bromo-2-(chloromethyl)-1-isopropylquinolin-4(1H)-one (100 mg, 0.317 mmol). The mixture was stirred at 25° C. for 3 h under nitrogen atmosphere. Water (10 mL) was added and aqueous layer was extracted ethyl acetate (20 mL×3). The combined organic layers were washed with water (10 mL) and brine (10 mL), then dried with over sodium sulfate, filtered and concentrated under reduced pressure. The residue was purified over silica gel by combi-flash, eluting with methanol in dichloromethane (54, v/v) to give the title compound (120 mg, 99%). LC-MS (M+H)⁺=365.

### Step 4: 1-isopropyl-2-(morpholinomethyl)-7-(4,4,5,5-tetramethyl-1,3,2-dioxaborolan-2-yl)quinolin-4(1H)-one

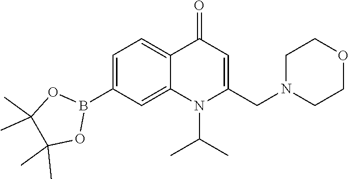 [SMILES: CC(C)n1c(CN2CCOCC2)cc(=O)c2ccc(B3OC(C)(C)C(C)(C)O3)cc21]

The title compound (136 mg, 91%) was prepared in a manner similar to Example 1 step 6 from 7-bromo-1-isopropyl-2-(morpholinomethyl)quinolin-4(1H)-one and bis(pinacolato)diboron. LC-MS (M+H)⁺=413.

### Step 5: 7-(2,5-dichloropyrimidin-4-yl)-1-isopropyl-2-(morpholinomethyl)quinolin-4(1H)-one

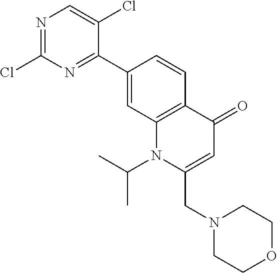 [SMILES: CC(C)n1c(CN2CCOCC2)cc(=O)c2ccc(-c3nc(Cl)ncc3Cl)cc21]

The title compound (122 mg, 85%) was prepared in a manner similar to Example 1 step 7 from 2,4,5-trichloropyrimidine (73 mg, 0.396 mmol, 1.2 eq.) and 1-isopropyl-2-(morpholinomethyl)-7-(4,4,5,5-tetramethyl-1,3,2-dioxaborolan-2-yl)quinolin-4(1H)-one. LC-MS (M+H)⁺=433.

### Step 6: 7-(5-chloro-2-(((3S,4R)-3-hydroxytetrahydro-2H-pyran-4-yl)amino)pyrimidin-4-yl)-1-isopropyl-2-(morpholinomethyl)quinolin-4(1H)-one

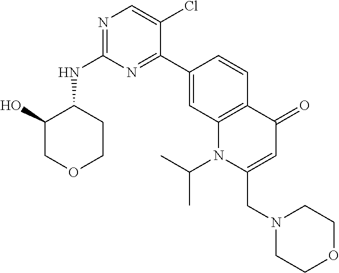 [SMILES: CC(C)n1c(CN2CCOCC2)cc(=O)c2ccc(-c3nc(N[C@@H]4CCOC[C@H]4O)ncc3Cl)cc21]

The title compound (16.2 mg, 11%) was prepared in a manner similar to Example 1 step 8 from 7-(2,5-dichloropyrimidin-4-yl)-1-isopropyl-2-(morpholinomethyl)quinolin-4(1H)-one and (3S,4R)-4-aminotetrahydro-2H-pyran-3-ol hydrochloride. ¹H-NMR (400 MHz, DMSO-d6) δ: 8.47 (s, 1H), 8.38-8.24 (m, 2H), 7.80-7.64 (m, 1H), 7.55 (d, J=8.1 Hz, 1H), 6.18 (s, 1H), 5.39-5.32 (m, 11H), 4.95 (d, J=5.1 Hz, 1H), 3.93-3.75 (m, 3H), 3.65 (s, 2H), 3.58 (s, 4H), 3.51-3.47 (m, 1H), 3.33-3.17 (m, 1H), 3.10-2.87 (m, 1H), 2.46 (s, 4H), 2.07-1.88 (m, 1H), 1.71 (d, J=6.8 Hz, 6H), 1.58-1.40 (m, 1H). LC-MS (M+H)⁺=514.

### Example 27: 7-(5-fluoro-2-(((3S,4R)-3-hydroxytetrahydro-2H-pyran-4-yl)amino)pyrimidin-4-yl)-2-(((S)-3-fluoropyrrolidin-1-yl)methyl)-1-isopropylquinolin-4(1H)-one

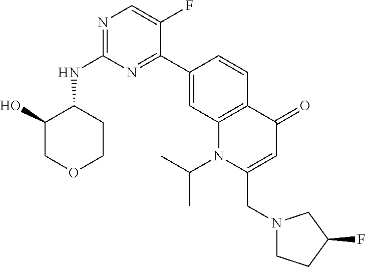 [SMILES: CC(C)n1c(CN2CC[C@H](F)C2)cc(=O)c2ccc(-c3nc(N[C@@H]4CCOC[C@H]4O)ncc3F)cc21]

The title compound (65 mg, 25%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-3-fluoropyrrolidine hydrochloride. ¹H-NMR (400 MHz, DMSO-d6) δ 8.72 (s, 1H). 8.51 (d, J=3.6 Hz, 1H), 8.31 (d, J=8.5 Hz, 11H), 7.94 (d, J=8.4 Hz, 1H). 7.31 (d, J=7.9 Hz, 1H), 6.23 (s, 1H), 5.42-5.33 (m, 1H), 5.30-5.16 (m, 1H), 4.96 (d, J=4.7 Hz, 1H), 3.97-3.82 (m, 3H), 3.80-3.76 (m, 2H), 3.53 (s, 1H), 3.35-3.30 (m, 1H), 3.06-3.01 (m, 1H), 2.96-2.81 (m, 2H), 2.77-2.58 (m, 1H), 2.42-2.38 (m, 1H), 2.30-2.08 (m, 1H), 1.96-1.93 (m, 2H), 1.71 (t, J=6.4 Hz, 6H), 1.63-1.41 (m, 1H). LC-MS (M+H)⁺=500.1.

### Example 28: 2-((8-oxa-3-azabicyclo[3.2.1]octan-3-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

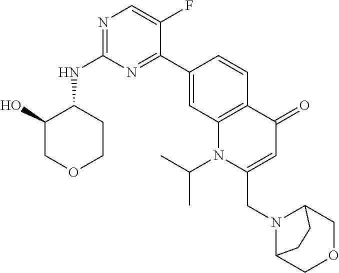 [SMILES: CC(C)n1c(CN2C3CCC2COC3)cc(=O)c2ccc(-c3nc(N[C@@H]4CCOC[C@H]4O)ncc3F)cc21]

The title compound (6 mg, 11%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 8-oxa-3-azabicyclo[3.2.1]octane. ¹H-NMR (400 MHz, DMSO-d6) δ: 8.75 (s, 1H), 8.52 (d, J=3.6 Hz, 1H), 8.30 (d. J=8.4 Hz, 1H), 7.94 (d, J=8.4 Hz, 1H), 7.32 (d, J=7.9 Hz, 1H), 6.14 (s, 1H), 5.52-5.32 (m, 1H), 4.97 (d, J=4.4 Hz, 1H), 4.25 (s, 2H), 4.02-3.78 (m, 3H), 3.53 (s, 3H), 3.36-3.31 (m, 1H), 3.04 (t, J=10.4 Hz, 1H), 2.64 (d, J=10.8 Hz, 2H), 2.33 (d, J=10.6 Hz, 2H), 2.01 (d, J=12.4 Hz, 1H), 1.90-1.65 (m, 10H), 1.62-1.44 (m, 1H). LC-MS (M+H)⁺=524.

### Example 29: 2-((3-oxa-8-azabicyclo[3.2.1]octan-8-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

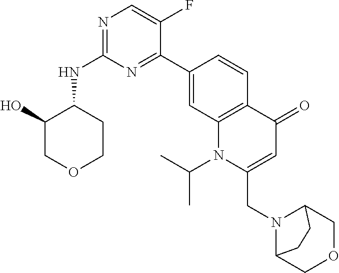 [SMILES: CC(C)n1c(CN2C3CCC2COC3)cc(=O)c2ccc(-c3nc(N[C@@H]4CCOC[C@H]4O)ncc3F)cc21]

The title compound (9.6 mg, 12%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3-oxa-8-azabicyclo[3.2.1]octane. ¹H-NMR (400 MHz, DMSO-d6) δ: 8.76 (s, 1H), 8.52 (d, J=3.6 Hz, 1H), 8.31 (d, J=8.4 Hz, 1H), 7.94 (d, J=8.4 Hz, 1H), 7.31 (d, J=7.9 Hz, 1H), 6.19 (s, 1H), 5.62 (dt, J=14.1, 7.2 Hz, 1H), 4.97 (d, J=4.5 Hz, 1H), 4.04-3.72 (m, 3H), 3.64-3.47 (m, 5H), 3.44 (d, J=10.1 Hz, 2H), 3.36-3.31 (m, 1H), 3.13 (s, 2H), 3.04 (t, J=10.5 Hz, 1H), 1.97 (d, J=23.6 Hz, 3H), 1.78 (t, J=6.1 Hz, 8H), 1.58-1.48 (m, 1H). LC-MS (M+H)⁺=524.

### Example 30: 7-(5-fluoro-2-(((3S,4R)-3-hydroxytetrahydro-2H-pyran-4-yl)amino)pyrimidin-4-yl)-2-((3-hydroxyazetidin-1-yl)methyl)-1-isopropylquinolin-4(1H)-one

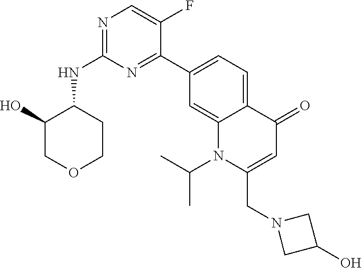 [SMILES: CC(C)n1c(CN2CC(O)C2)cc(=O)c2ccc(-c3nc(N[C@@H]4CCOC[C@H]4O)ncc3F)cc21]

The title compound (20 mg, 62%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and azetidin-3-ol hydrochloride. ¹H-NMR (400 MHz, DMSO-d6) δ 8.64 (s, 1H), 8.50-8.36 (m, 1H), 8.24 (d, J=8.4 Hz, 1H), 7.87 (d, J=8.3 Hz, 1H), 7.26 (d, J=7.9 Hz, 1H), 6.14 (s, 1H), 5.34 (d, J=6.2 Hz, 1H), 5.30-5.13 (m, 1H), 4.92 (d, J=5.1 Hz, 1H), 4.19 (dd, J=12.1, 6.0 Hz, 1H), 3.93-3.74 (m, 3H), 3.68 (s, 2H), 3.53-3.50 (m, 3H), 3.34-3.18 (m, 1H), 2.99 (t, J=10.4 Hz, 1H), 2.87 (t, J=6.4 Hz, 2H), 1.96 (d, J=12.7 Hz, 1H), 1.65 (t, J=6.0 Hz, 6H), 1.47 (dd, J=21.1, 11.3 Hz, 1H). LC-MS (M+H)⁺=484.1.

### Example 31: 7-(5-fluoro-2-(((3S,4R)-3-hydroxytetrahydro-2H-pyran-4-yl)amino)pyrimidin-4-yl)-2-(((3S,4R)-3-fluoro-4-hydroxypyrrolidin-1-yl)methyl)-1-isopropylquinolin-4(1H)-one

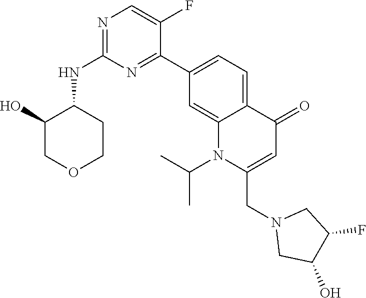 [SMILES: CC(C)n1c(CN2C[C@@H](O)[C@@H](F)C2)cc(=O)c2ccc(-c3nc(N[C@@H]4CCOC[C@H]4O)ncc3F)cc21]

The title compound (22 mg, 32%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R,4S)-4-fluoropyrrolidin-3-ol hydrochloride. ¹H-NMR (400 MHz, DMSO-d6) δ 8.71 (s, 1H), 8.51 (d, J=3.5 Hz, 1H), 8.30 (d, J=8.4 Hz, 1H), 7.93 (d, J=8.3 Hz, 1H), 7.31 (d, J=8.0 Hz, 1H), 6.23 (s, 1H), 5.27 (dt, J=13.9, 6.8 Hz, 1H), 5.14 (s, 1H), 4.96-4.2 (m, 2H), 4.11 (d, J=19.8 Hz, 1H), 3.86-3.77 (m, 5H), 3.53 (s, 1H), 3.35-3.30 (m, 1H), 3.19-2.98 (m, 2H), 2.91 (t, J=7.9 Hz, 1H), 2.78 (dd, J=28.7, 11.7 Hz, 1H), 2.55 (t, J=8.3 Hz, 1H), 2.01 (d, J=12.3 Hz, 1H), 1.70 (t, J=6.3 Hz, 6H), 1.56-1.48 (m, 1H). LC-MS (M+H)⁺=516.3.

### Example 32: 7-(5-fluoro-2-(((3S,4R)-3-hydroxytetrahydro-2H-pyran-4-yl)amino)pyrimidin-4-yl)-2-(((3S,4S)-3-fluoro-4-hydroxypyrrolidin-1-yl)methyl)-1-isopropylquinolin-4(1H)-one

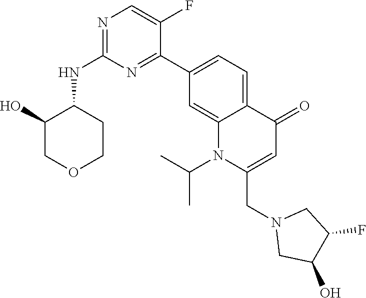 [SMILES: CC(C)n1c(CN2C[C@H](O)[C@@H](F)C2)cc(=O)c2ccc(-c3nc(N[C@@H]4CCOC[C@H]4O)ncc3F)cc21]

The title compound (37 mg, 55%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S,4S)-4-fluoropyrrolidin-3-ol hydrochloride. ¹H-NMR (400 MHz, DMSO-d6) δ 8.72 (s, 1H), 8.51 (d, J=3.3 Hz, 1H), 8.30 (d, J=8.4 Hz, 1H), 7.94 (d, J=8.5 Hz, 1H), 7.31 (d, J=8.0 Hz, 1H), 6.22 (s, 1H), 5.38-5.30 (m, 2H), 4.96 (d, J=4.9 Hz, 1H), 4.84 (d, J=55.7 Hz, 1H), 4.18 (d, J=23.3 Hz, 1H), 3.97-3.78 (m, 4H), 3.72 (d, J=13.6 Hz, 1H), 3.53 (s, 1H), 3.33-3.29 (m, 1H), 3.21-3.10 (m, 1H), 3.03 (t, J=10.4 Hz, 1H), 2.96-2.69 (m, 2H), 2.28 (dd, J=9.0, 5.6 Hz, 1H), 2.01 (d. J=13.9 Hz, 1H), 1.70 (s, 6H), 1.53-1.52 (m, 1H). LC-MS (M+H)⁺=516.3.

### Example 33: 2-(((1R,4R)-2-oxa-5-azabicyclo[2.2.1]heptan-5-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

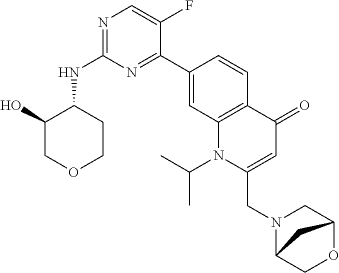 [SMILES: CC(C)n1c(CN2C[C@H]3C[C@@H]2CO3)cc(=O)c2ccc(-c3nc(N[C@@H]4CCOC[C@H]4O)ncc3F)cc21]

The title compound (14 mg, 14%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (1R,4R)-2-oxa-5-azabicyclo[2.2.1]heptane hydrochloride. ¹H-NMR (400 MHz, DMSO-d6) δ: 8.71 (s, 1H), 8.51 (d, J=3.6 Hz, 1H), 8.30 (d, J=8.5 Hz, 1H), 7.93 (d, J 10=8.2 Hz, 1H), 7.31 (d, J=7.8 Hz, 1H), 6.25 (s, 1H), 5.41 (dt, J=14.0, 6.9 Hz, 1H), 4.98 (s, 1H), 4.39 (s, 1H), 3.98-3.76 (m, 6H), 3.68-3.48 (m, 3H), 3.33 (t, J=11.4 Hz, 1H), 3.04 (t, J=10.4 Hz, 1H), 2.81 (d, J=9.7 Hz, 1H), 2.55 (d, J=7.2 Hz, 1H), 2.06-1.96 (m, 1H), 1.83 (d, J=9.5 Hz, 1H), 1.72 (d, J=6.8 Hz, 6H), 1.62 (d, J=9.4 Hz, 1H), 1.58-1.45 (m, 1H). LC-MS (M+H)⁺=510.

### Example 34: 2-(((1S,4S)-2-oxa-5-azabicyclo[2.2.1]heptan-5-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

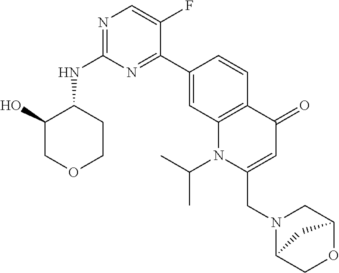 [SMILES: CC(C)n1c(CN2C[C@@H]3C[C@H]2CO3)cc(=O)c2ccc(-c3nc(N[C@@H]4CCOC[C@H]4O)ncc3F)cc21]

The title compound (13 mg, 13%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (1S,4S)-2-oxa-5-azabicyclo[2.2.1]heptane hydrochloride. ¹H-NMR (400 MHz, DMSO-d6) δ: 8.71 (s, 1H), 8.51 (d, J=3.6 Hz, 1H), 8.30 (d, J=8.4 Hz, 1H), 7.93 (d, J=8.3 Hz, 1H), 7.32 (d, J=7.8 Hz, 1H), 6.25 (s, 1H), 5.41 (dt, J=13.8, 6.8 Hz, 1H), 4.98 (s, 1H), 4.39 (s, 1H), 4.02-3.77 (m, 6H), 3.62-3.46 (m, 3H), 3.33 (t, J=11.4 Hz, 1H), 3.04 (t, J=10.4 Hz, 1H), 2.81 (d, J=9.6 Hz, 1H), 2.55 (d, J=9.1 Hz, 1H), 2.01 (d, J=12.8 Hz, 1H), 1.83 (d, J=9.5 Hz, 1H), 1.78-1.65 (m, 6H), 1.62 (d, J=9.4 Hz, 1H), 1.59-1.45 (m, 1H). LC-MS (M+H)⁺=510.

### Example 35: 2-(aminomethyl)-7-(5-fluoro-2-(((3S,4R)-3-hydroxytetrahydro-2H-pyran-4-yl)aminopyrimidin-4-yl)-1-isopropylquinolin-4(1H)-one

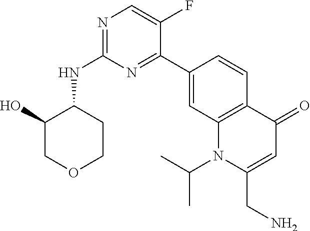 [SMILES: CC(C)n1c(CN)cc(=O)c2ccc(-c3nc(N[C@@H]4CCOC[C@H]4O)ncc3F)cc21]

The title compound (5 mg, 46%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and ammonia. ¹H-NMR (400 MHz, DMSO-d6) δ: 8.69 (s, 1H), 8.51 (d, J=3.6 Hz, 1H), 8.30 (d, J=8.4 Hz, 1H), 7.92 (d, J=8.3 Hz, 1H), 7.31 (d, J=7.8 Hz, 1H), 6.30 (s, 1H), 5.26 (dt, J=14.2, 7.0 Hz, 1H), 4.95 (s, 1H), 3.97-3.77 (m, 5H), 3.58-3.48 (m, 1H). 3.32 (t, J=11.5 Hz, 1H), 3.03 (t, J=10.4 Hz, 1H), 2.01 (d, J=13.9 Hz, 1H), 1.72 (t, J=5.8 Hz, 6H), 1.59-1.44 (m, 1H). LC-MS (M+H)⁺=428.

### Example 36: 5-chloro-7-(5-fluoro-2-(((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

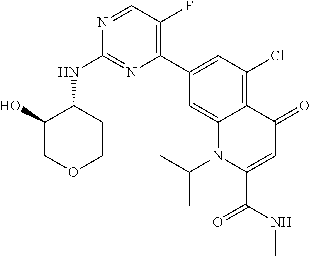 [SMILES: CNC(=O)c1cc(=O)c2c(Cl)cc(-c3nc(N[C@@H]4CCOC[C@H]4O)ncc3F)cc2n1C(C)C]

### Step 1: 1-(4-bromo-2-chloro-6-(isopropylamino)phenyl)ethan-1-one

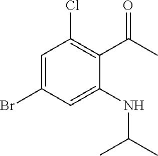

The title compound (200 mg, 85%) was prepared in a manner similar to Example 7 step 1 from 1-(4-bromo-2-chloro-6-fluorophenyl)ethan-1-one. LC-MS (M+H)⁺=290.0, 292.0.

### Step 2: ethyl 2-((2-acetyl-5-bromo-3-chlorophenyl)(isopropyl)amino)-2-oxoacetate

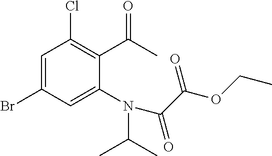 [SMILES: CCOC(=O)C(=O)N(c1cc(Br)cc(Cl)c1C(C)=O)C(C)C]

The title compound (200 mg, 88%) was prepared in a manner similar to Example 7 step 2 from 1-(4-bromo-2-chloro-6-(isopropylamino)phenyl)ethan-1-one. LC-MS (M+H)⁺=390.0, 392.0.

### Step 3: methyl 7-bromo-5-chloro-1-isopropyl-4-oxo-1,4-dihydroquinoline-2-carboxylate

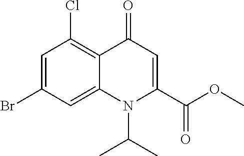 [SMILES: COC(=O)c1cc(=O)c2c(Cl)cc(Br)cc2n1C(C)C]

The title compound (150 mg, 92%) was prepared in a manner similar to Example 7 step 3 from ethyl 2-((2-acetyl-5-bromo-3-chlorophenyl)(isopopyl)amino)-2-oxoacetate. LC-MS (M+H)⁺=358.0, 360.0.

### Step 4: 7-bromo-5-chloro-1-isopropyl-4-oxo-1,4-dihydroquinoline-2-carboxylic acid

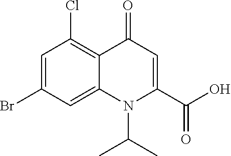 [SMILES: CC(C)n1c(C(=O)O)cc(=O)c2c(Cl)cc(Br)cc21]

The title compound (120 mg, 78%) was prepared in a manner similar to Example 1 step 4 from methyl 7-bromo-5-chloro-1-isopropyl-4-oxo-1,4-dihydroquinoline-2-carboxylate. LC-MS (M+H)⁺=344.0, 346.0.

### Step 5: 7-bromo-5-chloro-1-isopropyl-N-methyl-4-oxo-1,4-dihydroquinoline-2-carboxamide

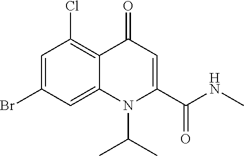 [SMILES: CNC(=O)c1cc(=O)c2c(Cl)cc(Br)cc2n1C(C)C]

The title compound (100 mg, 81%) was prepared in a manner similar to Example 7 step 5 from 7-bromo-5-chloro-1-isopropyl-4-oxo-1,4-dihydroquinoline-2-carboxylic acid and methylamine. LC-MS (M+H)⁺=357.0, 359.0.

### Step 6: 5-chloro-1-isopropyl-N-methyl-4-oxo-7-(4,4,5,5-tetramethyl-1,3,2-dioxaborolan-2-yl)-1,4-dihydroquinoline-2-carboxamide

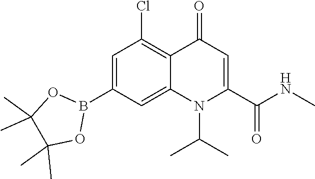

The title compound (100 mg, 88%) was prepared in a manner similar to Example 1 step 6 from 7-bromo-5-chloro-1-isopropyl-N-methyl-4-oxo-1,4-dihydroquinoline-2-carboxamide and bis(pinacolato)diboron. LC-MS (M+H)⁺=405.1.

### Step 7: 5-chloro-7-(2-chloro-5-fluoropyrimidin-4-yl)-1-isopropyl-N-methyl-4-oxo-1,4-dihydroquinoline-2-carboxamide

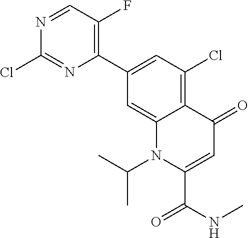 [SMILES: CNC(=O)c1cc(=O)c2c(Cl)cc(-c3nc(Cl)ncc3F)cc2n1C(C)C]

The title compound (60 mg, 59%) was prepared in a manner similar to Example 1 step 7 from 5-chloro-1-isopropyl-N-methyl-4-oxo-7-(4,4,5,5-tetramethyl-1,3,2-dioxaborolan-2-yl)-1,4-dihydroquinoline-2-carboxamide and 2,4-dichloro-5-fluoropyrimidine. LC-MS (M+H)⁺=409.1.

### Step 8: 5-chloro-7-(5-fluoro-2-((3S,4R)-3-hydroxytetrahydro-2H-pyran-4-yl)amino)pyrimidin-4-yl)-1-isopropyl-N-methyl-4-oxo-1,4-dihydroquinoline-2-carboxamide

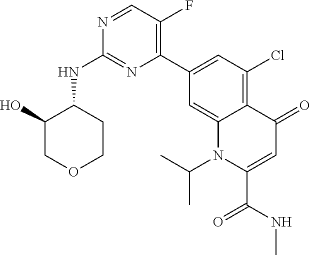 [SMILES: CNC(=O)c1cc(=O)c2c(Cl)cc(-c3nc(N[C@@H]4CCOC[C@H]4O)ncc3F)cc2n1C(C)C]

The title compound (5 mg, 8%) was prepared in a manner similar to Example 2 step 4 from 5-chloro-7-(2-chloro-5-fluoropyrimidin-4-yl)-1-isopropyl-N-methyl-4-oxo-1,4-dihydroquinoline-2-carboxamide and (3S,4R)-4-aminotetrahydro-2H-pyran-3-ol hydrochloride. ¹H-NMR (400 MHz, DMSO-d6) δ 9.02 (d, J=4.6 Hz, 1H), 8.55 (d, J=3.2 Hz, 2H), 7.92 (s, 1H), 7.40 (d, J=8.1 Hz, 1H), 6.09 (s, 1H), 4.96 (d, J=4.7 Hz, 1H), 4.75-4.57 (m, 1H), 3.95-3.77 (m, 3H), 3.52 (s, 1H), 3.31-3.26 (m, 1H), 3.03 (t, J=10.6 Hz, 1H), 2.80 (d, J=4.4 Hz, 3H), 1.97 (s, 1H), 1.68 (d, J=3.6 Hz, 6H), 1.54-1.51 (m, 1H). LC-MS (M+H)⁺=490.1.

### Example 37: 1-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)piperidine-4-carbonitrile

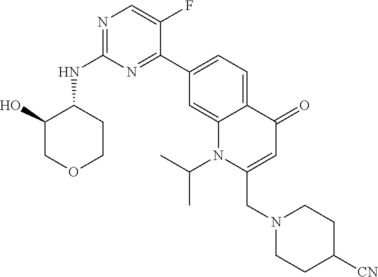 [SMILES: CC(C)n1c(CN2CCC(C#N)CC2)cc(=O)c2ccc(-c3nc(N[C@@H]4CCOC[C@H]4O)ncc3F)cc21]

### Step 1: 7-bromo-2-(1,3-dioxolan-2-yl)-1-isopropylquinolin-4(1H)-one

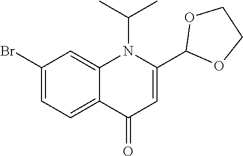 [SMILES: CC(C)n1c(C2OCCO2)cc(=O)c2ccc(Br)cc21]

A mixture of 7-bromo-1-isopropyl-4-oxoquinoline-2-carbaldehyde (1.0 g, 3.40 mmol) and ethylene glycol (740 mg, 11.900 mmol) in toluene (10 mL) was stirred for 12 h at 135° C. under nitrogen atmosphere before cooled to room temperature and quenched by addition of water (20 mL). The aqueous solution was extracted with ethyl acetate (20 mL×3). The combined organic phases were washed with brine, dried over sodium sulfate and filtered. The solvent was concentrated under reduced pressure to yield the title compound (550 mg, 47%). LC-MS (M+H)⁺=337.9.

### Step 2: 2-(1,3-dioxolan-2-yl)-1-isopropyl-7-(4,4,5,5-tetramethyl-1,3,2-dioxaborolan-2-yl)quinolin-4(1H)-one

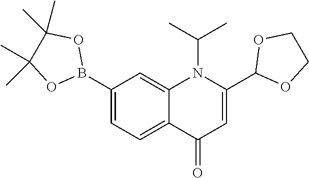 [SMILES: CC(C)n1c(C2OCCO2)cc(=O)c2ccc(B3OC(C)(C)C(C)(C)O3)cc21]

The title compound (380 mg, 60%) was prepared in a manner similar to that in Example 1 step 6 from 7-bromo-2-(1,3-dioxolan-2-yl)-1-isopropylquinolin-4(1H)-one and bis(pinacolato)diboron. LC-MS (M+H)⁺=386.1.

### Step 3: 7-(2-chloro-5-fluoropyrimidin-4-yl)-2-(1,3-dioxolan-2-yl)-1-isopropylquinolin-4(1H)-one

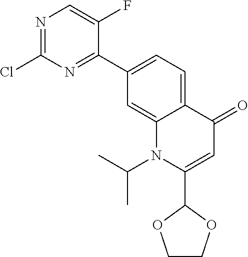 [SMILES: CC(C)n1c(C2OCCO2)cc(=O)c2ccc(-c3nc(Cl)ncc3F)cc21]

The title compound (210 mg, 54%) was prepared in a manner similar to that in Example 1 step 7 from 2-(1,3-dioxolan-2-yl)-1-isopropyl-7-(4,4,5,5-tetramethyl-1,3,2-dioxaborolan-2-yl)quinolin-4(1H)-one and 2,4-dichloro-5-fluoropyrimidine. LC-MS (M+H)⁺=390.0.

### Step 4: 2-(1,3-dioxolan-2-yl)-7-(5-fluoro-2-(((3S,4R)-3-hydroxytetrahydro-2H-pyran-4-yl)amino)pyrimidin-4-yl)-1-isopropylquinolin-4(1H)-one

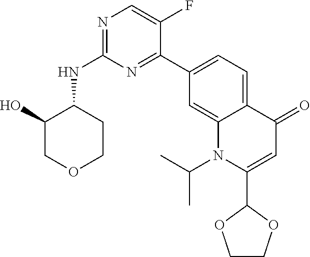 [SMILES: CC(C)n1c(C2OCCO2)cc(=O)c2ccc(-c3nc(N[C@@H]4CCOC[C@H]4O)ncc3F)cc21]

The title compound (120 mg, 47%) was prepared in a manner similar to that in Example 2 step 4 from 7-(2-chloro-5-fluoropyrimidin-4-yl)-2-(1,3-dioxolan-2-yl)-1-isopropylquinolin-4(1H)-one and (3S,4R)-4-aminooxan-3-ol hydrochloride. LC-MS (M+H)⁺=471.1.

### Step 5: 7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carbaldehyde

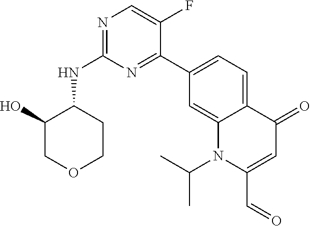 [SMILES: CC(C)n1c(C=O)cc(=O)c2ccc(-c3nc(N[C@@H]4CCOC[C@H]4O)ncc3F)cc21]

A mixture of 7-(5-fluoro-2-(((3S,4R)-3-hydroxytetrahydro-2H-pyran-4-yl)amino)pyrimidin-4-yl)-1-isopropyl-4-oxo-1,4-dihydroquinoline-2-carbaldehyde (120 mg, 0.270 mmol) and iron(III) p-toluenesulfonate (2.90 g, 5.100 mmol) in water (5 mL) was stirred for 16 h at 100° C. under nitrogen atmosphere before cooled to room temperature and quenched by addition of water (10 mL). The resulting aqueous layer was extracted with ethyl acetate (20 mL×3). The organic phases were combined, washed with brine, dried over sodium sulfate, filtered and concentrated. The residue was purified by flash chromatography eluting with methanol in dichloromethane (0% to 15% gradient, v/v) to yield the title compound (46 mg, 42%). LC-MS (M+H+CH4OH)⁺=459.1.

### Step 6: 1-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)piperidine-4-carbonitrile

To a mixture of 7-(5-fluoro-2-{[(3S,4R)-3-hydroxyoxan-4-yl]amino}pyrimidin-4-yl)-1-isopropyl-4-oxoquinoline-2-carbaldehyde (15 mg, 0.036 mmol) in dichloromethane (2 mL) was added piperidine-4-carbonitrile (14 mg, 0.129 mmol) dropwise at room temperature under nitrogen atmosphere. The resulting mixture was stirred for 2 h at room temperature under nitrogen atmosphere. To the above mixture was added sodium triacetoxyborohydride (11 mg, 0.054 mmol) in portions at room temperature. The resulting mixture was stirred for additional 16 h at room temperature before concentrated under reduced pressure. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₃·H₂O), 22% to 50% gradient in 8 min; detector, UV 254 nm. The title compound (12 mg, 19%) was obtained. ¹H-NMR (300 MHz, DMSO-d6) δ 8.75 (brs, 1H), 8.52 (d, J=3.8 Hz, 1H), 8.31 (d, J=8.4 Hz, 1H), 7.98-7.91 (m, 1H), 7.33 (d, J=8.0 Hz, 1H), 6.19 (s, 1H), 5.37-5.26 (m, 1H), 4.99 (d, J=5.2 Hz, 1H), 3.93-3.80 (m, 3H), 3.65 (s, 2H), 3.60-3.49 (m, 1H), 3.39-3.28 (m, 1H), 3.10-3.00 (m, 1H), 2.94-2.89 (m, 1H), 2.66-2.61 (m, 2H), 2.41-2.36 (m, 2H), 2.06-1.98 (m, 1H), 1.90-1.84 (m, 2H), 1.77-1.66 (m, 8H), 1.60-1.46 (m, 1H). LC-MS (M+H)⁺=521.3.

### Example 38: 7-(5-fluoro-2-(((3S,4R)-3-hydroxytetrahydro-2H-pyran-4-yl)amino)pyrimidin-4-yl)-2-((4-hydroxy-4-methylpiperidin-1-yl)methyl)-1-isopropylquinolin-4(1H)-one

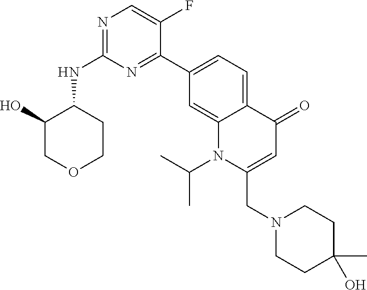 [SMILES: CC(C)n1c(CN2CCC(C)(O)CC2)cc(=O)c2ccc(-c3nc(N[C@@H]4CCOC[C@H]4O)ncc3F)cc21]

The title compound (7 mg, 19%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4-methylpiperidin-4-ol. ¹H-NMR (400 MHz, DMSO-d6) δ 8.74 (brs, 1H), 8.52 (d, J=3.8 Hz, 1H), 8.31 (d, J=8.4 Hz, 1H), 7.97-7.91 (m, 1H), 7.33 (d, J=7.9 Hz, 1H), 6.17 (s, 1H), 5.44-5.32 (m, 1H), 4.98 (d, J=5.3 Hz, 1H), 4.19 (s, 1H), 3.90-3.81 (m, 3H), 3.62 (s, 2H), 3.60-3.48 (m, 1H), 3.39-3.28 (m, 1H), 3.09-2.99 (m, 1H), 2.48-2.44 (m, 4H), 2.06-1.98 (m, 1H), 1.77-1.68 (m, 6H), 1.57-1.40 (m, 5H), 1.11 (s, 3H). LC-MS (M+H)⁺=526.3.

### Example 39: 7-(5-fluoro-2-(((3S,4R)-3-hydroxytetrahydro-2H-pyran-4-yl)amino)pyrimidin-4-yl)-2-((4-hydroxypiperidin-1-yl)methyl)-1-isopropylquinolin-4(1H)-one

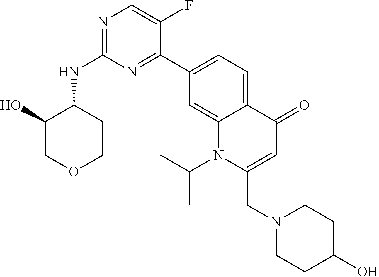 [SMILES: CC(C)n1c(CN2CCC(O)CC2)cc(=O)c2ccc(-c3nc(N[C@@H]4CCOC[C@H]4O)ncc3F)cc21]

The title compound (15 mg, 45%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and piperidin-4-ol. ¹H-NMR (300 MHz, DMSO-d6) δ 8.71 (s, 1H), 8.50 (d, J=3.8 Hz, 1H), 8.29 (d. J=8.4 Hz, 1H), 7.97-7.88 (m, 1H), 7.29 (d, J=7.9 Hz, 1H), 6.14 (s, 1H), 5.42-5.27 (m, 1H), 4.95 (d, J=5.2 Hz, 1H), 4.58 (d, J=4.1 Hz, 1H), 3.90-3.78 (m, 3H), 3.64-3.45 (m, 4H), 3.39-3.25 (m, 1H), 3.09-2.96 (m, 1H), 2.72 (d, J=10.9 Hz, 2H), 2.20-2.08 (m, 2H), 2.06-1.95 (m, 1H), 1.72 (s, 8H), 1.57-1.33 (m, 3H). LC-MS (M+H)⁺=512.3.

### Example 40: 3-cyclopropyl-7-(5-fluoro-2-(((3S,4R)-3-hydroxytetrahydro-2H-pyran-4-yl)amino)pyrimidin-4-yl)-1-isopropyl-2-methylquinolin-4(1H)-one

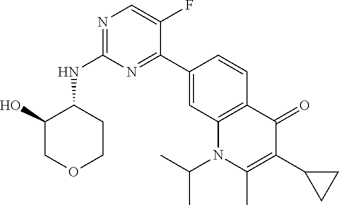 [SMILES: Cc1c(C2CC2)c(=O)c2ccc(-c3nc(N[C@@H]4CCOC[C@H]4O)ncc3F)cc2n1C(C)C]

### Step 1: 3-bromo-7-(5-fluoro-2-(((3S,4R)-3-hydroxytetrahydro-2H-pyran-4-yl)amino)pyrimidin-4-yl)-1-isopropyl-2-methylquinolin-4(1H)-one

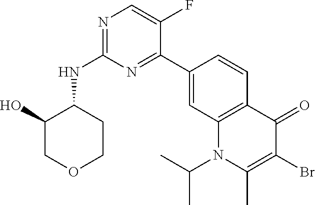 [SMILES: Cc1c(Br)c(=O)c2ccc(-c3nc(N[C@@H]4CCOC[C@H]4O)ncc3F)cc2n1C(C)C]

The title compound (1.2 g, 84%) was prepared in a manner similar to that in Example 9 step 1 from 7-(5-fluoro-2-(((3S,4R)-3-hydroxytetrahydro-2H-pyran-4-yl)amino)pyrimidin-4-yl)-1-isopropyl-2-methylquinolin-4(1H)-one (1.2 g, 2.9 mmol). LC-MS (M+H)⁺=491.1, 493.1.

### Step 2: 3-cyclopropyl-7-(5-fluoro-2-(((3S,4R)-3-hydroxytetrahydro-2H-pyran-4-yl)amino)pyrimidin-4-yl)-1-isopopyl-2-methylquinolin-4(1H)-one

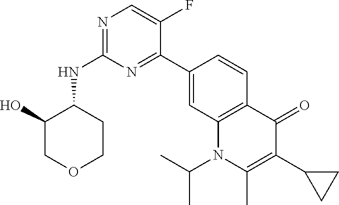 [SMILES: Cc1c(C2CC2)c(=O)c2ccc(-c3nc(N[C@@H]4CCOC[C@H]4O)ncc3F)cc2n1C(C)C]

The title compound (20 mg, 46%) was prepared in a manner similar to that in Example 1 step 7 from 3-bromo-7-(5-fluoro-2-(((3S,4R)-3-hydroxytetrahydro-2H-pyran-4-yl)amino)pyrimidin-4-yl)-1-isopropyl-2-methylquinolin-4(1H)-one and cyclopropylboronic acid. ¹H-NMR (400 MHz, DMSO-d6) δ 8.59 (brs, 1H), 8.50 (d, J=3.6 Hz, 1H), 8.27 (d, J=8.4 Hz, 1H), 7.87 (d, J=8.3 Hz, 1H), 7.29 (d, J=7.9 Hz, 1H), 5.18-5.08 (m, 1H), 4.97 (d, J=4.6 Hz, 1H), 3.94-3.78 (m, 3H), 3.58-3.48 (m, 1H), 3.37-3.27 (m, 1H), 3.03 (t. J=10.5 Hz, 1H), 2.74 (s, 3H), 2.06-1.96 (m, 1H), 1.73-1.65 (m, 6H), 1.57-1.44 (m, 2H), 0.96-0.89 (m, 2H), 0.55-0.48 (m, 2H). LC-MS (M+H)⁺=453.2.

### Example 41: 7-(5-fluoro-2-(((3S,4R)-3-hydroxytetrahydro-2H-pyran-4-yl)amino)pyrimidin-4-yl)-1-isopropyl-2-(((3aR,6aS)-tetrahydro-1H-furo[3,4-c]pyrrol-5(3H)-yl)methyl)quinolin-4(1H)-one

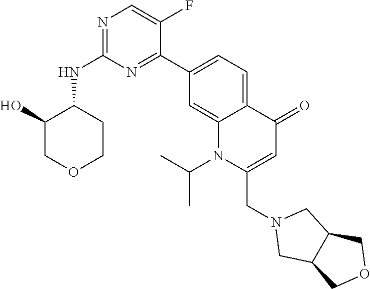 [SMILES: CC(C)n1c(CN2C[C@H]3COC[C@H]3C2)cc(=O)c2ccc(-c3nc(N[C@@H]4CCOC[C@H]4O)ncc3F)cc21]

The title compound (12 mg, 41%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3aR,6aS)-hexahydro-1H-furo[3,4-c]pyrrole hydrochloride. ¹H-NMR (400 MHz, DMSO-d6) δ 8.74 (s, 1H), 8.52 (s, 1H), 8.30 (d, J=8.2 Hz, 1H), 7.94 (d, J=8.2 Hz, 1H), 7.32 (d, J=7.9 Hz, 1H), 6.19 (s, 1H), 5.62-5.30 (m, 1H), 4.97 (d, J=3.9 Hz, 1H), 4.00-3.77 (m, 5H), 3.69 (s, 2H), 3.53 (s, 1H), 3.33 (s, 5H), 3.04 (t, J=10.5 Hz, 1H), 2.73 (s, 2H), 2.56 (d, J=9.0 Hz, 2H), 2.01 (d, J=11.5 Hz, 1H), 1.72 (t. J=6.4 Hz, 6H), 1.52 (d, J=12.1 Hz, 1H). LC-MS (M+H)⁺=524.0.

### Example 42: 2-((3,3-dimethylpyrrolidin-1-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

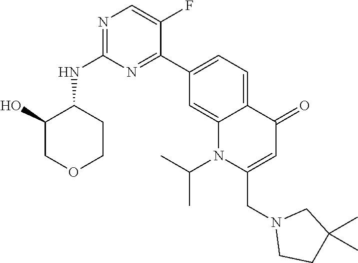 [SMILES: CC(C)n1c(CN2CCC(C)(C)C2)cc(=O)c2ccc(-c3nc(N[C@@H]4CCOC[C@H]4O)ncc3F)cc21]

The title compound (9 mg, 32%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3,3-dimethylpyrrolidine. ¹H-NMR (400 MHz, DMSO-d6) δ 8.73 (s, 1H), 8.52 (s, 1H), 8.30 (d, J=8.3 Hz, 11H), 7.94 (d, J=8.4 Hz, 1H), 7.31 (d, J=7.8 Hz, 1H), 6.18 (s, 1H), 5.62-5.35 (m, 1H), 4.97 (s, 1H), 3.84 (d, J=10.8 Hz, 3H), 3.70 (s, 2H), 3.53 (s, 1H), 3.43-3.30 (m, 1H), 3.04 (t, J=10.4 Hz, 1H), 2.64 (s, 2H), 2.34 (s, 2H), 2.01 (d, J=11.1 Hz, 1H), 1.73 (t, J=6.1 Hz, 6H), 1.65-1.45 (m, 3H), 1.06 (s, 6H). LC-MS (M+H)⁺=510.0.

### Example 43: 2-((2-azabicyclo[2.1.1]hexan-2-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

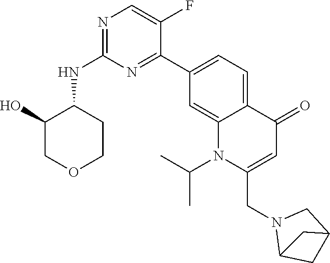 [SMILES: CC(C)n1c(CN2CC3CC2C3)cc(=O)c2ccc(-c3nc(N[C@@H]4CCOC[C@H]4O)ncc3F)cc21]

The title compound (6 mg, 22%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 2-azabicyclo[2.1.1]hexane. ¹H-NMR (400 MHz, DMSO-d6) δ 8.73 (s, 1H), 8.52 (s, 1H), 8.31 (d, J=8.3 Hz, 1H), 7.93 (d, J=8.5 Hz, 1H), 7.31 (d, J=7.8 Hz, 1H), 6.26 (s, 1H), 5.57 (s, 1H), 4.97 (s, 1H), 3.85 (s, 5H), 3.53 (s, 1H), 3.43 (d, J=5.3 Hz, 1H), 3.34 (s, 1H), 3.04 (s, 1H), 2.74 (s, 1H), 2.66 (s, 2H), 2.00 (s, 1H), 1.71 (d, J=14.9 Hz, 8H), 1.52 (d, J=11.7 Hz, 1H), 1.45 (s, 2H). LC-MS (M+H)⁺=494.0.

### Example 44: 2-((1-oxa-8-azaspiro[4.5]decan-8-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

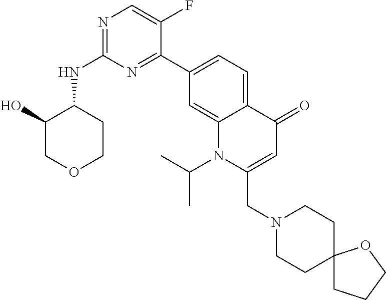 [SMILES: CC(C)n1c(CN2CCC3(CCCO3)CC2)cc(=O)c2ccc(-c3nc(N[C@@H]4CCOC[C@H]4O)ncc3F)cc21]

The title compound (7 mg, 22%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 1-oxa-8-azaspiro[4.5]decane. ¹H-NMR (400 MHz, DMSO-d6) δ 8.80 (s, 1H), 8.58 (s, 1H), 8.37 (d, J=7.9 Hz, 1H), 8.00 (d, J=8.2 Hz, 1H), 7.38 (d, J=7.7 Hz, 1H), 6.23 (s, 1H), 5.44 (s, 1H), 5.04 (s, 1H), 3.92 (s, 3H), 3.76 (s, 2H), 3.68 (s, 2H), 3.65-3.45 (m, 3H), 3.10 (t, J=10.1 Hz, 11H), 2.50 (s, 2H), 2.06 (s, 1H), 1.90 (s, 3H), 1.79 (s, 6H), 1.71 (s, 2H), 1.62 (s, 5H). LC-MS (M+H)⁺=552.0.

### Example 45: 7-(5-fluoro-2-(((3S,4R)-3-hydroxytetrahydro-2H-pyran-4-yl)amino)pyrimidin-4-yl)-2-(((3R,4S)-3-fluoro-4-hydroxypyrrolidin-1-yl)methyl)-1-isopropylquinolin-4(1H)-one

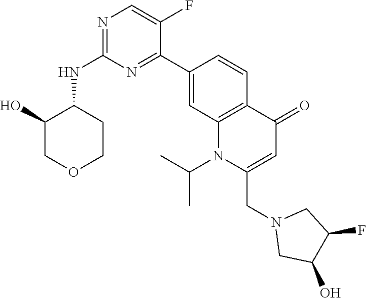 [SMILES: CC(C)n1c(CN2C[C@@H](F)[C@@H](O)C2)cc(=O)c2ccc(-c3nc(N[C@@H]4CCOC[C@H]4O)ncc3F)cc21]

The title compound (12 mg, 41%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S,4R)-4-fluoropyrrolidin-3-ol hydrochloride. ¹H-NMR (400 MHz, DMSO-d6) δ 8.71 (s, 1H), 8.52 (s, 1H), 8.30 (d, J=8.1 Hz, 1H), 7.93 (d, J=8.1 Hz, 1H), 7.31 (d.J=7.3 Hz, 1H), 6.23 (s, 1H), 5.38-5.20 (m, 1H), 5.14 (d, J=5.8 Hz, 1H), 4.96 (s, 1H), 4.89 (d, J=57.1 Hz, 1H), 4.13-4.09 (m, 1H), 3.85-3.77 (m, 5H), 3.53 (s, 1H), 3.30 (s, 1H), 3.16-3.01 (m, 2H), 2.91 (t, J=8.3 Hz, 1H), 2.78 (dd, J=28.5, 11.3 Hz, 1H), 2.57-2.55 (m, 1H), 1.99 (s, 1H), 1.70 (s, 6H), 1.54-1.51 (m, 1H). LC-MS (M+H)⁺=516.1.

### Example 46: 7-(5-fluoro-2-(((3S,4R)-3-hydroxytetrahydro-2H-pyran-4-yl)amino)pyrimidin-4-yl)-2-(((3R,4R)-3-fluoro-4-hydroxypyrrolidin-1-yl)methyl)-1-isopropylquinolin-4(1H)-one

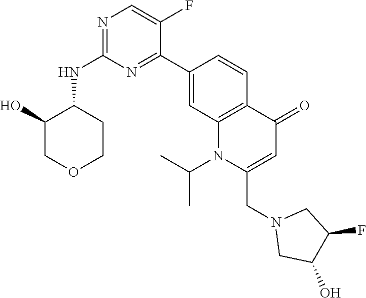 [SMILES: CC(C)n1c(CN2C[C@@H](O)[C@H](F)C2)cc(=O)c2ccc(-c3nc(N[C@@H]4CCOC[C@H]4O)ncc3F)cc21]

The title compound (15 mg, 52%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R,4R)-4-fluoropyrrolidin-3-ol hydrochloride. ¹H-NMR (400 MHz, DMSO-d6) δ 8.72 (s, 1H), 8.52 (s, 1H), 8.31 (d, J=8.4 Hz, 1H), 7.94 (d, J=8.2 Hz, 1H), 7.31 (d, J=7.9 Hz, 1H), 6.22 (s, 1H), 5.39-5.30 (m, 2H), 4.96 (d, J=4.5 Hz, 1H), 4.84 (d, J=54.3 Hz, 1H), 4.18 (d, J=25.1 Hz, 1H), 3.85-3.71 (m, 5H), 3.53 (s, 1H), 3.29 (s, 1H), 3.20-3.11 (m, 1H), 3.03 (t, J=10.3 Hz, 1H), 2.93-2.77 (m, 2H), 2.28 (s, 1H), 2.00 (s, 1H), 1.70 (t, J=7.9 Hz, 6H), 1.53-1.52 (m, 1H). LC-MS (M+H)⁺=516.1.

### Example 47: 7-(5-fluoro-2-(((3S,4R)-3-hydroxytetrahydro-2H-pyran-4-yl)amino)pyrimidin-4-yl)-1-isopropyl-2-((isopropylamino)methyl)quinolin-4(1H)-one

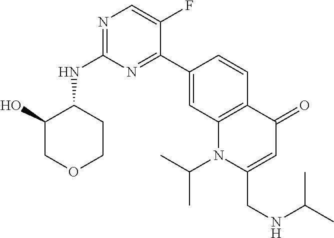 [SMILES: CC(C)NCc1cc(=O)c2ccc(-c3nc(N[C@@H]4CCOC[C@H]4O)ncc3F)cc2n1C(C)C]

The title compound (10 mg, 38%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and propan-2-amine. ¹H-NMR (400 MHz, DMSO-d6) δ 8.71 (s, 1H), 8.52 (s, 1H), 8.30 (d, J=8.5 Hz, 1H), 7.92 (d, J=8.3 Hz, 1H), 7.31 (d, J=7.9 Hz, 1H), 6.24 (s, 1H), 5.56-5.35 (m, 1H), 4.97 (s, 1H), 3.82 (s, 5H), 3.54 (s, 1H), 3.32-3.22 (m, 2H), 3.04 (t, J=10.3 Hz, 1H), 2.78 (t, J=6.1 Hz, 1H), 2.01 (d, J=10.3 Hz, 1H), 1.72 (s, 6H), 1.54-1.51 (m, 1H), 1.05 (d, J=6.0 Hz, 6H). LC-MS (M+H)⁺=470.1.

### Example 48: 2-((cyclopropylamino)methyl)-7-(5-fluoro-2-(((3S,4R)-3-hydroxytetrahydro-2H-pyran-4-yl)amino)pyrimidin-4-yl)-1-isopropylquinolin-4(1H)-one

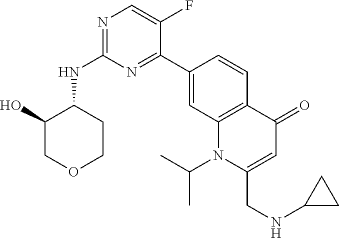 [SMILES: CC(C)n1c(CNC2CC2)cc(=O)c2ccc(-c3nc(N[C@@H]4CCOC[C@H]4O)ncc3F)cc21]

The title compound (10 mg, 38%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and cyclopropanamine. ¹H-NMR (400 MHz, DMSO-d6) δ 8.69 (s, 1H), 8.51 (s, 1H), 8.30 (d, J=8.2 Hz, 1H), 7.92 (d, J=8.2 Hz, 1H), 7.31 (d, J=8.0 Hz, 1H), 6.24 (s, 1H), 5.42-5.20 (m, 1H), 4.97 (s, 1H), 3.98-3.73 (m, 5H), 3.53 (s, 1H), 3.31-3.24 (m, 1H), 3.03 (t, J=10.3 Hz, 1H), 2.17 (s, 1H), 1.99 (s, 1H), 1.69 (t, J=6.0 Hz, 6H), 1.54-1.51 (m, 1H), 0.39 (d, J=5.4 Hz, 2H), 0.25 (s, 2H). LC-MS (M+H)⁺=468.1.

### Example 49: 7-(5-fluoro-2-(((3S,4R)-3-hydroxytetrahydro-2H-pyran-4-yl)amino)pyrimidin-4-yl)-2-(((R)-3-fluoropyrrolidin-1-yl)methyl)-1-isopropylquinolin-4(1H)-one

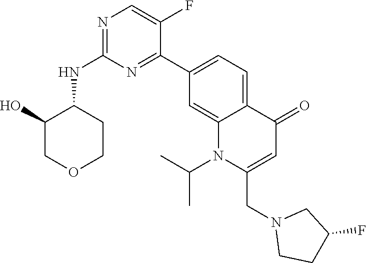 [SMILES: CC(C)n1c(CN2CC[C@@H](F)C2)cc(=O)c2ccc(-c3nc(N[C@@H]4CCOC[C@H]4O)ncc3F)cc21]

The title compound (65 mg, 25%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (R)-3-fluoropyrrolidine hydochloride. ¹H-NMR (400 MHz, DMSO-d6) δ 8.72 (s, 1H), 8.51 (d, J=3.6 Hz, 1H), 8.31 (d, J=8.5 Hz, 1H), 7.94 (d, J=8.4 Hz, 1H), 7.31 (d, J=7.9 Hz, 1H), 6.23 (s, 1H), 5.42-5.33 (m, 1H), 5.30-5.16 (m, 1H), 4.96 (d, J=4.7 Hz, 1H), 3.97-3.82 (m, 3H), 3.80-3.76 (m, 2H), 3.53 (s, 1H), 3.35-3.30 (m, 1H), 3.06-3.01 (m, 1H), 2.96-2.81 (m, 2H), 2.77-2.58 (m, 1H), 2.42-2.38 (m, 1H), 2.30-2.08 (m, 1H), 1.96-1.93 (m, 2H), 1.71 (t, J=6.4 Hz, 6H), 1.63-1.41 (m, 1H). LC-MS (M+H)⁺=500.1.

### Example 50: N-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)acetamide

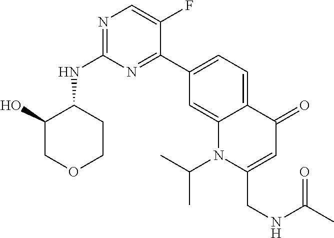 [SMILES: CC(=O)NCc1cc(=O)c2ccc(-c3nc(N[C@@H]4CCOC[C@H]4O)ncc3F)cc2n1C(C)C]

### Step 1: 2-(aminomethyl)-7-bromo-1-isopropylquinolin-4(1H)-one

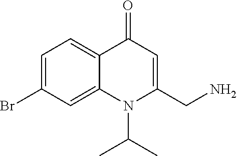 [SMILES: CC(C)n1c(CN)cc(=O)c2ccc(Br)cc21]

A mixture of 7-bromo-2-(chloromethyl)-1-isopropylquinolin-4(1H)-one (200 mg, 0.634 mmol) and ammonia (10 mL, 7N in methanol) was stirred at 40° C. for 18 h in a sealed tube before cooled to room temperature. The solvent was removed under reduced pressure, and the residue was suspended in dichloromethane (20 mL) before filtration. The filtrate was concentrated in vacuo to afford the title compound (190 mg, 99%). LC-MS (M+H)⁺=295.

### Step 2: tert-butyl ((7-bromo-1-isopropyl-4-oxo-1,4-dihydroquinolin-2-yl)methyl)carbamate

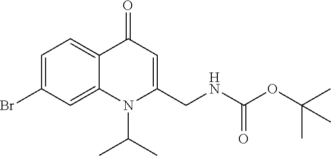 [SMILES: CC(C)n1c(CNC(=O)OC(C)(C)C)cc(=O)c2ccc(Br)cc21]

A solution of 2-(aminomethyl)-7-bromo-1-isopropylquinolin-4(1H)-one (190 mg, 0.644 mmol), di-tert-butyl dicarbonate (327 mg, 0.966 mmol) and triethylamine (194 mg, 1.92 mmol) in dichloromethane (15 mL) was stirred at 25° C. for 18 h. The solvent was removed under reduced pressure. The residue was purified over silica gel by combi-flash, eluting with methanol in dichloromethane (5% gradient, v/v) to give the title compound (190 mg, 75%). LC-MS (M+H)⁺=395.

### Step 3: tert-butyl ((1-isopropyl-4-oxo-7-(4,4,5,5-tetramethyl-1,3,2-dioxaborolan-2-yl)-1,4-dihydroquinolin-2-yl)methyl)carbamate

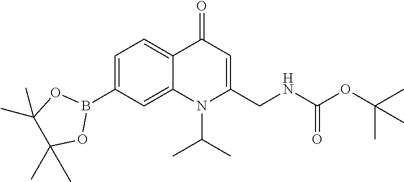 [SMILES: CC(C)n1c(CNC(=O)OC(C)(C)C)cc(=O)c2ccc(B3OC(C)(C)C(C)(C)O3)cc21]

The title compound (213 mg, 100%) was prepared in a manner similar to Example 1 step 6 from tert-butyl ((7-bromo-1-isopropyl-4-oxo-1,4-dihydroquinolin-2-yl)methyl)carbamate and bis(pinacolato)diboron. LC-MS (M+H)⁺=443.

### Step 4: tert-butyl ((7-(2-chloro-5-fluoropyrimidin-4-yl)-1-isopropyl-4-oxo-1,4-dihydroquinolin-2-yl)methyl)carbamate

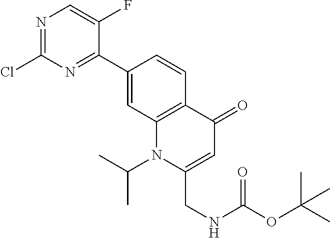 [SMILES: CC(C)n1c(CNC(=O)OC(C)(C)C)cc(=O)c2ccc(-c3nc(Cl)ncc3F)cc21]

The title compound (190 mg, 88%) was prepared in a manner similar to Example 1 step 7 from tert-butyl ((1-isopropyl-4-oxo-7-(4,4,5,5-tetramethyl-1,3,2-dioxaborolan-2-yl)-1,4-dihydroquinolin-2-yl)methyl)carbamate and 2,4-dichloro-5-fluoropyrimidine. LC-MS (M+H)⁺=447.0.

### Step 5: 2-(aminomethyl)-7-(2-chloro-5-fluoropyrimidin-4-yl)-1-isopropylquinolin-4(1H)-one

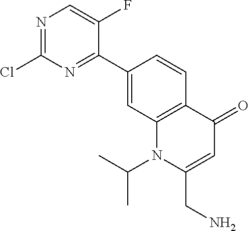 [SMILES: CC(C)n1c(CN)cc(=O)c2ccc(-c3nc(Cl)ncc3F)cc21]

A solution of tert-butyl ((7-(2-chloro-5-fluoropyrimidin-4-yl)-1-isopropyl-4-oxo-1,4-dihydroquinolin-2-yl)methyl)carbamate (190 mg, 0.55 mmol) in trifluoroacetic acid (2 mL) and dichloromethane (6 mL) was stirred at 25° C. for 3 h. The solvents were removed under reduced pressure. The residue was dissolved in dichloromethane (10 mL) and aqueous saturated sodium bicarbonate solution (2 mL) was added. The aqueous layer was extracted with dichloromethane (10 mL×2). The combined organic layers were dried over sodium sulfate, filtered and concentrated. The residue was purified over silica gel by combi-flash, eluting with methanol in dichloromethane (10%, v/v) to give the title compound (120 mg, 89%). LC-MS (M+H)⁺=347.

### Step 6: N-((7-(2-chloro-5-fluoropyrimidin-4-yl)-1-isopropyl-4-oxo-1,4-dihydroquinolin-2-yl)methyl)acetamide

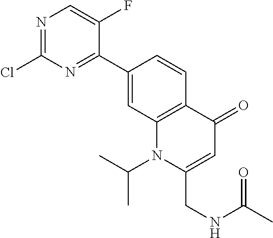 [SMILES: CC(=O)NCc1cc(=O)c2ccc(-c3nc(Cl)ncc3F)cc2n1C(C)C]

To a solution of 2-(aminomethyl)-7-(2-chloro-5-fluoropyrimidin-4-yl)-1-isopropylquinolin-4(1H)-one (112 mg, 0.346 mmol) in tetrahydrofuran (5 mL) were added pyridine (55 mg, 0.69 mmol) followed by acetic anhydride (53 mg, 0.52 mmol). The reaction was stirred at 25° C. for 1 h. The solvent was removed under reduced pressure. Aqueous saturated solution of sodium bicarbonate (2 mL) was added and the aqueous solution was extracted with dichloromethane (10 mL×3). The combined organic layers were dried over sodium sulfate, filtered and concentrated. The residue was purified over silica gel by combi-flash, eluting with methanol in dichloromethane (10%, v/v) to afford the tittle compound (112 mg, 83%). LC-MS (M+H)⁺=389.

### Step 7: N-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)acetamide

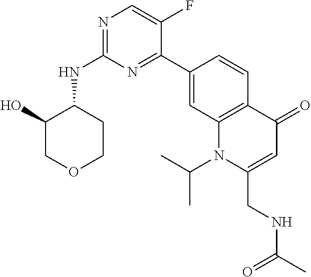 [SMILES: CC(=O)NCc1cc(=O)c2ccc(-c3nc(N[C@@H]4CCOC[C@H]4O)ncc3F)cc2n1C(C)C]

The title compound (23 mg, 32%) was prepared in a manner similar to Example 1 step 8 from N-((7-(2-chloro-5-fluoropyrimidin-4-yl)-1-isopropyl-4-oxo-1,4-dihydroquinolin-2-yl)methyl)acetamide and (3S,4R)-4-aminotetrahydro-2H-pyran-3-ol. ¹H-NMR (400 MHz, DMSO-d6) δ: 8.69 (s, 1H), 8.57 (s, 1H), 8.52 (s, 1H), 8.30 (d, J=8.2 Hz, 1H), 7.94 (d, J=7.8 Hz, 1H), 7.32 (d, J=7.7 Hz, 1H), 6.19 (s, 1H), 5.04-4.84 (m, 2H), 4.48 (s, 2H), 3.97-3.77 (m, 3H), 3.53 (s, 1H), 3.30 (s, 1H), 3.04 (t, J=10.2 Hz, 1H). 2.01 (d, J=13.3 Hz, 1H), 1.94 (s, 3H), 1.70 (s, 6H), 1.52 (q, J=12.9 Hz. 1H). LC-MS (M+H)⁺=470.

### Example 51 and Example 52: (R)-7-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)-2-methyl-2,7-diazaspiro[4,5]decan-1-one and (S)-7-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)-2-methyl-2,7-diazaspiro[4,5]decan-1-one

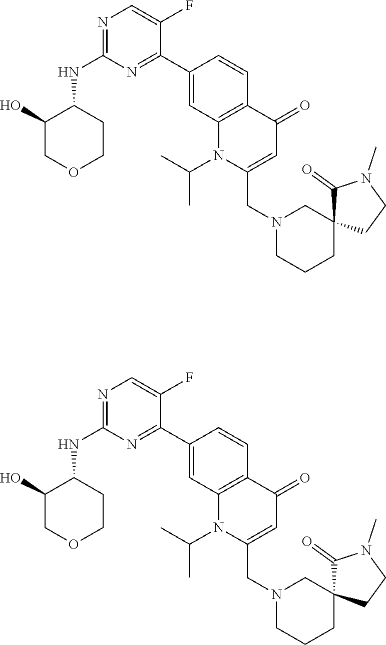 [SMILES: CC(C)n1c(CN2CCC[C@@]3(CCN(C)C3=O)C2)cc(=O)c2ccc(-c3nc(N[C@@H]4CCOC[C@H]4O)ncc3F)cc21 ; CC(C)n1c(CN2CCC[C@]3(CCN(C)C3=O)C2)cc(=O)c2ccc(-c3nc(N[C@@H]4CCOC[C@H]4O)ncc3F)cc21]

Mixture of Example 51 and Example 52 was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and racemic 2-methyl-2,7-diazaspiro[4.5]decan-1-one. Example 51 and Example 52 were separated by chiral prep-HPLC using 50% mobile phase A and 50% mobile phase B. Chiral HPLC condition: Cellulose-C column, 20.0 mm×250 mm, 5 um. Mobile phase A: hexane, Mobile phase B: ethanol containing 0.2% 2M NH₃ in methanol, 18 mL/min in 13 min.

Example 51 (12 mg, 60%). RT=6.5 min. ¹H-NMR (400 MHz, DMSO-d6) δ 8.72 (s, 1H), 8.52 (s, 1H), 8.30 (d, J=8.3 Hz, 1H), 7.93 (d, J=7.9 Hz, 1H), 7.31 (d, J=7.8 Hz, 1H), 6.15 (s, 1H), 5.41 (m, 1H), 4.97 (s, 1H), 3.86 (m, 3H), 3.66-3.63 (m, 1H), 3.51-3.48 (m, 2H), 3.32-3.29 (m, 1H), 3.23 (m, 2H), 3.04 (m, 1H), 2.81 (m, 1H), 2.70 (s, 3H), 2.63-2.60 (m, 1H), 2.17-2.14 (m, 1H), 2.12-1.93 (m, 3H), 1.90 (brs, 1H), 1.73 (s, 6H), 1.64-1.34 (m, 5H). LC-MS (M+H)⁺=579.1.

Example 52 (12 mg, 60%). RT=10.5 min. ¹H-NMR (400 MHz, DMSO-d6) δ 8.73 (s, 1H), 8.52 (s, 1H), 8.30 (d, J=8.1 Hz, 1H), 7.94 (d, J=8.2 Hz, 1H), 7.32 (d, J=7.5 Hz, 1H), 6.16 (s, 1H), 5.41 (m, 1H), 4.97 (s, 1H), 3.84 (m, 3H), 3.66-3.63 (m, 1H), 3.51-3.47 (m, 2H), 3.30 (s, 1H), 3.24 (m, 2H), 3.04 (t, J=10.7 Hz, 1H), 2.83 (d, J=10.0 Hz, 1H), 2.70 (s, 3H), 2.62 (d. J=10.7 Hz, 1H), 2.16 (d, J=10.9 Hz, 1H), 2.10-1.84 (m, 4H), 1.73 (t, J=6.6 Hz, 6H), 1.66-1.33 (m, 5H). LC-MS (M+H)⁺=579.1.

### Example 53: 7-(5-fluoro-2-(((3S,4R)-3-hydroxytetrahydro-2H-pyran-4-yl)amino)pyrimidin-4-yl)-1-isopropyl-2-(((S)-3-methylmorpholino)methyl)quinolin-4(1H)-one

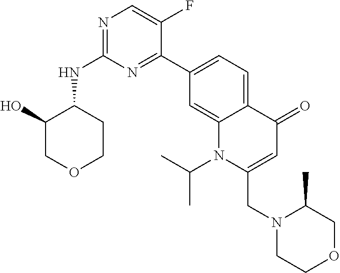 [SMILES: CC(C)n1c(CN2CCOC[C@@H]2C)cc(=O)c2ccc(-c3nc(N[C@@H]4CCOC[C@H]4O)ncc3F)cc21]

The title compound (12 mg, 33%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S)-3-methylmorpholine. ¹H-NMR (300 MHz, DMSO-d6) δ 8.73 (brs, 1H), 8.52 (d, J=3.8 Hz, 1H), 8.30 (d, J=8.4 Hz, 1H), 7.94 (d, J=8.4 Hz, 1H), 7.31 (d, J=8.0 Hz, 1H), 6.22 (s, 1H), 5.48 (s, 1H), 4.97 (d, J=5.1 Hz, 1H), 4.32-4.22 (m, 1H), 3.99-3.79 (m, 3H), 3.72-3.59 (m, 2H), 3.55-3.44 (m, 2H), 3.38-3.34 (m, 1H), 3.27-3.17 (m, 2H), 3.11-2.98 (m, 1H), 2.67-2.51 (m, 2H), 2.29-2.23 (m, 1H), 2.07-1.96 (m, 1H), 1.79-1.69 (m, 6H), 1.58-1.47 (m, 1H), 1.08 (d, J=6.2 Hz, 3H). LC-MS (M+H)⁺=512.2.

### Example 54: 2-((4,4-difluoropiperidin-1-yl)methyl)-7-(5-fluoro-2-(((3S,4R)-3-hydroxytetrahydro-2H-pyran-4-yl)<amino)pyrimidin-4-yl)-1-isopropylquinolin-4(1H)-one

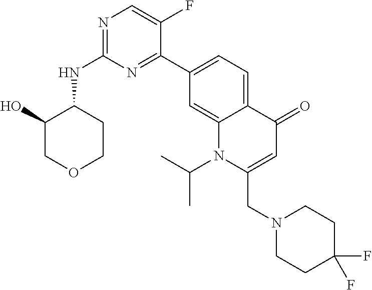 [SMILES: CC(C)n1c(CN2CCC(F)(F)CC2)cc(=O)c2ccc(-c3nc(N[C@@H]4CCOC[C@H]4O)ncc3F)cc21]

The title compound (14 mg, 35%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4,4-difluoropiperidine. ¹H-NMR (400 MHz, DMSO-d6) δ 8.76 (brs, 1H), 8.52 (d, J=3.8 Hz, 1H), 8.31 (d, J=8.4 Hz, 1H), 7.98-7.91 (m, 1H), 7.33 (d, J=8.0 Hz, 1H), 6.20 (s, 1H), 5.39-5.28 (m, 1H), 4.98 (d, J=5.2 Hz, 1H), 3.97-3.81 (m, 3H), 3.72 (s, 2H), 3.60-3.48 (m, 1H), 3.39-3.28 (m, 11H), 3.09-2.99 (m, 1H), 2.63-2.58 (m, 4H), 2.03-1.93 (m, 5H), 1.78-1.70 (m, 6H), 1.60-1.45 (m, 1H). LC-MS (M+H)⁺=532.3.

### Example 55: 2-ethyl-7-(5-fluoro-2-(((3S,4R)-3-hydroxytetrahydro-2H-pyran-4-yl)amino)pyrimidin-4-yl)-1-isopropylquinolin-4(1H)-one

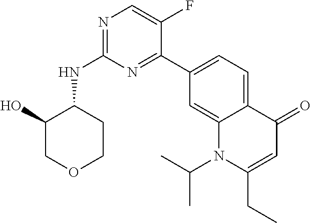 [SMILES: CCc1cc(=O)c2ccc(-c3nc(N[C@@H]4CCOC[C@H]4O)ncc3F)cc2n1C(C)C]

### Step 1: N-(2-acetyl-5-bromophenyl)-N-isopropylpropionamide

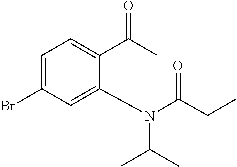 [SMILES: CCC(=O)N(c1cc(Br)ccc1C(C)=O)C(C)C]

To a solution of 1-(4-bromo-2-(isopropylamino)phenyl)ethan-1-one (2.4 g, 9.392 mmol) in tetrahydrofuran (40.0 mL) was added potassium bis(trimethylsilyl)amide (18.78 mL, 18.78 mmol, 1M in tetrahydrofuran) at 0° C. under nitrogen atmosphere. The resulting mixture was stirred for 30 min at 0° C. under nitrogen atmosphere. To the above mixture was added propanoyl chloride (1.30 g, 14.09 mmol) at 0° C. The resulting mixture was stirred for 5 h at room temperature under nitrogen atmosphere before quenched by addition of water (20 mL). The resulting solution was extracted with ethyl acetate (20 mL×3). The organic phases were combined, washed with brine, dried over sodium sulfate, filtered and concentrated. The residue was purified by flash chromatography eluting with ethyl acetate in petroleum ether (0% to 25% gradient, v/v) to yield the title compound (1.0 g, 34%). LC-MS (M+H)⁺=312.0.

### Step 2: 7-bromo-2-ethyl-1-isopropylquinolin-4(1H)-one

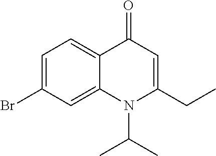 [SMILES: CCc1cc(=O)c2ccc(Br)cc2n1C(C)C]

The title compound (166 mg, 17%) was prepared in a manner similar to that in Example 7 step 3 from N-(2-acetyl-5-bromophenyl)-N-isopropylpropionamide. LC-MS (M+H)⁺=294.1.

### Step 3: 2-ethyl-1-isopropyl-7-(4,4,5,5-tetramethyl-1,3,2-dioxaborolan-2-yl)quinolin-4(1H)-one

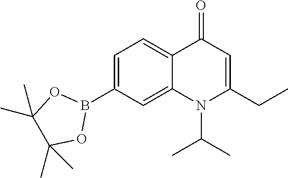 [SMILES: CCc1cc(=O)c2ccc(B3OC(C)(C)C(C)(C)O3)cc2n1C(C)C]

The title compound (100 mg, 51%) was prepared in a manner similar to that in Example 1 step 6 from 7-bromo-2-ethyl-1-isopropylquinolin-4(1H)-one. LC-MS (M+H)⁺=260.1 for corresponding boronic acid.

### Step 4: 2-chloro-5-fluoro-4-((4-methoxybenzyl)oxy)pyrimidine

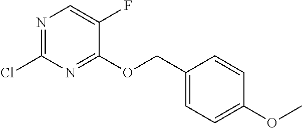 [SMILES: COc1ccc(COc2nc(Cl)ncc2F)cc1]

To a solution of sodium hydride (925 mg, 23.13 mmol, 60%) in tetrahydrofuran (30 mL) was added (4-methoxyphenyl)methanol (2350 mg, 17.018 mmol) in tetrahydrofuran (12.0 mL) dropwise at 0° C. under nitrogen atmosphere. The resulting mixture was stirred for 30 min at 0° C. under nitrogen atmosphere. To the above mixture was added 2,4-dichloro-5-fluoropyrimidine (2375 mg, 14.23 mmol) in tetrahydrofuran (12 mL) dropwise over 20 min at 0° C. The resulting mixture was stirred for additional 2 h at 0° C. under nitrogen atmosphere before quenched by the addition of saturated aqueous ammonium chloride solution (30 mL). The resulting solution was extracted with ethyl acetate (40 mL×2). The organic phases were combined, washed with brine, dried over sodium sulfate, filtered and concentrated. The residue was purified by flash chromatography eluting with ethyl acetate in petroleum ether (0% to 10% gradient, v/v) to yield the title compound (3300 mg, 86%). LC-MS (M+H)⁺=269.2.

### Step 5: (3S,4R)-4-((5-fluoro-4-((4-methoxybenzyl)oxy)pyrimidin-2-yl)amino)tetrahydro-2H-pyran-3-ol

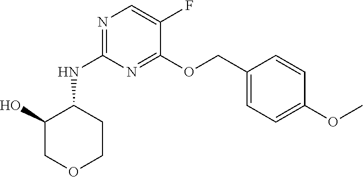 [SMILES: COc1ccc(COc2nc(N[C@@H]3CCOC[C@H]3O)ncc2F)cc1]

The title compound (1900 mg, 73%) was prepared in a manner similar to that in Example 2 step 4 from 2-chloro-5-fluoro-4-((4-methoxybenzyl)oxy)pyrimidine and (3S,4R)-4-aminooxan-3-ol hydrochloride. LC-MS (M+H)⁺=350.1.

### Step 6: (3S,4R)-4-(5-fluoro-4-((4-methoxybenzyl)oxy)pyrimidin-2-yl)amino)tetrahydro-2H-pyran-3-yl acetate

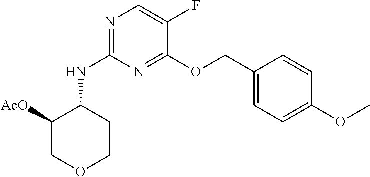 [SMILES: COc1ccc(COc2nc(N[C@@H]3CCOC[C@H]3OC(C)=O)ncc2F)cc1]

To a solution of (3S,4R)-4-((5-fluoro-4-((4-methoxybenzyl)oxy)pyrimidin-2-yl)amino)tetrahydro-2H-pyran-3-ol (1900 mg, 5.43 mmol) and triethylamine (4400 mg, 43.47 mmol) in dichloromethane (40 mL) was added acetic anhydride (3325 mg, 32.57 mmol) dropwise at 0° C. under nitrogen atmosphere. The resulting mixture was stirred for 16 h at room temperature under nitrogen atmosphere before quenched by the addition of aqueous saturated sodium bicarbonate solution (30 mL). The resulting mixture was extracted with dichloromethane (40 mL×2). The organic phases were combined, washed with brine, dried over sodium sulfate, filtered and concentrated. The residue was purified by flash chromatography eluting with ethyl acetate in dichloromethane (0% to 25% gradient, v/v) to yield the title compound (1950 mg, 91%). LC-MS (M+H)⁺=392.0.

### Step 7: (3S,4R)-4-((5-fluoro-4-hydroxypyrimidin-2-yl)amino)tetrahydro-2H-pyran-3-yl acetate

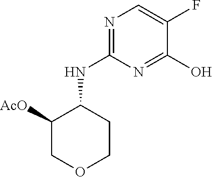 [SMILES: CC(=O)O[C@@H]1COCC[C@H]1Nc1ncc(F)c(O)n1]

To a solution of (3S,4R)-4-((5-fluoro-4-((4-methoxybenzyl)oxy)pyrimidin-2-yl)amino)tetrahydro-2H-pyran-3-yl acetate (1950 mg, 4.98 mmol) in methanol (30 mL) was added Pd/C (945 mg, 0.888 mmol, 10%) under nitrogen atmosphere. The mixture was hydrogenated at room temperature for 6 h under 1 atmosphere of hydrogen. The reaction was filtered through a Celite pad and concentrated under reduced pressure to yield the title compound (1200 mg, 88%). LC-MS (M+H)⁺=272.0.

### Step 8: (3S,4R)-4-((4-chloro-5-fluoropyrimidin-2-yl)amino)tetrahydro-2H-pyran-3-yl acetate

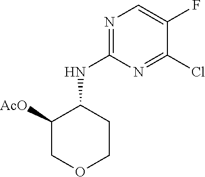 [SMILES: CC(=O)O[C@@H]1COCC[C@H]1Nc1ncc(F)c(Cl)n1]

To a solution of (3S,4R)-4-((5-fluoro-4-hydroxypyrimidin-2-yl)amino)tetrahydro-2H-pyran-3-yl acetate (1200 mg, 4.42 mmol) and N,N-diethylaniline (1985 mg, 13.31 mmol) in 1,2-dichloroethane (25 mL) was added phosphoryl chloride (2055 mg, 13.42 mmol) dropwise at room temperature under nitrogen atmosphere. The resulting mixture was stirred for 3 h at 100° C. under nitrogen atmosphere before cooled to room temperature and concentrated. The residue was purified by flash chromatography eluting with ethyl acetate in petroleum ether (0% to 30% gradient) to yield the title compound (820 mg, 63%). LC-MS (M+H)⁺=290.0.

### Step 9: (3S,4R)-4-(4-(2-ethyl-1-isopropyl-4-oxo-1,4-dihydroquinolin-7-yl)-5-fluoropyrimidin-2-yl)amino)tetrahydro-2H-pyran-3-yl acetate

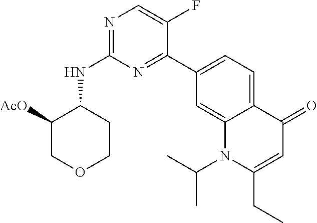 [SMILES: CCc1cc(=O)c2ccc(-c3nc(N[C@@H]4CCOC[C@H]4OC(C)=O)ncc3F)cc2n1C(C)C]

The title compound (75 mg, 66%) was prepared in a manner similar to that in Example 1 step 7 from (3S,4R)-4-((4-chloro-5-fluoropyrimidin-2-yl)amino)tetrahydro-2H-pyran-3-yl acetate and 2-ethyl-1-isopropyl-7-(4,4,5,5-tetramethyl-1,3,2-dioxaborolan-2-yl)quinolin-4(1H)-one. LC-MS (M+H)⁺=469.3.

### Step 10: 2-ethyl-7-(5-fluoro-2-(((3S,4R)-3-hydroxytetrahydro-2H-pyran-4-yl)amino)pyrimidin-4-yl)-1-isopropylquinolin-4(1H)-one

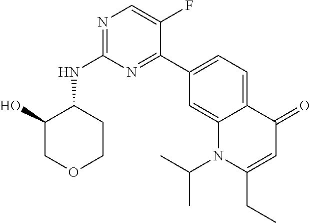 [SMILES: CCc1cc(=O)c2ccc(-c3nc(N[C@@H]4CCOC[C@H]4O)ncc3F)cc2n1C(C)C]

A solution of (3S,4R)-4-((4-(2-ethyl-1-isopropyl-4-oxo-1,4-dihydroquinolin-7-yl)-5-fluoropyrimidin-2-yl)amino)tetrahydro-2H-pyran-3-yl acetate (75 mg, 0.16 mmol) and hydrogen chloride in methanol (3.0 mL, 4 M) was stirred for 2 h at 60° C. under nitrogen atmosphere before cooled to room temperature and concentrated. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₃·H₂O). 20% to 48% gradient in 9 min; detector, UV 254 nm. The title compound (39 mg, 57%) was obtained. ¹H NMR (300 MHz, DMSO-d₆) δ 8.69 (brs, 1H), 8.51 (d, J=3.8 Hz, 1H), 8.30 (d, J=8.4 Hz, 11H), 7.98-7.88 (m, 1H), 7.30 (d, J=8.0 Hz, 1H), 6.11 (s, 1H), 5.18-5.03 (m, 1H), 4.97 (d, J=5.2 Hz, 1H), 3.91-3.80 (m, 3H), 3.62-3.46 (m, 1H), 3.40-3.26 (m, 1H), 3.11-2.98 (m, 1H), 2.93-2.79 (m, 2H), 2.07-1.97 (m, 1H), 1.78-1.63 (m, 6H). 1.63-1.43 (m, 1H), 1.29 (t, J=7.4 Hz, 3H). LC-MS (M+H)⁺=427.1.

### Example 56: 7-(5-fluoro-2-(((3S,4R)-3-hydroxytetrahydro-2H-pyran-4-yl)amino)pyrimidin-4-yl)-2-(((S)-3-hydroxypiperidin-1-yl)methyl)-1-isopropylquinolin-4(1H)-one

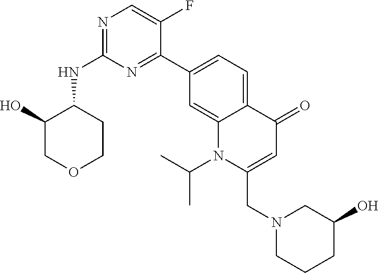 [SMILES: CC(C)n1c(CN2CCC[C@H](O)C2)cc(=O)c2ccc(-c3nc(N[C@@H]4CCOC[C@H]4O)ncc3F)cc21]

The title compound (16 mg, 47%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S)-piperidin-3-ol hydrochloride. ¹H-NMR (400 MHz, DMSO-d6) δ 8.74 (brs, 1H), 8.52 (d, J=3.8 Hz, 1H), 8.31 (d, J=8.4 Hz, 1H), 7.94 (d, J=8.4 Hz, 1H), 7.33 (d, J=8.0 Hz, 1H), 6.17 (s, 1H), 5.40-5.28 (m, 1H), 4.98 (d, J=5.2 Hz, 1H), 4.65 (d, J=4.5 Hz, 1H), 3.99-3.80 (m, 3H), 3.73-3.65 (m, 1H), 3.60-3.42 (m, 3H), 3.38-3.28 (m, 1H), 3.09-2.99 (m, 1H), 2.89-2.81 (m, 1H), 2.75-2.67 (m, 1H), 2.03-1.93 (m, 2H), 1.89-1.77 (m, 2H), 1.75-1.61 (m, 7H), 1.60-1.36 (m, 2H), 1.17-1.04 (m, 1H). LC-MS (M+H)⁺=512.2.

### Example 57: 7-(5-fluoro-2-(((3S,4R)-3-hydroxytetrahydro-2H-pyran-4-yl)amino)pyrimidin-4-yl)-2-(((R)-3-hydroxypiperidin-1-yl)methyl)-1-isopropylquinolin-4(1H)-one

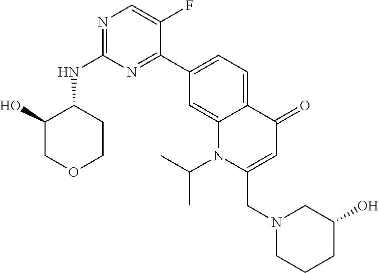 [SMILES: CC(C)n1c(CN2CCC[C@@H](O)C2)cc(=O)c2ccc(-c3nc(N[C@@H]4CCOC[C@H]4O)ncc3F)cc21]

The title compound (11 mg, 32%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R)-piperidin-3-ol hydrochloride. ¹H-NMR (400 MHz, DMSO-d6) δ 8.74 (brs, 1H), 8.52 (d, J=3.8 Hz, 1H), 8.31 (d, J=8.4 Hz, 1H), 7.94 (d, J=8.4 Hz, 1H), 7.32 (d, J=8.0 Hz, 1H), 6.17 (s, 1H), 5.40-5.28 (m, 1H), 4.98 (d, J=5.2 Hz, 1H), 4.65 (d, J=4.6 Hz, 1H), 3.92-3.80 (m, 3H), 3.73-3.65 (m, 1H), 3.60-3.41 (m, 3H), 3.39-3.28 (m, 1H), 3.09-2.99 (m, 1H), 2.89-2.81 (m, 1H), 2.74-2.67 (m, 1H), 2.03-1.94 (m, 2H), 1.89-1.76 (m, 2H), 1.76-1.61 (m, 7H), 1.57-1.34 (m, 2H), 1.17-1.04 (m, 1H). LC-MS (M+H)⁺=512.3.

### Example 58: 7-(5-fluoro-2-(((3S,4R)-3-hydroxytetrahydro-2H-pyran-4-yl)amino)pyrimidin-4-yl)-1-isopropyl-3-methyl-2-(morpholinomethyl)quinolin-4(1H)-one

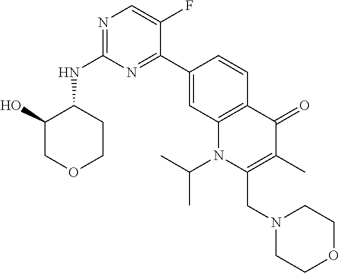 [SMILES: Cc1c(CN2CCOCC2)n(C(C)C)c2cc(-c3nc(N[C@@H]4CCOC[C@H]4O)ncc3F)ccc2c1=O]

### Step 1: 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

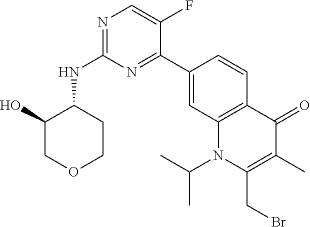 [SMILES: Cc1c(CBr)n(C(C)C)c2cc(-c3nc(N[C@@H]4CCOC[C@H]4O)ncc3F)ccc2c1=O]

To a mixture of 7-(5-fluoro-2-(((3S,4R)-3-hydroxytetrahydro-2H-pyran-4-yl)amino)pyrimidin-4-yl)-1-isopropyl-2,3-dimethylquinolin-4(1H)-one (640 mg, 1.5 mmol) and N-bromosuccinimide (218 mg, 1.2 mmol) in carbon tetrachloride (25 mL) was added azobisisobutyronitrile (46 mg, 0.3 mmol). The mixture was stirred at 70° C. for 2 h before cooled to room temperature and concentrated. The residue was purified over silica gel by combi-flash, eluting with methanol in dichloromethane (3%, v/v) to give the title compound (300 mg, 39%). LC-MS (M+H)⁺=505.1, 507.1.

### Step 2: 7-(5-fluoro-2-(((3S,4R)-3-hydroxytetrahydro-2H-pyran-4-yl)amino)pyrimidin-4-yl)-1-isopropyl-3-methyl-2-(morpholinomethyl)quinolin-4(1H)-one

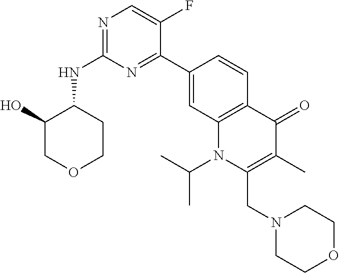 [SMILES: Cc1c(CN2CCOCC2)n(C(C)C)c2cc(-c3nc(N[C@@H]4CCOC[C@H]4O)ncc3F)ccc2c1=O]

The title compound (20 mg, 39%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and morpholine. ¹H-NMR (400 MHz, DMSO-d6) δ 8.74 (brs, 1H), 8.51 (s, 1H), 8.34 (d, J=8.3 Hz, 1H), 7.90 (d, J=8.9 Hz, 1H), 7.30 (d, J=7.7 Hz, 1H), 5.53-5.43 (m, 1H), 4.97 (s, 1H), 3.92-2.72 (m, 5H), 3.67-3.47 (m, 5H), 3.38-3.28 (m, 1H), 3.04 (t, J=10.6 Hz, 1H), 2.60-2.48 (m, 4H), 2.18 (s, 3H), 2.05-1.95 (m, 1H), 1.80-1.70 (m, 6H), 1.58-1.48 (m, 1H). LC-MS (M+H)⁺=512.1.

### Example 59: 7-(5-fluoro-2-(((3S,4R)-3-hydroxytetrahydro-2H-pyran-4-yl)amino)pyrimidin-4-yl)-1-isopropyl-2-((((R)-tetrahydro-2H-pyran-3-yl)amino)methyl)quinolin-4(1H)-one

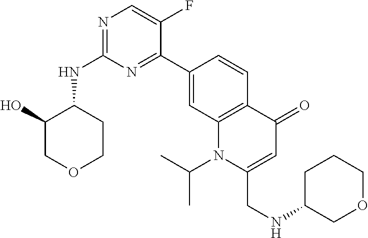 [SMILES: CC(C)n1c(CN[C@@H]2CCCOC2)cc(=O)c2ccc(-c3nc(N[C@@H]4CCOC[C@H]4O)ncc3F)cc21]

The title compound (8 mg, 30%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (R)-tetrahydro-2H-pyran-3-amine. ¹H-NMR (400 MHz, DMSO-d6) δ 8.70 (brs, 1H), 8.51 (d, J=3.0 Hz, 1H), 8.30 (d, J=8.4 Hz, 1H), 7.93 (d, J=9.1 Hz, 1H), 7.31 (d, J=7.8 Hz, 1H), 6.25 (s, 1H), 5.43-5.33 (m, 1H), 4.98 (s, 1H), 3.95-3.80 (m, 6H), 3.72-3.65 (m, 1H), 3.58-3.48 (m, 1H), 3.35-3.25 (m, 2H), 3.10-3.00 (m, 2H), 2.65-2.50 (m, 1H), 2.05-1.92 (m, 2H), 1.80-1.60 (m, 8H), 1.58-1.40 (m, 2H), 1.36-1.24 (m, 1H). LC-MS (M+H)⁺=512.1.

### Example 60: 7-(5-fluoro-2-(((3S,4R)-3-hydroxytetrahydro-2H-pyran-4-yl)amino)pyrimidin-4-yl)-1-isopropyl-2-((((S)-tetrahydro-2H-pyran-3-yl)amino)methyl)quinolin-4(1H)-one

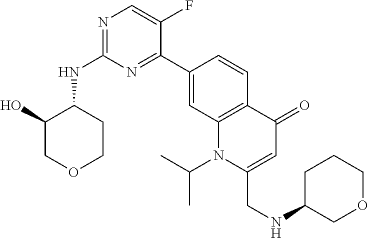 [SMILES: CC(C)n1c(CN[C@H]2CCCOC2)cc(=O)c2ccc(-c3nc(N[C@@H]4CCOC[C@H]4O)ncc3F)cc21]

The title compound (6 mg, 23%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-tetrahydro-2H-pyran-3-amine. ¹H-NMR (400 MHz, DMSO-d6) δ 8.70 (brs, 1H), 8.51 (d, J=3.0 Hz, 1H), 8.30 (d, J=8.4 Hz, 1H), 7.93 (d, J=9.1 Hz, 1H), 7.31 (d, J=7.8 Hz, 1H), 6.25 (s, 1H), 5.43-5.33 (m, 1H), 4.98 (s, 1H), 3.95-3.80 (m, 6H), 3.72-3.65 (m, 1H), 3.58-3.48 (m, 1H), 3.35-3.25 (m, 2H), 3.10-3.00 (m, 2H), 2.65-2.50 (m, 1H), 2.05-1.92 (m, 2H), 1.80-1.60 (m, 8H), 1.58-1.40 (m, 2H), 1.36-1.24 (m, 1H). LC-MS (M+H)⁺=512.1.

### Example 61: 7-(5-fluoro-2-(((3S,4R)-3-hydroxytetrahydro-2H-pyran-4-yl)amino)pyrimidin-4-yl)-2-(((S)-3-hydroxy-3-methylpyrrolidin-1-yl)methyl)-1-isopropylquinolin-4(1H)-one

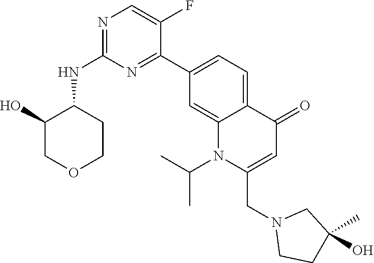 [SMILES: CC(C)n1c(CN2CC[C@](C)(O)C2)cc(=O)c2ccc(-c3nc(N[C@@H]4CCOC[C@H]4O)ncc3F)cc21]

The title compound (6 mg, 26%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-3-methylpyrrolidin-3-ol. ¹H-NMR (400 MHz, DMSO-d6) δ 8.72 (s, 1H), 8.52 (d, J=3.0 Hz, 1H), 8.30 (d, J=8.3 Hz, 1H), 7.94 (d, J=7.7 Hz, 1H), 7.31 (d, J=7.6 Hz, 1H), 6.20 (s, 1H), 5.41 (t, 1H), 4.97 (s, 1H), 4.59 (s, 1H), 3.86-3.80 (m, 3H), 3.72 (dd, J=35.3, 13.4 Hz, 2H), 3.53-3.41 (m, 1H), 3.31-3.27 (m, 1H), 3.04 (t, J=10.8 Hz, 1H), 2.71-2.61 (m, 2H), 2.66-2.45 (m, 2H), 2.00 (d, 1H), 1.76-1.60 (m, 8H), 1.53-1.48 (m, 1H), 1.26 (s, 3H). LC-MS (M+H)⁺=512.1.

### Example 62: 7-(5-fluoro-2-(((3S,4R)-3-hydroxytetrahydro-2H-pyran-4-yl)amino)pyrimidin-4-yl)-1-isopropyl-2-(((S)-2-methylmorpholino)methyl)quinolin-4(1H)-one

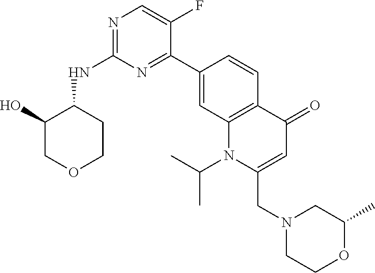 [SMILES: CC(C)n1c(CN2CCO[C@@H](C)C2)cc(=O)c2ccc(-c3nc(N[C@@H]4CCOC[C@H]4O)ncc3F)cc21]

The title compound (13 mg, 45%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-2-methylmorpholine. ¹H-NMR (400 MHz, DMSO-d6) δ 8.74 (s, 1H), 8.52 (d, J=3.0 Hz, 1H), 8.30 (d, J=8.5 Hz, 1H), 7.94 (d, J=8.4 Hz, 1H), 7.32 (d, J=7.7 Hz, 1H), 6.18 (s, 1H), 5.47-5.30 (m, 1H), 4.98 (s, 1H), 3.96-3.76 (m, 3H), 3.78-3.76 (m, 1H), 3.69-3.58 (m, 2H), 3.57-3.42 (m, 3H), 3.41-3.26 (m, 1H), 3.04 (t, J=10.5 Hz, 1H), 2.76 (d, J=11.1 Hz, 1H), 2.69 (d, J=10.6 Hz, 1H), 2.16 (t, J=10.8 Hz, 1H), 2.01 (d, J=11.1 Hz, 1H), 1.86 (t, J=10.2 Hz, 11H), 1.75-1.72 (m, 6H), 1.54-1.51 (m, 1H), 1.05 (d, J=5.8 Hz, 3H). LC-MS (M+H)⁺=513.1.

### Example 63: 7-(5-fluoro-2-(((3S,4R)-3-hydroxytetrahydro-2H-pyran-4-yl)amino)pyrimidin-4-yl)-2-((((1r,3R)-3-fluorocyclobutyl)amino)methyl)-1-isopropylquinolin-4(1H)-one

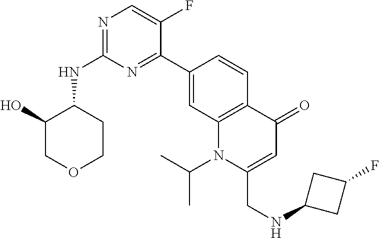 [SMILES: CC(C)n1c(CN[C@H]2C[C@H](F)C2)cc(=O)c2ccc(-c3nc(N[C@@H]4CCOC[C@H]4O)ncc3F)cc21]

The title compound (12 mg, 43%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (1r,3r)-3-fluorocyclobutan-1-amine. ¹H-NMR (400 MHz, DMSO-d6) δ 8.71 (s, 1H), 8.52 (d, J=3.0 Hz, 1H), 8.30 (d, J=8.2 Hz, 1H), 7.93 (d, J=8.3 Hz, 1H), 7.31 (d, J=7.7 Hz, 1H), 6.23 (s, 1H), 5.37-5.33 (m, 1H), 5.28-5.14 (m, 1H), 4.98 (s, 1H), 3.86-3.64 (m, 5H), 3.54-3.46 (m, 2H), 3.40-3.20 (m, 1H), 3.04 (t, J=10.1 Hz, 1H), 2.31-2.28 (m, 2H), 2.22-2.18 (m, 2H), 2.02 (d, J=11.9 Hz, 1H), 1.75-1.72 (m, 6H), 1.54-1.51 (m, 1H). LC-MS (M+H)⁺=500.1.

### Example 64: 7-(5-fluoro-2-(((3S,4R)-3-hydroxytetrahydro-2H-pyran-4-yl)amino)pyrimidin-4-yl)-2-((((1s,3S)-3-fluorocyclobutyl)amino)methyl)-1-isopropylquinolin-4(1H)-one

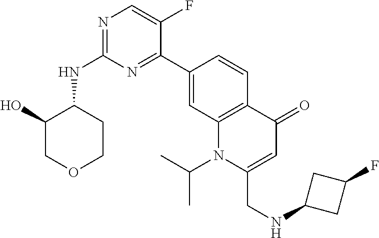 [SMILES: CC(C)n1c(CN[C@H]2C[C@@H](F)C2)cc(=O)c2ccc(-c3nc(N[C@@H]4CCOC[C@H]4O)ncc3F)cc21]

The title compound (12 mg, 43%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (1s,3s)-3-fluorocyclobutan-1-amine. ¹H-NMR (400 MHz, DMSO-d6) δ 8.70 (s, 1H), 8.51 (d, J=3.0 Hz, 1H), 8.30 (d, J=8.3 Hz, 1H), 7.93 (d, J=8.5 Hz, 1H), 7.31 (d, J=7.7 Hz, 1H), 6.21 (s, 1H), 5.35 (s, 1H), 4.97 (s, 1H), 4.84-4.70 (m, 1H), 3.95-3.75 (m, 5H), 3.63-3.43 (m, 2H), 3.32-3.25 (m, 1H), 3.03 (t, J=10.2 Hz, 1H), 2.74 (s, 1H), 2.42-2.12 (m, 4H), 2.10-1.90 (m, 1H), 1.75-1.72 (m, 6H), 1.53-1.51 (m, 1H). LC-MS (M+H)⁺=500.1.

### Example 65: 7-(5-fluoro-2-(((3S,4R)-3-hydroxytetrahydro-2H-pyran-4-yl)amino)pyrimidin-4-yl)-1-isopropyl-2-((((R)-tetrahydrofuran-3-yl)amino)methyl)quinolin-4(1H)-one

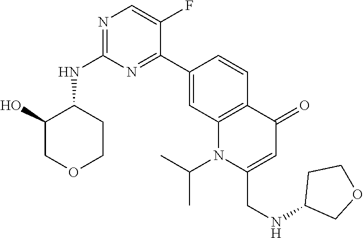 [SMILES: CC(C)n1c(CN[C@@H]2CCOC2)cc(=O)c2ccc(-c3nc(N[C@@H]4CCOC[C@H]4O)ncc3F)cc21]

The title compound (14 mg, 50%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (R)-tetrahydrofuran-3-amine. ¹H-NMR (400 MHz, DMSO-d6) δ 8.70 (s, 1H), 8.51 (d, J=3.0 Hz, 1H), 8.30 (d, J=8.3 Hz, 1H), 7.93 (d, J=8.1 Hz, 1H), 7.31 (d, J=7.5 Hz, 1H), 6.26 (s, 1H), 5.45-5.31 (m, 1H), 4.97 (s, 1H), 3.89-3.83 (m, 5H), 3.82-3.69 (m, 3H), 3.68-3.66 (m, 1H), 3.54 (brs, 1H), 3.48-3.46 (m, 1H), 3.30-3.28 (m, 2H), 3.06-3.01 (m, 1H), 2.02-2.00 (m, 2H), 1.72 (brs, 7H), 1.54-1.51 (m, 1H). LC-MS (M+H)⁺=498.1.

### Example 66: 2-((cyclopentylamino)methyl)-7-(5-fluoro-2-(((3S,4R)-3-hydroxytetrahydro-2H-pyran-4-yl)amino)pyrimidin-4-yl)-1-isopropylquinolin-4(1H)-one

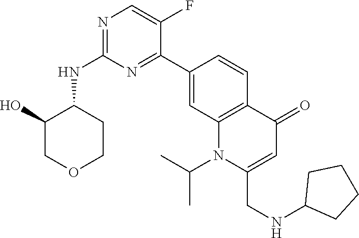 [SMILES: CC(C)n1c(CNC2CCCC2)cc(=O)c2ccc(-c3nc(N[C@@H]4CCOC[C@H]4O)ncc3F)cc21]

The title compound (14 mg, 50%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and cyclopentanamine. ¹H-NMR (400 MHz, DMSO-d6) δ 8.70 (s, 1H), 8.51 (s, 1H), 8.30 (d, J=8.3 Hz, 1H), 7.92 (d, J=8.4 Hz, 1H), 7.31 (d, J=7.7 Hz, 1H), 6.23 (s, 1H), 5.47-5.36 (m, 1H), 4.97 (s, 1H), 3.94-3.71 (m, 5H), 3.64-3.44 (m, 2H), 3.15-2.92 (m, 3H), 2.03-2.00 (m, 1H), 1.72 (brs, 8H), 1.74-1.54 (m, 2H), 1.59-1.39 (m, 3H), 1.49-1.29 (m, 2H). LC-MS (M+H)⁺=496.1.

### Example 67: 7-(5-fluoro-2-(((3S,4R)-3-hydroxytetrahydro-2H-pyran-4-yl)amino)pyrimidin-4-yl)-1-isopropyl-2-((((S)-tetrahydrofuran-3-yl)amino)methyl)quinolin-4(1H)-one

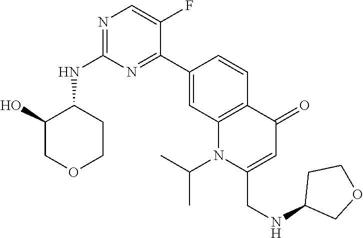 [SMILES: CC(C)n1c(CN[C@H]2CCOC2)cc(=O)c2ccc(-c3nc(N[C@@H]4CCOC[C@H]4O)ncc3F)cc21]

The title compound (13 mg, 46%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-tetrahydrofuran-3-amine. ¹H-NMR (400 MHz, DMSO-d6) δ 8.71 (s, 1H), 8.52 (s, 1H), 8.30 (d, J=8.4 Hz, 1H), 7.93 (d, J=8.2 Hz, 1H), 7.31 (d, J=7.5 Hz, 1H), 6.26 (s, 1H), 5.48-5.28 (m, 1H), 4.97 (s, 1H), 3.85-3.83 (m, 5H), 3.79-3.60 (m, 3H), 3.54 (brs, 1H), 3.48-3.46 (m, 1H), 3.33-3.24 (m, 3H), 3.06-3.01 (m, 1H), 2.05-1.85 (m, 2H), 1.72 (brs, 7H), 1.54-1.51 (m, 1H). LC-MS (M+H)⁺=498.1.

### Example 68: 2-((3-oxa-6-azabicyclo[3.1.1]heptan-6-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

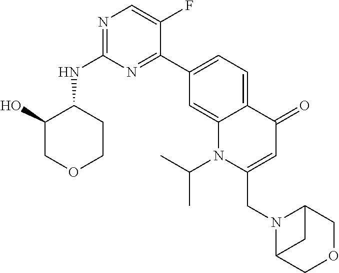 [SMILES: CC(C)n1c(CN2C3COCC2C3)cc(=O)c2ccc(-c3nc(N[C@@H]4CCOC[C@H]4O)ncc3F)cc21]

The title compound (8 mg, 30%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3-oxa-6-azabicyclo[3.1.1]heptane hydrochloride. ¹H-NMR (400 MHz, DMSO-d6) δ: 8.72 (s, 1H), 8.51 (s, 1H), 8.29 (d, J=7.8 Hz, 1H), 7.92 (d, J=8.4 Hz, 1H), 7.31 (d, J=7.0 Hz, 1H), 6.27 (s, 1H), 5.35 (s, 11H), 4.98 (s, 1H), 4.21 (d, J=9.9 Hz, 2H), 4.09-3.75 (m, 51H), 3.68 (d, J=10.3 Hz, 21H), 3.60-3.45 (m, 3H), 3.39 (s, 1H), 3.04 (t, J=10.3 Hz, 1H), 2.69-2.49 (m, 1H), 2.01 (d, J=10.0 Hz, 1H), 1.74 (brs, 7H), 1.60-1.41 (m, 1H). LC-MS (M+H)⁺=510.

### Example 69: 7-(5-fluoro-2-(((3S,4R)-3-hydroxytetrahydro-2H-pyran-4-yl)amino)pyrimidin-4-yl)-2-(((S)-3-fluoropiperidin-1-yl)methyl)-1-isopropylquinolin-4(1H)-one

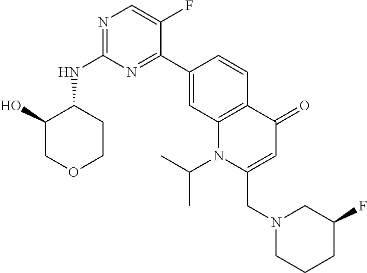 [SMILES: CC(C)n1c(CN2CCC[C@H](F)C2)cc(=O)c2ccc(-c3nc(N[C@@H]4CCOC[C@H]4O)ncc3F)cc21]

The title compound (10.6 mg, 37%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-3-fluoropiperidine hydrochloride. ¹H-NMR (400 MHz, DMSO-d6) δ: 8.74 (s, 1H), 8.52 (s, 1H), 8.31 (d, J=8.2 Hz, 1H), 7.94 (d, J=7.9 Hz, 1H), 7.32 (d, J=7.9 Hz, 1H), 6.18 (s, 1H), 5.39-5.22 (m, 1H), 4.97 (d, J=4.4 Hz, 1H), 4.67 (d, J=49.3 Hz, 1H), 3.97-3.79 (m, 3H), 3.74-3.60 (m, 2H), 3.59-3.46 (m, 1H), 3.31-3.27 (m, 1H), 3.04 (t, J=10.1 Hz, 1H), 2.81-2.65 (m, 1H), 2.62-2.54 (m, 1H), 2.43 (s, 2H), 2.01 (d, J=11.5 Hz, 11H), 1.88-1.61 (m, 8H), 1.66-1.41 (m, 3H). LC-MS (M+H)⁺=514.

### Example 70: 2-((3,3-difluoropiperidin-1-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

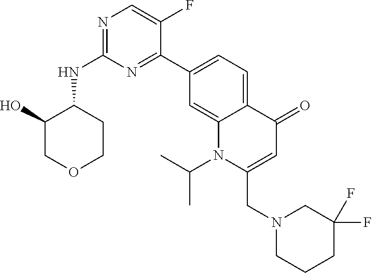 [SMILES: CC(C)n1c(CN2CCCC(F)(F)C2)cc(=O)c2ccc(-c3nc(N[C@@H]4CCOC[C@H]4O)ncc3F)cc21]

The title compound (8.3 mg, 28%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3,3-difluoropiperidine hydrochloride. ¹H-NMR (400 MHz, DMSO-d6) δ: 8.74 (s, 1H), 8.52 (s, 1H), 8.31 (d, J=8.3 Hz, 1H), 7.95 (d, J=8.2 Hz, 1H), 7.32 (d, J=8.2 Hz, 1H), 6.19 (s, 1H), 5.36-5.19 (m, 1H), 4.97 (d, J=4.4 Hz, 1H), 3.98-3.80 (m, 3H), 3.74 (s, 2H), 3.63-3.43 (m, 1H), 3.30 (s, 1H), 3.04 (t, J=10.3 Hz, 1H), 2.77 (t, J=11.4 Hz, 2H), 2.55-2.40 (m, 2H), 2.13-1.80 (m, 3H), 1.80-1.61 (m, 8H), 1.52 (dd, J=21.3, 9.7 Hz, 1H). LC-MS (M+H)⁺=532.

### Example 71: 2-(((3R,5R)-3,5-dimethylmorpholino)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

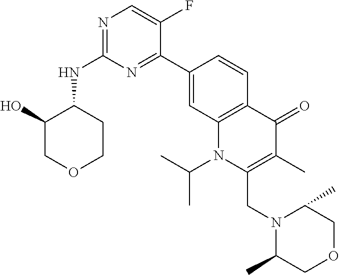 [SMILES: Cc1c(CN2[C@H](C)COC[C@H]2C)n(C(C)C)c2cc(-c3nc(N[C@@H]4CCOC[C@H]4O)ncc3F)ccc2c1=O]

The title compound (1 mg, 5%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (3R,5R)-3,5-dimethylmorpholine hydrochloride. ¹H-NMR (400 MHz, DMSO-d6) δ 8.74 (brs, 1H), 8.51 (d, J=2.7 Hz, 1H), 8.32 (d, J=8.2 Hz, 1H), 7.90 (d, J=8.3 Hz, 1H), 7.31 (d, J=7.6 Hz, 1H), 5.67-5.51 (m, 1H), 4.99 (s, 1H), 4.22-4.15 (m, 1H), 3.96-3.80 (m, 4H), 3.66-3.49 (m, 3H), 3.48-3.28 (m, 3H), 3.04 (t, J=10.4 Hz, 1H), 2.86-2.76 (m, 2H), 2.24 (s, 3H), 2.06-1.96 (m, 1H), 1.82-1.69 (m, 6H), 1.59-1.46 (m, 1H), 1.05-0.98 (m, 6H). LC-MS (M+H)⁺=540.1.

### Example 72: 2-(((3R,5S)-3,5-dimethylmorpholino)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

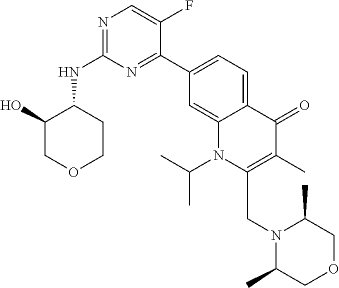 [SMILES: Cc1c(CN2[C@H](C)COC[C@@H]2C)n(C(C)C)c2cc(-c3nc(N[C@@H]4CCOC[C@H]4O)ncc3F)ccc2c1=O]

The title compound (20 mg, 20%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (3R,5S)-3,5-dimethylmorpholine hydrochloride. ¹H-NMR (400 MHz, DMSO-d6) δ 8.73 (brs, 1H), 8.51 (s, 1H), 8.32 (d, J=8.5 Hz, 1H), 7.90 (d, J=8.3 Hz, 1H), 7.30 (d, J=7.8 Hz, 1H), 5.78-5.63 (m, 1H), 4.97 (s, 1H), 4.02 (s, 2H), 3.96-3.79 (m, 3H), 3.64 (d, J=11.1 Hz, 2H), 3.58-3.49 (m, 1H), 3.39-3.30 (m, 11H), 3.21 (t, J=9.9 Hz, 2H), 3.05 (t, J=10.3 Hz, 1H), 2.66-2.57 (m, 2H), 2.25 (s, 3H), 2.09-1.95 (m, 1H), 1.79-1.71 (m, 6H), 1.60-1.45 (m, 1H), 0.98-0.92 (m, 6H). LC-MS (M+H)⁺=540.1.

### Example 73: 2-((7-oxa-4-azaspiro[2.5]octan-4-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

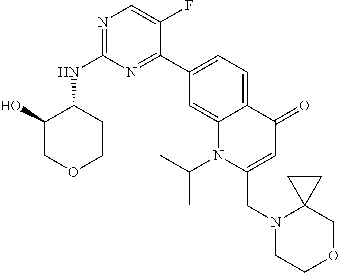 [SMILES: CC(C)n1c(CN2CCOCC23CC3)cc(=O)c2ccc(-c3nc(N[C@@H]4CCOC[C@H]4O)ncc3F)cc21]

The title compound (4 mg, 14%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 7-oxa-4-azaspiro[2.5]octane. ¹H-NMR (400 MHz, DMSO-d6) δ 8.70 (s, 1H), 8.51 (s, 1H), 8.29 (d, J=8.2 Hz, 1H), 7.93 (d, J=8.5 Hz, 1H), 7.31 (d, J=7.7 Hz, 1H), 6.27 (s, 1H), 5.31-5.11 (m, 1H), 4.97 (s, 1H), 3.98 (s, 2H), 3.95-3.75 (m, 3H), 3.67 (s, 2H), 3.59-3.39 (m, 3H), 3.45-3.25 (m, 1H), 3.05 (t, J=10.3 Hz, 1H), 2.79 (s, 2H), 2.09-1.89 (s, 1H), 1.68 (t, J=6.2 Hz, 6H), 1.62-1.42 (m, 1H), 0.71 (s, 2H), 0.51 (s, 2H). LC-MS (M+H)₌524.0.

### Example 74: 7-(5-fluoro-2-(((3S,4R)-3-hydroxytetrahydro-2H-pyran-4-yl)amino)pyrimidin-4-yl)-2-((3-fluoroazetidin-1-yl)methyl)-1-isopropylquinolin-4(1H)-one

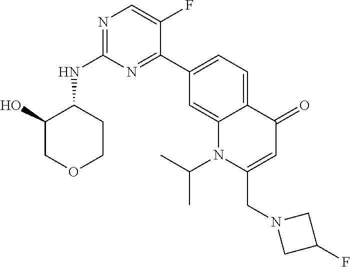 [SMILES: CC(C)n1c(CN2CC(F)C2)cc(=O)c2ccc(-c3nc(N[C@@H]4CCOC[C@H]4O)ncc3F)cc21]

The title compound (8.4 mg, 40%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3-fluoroazetidine hydrochloride. ¹H-NMR (400 MHz, DMSO-d6) δ 8.69 (s, 1H), 8.51 (s, 1H), 8.29 (d, J=8.5 Hz, 1H), 7.93 (d, J=7.8 Hz, 1H), 7.31 (d, J=7.8 Hz, 1H), 6.23 (s, 1H), 5.44-5.12 (m, 2H), 4.97 (s, 1H), 3.97-3.76 (m, 5H), 3.75-3.61 (m, 2H), 3.53 (s, 1H), 3.39-2.19 (m, 2H), 3.03 (t, J=10.0 Hz, 1H), 2.14-1.93 (m, J=13.9 Hz, 1H), 1.71 (s, 7H), 1.58-1.36 (m, 1H). LC-MS (M+H)⁺=486.1.

### Example 75: 2-((tert-butylamino)methyl)-7-(5-fluoro-2-(((3S,4R)-3-hydroxytetrahydro-2H-pyran-4-yl)amino)pyrimidin-4-yl)-1-isopropylquinolin-4(1H)-one

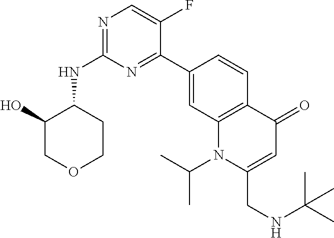 [SMILES: CC(C)n1c(CNC(C)(C)C)cc(=O)c2ccc(-c3nc(N[C@@H]4CCOC[C@H]4O)ncc3F)cc21]

The title compound (17 mg, 55%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and tert-butylamine. ¹H-NMR (400 MHz, DMSO-d6) δ 8.71 (s, 1H), 8.51 (d, J=2.9 Hz, 1H), 8.30 (d, J=8.4 Hz, 1H), 7.92 (d, J=8.3 Hz, 1H), 7.31 (d, J=7.9 Hz, 1H), 6.24 (s, 1H), 5.53-5.30 (m, 1H), 4.97 (s, 1H), 3.99-3.80 (m, 3H), 3.75 (s, 2H), 3.54-3.53 (m, 1H), 3.32 (t, J=10.6 Hz, 2H), 3.04 (t, J=10.4 Hz, 1H), 2.02 (d, J=11.3 Hz, 1H), 1.73 (t, J=6.3 Hz, 6H), 1.54-1.51 (m, 1H), 1.13 (s, 9H). LC-MS (M+H)⁺=484.1.

### Example 76: 2-((cyclobutylamino)methyl)-7-(5-fluoro-2-(((3S,4R)-3-hydroxytetrahydro-2H-pyran-4-yl)amino)pyrimidin-4-yl)-1-isopropylquinolin-4(1H)-one

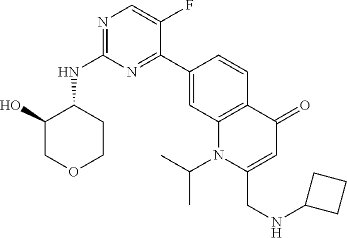 [SMILES: CC(C)n1c(CNC2CCC2)cc(=O)c2ccc(-c3nc(N[C@@H]4CCOC[C@H]4O)ncc3F)cc21]

The title compound (15 mg, 45%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and cyclobutylamine. ¹H-NMR (400 MHz, DMSO-d6) δ 8.70 (s, 1H), 8.51 (s, 1H), 8.30 (d, J=8.3 Hz, 1H), 7.92 (d, J=8.1 Hz, 1H), 7.31 (d, J=7.6 Hz, 1H), 6.20 (s, 1H), 5.51-5.27 (m, 1H), 4.97 (s, 1H), 3.95-3.75 (m, 3H), 3.75 (s, 2H), 3.54 (s, 1H), 3.36-3.30 (m, 2H), 3.25-3.13 (m, 1H), 3.04 (t, J=10.2 Hz, 1H), 2.12-2.00 (m, 3H), 1.82-1.42 (m, 11H). LC-MS (M+H)⁺=482.1.

### Example 77: 2-(((3,3-difluorocyclobutyl)amino)methyl)-7-(5-fluoro-2-(((3S,4R)-3-hydroxytetrahydro-2H-pyran-4-yl)amino)pyrimidin-4-yl)-1-isopropylquinolin-4(1H)-one

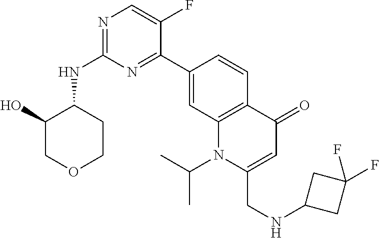 [SMILES: CC(C)n1c(CNC2CC(F)(F)C2)cc(=O)c2ccc(-c3nc(N[C@@H]4CCOC[C@H]4O)ncc3F)cc21]

The title compound (27 mg, 46%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3,3-difluorocyclobutan-1-amine. ¹H-NMR (400 MHz, DMSO-d6) δ 8.71 (s, 1H), 8.51 (s, 1H), 8.30 (d, J=8.4 Hz, 1H), 7.93 (d, J=8.2 Hz, 1H), 7.31 (d, J=8.0 Hz, 1H), 6.25 (s, 1H), 5.41-5.24 (m, 1H), 4.97 (s, 1H), 3.86-3.80 (m, 5H), 3.54 (s, 1H), 3.30-3.21 (m, 2H), 3.04 (t, J=10.4 Hz, 1H), 2.94 (s, 1H), 2.77 (s, 2H), 2.49-2.29 (m, 2H), 2.01 (d, J=12.3 Hz, 1H), 1.72 (s, 6H), 1.53-1.51 (m, 1H). LC-MS (M+H)⁺=518.1.

### Example 78: 7-(5-fluoro-2-(((3S,4R)-3-hydroxytetrahydro-2H-pyran-4-yl)amino)pyrimidin-4-yl)-1-isopropyl-2-(((1-methyl-1H-pyrazol-4-yl)amino)methyl)quinolin-4(1H)-one

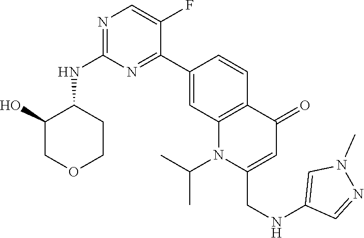 [SMILES: CC(C)n1c(CNc2cnn(C)c2)cc(=O)c2ccc(-c3nc(N[C@@H]4CCOC[C@H]4O)ncc3F)cc21]

The title compound (22 mg, 41%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 1-methyl-1H-pyrazol-4-amine. ¹H-NMR (400 MHz, DMSO-d6) δ 8.70 (s, 1H), 8.52 (s, 1H), 8.29 (d, J=8.1 Hz, 1H), 7.93 (d, J=8.5 Hz, 1H), 7.31 (d, J=7.6 Hz, 1H), 7.15 (s, 1H), 7.03 (s, 1H), 6.31 (s, 1H), 5.21-5.15 (m, 2H), 4.97 (s, 1H), 4.23 (d, J=3.8 Hz, 2H), 3.85-3.83 (m, 3H), 3.68 (s, 3H), 3.53 (s, 1H), 3.33-3.30 (m, 1H), 3.04 (t, J=10.4 Hz, 1H), 2.01 (d, J=11.3 Hz, 1H), 1.73 (s, 6H), 1.54-1.51 (m, 1H). LC-MS (M+H)⁺=508.1.

### Example 79: 2-((1,4-oxazepan-4-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

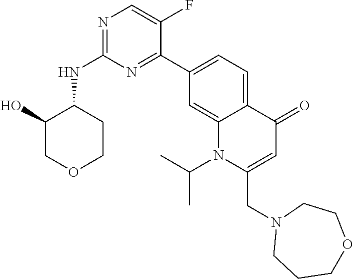 [SMILES: CC(C)n1c(CN2CCCOCC2)cc(=O)c2ccc(-c3nc(N[C@@H]4CCOC[C@H]4O)ncc3F)cc21]

The title compound (15 mg, 52%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 1,4-oxazepane. ¹H-NMR (400 MHz, DMSO-d6) δ 8.74 (s, 1H), 8.52 (s, 1H), 8.30 (d, J=8.2 Hz, 1H), 7.94 (d, J=7.8 Hz, 1H), 7.32 (d, J=8.2 Hz, 1H), 6.18 (s, 1H), 5.50-5.30 (m, 1H), 4.97 (s, 1H), 3.86 (s, 3H), 3.77 (s, 2H), 3.70 (s, 2H), 3.63 (s, 2H), 3.53 (s, 1H), 3.44-2.24 (m, 1H), 3.04 (t, J=10.4 Hz, 1H), 2.71 (s, 4H), 2.10-1.90 (m, 1H), 1.84 (s, 2H), 1.74 (s, 6H), 1.52 (d, J=11.7 Hz, 1H). LC-MS (M+H)⁺=512.0.

### Example 80: 2-((3,3-difluoropyrrolidin-1-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

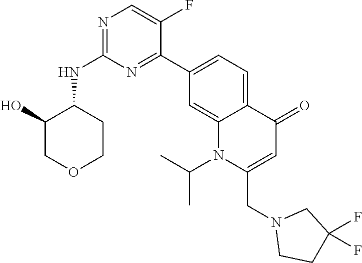 [SMILES: CC(C)n1c(CN2CCC(F)(F)C2)cc(=O)c2ccc(-c3nc(N[C@@H]4CCOC[C@H]4O)ncc3F)cc21]

The title compound (6 mg, 26%) was prepared in a manner similar to at in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3,3-difluoropyrrolidine hydrochloride. ¹H-NMR (400 MHz, DMSO-d6) δ 8.73 (s, 1H), 8.52 (s, 1H), 8.31 (d, J=8.9 Hz, 1H), 7.94 (d, J=8.3 Hz, 1H), 7.32 (d, J=7.2 Hz, 1H), 6.23 (s, 1H), 5.38-5.24 (m, 1H), 5.02-4.92 (m, 1H), 3.99-3.76 (m, 5H), 3.53 (s, 1H), 3.29 (d, J=11.6 Hz, 1H), 3.11-2.93 (m, 3H), 2.85-2.70 (m, 2H), 2.37-2.21 (m, 2H), 2.09-1.96 (m, 1H), 1.71 (s, 6H), 1.57-1.46 (m, 1H). LC-MS (M+H)⁺=518.1.

### Example 81: 2-((3-oxa-8-azabicyclo[3.2.1]octan-8-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

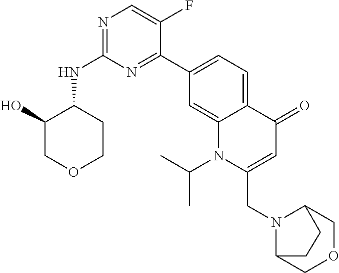 [SMILES: CC(C)n1c(CN2C3CCC2COC3)cc(=O)c2ccc(-c3nc(N[C@@H]4CCOC[C@H]4O)ncc3F)cc21]

The title compound (9 mg, 22%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3-oxa-8-azabicyclo[3.2.1]octane hydrochloride. ¹H-NMR (400 MHz, DMSO-d6) δ 8.74 (brs, 1H), 8.50 (d, J=3.8 Hz, 1H), 8.30 (d, J=8.4 Hz, 1H), 7.93 (d, J=8.3 Hz, 1H), 7.28 (d, J=7.9 Hz, 1H), 6.17 (s, 1H), 5.69-5.53 (m, 1H), 4.95 (d, J=5.1 Hz, 1H), 3.90-3.79 (m, 3H), 3.59-3.32 (m, 8H), 3.15-2.94 (m, 3H), 2.03-1.95 (m, 3H), 1.82-1.72 (m, 8H), 1.56-1.46 (m, 1H). LC-MS (M+H)⁺=524.3.

### Example 82: 7-(5-fluoro-2-(((3S,4R)-3-hydroxytetrahydro-2H-pyran-4-yl)amino)pyrimidin-4-yl)-1-isopropyl-2-(((R)-3-methylmorpholino)methyl)quinolin-4(1H)-one

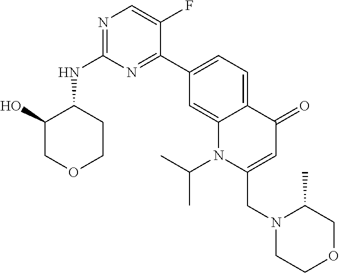 [SMILES: CC(C)n1c(CN2CCOC[C@H]2C)cc(=O)c2ccc(-c3nc(N[C@@H]4CCOC[C@H]4O)ncc3F)cc21]

The title compound (17 mg, 51%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R)-3-methylmorpholine. ¹H-NMR (400 MHz, DMSO-d6) δ 8.73 (brs, 1H), 8.52 (d, J=3.8 Hz, 1H), 8.30 (d, J=8.4 Hz, 1H), 7.94 (d, J=8.4 Hz, 1H), 7.31 (d, J=7.9 Hz, 1H), 6.25-6.15 (m, 1H), 5.53-5.42 (m, 1H), 4.97 (d, J=5.0 Hz, 1H), 4.32-4.22 (m, 1H), 3.90-3.80 (m, 3H), 3.72-3.60 (m, 2H), 3.60-3.44 (m, 2H), 3.33-3.17 (m, 3H), 3.11-2.98 (m, 1H), 2.80-2.64 (m, 1H), 2.63-2.53 (m, 1H). 2.34-2.19 (m, 1H), 2.07-1.96 (m, 1H), 1.92-1.67 (m, 6H), 1.61-1.47 (m, 1H), 1.12-1.00 (m, 3H). LC-MS (M+H)⁺=512.3.

### Example 83: 2-((1,1-dioxidothiomorpholino)methyl)-7-(5-fluoro-2-(((3S,4R)-3-hydroxytetrahydro-2H-pyran-4-yl)amino)pyrimidin-4-yl)-1-isopropylquinolin-4(1H)-one

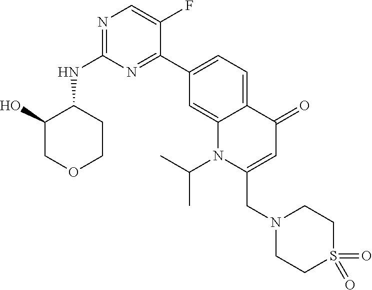 [SMILES: CC(C)n1c(CN2CCS(=O)(=O)CC2)cc(=O)c2ccc(-c3nc(N[C@@H]4CCOC[C@H]4O)ncc3F)cc21]

The title compound (14 mg, 41%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and thiomorpholine 1,1-dioxide hydrochloride. ¹H-NMR (400 MHz, DMSO-d6) δ 8.76 (brs, 1H), 8.52 (d, J=3.8 Hz, 1H), 8.31 (d, J=8.4 Hz, 1H), 7.94 (d, J=8.4 Hz, 1H), 7.32 (d, J=8.0 Hz, 1H), 6.24 (s, 1H), 5.34-5.22 (m, 1H), 4.99 (s, 1H), 4.01-3.76 (m, 5H), 3.57-3.49 (m, 1H), 3.38-3.29 (m, 1H), 3.18-3.11 (m, 4H), 3.09-2.97 (m, 5H), 2.05-1.98 (m, 1H), 1.78-1.70 (m, 6H), 1.60-1.46 (m, 1H). LC-MS (M+H)⁺=546.2.

### Example 84 and Example 85: 7-(5-fluoro-2-(((3S,4R)-3-hydroxytetrahydro-2H-pyran-4-yl)amino)pyrimidin-4-yl)-2-(((3R,4R)-4-fluoro-3-hydroxypiperidin-1-yl)methyl)-1-isopropylquinolin-4(1H)-one & 7-(5-fluoro-2-(((3S,4R)-3-hydroxytetrahydro-2H-pyran-4-yl)amino)pyrimidin-4-yl)-2-(((3S,4S)-4-fluoro-3-hydroxypiperidin-1-yl)methyl)-1-isopropylquinolin-4(1H)-one

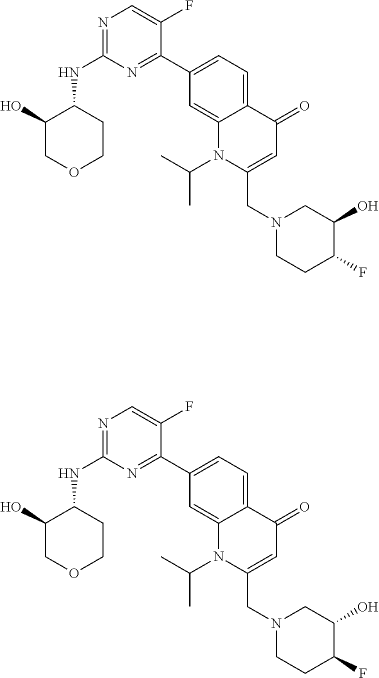 [SMILES: CC(C)n1c(CN2CC[C@@H](F)[C@H](O)C2)cc(=O)c2ccc(-c3nc(N[C@@H]4CCOC[C@H]4O)ncc3F)cc21 ; CC(C)n1c(CN2CC[C@H](F)[C@@H](O)C2)cc(=O)c2ccc(-c3nc(N[C@@H]4CCOC[C@H]4O)ncc3F)cc21]

The title compounds Example 84 and Example 85 were prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and trans-4-fluoro-3-piperidinol. The 2 isomeric products were separated on chiral-HPLC to give 7-(5-fluoro-2-(((3S,4R)-3-hydroxytetrahydro-2H-pyran-4-yl)amino)pyrimidin-4-yl)-2-(((3R,4R)-4-fluoro-3-hydroxypiperidin-1-yl)methyl)-1-isopropylquinolin-4(1H)-one & 7-(5-fluoro-2-(((3S,4R)-3-hydroxytetrahydro-2H-pyran-4-yl)amino)pyrimidin-4-yl)-2-(((3S,4S)-4-fluoro-3-hydroxypiperidin-1-yl)methyl)-1-isopropylquinolin-4(1H)-one. Analytical chiral HPLC condition: CHIRALPAK ID-3, 0.46×5 cm, 3.0 um. Mobile phase: (hexane: dichloromethane=3:1) ((0.5% 2M NH₃ in methanol)): ethanol, 20 mL/min in 13 min.

Example 84: (5 mg, 10%) ¹H-NMR (300 MHz, DMSO-d6) δ 8.74 (brs, 1H), 8.52 (d, J=3.8 Hz, 1H), 8.31 (d, J=8.4 Hz, 1H), 7.94 (d, J=8.4 Hz, 1H), 7.31 (d. J=7.9 Hz, 1H), 6.18 (s, 1H), 5.37-5.26 (m, 1H), 5.21 (d, J=4.7 Hz, 1H), 4.97 (d, J=5.1 Hz, 1H), 4.39-4.15 (m, 1H), 3.91-3.80 (m, 3H), 3.74-3.48 (m, 4H), 3.38 (s, 1H), 3.11-2.98 (m, 1H), 2.93-2.76 (m, 2H), 2.23-2.10 (m, 1H), 2.07-1.94 (m, 3H), 1.78-1.43 (m, 8H). LC-MS (M+H)⁺=530.3. Chiral HPLC: RT=2.851 min.

Example 85: (2 mg, 3%) ¹H-NMR (300 MHz, DMSO-d6) δ 8.74 (brs, 1H), 8.55-8.49 (m, 1H), 8.36-8.27 (m, 1H), 7.98-7.92 (m, 1H), 7.34-7.28 (m, 1H), 6.18 (s, 1H), 5.35-5.16 (m, 2H), 5.00-4.94 (m, 1H), 4.39-4.11 (m, 1H), 3.89-3.83 (m, 3H), 3.76-3.40 (m, 4H), 3.33-3.28 (m, 1H), 3.08-3.02 (m, 1H), 2.88-2.82 (m, 2H), 2.20-2.14 (m, 1H), 2.08-1.97 (m, 3H), 1.79-1.48 (m, 8H). LC-MS (M+H)⁺=530.3. Chiral HPLC: RT=3.729 min.

### Example 86: 7-(5-fluoro-2-(((3S,4R)-3-hydroxytetrahydro-2H-pyran-4-yl)amino)pyrimidin-4-yl)-2-(((S)-3-hydroxypyrrolidin-1-yl)methyl)-1-isopropylquinolin-4(1H)-one

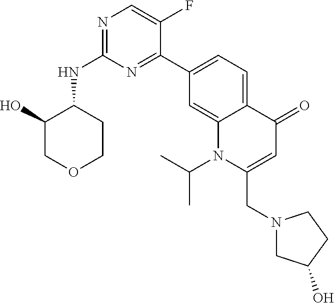 [SMILES: CC(C)n1c(CN2CC[C@H](O)C2)cc(=O)c2ccc(-c3nc(N[C@@H]4CCOC[C@H]4O)ncc3F)cc21]

The title compound (7 mg, 20%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (S)-pyrrolidin-3-ol. ¹H-NMR (400 MHz, DMSO-d6) δ 8.73 (s, 1H), 8.52 (d, J=3.7 Hz, 1H), 8.31 (d, J=8.4 Hz, 1H), 7.97-7.90 (m, 1H), 7.32 (d, J=8.0 Hz, 1H), 6.21 (s, 1H), 5.44-5.31 (m, 1H), 4.98 (d, J=5.1 Hz, 1H), 4.73 (d, J=4.0 Hz, 1H), 4.26-4.19 (m, 1H), 3.97-3.74 (m, 4H), 3.73-3.65 (m, 1H), 3.60-3.48 (m, 1H), 3.38-3.27 (m, 1H), 3.09-2.99 (m, 1H), 2.80-2.66 (m, 2H), 2.51-2.45 (m, 1H), 2.45-2.37 (m, 1H), 2.07-1.94 (m, 2H), 1.76-1.67 (m, 6H), 1.59-1.49 (m, 2H). LC-MS (M+H)⁺=498.3.

### Example 87: 7-(5-fluoro-2-(((3S,4R)-3-hydroxytetrahydro-2H-pyran-4-yl)amino)pyrimidin-4-yl)-2-(((R)-3-hydroxypyrrolidin-1-yl)methyl)-1-isopropylquinolin-4(1H)-one

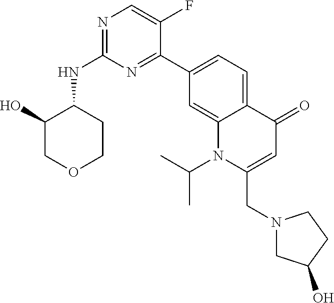 [SMILES: CC(C)n1c(CN2CC[C@@H](O)C2)cc(=O)c2ccc(-c3nc(N[C@@H]4CCOC[C@H]4O)ncc3F)cc21]

The title compound (16 mg, 43%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (R)-pyrrolidin-3-ol. ¹H-NMR (400 MHz, DMSO-d6) δ 8.73 (brs, 1H), 8.52 (d, J=3.8 Hz, 1H), 8.31 (d, J=8.4 Hz, 1H), 7.97-7.90 (m, 1H). 7.31 (d. J=8.0 Hz, 1H), 6.21 (s, 1H), 5.44-5.33 (m, 1H), 4.97 (d, J=5.2 Hz, 1H), 4.73 (d, J=4.0 Hz, 1H), 4.25-4.20 (m, 1H), 3.89-3.75 (m, 4H), 3.73-3.65 (m, 1H), 3.59-3.48 (m, 1H), 3.31-3.28 (m, 1H), 3.09-2.99 (m, 1H), 2.80-2.63 (m, 2H), 2.51-2.37 (m, 2H), 2.10-1.94 (m, 2H), 1.74-1.68 (m, 6H), 1.63-1.46 (m, 2H). LC-MS (M+H)⁺=498.3.

### Example 88: (R)-1-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)piperidine-3-carbonitrile

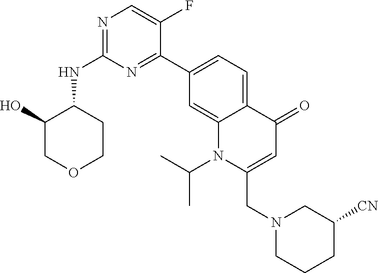 [SMILES: CC(C)n1c(CN2CCC[C@@H](C#N)C2)cc(=O)c2ccc(-c3nc(N[C@@H]4CCOC[C@H]4O)ncc3F)cc21]

The title compound (18 mg, 45%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R)-piperidine-3-carbonitrile hydrochloride. ¹H-NMR (400 MHz, DMSO-d6) δ 8.75 (brs, 1H), 8.52 (d, J=3.8 Hz, 1H), 8.31 (d, J=8.4 Hz, 1H), 7.98-7.91 (m, 1H), 7.32 (d, J=8.0 Hz, 1H), 6.18 (s, 1H), 5.43-5.32 (m, 1H), 4.98 (d, J=5.2 Hz, 1H), 4.00-3.78 (m, 3H), 3.72 (d, J=13.8 Hz, 1H), 3.64-3.47 (m, 2H), 3.35-3.27 (m, 1H), 3.11-2.99 (m, 2H), 2.82-2.77 (m, 1H), 2.61-2.57 (m, 2H), 2.38-2.23 (m, 1H), 2.05-1.97 (m, 1H), 1.82-1.66 (m, 9H), 1.61-1.45 (m, 2H). LC-MS (M+H)⁺=521.3.

### Example 89: (S)-1-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)piperidine-3-carbonitrile

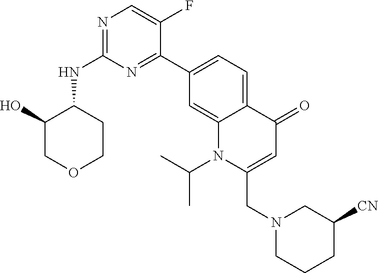 [SMILES: CC(C)n1c(CN2CCC[C@H](C#N)C2)cc(=O)c2ccc(-c3nc(N[C@@H]4CCOC[C@H]4O)ncc3F)cc21]

The title compound (19 mg, 48%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S)-piperidine-3-carbonitrile hydrochloride. ¹H-NMR (400 MHz, DMSO-d6) δ 8.75 (brs, 1H), 8.52 (d, J=3.8 Hz, 1H), 8.31 (d, J=8.5 Hz, 1H), 7.98-7.91 (m, 1H), 7.32 (d, J=8.0 Hz, 1H), 6.18 (s, 1H), 5.43-5.31 (m, 1H), 4.98 (d, J=5.2 Hz, 1H), 4.00-3.79 (m, 3H), 3.76-3.68 (m, 1H), 3.64-3.48 (m, 2H), 3.35-3.30 (m, 1H), 3.10-2.95 (m, 2H), 2.82-2.77 (m, 1H), 2.61-2.57 (m, 2H), 2.37-2.26 (m, 1H), 2.05-1.97 (m, 1H), 1.79-1.65 (m, 9H), 1.60-1.45 (m, 2H). LC-MS (M+H)₌521.3.

### Example 90: 7-(5-fluoro-2-(((3S,4R)-3-hydroxytetrahydro-2H-pyran-4-yl)amino)pyrimidin-4-yl)-2-(((1R,3R,5S)-3-hydroxy-9-azabicyclo[3.3.1]nonan-9-yl)methyl)-1-isopropylquinolin-4(1H)-one

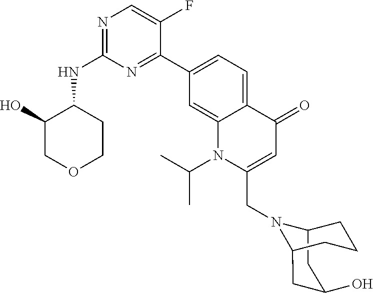 [SMILES: CC(C)n1c(CN2C3CCCC2CC(O)C3)cc(=O)c2ccc(-c3nc(N[C@@H]4CCOC[C@H]4O)ncc3F)cc21]

The title compound (17 mg, 22%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (1R,3r,5S)-9-azabicyclo[3.3.1]nonan-3-ol hydrochloride. ¹H-NMR (300 MHz, DMSO-d6) δ 8.71 (s, 11H), 8.50 (d, J=3.8 Hz, 1H), 8.29 (d, J=8.4 Hz, 11H), 7.97-7.87 (m, 1H), 7.36-7.25 (m, 1H), 6.19 (s, 1H), 5.44-5.28 (m, 1H), 4.99-4.93 (m, 1H), 4.53-4.47 (m, 1H), 4.01-3.78 (m, 4H), 3.56-3.50 (m, 1H), 3.40-3.26 (m, 3H), 3.10-2.93 (m, 3H), 2.40-2.34 (m, 1H), 2.30-2.14 (m, 2H), 2.06-1.95 (m, 1H), 1.94-1.81 (m, 2H), 1.79-1.66 (m, 6H), 1.61-1.36 (m, 2H), 1.33-1.21 (m, 2H), 1.19-1.09 (m, 2H). LC-MS (M+H)⁺=552.4.

### Example 91: 2-((1-oxa-7-azaspiro[4.4]nonan-7-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

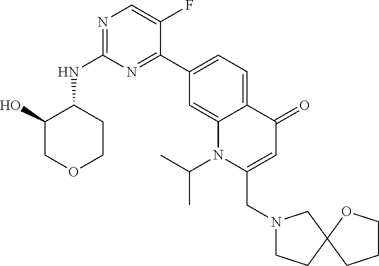 [SMILES: CC(C)n1c(CN2CCC3(CCCO3)C2)cc(=O)c2ccc(-c3nc(N[C@@H]4CCOC[C@H]4O)ncc3F)cc21]

The title compound (2.2 mg, 9%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 1-oxa-7-azaspiro[4.4]nonane. ¹H-NMR (400 MHz, DMSO-d6) δ 8.72 (s, 1H), 8.52 (s, 1H), 8.30 (d, J=7.9 Hz, 1H), 7.93 (d, J=7.8 Hz, 1H), 7.31 (d, J=7.6 Hz, 1H), 6.28-6.15 (m, 1H), 5.49-5.29 (m, 1H), 5.04-4.89 (m, 1H), 3.96-3.74 (m, 4H), 3.73-3.60 (m, J=13.1 Hz, 3H), 3.53 (s, 1H), 3.44-3.24 (m, 1H), 3.04 (t, J=10.4 Hz, 1H), 2.74-2.61 (m, J=7.8 Hz, 2H), 2.58 (s, 2H), 2.08-1.96 (m, 1H), 1.93-1.77 (m, 6H), 1.75-1.66 (m, 6H), 1.62-1.44 (m, 1H). LC-MS (M+H)⁺=538.1.

### Example 92: 7-(5-fluoro-2-(((3S,4R)-3-hydroxytetrahydro-2H-pyran-4-yl)amino)pyrimidin-4-yl)-2-(((R)-3-fluoropyrrolidin-1-yl)methyl)-1-isopropyl-3-methylquinolin-4(1H)-one

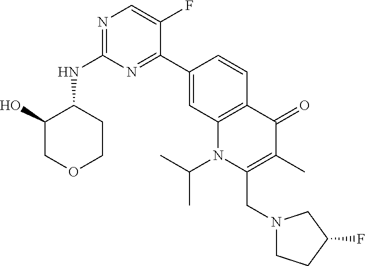 [SMILES: Cc1c(CN2CC[C@@H](F)C2)n(C(C)C)c2cc(-c3nc(N[C@@H]4CCOC[C@H]4O)ncc3F)ccc2c1=O]

The title compound (30 mg, 58%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (R)-3-fluoropyrrolidine. ¹H-NMR (400 MHz, DMSO-d6) δ 8.73 (brs, 1H), 8.51 (s, 1H), 8.34 (d, J=8.2 Hz, 1H), 7.90 (d, J=8.8 Hz, 1H), 7.30 (d, J=8.0 Hz, 1H), 5.56-5.43 (m, 1H), 5.3-5.11 (m, 1H), 4.97 (d, J=5.1 Hz, 1H), 3.97 (s, 2H), 3.93-3.79 (m, 3H), 3.58-3.48 (m, 1H), 3.37-3.28 (s, 1H), 3.03 (t, J=10.5 Hz, 1H), 2.97-2.86 (m, 2H), 2.83-2.65 (m, 1H), 2.51-2.41 (m, 1H), 2.22-2.10 (m, 4H), 2.06-1.79 (m, 2H), 1.77-1.67 (m, 6H), 1.58-1.48 (m, 1H). LC-MS (M+H)⁺=514.1.

### Example 93: 2-(((3S,5S)-3,5-dimethylmorpholino)methyl)-7-(5-fluoro-2-(((3S,4R)-3-hydroxytetrahydro-2H-pyran-4-yl)amino)pyrimidin-4-yl)-1-isopropylquinolin-4(1H)-one

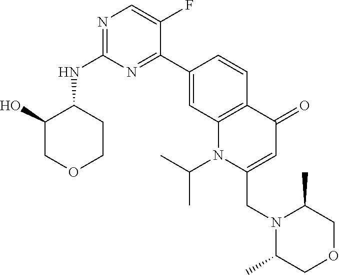 [SMILES: CC(C)n1c(CN2[C@@H](C)COC[C@@H]2C)cc(=O)c2ccc(-c3nc(N[C@@H]4CCOC[C@H]4O)ncc3F)cc21]

The title compound (20 mg, 90%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3S,5S)-3,5-dimethylmorpholine hydrogen chloride salt. ¹H-NMR (400 MHz, DMSO-d6) δ 8.74 (brs, 1H), 8.52 (d, J=3.7 Hz, 1H), 8.30 (d, J=8.4 Hz, 1H), 7.94 (d. J=8.2 Hz, 1H), 7.32 (d, J=8.2 Hz, 1H), 6.32 (s, 1H), 5.69-5.56 (m, 1H), 4.97 (s, 1H), 4.29-4.22 (m, 1H), 3.97-3.78 (m, 3H), 3.65-3.58 (m, 2H), 3.58-3.49 (m, 1H), 3.47-3.40 (m, 1H), 3.38-3.26 (m, 3H), 3.04 (t, J=10.2 Hz, 1H), 2.81-2.71 (m, 2H), 2.06-1.96 (m, 1H), 1.80-1.68 (m, 6H), 1.59-1.46 (m, 1H), 1.08-1.02 (m, 6H). LC-MS (M+H)⁺=526.1.

### Example 94: 7-(5-fluoro-2-(((3S,4R)-3-hydroxytetrahydro-2H-pyran-4-yl)amino)pyrimidin-4-yl)-1-isopropyl-2-((phenylamino)methyl)quinolin-4(1H)-one

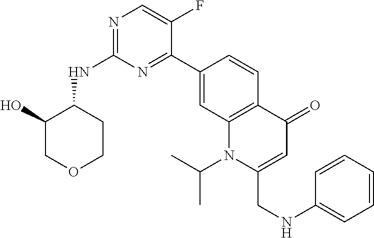 [SMILES: CC(C)n1c(CNc2ccccc2)cc(=O)c2ccc(-c3nc(N[C@@H]4CCOC[C@H]4O)ncc3F)cc21]

The title compound (14 mg, 50%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and aniline. ¹H-NMR (400 MHz, DMSO-d6) δ 8.70 (s, 1H), 8.52 (s, 1H), 8.29 (d, J=8.6 Hz, 1H), 7.93 (d, J=8.1 Hz, 1H), 7.31 (d, J=7.2 Hz, 1H), 7.11 (t, J=7.3 Hz, 2H), 6.65 (d, J=8.0 Hz, 2H), 6.59 (s, 1H), 6.38 (s, 1H), 6.30 (s, 1H), 5.22-5.02 (m, 1H), 4.97 (s, 1H), 4.46 (s, 2H), 3.95-3.75 (m, 3H), 3.53 (s, 1H), 3.31-3.29 (m, 1H), 3.03 (t, J=10.6 Hz, 1H), 2.00 (s, 1H), 1.75 (s, 6H), 1.54 (s, 1H). LC-MS (M+H)⁺=504.1.

### Example 95: 2-(((3-chlorophenyl)amino)methyl)-7-(5-fluoro-2-(((3S,4R)-3-hydroxytetrahydro-2H-pyran-4-yl)amino)pyrimidin-4-yl)-1-isopropylquinolin-4(1H)-one

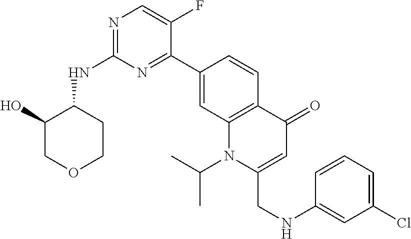 [SMILES: CC(C)n1c(CNc2cccc(Cl)c2)cc(=O)c2ccc(-c3nc(N[C@@H]4CCOC[C@H]4O)ncc3F)cc21]

The title compound (14 mg, 50%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 3-chloroaniline. ¹H-NMR (400 MHz, DMSO-d6) δ 8.71 (s, 11H), 8.52 (s, 1H), 8.30 (d, J=8.1 Hz, 1H), 7.94 (d. J=8.8 Hz, 1H), 7.32 (d, J=7.3 Hz, 1H), 7.12 (t, J=7.6 Hz, 1H), 6.70 (d, J=9.0 Hz, 2H), 6.62 (d, J=7.8 Hz, 2H), 6.28 (s, 1H), 5.16-4.96 (m, 1H), 4.97 (s, 1H), 4.51 (s, 2H), 3.96-3.76 (m, 3H), 3.54 (s, 1H), 3.31-3.27 (m, 1H), 3.04 (t, J=10.3 Hz, 1H), 2.00 (s, 1H), 1.74 (s, 6H), 1.54-1.51 (m, 1H). LC-MS (M+H)⁺=538.1.

### Example 96: 7-(5-fluoro-2-(((3S,4R)-3-hydroxytetrahydro-2H-pyran-4-yl)amino)pyrimidin-4-yl)-2-(((4-fluorophenyl)amino)methyl)-1-isopropylquinolin-4(1H)-one

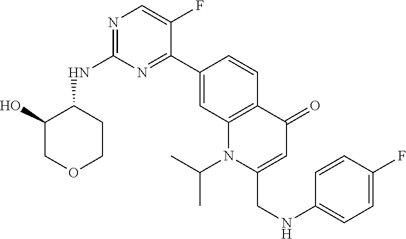 [SMILES: CC(C)n1c(CNc2ccc(F)cc2)cc(=O)c2ccc(-c3nc(N[C@@H]4CCOC[C@H]4O)ncc3F)cc21]

The title compound (13 mg, 45%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4-fluoroaniline. ¹H-NMR (400 MHz, DMSO-d6) δ 8.70 (brs, 1H), 8.52 (s, 1H), 8.29 (d, J=8.2 Hz, 1H), 7.93 (d, J=8.2 Hz, 1H), 7.31 (d, J=7.5 Hz, 1H), 6.96 (t, J=7.9 Hz, 2H), 6.65 (s, 2H), 6.33 (s, 1H), 6.29 (s, 1H), 5.20-5.00 (m, 1H), 4.97 (s, 1H), 4.45 (s, 2H), 3.95-3.75 (m, 3H), 3.54 (s, 1H), 3.29 (s, 1H), 3.03 (t, J=10.0 Hz, 1H), 2.09-1.89 (m, 1H), 1.74 (s, 6H), 1.54-1.51 (m, 1H). LC-MS (M+H)⁺=522.1.

### Example 97: 2-((4,4-dimethylpiperidin-1-yl)methyl)-7-(5-fluoro-2-(((3S,4R)-3-hydroxytetrahydro-2H-pyran-4-yl)amino)pyrimidin-4-yl)-1-isopropylquinolin-4(1H)-one

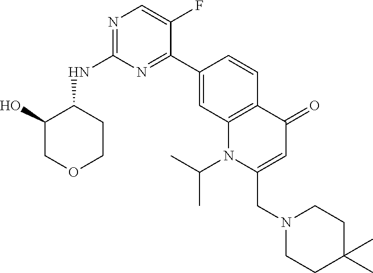 [SMILES: CC(C)n1c(CN2CCC(C)(C)CC2)cc(=O)c2ccc(-c3nc(N[C@@H]4CCOC[C@H]4O)ncc3F)cc21]

The title compound (13 mg, 45%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4,4-dimethylpiperidine. ¹H-NMR (400 MHz, DMSO-d6) δ 8.73 (s, 1H), 8.52 (s, 1H), 8.30 (d, J=8.2 Hz, 1H), 7.94 (d, J=7.8 Hz, 1H), 7.31 (d, J=7.8 Hz, 1H), 6.17 (s, 1H), 5.36 (s, 1H), 4.97 (s, 1H), 3.96-3.76 (m, 3H), 3.63 (s, 2H), 3.54 (s, 1H), 3.34-3.28 (m, 1H), 3.04 (t, J=10.2 Hz, 1H), 2.43 (s, 4H), 2.00 (s, 1H), 1.71 (d, J=6.5 Hz, 6H), 1.54-1.51 (m, 1H), 1.34 (s, 4H), 0.91 (s, 6H). LC-MS (M+H)⁺=524.1.

### Example 98: 7-(5-fluoro-2-(((3S,4R)-3-hydroxytetrahydro-2H-pyran-4-yl)amino)pyrimidin-4-yl)-2-(((R)-3-fluoropiperidin-1-yl)methyl)-1-isopropylquinolin-4(1H)-one

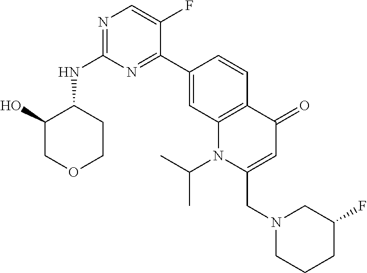 [SMILES: CC(C)n1c(CN2CCC[C@@H](F)C2)cc(=O)c2ccc(-c3nc(N[C@@H]4CCOC[C@H]4O)ncc3F)cc21]

The title compound (13 mg, 45%) was prepared in a manner similar to Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (R)-3-fluoropiperidine hydrochloride. ¹H-NMR (400 MHz, DMSO-d6) δ: 8.74 (s, 1H), 8.51 (s, 1H), 8.31 (d, J=8.4 Hz, 1H), 7.94 (d, J=8.0 Hz, 1H), 7.32 (d, J=7.5 Hz, 1H), 6.18 (s, 1H), 5.39-5.25 (m, 1H), 4.96 (s, 1H), 4.67 (d, J=48.5 Hz, 1H), 4.09-3.73 (m, 3H), 3.73-3.60 (m, 2H), 3.59-3.49 (m, 1H), 3.31 (s, 1H), 3.03 (t, J=10.1 Hz, 1H), 2.81-2.64 (m, 1H), 2.62-2.54 (m, 1H), 2.42 (s, 2H), 2.01 (d, J=10.7 Hz, 1H), 1.88-1.67 (m, 8H), 1.67-1.36 (m, 3H). LC-MS (M+H)⁺=514.

### Example 99: 7-(5-fluoro-2-(((3S,4R)-3-hydroxytetrahydro-2H-pyran-4-yl)amino)pyrimidin-4-yl)-2-((4-fluoropiperidin-1-yl)methyl)-1-isopropylquinolin-4(1H)-one

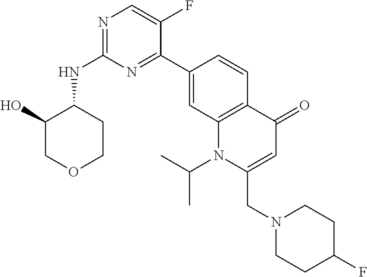 [SMILES: CC(C)n1c(CN2CCC(F)CC2)cc(=O)c2ccc(-c3nc(N[C@@H]4CCOC[C@H]4O)ncc3F)cc21]

The title compound (12 mg, 42%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4-fluoropiperidine. ¹H-NMR (400 MHz, DMSO-d6) δ: 8.74 (s, 1H), 8.52 (s, 1H), 8.30 (d, J=8.3 Hz, 1H), 7.94 (d, J=8.2 Hz, 1H), 7.32 (d, J=7.9 Hz, 1H), 6.17 (s, 1H), 5.41-5.29 (m, 1H), 4.97 (s, 1H), 4.72 (d, J=48.9 Hz, 1H), 3.98-3.77 (m, 3H), 3.64 (s, 2H), 3.60-3.49 (m, 1H), 3.35 (s, 1H), 3.04 (t, J=10.2 Hz, 1H), 2.70-2.55 (m, 2H), 2.47-2.35 (m, 2H), 2.01 (d, J=12.4 Hz, 1H), 1.95-1.79 (m, 2H), 1.73 (s, 8H), 1.60-1.39 (m, 1H). LC-MS (M+H)⁺=514.

### Example 100: 7-(5-fluoro-2-(((3S,4R)-3-hydroxytetrahydro-2H-pyran-4-yl)amino)pyrimidin-4-yl)-1-isopropyl-2-(pyrrolidin-1-ylmethyl)quinolin-4(1H)-one

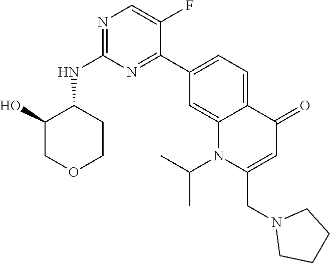 [SMILES: CC(C)n1c(CN2CCCC2)cc(=O)c2ccc(-c3nc(N[C@@H]4CCOC[C@H]4O)ncc3F)cc21]

The title compound (6.2 mg, 26%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and pyrrolidine. ¹H-NMR (400 MHz, DMSO-d6) δ 8.72 (s, 1H), 8.51 (s, 1H), 8.30 (d, J=7.8 Hz, 1H), 7.93 (d, J=8.9 Hz, 1H), 7.31 (d, J=7.4 Hz, 1H), 6.20 (s, 1H), 5.55-5.30 (m, 1H), 4.97 (s, 1H), 4.05-3.80 (m, 3H), 3.75 (s, 2H), 3.53 (s, 1H). 3.34-3.30 (m, 5H), 3.04 (t, J=10.4 Hz, 1H), 2.08-1.95 (m, 1H), 1.80-1.65 (m, 10H), 1.60-1.44 (m, 1H). LC-MS (M+H)⁺=482.1.

### Example 101: 7-(5-fluoro-2-(((3S,4R)-3-hydroxytetrahydro-2H-pyran-4-yl)amino)pyrimidin-4-yl)-1-isopropyl-2-(((4-methoxyphenyl)amino)methyl)quinolin-4(1H)-one

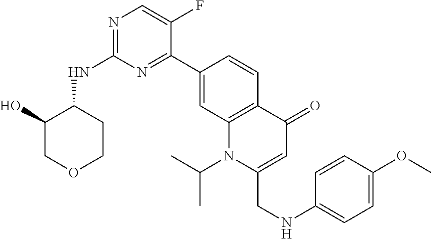 [SMILES: COc1ccc(NCc2cc(=O)c3ccc(-c4nc(N[C@@H]5CCOC[C@H]5O)ncc4F)cc3n2C(C)C)cc1]

The title compound (12 mg, 40%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and 4-methoxyaniline. ¹H-NMR (400 MHz, DMSO-d6) δ 8.70 (s, 1H), 8.52 (s, 1H), 8.29 (d, J=8.7 Hz, 1H), 7.93 (d, J=7.8 Hz, 1H), 7.31 (d, J=7.9 Hz, 1H), 6.74 (d, J=8.1 Hz, 2H), 6.61 (d, J=7.3 Hz, 2H), 6.30 (s, 1H), 6.00 (s, 1H), 5.13 (s, 1H), 4.97 (s, 1H), 4.41 (s, 2H), 3.92 -3.72 (m, 3H), 3.63 (s, 3H), 3.53 (s, 1H), 3.35-3.15 (m, 11H), 3.06-3.03 (m, 1H), 2.00 (brs, 1H), 1.74 (brs, 6H), 1.54-1.51 (m, 1H). LC-MS (M+H)⁺=534.1.

### Example 102: 2-((8-oxa-3-azabicyclo[3.2.1]octan-3-yl)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

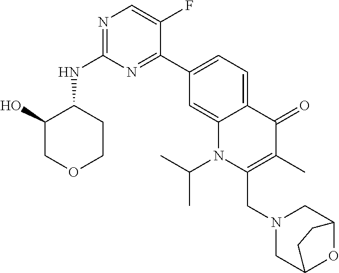 [SMILES: Cc1c(CN2CC3CCC(C2)O3)n(C(C)C)c2cc(-c3nc(N[C@@H]4CCOC[C@H]4O)ncc3F)ccc2c1=O]

The title compound (17 mg, 40%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 8-oxa-3-azabicyclo[3.2.1]octane. ¹H-NMR (400 MHz, DMSO-d6) δ 8.73 (brs, 1H), 8.51 (d, J=3.8 Hz, 1H), 8.34 (d, J=8.4 Hz, 1H), 7.96-7.86 (m, 1H), 7.29 (d, J=8.0 Hz, 1H), 5.65-5.49 (m, 1H), 4.96 (d, J=5.2 Hz, 1H), 4.28-4.22 (m, 2H), 4.01-3.79 (m, 3H), 3.70 (s, 2H), 3.62-3.47 (m, 1H), 3.42-3.32 (m, 1H), 3.12-2.99 (m, 1H), 2.70-2.61 (m, 2H), 2.51-2.45 (m, 2H), 2.17 (s, 3H), 2.08-1.98 (m, 1H), 1.91-1.68 (m, 10H), 1.63-1.43 (m, 11H). LC-MS (M+H)⁺=538.2.

### Example 103: 7-(5-fluoro-2-(((3S,4R)-3-hydroxytetrahydro-2H-pyran-4-yl)amino)pyrimidin-4-yl)-2-((4-hydroxy-4-methylpiperidin-1-yl)methyl)-1-isopropyl-3-methylquinolin-4(1H)-one

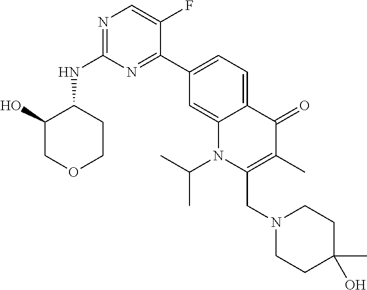 [SMILES: Cc1c(CN2CCC(C)(O)CC2)n(C(C)C)c2cc(-c3nc(N[C@@H]4CCOC[C@H]4O)ncc3F)ccc2c1=O]

The title compound (13 mg, 30%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 4-methylpiperidin-4-ol. ¹H-NMR (400 MHz, DMSO-d6) S 8.72 (brs, 1H), 8.51 (d, J=3.8 Hz, 11H), 8.34 (d, J=8.5 Hz, 11H), 7.95-7.86 (m, 1H), 7.28 (d, J=7.9 Hz, 1H), 5.58-5.43 (m, 1H), 4.96 (d, J=5.2 Hz, 1H), 4.18 (s, 1H), 4.00-3.73 (m, 5H), 3.62-3.47 (m, 1H), 3.42-3.27 (m, 1H), 3.11-2.98 (m, 1H), 2.65-2.52 (m, 4H), 2.18 (s, 3H), 2.11-1.98 (m, 1H), 1.79-1.68 (m, 6H), 1.63-1.38 (m, 5H), 1.11 (s, 3H). LC-MS (M+H)⁺=540.2.

### Example 104 and Example 105: (R)-7-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)-2,7-diazaspiro[4.5]decan-1-one and (S)-7-((7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)methyl)-2,7-diazaspiro[4.5]decan-1-one

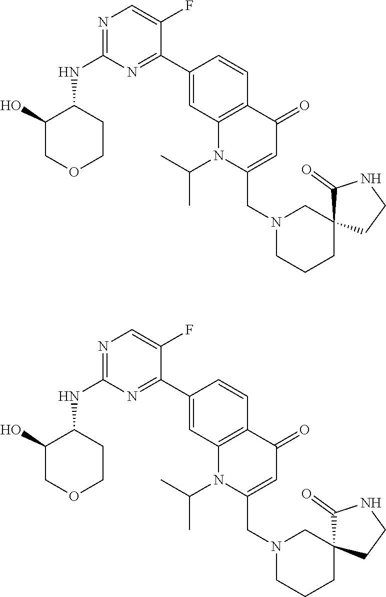 [SMILES: CC(C)n1c(CN2CCC[C@@]3(CCNC3=O)C2)cc(=O)c2ccc(-c3nc(N[C@@H]4CCOC[C@H]4O)ncc3F)cc21 ; CC(C)n1c(CN2CCC[C@]3(CCNC3=O)C2)cc(=O)c2ccc(-c3nc(N[C@@H]4CCOC[C@H]4O)ncc3F)cc21]

Mixture of Example 104 and Example 105 was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and racemic 2,7-diazaspiro[4.5]decan-1-one. Example 104 and Example 105 were separated by chiral prep-HPLC using 70% mobile phase A and 30% mobile phase B. Chiral HPLC condition: Cellulose-C column, 20.0 mm×250 mm, 5 um. Mobile phase A: hexane, Mobile phase B: ethanol containing 0.2% 2M NH₃ in methanol, 18 mL/min in 15 min.

Example 104 (25 mg, 69%). RT=9.5 min. ¹H-NMR (400 MHz, DMSO-d6) δ 8.73 (s, 1H), 8.52 (s, 1H), 8.31 (d, J=8.1 Hz, 1H), 7.94 (d, J=8.2 Hz, 1H), 7.59 (s, 1H), 7.32 (d, J=7.7 Hz, 1H), 6.16 (s, 1H), 5.53-5.34 (m, 1H), 4.97 (d, J=4.5 Hz, 1H), 3.96-3.76 (m, 3H), 3.70-3.66 (m, 1H), 3.53 (brs, 1H), 3.49-3.46 (m, 1H), 3.31 (brs, 1H), 3.13 (s, 2H), 3.07-3.01 (m, 1H), 2.82 (d, J=9.7 Hz, 1H), 2.64 (d, J=11.1 Hz, 1H), 2.15 (d, J=11.1 Hz, 1H), 2.18-2.02 (m, 2H), 1.95-1.93 (m, 2H), 1.84-1.64 (m, 6H), 1.74-1.51 (m, 3H), 1.43 (s, 2H). LC-MS (M+H)⁺=565.1.

Example 105 (24 mg, 68%). RT=11 min. ¹H-NMR (400 MHz, DMSO-d6) δ 8.73 (s, 1H), 8.52 (s, 1H), 8.31 (d, J=8.4 Hz, 1H), 7.94 (d, J=8.0 Hz, 1H), 7.59 (s, 1H), 7.32 (d, J=7.8 Hz, 1H), 6.16 (s, 1H), 5.44-5.40 (m, 1H), 4.98 (s, 1H), 3.95-3.75 (m, 3H), 3.70-3.67 (m, 1H), 3.54 (brs, 1H), 3.48-3.45 (m, 1H), 3.30 (s, 1H), 3.14 (s, 2H), 3.04 (t, J=10.1 Hz, 1H), 2.82 (d, J=9.8 Hz, 1H), 2.64 (d, J=10.8 Hz, 1H), 2.17-2.15 (m, 1H), 2.07-1.80 (m, 4H), 1.74 (t, J=7.4 Hz, 6H), 1.65-1.47 (m, 3H), 1.43 (brs, 2H). LC-MS (M+H)⁺=565.1.

### Example 106: 2-(((3R,5R)-3,5-dimethylmorpholino)methyl)-7-(5-fluoro-2-(((3S,4R)-3-hydroxytetrahydro-2H-pyran-4-yl)amino)pyrimidin-4-yl)-1-isopropylquinolin-4(1H)-one

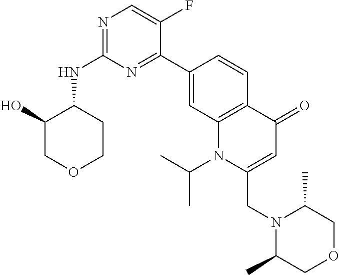 [SMILES: CC(C)n1c(CN2[C@H](C)COC[C@H]2C)cc(=O)c2ccc(-c3nc(N[C@@H]4CCOC[C@H]4O)ncc3F)cc21]

The title compound (10 mg, 50%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R,5R)-3,5-dimethylmorpholine hydrogen chloride salt. ¹H-NMR (400 MHz, DMSO-d6) δ 8.74 (brs, 1H), 8.52 (d, J=3.7 Hz, 1H), 8.30 (d, J=8.4 Hz, 1H), 7.94 (d, J=8.2 Hz, 1H), 7.32 (d, J=8.2 Hz, 1H), 6.32 (s, 1H), 5.69-5.56 (m, 1H), 4.97 (s, 1H), 4.29-4.22 (m, 1H), 3.97-3.78 (m, 3H), 3.65-3.58 (m, 2H), 3.58-3.49 (m, 1H), 3.47-3.40 (m, 1H), 3.38-3.26 (m, 3H), 3.04 (t, J=10.2 Hz, 1H), 2.81-2.71 (m, 2H), 2.06-1.96 (m, 1H), 1.80-1.68 (m, 6H), 1.59-1.46 (m, 1H), 1.08-1.02 (m, 6H). LC-MS (M+H)⁺=526.1.

### Example 107: 2-(((3R,5S)-3,5-dimethylmorpholino)methyl)-7-(5-fluoro-2-(((3S,4R)-3-hydroxytetrahydro-2H-pyran-4-yl)amino)pyrimidin-4-yl)-1-isopropylquinolin-4(1H)-one

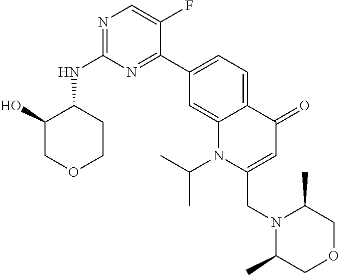 [SMILES: CC(C)n1c(CN2[C@H](C)COC[C@@H]2C)cc(=O)c2ccc(-c3nc(N[C@@H]4CCOC[C@H]4O)ncc3F)cc21]

The title compound (10 mg, 50%) was prepared in a manner similar to that in Example 18 step 2 from 2-(chloromethyl)-7-(5-fluoro-2-(((3S,4R)-3-hydroxytetrahydro-2H-pyran-4-yl)amino)pyrimidin-4-yl)-1-isopropylquinolin-4(1H)-one and (3R,5S)-3,5-dimethylmorpholine hydrogen chloride salt. ¹H-NMR (400 MHz, DMSO-d6) δ 8.67 (brs, 1H), 8.52 (s, 1H), 8.29 (d, J=8.3 Hz, 1H), 7.92 (d, J=8.2 Hz, 1H), 7.31 (d, J=7.7 Hz, 1H), 6.72 (s, 1H), 5.22-5.12 (m, 1H), 4.98 (s, 1H), 3.95-3.80 (m, 5H), 3.75-3.65 (m, 2H), 3.59-3.49 (m, 1H), 3.38-3.28 (m, 1H), 3.27-3.17 (m, 2H), 3.04 (t, J=10.0 Hz, 1H), 2.73-2.63 (s, 2H), 2.06-1.96 (m, 1H), 1.76-1.69 (m, 6H), 1.58-1.46 (m, 11H), 0.90-0.84 (m, 6H). LC-MS (M+H)⁺=526.1.

### Example 108: 2-((dimethylamino)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

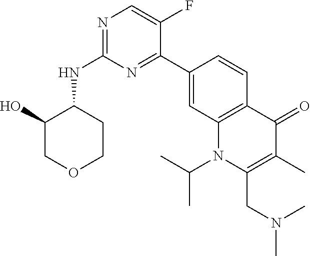 [SMILES: Cc1c(CN(C)C)n(C(C)C)c2cc(-c3nc(N[C@@H]4CCOC[C@H]4O)ncc3F)ccc2c1=O]

The title compound (15 mg, 82%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and dimethylamine. ¹H-NMR (400 MHz, DMSO-d6) δ 8.72 (brs, 1H), 8.51 (s, 1H), 8.34 (d, J=8.2 Hz, 1H), 7.90 (d, J=8.1 Hz, 1H), 7.30 (d, J=7.5 Hz, 1H), 5.60-5.48 (m, 1H), 4.97 (s, 1H), 3.95-3.80 (m, 3H), 3.70 (s, 2H), 3.60-3.50 (m, 1H), 3.38-3.28 (m, 1H), 3.04 (t, J=10.4 Hz, 1H), 2.29 (s, 6H), 2.18 (s, 3H), 2.06-1.97 (m, 1H), 1.75-1.68 (m, 6H), 1.58-1.46 (m, 1H). LC-MS (M+H)⁺=470.1.

### Example 109: 2-((cyclopropylamino)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

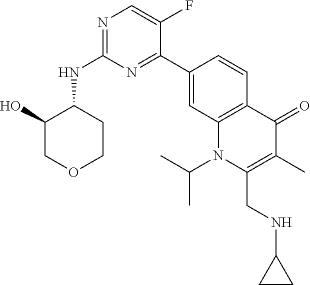 [SMILES: Cc1c(CNC2CC2)n(C(C)C)c2cc(-c3nc(N[C@@H]4CCOC[C@H]4O)ncc3F)ccc2c1=O]

The title compound (15 mg, 80%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and cyclopropanamine. ¹H-NMR (400 MHz, DMSO-d6) δ 8.66 (brs, 1H), 8.50 (s, 1H), 8.32 (d, J=8.7 Hz, 1H), 7.89 (d, J=7.9 Hz, 1H), 7.30 (d, J=7.8 Hz, 1H), 5.51-5.39 (s, 1H), 4.97 (s, 1H), 3.97-3.80 (m, 5H), 3.58-3.48 (m, 1H), 3.38-3.28 (m, 1H), 3.04 (t, J=10.3 Hz, 1H), 2.30-2.22 (m, 1H), 2.20 (s, 3H), 2.05-1.96 (m, 1H), 1.75-1.65 (m, 6H), 1.58-1.45 (m, 1H), 0.48-0.40 (m, 2H), 0.34-0.26 (m, 2H). LC-MS (M+H)⁺=482.1.

### Example 110: 7-(5-fluoro-2-(((3S,4R)-3-hydroxytetrahydro-2H-pyran-4-yl)amino)pyrimidin-4-yl)-1-isopropyl-2-((isopropylamino)methyl)-3-methylquinolin-4(1H)-one

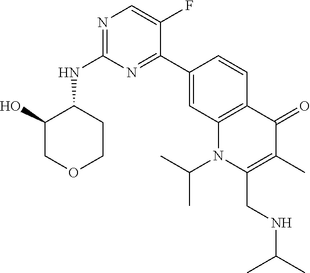 [SMILES: Cc1c(CNC(C)C)n(C(C)C)c2cc(-c3nc(N[C@@H]4CCOC[C@H]4O)ncc3F)ccc2c1=O]

The title compound (15 mg, 79%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and propan-2-amine. ¹H-NMR (400 MHz, DMSO-d6) δ 8.67 (brs, 1H), 8.51 (s, 1H), 8.32 (d, J=8.1 Hz, 1H), 7.89 (d, J=8.7 Hz, 1H), 7.30 (d, J=9.1 Hz, 1H), 5.62-5.50 (m, 1H), 4.98 (s, 1H), 3.94-3.80 (m, 5H), 3.59-3.49 (m, 1H), 3.38-3.29 (m, 1H), 3.04 (t, J=10.7 Hz, 1H), 2.93-2.83 (m, 1H), 2.17 (s, 3H), 2.05-1.96 (m, 1H), 1.77-1.67 (s, 6H), 1.60-1.38 (m, 2H), 1.10 (brs, 6H). LC-MS (M+H)⁺=484.1.

### Example 111: 7-(5-fluoro-2-(((3S,4R)-3-hydroxytetrahydro-2H-pyran-4-yl)amino)pyrimidin-4-yl)-2-((((1r,3R)-3-fluorocyclobutyl)amino)methyl)-1-isopropyl-3-methylquinolin-4(1H)-one

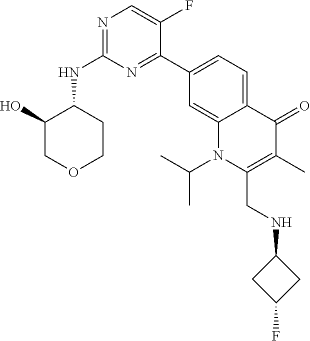 [SMILES: Cc1c(CN[C@H]2C[C@H](F)C2)n(C(C)C)c2cc(-c3nc(N[C@@H]4CCOC[C@H]4O)ncc3F)ccc2c1=O]

The title compound (13 mg, 65%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (1r,3r)-3-fluorocyclobutan-1-amine. ¹H-NMR (400 MHz, DMSO-d6) δ 8.68 (brs, 1H), 8.51 (d. J=2.1 Hz, 1H), 8.32 (d, J=8.2 Hz, 1H), 7.89 (d, J=8.3 Hz, 1H), 7.30 (d, J=7.7 Hz, 1H), 5.56-5.46 (m, 1H), 5.35-5.12 (m, 1H), 4.97 (d, J=3.8 Hz, 1H), 3.95-3.73 (m, 5H), 3.58-3.48 (m, 2H), 3.38-3.29 (m, 1H), 3.04 (t, J=10.4 Hz, 1H), 2.80-2.70 (m, 1H), 2.42-2.13 (m, 4H), 2.16 (s, 3H), 2.06-1.96 (m, 11H), 1.78-1.69 (m, 6H), 1.59-1.46 (m, 1H). LC-MS (M+H)⁺=514.1.

### Example 112: 7-(5-fluoro-2-(((3S,4R)-3-hydroxytetrahydro-2H-pyran-4-yl)amino)pyrimidin-4-yl)-1-isopropyl-3-methyl-2-((((R)-tetrahydrofuran-3-yl)amino)methyl)quinolin-4(1H)-one

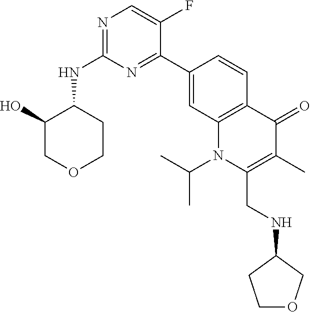 [SMILES: Cc1c(CN[C@@H]2CCOC2)n(C(C)C)c2cc(-c3nc(N[C@@H]4CCOC[C@H]4O)ncc3F)ccc2c1=O]

The title compound (10 mg, 50%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (R)-tetrahydrofuran-3-amine. ¹H-NMR (400 MHz, DMSO-d6) δ 8.68 (brs, 1H), 8.51 (s, 1H), 8.32 (d, J=8.5 Hz, 1H), 7.89 (d, J=8.4 Hz, 1H), 7.30 (d, J=8.0 Hz, 1H), 5.62-5.46 (m, 1H), 4.97 (d, J=4.6 Hz, 1H), 3.95-3.76 (m, 7H), 3.75-3.67 (m, 1H), 3.58-3.50 (m, 2H), 3.48-3.41 (m, 1H), 3.37-3.29 (m, 1H), 3.04 (t, J=10.4 Hz, 1H), 2.18 (s, 3H), 2.06-1.96 (m, 2H), 1.85-1.65 (m, 7H), 1.60-1.45 (m, 1H). LC-MS (M+H)⁺=512.1.

### Example 113: 7-(5-fluoro-2-(((3S,4R)-3-hydroxytetrahydro-2H-pyran-4-yl)amino)pyrimidin-4-yl)-1-isopropyl-3-methyl-2-((((S)-tetrahydrofuran-3-yl)amino)methyl)quinolin-4(1H)-one

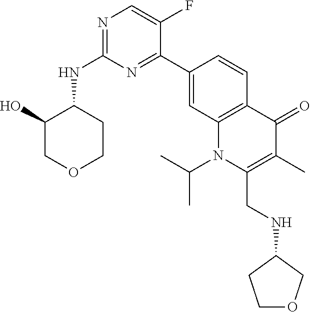 [SMILES: Cc1c(CN[C@H]2CCOC2)n(C(C)C)c2cc(-c3nc(N[C@@H]4CCOC[C@H]4O)ncc3F)ccc2c1=O]

The title compound (10 mg, 50%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (S)-tetrahydrofuran-3-amine. ¹H-NMR (400 MHz, DMSO-d6) δ 8.68 (brs, 1H), 8.51 (s, 1H), 8.32 (d, J=8.5 Hz, 1H), 7.89 (d, J=8.4 Hz, 1H), 7.30 (d, J=8.0 Hz, 1H), 5.62-5.46 (m, 1H), 4.97 (d, J=4.6 Hz, 1H), 3.95-3.76 (m, 7H), 3.75-3.67 (m, 1H), 3.58-3.50 (m, 2H), 3.48-3.41 (m, 1H), 3.37-3.29 (m, 1H), 3.04 (t, J=10.4 Hz, 1H), 2.18 (s, 3H), 2.06-1.96 (m, 2H), 1.85-1.65 (m, 7H), 1.60-1.45 (m, 1H). LC-MS (M+H)⁺=512.1.

### Example 114: 6-fluoro-7-(5-fluoro-2-(((3S,4R)-3-hydroxytetrahydro-2H-pyran-4-yl)amino)pyrimidin-4-yl)-2-(((R)-3-fluoropyrrolidin-1-yl)methyl)-1-isopropylquinolin-4(1H)-one

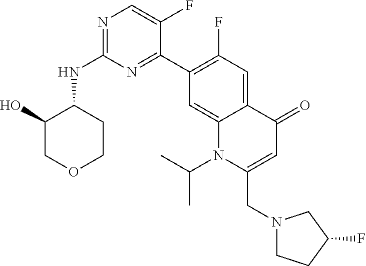 [SMILES: CC(C)n1c(CN2CC[C@@H](F)C2)cc(=O)c2cc(F)c(-c3nc(N[C@@H]4CCOC[C@H]4O)ncc3F)cc21]

### Step 1: (E)-1-(4-bromo-2,5-difluorophenyl)-3-(dimethylamino)but-2-en-1-one

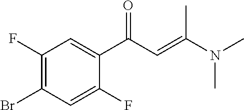 [SMILES: C/C(=C\C(=O)c1cc(F)c(Br)cc1F)N(C)C]

The title compound (730 mg. 51%) was prepared in a manner similar to that in Example 6 step 1 from 1-(4-bromo-2,5-difluorophenyl)ethan-1-one and 1,1-dimethoxy-N,N-dimethylethan-1-amine. LC-MS (M+H)⁺=303.9.

### Step 2: (E)-1-(4-bromo-2,5-difluorophenyl)-3-(isopropylamino)but-2-en-1-one

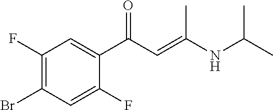 [SMILES: C/C(=C\C(=O)c1cc(F)c(Br)cc1F)NC(C)C]

The title compound (600 mg, 77%) was prepared in a manner similar to that in Example 6 step 2 from (E)-1-(4-bromo-2,5-difluorophenyl)-3-(dimethylamino)but-2-en-1-one and propan-2-amine. LC-MS (M+H)⁺=317.9.

### Step 3: 7-bromo-6-fluoro-1-isopropyl-2-methylquinolin-4(1H)-one

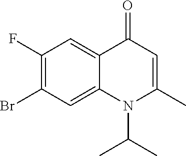 [SMILES: Cc1cc(=O)c2cc(F)c(Br)cc2n1C(C)C]

The title compound (450 mg, 80%) was prepared in a manner similar to that in Example 6 step 3 from (E)-1-(4-bromo-2,5-difluorophenyl)-3-(isopropylamino)but-2-en-1-one. LC-MS (M+H)⁺=297.9.

### Step 4: 6-fluoro-1-isopropyl-2-methyl-7-(4,4,5,5-tetramethyl-1,3,2-dioxaborolan-2-yl)quinolin-4(1H)-one

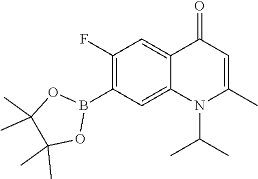

The title compound (300 mg. 75%) was prepared in a manner similar to that in Example 1 step 6 from 7-bromo-6-fluoro-1-isopropyl-2-methylquinolin-4(1H)-one and bis(pinacolato)diboron. LC-MS (M+H)⁺=264.1 for corresponding boronic acid.

### Step 5: (3S,4R)-4-((5-fluoro-4-(6-fluoro-1-isopropyl-2-methyl-4-oxo-1,4-dihydroquinolin-7-yl)pyrimidin-2-yl)amino)tetrahydro-2H-pyran-3-yl acetate

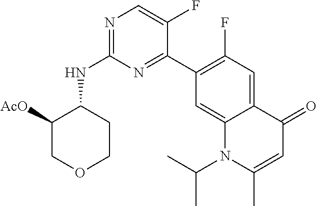 [SMILES: CC(=O)O[C@@H]1COCC[C@H]1Nc1ncc(F)c(-c2cc3c(cc2F)c(=O)cc(C)n3C(C)C)n1]

The title compound (200 mg. 31%) was prepared in a manner similar to that in Example 1 step 7 from 6-fluoro-1-isopropyl-2-methyl-7-(4,4,5,5-tetramethyl-1,3,2-dioxaborolan-2-yl)quinolin-4(1H)-one and (3S,4R)-4-((4-chloro-5-fluoropyrimidin-2-yl)amino)tetrahydro-2H-pyran-3-yl acetate. LC-MS (M+H)⁺=473.2.

### Step 6: (3S,4R)-4-((5-fluoro-4-(6-fluoro-2-formyl-1-isopropyl-4-oxo-1,4-dihydroquinolin-7-yl)pyrimidin-2-yl)amino)tetrahydro-2H-pyran-3-yl acetate

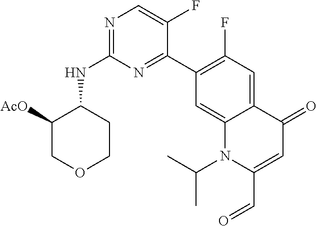 [SMILES: CC(=O)O[C@@H]1COCC[C@H]1Nc1ncc(F)c(-c2cc3c(cc2F)c(=O)cc(C=O)n3C(C)C)n1]

The title compound (56 mg, 27%) was prepared in a manner similar to that in Example 17 step 1 from (3S,4R)-4-((5-fluoro-4-(6-fluoro-1-isopropyl-2-methyl-4-oxo-1,4-dihydroquinolin-7-yl)pyrimidin-2-yl)amino)tetrahydro-2H-pyran-3-yl acetate. LC-MS (M+H)⁺=487.1.

### Step 7: (3S,4R)-4-((5-fluoro-4-(6-fluoro-2-(((R)-3-fluoropyrrolidin-1-yl)methyl)-1-isopropyl-4-oxo-1,4-dihydroquinolin-7-yl)pyrimidin-2-yl)amino)tetrahydro-2H-pyran-3-yl acetate

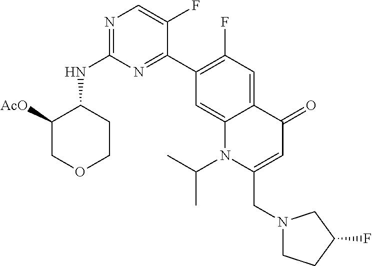 [SMILES: CC(=O)O[C@@H]1COCC[C@H]1Nc1ncc(F)c(-c2cc3c(cc2F)c(=O)cc(CN2CC[C@@H](F)C2)n3C(C)C)n1]

The title compound (57 mg, 88%) was prepared in a manner similar to that in Example 37 step 6 from (3S,4R)-4-((5-fluoro-4-(6-fluoro-2-formyl-1-isopropyl-4-oxo-1,4-dihydroquinolin-7-yl)pyrimidin-2-yl)amino)tetrahydro-2H-pyran-3-yl acetate and (R)-3-fluoropyrrolidine. LC-MS (M+H)⁺=560.3.

### Step 8: 6-fluoro-7-(5-fluoro-2-(((3S,4R)-3-hydroxytetrahydro-2H-pyran-4-yl)amino)pyrimidin-4-yl)-2-(((R)-3-fluoropyrrolidin-1-yl)methyl)-1-isopropylquinolin-4(1H)-one

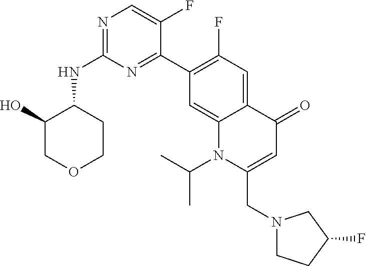 [SMILES: CC(C)n1c(CN2CC[C@@H](F)C2)cc(=O)c2cc(F)c(-c3nc(N[C@@H]4CCOC[C@H]4O)ncc3F)cc21]

The title compound (12 mg, 23%) was prepared in a manner similar to that in Example 55 step 10 from (3S,4R)-4-((5-fluoro-4-(6-fluoro-2-(((R)-3-fluoropyrrolidin-1-yl)methyl)-1-isopropyl-4-oxo-1,4-dihydroquinolin-7-yl)pyrimidin-2-yl)amino)tetrahydro-2H-pyran-3-yl acetate. ¹H-NMR (400 MHz, DMSO-d6) δ 8.52 (d, J=2.3 Hz, 1H), 8.28 (brs, 1H), 7.97 (d, J=10.1 Hz, 1H), 7.40 (d, J=7.8 Hz, 1H), 6.24 (s, 1H), 5.41-5.26 (m, 2H), 4.96 (d, J=5.2 Hz, 1H), 3.87-3.74 (m, 5H), 3.58-3.46 (m, 1H), 3.32-3.21 (m, 1H), 3.04-2.95 (m, 1H), 2.92-2.81 (m, 2H), 2.76-2.60 (m, 1H), 2.45-2.35 (m, 1H), 2.27-2.08 (m, 1H), 2.04-1.80 (m, 2H), 1.70-1.60 (m, 6H), 1.58-1.43 (m, 1H). LC-MS (M+H)⁺=518.3.

### Example 115: 7-(5-fluoro-2-(((3S,4R)-3-hydroxytetrahydro-2H-pyran-4-yl)amino)pyrimidin-4-yl)-2-((4-hydroxypiperidin-1-yl)methyl)-1-isopropyl-3-methylquinolin-4(1H)-one

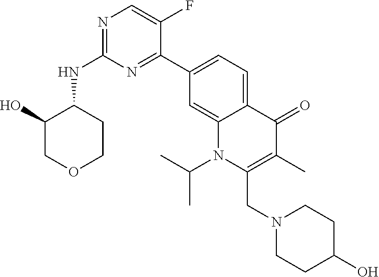 [SMILES: Cc1c(CN2CCC(O)CC2)n(C(C)C)c2cc(-c3nc(N[C@@H]4CCOC[C@H]4O)ncc3F)ccc2c1=O]

The title compound (22 mg, 55%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and piperidin-4-ol. ¹H-NMR (400 MHz, DMSO-d6) δ 8.73 (brs, 1H), 8.51 (d, J=3.8 Hz, 1H), 8.34 (d, J=8.5 Hz, 1H), 7.95-7.86 (m, 1H), 7.29 (d, J=7.9 Hz, 1H), 5.56-5.40 (m, 1H), 4.96 (d, J=5.2 Hz, 1H), 4.59 (d, J=4.1 Hz, 1H), 3.97-3.80 (m, 3H), 3.77 (s, 2H), 3.63-3.46 (m, 2H), 3.41-3.27 (m, 1H), 3.11-2.98 (m, 1H), 2.81-2.71 (m, 2H), 2.31-2.19 (m, 2H), 2.17 (s, 3H), 2.08-1.98 (m, 1H), 1.79-1.69 (m, 8H), 1.65-1.45 (m, 1H), 1.43-1.34 (m, 2H). LC-MS (M+H)⁺=526.2.

### Example 116: 2-((3,3-difluoropiperidin-1-yl)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

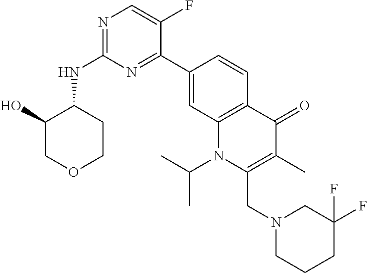 [SMILES: Cc1c(CN2CCCC(F)(F)C2)n(C(C)C)c2cc(-c3nc(N[C@@H]4CCOC[C@H]4O)ncc3F)ccc2c1=O]

The title compound (8 mg, 37%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 3,3-difluoropiperidine. ¹H-NMR (400 MHz, DMSO-d6) δ 8.74 (brs, 1H), 8.51 (d, J=2.9 Hz, 1H), 8.35 (d, J=8.6 Hz, 1H), 7.91 (d, J=8.4 Hz, 1H), 7.31 (d, J=7.8 Hz, 1H), 5.46-5.33 (m, 1H), 4.97 (d, J=4.5 Hz, 1H), 3.95-3.80 (m, 5H), 3.58-3.48 (m, 1H), 3.38-3.28 (m, 1H), 3.03 (t, J=10.4 Hz, 1H), 2.89-2.79 (m, 2H), 2.58-2.50 (m, 2H), 2.18 (s, 3H), 2.05-1.84 (m, 3H), 1.77-1.60 (m, 8H), 1.58-1.46 (m, 1H). LC-MS (M+H)⁺=546.1.

### Example 117: 2-((7-oxa-4-azaspiro[2.5]octan-4-yl)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

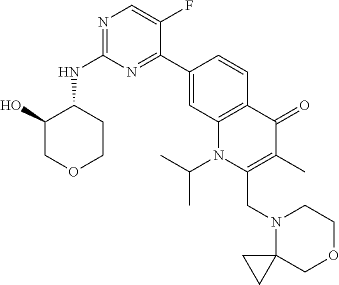 [SMILES: Cc1c(CN2CCOCC23CC3)n(C(C)C)c2cc(-c3nc(N[C@@H]4CCOC[C@H]4O)ncc3F)ccc2c1=O]

The title compound (3 mg, 14%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 7-oxa-4-azaspiro[2.5]octane. ¹H-NMR (400 MHz, DMSO-d6) δ 8.71 (brs, 1H), 8.51 (d, J=3.1 Hz, 1H), 8.33 (d, J=8.5 Hz, 1H), 7.90 (d, J=8.4 Hz, 1H), 7.30 (d, J=7.8 Hz, 1H), 5.36-5.23 (m, 1H), 4.97 (s, 1H), 4.05 (s, 2H), 3.96-3.79 (m, 3H), 3.69-3.63 (m, 2H), 3.58-3.49 (m, 3H), 3.39-3.29 (m, 1H), 3.04 (t, J=10.3 Hz, 1H), 2.81-2.75 (m, 2H), 2.21 (s, 3H), 2.06-1.96 (m, 1H), 1.75-1.64 (m, 6H), 1.59-1.44 (m, 1H), 0.77-0.70 (m, 2H), 0.53-0.46 (m, 2H). LC-MS (M+H)⁺=538.1.

### Example 118: 2-(((3S,5S)-3,5-dimethylmorpholino)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

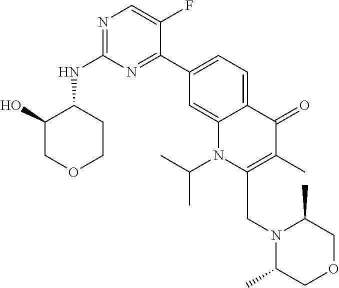 [SMILES: Cc1c(CN2[C@@H](C)COC[C@@H]2C)n(C(C)C)c2cc(-c3nc(N[C@@H]4CCOC[C@H]4O)ncc3F)ccc2c1=O]

The title compound (I mg, 5%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (3S,5S)-3,5-dimethylmorpholine hydrochloride. ¹H-NMR (400 MHz, DMSO-d6) δ 8.74 (brs, 1H), 8.51 (d, J=2.7 Hz, 1H), 8.32 (d, J=8.2 Hz, 1H), 7.90 (d, J=8.3 Hz, 1H), 7.31 (d, J=7.6 Hz, 1H), 5.67-5.51 (m, 1H), 4.99 (s, 1H), 4.22-4.15 (m, 1H), 3.96-3.80 (m, 5H), 3.66-3.49 (m, 4H), 3.38-3.28 (m, 1H), 3.04 (t, J=10.4 Hz, 1H), 2.86-2.76 (m, 2H), 2.24 (s, 3H), 2.06-1.96 (m, 1H), 1.82-1.69 (m, 6H), 1.59-1.46 (m, 1H), 1.05-0.98 (m, 6H). LC-MS (M+H)⁺=540.1.

### Example 119: 7-(5-fluoro-2-(((3S,4R)-3-hydroxytetrahydro-2H-pyran-4-yl)amino)pyrimidin-4-yl)-2-(hydroxymethyl)-1-isopropyl-3-methylquinolin-4(1H)-one

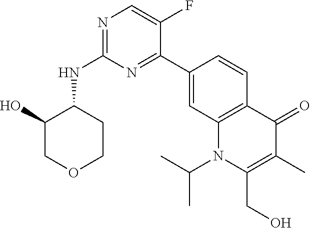 [SMILES: Cc1c(CO)n(C(C)C)c2cc(-c3nc(N[C@@H]4CCOC[C@H]4O)ncc3F)ccc2c1=O]

The title compound (10 mg, 56%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and water. ¹H-NMR (400 MHz, DMSO-d6) δ 8.67 (brs, 1H), 8.51 (s, 11H), 8.33 (d, J=8.4 Hz, 1H), 7.90 (d, J=8.2 Hz, 1H), 7.30 (d, J=7.7 Hz, 1H), 5.79 (brs, 1H), 5.45-5.25 (m, 1H), 4.98 (brs, 1H), 4.71 (s, 2H), 3.95-3.80 (m, 3H), 3.57-3.50 (m, 1H), 3.38-3.28 (m, 1H), 3.04 (t, J=10.1 Hz, 1H), 2.18 (s, 3H), 2.07-1.97 (m, 1H), 1.78-1.70 (m, 6H), 1.60-1.45 (m, 1H). LC-MS (M+H)⁺=443.1.

### Example 120: 2-(aminomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

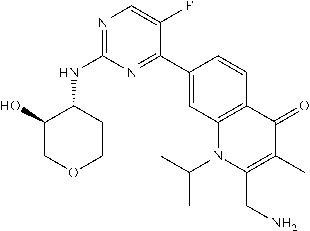 [SMILES: Cc1c(CN)n(C(C)C)c2cc(-c3nc(N[C@@H]4CCOC[C@H]4O)ncc3F)ccc2c1=O]

The title compound (10 mg, 56%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and ammonia. ¹H-NMR (400 MHz, DMSO-d6) δ 8.66 (brs, 1H), 8.52 (s, 1H), 8.32 (d, J=8.6 Hz, 1H), 7.91 (d, J=8.2 Hz, 1H), 7.32 (d, J=7.5 Hz, 1H), 6.86 (brs, 2H), 5.32-5.20 (m, 1H), 5.01 (d, J=4.6 Hz, 1H), 4.12 (s, 2H), 3.95-3.80 (m, 3H), 3.60-3.50 (m, 1H), 3.38-3.28 (m, 1H), 3.04 (t, J=10.2 Hz, 1H), 2.18 (s, 3H), 2.06-1.96 (m, 1H), 1.78-1.70 m, 6H), 1.59-1.46 (m, 1H). LC-MS (M+H)⁺=442.1.

### Example 121: 2-((4,4-difluoropiperidin-1-yl)methyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

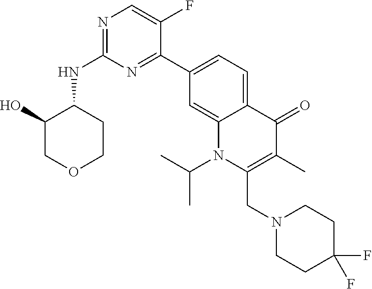 [SMILES: Cc1c(CN2CCC(F)(F)CC2)n(C(C)C)c2cc(-c3nc(N[C@@H]4CCOC[C@H]4O)ncc3F)ccc2c1=O]

The title compound (1.5 mg, 7%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 4,4-difluoropiperidine hydrochloride. ¹H-NMR (400 MHz, DMSO-d6) δ 8.76 (s, 1H), 8.51 (s, 1H), 8.34 (d, J=8.2 Hz, 1H), 7.91 (d, J=8.5 Hz, 1H), 7.31 (d, J=7.5 Hz, 1H), 5.56-5.40 (m, 1H), 4.97 (s, 1H), 4.02-3.76 (m, 5H), 3.54 (s, 1H), 3.04 (t, J=10.3 Hz, 11H), 2.65 (s, 4H), 2.54 (s, 1H), 2.17 (s, 3H), 2.10-1.89 (m, 5H), 1.75 (t, J=7.1 Hz, 61H), 1.64-1.44 (m, J=11.9 Hz, 1H). LC-MS (M+H)⁺=546.1.

### Example 122: 7-(5-fluoro-2-(((3S,4R)-3-hydroxytetrahydro-2H-pyran-4-yl)amino)pyrimidin-4-yl)-2-(((R)-3-hydroxypiperidin-1-yl)methyl)-1-isopropyl-3-methylquinolin-4(1H)-one

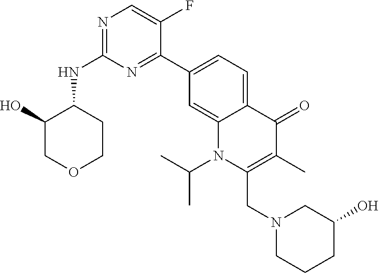 [SMILES: Cc1c(CN2CCC[C@@H](O)C2)n(C(C)C)c2cc(-c3nc(N[C@@H]4CCOC[C@H]4O)ncc3F)ccc2c1=O]

The title compound (5.1 mg, 23%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (R)-piperidin-3-ol hydrochloride. ¹H-NMR (400 MHz, DMSO-d6) δ 8.73 (s, 1H), 8.51 (s, 1H), 8.34 (d, J=8.2 Hz, 1H), 7.91 (d, J=8.3 Hz, 1H), 7.30 (d, J=7.8 Hz, 1H), 5.47 (d, J=7.1 Hz, 1H), 4.97 (d, J=4.7 Hz, 1H), 4.64 (d, J=3.7 Hz, 1H), 4.02-3.72 (m, 5H), 3.59-3.49 (m, 1H), 3.43 (s, 1H), 3.35 (s, 1H), 3.32-3.28 (m, 1H), 3.04 (t, J=10.4 Hz, 1H), 2.91-2.82 (m, 1H), 2.71 (s, 1H), 2.17 (s, 3H), 2.15-1.90 (m, J=35.7, 23.0 Hz, 3H), 1.87-1.57 (m, 1H), 1.77-1.70 (m, 6H), 1.59-1.47 (m, J=10.9 Hz, 1H), 1.45-1.31 (m, 1H), 1.19-1.04 (m, J=11.1 Hz, 1H). LC-MS (M+H)⁺=526.1.

### Example 123: 7-(5-fluoro-2-(((3S,4R)-3-hydroxytetrahydro-2H-pyran-4-yl)amino)pyrimidin-4-yl)-2-(((S)-3-hydroxypiperidin-1-yl)methyl)-1-isopropyl-3-methylquinolin-4(1H)-one

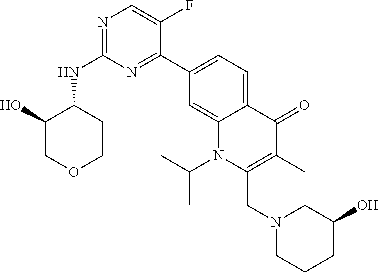 [SMILES: Cc1c(CN2CCC[C@H](O)C2)n(C(C)C)c2cc(-c3nc(N[C@@H]4CCOC[C@H]4O)ncc3F)ccc2c1=O]

The title compound (5 mg, 23%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (S)-piperidin-3-ol hydochloride. ¹H-NMR (400 MHz, DMSO-d6) δ 8.73 (s, 1H), 8.51 (s, 1H), 8.34 (d, J=8.2 Hz, 1H), 7.91 (d, J=8.3 Hz, 1H), 7.30 (d, J=7.8 Hz, 1H), 5.57-5.37 (m, 1H), 4.97 (d, J=4.7 Hz, 1H), 4.64 (d, J=3.7 Hz, 1H), 4.02-3.72 (m, 5H), 3.59-3.49 (m, 1H), 3.43 (s, 1H), 3.35 (s, 11H), 3.32-3.28 (m, 1H), 3.04 (t, J=10.4 Hz, 1H), 2.91-2.82 (m, 1H), 2.71 (s, 1H), 2.17 (s, 3H), 2.15-1.90 (m, J=35.7, 23.0 Hz, 3H), 1.87-1.57 (m, 11H), 1.77-1.70 (m, 6H), 1.59-1.47 (m, J=10.9 Hz, 1H), 1.45-1.31 (m, 1H), 1.19-1.04 (m, J=11.1 Hz, 1H). LC-MS (M+H)⁺=526.1.

### Example 124: 7-((7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinolin-2-yl)methyl)-2,7-diazaspiro[4.5]decan-1-one

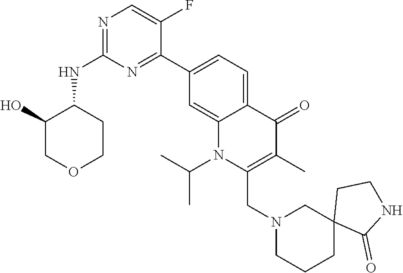 [SMILES: Cc1c(CN2CCCC3(CCNC3=O)C2)n(C(C)C)c2cc(-c3nc(N[C@@H]4CCOC[C@H]4O)ncc3F)ccc2c1=O]

The title compound (4 mg, 20%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and 2,7-diazaspiro[4.5]decan-1-one. ¹H-NMR (400 MHz, DMSO-d6) δ 8.72 (brs, 1H), 8.51 (s, 1H), 8.34 (d, J=8.4 Hz, 1H), 7.90 (d, J=7.9 Hz, 1H), 7.58 (s, 1H), 7.30 (d, J=7.8 Hz, 1H), 5.65-5.51 (m, 1H), 4.97 (d, J=4.7 Hz, 1H), 3.95-3.75 (m, 3H), 3.74 (s, 2H), 3.59-3.49 (m, 1H), 3.38-3.29 (m, 1H), 3.17-3.09 (m, 2H), 3.04 (t, J=10.4 Hz, 1H), 2.82 (d. J=10.2 Hz, 1H), 2.69-2.60 (m, 1H), 2.34-2.26 (m, 1H), 2.16 (s, 3H), 2.14-1.88 (m, 4H), 1.78-1.70 m, 6H), 1.65-1.38 (m, 5H). LC-MS (M+H)⁺=579.2.

### Example 125: 7-(5-fluoro-2-(((3S,4R)-3-hydroxytetrahydro-2H-pyran-4-yl)amino)pyrimidin-4-yl)-2-(((S)-3-hydroxy-3-methylpyrrolidin-1-yl)methyl)-1-isopopyl-3-methylquinolin-4(1H)-one

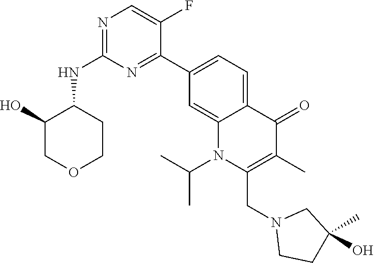 [SMILES: Cc1c(CN2CC[C@](C)(O)C2)n(C(C)C)c2cc(-c3nc(N[C@@H]4CCOC[C@H]4O)ncc3F)ccc2c1=O]

The title compound (6.9 mg, 27%) was prepared in a manner similar to that in Example 18 step 2 from 2-(bromomethyl)-7-(5-fluoro-2-(((3S,4R)-3-hydroxytetrahydro-2H-pyran-4-yl)amino)pyrimidin-4-yl)-1-isopropyl-3-methylquinolin-4(1H)-one and (S)-3-methylpyrrolidin-3-ol. ¹H-NMR (400 MHz, DMSO-d6) δ: 8.72 (s, 1H), 8.51 (s, 1H), 8.33 (d, J=7.9 Hz, 1H), 7.90 (d, J=8.1 Hz, 1H), 7.29 (d, J=7.5 Hz, 1H), 5.61-5.50 (m, 1H), 5.00-4.93 (m, 1H), 4.58 (s, 1H), 3.96-3.80 (m, 5H), 3.58-3.49 (m, 1H), 3.40-3.34 (m, 1H), 3.04 (t, J=10.3 Hz, 1H), 2.76-2.69 (m, 2H), 2.64-2.58 (m, 1H), 2.57-2.53 (m, 1H), 2.19 (s, 3H), 2.06-1.97 (m, 1H) 1.80-1.66 (m, 8H), 1.59-1.46 (m, 1H), 1.25 (s, 3H). LC-MS (M+H)⁺=526.1.

### Example 386: 2-(azetidin-2-yl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

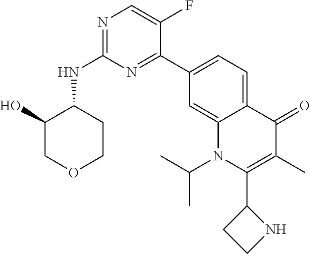 [SMILES: Cc1c(C2CCN2)n(C(C)C)c2cc(-c3nc(N[C@@H]4CCOC[C@H]4O)ncc3F)ccc2c1=O]

### Step 1: tert-butyl 2-(3-(4-chloro-2-fluorophenyl)-3-oxoprop-1-yn-1-yl)azetidine-1-carboxylate

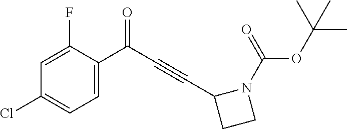 [SMILES: CC(C)(C)OC(=O)N1CCC1C#CC(=O)c1ccc(Cl)cc1F]

To a solution of 4-chloro-2-fluoro-benzoyl chloride (7 g, 36.3 mmol), copper(I) iodide (345 mg, 1.81 mmol) and bis(triphenylphosphine)palladium(II) dichloride (2.55 g, 3.63 mmol) in tetrahydrofuran (70 mL) was added dropwise triethylamine (4.77 g, 47.2 mmol, 6.56 mL) and tert-butyl 2-ethynylazetidine-1-carboxylate (6.57 g, 36.3 mmol) at 25° C. under nitrogen atmosphere. The resulting mixture was stirred at 25° C. for 12 h before addition of water (70 mL). The mixture was extracted with dichloromethane (100 mL×3). The combined organic phases were washed with brine, dried over sodium sulfate, filtered and concentrated in vacuum. The residue was purified over silica gel by column chromatography, eluting with ethyl acetate in petroleum ether (16%, v/v) to give the title compound (7.2 g, 59%). LC-MS (M+H)⁺=238.1 (M-99).

### Step 2: tert-butyl 2-(3-(4-chloro-2-fluorophenyl)-1-(isopropylamino)-3-oxoprop-1-en-1-yl)azetidine-1-carboxylate

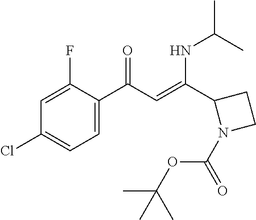 [SMILES: CC(C)N/C(=C\C(=O)c1ccc(Cl)cc1F)C1CCN1C(=O)OC(C)(C)C]

To a solution of tert-butyl 2-(3-(4-chloro-2-fluorophenyl)-3-oxoprop-1-yn-1-yl)azetidine-1-carboxylate (3 g, 8.88 mmol) in tetrahydrofuran (30 mL) and ethanol (30 mL) was added propan-2-amine (788 mg, 13.3 mmol) at 25° C. and the reaction solution was stirred for 8 h before concentration. The residue was purified over silica gel by column chromatography, eluting with ethyl acetate in petroleum ether (0-100% gradient, v/v) to give the title compound (2.5 g, 71%). LC-MS (M+H)⁺=397.2.

### Step 3: tert-butyl 2-(7-chloro-1-isopropyl-4-oxo-1,4-dihydroquinolin-2-yl)azetidine-1-carboxylate

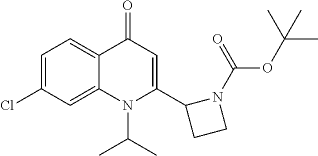 [SMILES: CC(C)n1c(C2CCN2C(=O)OC(C)(C)C)cc(=O)c2ccc(Cl)cc21]

To a solution of tert-butyl 2-(3-(4-chloro-2-fluorophenyl)-1-(isopropylamino)-3-oxoprop-1-en-1-yl)azetidine-1-carboxylate (5 g, 12.60 mmol) in dimethylacetamide (50 mL) was added potassium carbonate (2.61 g, 18.9 mmol) at 25° C. for 0.5 h under nitrogen atomosphere. The mixture was stirred at 130° C. for 48 h before cooled to room temperature and concentrated. The residue was purified over silica gel by column chromatography, eluting with ethyl acetate in petroleum ether (0-2% gradient, v/v) to give the title compound (2.1 g, 44%). LC-MS (M+H)⁺=377.2.

### Step 4: tert-butyl 2-(7-chloro-3-iodo-1-isopropyl-4-oxo-1,4-dihydroquinolin-2-yl)azetidine-1-carboxylate

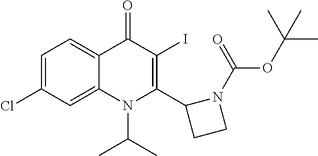 [SMILES: CC(C)n1c(C2CCN2C(=O)OC(C)(C)C)c(I)c(=O)c2ccc(Cl)cc21]

The title compound (0.8 g, 40%) was prepared in a manner similar to Example 16 step 1 from tert-butyl 2-(7-chloro-1-isopropyl-4-oxo-1,4-dihydroquinolin-2-yl)azetidine-1-carboxylate and iodine. LC-MS (M+H)⁺=503.1.

### Step 5: tert-butyl 2-(7-chloro-1-isopropyl-3-methyl-4-oxo-1,4-dihydroquinolin-2-yl)azetidine-1-carboxylate

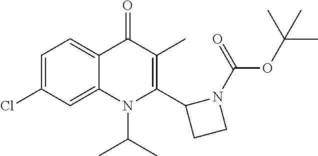 [SMILES: Cc1c(C2CCN2C(=O)OC(C)(C)C)n(C(C)C)c2cc(Cl)ccc2c1=O]

The title compound (0.52 g, 82%) was prepared in a manner similar to Example 9 step 2 from tert-butyl 2-(7-chloro-3-iodo-1-isopropyl-4-oxo-1,4-dihydroquinolin-2-yl)azetidine-1-carboxylate . LC-MS (M+H)⁺=391.1.

### Step 6: tert-butyl 2-(1-isopropyl-3-methyl-4-oxo-7-(4,4,5,5-tetramethyl-1,3,2-dioxaborolan-2-yl)-1,4-dihydroquinolin-2-yl)azetidine-1-carboxylate

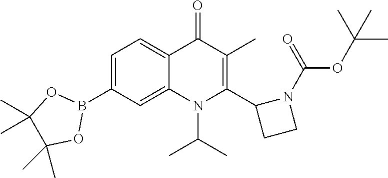 [SMILES: Cc1c(C2CCN2C(=O)OC(C)(C)C)n(C(C)C)c2cc(B3OC(C)(C)C(C)(C)O3)ccc2c1=O]

The title compound (0.5 g, 90%) was prepared in a manner similar to Example 1 step 6 from tert-butyl 2-(7-chloro-1-isopropyl-3-methyl-4-oxo-1,4-dihydroquinolin-2-yl)azetidine-1-carboxylate and 4,4,5,5-tetramethyl-2-(4,4,5,5-tetramethyl-1,3,2-dioxaborolan-2-yl)-1,3,2-dioxaborolane. LC-MS (M+H)⁺=483.4.

### Step 7: tert-butyl 2-(7-(2-chloro-5-fluoropyrimidin-4-yl)-1-isopropyl-3-methyl-4-oxo-1,4-dihydroquinolin-2-yl)azetidine-1-carboxylate

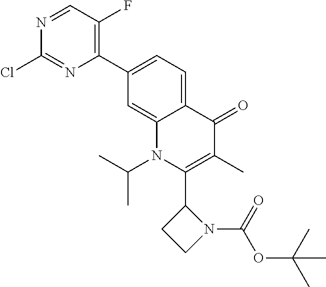 [SMILES: Cc1c(C2CCN2C(=O)OC(C)(C)C)n(C(C)C)c2cc(-c3nc(Cl)ncc3F)ccc2c1=O]

The title compound (0.5 g, 90%) was prepared in a manner similar to Example 1 step 7 from tert-butyl 2-(1-isopropyl-3-methyl-4-oxo-7-(4,4,5,5-tetramethyl-1,3,2-dioxaborolan-2-yl)-1,4-dihydroquinolin-2-yl)azetidine-1-carboxylate and 2,4-dichloro-5-fluoro-pyrimidine. LC-MS (M+H)⁺=487.2.

### Step 8: tert-butyl 2-(7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinolin-2-yl)azetidine-1-carboxylate

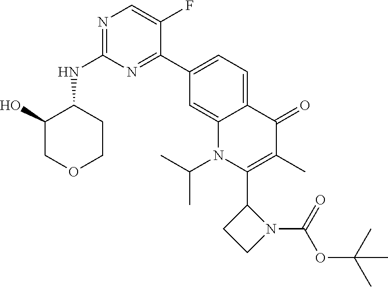 [SMILES: Cc1c(C2CCN2C(=O)OC(C)(C)C)n(C(C)C)c2cc(-c3nc(N[C@@H]4CCOC[C@H]4O)ncc3F)ccc2c1=O]

The title compound (0.15 g, 64%) was prepared in a manner similar to Example 1 step 8 from tert-butyl 2-(7-(2-chloro-5-fluoropyrimidin-4-yl)-1-isopropyl-3-methyl-4-oxo-1,4-dihydroquinolin-2-yl)azetidine-1-carboxylate and (3S,4R)-4-aminotetrahydropyran-3-ol. LC-MS (M+H)⁺=568.3.

### Step 9: 2-(azetidin-2-yl)-7-(5-fluoro-2-(((3S,4R)-3-hydroxytetrahydro-2H-pyran-4-yl)amino)pyrimidin-4-yl)-1-isopropyl-3-methylquinolin-4(1H)-one

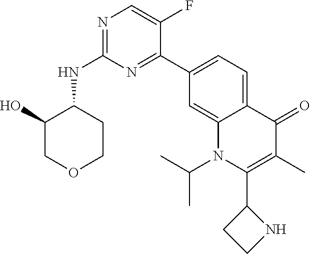 [SMILES: Cc1c(C2CCN2)n(C(C)C)c2cc(-c3nc(N[C@@H]4CCOC[C@H]4O)ncc3F)ccc2c1=O]

To a solution of tert-butyl 2-(7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinolin-2-yl)azetidine-1-carboxylate (150 mg, 264 μmol) in dichloromethane (3 mL) was added zinc bromide (297 mg, 1.32 mmol) and the reaction mixture was stirred at 25° C. for 12 h before concentration. The residue was purification by prep-HPLC (column: Phenomenex Gemini NX—C18, 75×30 mm, 5 um; mobile phase: acetonitrile in water (with 10 mM NH4HCO3), 10%-50% gradient in 8.0 min); detector, UV 254 nm. The title compound (25 mg, 92%) was obtained. ¹H NMR (400 MHz, DMSO-d6) δ=8.65 (br d, J=1.2 Hz, 1H), 8.50 (d, J=3.6 Hz, 1H), 8.26 (d, J=8.4 Hz, 1H), 7.87 (d, J=8.4 Hz, 1H), 7.27 (d, J=7.8 Hz, 1H), 5.42 (t, J=7.8 Hz, 1H), 5.19-5.28 (m, 1H), 4.95 (d, J=5.2 Hz, 1H), 3.84 (d, J=5.2, 10.8 Hz, 4H), 3.49-3.63 (m, 2H), 3.12 (t, J=6.4 Hz, 1H), 3.03 (t, J=10.4 Hz, 1H), 2.70-2.79 (m, 1H), 2.06 (s, 3H), 2.02 (d. J=7.2 Hz, 1H), 1.77 (t, J=6.4 Hz, 3H), 1.66-1.74 (m, 1H), 1.51-1.59 (m, 4H), 1.45-1.51 (m, 1H). LC-MS (M+H)⁺=468.3.

### Example 387: 7-(5-fluoro-2-(((3S,4R)-3-hydroxytetrahydro-2H-pyran-4-yl)amino)pyrimidin-4-yl)-1-isopropyl-3-methyl-2-(1-methylazetidin-2-yl)quinolin-4(1H)-one

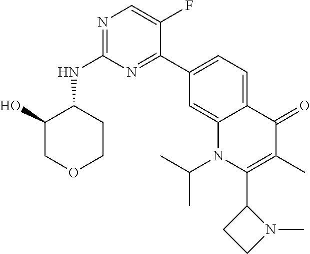 [SMILES: Cc1c(C2CCN2C)n(C(C)C)c2cc(-c3nc(N[C@@H]4CCOC[C@H]4O)ncc3F)ccc2c1=O]

To a solution of 2-(azetidin-2-yl)-7-(5-fluoro-2-(((3S,4R)-3-hydroxytetrahydro-2H-pyran-4-yl)amino)pyrimidin-4-yl)-1-isopropyl-3-methylquinolin-4(1H)-one (30 mg, 64.17 μmol) in methanol (1 mL) were added formaldehyde solution (52.1 mg, 642 μmol, 47.8 μL, 37%) and sodium cyanoborohydride (8.06 mg, 128 μmol). The reaction mixture was stirred at 25° C. for 1 h before quenched by addition of water (3 mL). The mixture was extracted with ethyl acetate (5 mL×3). The combined organic phases were dried over sodium sulfate, filtered and concentrated under reduced pressure. The residue was purification by prep-HPLC (column: Waters Xbridge BEH C18 100×30 mm, 10 um; mobile phase: acetonitrile in water (with 10 mM NH4HCO3); 20%/6-70% gradient in 8.0 min; detector, UV 254 nm. The title compound (11 mg, 35.6%) was obtained. ¹H NMR (400 MHz, DMSO-d6) δ=8.69 (br s, 1H), 8.50 (d, J=3.6 Hz, 1H), 8.29 (d, J=8.4 Hz, 1H), 7.89 (d, J=8.4 Hz, 1H), 7.28 (d, J=8.0 Hz, 1H), 6.36-6.43 (m, 1H), 4.96 (d, J=5.4 Hz, 1H), 4.62 (t, J=8.8 Hz, 1H), 3.83-3.87 (m, 3H), 3.47-3.59 (m, 2H), 3.04 (t, J=10.4 Hz, 1H), 2.94-2.96 (m, 1H), 2.48 (s, 1H), 2.34-2.40 (m, 1H), 2.32 (s, 3H), 2.26 (s, 3H), 2.02 (d, J=12.0 Hz, 1H), 1.69-1.75 (m, 6H), 1.61-1.69 (m, 1H), 1.46-1.58 (m, 1H). LC-MS (M+H)⁺=482.3.

### Example 388: 7-(5-fluoro-2-(((3S,4R)-3-hydroxytetrahydro-2H-pyran-4-yl)amino)pyrimidin-4-yl)-1-isopropyl-2-(pyrrolidin-2-yl)quinolin-4(1H)-one

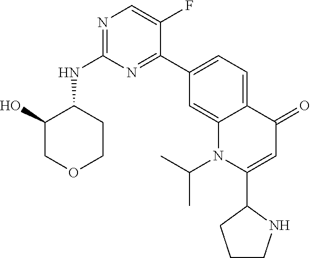 [SMILES: CC(C)n1c(C2CCCN2)cc(=O)c2ccc(-c3nc(N[C@@H]4CCOC[C@H]4O)ncc3F)cc21]

### Step 1: tert-butyl 2-(3-(4-chloro-2-fluorophenyl)-3-oxoprop-1-yn-1-yl)pyrrolidine-1-carboxylate

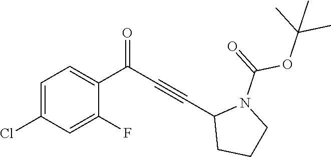 [SMILES: CC(C)(C)OC(=O)N1CCCC1C#CC(=O)c1ccc(Cl)cc1F]

The title compound (1.3 g, 76%) was prepared in a manner similar to that in Example 386 step 1 from 4-chloro-2-fluorobenzoyl chloride and tert-butyl 2-ethynylpyrrolidine-1-carboxylate. LC-MS (M+H)⁺=352.1.

### Step 2: tert-butyl 2-(7-chloro-1-isopropyl-4-oxo-1,4-dihydroquinolin-2-yl)pyrrolidine-1-carboxylate

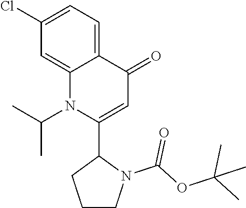 [SMILES: CC(C)n1c(C2CCCN2C(=O)OC(C)(C)C)cc(=O)c2ccc(Cl)cc21]

To a solution of tert-butyl 2-(3-(4-chloro-2-fluorophenyl)-3-oxoprop-1-yn-1-yl)pyrrolidine-1-carboxylate (0.9 g, 2.7 mmol) in dimethylacetamide (10 mL) were added isopropylamine (240 mg, 4.06 mmol) and potassium carbonate (572 mg, 4.14 mmol) in portions at room temperature. The resulting mixture was stirred at 130° C. for 5 h under nitrogen atmosphere before cooled to room temperature. Water (15 mL) was added and the mixture was extracted with ethyl acetate (20 mL×3). The organic phases were combined, washed with brine, dried over sodium sulfate, filtered and concentrated. The residue was purified over silica gel by combi-flash, eluting with ethyl acetate in petroleum ether (0-60% gradient, v/v) to give the title compound (180 mg, 17%). LC-MS (M+H)⁺=391.1.

### Step 3: tert-butyl 2-(1-isopropyl-4-oxo-7-(4,4,5,5-tetramethyl-1,3,2-dioxaborolan-2-yl)-1,4-dihydroquinolin-2-yl)pyrrolidine-1-carboxylate

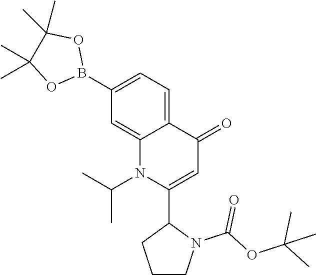 [SMILES: CC(C)n1c(C2CCCN2C(=O)OC(C)(C)C)cc(=O)c2ccc(B3OC(C)(C)C(C)(C)O3)cc21]

The title compound (170 mg, crude for next step) was prepared in a manner similar to that in Example 1 step 6 from tert-butyl 2-(7-chloro-1-isopropyl-4-oxo-1,4-dihydroquinolin-2-yl)pyrrolidine-1-carboxylate. LC-MS (M+H)⁺=483.3.

### Step 4: tert-butyl 2-(7-(2-chloro-5-fluoropyrimidin-4-yl)-1-isopropyl-4-oxo-1,4-dihydroquinolin-2-yl)pyrolidine-1-carboxylate

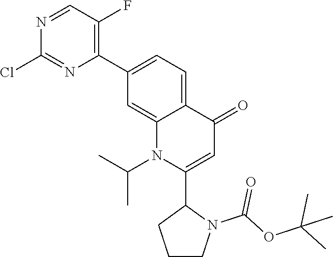 [SMILES: CC(C)n1c(C2CCCN2C(=O)OC(C)(C)C)cc(=O)c2ccc(-c3nc(Cl)ncc3F)cc21]

The title compound (50 mg, 23%) was prepared in a manner similar to that in Example 1 step 7 from tert-butyl 2-(1-isopropyl-4-oxo-7-(4,4,5,5-tetramethyl-1,3,2-dioxaborolan-2-yl)-1,4-dihydroquinolin-2-yl)pyrolidine-1-carboxylate and 2,4-dichloro-5-fluoropyrimidine. LC-MS (M+H)⁺=487.2.

### Step 5: tert-butyl 2-(7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)pyrrolidine-1-carboxylate

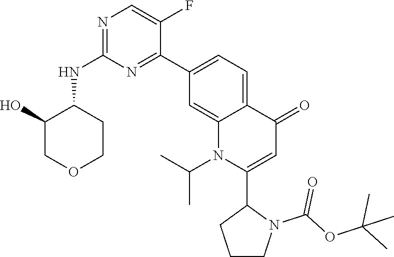 [SMILES: CC(C)n1c(C2CCCN2C(=O)OC(C)(C)C)cc(=O)c2ccc(-c3nc(N[C@@H]4CCOC[C@H]4O)ncc3F)cc21]

The title compound (31 mg, 68%) was prepared in a manner similar to that in Example 1 step 8 from tert-butyl 2-(7-(2-chloro-5-fluoropyrimidin-4-yl)-1-isopropyl-4-oxo-1,4-dihydroquinolin-2-yl)pyrrolidine-1-carboxylate and (3S,4R)-4-aminooxan-3-ol hydrochloride. LC-MS (M+H)⁺=568.3.

### Step 6: 7-(5-fluoro-2-(((3S,4R)-3-hydroxytetrahydro-2H-pyran-4-yl)amino)pyrimidin-4-yl)-1-isopropyl-2-(pyrrolidin-2-yl)quinolin-4(1H)-one

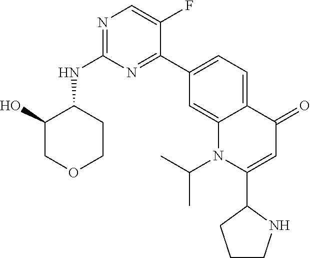 [SMILES: CC(C)n1c(C2CCCN2)cc(=O)c2ccc(-c3nc(N[C@@H]4CCOC[C@H]4O)ncc3F)cc21]

The title compound (7 mg, 27%) was prepared in a manner similar to that in Example 386 step 9 from tert-butyl 2-(7-(5-fluoro-2-(((3S,4R)-3-hydroxytetrahydro-2H-pyran-4-yl)amino)pyrimidin-4-yl)-1-isopropyl-4-oxo-1,4-dihydroquinolin-2-yl)pyrrolidine-1-carboxylate. ¹H NMR (400 MHz, DMSO-d6) δ 8.70 (s, 1H), 8.51 (d, J=3.8 Hz, 1H), 8.29 (d, J=8.4 Hz, 1H), 7.92 (d, J=8.3 Hz. 1H), 7.30 (d, J=8.0 Hz, 1H), 6.65 (s, 1H), 5.32-5.20 (m, 1H), 4.97 (d, J=5.2 Hz, 1H), 4.45-4.37 (m, 1H), 3.90-3.81 (m, 3H), 3.60-3.48 (m, 1H), 3.37-3.32 (m, 1H), 3.09-2.95 (m, 3H), 2.45-2.30 (m, 2H), 2.05-1.98 (m, 1H), 1.83-1.69 (m, 8H), 1.68-1.46 (m, 2H). LC-MS (M+H)⁺=468.1.

### Example 389: 7-(5-fluoro-2-(((3S,4R)-3-hydroxytetrahydro-2H-pyran-4-yl)amino)pyrimidin-4-yl)-1-isopropyl-3-methyl-2-(pyrrolidin-2-yl)quinolin-4(1H)-one

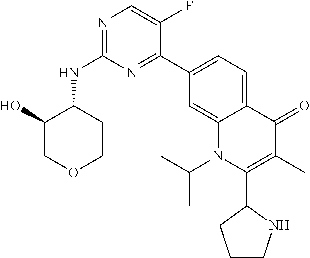 [SMILES: Cc1c(C2CCCN2)n(C(C)C)c2cc(-c3nc(N[C@@H]4CCOC[C@H]4O)ncc3F)ccc2c1=O]

### Step 1: tert-butyl 2-(7-chloro-3-iodo-1-isopropyl-4-oxo-1,4-dihydroquinolin-2-yl)pyrrolidine-1-carboxylate

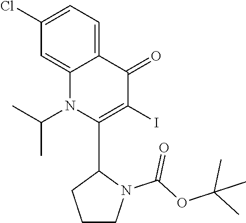 [SMILES: CC(C)n1c(C2CCCN2C(=O)OC(C)(C)C)c(I)c(=O)c2ccc(Cl)cc21]

The title compound (43 mg, 23%) was prepared in a manner similar to that in Example 16 step 1 from tert-butyl 2-(7-chloro-1-isopropyl-4-oxo-1,4-dihydroquinolin-2-yl)pyrrolidine-1-carboxylate. LC-MS (M+H)⁺=517.1.

### Step 2: tert-butyl 2-(7-chloro-1-isopropyl-3-methyl-4-oxo-1,4-dihydroquinolin-2-yl)pyrrolidine-1-carboxylate

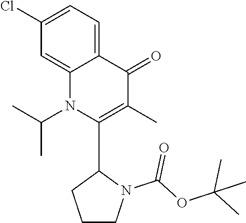 [SMILES: Cc1c(C2CCCN2C(=O)OC(C)(C)C)n(C(C)C)c2cc(Cl)ccc2c1=O]

The title compound (32 mg, 94%) was prepared in a manner similar to that in Example 9 step 2 from tert-butyl 2-(7-chloro-3-iodo-1-isopropyl-4-oxo-1,4-dihydroquinolin-2-yl)pyrrolidine-1-carboxylate. LC-MS (M+H)⁺=405.1.

### Step 3: tert-butyl 2-(1-isopropyl-3-methyl-4-oxo-7-(4,4,5,5-tetramethyl-1,3,2-dioxaborolan-2-yl)-1,4-dihydroquinolin-2-yl)pyrrolidine-1-carboxylate

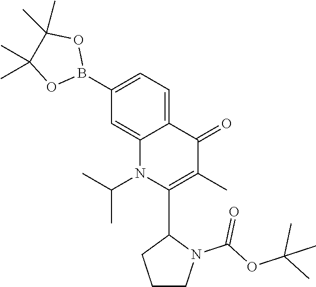 [SMILES: Cc1c(C2CCCN2C(=O)OC(C)(C)C)n(C(C)C)c2cc(B3OC(C)(C)C(C)(C)O3)ccc2c1=O]

The title compound (50 mg, crude used for next step) was prepared in a manner similar to that in Example 1 step 6 from tert-butyl 2-(7-chloro-1-isopropyl-3-methyl-4-oxo-1,4-dihydroquinolin-2-yl)pyrrolidine-1-carboxylate. LC-MS (M+H)⁺=497.4.

### Step 4: tert-butyl 2-(7-(2-chloro-5-fluoropyrimidin-4-yl-1-isopropyl-3-methyl-4-oxo-1,4-dihydroquinolin-2-yl)pyrrolidine-1-carboxylate

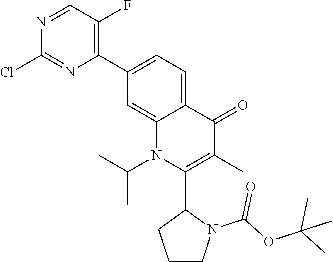 [SMILES: Cc1c(C2CCCN2C(=O)OC(C)(C)C)n(C(C)C)c2cc(-c3nc(Cl)ncc3F)ccc2c1=O]

The title compound (14 mg, 35%) was prepared in a manner similar to that in Example 1 step 7 from tert-butyl 2-(1-isopropyl-3-methyl-4-oxo-7-(4,4,5,5-tetramethyl-1,3,2-dioxaborolan-2-yl)-1,4-dihydroquinolin-2-yl)pyrrolidine-1-carboxylate and 2,4-dichloro-5-fluoropyrimidine. LC-MS (M+H)⁺=501.2.

### Step 5: tert-butyl 2-(7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinolin-2-yl)pyrrolidine-1-carboxylate

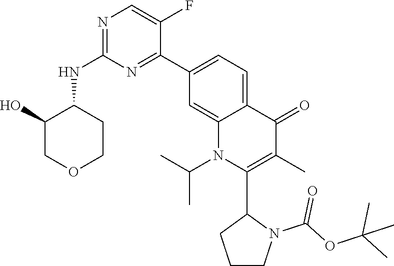 [SMILES: Cc1c(C2CCCN2C(=O)OC(C)(C)C)n(C(C)C)c2cc(-c3nc(N[C@@H]4CCOC[C@H]4O)ncc3F)ccc2c1=O]

The title compound (10 mg, 57%) was prepared in a manner similar to that in Example 1 step 8 from tert-butyl 2-(7-(2-chloro-5-fluoropyrimidin-4-yl)-1-isopropyl-3-methyl-4-oxo-1,4-dihydroquinolin-2-yl)pyrrolidine-1-carboxylate and (3S,4R)-4-aminooxan-3-ol hydrochloride. LC-MS (M+H)⁺=582.4.

### Step 6: 7-(5-fluoro-2-(((3S,4R)-3-hydroxytetrahydro-2H-pyran-4-yl)amino)pyrimidin-4-yl)-1-isopropyl-3-methyl-2-(pyrrolidin-2-yl)quinolin-4(1H)-one

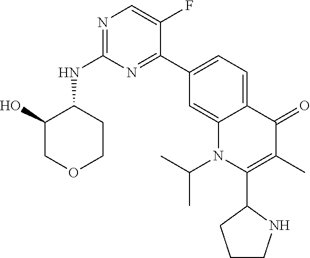 [SMILES: Cc1c(C2CCCN2)n(C(C)C)c2cc(-c3nc(N[C@@H]4CCOC[C@H]4O)ncc3F)ccc2c1=O]

The title compound (1 mg, 11%) was prepared in a manner similar to that in Example 386 step 9 from tert-butyl 2-(7-(5-fluoro-2-(((3S,4R)-3-hydroxytetrahydro-2H-pyran-4-yl)amino)pyrimidin-4-yl)-1-isopropyl-3-methyl-4-oxo-1,4-dihydroquinolin-2-yl)pyrrolidine-1-carboxylate. ¹H NMR (400 MHz, DMSO-d6) δ 8.73-8.68 (m, 1H), 8.50 (d, J=3.8 Hz, 1H), 8.34-8.27 (m, 1H), 7.89 (d, J=8.3 Hz, 1H), 7.28 (d, J=7.9 Hz, 1H), 5.71-5.59 (m, 1H), 4.96 (d, J=5.2 Hz, 1H), 4.79-4.70 (m, 1H), 3.89-3.81 (m, 3H), 3.60-3.49 (m, 1H), 3.33-3.28 (m, 2H), 3.09-2.92 (m, 3H), 2.57-2.52 (m, 1H), 2.36-2.25 (m, 2H), 2.20 (s, 3H), 2.00 (s, 1H), 1.78-1.70 (m, 3H), 1.69-1.61 (m, 3H), 1.58-1.44 (m, 1H). LC-MS (M+H)⁺=482.1.

### Biological Assays

Compounds disclosed herein were tested for inhibition of CDK4/Cyclin D1 or CDK6/Cyclin D3 kinase in an assay based on the time-resolved fluorescence-resonance energy transfer (TR-FRET) methodology. The assay was carried out in 384-well low volume black plates in a reaction mixture containing CDK4/Cyclin D1 or CDK6/Cyclin D3, 1 mM ATP, 0.15 μM Rb (Ser780)-biotin substrate and 0-10 μM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK4/Cyclin D1 or CDK6/Cyclin D3 kinase activity in presence of increasing concentrations of compounds was calculated based on the ratio of fluorescence at 665 nm to that at 620 nm. The IC50 for each compound was derived from fitting the data to the four-parameter logistic equation by Dotmatics.

TABLE 1
Enzymatic activity IC50 (nM) for the compounds disclosed herein

| Example No. | CDK4D1 IC50 (nM) | CDK6D3 IC50 (nM) |
|---|---|---|
| 1 | 101 | 808 |
| 2 | 68 | 1224 |
| 3 | 78 | 1248 |
| 4 | 251 | 978.9 |
| 5 | 249 | 3237 |
| 6 | 15 | 345 |
| 7 | 14 | 574 |
| 8 | 162 | 1782 |
| 9 | 59 | 590 |
| 10 | 40 | 1440 |
| 11 | 28 | 420 |
| 12 | 258 | 1935 |
| 13 | 93 | 539.4 |
| 14 | 42 | 714 |
| 15 | 15 | 255 |
| 16 | 8.3 | 257.3 |
| 17 | 7.5 | 120 |
| 18 | 27 | 1215 |
| 19 | 24 | 504 |
| 20 | 15 | 510 |
| 21 | 35 | 385 |
| 22 | 49 | 931 |
| 23 | 53 | 445.2 |
| 24 | 24 | 432 |
| 25 | 8.2 | 139.4 |
| 26 | 16 | 336 |
| 27 | 5.7 | 148.2 |
| 28 | 17 | 374 |
| 29 | 19 | 361 |
| 30 | 30 | 780 |
| 31 | 13 | 481 |
| 32 | 10 | 200 |
| 33 | 12 | 324 |
| 34 | 10 | 430 |
| 35 | 6.9 | 276 |
| 36 | 57 | 552.9 |
| 37 | 22 | 682 |
| 38 | 24 | 888 |
| 39 | 24 | 744 |
| 40 | 8.8 | 149.6 |
| 41 | 24 | 1296 |
| 42 | 15 | 645 |
| 43 | 25 | 1300 |
| 44 | 29 | 1276 |
| 45 | 21 | 651 |
| 46 | 12 | 480 |
| 47 | 14 | 658 |
| 48 | 9.1 | 209.3 |
| 49 | 8.3 | 298.8 |
| 50 | 16 | 688 |
| 51 | 54 | 648 |
| 52 | 6.4 | 467.2 |
| 53 | 11 | 165 |
| 54 | 13 | 260 |
| 55 | 15 | 345 |
| 56 | 7 | 238 |
| 57 | 16 | 352 |
| 58 | 3.4 | 78.2 |
| 59 | 27 | 675 |
| 60 | 22 | 638 |
| 61 | 30 | 1170 |
| 62 | 17 | 255 |
| 63 | 22 | 484 |
| 64 | 26 | 702 |
| 65 | 31 | 744 |
| 66 | 21 | 651 |
| 67 | 21 | 483 |
| 68 | 34 | 1224 |
| 69 | 21 | 693 |
| 70 | 11 | 396 |
| 71 | 2.7 | 140.4 |
| 72 | 0.8 | 21.6 |
| 73 | 7.5 | 225 |
| 74 | 30 | 810 |
| 75 | 21 | 525 |
| 76 | 28 | 588 |
| 77 | 26 | 598 |
| 78 | 22 | 352 |
| 79 | 34 | 850 |
| 80 | 22 | 616 |
| 81 | 23 | 483 |
| 82 | 12 | 540 |
| 83 | 32 | 928 |
| 84 | 6.8 | 170 |
| 85 | 19 | 494 |
| 86 | 21 | 777 |
| 87 | 22 | 682 |
| 88 | 4.1 | 405.9 |
| 89 | 3.8 | 406.6 |
| 90 | 5.7 | 250.8 |
| 91 | 24 | 1056 |
| 92 | 4.1 | 131.2 |
| 93 | 8.7 | 295.8 |
| 94 | 11 | 165 |
| 95 | 13 | 208 |
| 96 | 21 | 273 |
| 97 | 64 | 2048 |
| 98 | 8.6 | 369.8 |
| 99 | 17 | 561 |
| 100 | 19 | 760 |
| 101 | 26 | 312 |
| 102 | 15 | 330 |
| 103 | 12 | 516 |
| 104 | 24 | 139.2 |
| 105 | 4.1 | 291.1 |
| 106 | 4.8 | 297.6 |
| 107 | 7.4 | 340.4 |
| 108 | 14 | 392 |
| 109 | 6.2 | 210.8 |
| 110 | 9.3 | 567.3 |
| 111 | 16 | 352 |
| 112 | 15 | 615 |
| 113 | 17 | 663 |
| 114 | 40 | 640 |
| 115 | 6.6 | 270.6 |
| 116 | 3.9 | 175.5 |
| 117 | 1.9 | 96.9 |
| 118 | 1.1 | 51.7 |
| 119 | 9.1 | 273 |
| 120 | 5.9 | 265.5 |
| 121 | 4.7 | 112.8 |
| 122 | 1.6 | 68.8 |
| 123 | 4.3 | 129 |
| 124 | 5 | 230 |
| 125 | 15 | 360 |
| 386 | 12 | 375 |
| 387 | 5.1 | 237 |
| 388 | 10 | 450 |
| 389 | 1.6 | 220 |

What we claimed is:

1. A compound of formula (I): 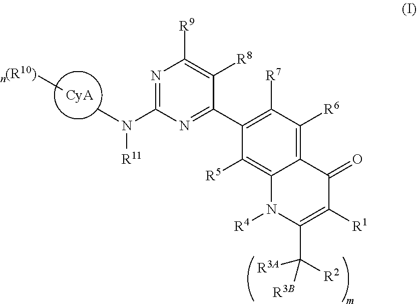
or a N-oxide thereof, or a pharmaceutically acceptable salt thereof, or a stereoisomer thereof, or a tautomer thereof, or a deuterated analog thereof, or a prodrug thereof,
wherein:
ring CyA is a 3- to 8-membered ring, said ring comprising 0, 1 or 2 heteroatom(s) independently selected from nitrogen, oxygen, or optionally oxidized sulfur as ring member(s); said ring is optionally substituted with at least one substituent R¹⁰;
n is 0, 1, 2, 3, 4 or 5;
m is 0 or 1; provided that when m=0, the 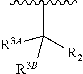
moiety as a whole is replaced with H;
R¹ is H, halogen, —C₁₋₈alkyl, —C₂₋₈alkenyl, —C₂₋₈alkynyl, cycloalkyl, haloalkyl, heterocyclyl, aryl, heteroaryl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f};
R^{1d} and R^{1f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2a}, —SO₂R^{2a}, —SO₂NR^{2a}R^{2b}, —COR^{2a}, —CO₂R^{2a}, —CONR^{1a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2d};
R^{2a}, R^{2b} and R^{2c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(two R^{2g}) and/or (two R^{2h}) together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent selected from the group consisting of halogen, hydroxy, oxo, —C₁₋₈alkyl, -haloC₁₋₈alkyl, —C(O)C₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2g}, R^{2h} and R²ⁱ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, —C(O)C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3A} and R^{3B} are each independently hydrogen, halogen, —C₁₋₈alkyl, —C₂₋₈alkenyl, —C₂₋₈alkynyl, cycloalkyl, heterocyclyl, aryl, heteroaryl or —CN; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3c}; or
R^{3A} and R^{3B} together with the atom to which they are attached, form an oxo group (—C(═O)—) or a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3d}, R^{3e} and R^{3f} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁴ is hydrogen, —C₁₋₈alkyl, —C₂₋₈alkenyl, —C₂₋₈alkynyl, cycloalkyl or heterocyclyl; wherein each of said —C₁₋₈alkyl, —C₂₋₈alkenyl, —C₂₋₈alkynyl, cycloalkyl or heterocyclyl is optionally substituted with at least one substituent R^{4a};
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵, R⁶, R⁷, R⁸ and R⁹ are each independently selected from H, halogen, —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5f};
R^{5d} and R^{5f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹⁰ is selected from H, halogen, —C₁₋₈alkyl, —C₂₋₈alkenyl, —C₂₋₈alkynyl, cycloalkyl, heterocyclyl, aryl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10d};
R^{10a}, R^{10b} and R^{10c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10f};
R^{10d} and R^{10f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹¹ is selected from H, —C₁₋₈alkyl, —C₂₋₈alkenyl, —C₂₋₈alkynyl; wherein each of said —C₁₋₈alkyl, —C₂₋₈alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

2. The compound of claim 1, wherein the compound is selected from formula (IIa), (IIb), (IIc), (IId) or (IIe): 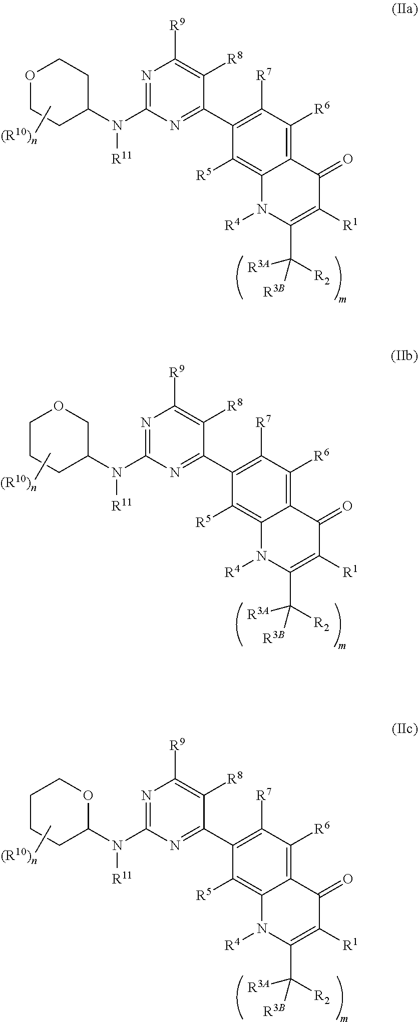 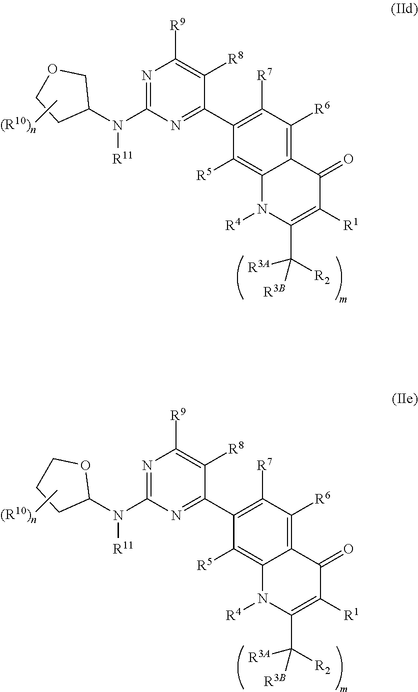
wherein, R¹, R², R^{3A}, R^{3B}, R⁴, R⁵, R⁶, R⁷, R⁸, R⁹, R¹⁰, R¹¹, m and n are each defined as claim 1;
preferably, the compound is selected from formula (IIf), (IIg), (IIh) or (IIi): 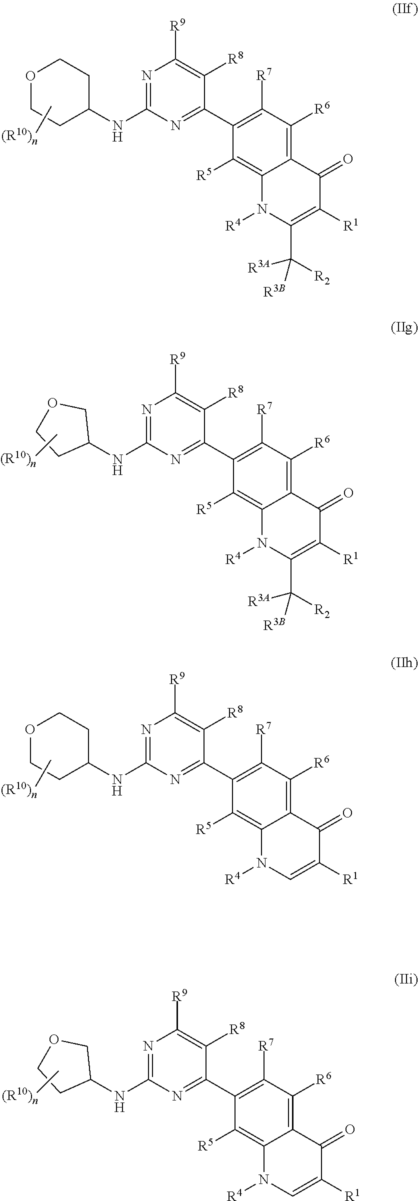
wherein, R¹, R², R^{3A}, R^{3B}, R⁴, R⁵, R⁶, R⁷, R⁸, R⁹, R¹⁰ and n are each defined as claim 1;
more preferably, the compound is selected from formula (IIj), (IIk), (IIl) or (IIm): 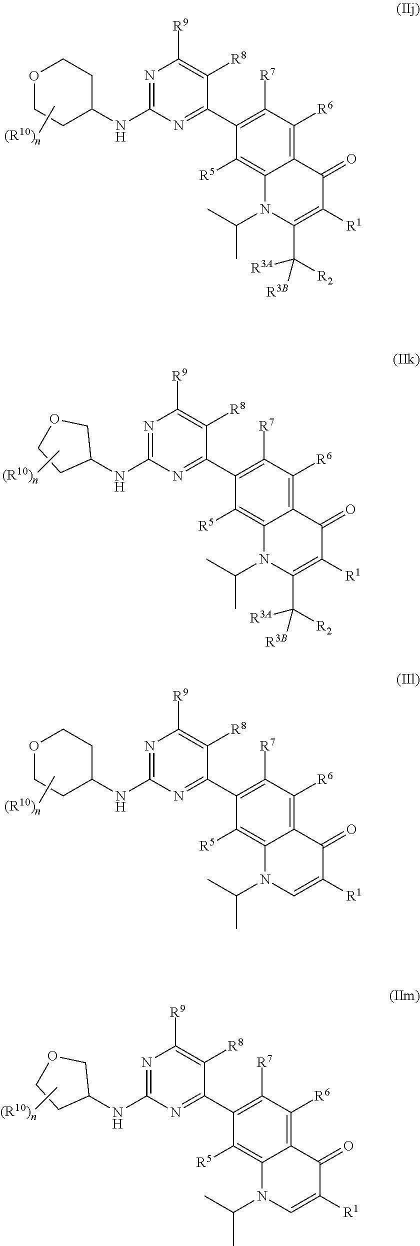
wherein, R¹, R², R^{3A}, R^{3B}, R⁵, R⁶, R⁷, R⁸, R⁹, R¹⁰ and n are each defined as claim 1;
even more preferably, the compound is selected from formula (IIn), (IIo), (IIp) or (IIq): 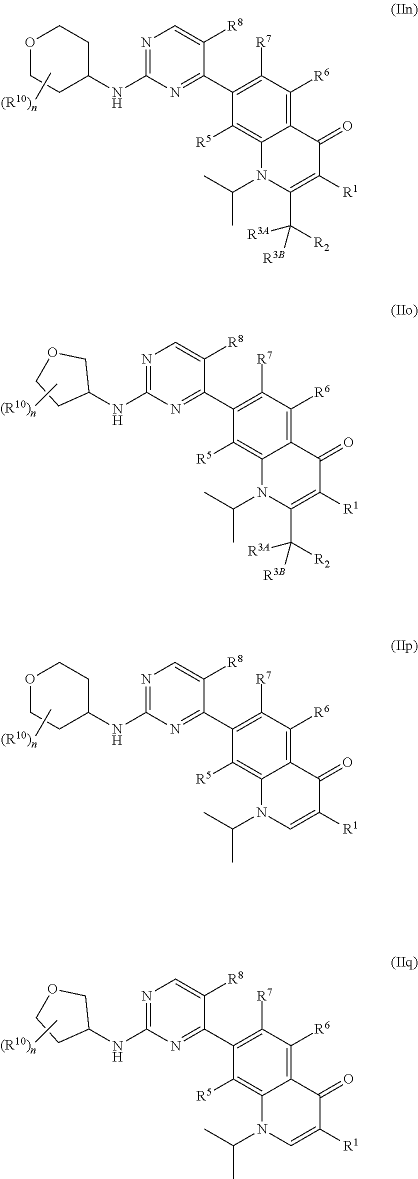
wherein, R¹, R², R^{3A}, R^{3B}, R⁵, R⁶, R⁷, R⁸, R¹⁰, m and n are each defined as claim 1.

3. The compound of anyone of the preceding claims, wherein ring CyA is a 3-, 4-, 5-, 6-, 7- or 8-membered ring, said ring comprising 0, 1 or 2 heteroatom(s) independently selected from nitrogen, oxygen, or optionally oxidized sulfur as ring member(s); said ring is optionally substituted with 0, 1, 2, 3, 4 or 5 R¹⁰; said ring is a saturated or unsaturated ring;
preferably CyA is a 3-, 4-, 5-, 6-, 7- or 8-membered saturated ring, said ring comprising 0, 1 or 2 heteroatom(s) independently selected from nitrogen, oxygen, or optionally oxidized sulfur as ring member(s); said ring is optionally substituted with 0, 1, 2 or 3 R¹⁰;
more preferably CyA is a 5-, 6- or 7-membered saturated ring, said ring comprising 1 or 2 heteroatom(s) independently selected from nitrogen or oxygen as ring member(s); said ring is optionally substituted with 0, 1, 2 or 3 R¹⁰;
even more preferably, CyA is a ring selected from tetrahydrofuranyl or tetrahydropyranyl; said ring is optionally substituted with 0, 1, 2 or 3 R¹⁰.

4. The compound of anyone of the preceding claims, wherein ring CyA is 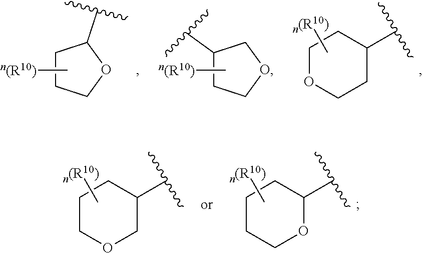
preferably, CyA is 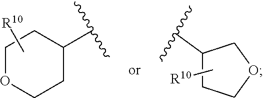
more preferably, CyA is 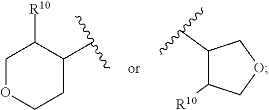
even more preferably, CyA is

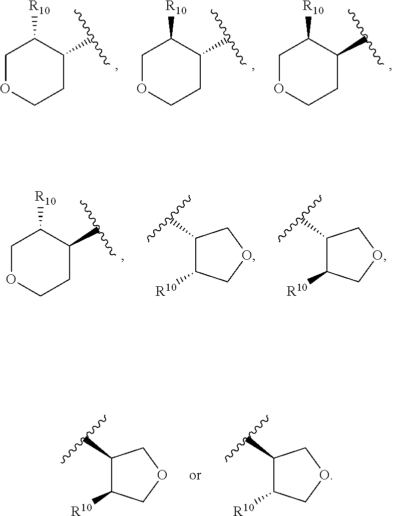

5. The compound of anyone of the preceding claims, wherein R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10d};
R^{10a}, R^{10b} and R^{10c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10f};
R^{10d} and R^{10f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OH or —NH₂;
more preferably, R¹⁰ is —OH.

6. The compound of anyone of the preceding claims, wherein the 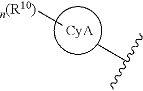
moiety is

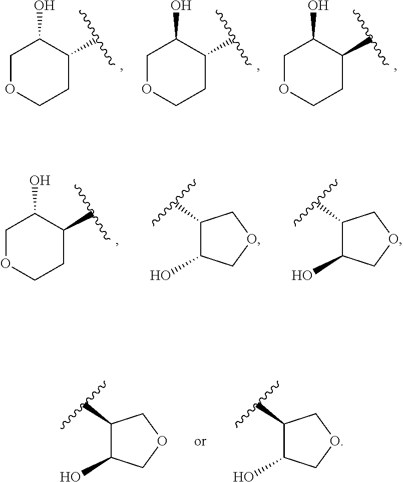

7. The compound of anyone of the preceding claims, wherein R¹ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl or heterocyclyl is optionally substituted with at least one substituent R^{1d};
R^{1a}, R^{1b} and R^{1c} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{1f};
R^{1d} and R^{1f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹ is H, —F, —Cl, —Br, —I, methyl, ethyl, propyl, butyl, pentyl, hexyl, heptyl, octyl, —C₂₋₈alkynyl, cyclopropyl, cyclobutyl, cyclopentyl, cyclohexyl, cycloheptyl, cyclooctyl, —OR^{1a}, —COR^{1a}; wherein each of said methyl, ethyl, propyl, butyl, pentyl, hexyl, heptyl, octyl, —C₂₋₈alkynyl, cyclopropyl, cyclobutyl, cyclopentyl, cyclohexyl, cycloheptyl or cyclooctyl is optionally substituted with at least one substituent selected from hydrogen, —F, —Cl, —Br, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{1a} is selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl;
more preferably, R¹ is H, —F, —Cl, —Br, —I, C₁₋₈alkoxy-C₁₋₈alkyl-, methoxy, ethoxy, propoxy, butoxy, pentoxy, hexoxy, hepthoxy, octoxy, methyl, ethyl, propyl, butyl, pentyl, hexyl, heptyl, octyl, cyclopropyl, cyclobutyl, cyclopentyl, cyclohexyl, cycloheptyl, cyclooctyl, haloalkyl, heterocyclyl or —C(O)C₁₋₈alkyl;
even more preferably, R¹ is H, methyl, ethyl, n-propyl, iso-propyl, n-butyl, sec-butyl, iso-butyl, tert-butyl, —C(O)CH₃, —C(CH₃)₂OH, —OMe, —F, —Cl, cyclopropyl or cyclobutyl.

8. The compound of anyone of the preceding claims, wherein R² is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2d};
R^{2a}, R^{2b} and R^{2c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) and/or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

9. The compound of anyone of the preceding claims, wherein R² is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2a}, —COR^{2a}, —CO₂R^{2a}, —CONR^{2a}R^{2b}, —NR^{2a}R^{2b}, —NR^{2a}COR^{2b}, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{1f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2g}, —SO₂R^{2g}, —COR^{2g}, —CO₂R^{2g}, —CONR^{2g}R^{2h}, —NO₂, —NR^{2g}R^{2h} or —NR^{2g}COR^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) and/or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

10. The compound of anyone of the preceding claims, wherein R² is hydrogen, methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, —OR^{2a}, —NR^{2a}R^{2b} or —NR^{2a}COR^{2b}; wherein each of said methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl (n-propyl or iso-propyl), butyl (n-butyl, sec-butyl, iso-butyl or tert-butyl), pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, —CF₃, —CF₂H, —CFH₂, —CH₂CF₃, —CF₂CH₃, —CH₂OH, —CH(CH₃)OH, —C(CH₃)₂OH, —CH₂CH₂OH, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OH, methoxy, ethoxy, propoxy, butoxy, pentoxy, hexoxy, hepthoxy, octoxy, —SO₂Me, —SO₂Et, —SO₂C₃H₇, —COMe, —COEt, —COC₃H₇, —NH₂, —NHCH₃, —N(CH₃)₂, —NHC₂H₅, —NHC₃H₇, —NHC₄H₉, —CONH₂, —CONHCH₃, —CON(CH₃)₂, —CONHC₂H₅, —CONHC₃H₇, —CONHC₄H₉.

11. The compound of anyone of the preceding claims, wherein R² is —H, -Me, —OMe, —OH, —NH₂, —NHCH₃, —N(CH₃)₂, —NHCH(CH₃)₂, —NHC(CH₃)₃, —NHCOCH₃,

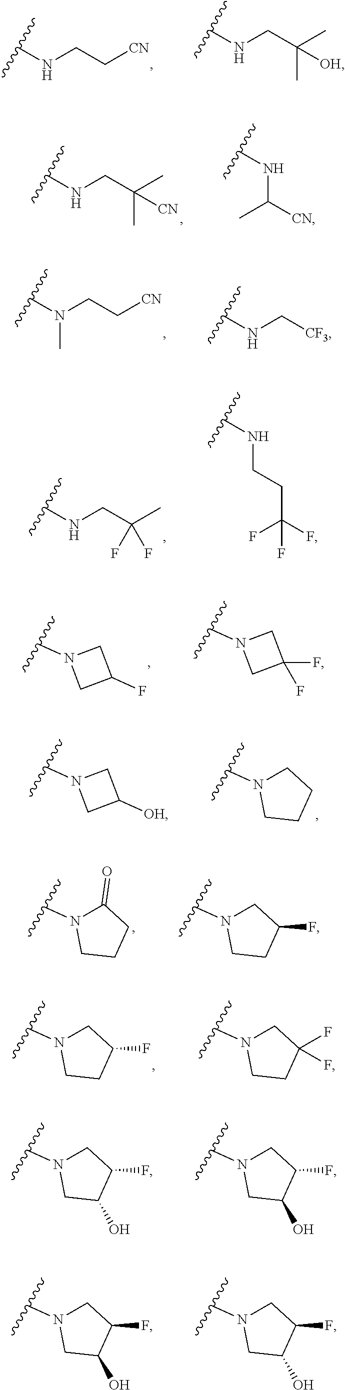

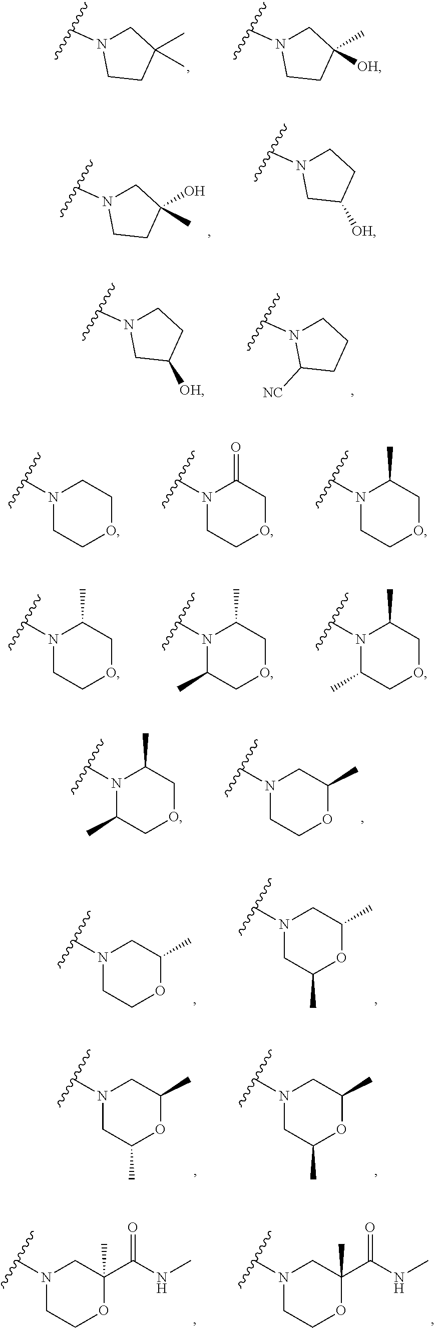

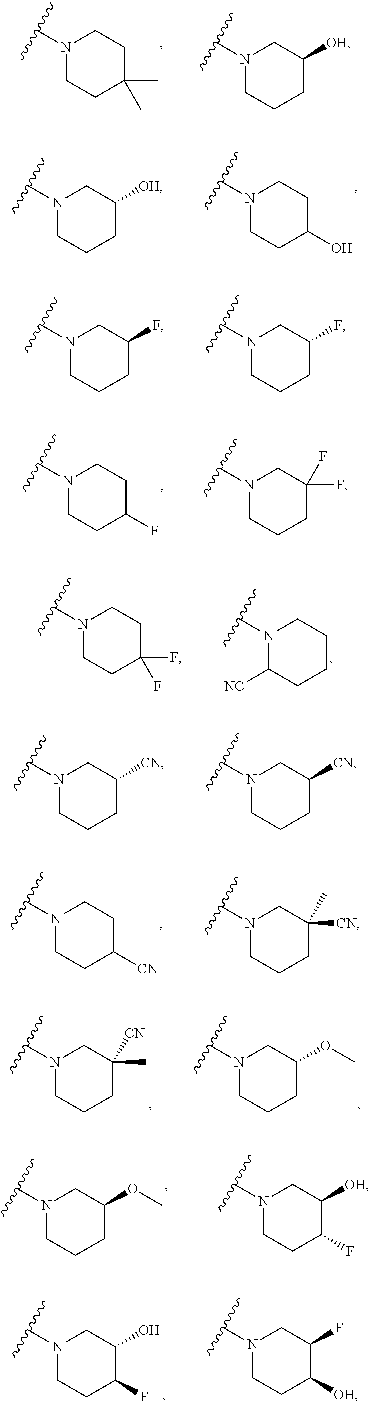

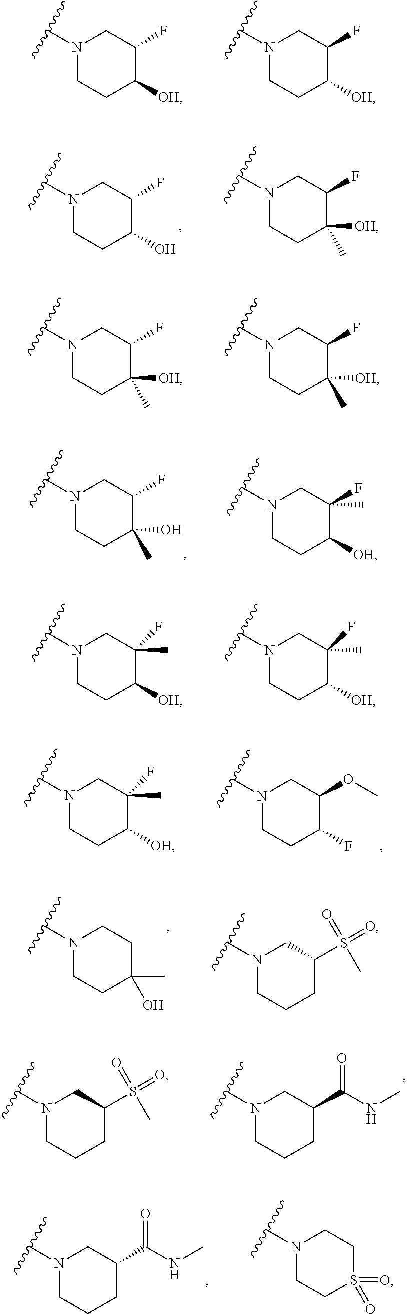

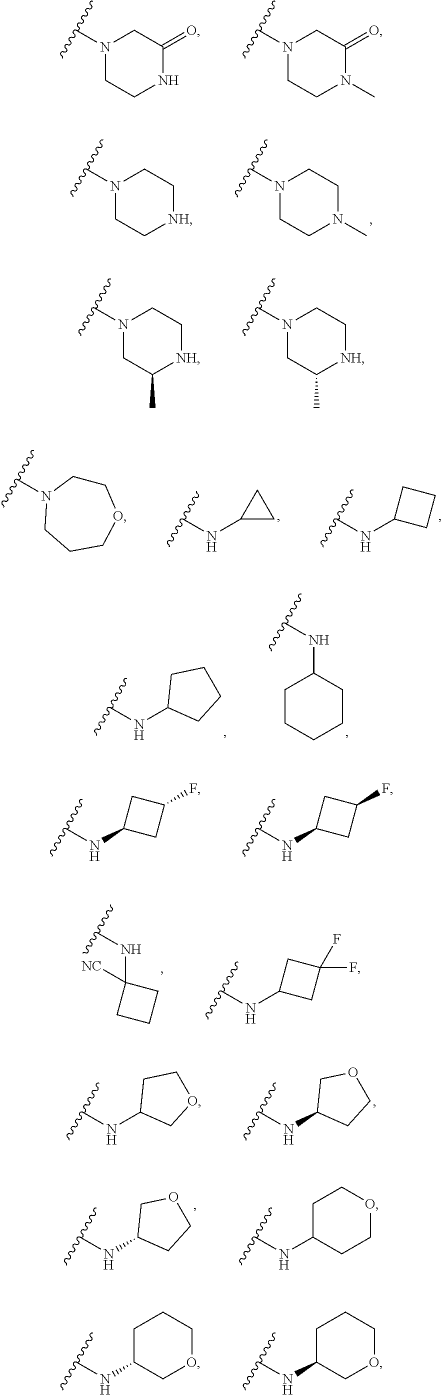

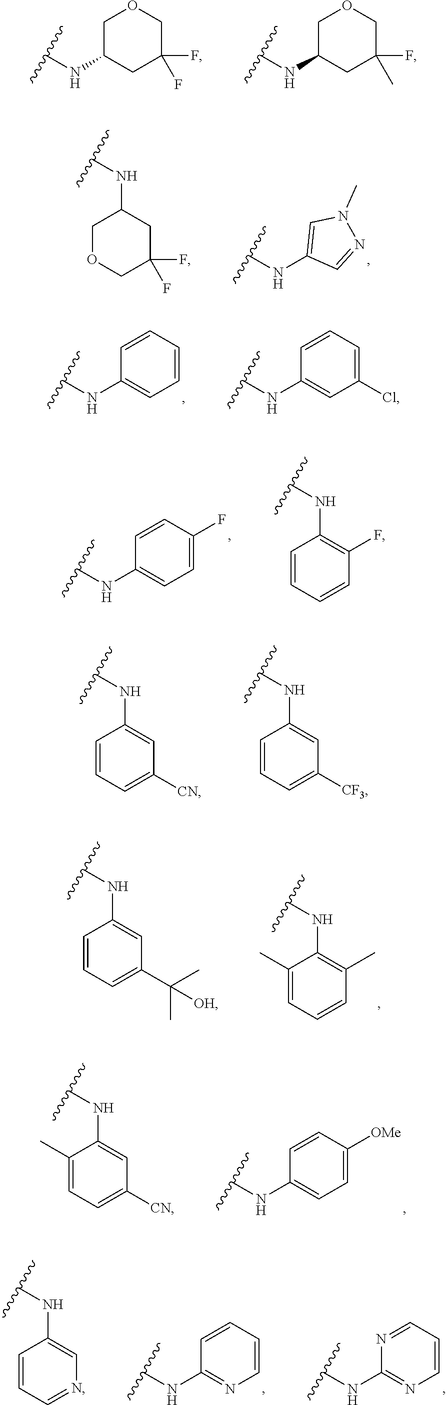

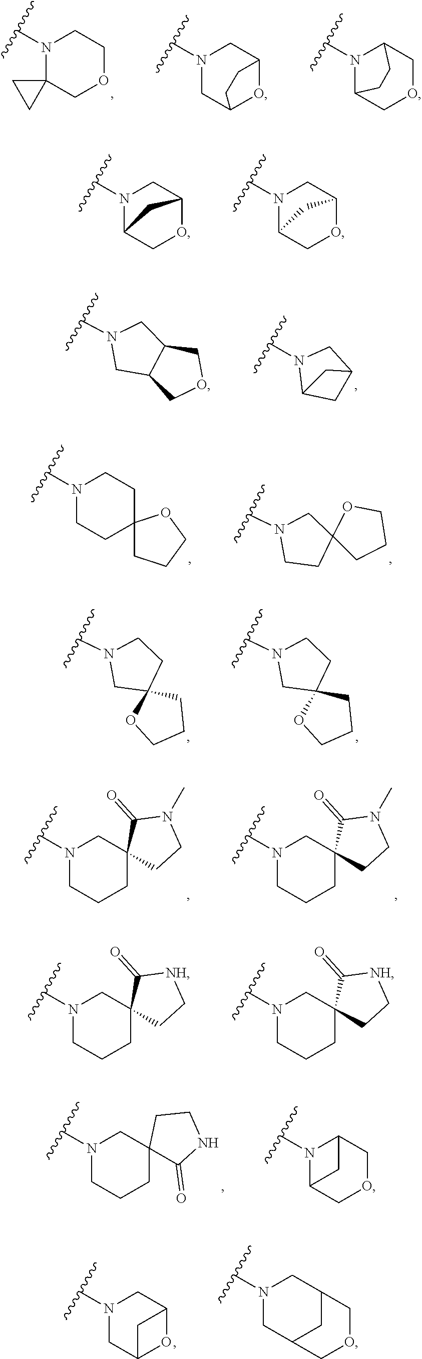

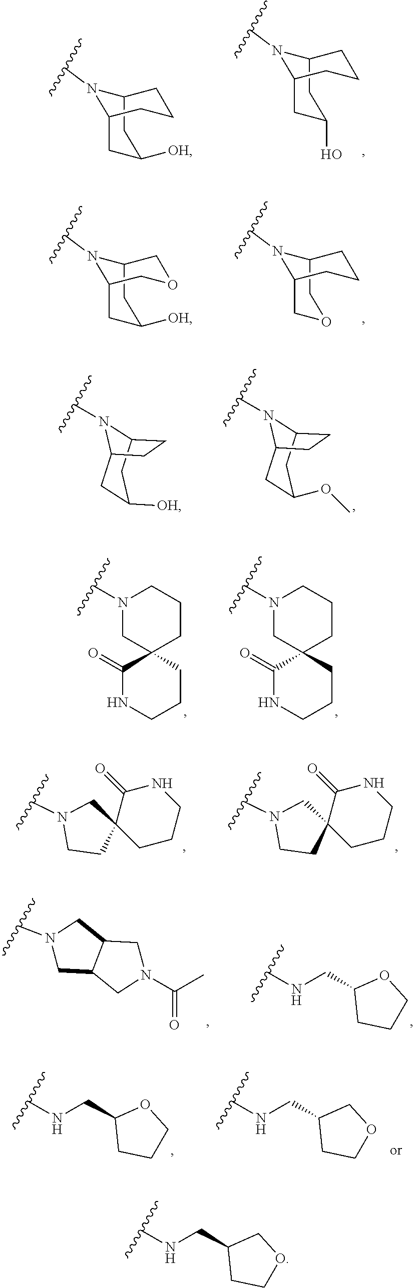

12. The compound of anyone of the preceding claims, wherein R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3c}; or
R^{3A} and R^{3B} together with the atom to which they are attached, form an oxo group (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d}, R^{3e} and R^{3f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
preferably, R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; or
R^{3A} and R^{3B} together with the atom to which they are attached, form an oxo group (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s);
preferably, R^{3A} and R^{3B} together with the atom to which they are attached, form an oxo group (—C(═O)—);
more preferably, R^{3A} and R^{3B} are each independently hydrogen, methyl, ethyl, propyl, butyl or pentyl; or
R^{3A} and R^{3B} together with the atom to which they are attached, form an oxo group (—C(═O)—) or a 3-, 4-, 5-, 6-, 7- or 8-membered ring, said ring comprising 0, 1 or 2 heteroatom(s) independently selected from nitrogen or oxygen.

13. The compound of anyone of the preceding claims, wherein the 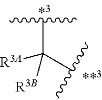
moiety is 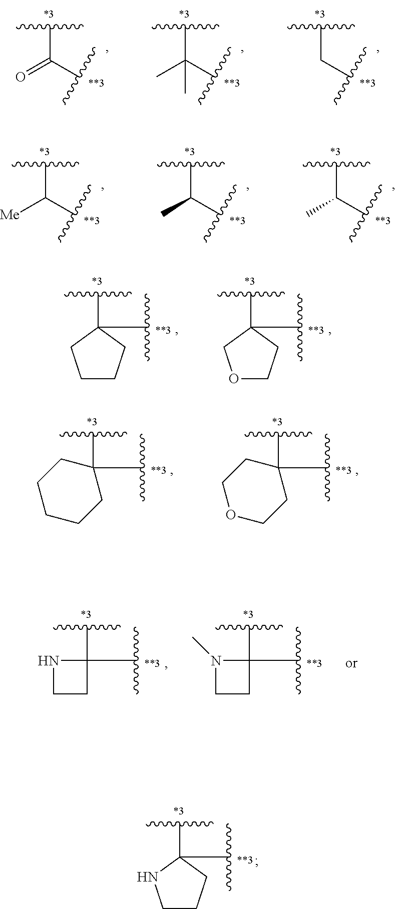
wherein *³ refers to the position attached 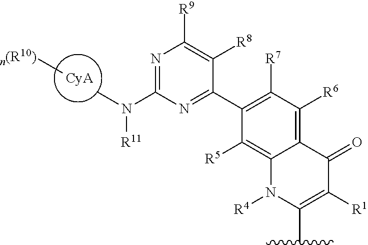
moiety, and **³ refers to the position attached to the 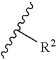
moiety.

14. The compound of anyone of the preceding claims, wherein the 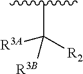
moiety is -Me, -Et,

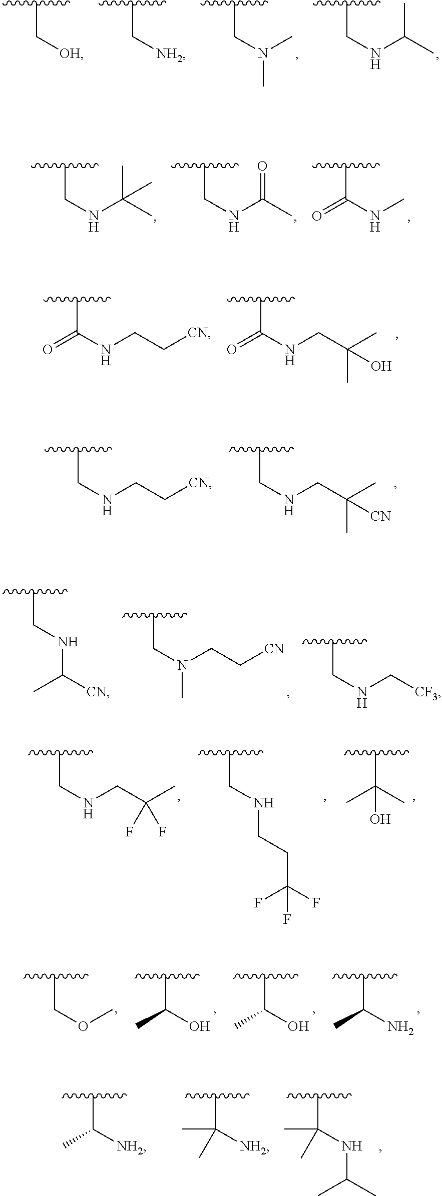

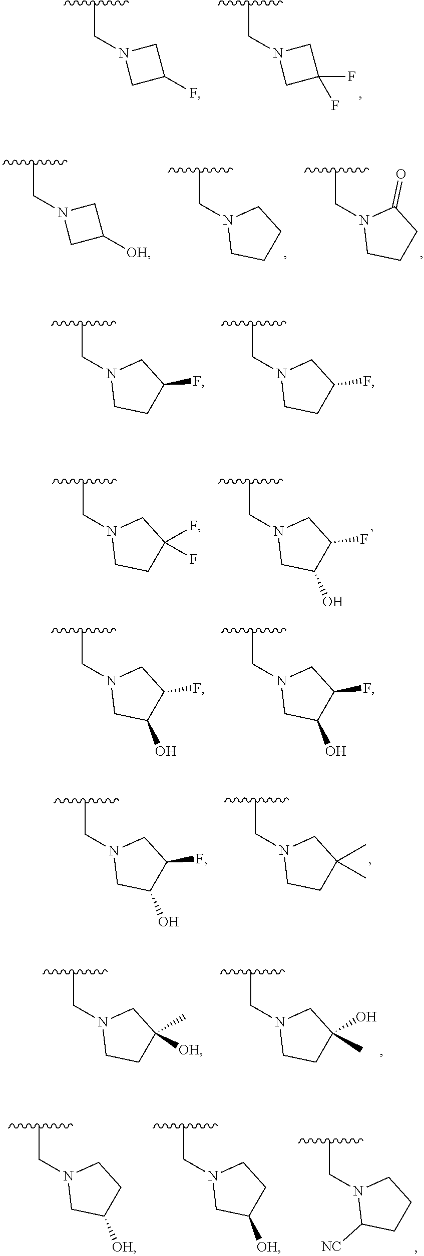

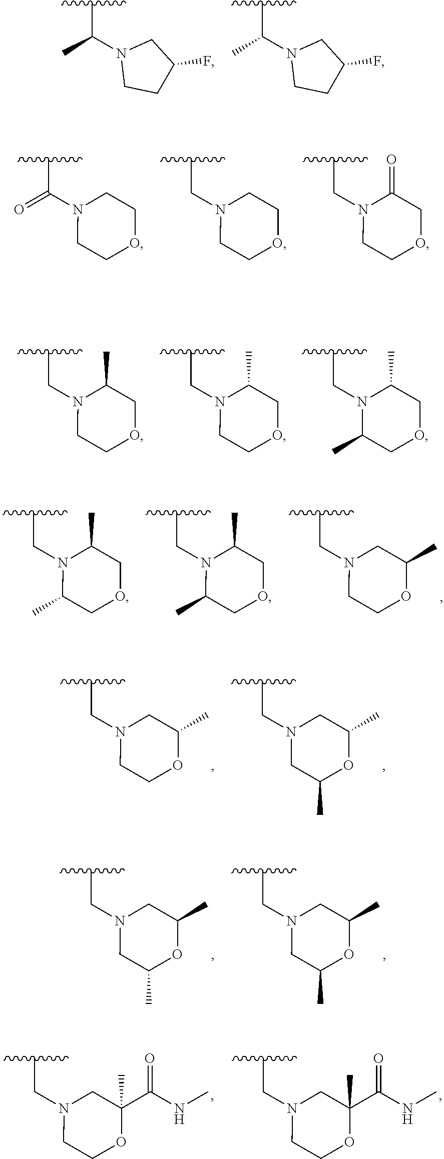

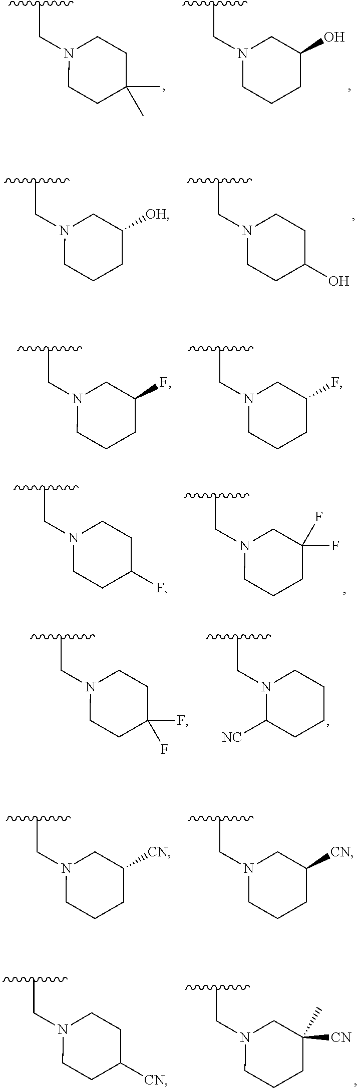

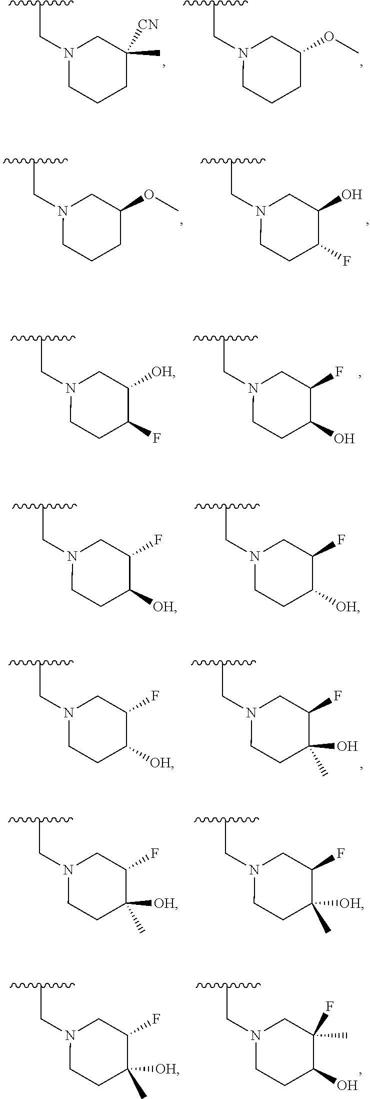

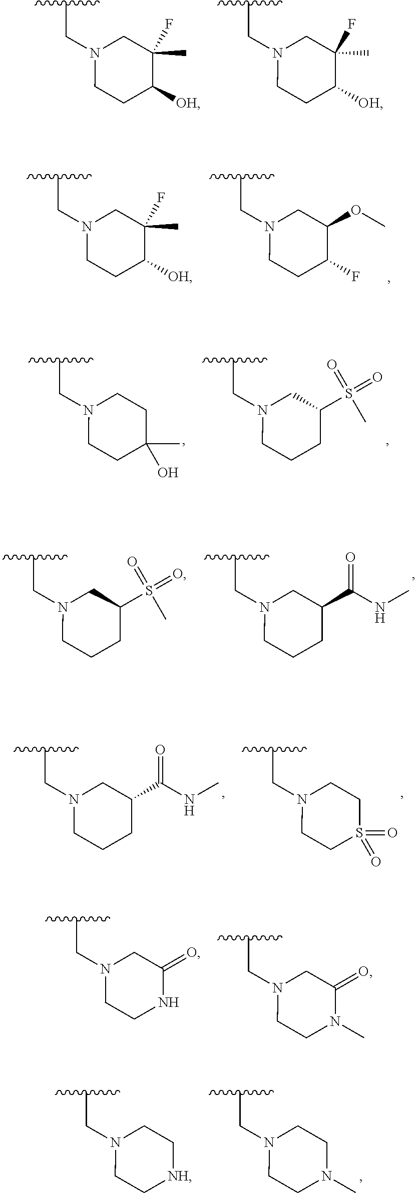

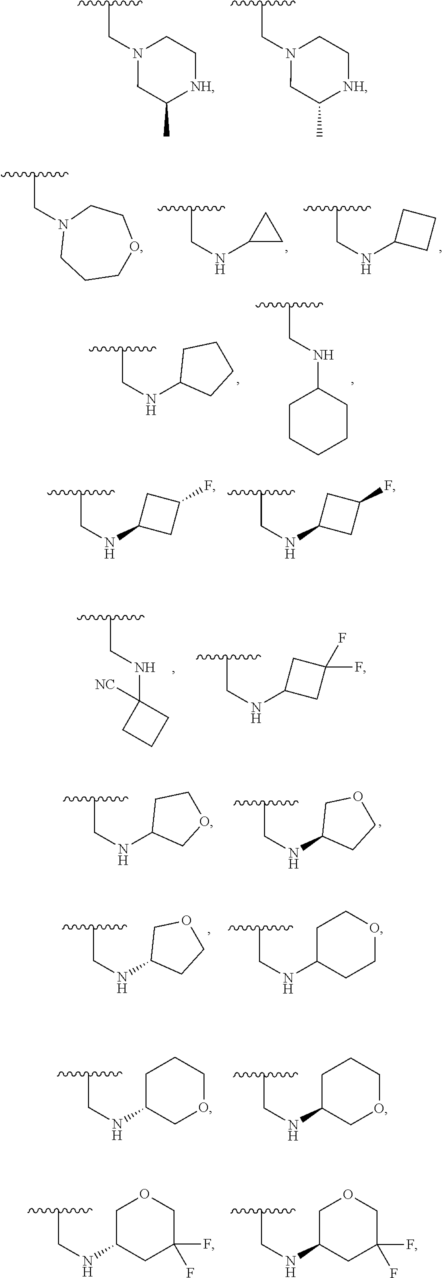

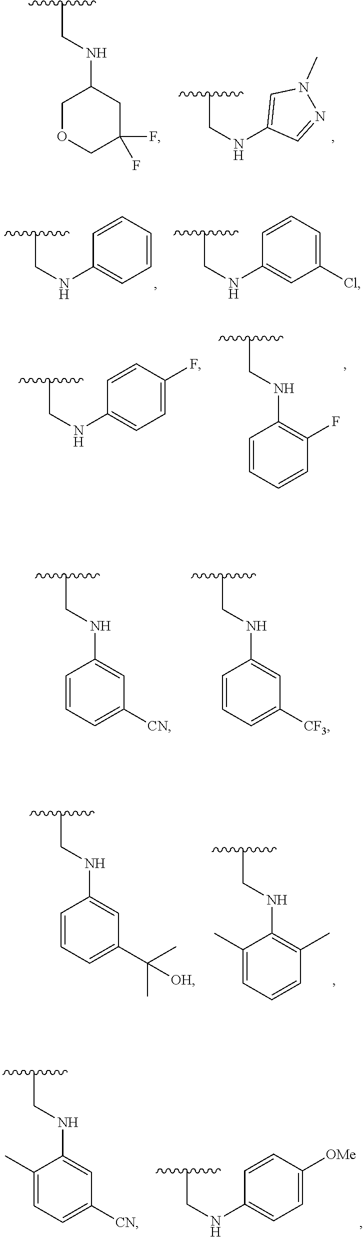

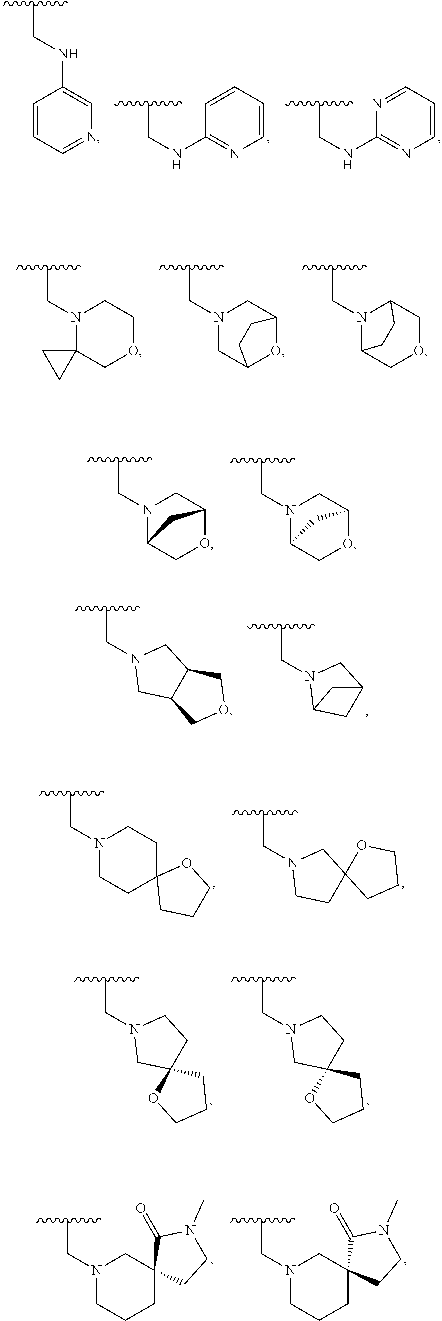

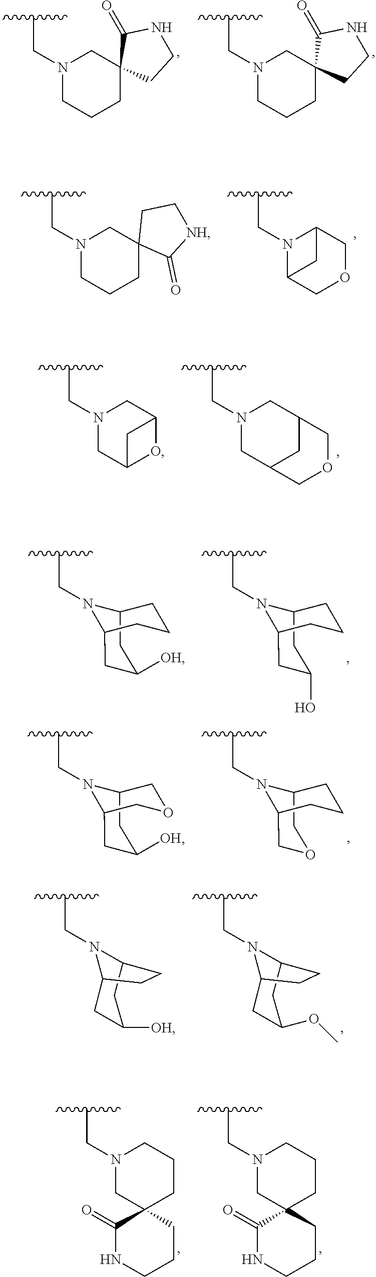

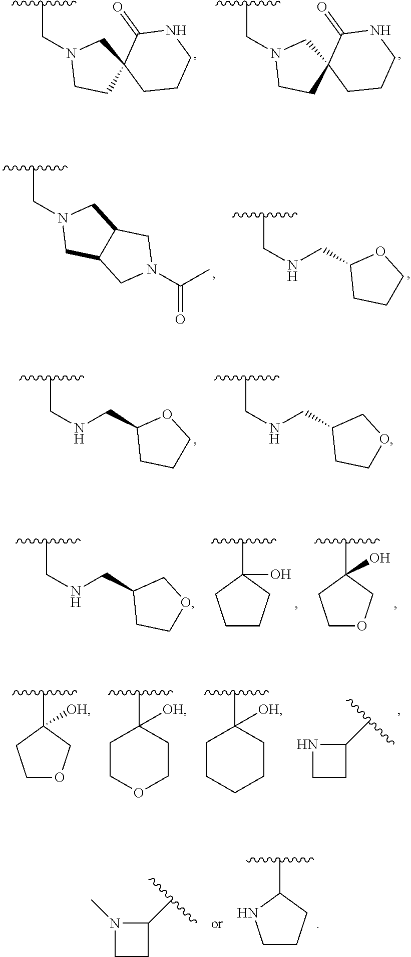

15. The compound of anyone of the preceding claims, wherein W is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl is optionally substituted with at least one substituent R^{4a};
R^{4a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁴ is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl;
more preferably, R⁴ is methyl, ethyl, n-propyl, iso-propyl, n-butyl, sec-butyl, iso-butyl, tert-butyl, cyclopropyl, cyclobutyl, cyclopentyl or cyclohexyl.

16. The compound of anyone of the preceding claims, wherein R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5f};
R^{5d} and R^{5f} are each independently selected from hydrogen, —F, —Cl, —Br, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl;
more preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl.

17. The compound of anyone of the preceding claims, wherein R⁵, R⁶ and R⁷ are each independently selected from H, —F, —Cl, —Br, —I, methyl, ethyl, propyl, butyl, pentyl, hexyl, heptyl or octyl; and/or
R⁸ is selected from —F, —Cl, —Br or —I; and/or
R⁹ is selected from H;
preferably, R⁵, R⁶ and R⁷ are each independently selected from H, —F, —Cl, methyl, ethyl, propyl or butyl; and/or
R⁸ is selected from —F or —Cl; and/or
R⁹ is selected from H.

18. The compound of anyone of the preceding claims, wherein R¹¹ is selected from H, methyl, ethyl, propyl, butyl, pentyl, hexyl, heptyl, octyl, —C₂₋₈alkenyl or —C₂₋₈alkynyl; wherein each of said methyl, ethyl, propyl, butyl, pentyl, hexyl, heptyl, octyl, —C₂₋₈alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R¹¹ is selected from H, methyl, ethyl, propyl, butyl, pentyl, hexyl, heptyl, octyl, —C₂₋₈alkenyl or —C₂₋₈alkynyl;
more preferably, R¹¹ is selected from H, methyl, ethyl, propyl or butyl;
even more preferably, R¹¹ is H.

19. The compound of anyone of the preceding claims, wherein the compound is selected from

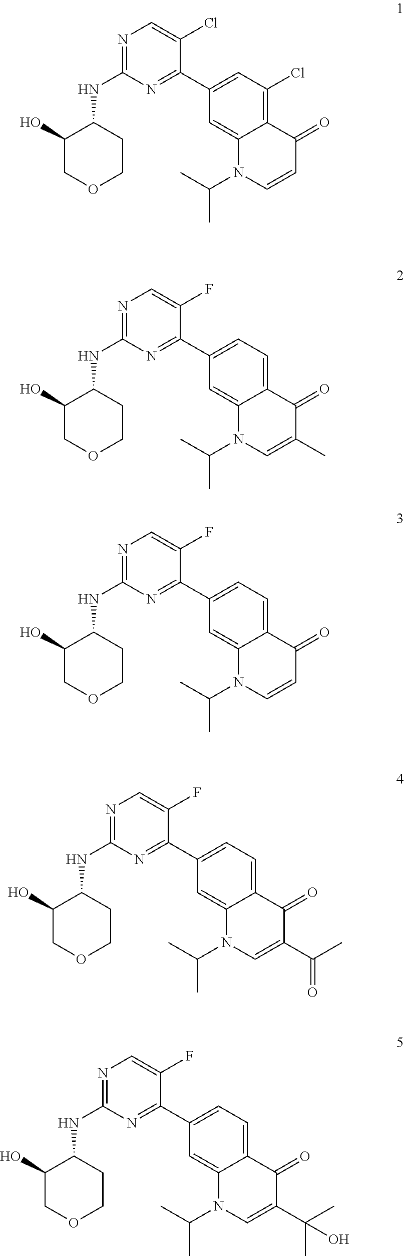

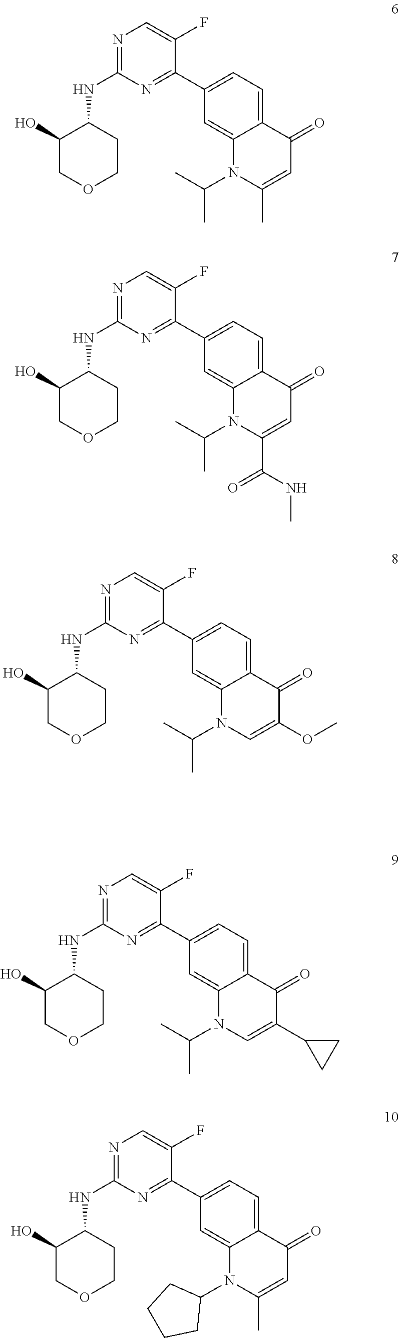 [SMILES: Cc1cc(=O)c2ccc(-c3nc(N[C@@H]4CCOC[C@H]4O)ncc3F)cc2n1C(C)C ; CNC(=O)c1cc(=O)c2ccc(-c3nc(N[C@@H]4CCOC[C@H]4O)ncc3F)cc2n1C(C)C ; COc1cn(C(C)C)c2cc(-c3nc(N[C@@H]4CCOC[C@H]4O)ncc3F)ccc2c1=O ; CC(C)n1cc(C2CC2)c(=O)c2ccc(-c3nc(N[C@@H]4CCOC[C@H]4O)ncc3F)cc21 ; Cc1cc(=O)c2ccc(-c3nc(N[C@@H]4CCOC[C@H]4O)ncc3F)cc2n1C1CCCC1]

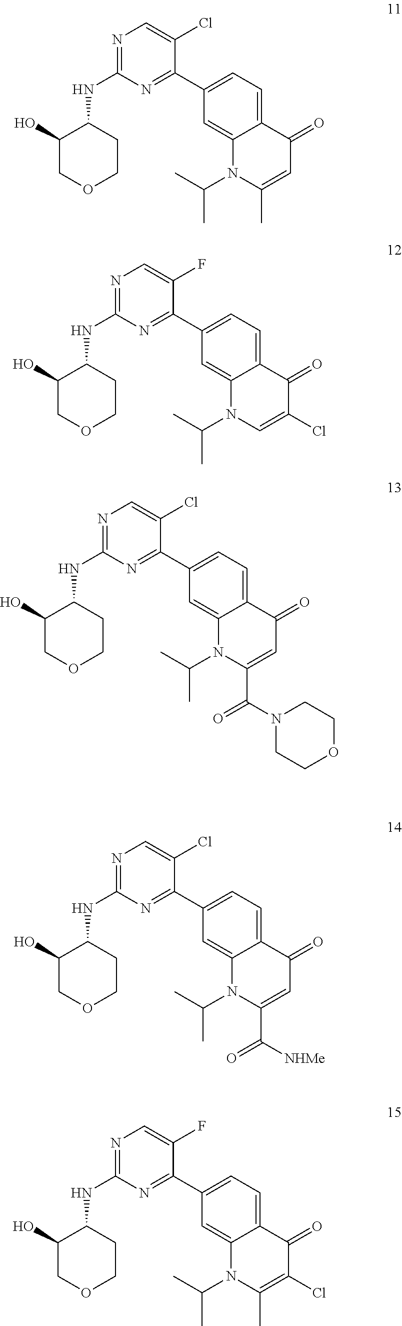

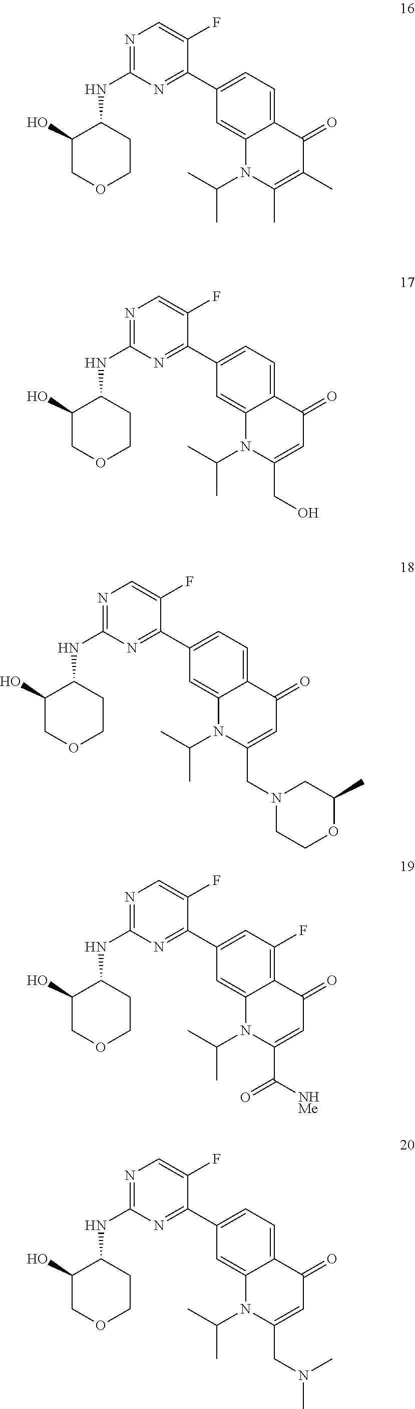 [SMILES: Cc1c(C)n(C(C)C)c2cc(-c3nc(N[C@@H]4CCOC[C@H]4O)ncc3F)ccc2c1=O ; CC(C)n1c(CO)cc(=O)c2ccc(-c3nc(N[C@@H]4CCOC[C@H]4O)ncc3F)cc21 ; CC(C)n1c(CN2CCO[C@H](C)C2)cc(=O)c2ccc(-c3nc(N[C@@H]4CCOC[C@H]4O)ncc3F)cc21 ; CNC(=O)c1cc(=O)c2c(F)cc(-c3nc(N[C@@H]4CCOC[C@H]4O)ncc3F)cc2n1C(C)C ; CC(C)n1c(CN(C)C)cc(=O)c2ccc(-c3nc(N[C@@H]4CCOC[C@H]4O)ncc3F)cc21]

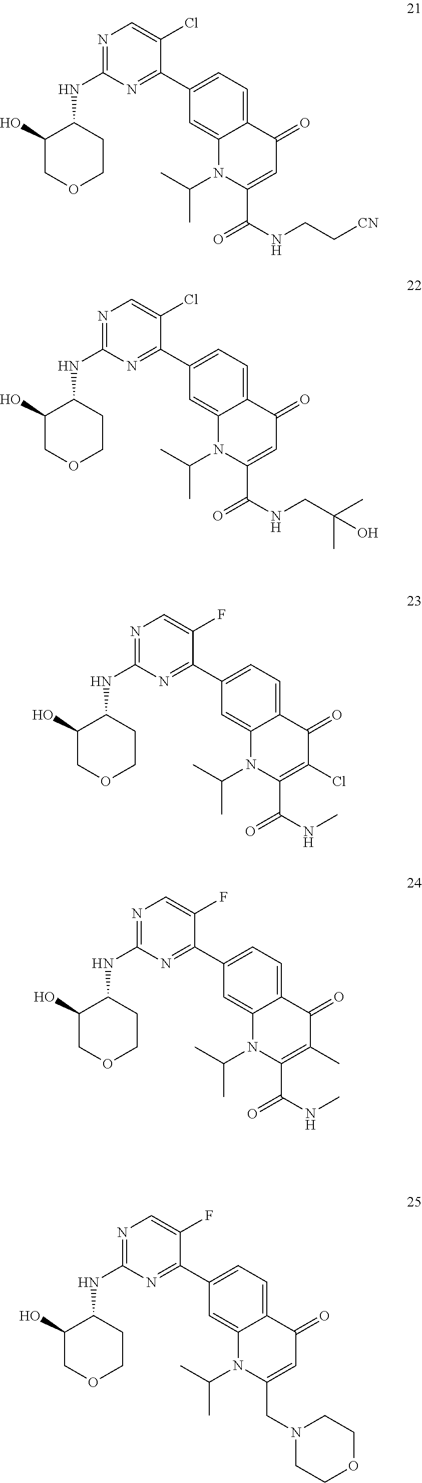 [SMILES: CC(C)n1c(C(=O)NCCC#N)cc(=O)c2ccc(-c3nc(N[C@@H]4CCOC[C@H]4O)ncc3Cl)cc21 ; CC(C)n1c(C(=O)NCC(C)(C)O)cc(=O)c2ccc(-c3nc(N[C@@H]4CCOC[C@H]4O)ncc3Cl)cc21 ; CNC(=O)c1c(Cl)c(=O)c2ccc(-c3nc(N[C@@H]4CCOC[C@H]4O)ncc3F)cc2n1C(C)C ; CNC(=O)c1c(C)c(=O)c2ccc(-c3nc(N[C@@H]4CCOC[C@H]4O)ncc3F)cc2n1C(C)C ; CC(C)n1c(CN2CCOCC2)cc(=O)c2ccc(-c3nc(N[C@@H]4CCOC[C@H]4O)ncc3F)cc21]

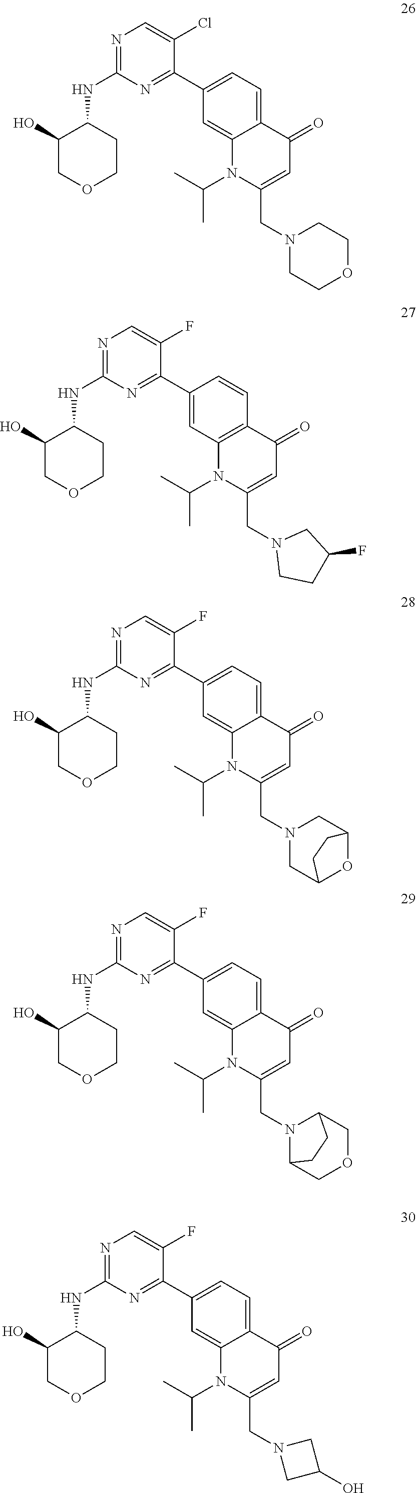 [SMILES: CC(C)n1c(CN2CCOCC2)cc(=O)c2ccc(-c3nc(N[C@@H]4CCOC[C@H]4O)ncc3Cl)cc21 ; CC(C)n1c(CN2CC[C@H](F)C2)cc(=O)c2ccc(-c3nc(N[C@@H]4CCOC[C@H]4O)ncc3F)cc21 ; CC(C)n1c(CN2CC3CCC(C2)O3)cc(=O)c2ccc(-c3nc(N[C@@H]4CCOC[C@H]4O)ncc3F)cc21 ; CC(C)n1c(CN2C3CCC2COC3)cc(=O)c2ccc(-c3nc(N[C@@H]4CCOC[C@H]4O)ncc3F)cc21 ; CC(C)n1c(CN2CC(O)C2)cc(=O)c2ccc(-c3nc(N[C@@H]4CCOC[C@H]4O)ncc3F)cc21]

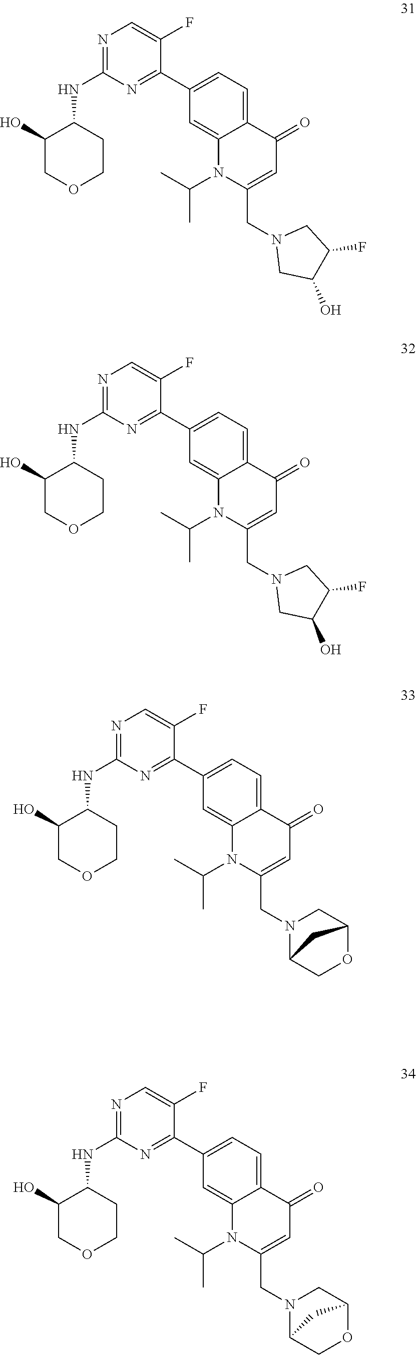 [SMILES: CC(C)n1c(CN2C[C@@H](O)[C@@H](F)C2)cc(=O)c2ccc(-c3nc(N[C@@H]4CCOC[C@H]4O)ncc3F)cc21 ; CC(C)n1c(CN2C[C@H](O)[C@@H](F)C2)cc(=O)c2ccc(-c3nc(N[C@@H]4CCOC[C@H]4O)ncc3F)cc21 ; CC(C)n1c(CN2C[C@H]3C[C@@H]2CO3)cc(=O)c2ccc(-c3nc(N[C@@H]4CCOC[C@H]4O)ncc3F)cc21 ; CC(C)n1c(CN2C[C@@H]3C[C@H]2CO3)cc(=O)c2ccc(-c3nc(N[C@@H]4CCOC[C@H]4O)ncc3F)cc21]

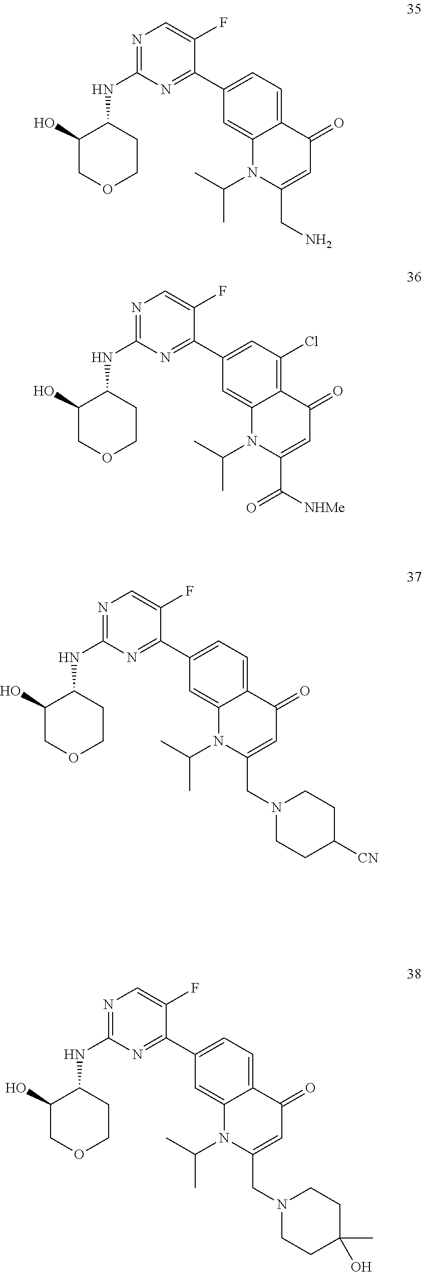 [SMILES: CC(C)n1c(CN)cc(=O)c2ccc(-c3nc(N[C@@H]4CCOC[C@H]4O)ncc3F)cc21 ; CNC(=O)c1cc(=O)c2c(Cl)cc(-c3nc(N[C@@H]4CCOC[C@H]4O)ncc3F)cc2n1C(C)C ; CC(C)n1c(CN2CCC(C#N)CC2)cc(=O)c2ccc(-c3nc(N[C@@H]4CCOC[C@H]4O)ncc3F)cc21 ; CC(C)n1c(CN2CCC(C)(O)CC2)cc(=O)c2ccc(-c3nc(N[C@@H]4CCOC[C@H]4O)ncc3F)cc21]

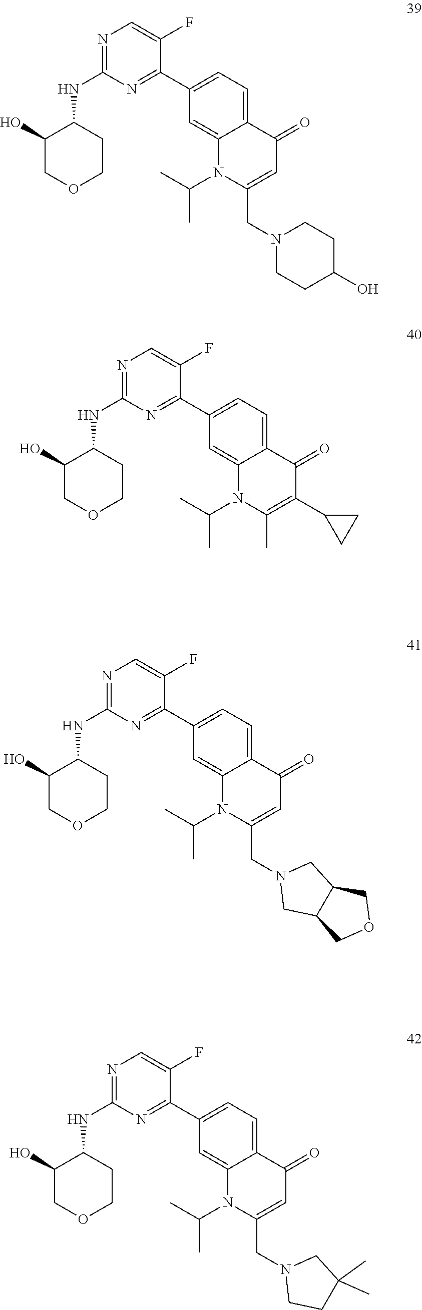 [SMILES: CC(C)n1c(CN2CCC(O)CC2)cc(=O)c2ccc(-c3nc(N[C@@H]4CCOC[C@H]4O)ncc3F)cc21 ; Cc1c(C2CC2)c(=O)c2ccc(-c3nc(N[C@@H]4CCOC[C@H]4O)ncc3F)cc2n1C(C)C ; CC(C)n1c(CN2C[C@H]3COC[C@H]3C2)cc(=O)c2ccc(-c3nc(N[C@@H]4CCOC[C@H]4O)ncc3F)cc21 ; CC(C)n1c(CN2CCC(C)(C)C2)cc(=O)c2ccc(-c3nc(N[C@@H]4CCOC[C@H]4O)ncc3F)cc21]

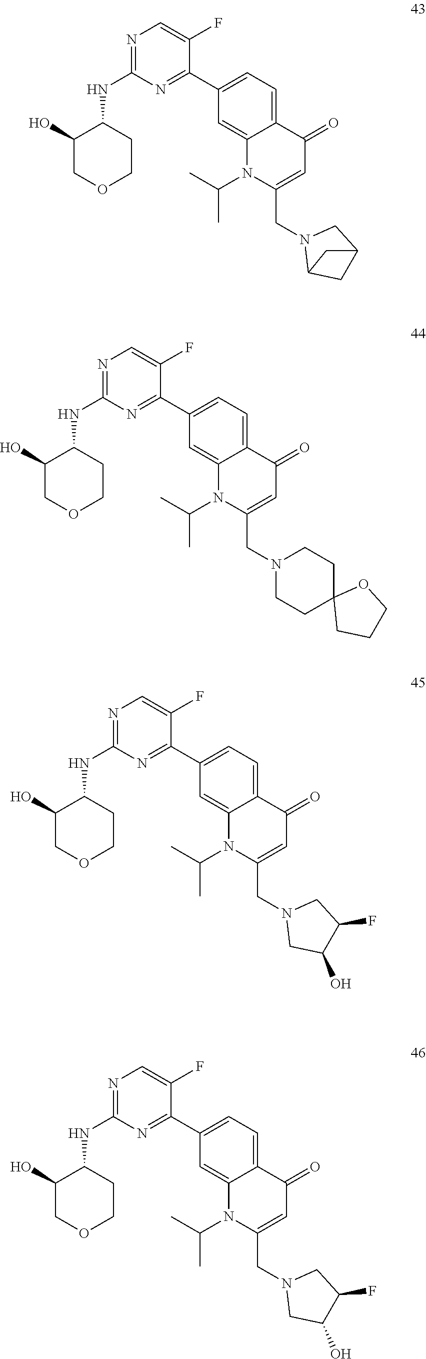 [SMILES: CC(C)n1c(CN2CC3CC2C3)cc(=O)c2ccc(-c3nc(N[C@@H]4CCOC[C@H]4O)ncc3F)cc21 ; CC(C)n1c(CN2CCC3(CCCO3)CC2)cc(=O)c2ccc(-c3nc(N[C@@H]4CCOC[C@H]4O)ncc3F)cc21 ; CC(C)n1c(CN2C[C@@H](F)[C@@H](O)C2)cc(=O)c2ccc(-c3nc(N[C@@H]4CCOC[C@H]4O)ncc3F)cc21 ; CC(C)n1c(CN2C[C@@H](O)[C@H](F)C2)cc(=O)c2ccc(-c3nc(N[C@@H]4CCOC[C@H]4O)ncc3F)cc21]

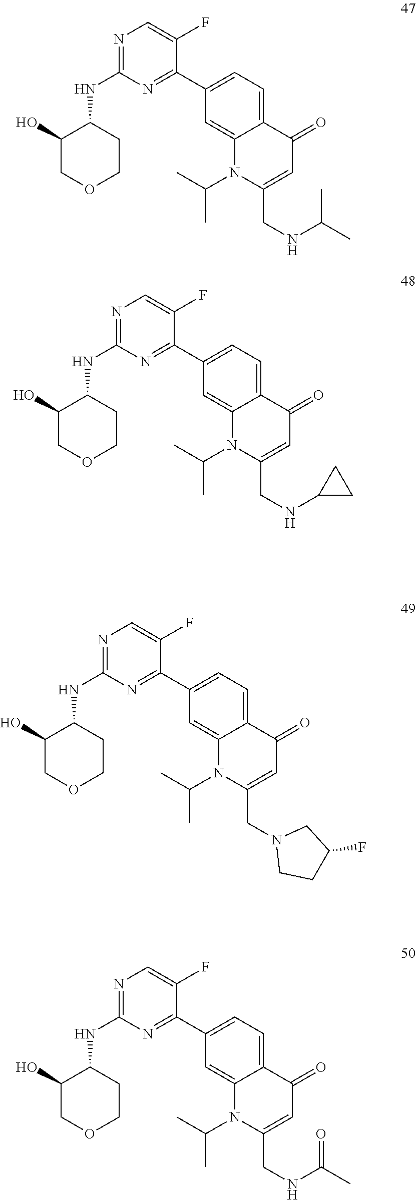 [SMILES: CC(C)NCc1cc(=O)c2ccc(-c3nc(N[C@@H]4CCOC[C@H]4O)ncc3F)cc2n1C(C)C ; CC(C)n1c(CNC2CC2)cc(=O)c2ccc(-c3nc(N[C@@H]4CCOC[C@H]4O)ncc3F)cc21 ; CC(C)n1c(CN2CC[C@@H](F)C2)cc(=O)c2ccc(-c3nc(N[C@@H]4CCOC[C@H]4O)ncc3F)cc21 ; CC(=O)NCc1cc(=O)c2ccc(-c3nc(N[C@@H]4CCOC[C@H]4O)ncc3F)cc2n1C(C)C]

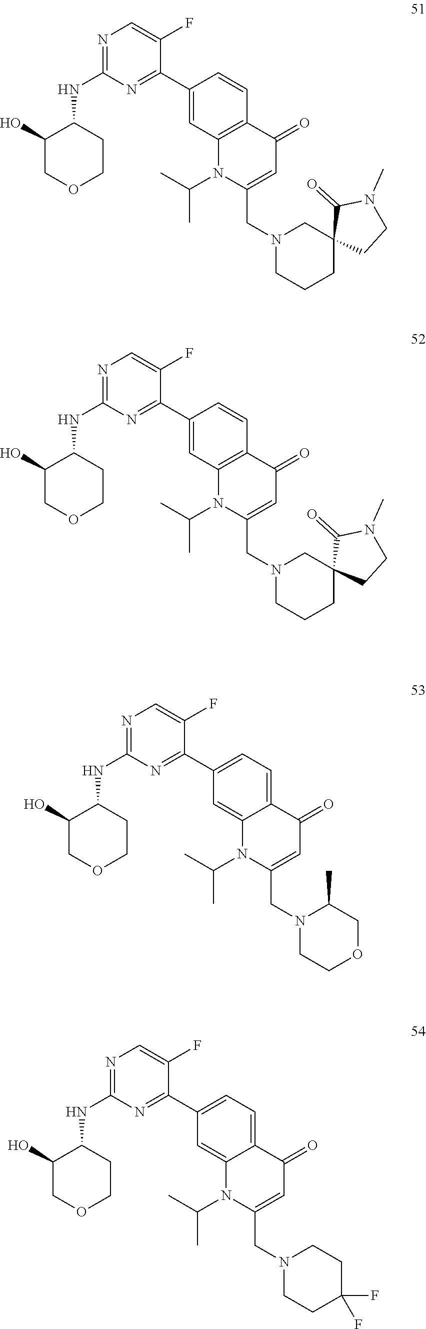 [SMILES: CC(C)n1c(CN2CCC[C@@]3(CCN(C)C3=O)C2)cc(=O)c2ccc(-c3nc(N[C@@H]4CCOC[C@H]4O)ncc3F)cc21 ; CC(C)n1c(CN2CCC[C@]3(CCN(C)C3=O)C2)cc(=O)c2ccc(-c3nc(N[C@@H]4CCOC[C@H]4O)ncc3F)cc21 ; CC(C)n1c(CN2CCOC[C@@H]2C)cc(=O)c2ccc(-c3nc(N[C@@H]4CCOC[C@H]4O)ncc3F)cc21 ; CC(C)n1c(CN2CCC(F)(F)CC2)cc(=O)c2ccc(-c3nc(N[C@@H]4CCOC[C@H]4O)ncc3F)cc21]

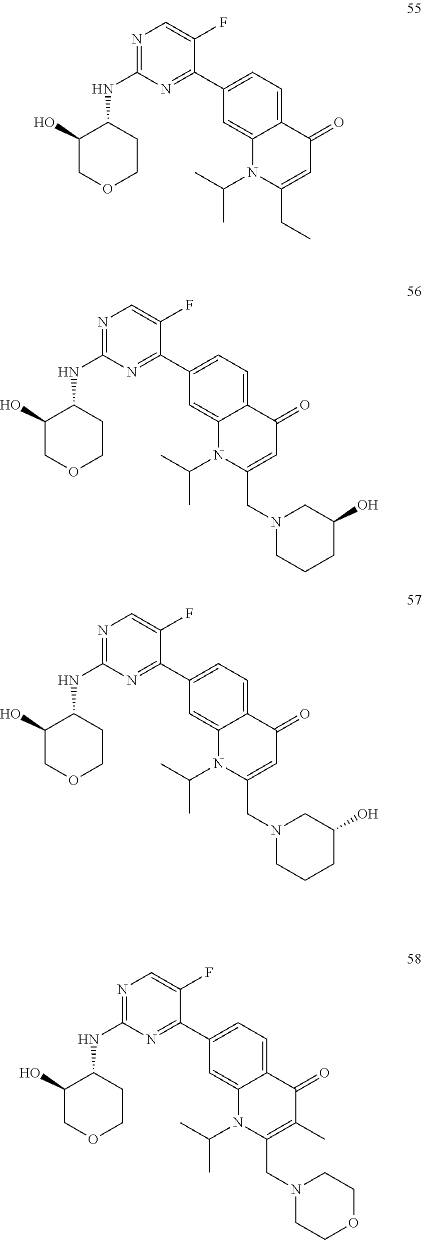 [SMILES: CCc1cc(=O)c2ccc(-c3nc(N[C@@H]4CCOC[C@H]4O)ncc3F)cc2n1C(C)C ; CC(C)n1c(CN2CCC[C@H](O)C2)cc(=O)c2ccc(-c3nc(N[C@@H]4CCOC[C@H]4O)ncc3F)cc21 ; CC(C)n1c(CN2CCC[C@@H](O)C2)cc(=O)c2ccc(-c3nc(N[C@@H]4CCOC[C@H]4O)ncc3F)cc21 ; Cc1c(CN2CCOCC2)n(C(C)C)c2cc(-c3nc(N[C@@H]4CCOC[C@H]4O)ncc3F)ccc2c1=O]

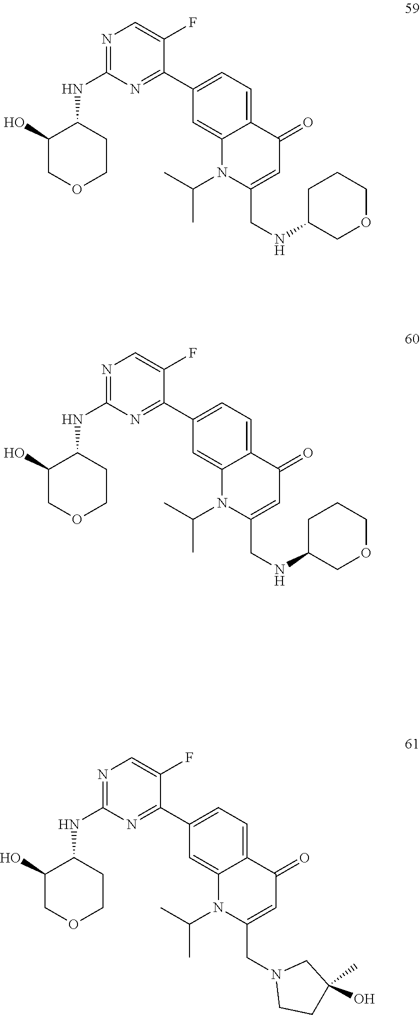 [SMILES: CC(C)n1c(CN[C@@H]2CCCOC2)cc(=O)c2ccc(-c3nc(N[C@@H]4CCOC[C@H]4O)ncc3F)cc21 ; CC(C)n1c(CN[C@H]2CCCOC2)cc(=O)c2ccc(-c3nc(N[C@@H]4CCOC[C@H]4O)ncc3F)cc21 ; CC(C)n1c(CN2CC[C@](C)(O)C2)cc(=O)c2ccc(-c3nc(N[C@@H]4CCOC[C@H]4O)ncc3F)cc21]

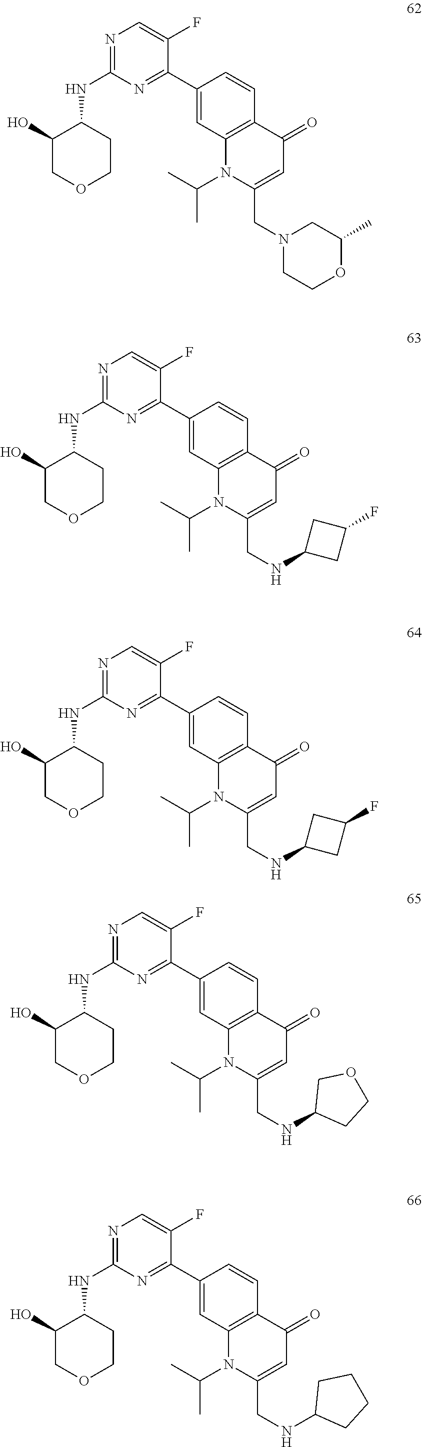 [SMILES: CC(C)n1c(CN2CCO[C@@H](C)C2)cc(=O)c2ccc(-c3nc(N[C@@H]4CCOC[C@H]4O)ncc3F)cc21 ; CC(C)n1c(CN[C@H]2C[C@H](F)C2)cc(=O)c2ccc(-c3nc(N[C@@H]4CCOC[C@H]4O)ncc3F)cc21 ; CC(C)n1c(CN[C@H]2C[C@@H](F)C2)cc(=O)c2ccc(-c3nc(N[C@@H]4CCOC[C@H]4O)ncc3F)cc21 ; CC(C)n1c(CN[C@@H]2CCOC2)cc(=O)c2ccc(-c3nc(N[C@@H]4CCOC[C@H]4O)ncc3F)cc21 ; CC(C)n1c(CNC2CCCC2)cc(=O)c2ccc(-c3nc(N[C@@H]4CCOC[C@H]4O)ncc3F)cc21]

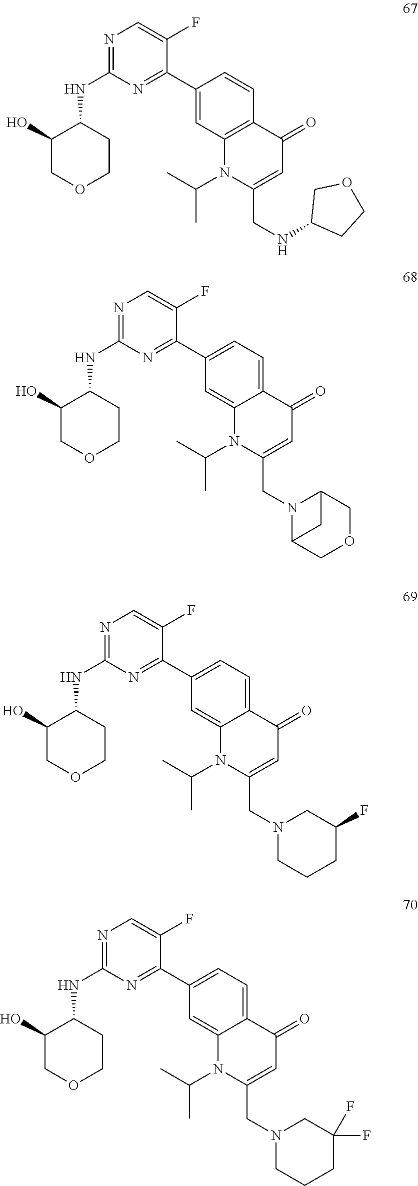 [SMILES: CC(C)n1c(CN[C@H]2CCOC2)cc(=O)c2ccc(-c3nc(N[C@@H]4CCOC[C@H]4O)ncc3F)cc21 ; CC(C)n1c(CN2C3COCC2C3)cc(=O)c2ccc(-c3nc(N[C@@H]4CCOC[C@H]4O)ncc3F)cc21 ; CC(C)n1c(CN2CCC[C@H](F)C2)cc(=O)c2ccc(-c3nc(N[C@@H]4CCOC[C@H]4O)ncc3F)cc21 ; CC(C)n1c(CN2CCCC(F)(F)C2)cc(=O)c2ccc(-c3nc(N[C@@H]4CCOC[C@H]4O)ncc3F)cc21]

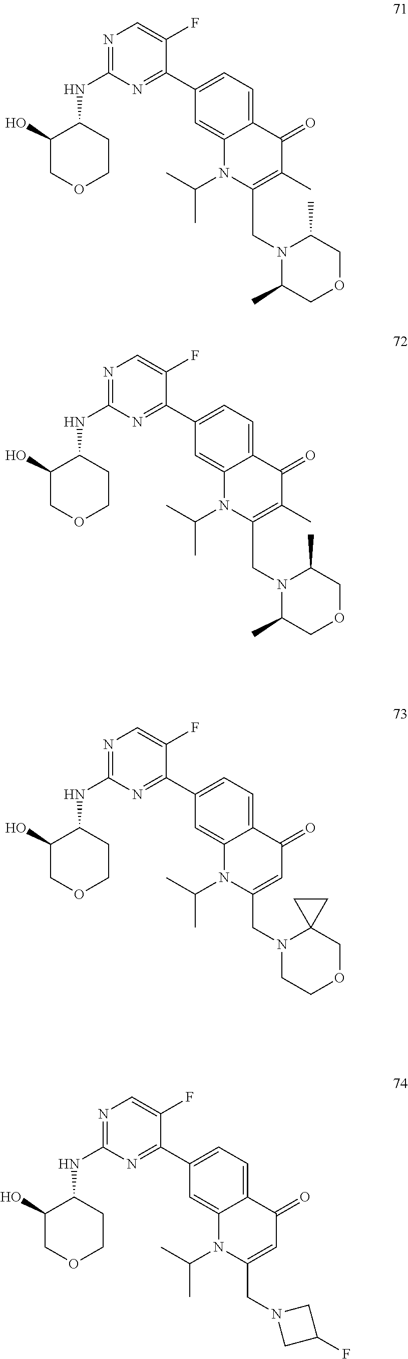 [SMILES: Cc1c(CN2[C@H](C)COC[C@H]2C)n(C(C)C)c2cc(-c3nc(N[C@@H]4CCOC[C@H]4O)ncc3F)ccc2c1=O ; Cc1c(CN2[C@H](C)COC[C@@H]2C)n(C(C)C)c2cc(-c3nc(N[C@@H]4CCOC[C@H]4O)ncc3F)ccc2c1=O ; CC(C)n1c(CN2CCOCC23CC3)cc(=O)c2ccc(-c3nc(N[C@@H]4CCOC[C@H]4O)ncc3F)cc21 ; CC(C)n1c(CN2CC(F)C2)cc(=O)c2ccc(-c3nc(N[C@@H]4CCOC[C@H]4O)ncc3F)cc21]

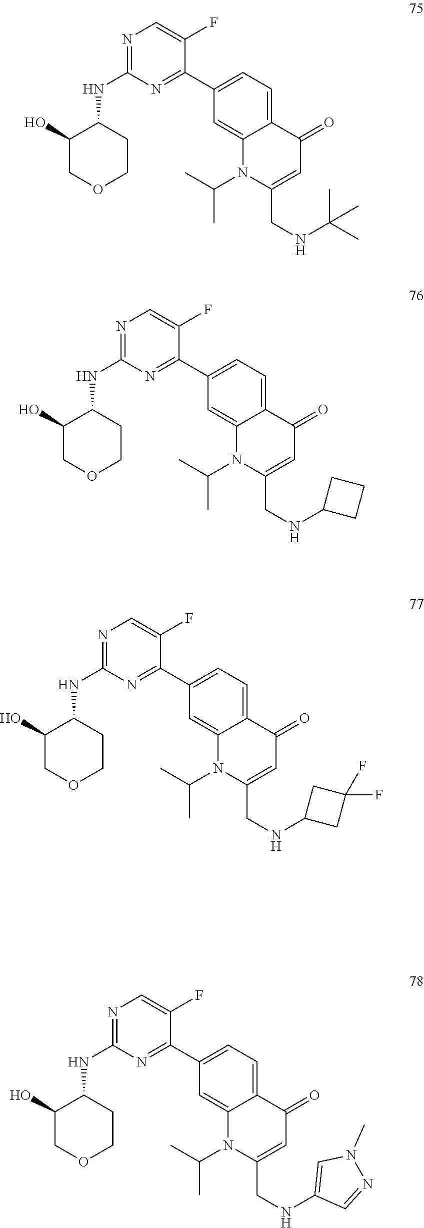 [SMILES: CC(C)n1c(CNC(C)(C)C)cc(=O)c2ccc(-c3nc(N[C@@H]4CCOC[C@H]4O)ncc3F)cc21 ; CC(C)n1c(CNC2CCC2)cc(=O)c2ccc(-c3nc(N[C@@H]4CCOC[C@H]4O)ncc3F)cc21 ; CC(C)n1c(CNC2CC(F)(F)C2)cc(=O)c2ccc(-c3nc(N[C@@H]4CCOC[C@H]4O)ncc3F)cc21 ; CC(C)n1c(CNc2cnn(C)c2)cc(=O)c2ccc(-c3nc(N[C@@H]4CCOC[C@H]4O)ncc3F)cc21]

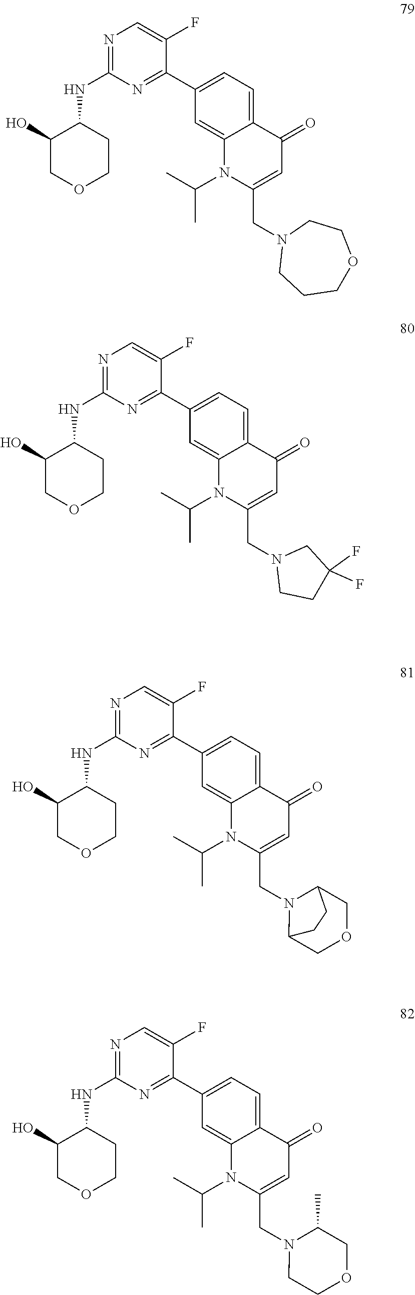 [SMILES: CC(C)n1c(CN2CCCOCC2)cc(=O)c2ccc(-c3nc(N[C@@H]4CCOC[C@H]4O)ncc3F)cc21 ; CC(C)n1c(CN2CCC(F)(F)C2)cc(=O)c2ccc(-c3nc(N[C@@H]4CCOC[C@H]4O)ncc3F)cc21 ; CC(C)n1c(CN2C3CCC2COC3)cc(=O)c2ccc(-c3nc(N[C@@H]4CCOC[C@H]4O)ncc3F)cc21 ; CC(C)n1c(CN2CCOC[C@H]2C)cc(=O)c2ccc(-c3nc(N[C@@H]4CCOC[C@H]4O)ncc3F)cc21]

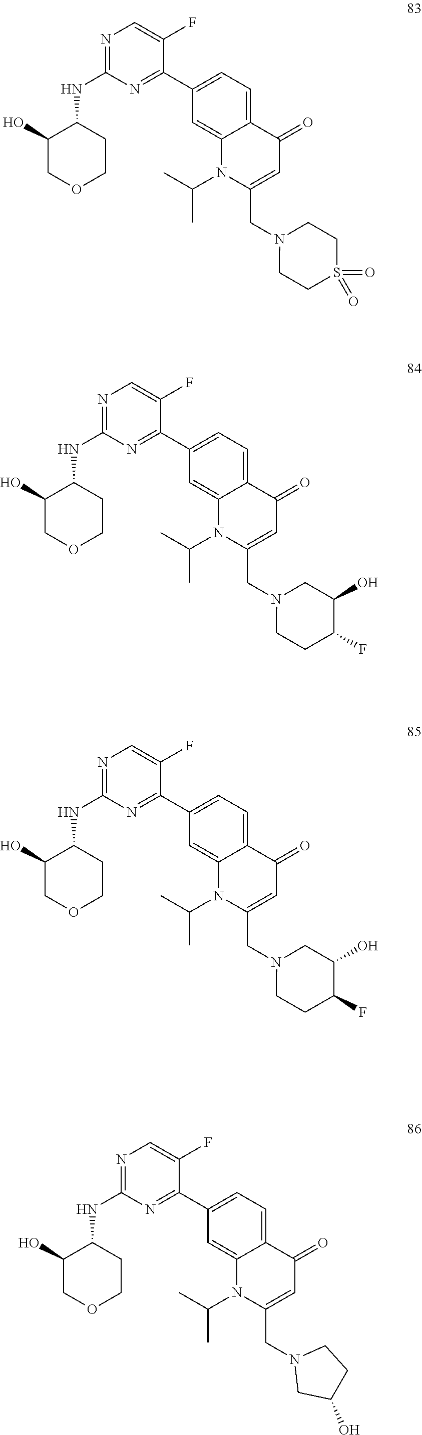 [SMILES: CC(C)n1c(CN2CCS(=O)(=O)CC2)cc(=O)c2ccc(-c3nc(N[C@@H]4CCOC[C@H]4O)ncc3F)cc21 ; CC(C)n1c(CN2CC[C@@H](F)[C@H](O)C2)cc(=O)c2ccc(-c3nc(N[C@@H]4CCOC[C@H]4O)ncc3F)cc21 ; CC(C)n1c(CN2CC[C@H](F)[C@@H](O)C2)cc(=O)c2ccc(-c3nc(N[C@@H]4CCOC[C@H]4O)ncc3F)cc21 ; CC(C)n1c(CN2CC[C@H](O)C2)cc(=O)c2ccc(-c3nc(N[C@@H]4CCOC[C@H]4O)ncc3F)cc21]

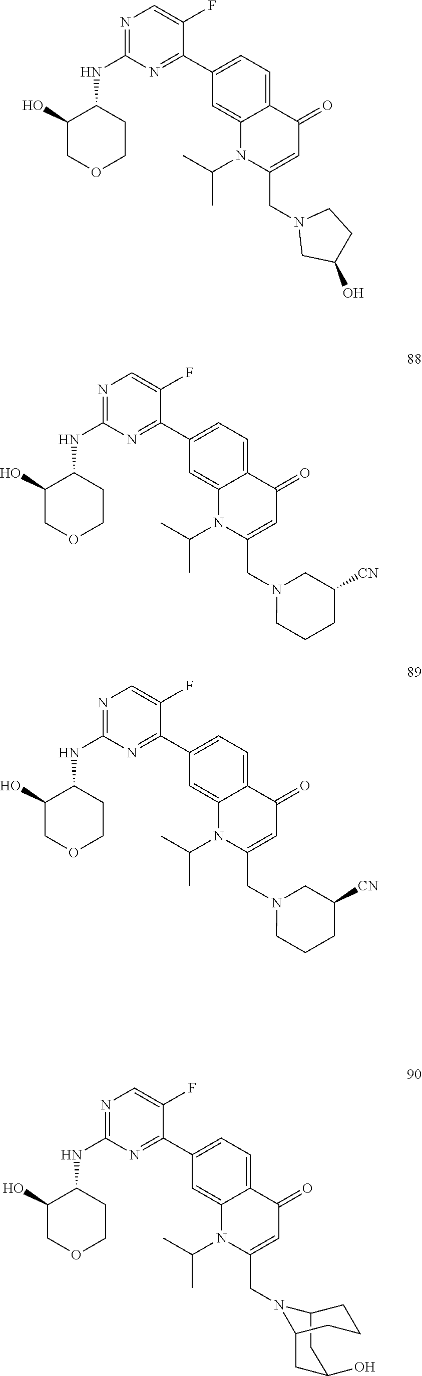 [SMILES: CC(C)n1c(CN2CC[C@@H](O)C2)cc(=O)c2ccc(-c3nc(N[C@@H]4CCOC[C@H]4O)ncc3F)cc21 ; CC(C)n1c(CN2CCC[C@@H](C#N)C2)cc(=O)c2ccc(-c3nc(N[C@@H]4CCOC[C@H]4O)ncc3F)cc21 ; CC(C)n1c(CN2CCC[C@H](C#N)C2)cc(=O)c2ccc(-c3nc(N[C@@H]4CCOC[C@H]4O)ncc3F)cc21 ; CC(C)n1c(CN2C3CCCC2CC(O)C3)cc(=O)c2ccc(-c3nc(N[C@@H]4CCOC[C@H]4O)ncc3F)cc21]

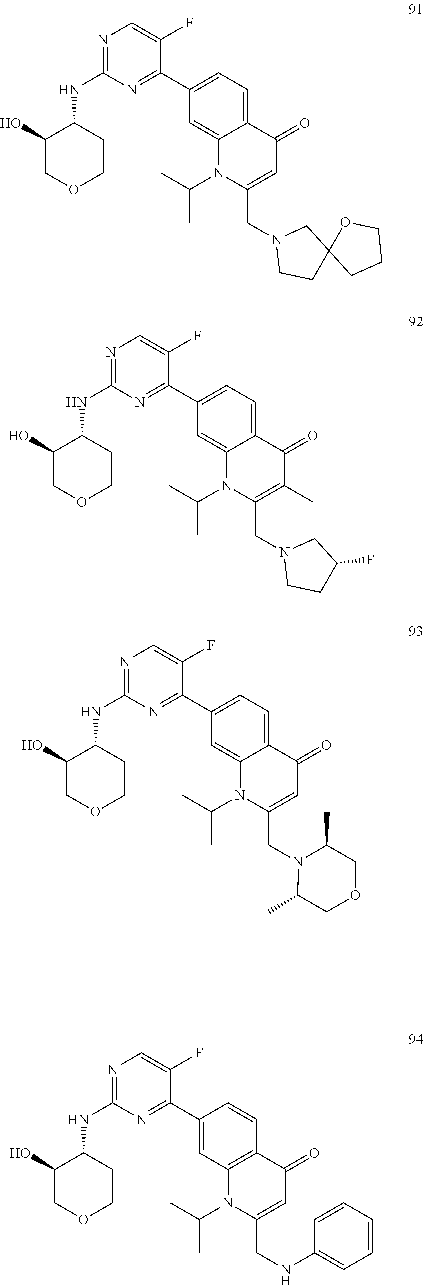 [SMILES: CC(C)n1c(CN2CCC3(CCCO3)C2)cc(=O)c2ccc(-c3nc(N[C@@H]4CCOC[C@H]4O)ncc3F)cc21 ; Cc1c(CN2CC[C@@H](F)C2)n(C(C)C)c2cc(-c3nc(N[C@@H]4CCOC[C@H]4O)ncc3F)ccc2c1=O ; CC(C)n1c(CN2[C@@H](C)COC[C@@H]2C)cc(=O)c2ccc(-c3nc(N[C@@H]4CCOC[C@H]4O)ncc3F)cc21 ; CC(C)n1c(CNc2ccccc2)cc(=O)c2ccc(-c3nc(N[C@@H]4CCOC[C@H]4O)ncc3F)cc21]

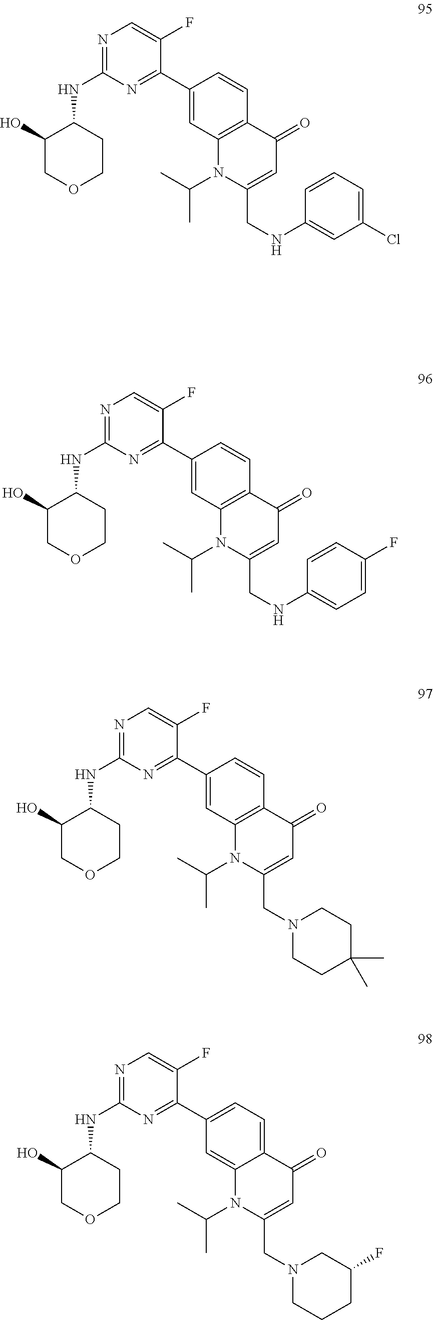 [SMILES: CC(C)n1c(CNc2cccc(Cl)c2)cc(=O)c2ccc(-c3nc(N[C@@H]4CCOC[C@H]4O)ncc3F)cc21 ; CC(C)n1c(CNc2ccc(F)cc2)cc(=O)c2ccc(-c3nc(N[C@@H]4CCOC[C@H]4O)ncc3F)cc21 ; CC(C)n1c(CN2CCC(C)(C)CC2)cc(=O)c2ccc(-c3nc(N[C@@H]4CCOC[C@H]4O)ncc3F)cc21 ; CC(C)n1c(CN2CCC[C@@H](F)C2)cc(=O)c2ccc(-c3nc(N[C@@H]4CCOC[C@H]4O)ncc3F)cc21]

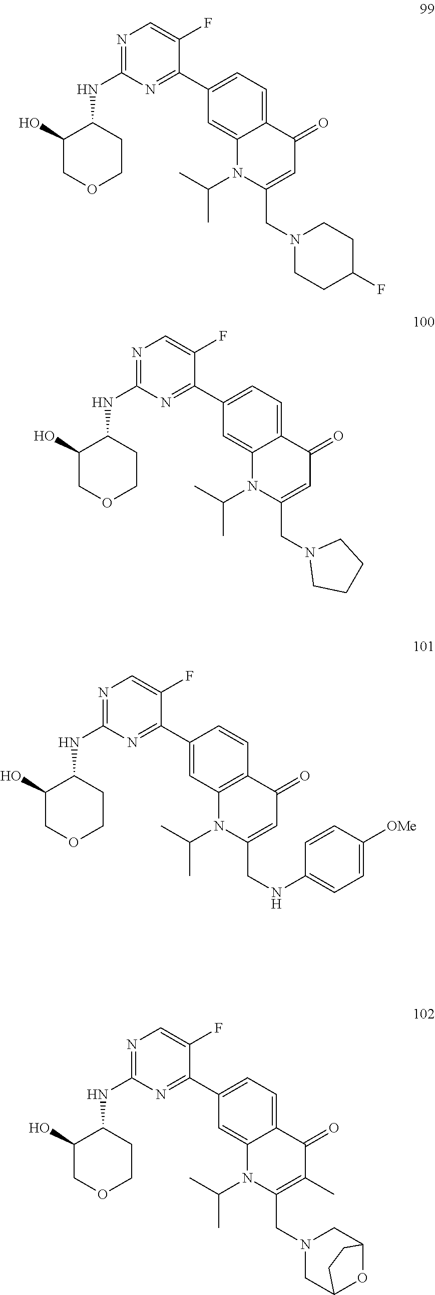 [SMILES: CC(C)n1c(CN2CCC(F)CC2)cc(=O)c2ccc(-c3nc(N[C@@H]4CCOC[C@H]4O)ncc3F)cc21 ; CC(C)n1c(CN2CCCC2)cc(=O)c2ccc(-c3nc(N[C@@H]4CCOC[C@H]4O)ncc3F)cc21 ; COc1ccc(NCc2cc(=O)c3ccc(-c4nc(N[C@@H]5CCOC[C@H]5O)ncc4F)cc3n2C(C)C)cc1 ; Cc1c(CN2CC3CCC(C2)O3)n(C(C)C)c2cc(-c3nc(N[C@@H]4CCOC[C@H]4O)ncc3F)ccc2c1=O]

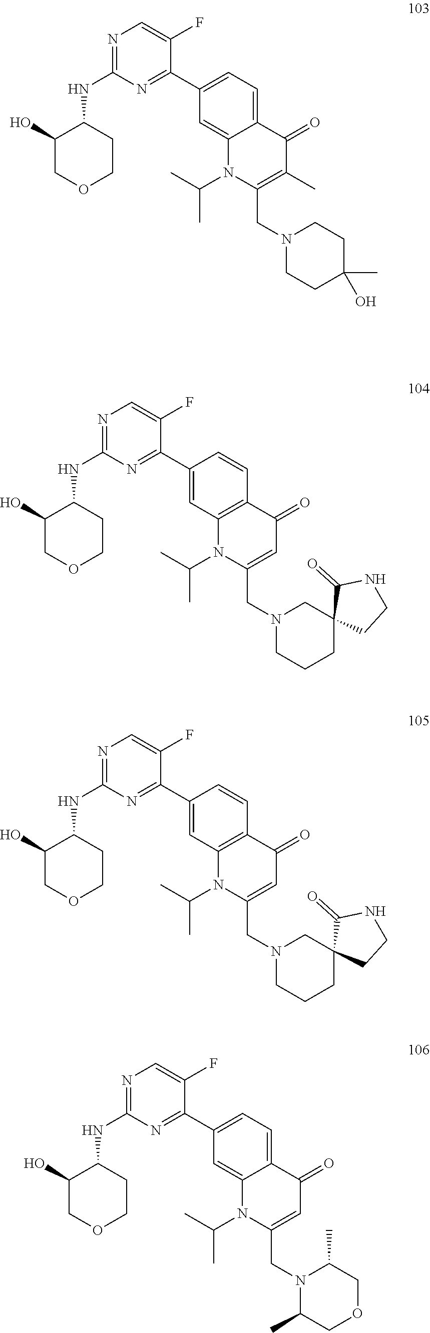 [SMILES: Cc1c(CN2CCC(C)(O)CC2)n(C(C)C)c2cc(-c3nc(N[C@@H]4CCOC[C@H]4O)ncc3F)ccc2c1=O ; CC(C)n1c(CN2CCC[C@@]3(CCNC3=O)C2)cc(=O)c2ccc(-c3nc(N[C@@H]4CCOC[C@H]4O)ncc3F)cc21 ; CC(C)n1c(CN2CCC[C@]3(CCNC3=O)C2)cc(=O)c2ccc(-c3nc(N[C@@H]4CCOC[C@H]4O)ncc3F)cc21 ; CC(C)n1c(CN2[C@H](C)COC[C@H]2C)cc(=O)c2ccc(-c3nc(N[C@@H]4CCOC[C@H]4O)ncc3F)cc21]

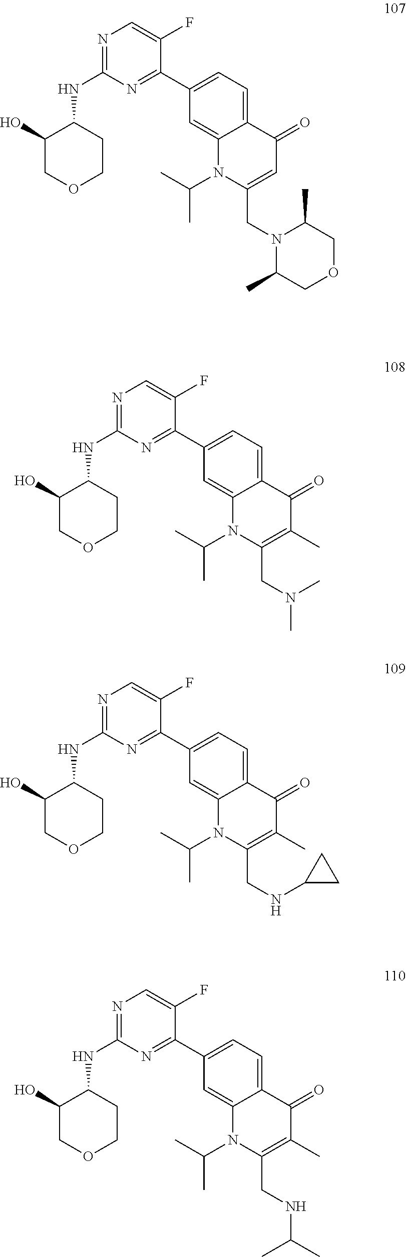 [SMILES: CC(C)n1c(CN2[C@H](C)COC[C@@H]2C)cc(=O)c2ccc(-c3nc(N[C@@H]4CCOC[C@H]4O)ncc3F)cc21 ; Cc1c(CN(C)C)n(C(C)C)c2cc(-c3nc(N[C@@H]4CCOC[C@H]4O)ncc3F)ccc2c1=O ; Cc1c(CNC2CC2)n(C(C)C)c2cc(-c3nc(N[C@@H]4CCOC[C@H]4O)ncc3F)ccc2c1=O ; Cc1c(CNC(C)C)n(C(C)C)c2cc(-c3nc(N[C@@H]4CCOC[C@H]4O)ncc3F)ccc2c1=O]

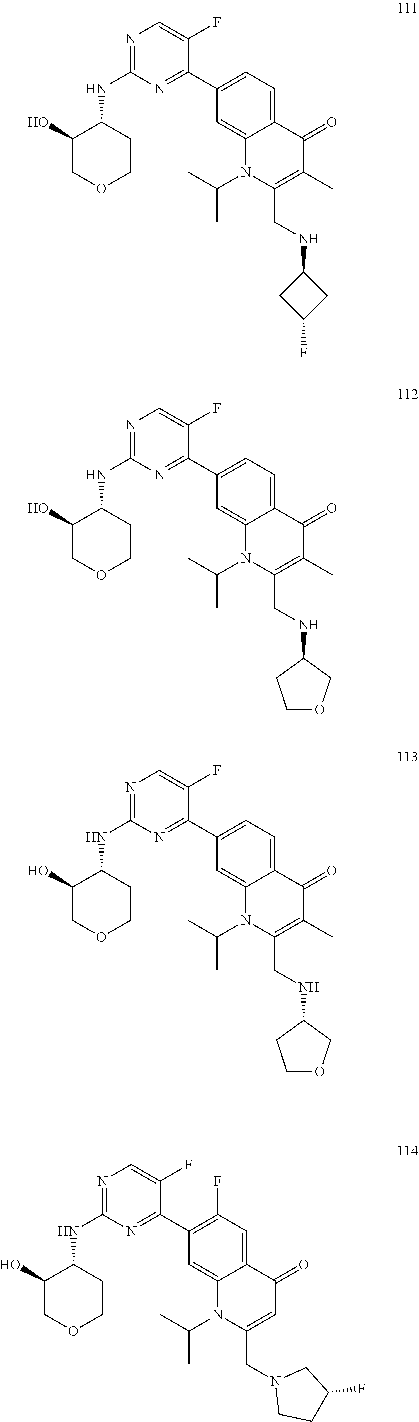 [SMILES: Cc1c(CN[C@H]2C[C@H](F)C2)n(C(C)C)c2cc(-c3nc(N[C@@H]4CCOC[C@H]4O)ncc3F)ccc2c1=O ; Cc1c(CN[C@@H]2CCOC2)n(C(C)C)c2cc(-c3nc(N[C@@H]4CCOC[C@H]4O)ncc3F)ccc2c1=O ; Cc1c(CN[C@H]2CCOC2)n(C(C)C)c2cc(-c3nc(N[C@@H]4CCOC[C@H]4O)ncc3F)ccc2c1=O ; CC(C)n1c(CN2CC[C@@H](F)C2)cc(=O)c2cc(F)c(-c3nc(N[C@@H]4CCOC[C@H]4O)ncc3F)cc21]

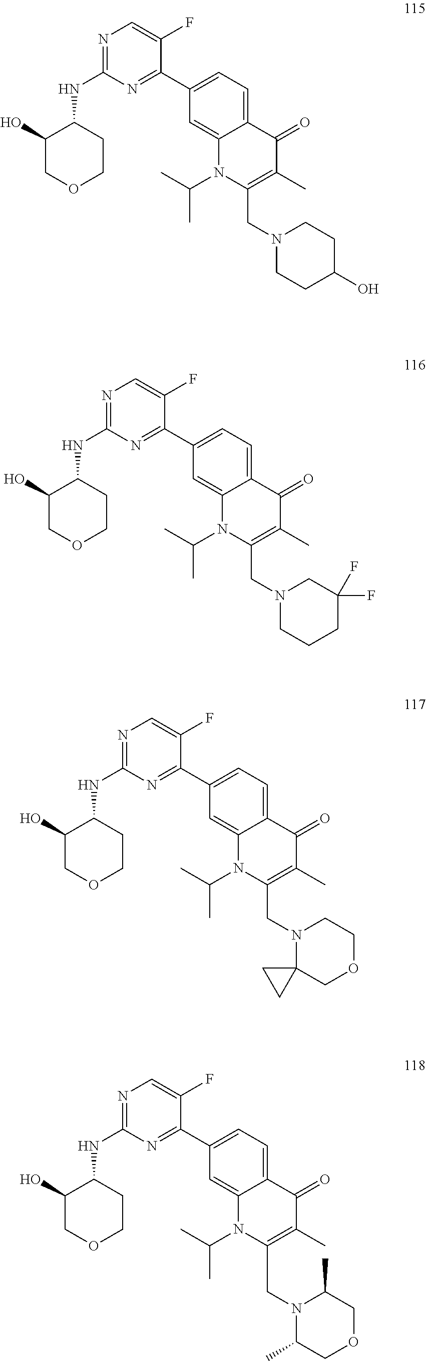 [SMILES: Cc1c(CN2CCC(O)CC2)n(C(C)C)c2cc(-c3nc(N[C@@H]4CCOC[C@H]4O)ncc3F)ccc2c1=O ; Cc1c(CN2CCCC(F)(F)C2)n(C(C)C)c2cc(-c3nc(N[C@@H]4CCOC[C@H]4O)ncc3F)ccc2c1=O ; Cc1c(CN2CCOCC23CC3)n(C(C)C)c2cc(-c3nc(N[C@@H]4CCOC[C@H]4O)ncc3F)ccc2c1=O ; Cc1c(CN2[C@@H](C)COC[C@@H]2C)n(C(C)C)c2cc(-c3nc(N[C@@H]4CCOC[C@H]4O)ncc3F)ccc2c1=O]

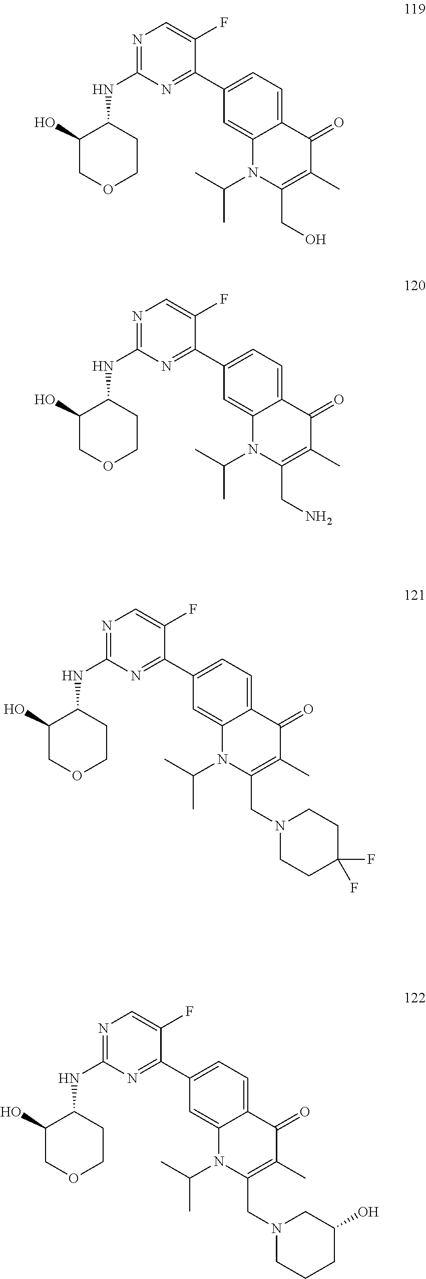 [SMILES: Cc1c(CO)n(C(C)C)c2cc(-c3nc(N[C@@H]4CCOC[C@H]4O)ncc3F)ccc2c1=O ; Cc1c(CN)n(C(C)C)c2cc(-c3nc(N[C@@H]4CCOC[C@H]4O)ncc3F)ccc2c1=O ; Cc1c(CN2CCC(F)(F)CC2)n(C(C)C)c2cc(-c3nc(N[C@@H]4CCOC[C@H]4O)ncc3F)ccc2c1=O ; Cc1c(CN2CCC[C@@H](O)C2)n(C(C)C)c2cc(-c3nc(N[C@@H]4CCOC[C@H]4O)ncc3F)ccc2c1=O]

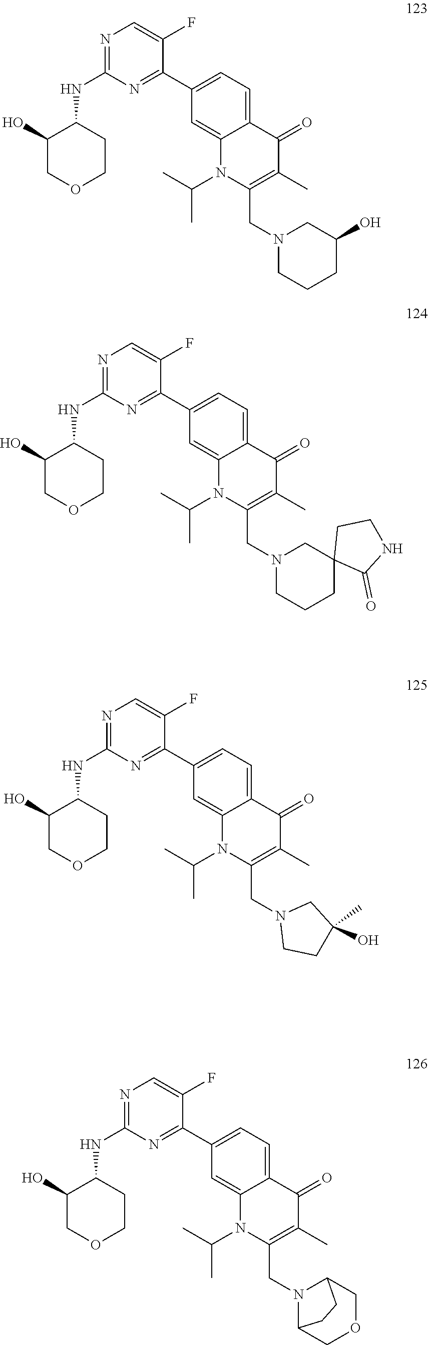 [SMILES: Cc1c(CN2CCC[C@H](O)C2)n(C(C)C)c2cc(-c3nc(N[C@@H]4CCOC[C@H]4O)ncc3F)ccc2c1=O ; Cc1c(CN2CCCC3(CCNC3=O)C2)n(C(C)C)c2cc(-c3nc(N[C@@H]4CCOC[C@H]4O)ncc3F)ccc2c1=O ; Cc1c(CN2CC[C@](C)(O)C2)n(C(C)C)c2cc(-c3nc(N[C@@H]4CCOC[C@H]4O)ncc3F)ccc2c1=O ; Cc1c(CN2C3CCC2COC3)n(C(C)C)c2cc(-c3nc(N[C@@H]4CCOC[C@H]4O)ncc3F)ccc2c1=O]

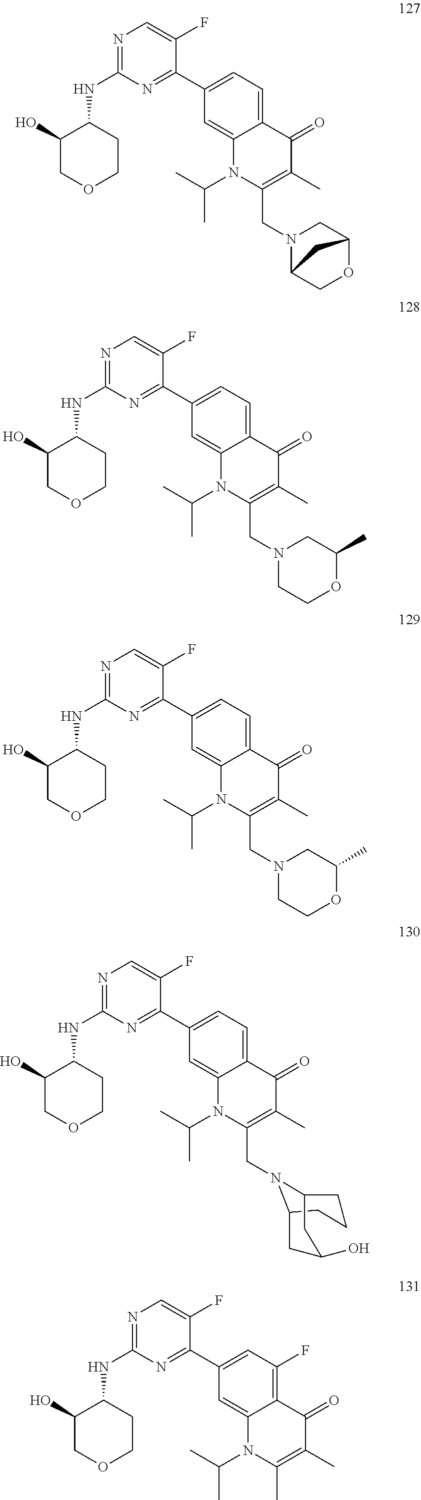 [SMILES: Cc1c(CN2C[C@H]3C[C@@H]2CO3)n(C(C)C)c2cc(-c3nc(N[C@@H]4CCOC[C@H]4O)ncc3F)ccc2c1=O ; Cc1c(CN2CCO[C@H](C)C2)n(C(C)C)c2cc(-c3nc(N[C@@H]4CCOC[C@H]4O)ncc3F)ccc2c1=O ; Cc1c(CN2CCO[C@@H](C)C2)n(C(C)C)c2cc(-c3nc(N[C@@H]4CCOC[C@H]4O)ncc3F)ccc2c1=O ; Cc1c(CN2C3CCCC2CC(O)C3)n(C(C)C)c2cc(-c3nc(N[C@@H]4CCOC[C@H]4O)ncc3F)ccc2c1=O ; Cc1c(C)n(C(C)C)c2cc(-c3nc(N[C@@H]4CCOC[C@H]4O)ncc3F)cc(F)c2c1=O]

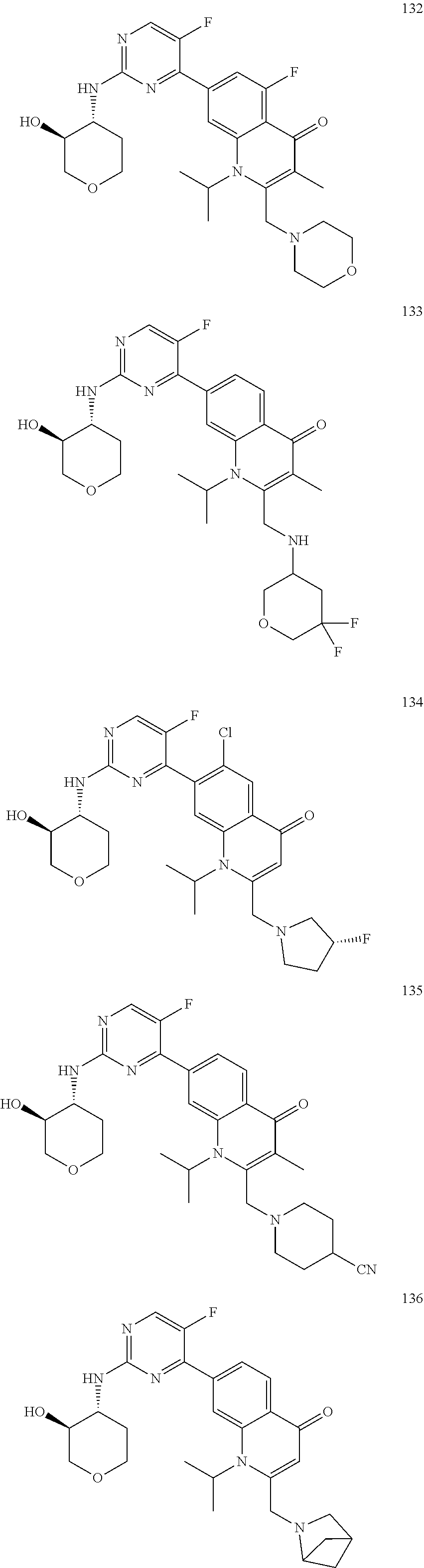 [SMILES: Cc1c(CN2CCOCC2)n(C(C)C)c2cc(-c3nc(N[C@@H]4CCOC[C@H]4O)ncc3F)cc(F)c2c1=O ; Cc1c(CNC2COCC(F)(F)C2)n(C(C)C)c2cc(-c3nc(N[C@@H]4CCOC[C@H]4O)ncc3F)ccc2c1=O ; CC(C)n1c(CN2CC[C@@H](F)C2)cc(=O)c2cc(Cl)c(-c3nc(N[C@@H]4CCOC[C@H]4O)ncc3F)cc21 ; Cc1c(CN2CCC(C#N)CC2)n(C(C)C)c2cc(-c3nc(N[C@@H]4CCOC[C@H]4O)ncc3F)ccc2c1=O ; CC(C)n1c(CN2CC3CC2C3)cc(=O)c2ccc(-c3nc(N[C@@H]4CCOC[C@H]4O)ncc3F)cc21]

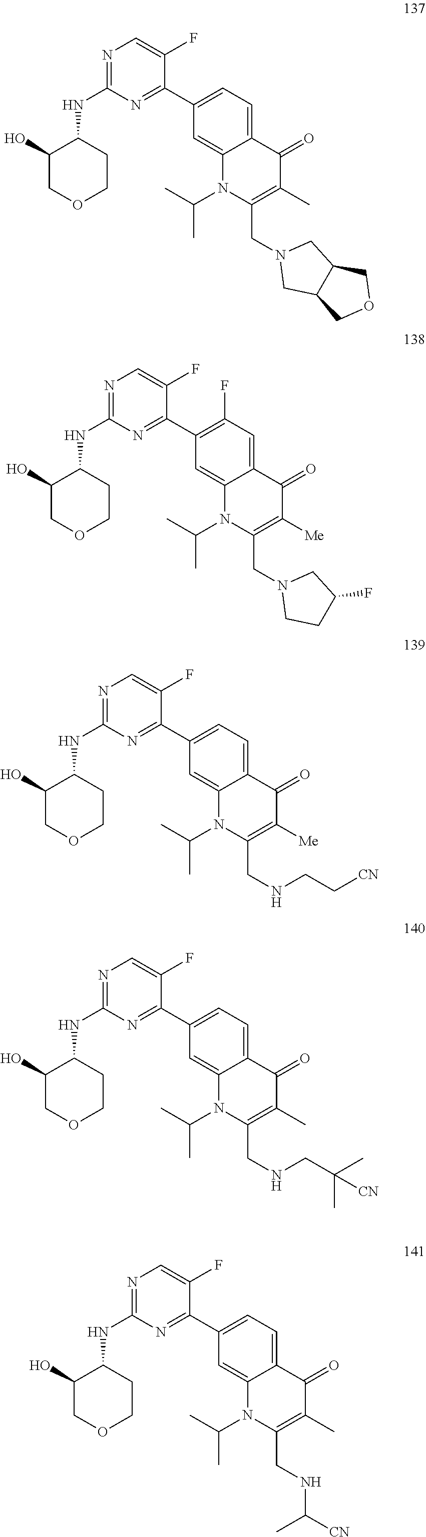 [SMILES: Cc1c(CN2C[C@H]3COC[C@H]3C2)n(C(C)C)c2cc(-c3nc(N[C@@H]4CCOC[C@H]4O)ncc3F)ccc2c1=O ; Cc1c(CN2CC[C@@H](F)C2)n(C(C)C)c2cc(-c3nc(N[C@@H]4CCOC[C@H]4O)ncc3F)c(F)cc2c1=O ; Cc1c(CNCCC#N)n(C(C)C)c2cc(-c3nc(N[C@@H]4CCOC[C@H]4O)ncc3F)ccc2c1=O ; Cc1c(CNCC(C)(C)C#N)n(C(C)C)c2cc(-c3nc(N[C@@H]4CCOC[C@H]4O)ncc3F)ccc2c1=O ; Cc1c(CNC(C)C#N)n(C(C)C)c2cc(-c3nc(N[C@@H]4CCOC[C@H]4O)ncc3F)ccc2c1=O]

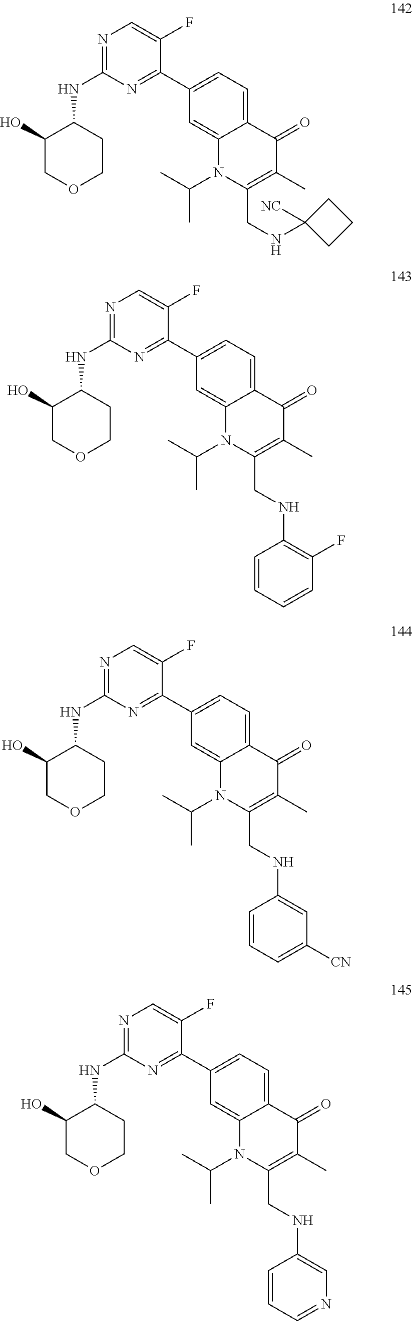 [SMILES: Cc1c(CNC2(C#N)CCC2)n(C(C)C)c2cc(-c3nc(N[C@@H]4CCOC[C@H]4O)ncc3F)ccc2c1=O ; Cc1c(CNc2ccccc2F)n(C(C)C)c2cc(-c3nc(N[C@@H]4CCOC[C@H]4O)ncc3F)ccc2c1=O ; Cc1c(CNc2cccc(C#N)c2)n(C(C)C)c2cc(-c3nc(N[C@@H]4CCOC[C@H]4O)ncc3F)ccc2c1=O ; Cc1c(CNc2cccnc2)n(C(C)C)c2cc(-c3nc(N[C@@H]4CCOC[C@H]4O)ncc3F)ccc2c1=O]

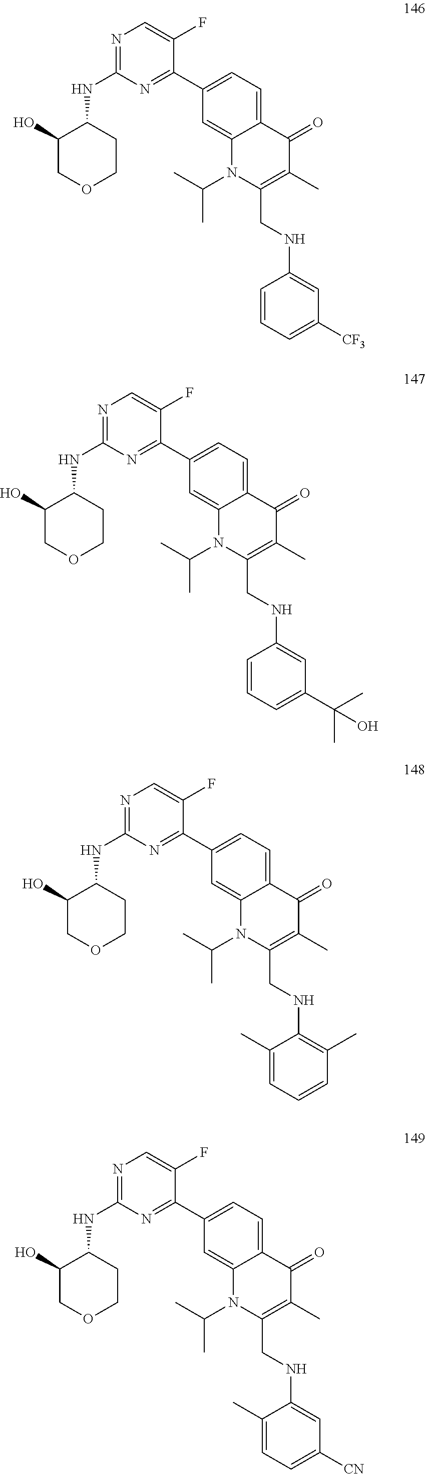 [SMILES: Cc1c(CNc2cccc(C(F)(F)F)c2)n(C(C)C)c2cc(-c3nc(N[C@@H]4CCOC[C@H]4O)ncc3F)ccc2c1=O ; Cc1c(CNc2cccc(C(C)(C)O)c2)n(C(C)C)c2cc(-c3nc(N[C@@H]4CCOC[C@H]4O)ncc3F)ccc2c1=O ; Cc1cccc(C)c1NCc1c(C)c(=O)c2ccc(-c3nc(N[C@@H]4CCOC[C@H]4O)ncc3F)cc2n1C(C)C ; Cc1ccc(C#N)cc1NCc1c(C)c(=O)c2ccc(-c3nc(N[C@@H]4CCOC[C@H]4O)ncc3F)cc2n1C(C)C]

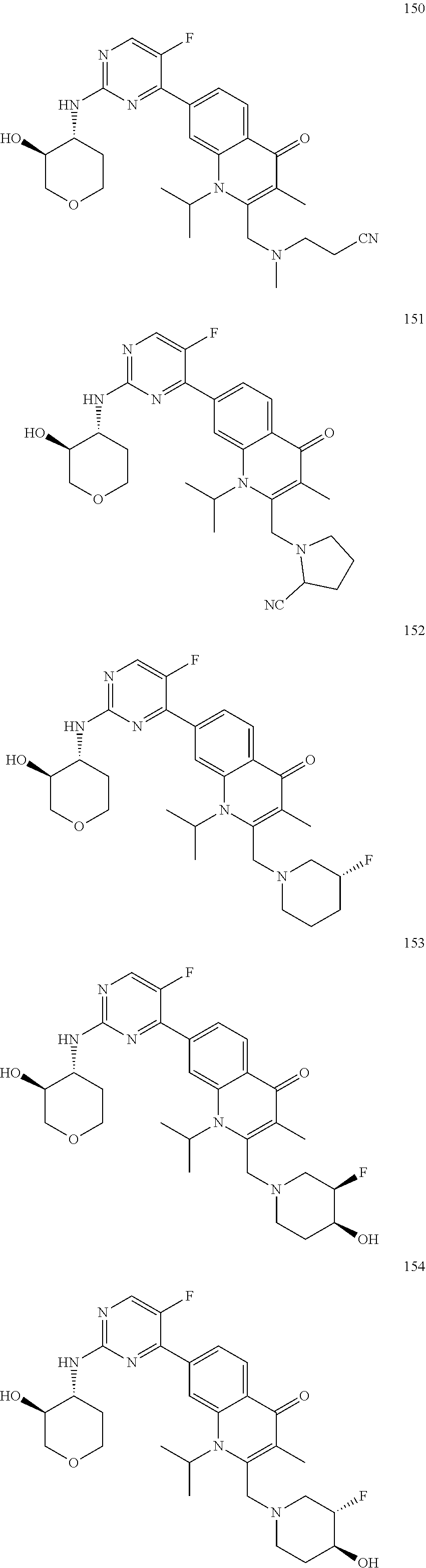 [SMILES: Cc1c(CN(C)CCC#N)n(C(C)C)c2cc(-c3nc(N[C@@H]4CCOC[C@H]4O)ncc3F)ccc2c1=O ; Cc1c(CN2CCCC2C#N)n(C(C)C)c2cc(-c3nc(N[C@@H]4CCOC[C@H]4O)ncc3F)ccc2c1=O ; Cc1c(CN2CCC[C@@H](F)C2)n(C(C)C)c2cc(-c3nc(N[C@@H]4CCOC[C@H]4O)ncc3F)ccc2c1=O ; Cc1c(CN2CC[C@H](O)[C@H](F)C2)n(C(C)C)c2cc(-c3nc(N[C@@H]4CCOC[C@H]4O)ncc3F)ccc2c1=O ; Cc1c(CN2CC[C@H](O)[C@@H](F)C2)n(C(C)C)c2cc(-c3nc(N[C@@H]4CCOC[C@H]4O)ncc3F)ccc2c1=O]

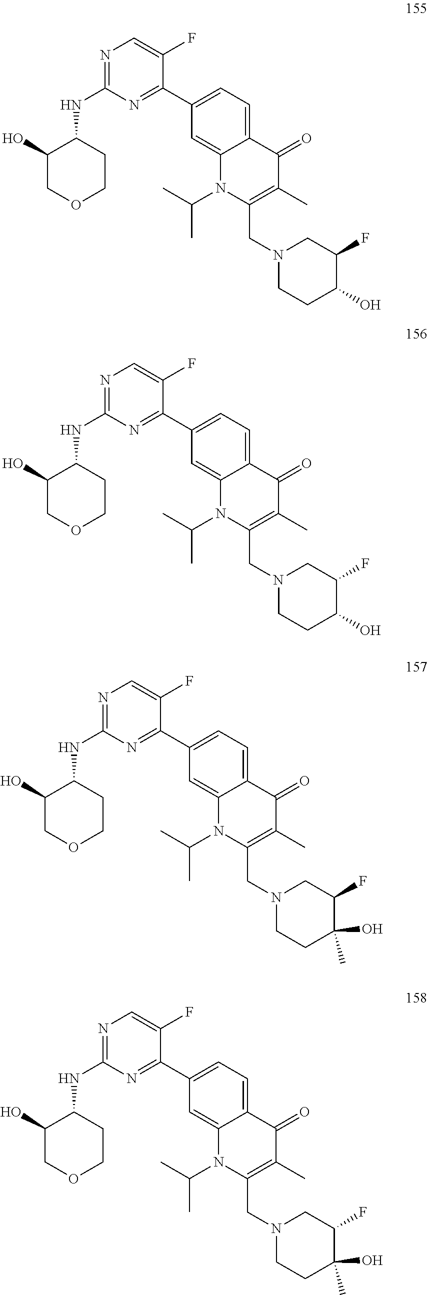 [SMILES: Cc1c(CN2CC[C@@H](O)[C@H](F)C2)n(C(C)C)c2cc(-c3nc(N[C@@H]4CCOC[C@H]4O)ncc3F)ccc2c1=O ; Cc1c(CN2CC[C@@H](O)[C@@H](F)C2)n(C(C)C)c2cc(-c3nc(N[C@@H]4CCOC[C@H]4O)ncc3F)ccc2c1=O ; Cc1c(CN2CC[C@](C)(O)[C@H](F)C2)n(C(C)C)c2cc(-c3nc(N[C@@H]4CCOC[C@H]4O)ncc3F)ccc2c1=O ; Cc1c(CN2CC[C@](C)(O)[C@@H](F)C2)n(C(C)C)c2cc(-c3nc(N[C@@H]4CCOC[C@H]4O)ncc3F)ccc2c1=O]

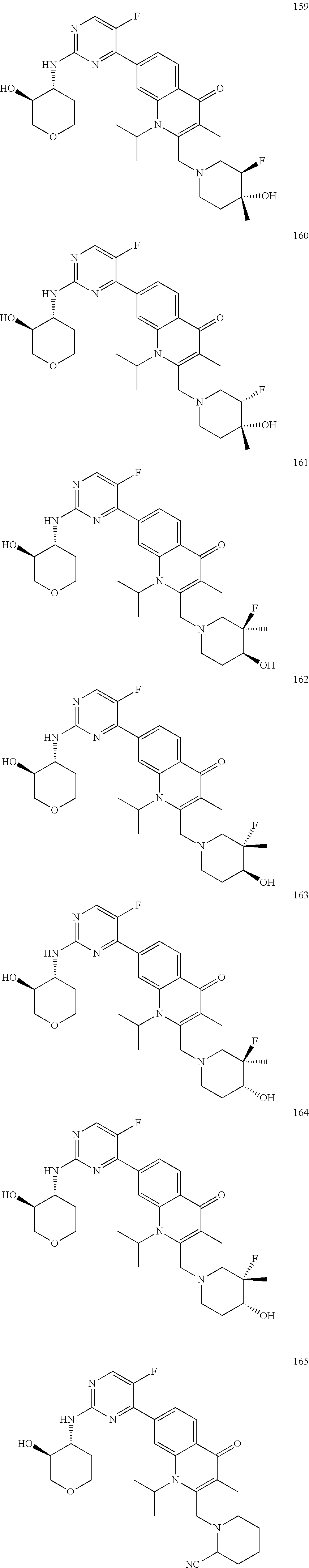

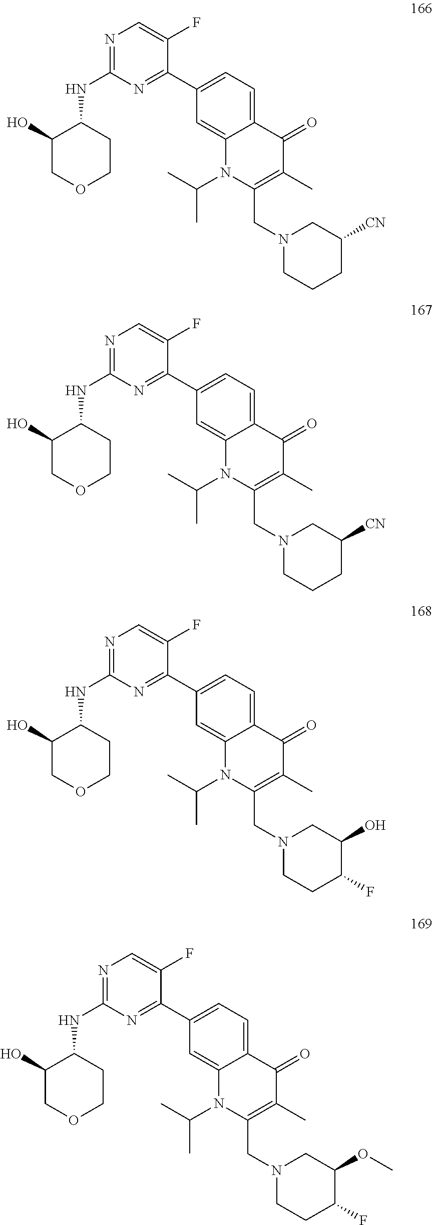 [SMILES: Cc1c(CN2CCC[C@@H](C#N)C2)n(C(C)C)c2cc(-c3nc(N[C@@H]4CCOC[C@H]4O)ncc3F)ccc2c1=O ; Cc1c(CN2CCC[C@H](C#N)C2)n(C(C)C)c2cc(-c3nc(N[C@@H]4CCOC[C@H]4O)ncc3F)ccc2c1=O ; Cc1c(CN2CC[C@@H](F)[C@H](O)C2)n(C(C)C)c2cc(-c3nc(N[C@@H]4CCOC[C@H]4O)ncc3F)ccc2c1=O ; CO[C@@H]1CN(Cc2c(C)c(=O)c3ccc(-c4nc(N[C@@H]5CCOC[C@H]5O)ncc4F)cc3n2C(C)C)CC[C@H]1F]

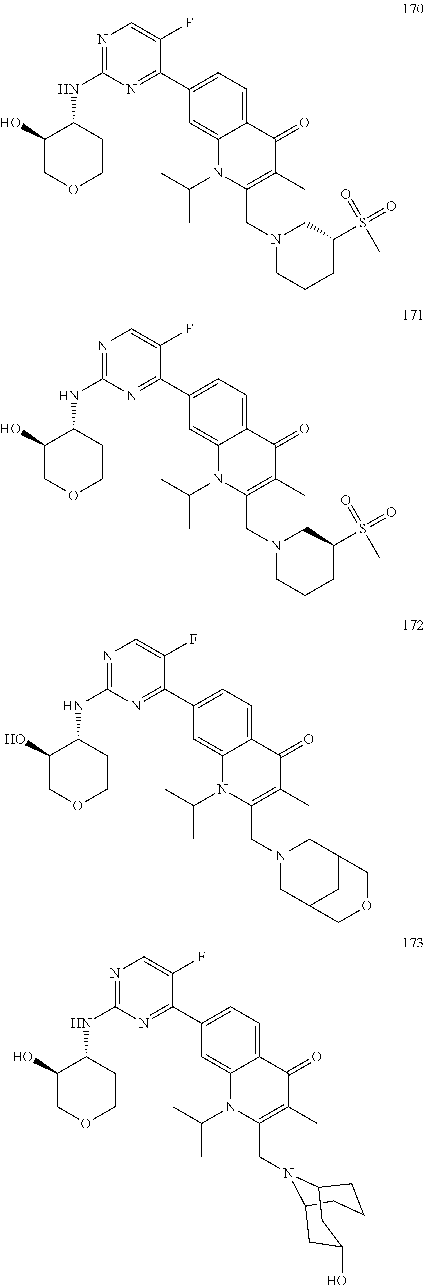 [SMILES: Cc1c(CN2CCC[C@@H](S(C)(=O)=O)C2)n(C(C)C)c2cc(-c3nc(N[C@@H]4CCOC[C@H]4O)ncc3F)ccc2c1=O ; Cc1c(CN2CCC[C@H](S(C)(=O)=O)C2)n(C(C)C)c2cc(-c3nc(N[C@@H]4CCOC[C@H]4O)ncc3F)ccc2c1=O ; Cc1c(CN2CC3COCC(C3)C2)n(C(C)C)c2cc(-c3nc(N[C@@H]4CCOC[C@H]4O)ncc3F)ccc2c1=O ; Cc1c(CN2C3CCCC2CC(O)C3)n(C(C)C)c2cc(-c3nc(N[C@@H]4CCOC[C@H]4O)ncc3F)ccc2c1=O]

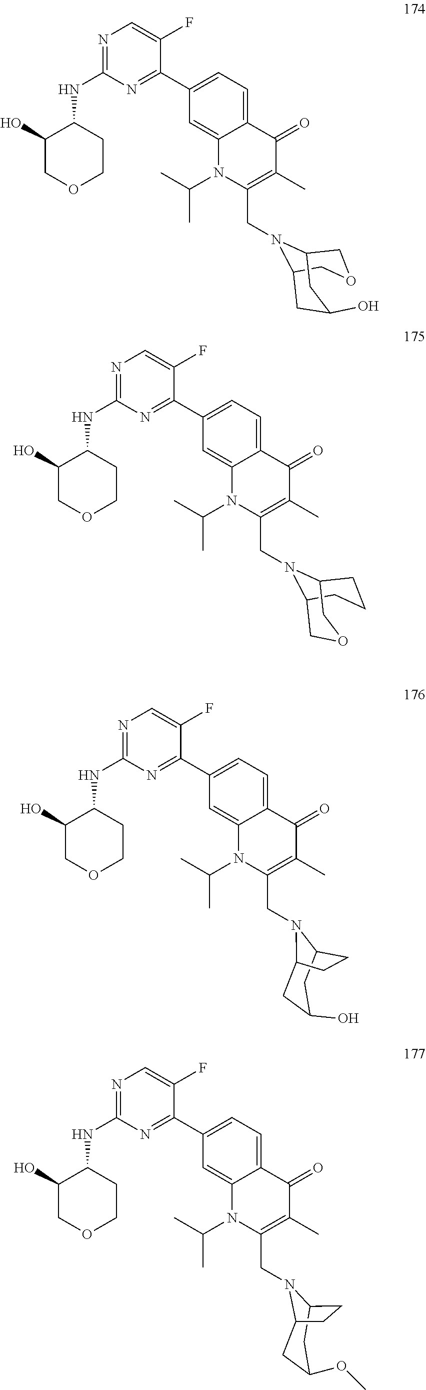 [SMILES: Cc1c(CN2C3COCC2CC(O)C3)n(C(C)C)c2cc(-c3nc(N[C@@H]4CCOC[C@H]4O)ncc3F)ccc2c1=O ; Cc1c(CN2C3CCCC2COC3)n(C(C)C)c2cc(-c3nc(N[C@@H]4CCOC[C@H]4O)ncc3F)ccc2c1=O ; Cc1c(CN2C3CCC2CC(O)C3)n(C(C)C)c2cc(-c3nc(N[C@@H]4CCOC[C@H]4O)ncc3F)ccc2c1=O ; COC1CC2CCC(C1)N2Cc1c(C)c(=O)c2ccc(-c3nc(N[C@@H]4CCOC[C@H]4O)ncc3F)cc2n1C(C)C]

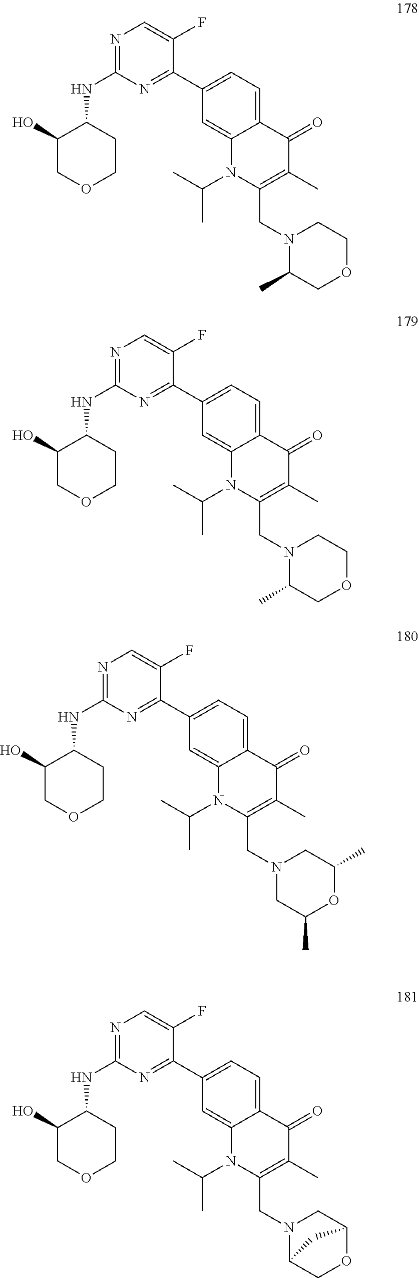 [SMILES: Cc1c(CN2CCOC[C@H]2C)n(C(C)C)c2cc(-c3nc(N[C@@H]4CCOC[C@H]4O)ncc3F)ccc2c1=O ; Cc1c(CN2CCOC[C@@H]2C)n(C(C)C)c2cc(-c3nc(N[C@@H]4CCOC[C@H]4O)ncc3F)ccc2c1=O ; Cc1c(CN2C[C@H](C)O[C@@H](C)C2)n(C(C)C)c2cc(-c3nc(N[C@@H]4CCOC[C@H]4O)ncc3F)ccc2c1=O ; Cc1c(CN2C[C@@H]3C[C@H]2CO3)n(C(C)C)c2cc(-c3nc(N[C@@H]4CCOC[C@H]4O)ncc3F)ccc2c1=O]

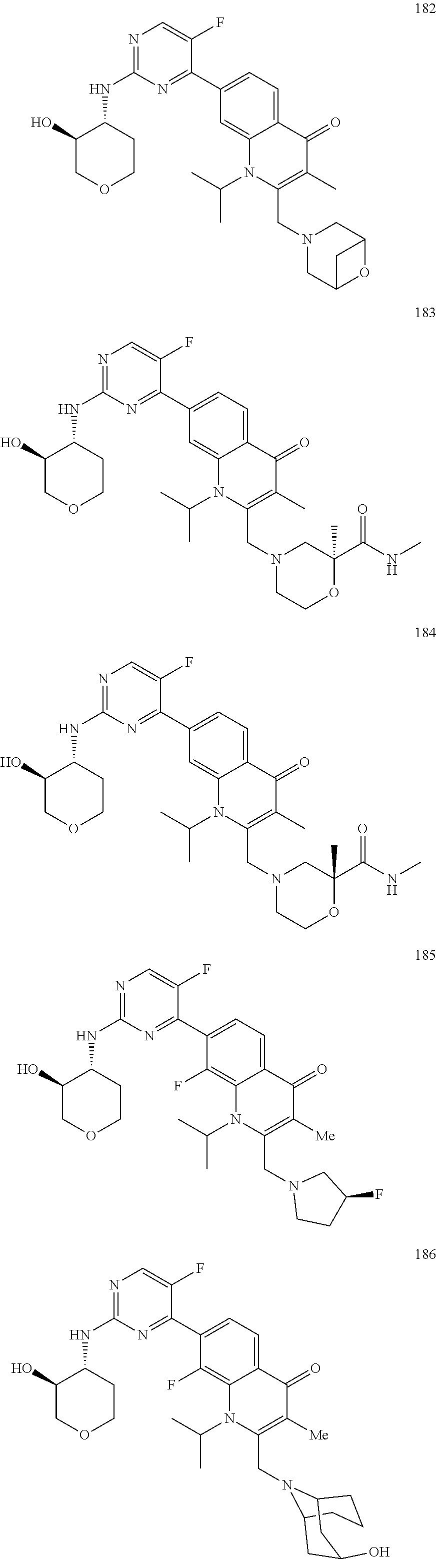 [SMILES: Cc1c(CN2CC3CC(C2)O3)n(C(C)C)c2cc(-c3nc(N[C@@H]4CCOC[C@H]4O)ncc3F)ccc2c1=O ; CNC(=O)[C@]1(C)CN(Cc2c(C)c(=O)c3ccc(-c4nc(N[C@@H]5CCOC[C@H]5O)ncc4F)cc3n2C(C)C)CCO1 ; CNC(=O)[C@@]1(C)CN(Cc2c(C)c(=O)c3ccc(-c4nc(N[C@@H]5CCOC[C@H]5O)ncc4F)cc3n2C(C)C)CCO1 ; Cc1c(CN2CC[C@H](F)C2)n(C(C)C)c2c(F)c(-c3nc(N[C@@H]4CCOC[C@H]4O)ncc3F)ccc2c1=O ; Cc1c(CN2C3CCCC2CC(O)C3)n(C(C)C)c2c(F)c(-c3nc(N[C@@H]4CCOC[C@H]4O)ncc3F)ccc2c1=O]

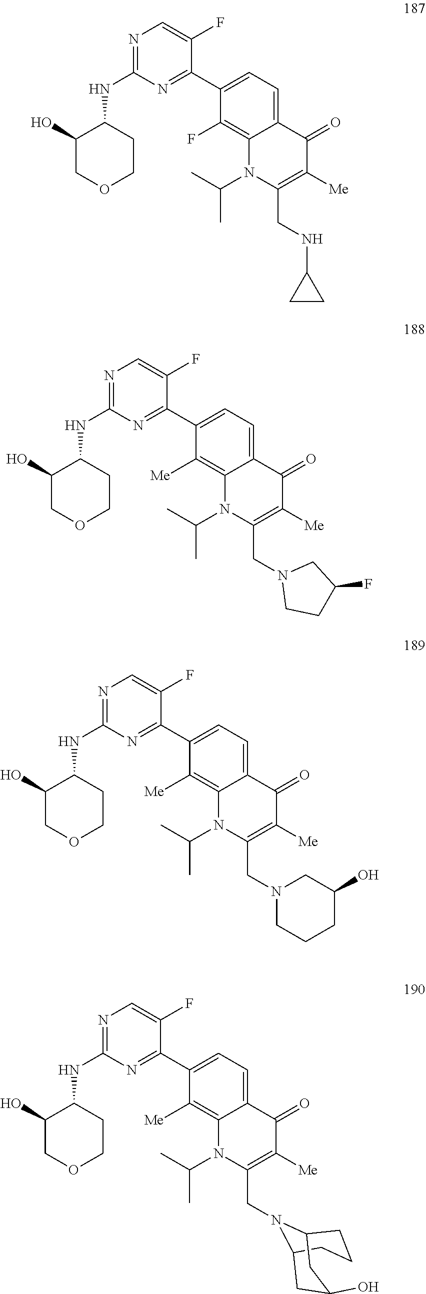 [SMILES: Cc1c(CNC2CC2)n(C(C)C)c2c(F)c(-c3nc(N[C@@H]4CCOC[C@H]4O)ncc3F)ccc2c1=O ; Cc1c(CN2CC[C@H](F)C2)n(C(C)C)c2c(C)c(-c3nc(N[C@@H]4CCOC[C@H]4O)ncc3F)ccc2c1=O ; Cc1c(CN2CCC[C@H](O)C2)n(C(C)C)c2c(C)c(-c3nc(N[C@@H]4CCOC[C@H]4O)ncc3F)ccc2c1=O ; Cc1c(CN2C3CCCC2CC(O)C3)n(C(C)C)c2c(C)c(-c3nc(N[C@@H]4CCOC[C@H]4O)ncc3F)ccc2c1=O]

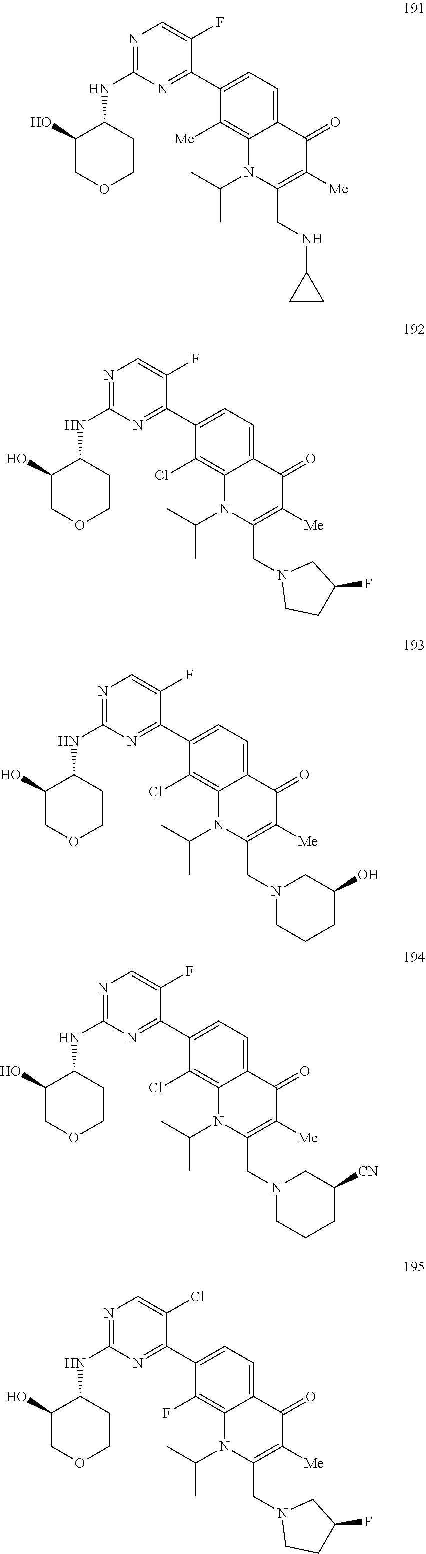 [SMILES: Cc1c(CNC2CC2)n(C(C)C)c2c(C)c(-c3nc(N[C@@H]4CCOC[C@H]4O)ncc3F)ccc2c1=O ; Cc1c(CN2CC[C@H](F)C2)n(C(C)C)c2c(Cl)c(-c3nc(N[C@@H]4CCOC[C@H]4O)ncc3F)ccc2c1=O ; Cc1c(CN2CCC[C@H](O)C2)n(C(C)C)c2c(Cl)c(-c3nc(N[C@@H]4CCOC[C@H]4O)ncc3F)ccc2c1=O ; Cc1c(CN2CCC[C@H](C#N)C2)n(C(C)C)c2c(Cl)c(-c3nc(N[C@@H]4CCOC[C@H]4O)ncc3F)ccc2c1=O ; Cc1c(CN2CC[C@H](F)C2)n(C(C)C)c2c(F)c(-c3nc(N[C@@H]4CCOC[C@H]4O)ncc3Cl)ccc2c1=O]

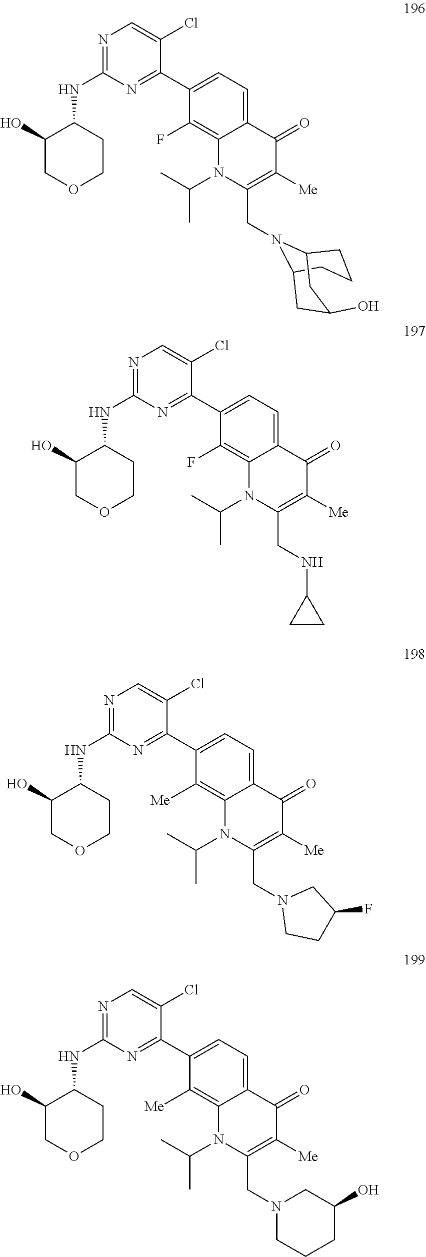 [SMILES: Cc1c(CN2C3CCCC2CC(O)C3)n(C(C)C)c2c(F)c(-c3nc(N[C@@H]4CCOC[C@H]4O)ncc3Cl)ccc2c1=O ; Cc1c(CNC2CC2)n(C(C)C)c2c(F)c(-c3nc(N[C@@H]4CCOC[C@H]4O)ncc3Cl)ccc2c1=O ; Cc1c(CN2CC[C@H](F)C2)n(C(C)C)c2c(C)c(-c3nc(N[C@@H]4CCOC[C@H]4O)ncc3Cl)ccc2c1=O ; Cc1c(CN2CCC[C@H](O)C2)n(C(C)C)c2c(C)c(-c3nc(N[C@@H]4CCOC[C@H]4O)ncc3Cl)ccc2c1=O]

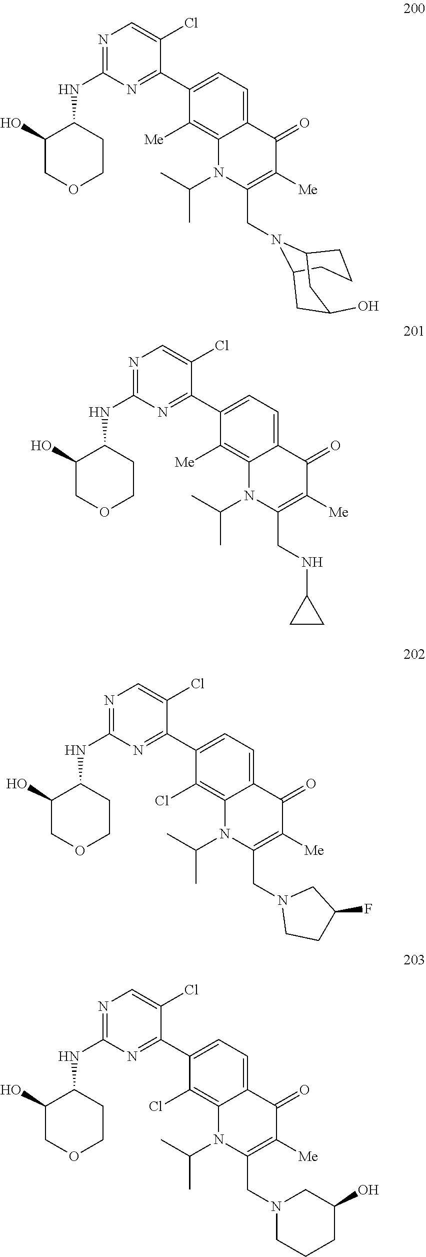 [SMILES: Cc1c(CN2C3CCCC2CC(O)C3)n(C(C)C)c2c(C)c(-c3nc(N[C@@H]4CCOC[C@H]4O)ncc3Cl)ccc2c1=O ; Cc1c(CNC2CC2)n(C(C)C)c2c(C)c(-c3nc(N[C@@H]4CCOC[C@H]4O)ncc3Cl)ccc2c1=O ; Cc1c(CN2CC[C@H](F)C2)n(C(C)C)c2c(Cl)c(-c3nc(N[C@@H]4CCOC[C@H]4O)ncc3Cl)ccc2c1=O ; Cc1c(CN2CCC[C@H](O)C2)n(C(C)C)c2c(Cl)c(-c3nc(N[C@@H]4CCOC[C@H]4O)ncc3Cl)ccc2c1=O]

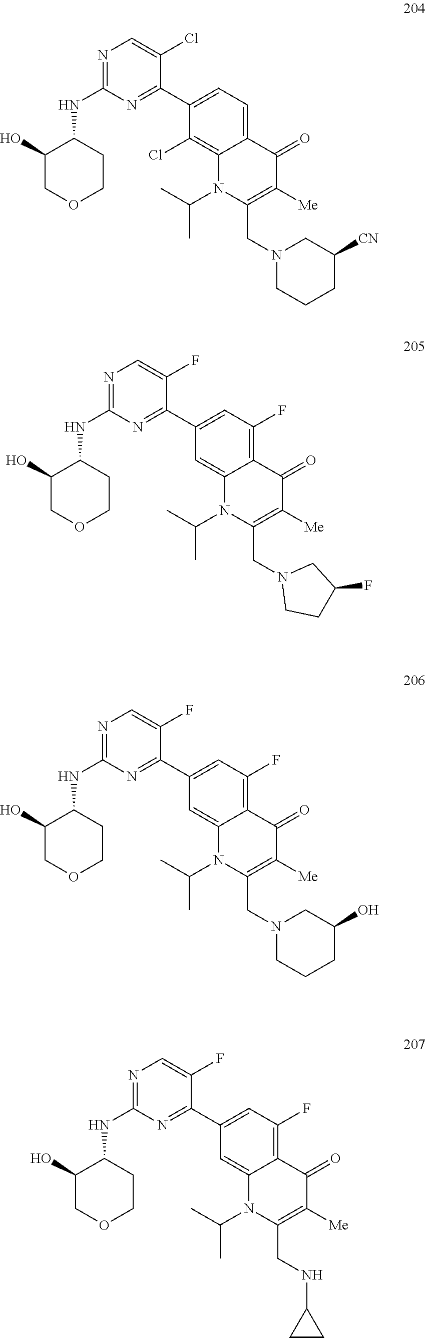 [SMILES: Cc1c(CN2CCC[C@H](C#N)C2)n(C(C)C)c2c(Cl)c(-c3nc(N[C@@H]4CCOC[C@H]4O)ncc3Cl)ccc2c1=O ; Cc1c(CN2CC[C@H](F)C2)n(C(C)C)c2cc(-c3nc(N[C@@H]4CCOC[C@H]4O)ncc3F)cc(F)c2c1=O ; Cc1c(CN2CCC[C@H](O)C2)n(C(C)C)c2cc(-c3nc(N[C@@H]4CCOC[C@H]4O)ncc3F)cc(F)c2c1=O ; Cc1c(CNC2CC2)n(C(C)C)c2cc(-c3nc(N[C@@H]4CCOC[C@H]4O)ncc3F)cc(F)c2c1=O]

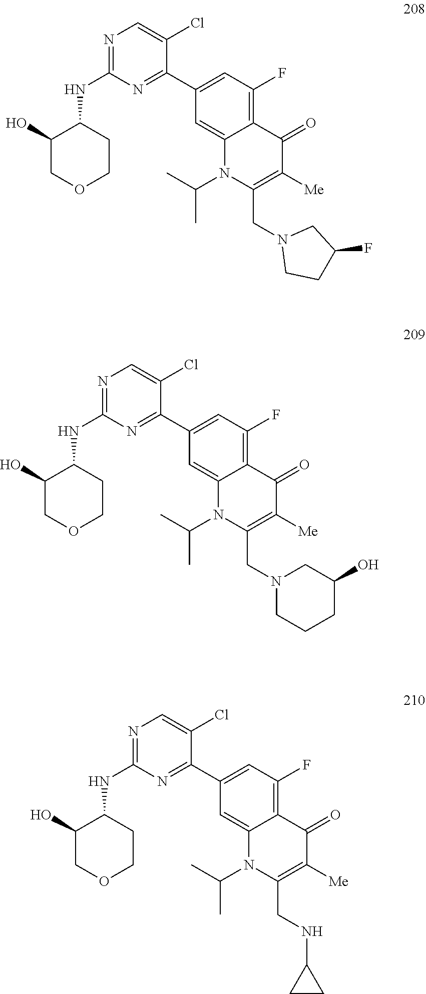 [SMILES: Cc1c(CN2CC[C@H](F)C2)n(C(C)C)c2cc(-c3nc(N[C@@H]4CCOC[C@H]4O)ncc3Cl)cc(F)c2c1=O ; Cc1c(CN2CCC[C@H](O)C2)n(C(C)C)c2cc(-c3nc(N[C@@H]4CCOC[C@H]4O)ncc3Cl)cc(F)c2c1=O ; Cc1c(CNC2CC2)n(C(C)C)c2cc(-c3nc(N[C@@H]4CCOC[C@H]4O)ncc3Cl)cc(F)c2c1=O]

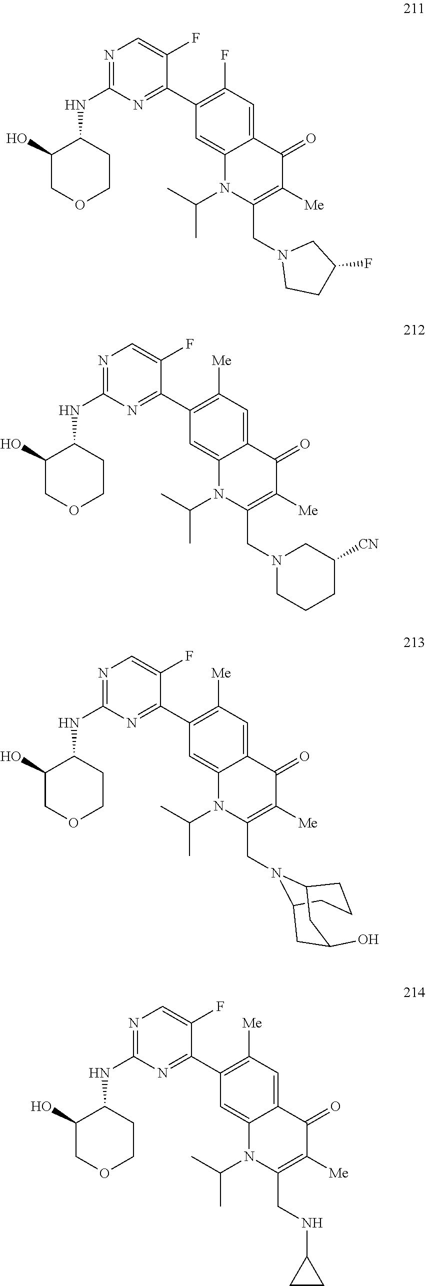 [SMILES: Cc1c(CN2CC[C@@H](F)C2)n(C(C)C)c2cc(-c3nc(N[C@@H]4CCOC[C@H]4O)ncc3F)c(F)cc2c1=O ; Cc1cc2c(=O)c(C)c(CN3CCC[C@@H](C#N)C3)n(C(C)C)c2cc1-c1nc(N[C@@H]2CCOC[C@H]2O)ncc1F ; Cc1cc2c(=O)c(C)c(CN3C4CCCC3CC(O)C4)n(C(C)C)c2cc1-c1nc(N[C@@H]2CCOC[C@H]2O)ncc1F ; Cc1cc2c(=O)c(C)c(CNC3CC3)n(C(C)C)c2cc1-c1nc(N[C@@H]2CCOC[C@H]2O)ncc1F]

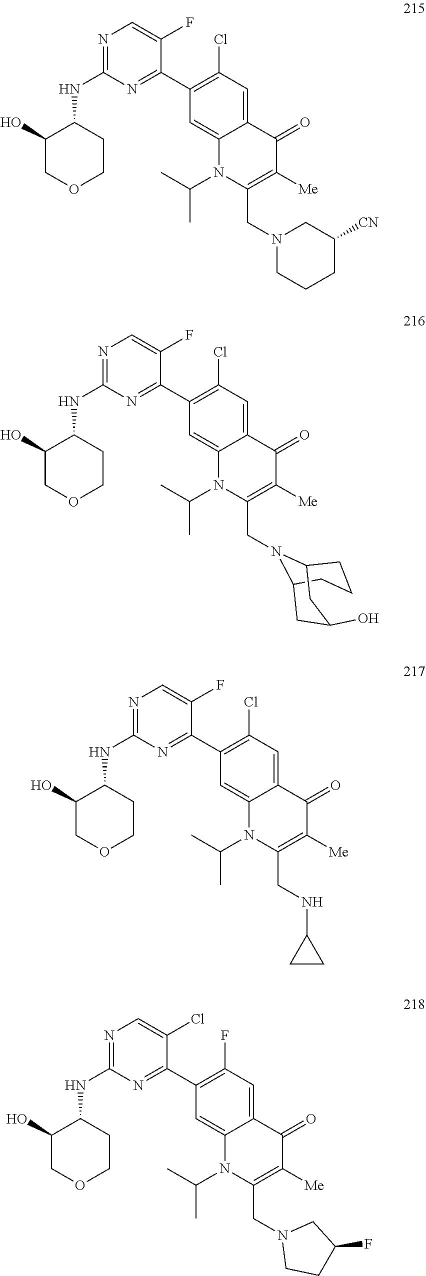 [SMILES: Cc1c(CN2CCC[C@@H](C#N)C2)n(C(C)C)c2cc(-c3nc(N[C@@H]4CCOC[C@H]4O)ncc3F)c(Cl)cc2c1=O ; Cc1c(CN2C3CCCC2CC(O)C3)n(C(C)C)c2cc(-c3nc(N[C@@H]4CCOC[C@H]4O)ncc3F)c(Cl)cc2c1=O ; Cc1c(CNC2CC2)n(C(C)C)c2cc(-c3nc(N[C@@H]4CCOC[C@H]4O)ncc3F)c(Cl)cc2c1=O ; Cc1c(CN2CC[C@H](F)C2)n(C(C)C)c2cc(-c3nc(N[C@@H]4CCOC[C@H]4O)ncc3Cl)c(F)cc2c1=O]

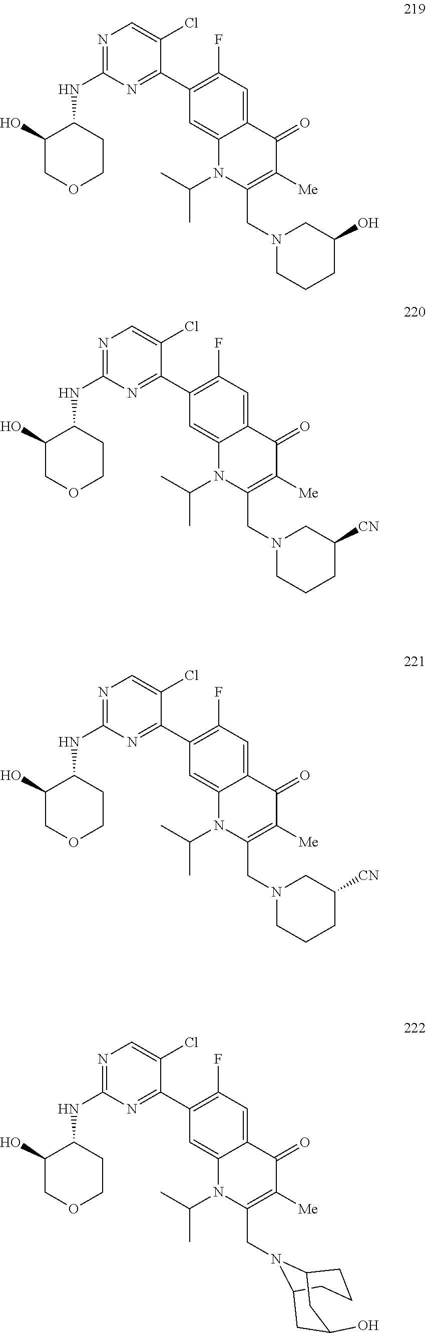 [SMILES: Cc1c(CN2CCC[C@H](O)C2)n(C(C)C)c2cc(-c3nc(N[C@@H]4CCOC[C@H]4O)ncc3Cl)c(F)cc2c1=O ; Cc1c(CN2CCC[C@H](C#N)C2)n(C(C)C)c2cc(-c3nc(N[C@@H]4CCOC[C@H]4O)ncc3Cl)c(F)cc2c1=O ; Cc1c(CN2CCC[C@@H](C#N)C2)n(C(C)C)c2cc(-c3nc(N[C@@H]4CCOC[C@H]4O)ncc3Cl)c(F)cc2c1=O ; Cc1c(CN2C3CCCC2CC(O)C3)n(C(C)C)c2cc(-c3nc(N[C@@H]4CCOC[C@H]4O)ncc3Cl)c(F)cc2c1=O]

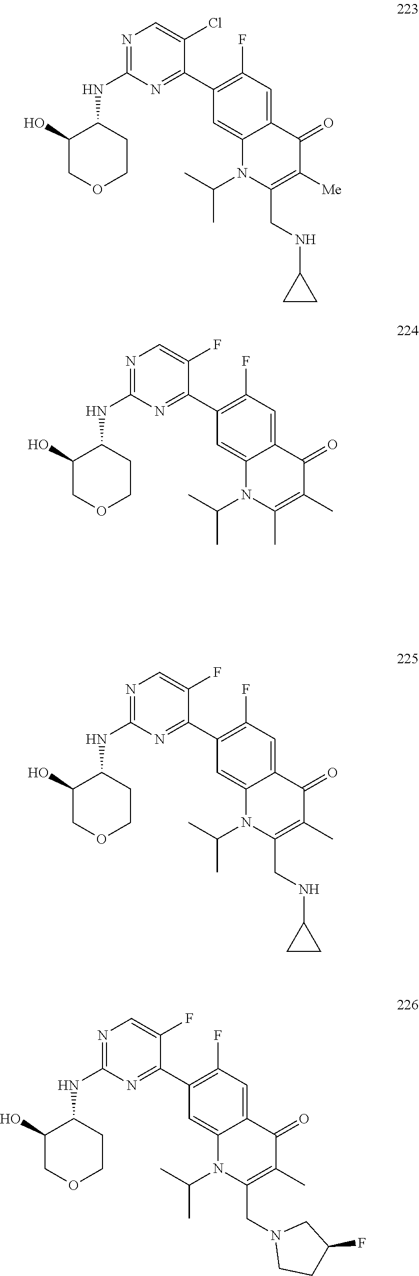 [SMILES: Cc1c(CNC2CC2)n(C(C)C)c2cc(-c3nc(N[C@@H]4CCOC[C@H]4O)ncc3Cl)c(F)cc2c1=O ; Cc1c(C)n(C(C)C)c2cc(-c3nc(N[C@@H]4CCOC[C@H]4O)ncc3F)c(F)cc2c1=O ; Cc1c(CNC2CC2)n(C(C)C)c2cc(-c3nc(N[C@@H]4CCOC[C@H]4O)ncc3F)c(F)cc2c1=O ; Cc1c(CN2CC[C@H](F)C2)n(C(C)C)c2cc(-c3nc(N[C@@H]4CCOC[C@H]4O)ncc3F)c(F)cc2c1=O]

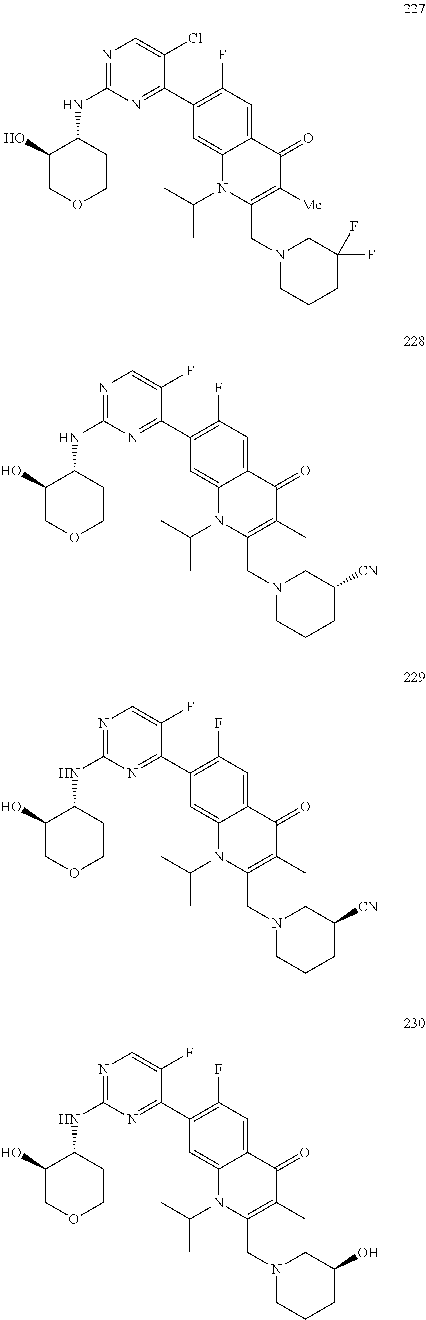 [SMILES: Cc1c(CN2CCCC(F)(F)C2)n(C(C)C)c2cc(-c3nc(N[C@@H]4CCOC[C@H]4O)ncc3Cl)c(F)cc2c1=O ; Cc1c(CN2CCC[C@@H](C#N)C2)n(C(C)C)c2cc(-c3nc(N[C@@H]4CCOC[C@H]4O)ncc3F)c(F)cc2c1=O ; Cc1c(CN2CCC[C@H](C#N)C2)n(C(C)C)c2cc(-c3nc(N[C@@H]4CCOC[C@H]4O)ncc3F)c(F)cc2c1=O ; Cc1c(CN2CCC[C@H](O)C2)n(C(C)C)c2cc(-c3nc(N[C@@H]4CCOC[C@H]4O)ncc3F)c(F)cc2c1=O]

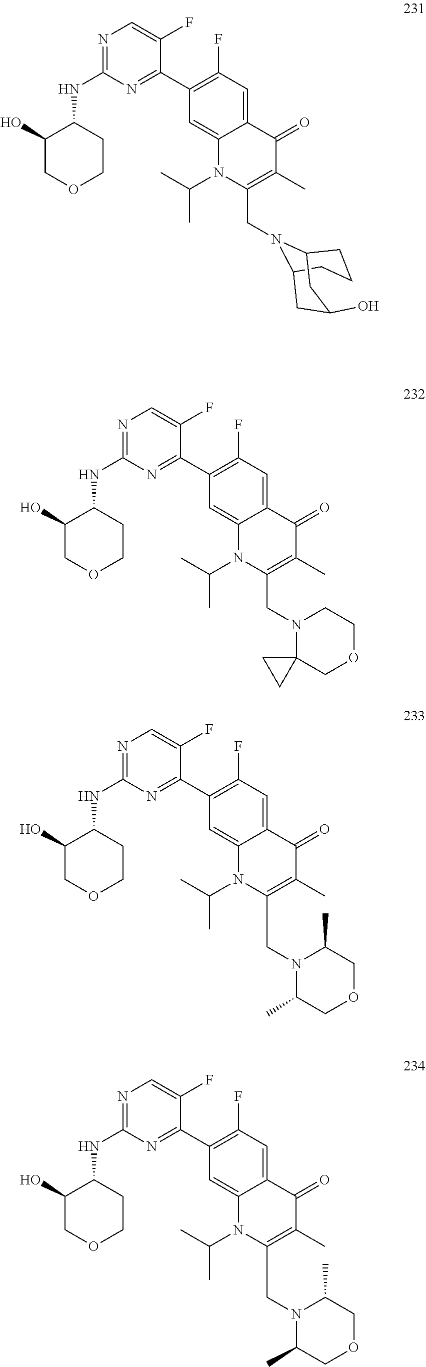 [SMILES: Cc1c(CN2C3CCCC2CC(O)C3)n(C(C)C)c2cc(-c3nc(N[C@@H]4CCOC[C@H]4O)ncc3F)c(F)cc2c1=O ; Cc1c(CN2CCOCC23CC3)n(C(C)C)c2cc(-c3nc(N[C@@H]4CCOC[C@H]4O)ncc3F)c(F)cc2c1=O ; Cc1c(CN2[C@@H](C)COC[C@@H]2C)n(C(C)C)c2cc(-c3nc(N[C@@H]4CCOC[C@H]4O)ncc3F)c(F)cc2c1=O ; Cc1c(CN2[C@H](C)COC[C@H]2C)n(C(C)C)c2cc(-c3nc(N[C@@H]4CCOC[C@H]4O)ncc3F)c(F)cc2c1=O]

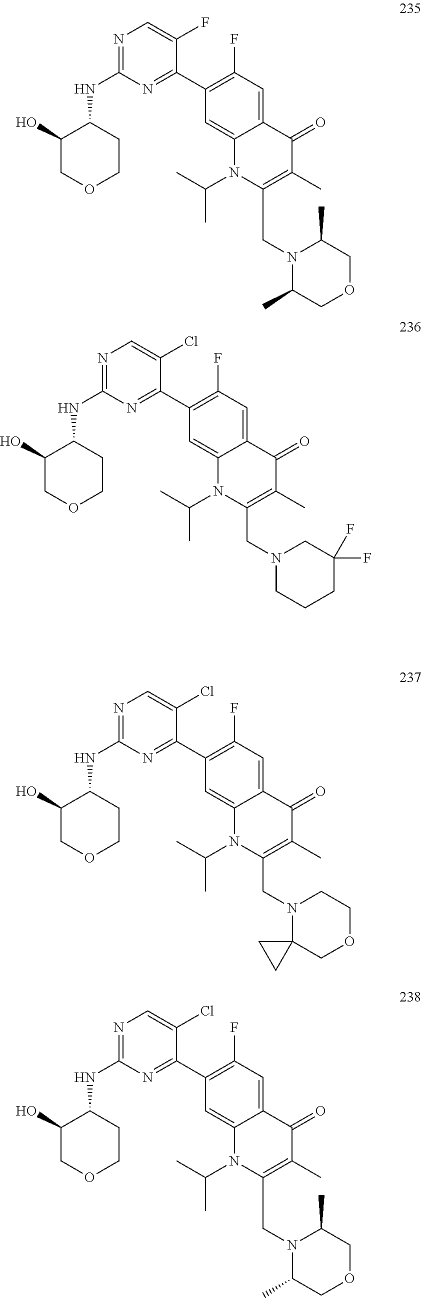 [SMILES: Cc1c(CN2[C@H](C)COC[C@@H]2C)n(C(C)C)c2cc(-c3nc(N[C@@H]4CCOC[C@H]4O)ncc3F)c(F)cc2c1=O ; Cc1c(CN2CCCC(F)(F)C2)n(C(C)C)c2cc(-c3nc(N[C@@H]4CCOC[C@H]4O)ncc3Cl)c(F)cc2c1=O ; Cc1c(CN2CCOCC23CC3)n(C(C)C)c2cc(-c3nc(N[C@@H]4CCOC[C@H]4O)ncc3Cl)c(F)cc2c1=O ; Cc1c(CN2[C@@H](C)COC[C@@H]2C)n(C(C)C)c2cc(-c3nc(N[C@@H]4CCOC[C@H]4O)ncc3Cl)c(F)cc2c1=O]

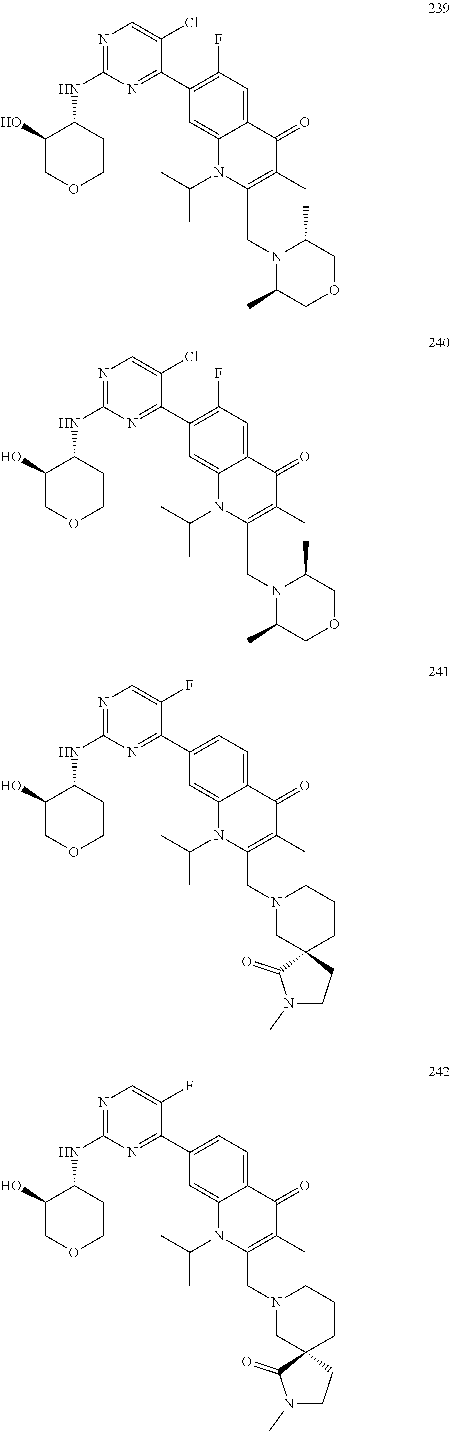 [SMILES: Cc1c(CN2[C@H](C)COC[C@H]2C)n(C(C)C)c2cc(-c3nc(N[C@@H]4CCOC[C@H]4O)ncc3Cl)c(F)cc2c1=O ; Cc1c(CN2[C@H](C)COC[C@@H]2C)n(C(C)C)c2cc(-c3nc(N[C@@H]4CCOC[C@H]4O)ncc3Cl)c(F)cc2c1=O ; Cc1c(CN2CCC[C@@]3(CCN(C)C3=O)C2)n(C(C)C)c2cc(-c3nc(N[C@@H]4CCOC[C@H]4O)ncc3F)ccc2c1=O ; Cc1c(CN2CCC[C@]3(CCN(C)C3=O)C2)n(C(C)C)c2cc(-c3nc(N[C@@H]4CCOC[C@H]4O)ncc3F)ccc2c1=O]

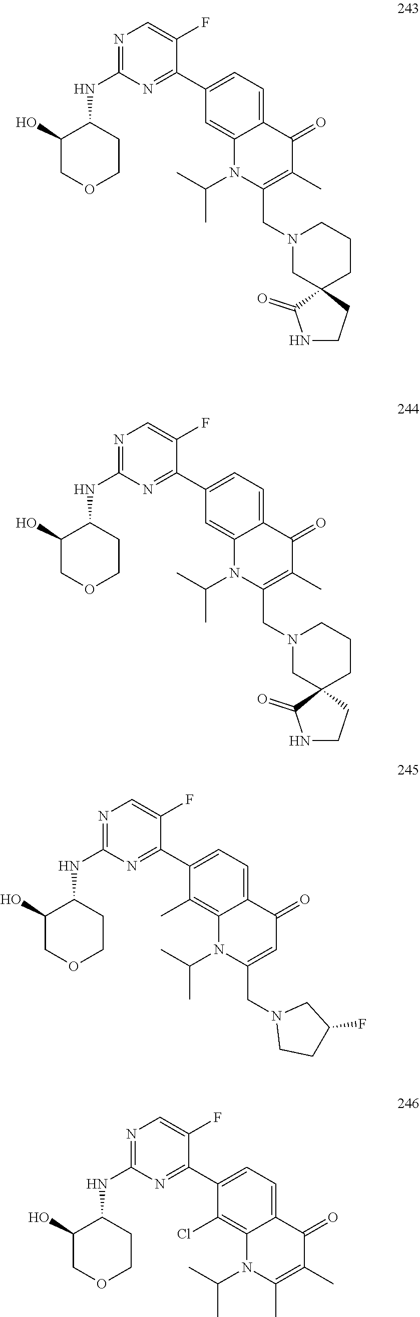 [SMILES: Cc1c(CN2CCC[C@@]3(CCNC3=O)C2)n(C(C)C)c2cc(-c3nc(N[C@@H]4CCOC[C@H]4O)ncc3F)ccc2c1=O ; Cc1c(CN2CCC[C@]3(CCNC3=O)C2)n(C(C)C)c2cc(-c3nc(N[C@@H]4CCOC[C@H]4O)ncc3F)ccc2c1=O ; Cc1c(-c2nc(N[C@@H]3CCOC[C@H]3O)ncc2F)ccc2c(=O)cc(CN3CC[C@@H](F)C3)n(C(C)C)c12 ; Cc1c(C)n(C(C)C)c2c(Cl)c(-c3nc(N[C@@H]4CCOC[C@H]4O)ncc3F)ccc2c1=O]

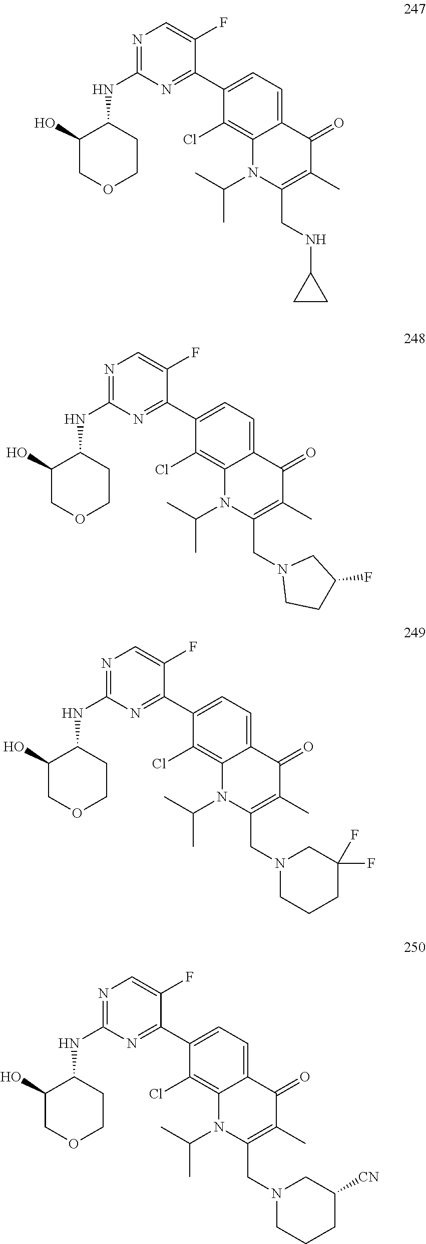 [SMILES: Cc1c(CNC2CC2)n(C(C)C)c2c(Cl)c(-c3nc(N[C@@H]4CCOC[C@H]4O)ncc3F)ccc2c1=O ; Cc1c(CN2CC[C@@H](F)C2)n(C(C)C)c2c(Cl)c(-c3nc(N[C@@H]4CCOC[C@H]4O)ncc3F)ccc2c1=O ; Cc1c(CN2CCCC(F)(F)C2)n(C(C)C)c2c(Cl)c(-c3nc(N[C@@H]4CCOC[C@H]4O)ncc3F)ccc2c1=O ; Cc1c(CN2CCC[C@@H](C#N)C2)n(C(C)C)c2c(Cl)c(-c3nc(N[C@@H]4CCOC[C@H]4O)ncc3F)ccc2c1=O]

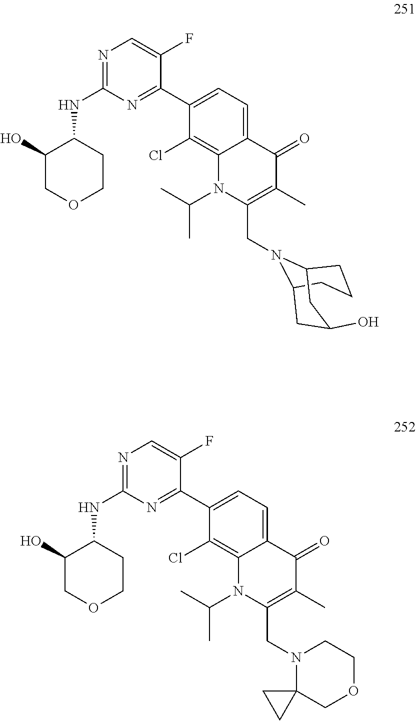 [SMILES: Cc1c(CN2C3CCCC2CC(O)C3)n(C(C)C)c2c(Cl)c(-c3nc(N[C@@H]4CCOC[C@H]4O)ncc3F)ccc2c1=O ; Cc1c(CN2CCOCC23CC3)n(C(C)C)c2c(Cl)c(-c3nc(N[C@@H]4CCOC[C@H]4O)ncc3F)ccc2c1=O]

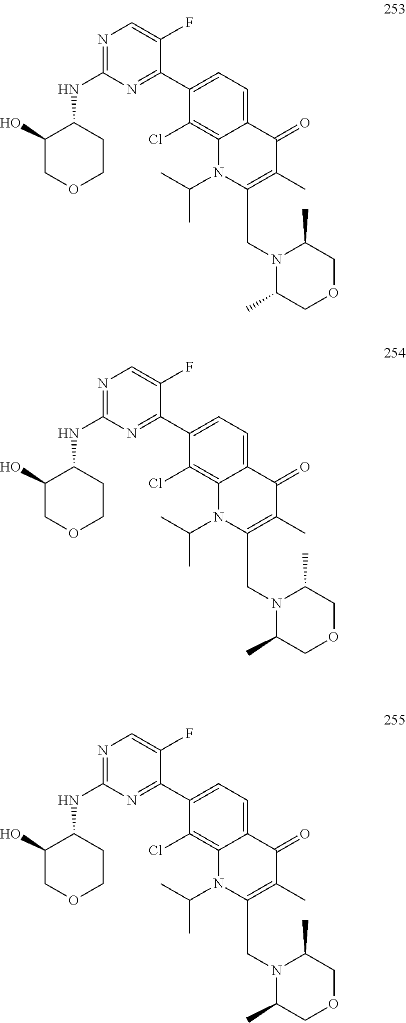 [SMILES: Cc1c(CN2[C@@H](C)COC[C@@H]2C)n(C(C)C)c2c(Cl)c(-c3nc(N[C@@H]4CCOC[C@H]4O)ncc3F)ccc2c1=O ; Cc1c(CN2[C@H](C)COC[C@H]2C)n(C(C)C)c2c(Cl)c(-c3nc(N[C@@H]4CCOC[C@H]4O)ncc3F)ccc2c1=O ; Cc1c(CN2[C@H](C)COC[C@@H]2C)n(C(C)C)c2c(Cl)c(-c3nc(N[C@@H]4CCOC[C@H]4O)ncc3F)ccc2c1=O]

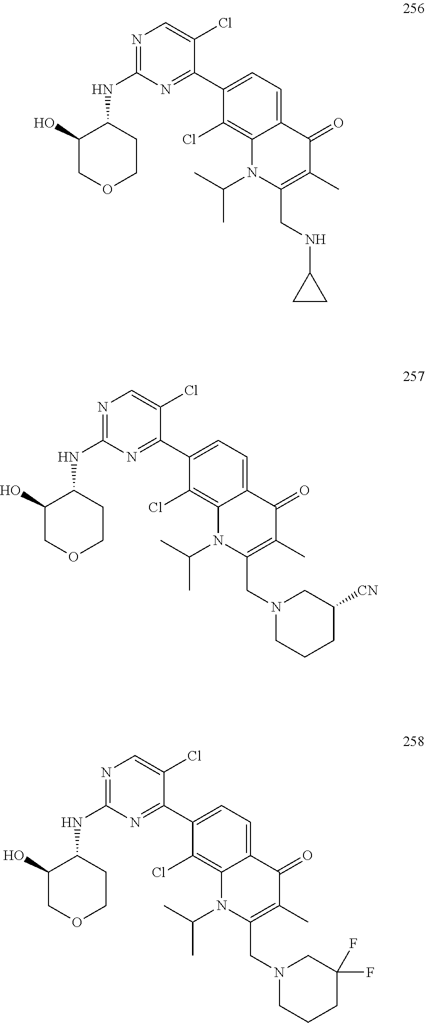 [SMILES: Cc1c(CNC2CC2)n(C(C)C)c2c(Cl)c(-c3nc(N[C@@H]4CCOC[C@H]4O)ncc3Cl)ccc2c1=O ; Cc1c(CN2CCC[C@@H](C#N)C2)n(C(C)C)c2c(Cl)c(-c3nc(N[C@@H]4CCOC[C@H]4O)ncc3Cl)ccc2c1=O ; Cc1c(CN2CCCC(F)(F)C2)n(C(C)C)c2c(Cl)c(-c3nc(N[C@@H]4CCOC[C@H]4O)ncc3Cl)ccc2c1=O]

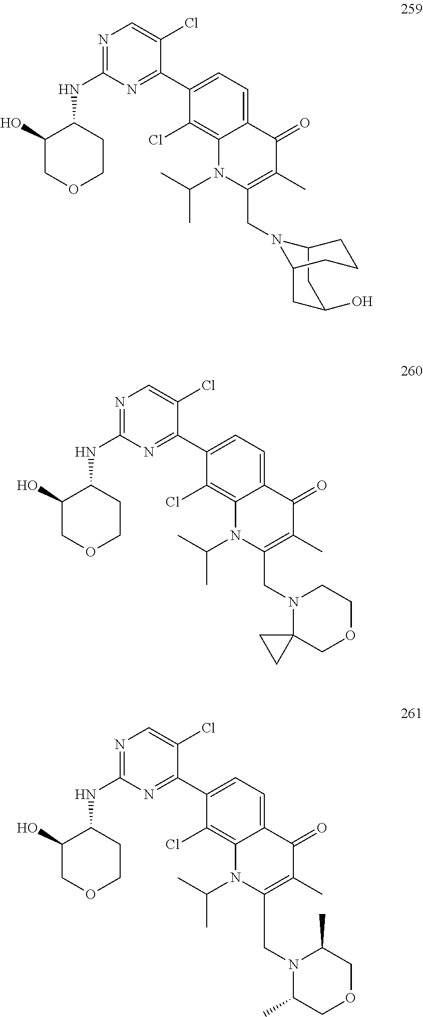 [SMILES: Cc1c(CN2C3CCCC2CC(O)C3)n(C(C)C)c2c(Cl)c(-c3nc(N[C@@H]4CCOC[C@H]4O)ncc3Cl)ccc2c1=O ; Cc1c(CN2CCOCC23CC3)n(C(C)C)c2c(Cl)c(-c3nc(N[C@@H]4CCOC[C@H]4O)ncc3Cl)ccc2c1=O ; Cc1c(CN2[C@@H](C)COC[C@@H]2C)n(C(C)C)c2c(Cl)c(-c3nc(N[C@@H]4CCOC[C@H]4O)ncc3Cl)ccc2c1=O]

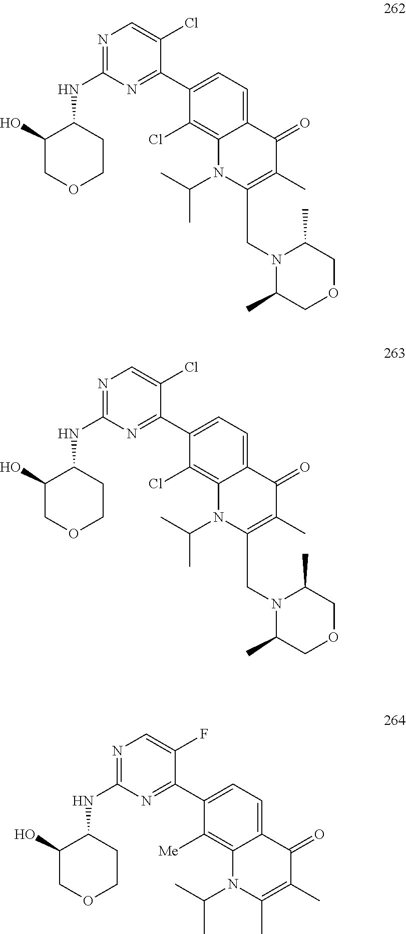 [SMILES: Cc1c(CN2[C@H](C)COC[C@H]2C)n(C(C)C)c2c(Cl)c(-c3nc(N[C@@H]4CCOC[C@H]4O)ncc3Cl)ccc2c1=O ; Cc1c(CN2[C@H](C)COC[C@@H]2C)n(C(C)C)c2c(Cl)c(-c3nc(N[C@@H]4CCOC[C@H]4O)ncc3Cl)ccc2c1=O ; Cc1c(C)n(C(C)C)c2c(C)c(-c3nc(N[C@@H]4CCOC[C@H]4O)ncc3F)ccc2c1=O]

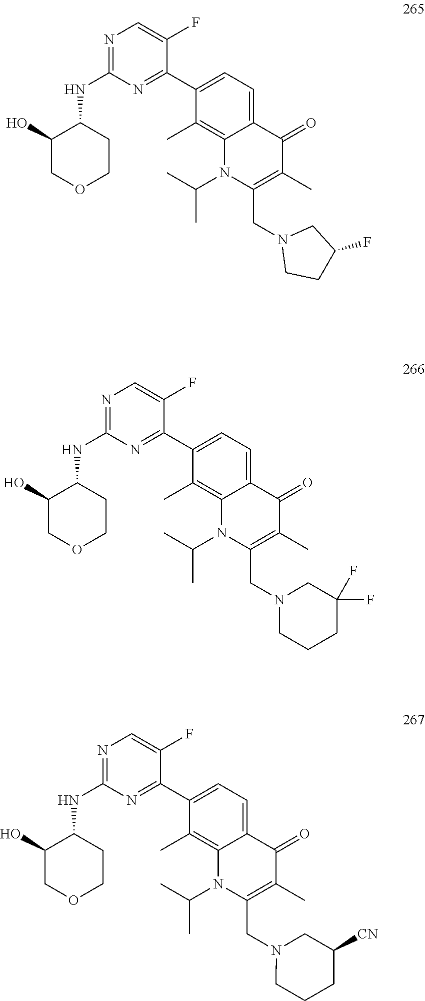 [SMILES: Cc1c(CN2CC[C@@H](F)C2)n(C(C)C)c2c(C)c(-c3nc(N[C@@H]4CCOC[C@H]4O)ncc3F)ccc2c1=O ; Cc1c(CN2CCCC(F)(F)C2)n(C(C)C)c2c(C)c(-c3nc(N[C@@H]4CCOC[C@H]4O)ncc3F)ccc2c1=O ; Cc1c(CN2CCC[C@H](C#N)C2)n(C(C)C)c2c(C)c(-c3nc(N[C@@H]4CCOC[C@H]4O)ncc3F)ccc2c1=O]

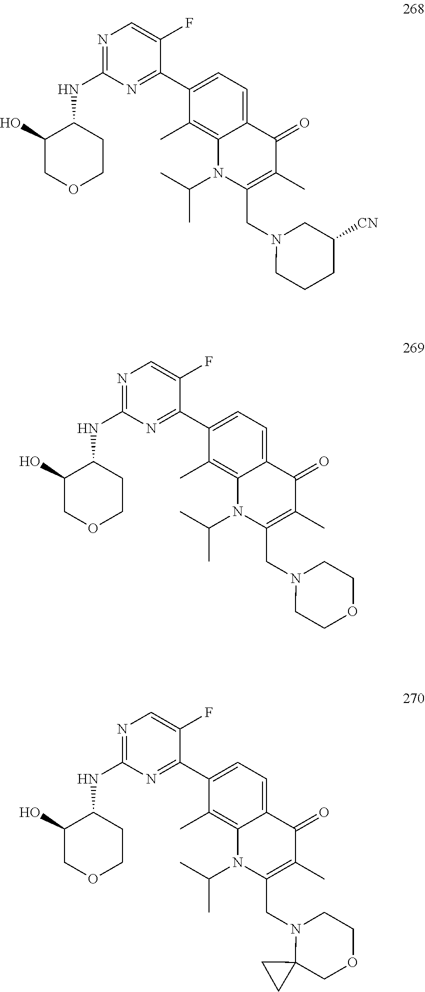 [SMILES: Cc1c(CN2CCC[C@@H](C#N)C2)n(C(C)C)c2c(C)c(-c3nc(N[C@@H]4CCOC[C@H]4O)ncc3F)ccc2c1=O ; Cc1c(CN2CCOCC2)n(C(C)C)c2c(C)c(-c3nc(N[C@@H]4CCOC[C@H]4O)ncc3F)ccc2c1=O ; Cc1c(CN2CCOCC23CC3)n(C(C)C)c2c(C)c(-c3nc(N[C@@H]4CCOC[C@H]4O)ncc3F)ccc2c1=O]

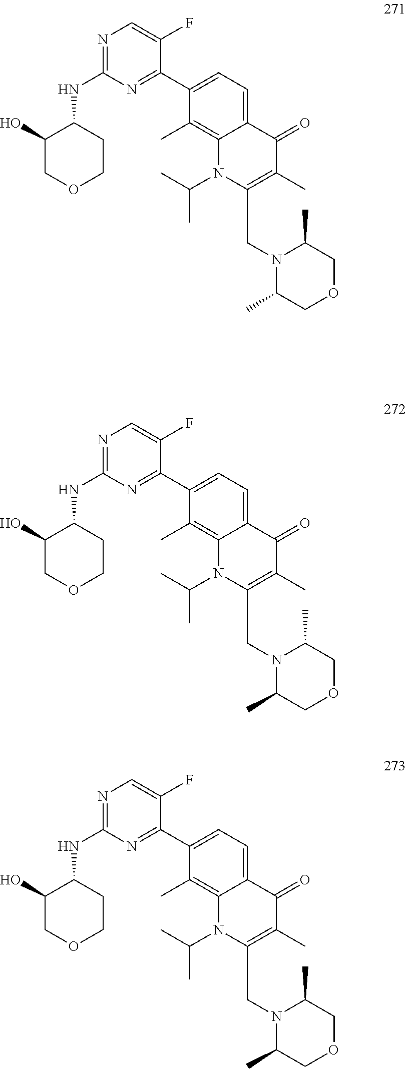 [SMILES: Cc1c(CN2[C@@H](C)COC[C@@H]2C)n(C(C)C)c2c(C)c(-c3nc(N[C@@H]4CCOC[C@H]4O)ncc3F)ccc2c1=O ; Cc1c(CN2[C@H](C)COC[C@H]2C)n(C(C)C)c2c(C)c(-c3nc(N[C@@H]4CCOC[C@H]4O)ncc3F)ccc2c1=O ; Cc1c(CN2[C@H](C)COC[C@@H]2C)n(C(C)C)c2c(C)c(-c3nc(N[C@@H]4CCOC[C@H]4O)ncc3F)ccc2c1=O]

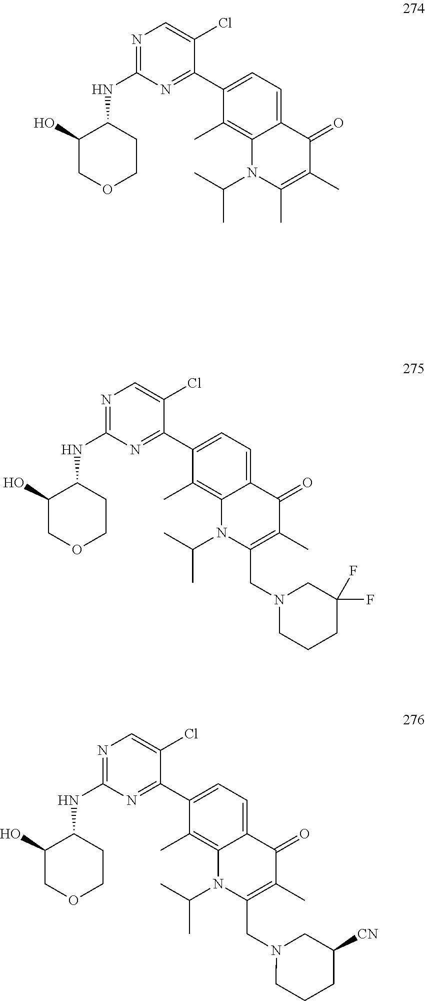 [SMILES: Cc1c(C)n(C(C)C)c2c(C)c(-c3nc(N[C@@H]4CCOC[C@H]4O)ncc3Cl)ccc2c1=O ; Cc1c(CN2CCCC(F)(F)C2)n(C(C)C)c2c(C)c(-c3nc(N[C@@H]4CCOC[C@H]4O)ncc3Cl)ccc2c1=O ; Cc1c(CN2CCC[C@H](C#N)C2)n(C(C)C)c2c(C)c(-c3nc(N[C@@H]4CCOC[C@H]4O)ncc3Cl)ccc2c1=O]

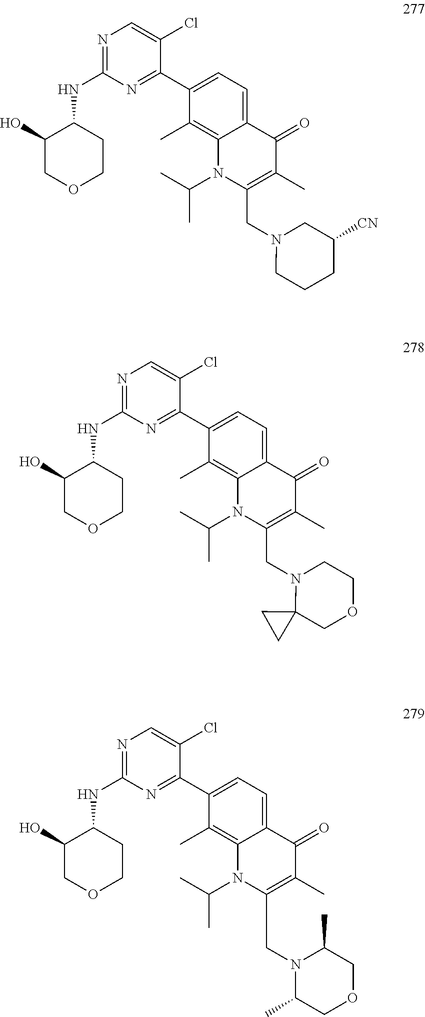 [SMILES: Cc1c(CN2CCC[C@@H](C#N)C2)n(C(C)C)c2c(C)c(-c3nc(N[C@@H]4CCOC[C@H]4O)ncc3Cl)ccc2c1=O ; Cc1c(CN2CCOCC23CC3)n(C(C)C)c2c(C)c(-c3nc(N[C@@H]4CCOC[C@H]4O)ncc3Cl)ccc2c1=O ; Cc1c(CN2[C@@H](C)COC[C@@H]2C)n(C(C)C)c2c(C)c(-c3nc(N[C@@H]4CCOC[C@H]4O)ncc3Cl)ccc2c1=O]

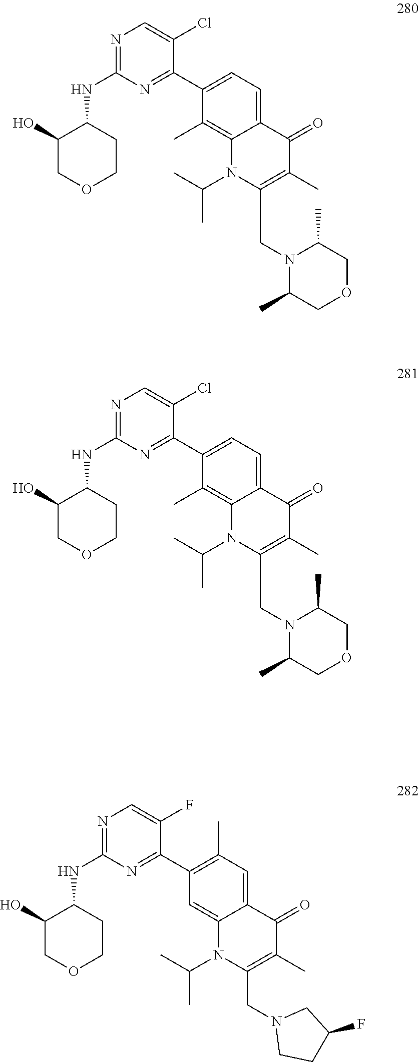 [SMILES: Cc1c(CN2[C@H](C)COC[C@H]2C)n(C(C)C)c2c(C)c(-c3nc(N[C@@H]4CCOC[C@H]4O)ncc3Cl)ccc2c1=O ; Cc1c(CN2[C@H](C)COC[C@@H]2C)n(C(C)C)c2c(C)c(-c3nc(N[C@@H]4CCOC[C@H]4O)ncc3Cl)ccc2c1=O ; Cc1cc2c(=O)c(C)c(CN3CC[C@H](F)C3)n(C(C)C)c2cc1-c1nc(N[C@@H]2CCOC[C@H]2O)ncc1F]

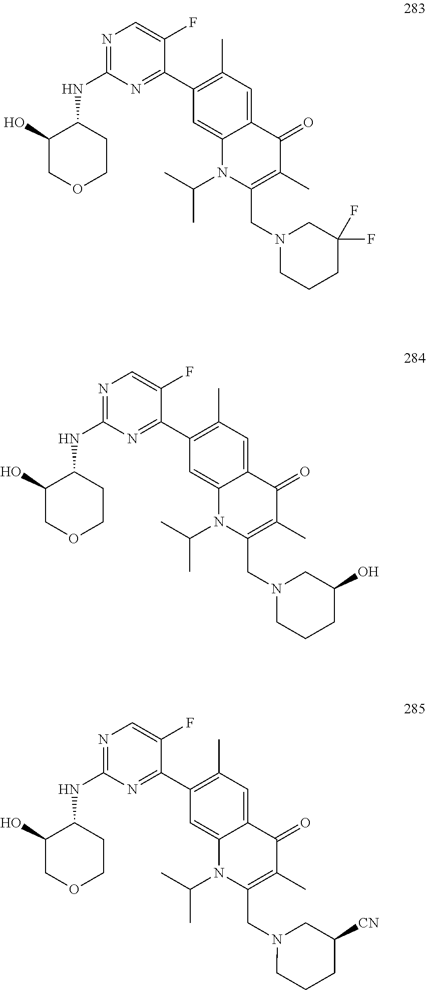 [SMILES: Cc1cc2c(=O)c(C)c(CN3CCCC(F)(F)C3)n(C(C)C)c2cc1-c1nc(N[C@@H]2CCOC[C@H]2O)ncc1F ; Cc1cc2c(=O)c(C)c(CN3CCC[C@H](O)C3)n(C(C)C)c2cc1-c1nc(N[C@@H]2CCOC[C@H]2O)ncc1F ; Cc1cc2c(=O)c(C)c(CN3CCC[C@H](C#N)C3)n(C(C)C)c2cc1-c1nc(N[C@@H]2CCOC[C@H]2O)ncc1F]

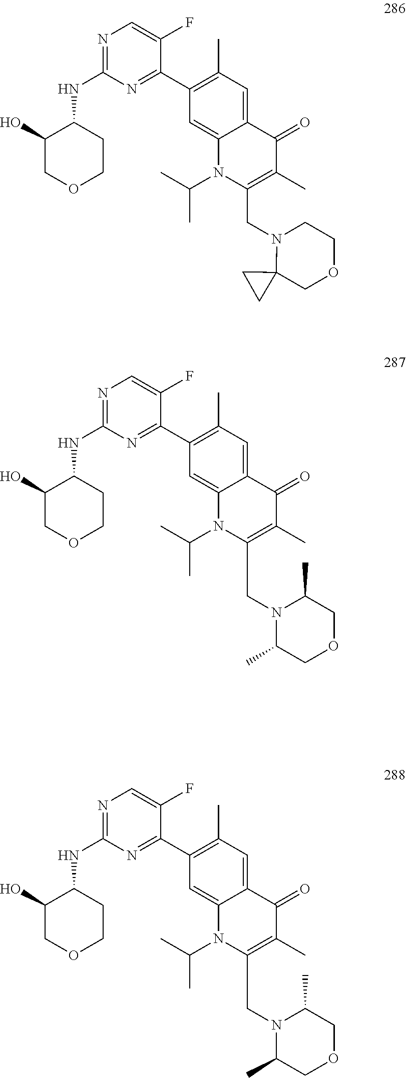 [SMILES: Cc1cc2c(=O)c(C)c(CN3CCOCC34CC4)n(C(C)C)c2cc1-c1nc(N[C@@H]2CCOC[C@H]2O)ncc1F ; Cc1cc2c(=O)c(C)c(CN3[C@@H](C)COC[C@@H]3C)n(C(C)C)c2cc1-c1nc(N[C@@H]2CCOC[C@H]2O)ncc1F ; Cc1cc2c(=O)c(C)c(CN3[C@H](C)COC[C@H]3C)n(C(C)C)c2cc1-c1nc(N[C@@H]2CCOC[C@H]2O)ncc1F]

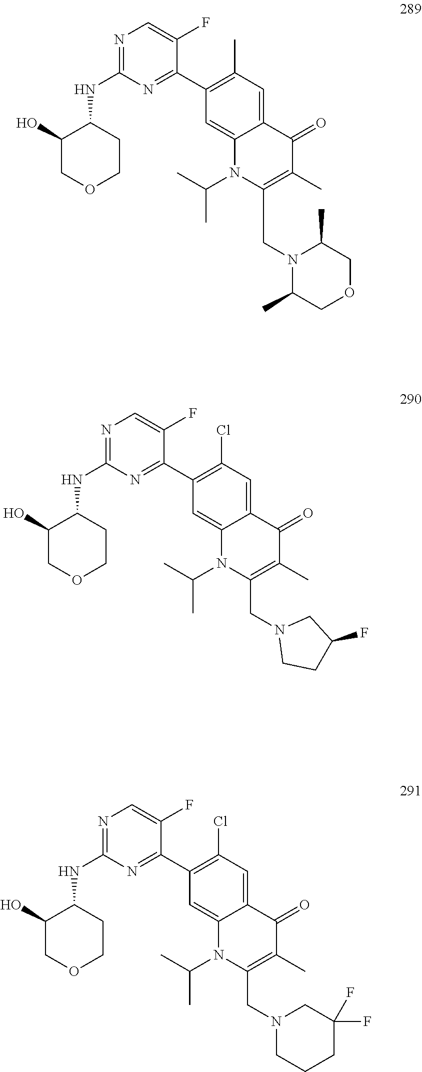 [SMILES: Cc1cc2c(=O)c(C)c(CN3[C@H](C)COC[C@@H]3C)n(C(C)C)c2cc1-c1nc(N[C@@H]2CCOC[C@H]2O)ncc1F ; Cc1c(CN2CC[C@H](F)C2)n(C(C)C)c2cc(-c3nc(N[C@@H]4CCOC[C@H]4O)ncc3F)c(Cl)cc2c1=O ; Cc1c(CN2CCCC(F)(F)C2)n(C(C)C)c2cc(-c3nc(N[C@@H]4CCOC[C@H]4O)ncc3F)c(Cl)cc2c1=O]

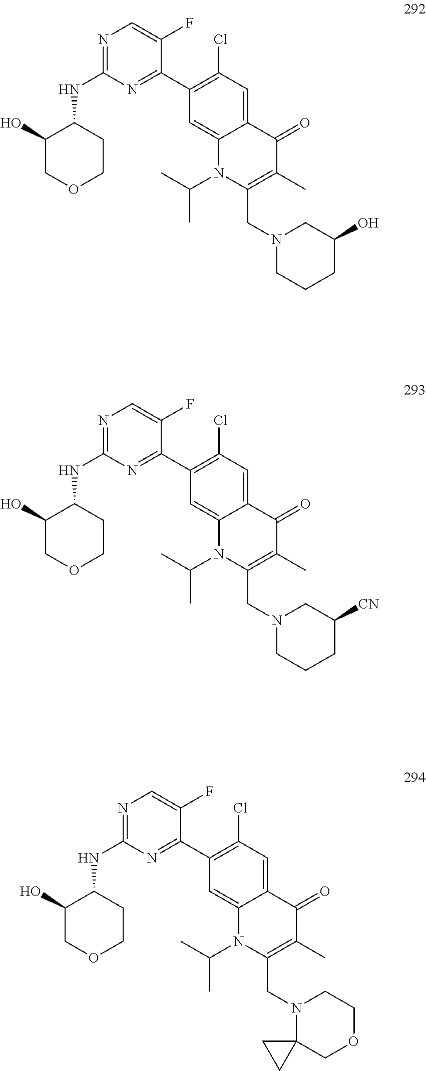 [SMILES: Cc1c(CN2CCC[C@H](O)C2)n(C(C)C)c2cc(-c3nc(N[C@@H]4CCOC[C@H]4O)ncc3F)c(Cl)cc2c1=O ; Cc1c(CN2CCC[C@H](C#N)C2)n(C(C)C)c2cc(-c3nc(N[C@@H]4CCOC[C@H]4O)ncc3F)c(Cl)cc2c1=O ; Cc1c(CN2CCOCC23CC3)n(C(C)C)c2cc(-c3nc(N[C@@H]4CCOC[C@H]4O)ncc3F)c(Cl)cc2c1=O]

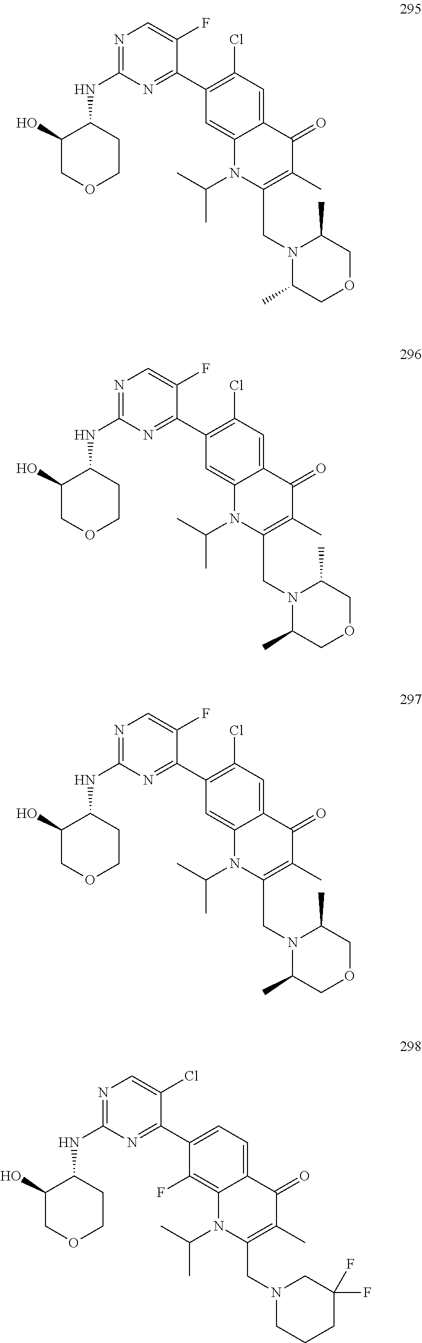 [SMILES: Cc1c(CN2[C@@H](C)COC[C@@H]2C)n(C(C)C)c2cc(-c3nc(N[C@@H]4CCOC[C@H]4O)ncc3F)c(Cl)cc2c1=O ; Cc1c(CN2[C@H](C)COC[C@H]2C)n(C(C)C)c2cc(-c3nc(N[C@@H]4CCOC[C@H]4O)ncc3F)c(Cl)cc2c1=O ; Cc1c(CN2[C@H](C)COC[C@@H]2C)n(C(C)C)c2cc(-c3nc(N[C@@H]4CCOC[C@H]4O)ncc3F)c(Cl)cc2c1=O ; Cc1c(CN2CCCC(F)(F)C2)n(C(C)C)c2c(F)c(-c3nc(N[C@@H]4CCOC[C@H]4O)ncc3Cl)ccc2c1=O]

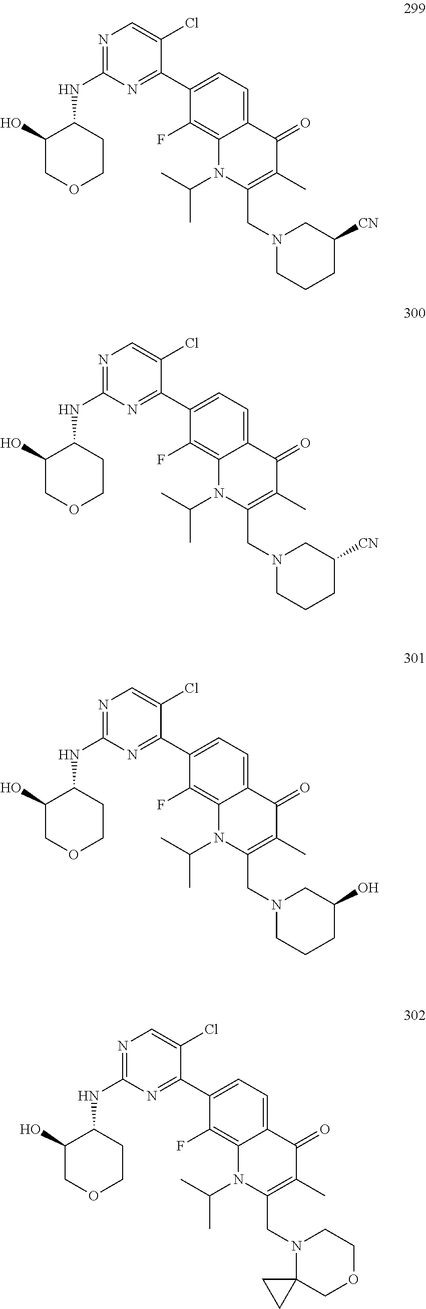 [SMILES: Cc1c(CN2CCC[C@H](C#N)C2)n(C(C)C)c2c(F)c(-c3nc(N[C@@H]4CCOC[C@H]4O)ncc3Cl)ccc2c1=O ; Cc1c(CN2CCC[C@@H](C#N)C2)n(C(C)C)c2c(F)c(-c3nc(N[C@@H]4CCOC[C@H]4O)ncc3Cl)ccc2c1=O ; Cc1c(CN2CCC[C@H](O)C2)n(C(C)C)c2c(F)c(-c3nc(N[C@@H]4CCOC[C@H]4O)ncc3Cl)ccc2c1=O ; Cc1c(CN2CCOCC23CC3)n(C(C)C)c2c(F)c(-c3nc(N[C@@H]4CCOC[C@H]4O)ncc3Cl)ccc2c1=O]

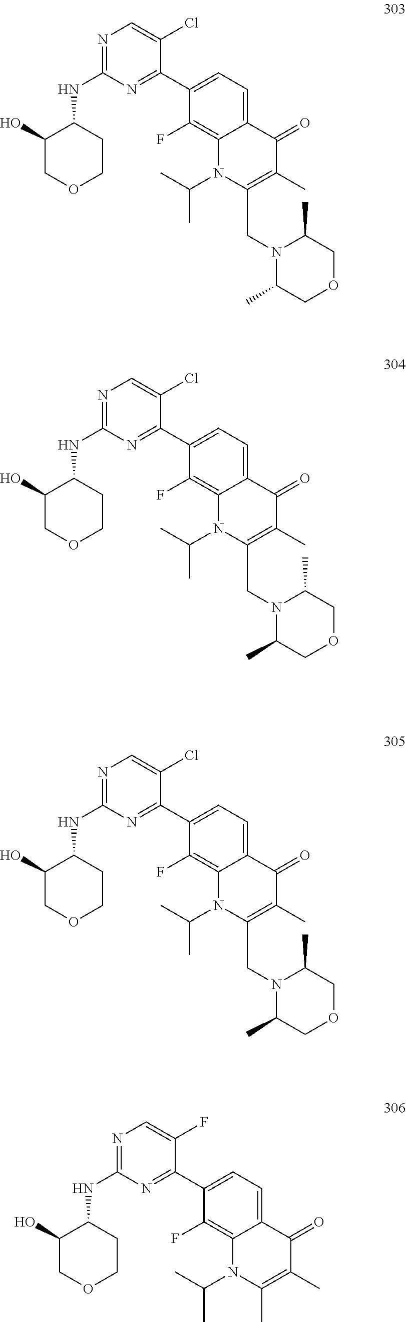 [SMILES: Cc1c(CN2[C@@H](C)COC[C@@H]2C)n(C(C)C)c2c(F)c(-c3nc(N[C@@H]4CCOC[C@H]4O)ncc3Cl)ccc2c1=O ; Cc1c(CN2[C@H](C)COC[C@H]2C)n(C(C)C)c2c(F)c(-c3nc(N[C@@H]4CCOC[C@H]4O)ncc3Cl)ccc2c1=O ; Cc1c(CN2[C@H](C)COC[C@@H]2C)n(C(C)C)c2c(F)c(-c3nc(N[C@@H]4CCOC[C@H]4O)ncc3Cl)ccc2c1=O ; Cc1c(C)n(C(C)C)c2c(F)c(-c3nc(N[C@@H]4CCOC[C@H]4O)ncc3F)ccc2c1=O]

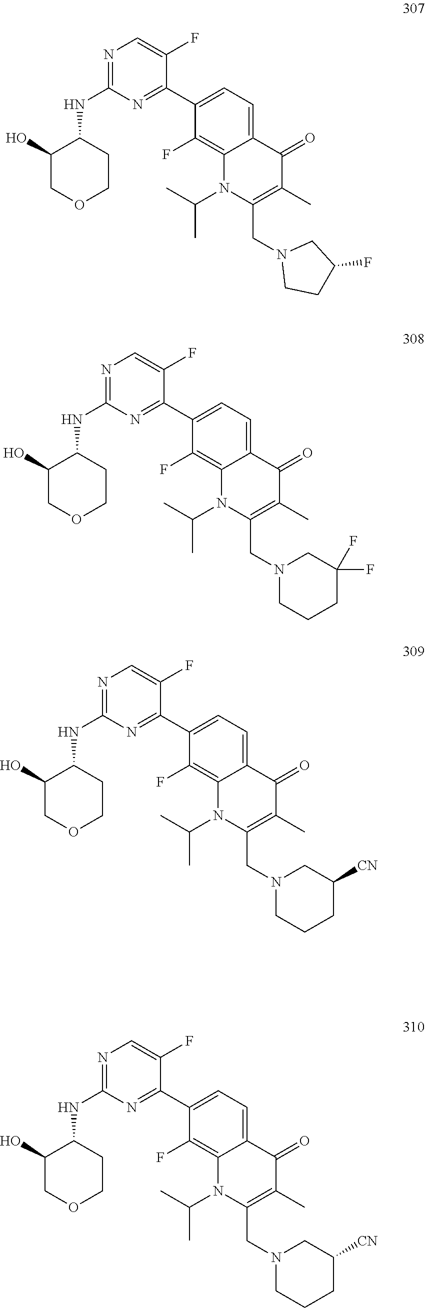 [SMILES: Cc1c(CN2CC[C@@H](F)C2)n(C(C)C)c2c(F)c(-c3nc(N[C@@H]4CCOC[C@H]4O)ncc3F)ccc2c1=O ; Cc1c(CN2CCCC(F)(F)C2)n(C(C)C)c2c(F)c(-c3nc(N[C@@H]4CCOC[C@H]4O)ncc3F)ccc2c1=O ; Cc1c(CN2CCC[C@H](C#N)C2)n(C(C)C)c2c(F)c(-c3nc(N[C@@H]4CCOC[C@H]4O)ncc3F)ccc2c1=O ; Cc1c(CN2CCC[C@@H](C#N)C2)n(C(C)C)c2c(F)c(-c3nc(N[C@@H]4CCOC[C@H]4O)ncc3F)ccc2c1=O]

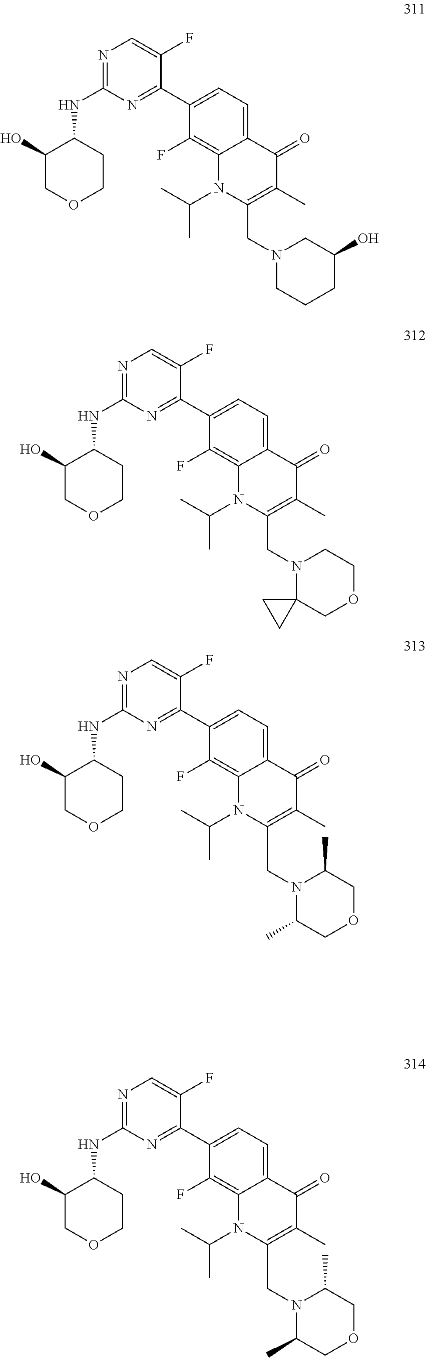 [SMILES: Cc1c(CN2CCC[C@H](O)C2)n(C(C)C)c2c(F)c(-c3nc(N[C@@H]4CCOC[C@H]4O)ncc3F)ccc2c1=O ; Cc1c(CN2CCOCC23CC3)n(C(C)C)c2c(F)c(-c3nc(N[C@@H]4CCOC[C@H]4O)ncc3F)ccc2c1=O ; Cc1c(CN2[C@@H](C)COC[C@@H]2C)n(C(C)C)c2c(F)c(-c3nc(N[C@@H]4CCOC[C@H]4O)ncc3F)ccc2c1=O ; Cc1c(CN2[C@H](C)COC[C@H]2C)n(C(C)C)c2c(F)c(-c3nc(N[C@@H]4CCOC[C@H]4O)ncc3F)ccc2c1=O]

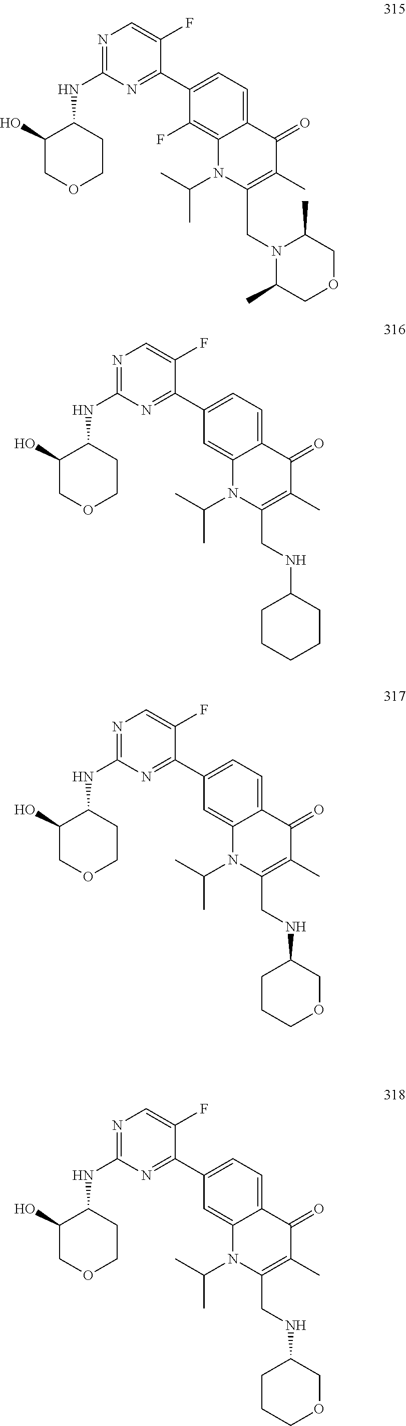 [SMILES: Cc1c(CN2[C@H](C)COC[C@@H]2C)n(C(C)C)c2c(F)c(-c3nc(N[C@@H]4CCOC[C@H]4O)ncc3F)ccc2c1=O ; Cc1c(CNC2CCCCC2)n(C(C)C)c2cc(-c3nc(N[C@@H]4CCOC[C@H]4O)ncc3F)ccc2c1=O ; Cc1c(CN[C@@H]2CCCOC2)n(C(C)C)c2cc(-c3nc(N[C@@H]4CCOC[C@H]4O)ncc3F)ccc2c1=O ; Cc1c(CN[C@H]2CCCOC2)n(C(C)C)c2cc(-c3nc(N[C@@H]4CCOC[C@H]4O)ncc3F)ccc2c1=O]

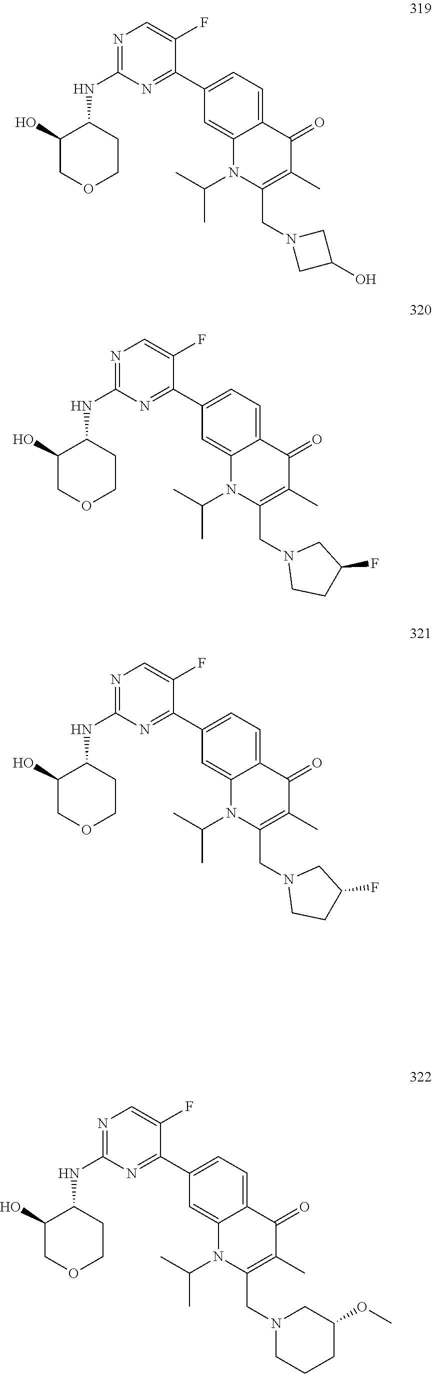 [SMILES: Cc1c(CN2CC(O)C2)n(C(C)C)c2cc(-c3nc(N[C@@H]4CCOC[C@H]4O)ncc3F)ccc2c1=O ; Cc1c(CN2CC[C@H](F)C2)n(C(C)C)c2cc(-c3nc(N[C@@H]4CCOC[C@H]4O)ncc3F)ccc2c1=O ; Cc1c(CN2CC[C@@H](F)C2)n(C(C)C)c2cc(-c3nc(N[C@@H]4CCOC[C@H]4O)ncc3F)ccc2c1=O ; CO[C@@H]1CCCN(Cc2c(C)c(=O)c3ccc(-c4nc(N[C@@H]5CCOC[C@H]5O)ncc4F)cc3n2C(C)C)C1]

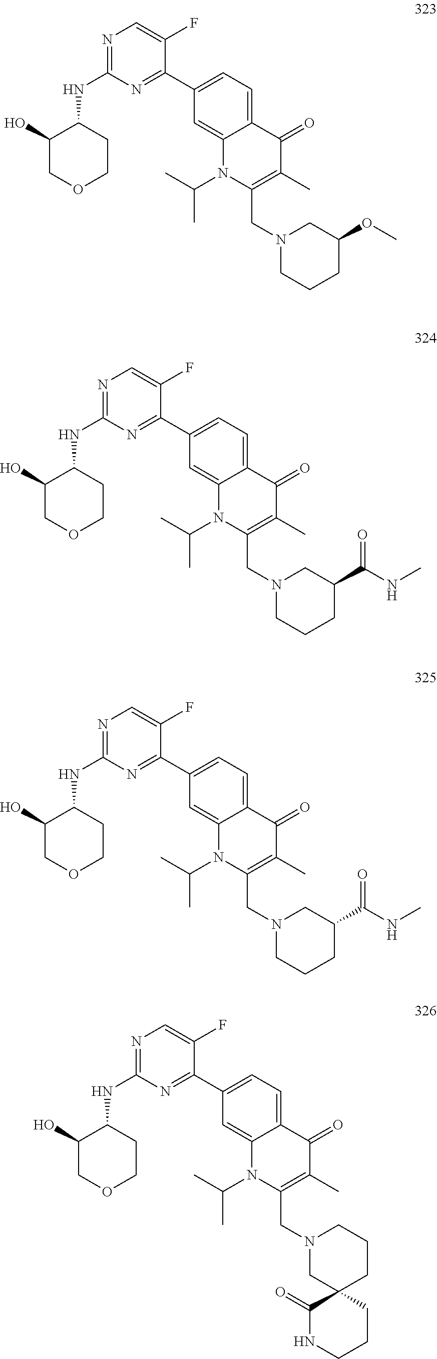 [SMILES: CO[C@H]1CCCN(Cc2c(C)c(=O)c3ccc(-c4nc(N[C@@H]5CCOC[C@H]5O)ncc4F)cc3n2C(C)C)C1 ; CNC(=O)[C@H]1CCCN(Cc2c(C)c(=O)c3ccc(-c4nc(N[C@@H]5CCOC[C@H]5O)ncc4F)cc3n2C(C)C)C1 ; CNC(=O)[C@@H]1CCCN(Cc2c(C)c(=O)c3ccc(-c4nc(N[C@@H]5CCOC[C@H]5O)ncc4F)cc3n2C(C)C)C1 ; Cc1c(CN2CCC[C@]3(CCCNC3=O)C2)n(C(C)C)c2cc(-c3nc(N[C@@H]4CCOC[C@H]4O)ncc3F)ccc2c1=O]

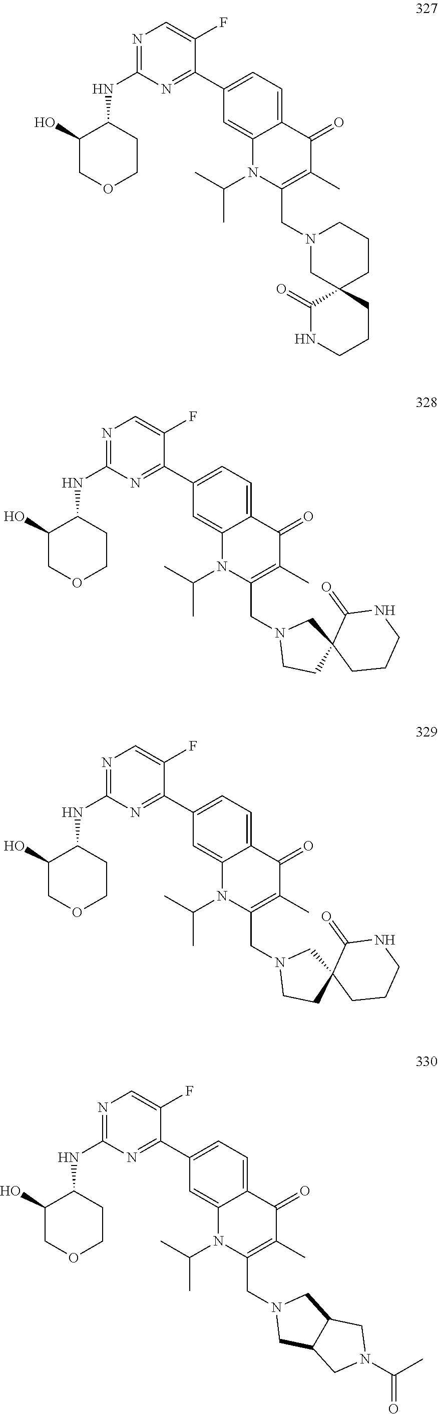

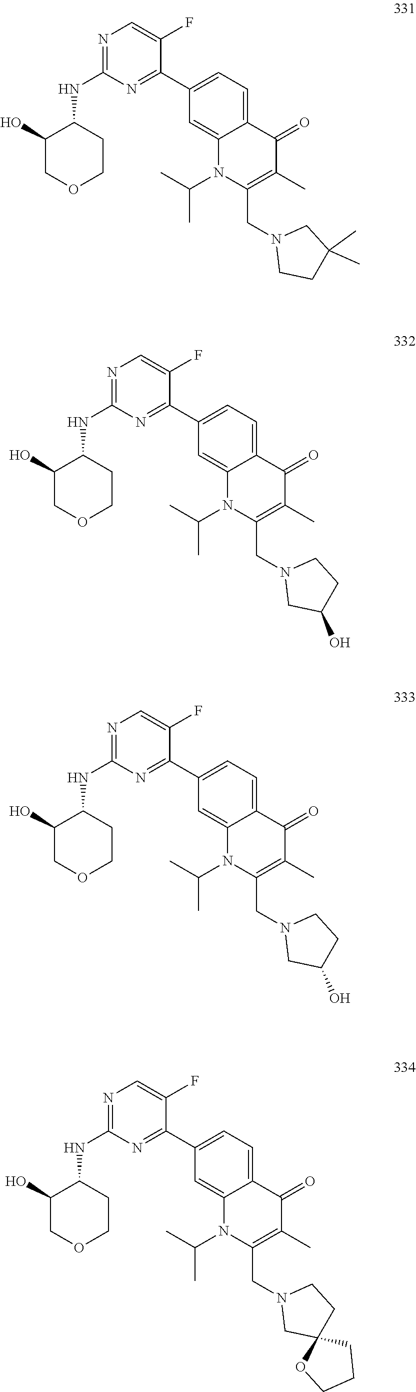 [SMILES: Cc1c(CN2CCC(C)(C)C2)n(C(C)C)c2cc(-c3nc(N[C@@H]4CCOC[C@H]4O)ncc3F)ccc2c1=O ; Cc1c(CN2CC[C@@H](O)C2)n(C(C)C)c2cc(-c3nc(N[C@@H]4CCOC[C@H]4O)ncc3F)ccc2c1=O ; Cc1c(CN2CC[C@H](O)C2)n(C(C)C)c2cc(-c3nc(N[C@@H]4CCOC[C@H]4O)ncc3F)ccc2c1=O ; Cc1c(CN2CC[C@]3(CCCO3)C2)n(C(C)C)c2cc(-c3nc(N[C@@H]4CCOC[C@H]4O)ncc3F)ccc2c1=O]

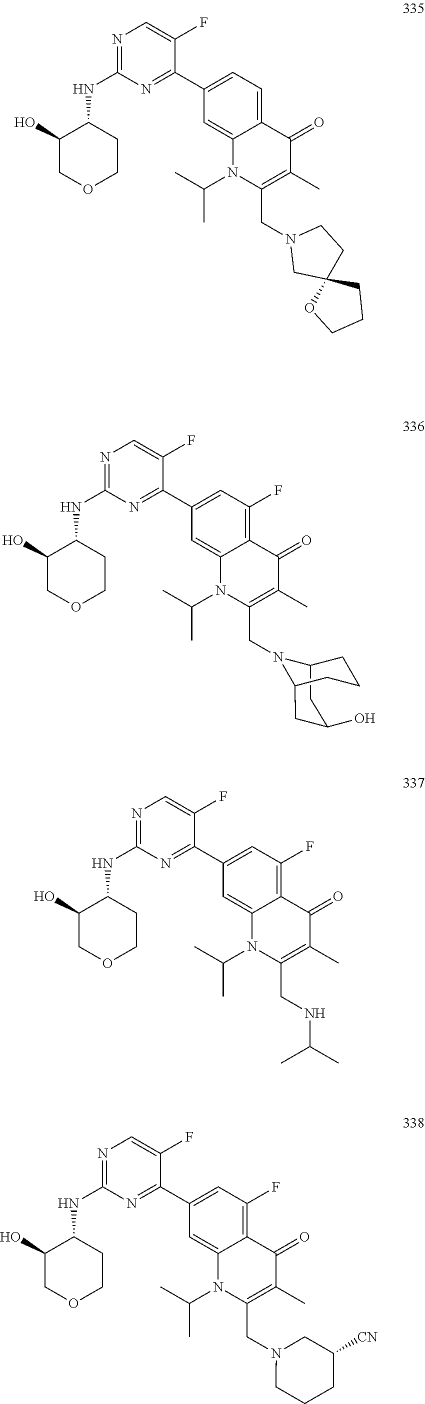 [SMILES: Cc1c(CN2CC[C@@]3(CCCO3)C2)n(C(C)C)c2cc(-c3nc(N[C@@H]4CCOC[C@H]4O)ncc3F)ccc2c1=O ; Cc1c(CN2C3CCCC2CC(O)C3)n(C(C)C)c2cc(-c3nc(N[C@@H]4CCOC[C@H]4O)ncc3F)cc(F)c2c1=O ; Cc1c(CNC(C)C)n(C(C)C)c2cc(-c3nc(N[C@@H]4CCOC[C@H]4O)ncc3F)cc(F)c2c1=O ; Cc1c(CN2CCC[C@@H](C#N)C2)n(C(C)C)c2cc(-c3nc(N[C@@H]4CCOC[C@H]4O)ncc3F)cc(F)c2c1=O]

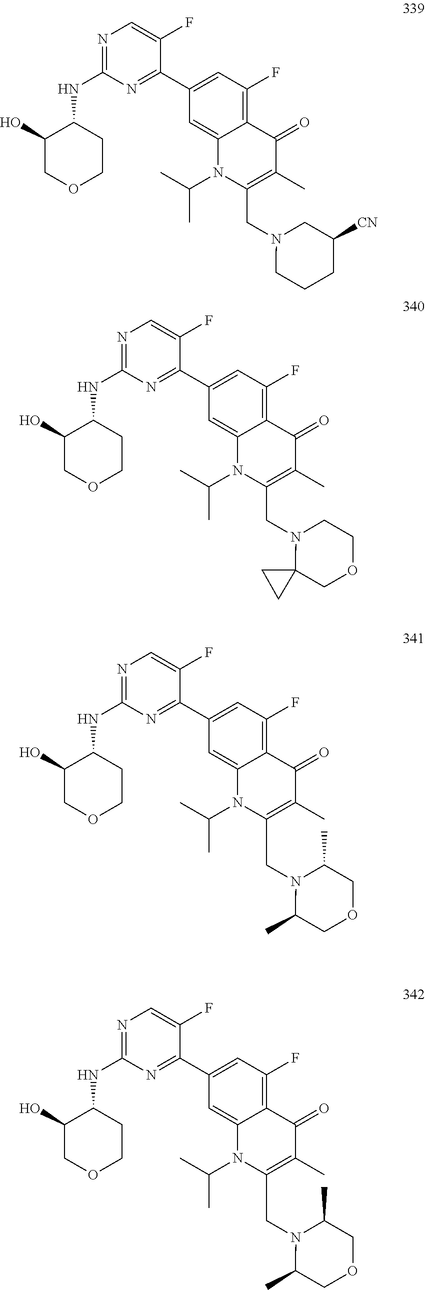 [SMILES: Cc1c(CN2CCC[C@H](C#N)C2)n(C(C)C)c2cc(-c3nc(N[C@@H]4CCOC[C@H]4O)ncc3F)cc(F)c2c1=O ; Cc1c(CN2CCOCC23CC3)n(C(C)C)c2cc(-c3nc(N[C@@H]4CCOC[C@H]4O)ncc3F)cc(F)c2c1=O ; Cc1c(CN2[C@H](C)COC[C@H]2C)n(C(C)C)c2cc(-c3nc(N[C@@H]4CCOC[C@H]4O)ncc3F)cc(F)c2c1=O ; Cc1c(CN2[C@H](C)COC[C@@H]2C)n(C(C)C)c2cc(-c3nc(N[C@@H]4CCOC[C@H]4O)ncc3F)cc(F)c2c1=O]

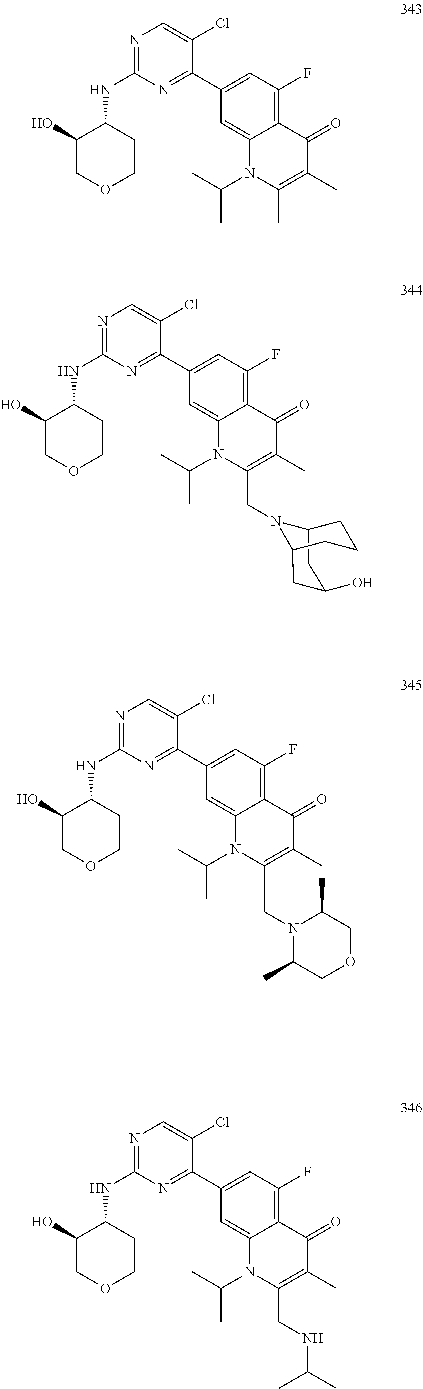 [SMILES: Cc1c(C)n(C(C)C)c2cc(-c3nc(N[C@@H]4CCOC[C@H]4O)ncc3Cl)cc(F)c2c1=O ; Cc1c(CN2C3CCCC2CC(O)C3)n(C(C)C)c2cc(-c3nc(N[C@@H]4CCOC[C@H]4O)ncc3Cl)cc(F)c2c1=O ; Cc1c(CN2[C@H](C)COC[C@@H]2C)n(C(C)C)c2cc(-c3nc(N[C@@H]4CCOC[C@H]4O)ncc3Cl)cc(F)c2c1=O ; Cc1c(CNC(C)C)n(C(C)C)c2cc(-c3nc(N[C@@H]4CCOC[C@H]4O)ncc3Cl)cc(F)c2c1=O]

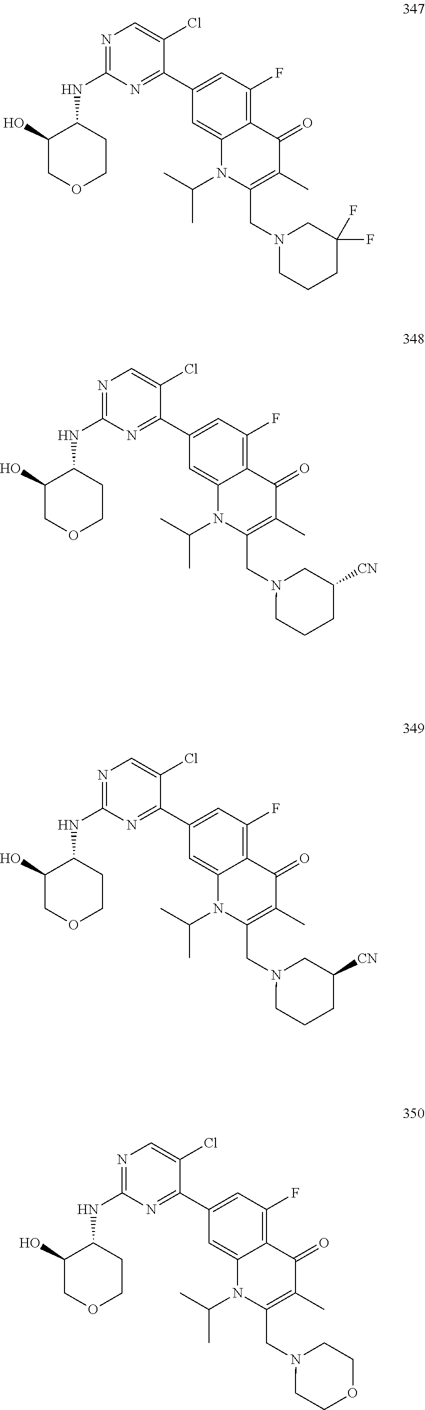 [SMILES: Cc1c(CN2CCCC(F)(F)C2)n(C(C)C)c2cc(-c3nc(N[C@@H]4CCOC[C@H]4O)ncc3Cl)cc(F)c2c1=O ; Cc1c(CN2CCC[C@@H](C#N)C2)n(C(C)C)c2cc(-c3nc(N[C@@H]4CCOC[C@H]4O)ncc3Cl)cc(F)c2c1=O ; Cc1c(CN2CCC[C@H](C#N)C2)n(C(C)C)c2cc(-c3nc(N[C@@H]4CCOC[C@H]4O)ncc3Cl)cc(F)c2c1=O ; Cc1c(CN2CCOCC2)n(C(C)C)c2cc(-c3nc(N[C@@H]4CCOC[C@H]4O)ncc3Cl)cc(F)c2c1=O]

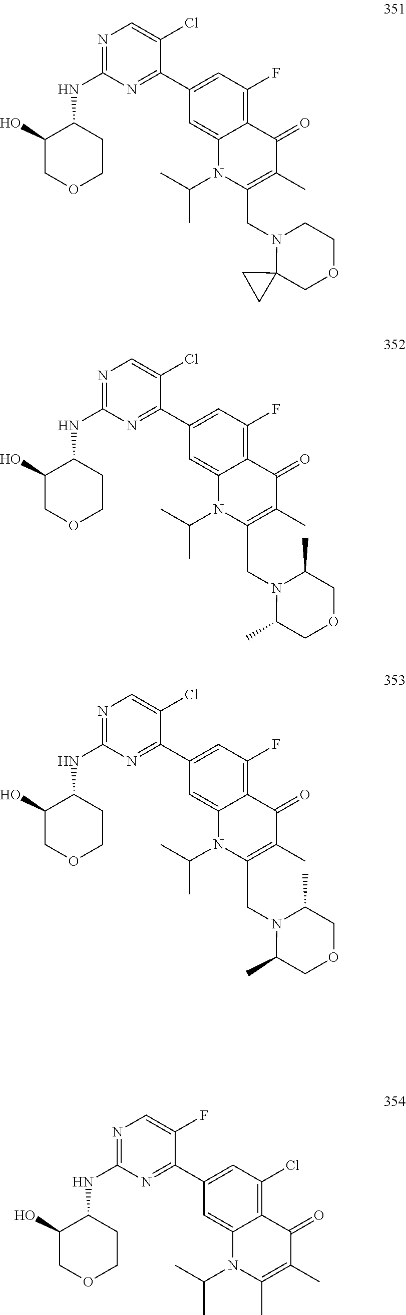 [SMILES: Cc1c(CN2CCOCC23CC3)n(C(C)C)c2cc(-c3nc(N[C@@H]4CCOC[C@H]4O)ncc3Cl)cc(F)c2c1=O ; Cc1c(CN2[C@@H](C)COC[C@@H]2C)n(C(C)C)c2cc(-c3nc(N[C@@H]4CCOC[C@H]4O)ncc3Cl)cc(F)c2c1=O ; Cc1c(CN2[C@H](C)COC[C@H]2C)n(C(C)C)c2cc(-c3nc(N[C@@H]4CCOC[C@H]4O)ncc3Cl)cc(F)c2c1=O ; Cc1c(C)n(C(C)C)c2cc(-c3nc(N[C@@H]4CCOC[C@H]4O)ncc3F)cc(Cl)c2c1=O]

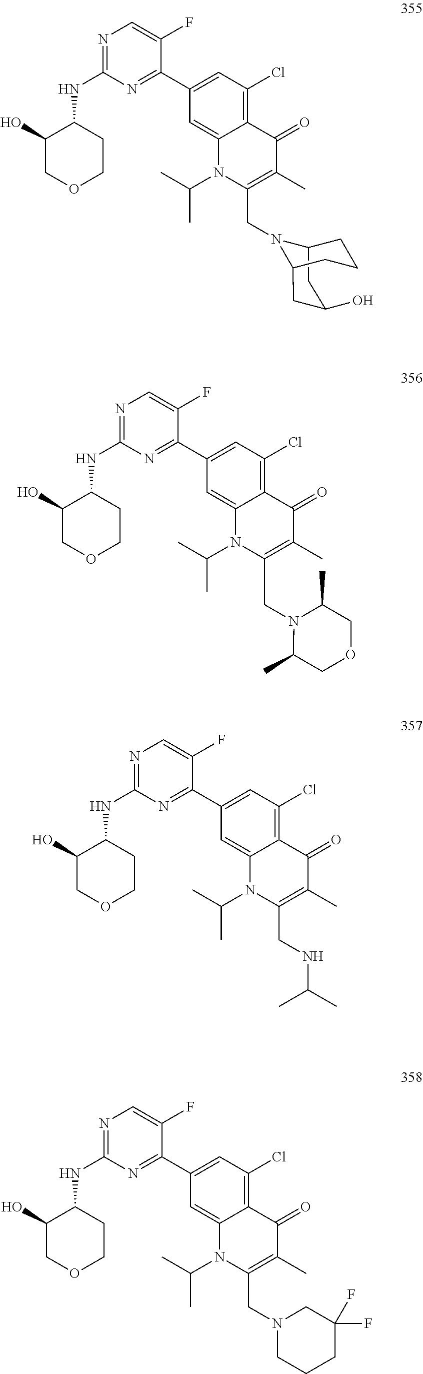 [SMILES: Cc1c(CN2C3CCCC2CC(O)C3)n(C(C)C)c2cc(-c3nc(N[C@@H]4CCOC[C@H]4O)ncc3F)cc(Cl)c2c1=O ; Cc1c(CN2[C@H](C)COC[C@@H]2C)n(C(C)C)c2cc(-c3nc(N[C@@H]4CCOC[C@H]4O)ncc3F)cc(Cl)c2c1=O ; Cc1c(CNC(C)C)n(C(C)C)c2cc(-c3nc(N[C@@H]4CCOC[C@H]4O)ncc3F)cc(Cl)c2c1=O ; Cc1c(CN2CCCC(F)(F)C2)n(C(C)C)c2cc(-c3nc(N[C@@H]4CCOC[C@H]4O)ncc3F)cc(Cl)c2c1=O]

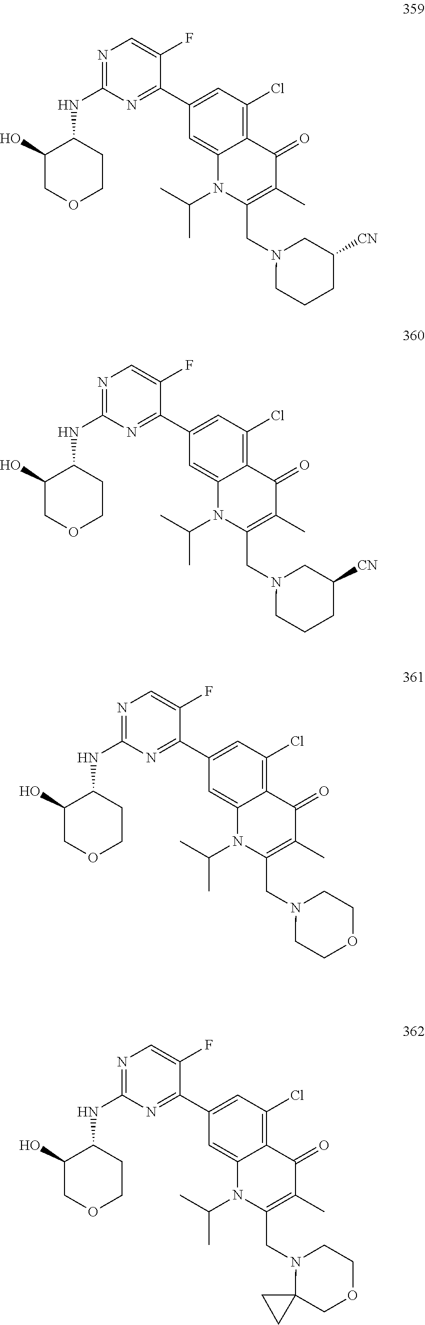 [SMILES: Cc1c(CN2CCC[C@@H](C#N)C2)n(C(C)C)c2cc(-c3nc(N[C@@H]4CCOC[C@H]4O)ncc3F)cc(Cl)c2c1=O ; Cc1c(CN2CCC[C@H](C#N)C2)n(C(C)C)c2cc(-c3nc(N[C@@H]4CCOC[C@H]4O)ncc3F)cc(Cl)c2c1=O ; Cc1c(CN2CCOCC2)n(C(C)C)c2cc(-c3nc(N[C@@H]4CCOC[C@H]4O)ncc3F)cc(Cl)c2c1=O ; Cc1c(CN2CCOCC23CC3)n(C(C)C)c2cc(-c3nc(N[C@@H]4CCOC[C@H]4O)ncc3F)cc(Cl)c2c1=O]

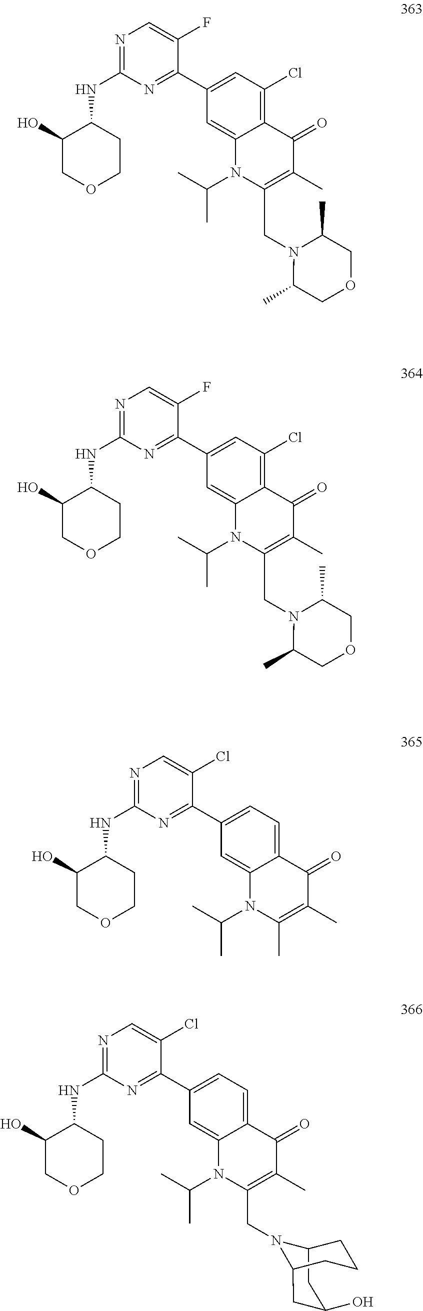 [SMILES: Cc1c(CN2[C@@H](C)COC[C@@H]2C)n(C(C)C)c2cc(-c3nc(N[C@@H]4CCOC[C@H]4O)ncc3F)cc(Cl)c2c1=O ; Cc1c(CN2[C@H](C)COC[C@H]2C)n(C(C)C)c2cc(-c3nc(N[C@@H]4CCOC[C@H]4O)ncc3F)cc(Cl)c2c1=O ; Cc1c(C)n(C(C)C)c2cc(-c3nc(N[C@@H]4CCOC[C@H]4O)ncc3Cl)ccc2c1=O ; Cc1c(CN2C3CCCC2CC(O)C3)n(C(C)C)c2cc(-c3nc(N[C@@H]4CCOC[C@H]4O)ncc3Cl)ccc2c1=O]

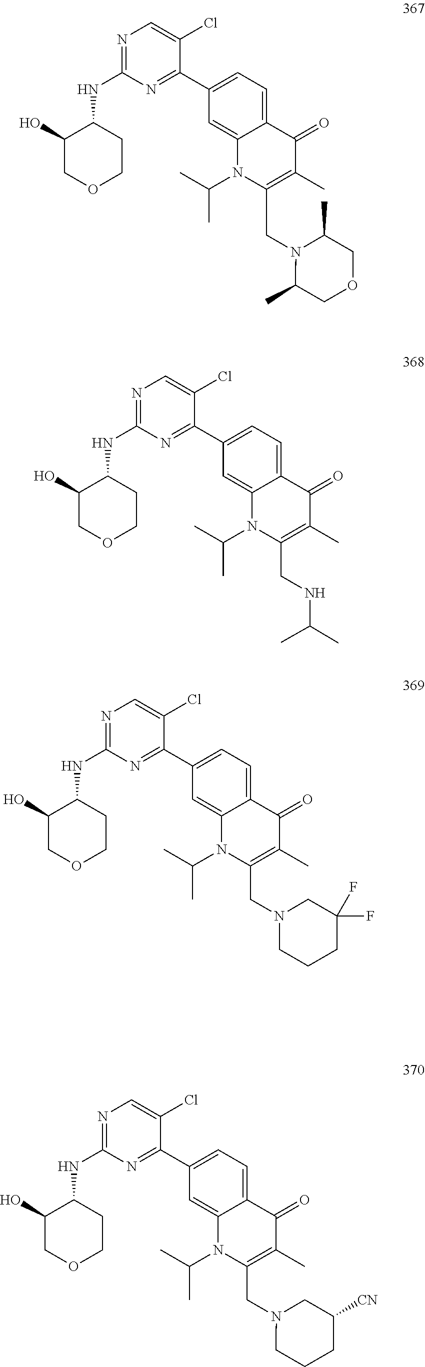 [SMILES: Cc1c(CN2[C@H](C)COC[C@@H]2C)n(C(C)C)c2cc(-c3nc(N[C@@H]4CCOC[C@H]4O)ncc3Cl)ccc2c1=O ; Cc1c(CNC(C)C)n(C(C)C)c2cc(-c3nc(N[C@@H]4CCOC[C@H]4O)ncc3Cl)ccc2c1=O ; Cc1c(CN2CCCC(F)(F)C2)n(C(C)C)c2cc(-c3nc(N[C@@H]4CCOC[C@H]4O)ncc3Cl)ccc2c1=O ; Cc1c(CN2CCC[C@@H](C#N)C2)n(C(C)C)c2cc(-c3nc(N[C@@H]4CCOC[C@H]4O)ncc3Cl)ccc2c1=O]

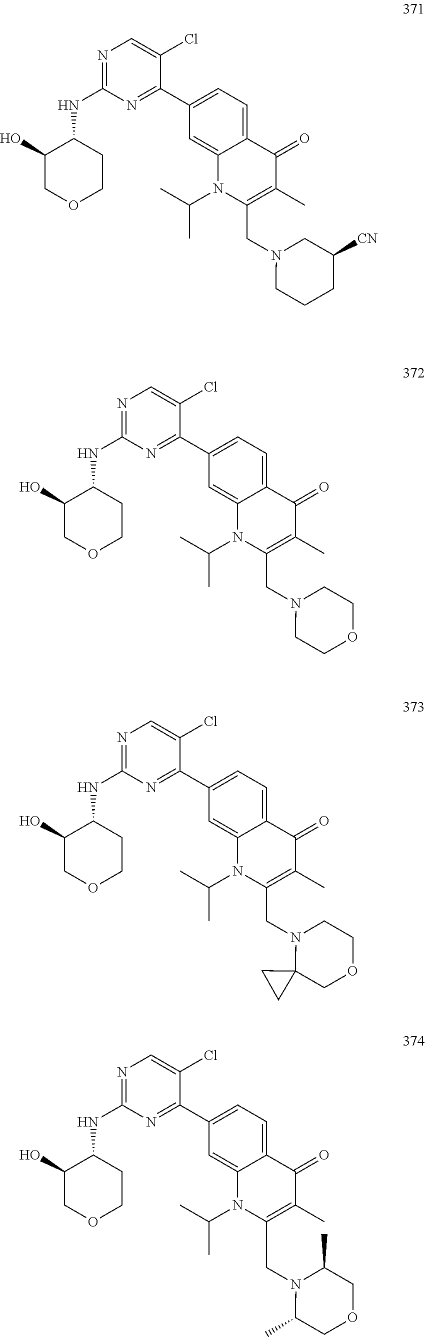 [SMILES: Cc1c(CN2CCC[C@H](C#N)C2)n(C(C)C)c2cc(-c3nc(N[C@@H]4CCOC[C@H]4O)ncc3Cl)ccc2c1=O ; Cc1c(CN2CCOCC2)n(C(C)C)c2cc(-c3nc(N[C@@H]4CCOC[C@H]4O)ncc3Cl)ccc2c1=O ; Cc1c(CN2CCOCC23CC3)n(C(C)C)c2cc(-c3nc(N[C@@H]4CCOC[C@H]4O)ncc3Cl)ccc2c1=O ; Cc1c(CN2[C@@H](C)COC[C@@H]2C)n(C(C)C)c2cc(-c3nc(N[C@@H]4CCOC[C@H]4O)ncc3Cl)ccc2c1=O]

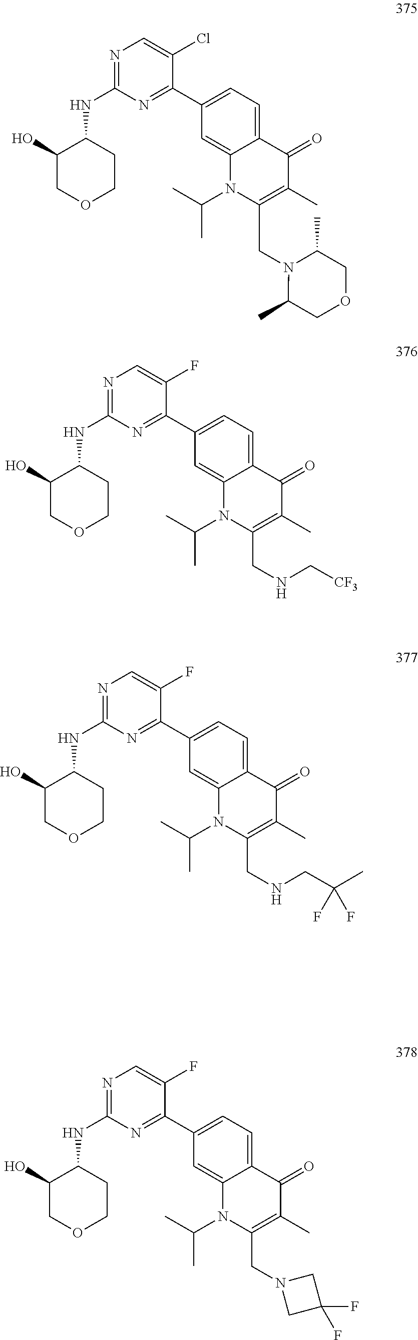 [SMILES: Cc1c(CN2[C@H](C)COC[C@H]2C)n(C(C)C)c2cc(-c3nc(N[C@@H]4CCOC[C@H]4O)ncc3Cl)ccc2c1=O ; Cc1c(CNCC(F)(F)F)n(C(C)C)c2cc(-c3nc(N[C@@H]4CCOC[C@H]4O)ncc3F)ccc2c1=O ; Cc1c(CNCC(C)(F)F)n(C(C)C)c2cc(-c3nc(N[C@@H]4CCOC[C@H]4O)ncc3F)ccc2c1=O ; Cc1c(CN2CC(F)(F)C2)n(C(C)C)c2cc(-c3nc(N[C@@H]4CCOC[C@H]4O)ncc3F)ccc2c1=O]

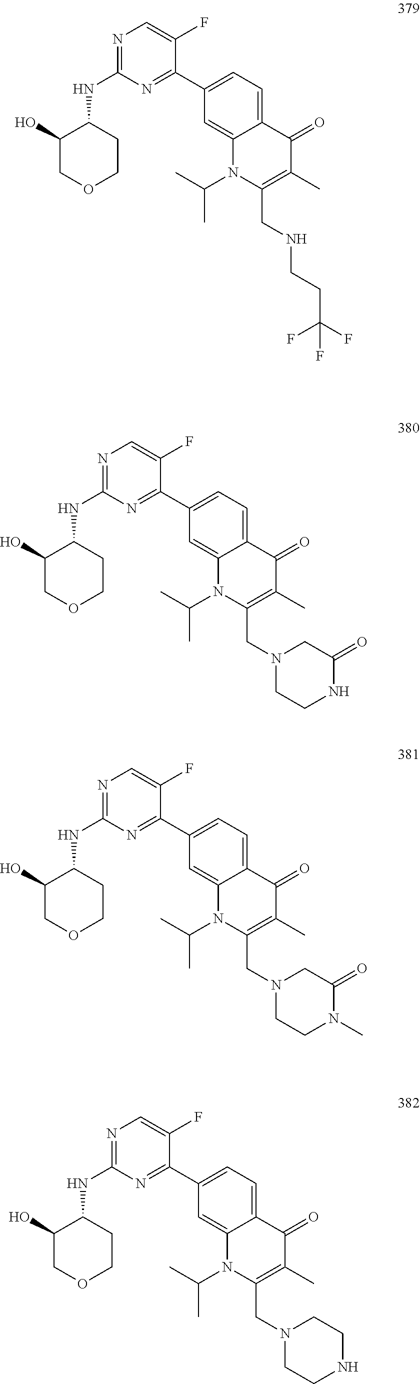 [SMILES: Cc1c(CNCCC(F)(F)F)n(C(C)C)c2cc(-c3nc(N[C@@H]4CCOC[C@H]4O)ncc3F)ccc2c1=O ; Cc1c(CN2CCNC(=O)C2)n(C(C)C)c2cc(-c3nc(N[C@@H]4CCOC[C@H]4O)ncc3F)ccc2c1=O ; Cc1c(CN2CCN(C)C(=O)C2)n(C(C)C)c2cc(-c3nc(N[C@@H]4CCOC[C@H]4O)ncc3F)ccc2c1=O ; Cc1c(CN2CCNCC2)n(C(C)C)c2cc(-c3nc(N[C@@H]4CCOC[C@H]4O)ncc3F)ccc2c1=O]

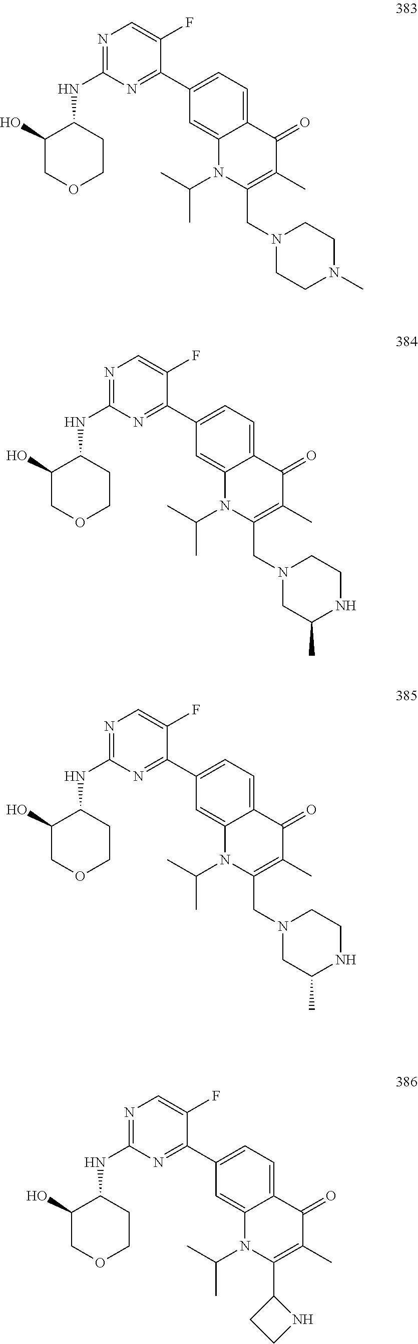 [SMILES: Cc1c(CN2CCN(C)CC2)n(C(C)C)c2cc(-c3nc(N[C@@H]4CCOC[C@H]4O)ncc3F)ccc2c1=O ; Cc1c(CN2CCN[C@@H](C)C2)n(C(C)C)c2cc(-c3nc(N[C@@H]4CCOC[C@H]4O)ncc3F)ccc2c1=O ; Cc1c(CN2CCN[C@H](C)C2)n(C(C)C)c2cc(-c3nc(N[C@@H]4CCOC[C@H]4O)ncc3F)ccc2c1=O ; Cc1c(C2CCN2)n(C(C)C)c2cc(-c3nc(N[C@@H]4CCOC[C@H]4O)ncc3F)ccc2c1=O]

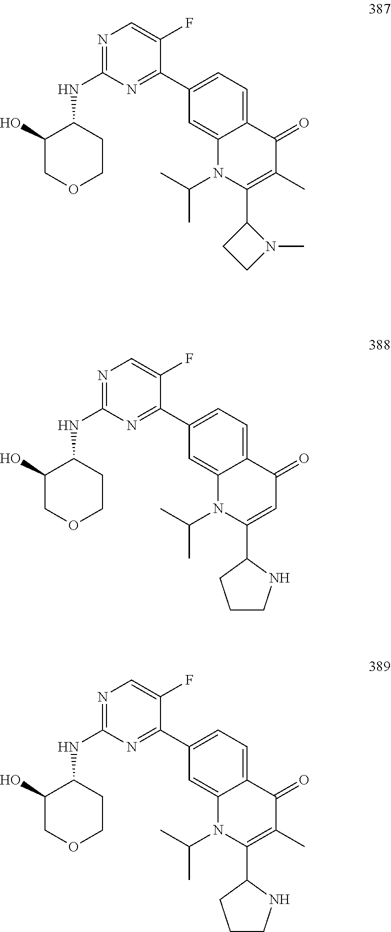 [SMILES: Cc1c(C2CCN2C)n(C(C)C)c2cc(-c3nc(N[C@@H]4CCOC[C@H]4O)ncc3F)ccc2c1=O ; CC(C)n1c(C2CCCN2)cc(=O)c2ccc(-c3nc(N[C@@H]4CCOC[C@H]4O)ncc3F)cc21 ; Cc1c(C2CCCN2)n(C(C)C)c2cc(-c3nc(N[C@@H]4CCOC[C@H]4O)ncc3F)ccc2c1=O]

20. A pharmaceutical composition comprising a compound of any one of claims 1-19 or a pharmaceutically acceptable salt, stereoisomer, tautomer or prodrug thereof, together with a pharmaceutically acceptable excipient.

21. A method of decreasing CDK4 activity by inhibition, which comprises administering to an individual the compound according to any one of claims 1-19, or a pharmaceutically acceptable salt thereof, including the compound of formula (I) or the specific compounds exemplified herein.

22. The method of claim 21, wherein the disease is selected from cancer, preferred breast cancer, lung cancer, pancreatic cancer, prostate cancer, bone cancer, liver cancer and endometrial cancer.

23. Use of a compound of any one of claims 1-19 or a pharmaceutically acceptable salt, stereoisomer, tautomer or prodrug thereof in the preparation of a medicament for treating a disease that can be affected by CDK4 modulation.

24. The use of claim 23, wherein the disease is cancer, preferred breast cancer, lung cancer, pancreatic cancer, prostate cancer, bone cancer, liver cancer and endometrial cancer.

25. A method of treating a disease or disorder in a patient comprising administering to the patient a therapeutically effective amount of the compound any one of claims 1-19, or a pharmaceutically acceptable salt thereof as a CKD4 kinase inhibitor, wherein the disease or disorder is associated with inhibition of CDK4.

26. The method of claim 25, wherein the disease is selected from cancer, preferred breast cancer, lung cancer, pancreatic cancer, prostate cancer, bone cancer, liver cancer and endometrial cancer.
